(19) United States
(12) Patent Application Publication
(10) Pub. No.: US 2024/0092779 A1
(43) Pub. Date: Mar. 21, 2024

(54) USP1 INHIBITORS AND USES THEREOF

(71) Applicant: Zentaur Therapeutics USA Inc., Waltham, MA (US)

(72) Inventors: Qibin SU, Acton, MA (US); Huawei CHEN, Carlisle, MA (US); Guosheng WU, Yardley, PA (US)

(21) Appl. No.: 18/343,982

(22) Filed: Jun. 29, 2023

Related U.S. Application Data
(60) Provisional application No. 63/367,268, filed on Jun. 29, 2022.

(51) Int. Cl. C07D 471/04; C07D 498/04; C07D 487/04; A61P 35/00

(52) U.S. Cl. CPC C07D 471/04; C07D 498/04; C07D 487/04; A61P 35/00

(57) ABSTRACT

Provided herein are inhibitors of ubiquitin-specific-processing Protease 1 (USP1), their pharmaceutical compositions, and methods of use for treating diseases or disorders, such as cancers described herein.

### CROSS REFERENCE TO RELATED APPLICATIONS

This application claims the benefit of U.S. Provisional Application 63/367,268, filed on Jun. 29, 2022, the entire contents of which is incorporated herein by reference in their entireties.

### TECHNICAL FIELD OF THE INVENTION

The present invention relates to compounds and methods useful for inhibition of a ubiquitin-specific-processing Protease 1 (USP1). The invention also provides pharmaceutically acceptable compositions comprising compounds of the present invention and methods of using said compositions in the treatment of various diseases, disorders, and conditions as described herein.

### BACKGROUND OF THE INVENTION

USP1 is a cysteine protease that belonging to the DUB family. It deubiquitinates a variety of cellular targets that play a role in DDR pathways, immune response, and cancer processes. USP1 interacts with UAF1 (USP1-associated factor 1) to form a stable complex that is required for the deubiquitinase activity. The well-characterized substrates of USP1/UAF1 complex, such as PCNA (proliferating cell nuclear antigen), and FANCD2 (Fanconi anemia group complementation group D2), are prominent players in the process of DNA translesion synthesis (TLS) and interstrand crosslink (ICL) repair, respectively. These two pathways are essential for repair of DNA damage included by DNA cross-linking agents, such as cisplatin and mitomycin C. Thus, USP1 is a critical regulator of genome integrity through the deubiqutination of Fanconi Anemia proteins and PCNA, and a potential synthetic lethal drug target for cancer cells that often have certain DDR pathway deficiencies.

### SUMMARY OF THE INVENTION

It has now been found that compounds of the present invention, and pharmaceutically acceptable compositions thereof, are effective as USP1 inhibitors. In one aspect, the present invention provides a compound of Formula I:

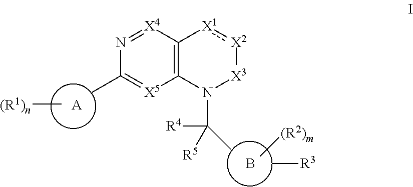

or a pharmaceutically acceptable salt thereof, wherein each variable is independently as defined herein and as described in embodiments herein.

Compounds of the present invention, and pharmaceutically acceptable salts and compositions thereof, are useful for treating a variety of diseases, disorders or conditions associated with USP1. Such diseases, disorders, or conditions include cellular proliferative disorders (e.g., cancer as described herein).

### BRIEF DESCRIPTION OF THE DRAWING

FIG. 1 depicts the tumor volume change after treatment.

FIG. 2 depicts the body weight change after treatment.

### DETAILED DESCRIPTION OF CERTAIN EMBODIMENTS

### 1. General Description of Certain Embodiments of the Invention:

Compounds of the present invention, and pharmaceutical salts and compositions thereof, are useful as inhibitors of USP1. Without wishing to be bound by any particular theory, it is believed that compounds of the present invention, and pharmaceutical compositions thereof, inhibit the activity of USP1, and thus treat diseases, disorders or conditions associated with USP1, such as cancer.

In one aspect, the present invention provides a compound of Formula I:

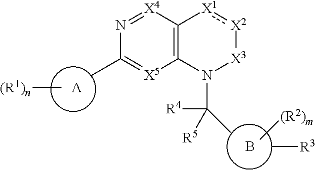

or a pharmaceutically acceptable salt thereof, wherein:

[symbol] is a single bond or a double bond;
X¹ is O, N, N(R¹¹), C(O), CR¹², or C(R¹²)₂;
X² is O, N, N(R¹¹), C(O), CR¹², or C(R¹²)₂;
X³ is N(R¹¹), C(O), or C(R¹²)₂;
X⁴ is N or CR¹²;
X⁵ is N or CR¹²;
each R¹¹ is independently hydrogen, deuterium, R, or —C(O)—R;
each R¹² is independently hydrogen, deuterium, halogen, R, —OR, —NHR, —N(R)₂, —C(O)—R, —COOR, —C(O)—NHR, or —C(O)—N(R)₂;
Ring A is a 5-6 membered heteroaromatic ring having 1-4 heteroatoms selected from N, O, and S;
each R¹ is independently halogen, R, —OR, —NHR, —N(R)₂, —C(O)—R, —C(O)—NHR, or —C(O)—N(R)₂;
Ring B is selected from a phenyl ring, a 5-6 membered heteroaromatic ring having 1-4 heteroatoms selected from N, O, and S, or a 5-6 membered heterocyclyl ring having 1-4 heteroatoms selected from N, O, and S;
each R² is independently halogen, R, —OR, —NHR, —N(R)₂, or —C(O)—R;
R³ is optionally substituted phenyl, or optionally substituted 5-6 membered heteroaryl having 1-4 heteroatoms selected from N, O, and S;
each of R⁴ and R⁵ is independently hydrogen, deuterium, halogen, R, —OR, —NHR, or —N(R) 2; or
R⁴ and R⁵, together with the atom to which they attach, form an optionally substituted ring selected from a C₃-C₇ carbocyclic ring or a 3-7 membered heterocyclic ring having 1-4 heteroatoms selected from N, O, and S;
each R is independently optionally substituted C₁₋₆ aliphatic, or an optionally substituted ring selected from a phenyl ring, a 5-6 membered heteroaromatic ring having 1-4 heteroatoms selected from N, O, and S, a C₃-C₇ carbocyclic ring, or a 3-7 membered heterocyclic ring having 1-4 heteroatoms selected from N, O, or S;
n is 0, 1, 2 or 3; and
m is 0, 1, 2 or 3.

### 2. Compounds and Definitions:

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

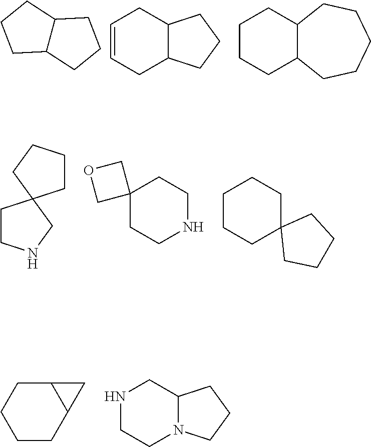

### Exemplary Bridged Bicyclics Include:

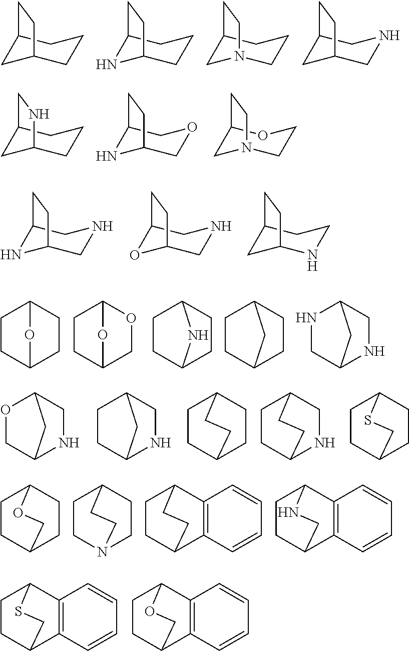

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺(as in N-substituted pyrrolidinyl)).

The term “unsaturated,” as used herein, means that a moiety has one or more units of unsaturation.

As used herein, the term “bivalent C_{1-[}(or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopromlenvl” refers to a bivalent cyclopropyl group of the following structure:

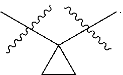 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{o}; —(CH₂)₀₋₄OR^{o}; —O(CH₂)₀₋₄R^{o}, —O—(CH₂)₀₋₄ C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN;—N₃; —(CH₂)₀₋₄N(R^{o})₂; —(CH₂)₀₋₄N(R^{o})C(O)R^{o}; —N(R^{o}C(S)R^{o}; —(CH₂)₀₋₄N(R^{o})C(O)NR^{o}₂; —N(R^{o})C(S)NR^{o}₂; —(CH₂)₀₋₄N(R^{o}C(O)OR^{o}; —N(R^{o})N(R^{o})C(O)R^{o}; —N(R^{o}N(R^{o}C(O)NR^{o}₂; —N(R^{o}N(R^{o}C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)O SiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o}R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o};—S(O)₂NR^{o}₂; —S(O)(NR^{o})R^{o}; —S(O)₂N═C(NR^{o}₂)₂; —(CH₂)₀₋₄S(O)R^{o}; —N(R^{o}S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched alkyl ene)C)(O)O—N(R^{o})₂.

Each R^{o} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{o} selected from ═O and ═S; or each R^{o} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂ CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃,—OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic,R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13 C— or 14 C-enriched carbon are within the scope of this invention. Such compounds are useful, for example, as analytical tools, as probes in biological assays, or as therapeutic agents in accordance with the present invention. In certain embodiments, a warhead moiety of a provided compound comprises one or more deuterium atoms.

As used herein, the terms “inhibitor” or “USP1 inhibitor” or “USP1 antagonist” are defined as a compound that binds to and/or inhibits USP1 with measurable affinity. In some embodiments, inhibition in the presence of the inhibitor is observed in a dose-dependent manner. In some embodiments, the measured signal (e.g., signaling activity or biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100% lower than the signal measured with a negative control under comparable conditions. The potency of an inhibitor is usually defined by its IC₅₀ value (half maximal inhibitory concentration or concentration required to inhibit 50% of the agonist response). The lower the IC₅₀ value the greater the potency of the antagonist and the lower the concentration that is required to inhibit the maximum biological response. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

The terms “measurable affinity” and “measurably inhibit,” as used herein, means a measurable change or inhibition in USP1 activity between a sample comprising a compound of the present invention, or composition thereof, and USP1, and an equivalent sample comprising USP1, in the absence of said compound, or composition thereof

### 3. Description of Exemplary Embodiments:

In one aspect, the present invention provides a compound of Formula I:

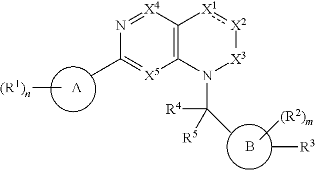

or a pharmaceutically acceptable salt thereof, wherein:
[symbol] is a single bond or a double bond;
X¹ is O, N, N(R¹¹), C(O), CR¹², or C(R¹²)₂;
X² is O, N, N(R¹¹), C(O), CR¹², or C(R¹²)₂;
X³ is N(R¹¹), C(O), or C(R¹²)₂;
X⁴ is N or CR¹²;
X⁵ is N or CR¹²;
each R¹¹ is independently hydrogen, deuterium, R, or —C(O)—R;
each R¹² is independently hydrogen, deuterium, halogen, R, —OR, —NHR, —N(R)₂, —C(O)—R, —COOR, —C(O)—NHR, or —C(O)—N(R)₂;
Ring A is a 5-6 membered heteroaromatic ring having 1-4 heteroatoms selected from N, O and S;
each R¹ is independently halogen, R, —OR, —NHR, —N(R)₂, —C(O)—R, —C(O)—NHR, or —C(O)—N(R)₂;
Ring B is selected from a phenyl ring, a 5-6 membered heteroaromatic ring having 1-4 heteroatoms selected from N, O, and S, or a 5-6 membered heterocyclyl ring having 1-4 heteroatoms selected from N, O, and S;
each R² is independently halogen, R, —OR, —NHR, —N(R)₂, or —C(O)—R;
R³ is optionally substituted phenyl, or optionally substituted 5-6 membered heteroaryl having 1-4 heteroatoms selected from N, O, and S;
each of R⁴ and R⁵ is independently hydrogen, deuterium, halogen, R, —OR, —NHR, or —N(R) 2; or
R⁴ and R⁵, together with the atom to which they attach, form an optionally substituted ring selected from a C₃-C₇ carbocyclic ring or a 3-7 membered heterocyclic ring having 1-4 heteroatoms selected from N, O, and S;
each R is independently optionally substituted C₁₋₆ aliphatic, or an optionally substituted ring selected from a phenyl ring, a 5-6 membered heteroaromatic ring having 1-4 heteroatoms selected from N, O, and S, a C₃-C₇ carbocyclic ring, or a 3-7 membered heterocyclic ring having 1-4 heteroatoms selected from N, O, or S;
n is 0, 1, 2 or 3; and
m is 0, 1, 2 or 3.

As defined generally above, [symbol] is a single bond or a double bond.

In some embodiments, [symbol] is a single bond. In some embodiments, [symbol] is a double bond.

In some embodiments, [symbol] is selected from those depicted in Table 1, below.

As defined generally above, X¹ is O, N, N(R¹¹), C(O), CR¹², or C(R¹²)₂, wherein each variable is independently as defined herein and as described in embodiments herein.

In some embodiments, X¹ is 0. In some embodiments, X¹ is N. In some embodiments, X¹ is N(R¹¹), wherein R¹¹ is as defined herein and as described in embodiments herein. In some embodiments, X¹ is C(O). In some embodiments, X¹ is CR¹², wherein R¹² is as defined herein and as described in embodiments herein. In some embodiments, X¹ is C(R¹²)₂, wherein each R¹² is as defined herein and as described in embodiments herein.

In some embodiments, X¹ is selected from those depicted in Table 1, below.

As defined generally above, X² is O, N, N(R¹¹), C(O), CR¹², or C(R¹²)₂, wherein each variable is independently as defined herein and as described in embodiments herein.

In some embodiments, X² is O. In some embodiments, X² is N. In some embodiments, X² is N(R¹¹), wherein R¹¹ is as defined herein and as described in embodiments herein. In some embodiments, X² is C(O). In some embodiments, X² is CR¹², wherein R¹² is as defined herein and as described in embodiments herein. In some embodiments, X² is C(R¹²)₂, wherein each R¹² is as defined herein and as described in embodiments herein.

In some embodiments, X² is selected from those depicted in Table 1, below.

As defined generally above, X³ is N(R¹¹), C(O), or C(R¹²)₂, wherein each variable is independently as defined herein and as described in embodiments herein.

In some embodiments, X³ is N(R¹¹), wherein R¹¹ is as defined herein and as described in embodiments herein. In some embodiments, X³ is C(O). In some embodiments, X³ is C(R¹²)₂, wherein each R¹² is as defined herein and as described in embodiments herein.

In some embodiments, X³ is selected from those depicted in Table 1, below.

As defined generally above, X⁴ is N or CR¹², wherein R¹² is as defined herein and as described in embodiments herein.

In some embodiments, X⁴ is N. In some embodiments, X³ is CR¹², wherein R¹² is as defined herein and as described in embodiments herein.

In some embodiments, X⁴ is selected from those depicted in Table 1, below.

As defined generally above, X⁵ is N or CR¹², wherein R¹² is as defined herein and as described in embodiments herein.

In some embodiments, X⁵ is N. In some embodiments, X⁵ is CR¹², wherein R¹² is as defined herein and as described in embodiments herein.

In some embodiments, X⁵ is selected from those depicted in Table 1, below.

As defined generally above, each R¹¹ is independently hydrogen, deuterium, R, or —C(O)—R, wherein each R is independently as defined herein and as described in embodiments herein.

In some embodiments, R¹¹ is hydrogen. In some embodiments, R¹¹ is deuterium. In some embodiments, R¹¹ is R, wherein each R is independently as defined herein and as described in embodiments herein. In some embodiments, R¹¹ is —C(O)—R, wherein each R is independently as defined herein and as described in embodiments herein.

In some embodiments, R¹¹ is —CH₃ or —CD₃.

In some embodiments, R¹¹ is selected from those depicted in Table 1, below.

As defined generally above, each R¹² is independently hydrogen, deuterium, halogen, R, —OR, —NHR, —N(R)₂, —C(O)—R, —COOR, —C(O)—NHR, or —C(O)—N(R)₂, wherein each R is independently as defined herein and as described in embodiments herein.

In some embodiments, R¹² is hydrogen. In some embodiments, R¹² is deuterium. In some embodiments, R¹² is halogen. In some embodiments, R¹² is R, wherein R is as defined herein and as described in embodiments herein. In some embodiments, R¹² is —OR, wherein R is as defined herein and as described in embodiments herein. In some embodiments, R¹² is —NHR, wherein R is as defined herein and as described in embodiments herein. In some embodiments, R¹² is —N(R)₂, wherein each R is independently as defined herein and as described in embodiments herein. In some embodiments, R¹² is —C(O)—R, wherein R is as defined herein and as described in embodiments herein. In some embodiments, R¹² is —COOR, wherein R is as defined herein and as described in embodiments herein. In some embodiments, R¹² is —C(O)—NHR, wherein R is as defined herein and as described in embodiments herein. In some embodiments, R¹² is —C(O)—N(R)₂, wherein each R is independently as defined herein and as described in embodiments herein.

In some embodiments, R¹² is optionally substituted

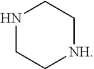

In some embodiments, R¹² is optionally substituted

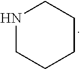

In some embodiments, R¹² is optionally substituted

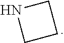

In some embodiments, R¹² is optionally substituted

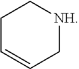

In some embodiments, R¹² is optionally substituted

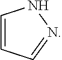

In some embodiments, R¹² is optionally substituted

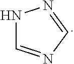

In some embodiments, R¹² is optionally substituted

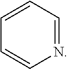

In some embodiments, each R¹² is independently H, —F, —Br, —Cl, —CF₃, —CHF₂, —CH₃, —CD₃,

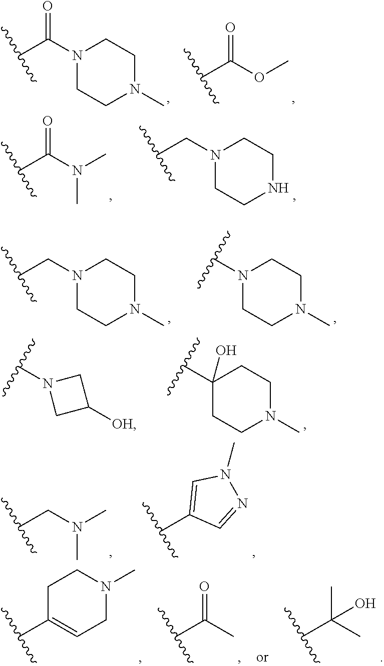

In some embodiments, R¹² is

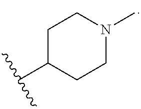 [SMILES: *C1CCN(C)CC1]

In some embodiments, R¹² is

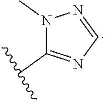 [SMILES: *c1ncnn1C]

In some embodiments, R¹² is

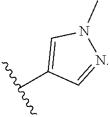 [SMILES: *c1cnn(C)c1]

In some embodiments, R¹² is

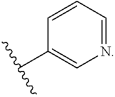 [SMILES: *c1cccnc1]

In some embodiments, R¹² is

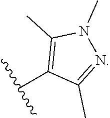 [SMILES: *c1c(C)nn(C)c1C]

In some embodiments, R¹² is

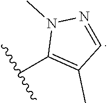 [SMILES: *c1c(C)cnn1C]

In some embodiments, R¹² is

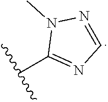 [SMILES: *c1ncnn1C]

In some embodiments, R¹² is

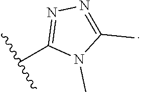 [SMILES: *c1nnc(C)n1C]

In some embodiments, R¹² is selected from those depicted in Table 1, below.

As defined generally above, Ring A is a 5-6 membered heteroaromatic ring having 1-4 heteroatoms selected from N, O, and S.

In some embodiments, Ring A is a 5 membered heteroaromatic ring having 1, 2, 3, or 4 heteroatoms selected from N, O, and S. In some embodiments, Ring A is a 6 membered heteroaromatic ring having 1, 2, 3, or 4 heteroatoms selected from N, O, and S.

In some embodiments, Ring A is a 5 or 6 membered heteroaromatic ring having 1, 2, 3, or 4 heteroatom N.

In some embodiments, Ring A is

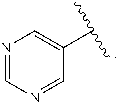 [SMILES: *c1cncnc1]

In some embodiments, Ring A is

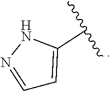 [SMILES: *c1ccn[nH]1]

In some embodiments,

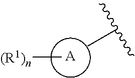

is

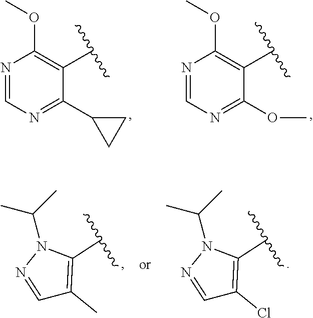

In some embodiments, Ring A is selected from those depicted in Table 1, below.

As defined generally above, each R¹ is independently halogen, R, —OR, —NHR, —N(R)₂, —C(O)—R, —C(O)—NHR, or —C(O)—N(R)₂, wherein each R is independently as defined herein and as described in embodiments herein.

In some embodiments, R¹ is halogen. In some embodiments, R¹ is R, wherein R is as defined herein and as described in embodiments herein. In some embodiments, R¹ is —OR, wherein R is as defined herein and as described in embodiments herein. In some embodiments, le is —NHR, wherein R is as defined herein and as described in embodiments herein. In some embodiments, R¹ is —N(R)₂, wherein each R is independently as defined herein and as described in embodiments herein. In some embodiments, R¹ is —C(O)—R, wherein R is as defined herein and as described in embodiments herein. In some embodiments, R¹ is —C(O)—NHR, wherein R is as defined herein and as described in embodiments herein. In some embodiments, R¹ is —C(O)—N(R)₂, wherein each R is independently as defined herein and as described in embodiments herein.

In some embodiments, each R¹ is independently —Cl, —F, —OCH₃, —OCD₃,

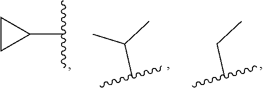

—O—CHF₂, —O—CF₃, or

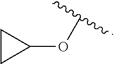 [SMILES: *OC1CC1]

In some embodiments, R¹ is selected from those depicted in Table 1, below.

As defined generally above, Ring B is selected from a phenyl ring, a 5-6 membered heteroaromatic ring having 1-4 heteroatoms selected from N, O, and S, or a 5-6 membered heterocyclyl ring having 1-4 heteroatoms selected from N, O, and S.

In some embodiments, Ring B is a phenyl ring.

In some embodiments, Ring B is a 5-6 membered heteroaromatic ring having 1, 2, 3, or 4 heteroatoms selected from N, O, and S. In some embodiments, Ring B is a 5 membered heteroaromatic ring having 1, 2, 3, or 4 heteroatoms selected from N, O, and S. In some embodiments, Ring B is a 6 membered heteroaromatic ring having 1, 2, 3, or 4 heteroatoms selected from N, O, and S. In some embodiments, Ring A is a 5 or 6 membered heteroaromatic ring having 1, 2, 3, or 4 heteroatom N.

In some embodiments, Ring B is a 5 or 6 membered heterocyclyl ring having 1, 2, 3, or 4 heteroatoms selected from N, O, and S. In some embodiments, Ring B is a 5 or 6 membered heterocyclyl ring having 1, 2, 3, or 4 heteroatom N.

In some embodiments, Ring B is

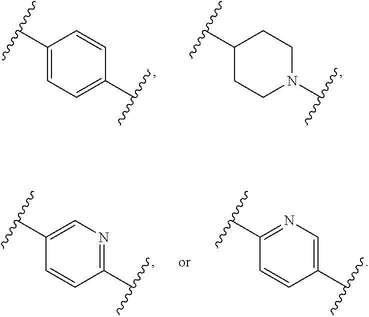 [SMILES: *c1ccc(*)cc1 ; *C1CCN(*)CC1 ; *c1ccc(*)nc1 ; *c1ccc(*)nc1]

In some embodiments, Ring B is selected from those depicted in Table 1, below.

As defined generally above, each R² is independently halogen, R, —OR, —NHR, —N(R)₂, or —C(O)—R, wherein each R is independently as defined herein and as described in embodiments herein.

In some embodiments, R² is halogen. In some embodiments, R² is R, wherein R is as defined herein and as described in embodiments herein. In some embodiments, R² is —OR, wherein R is as defined herein and as described in embodiments herein. In some embodiments, R² is —NHR, wherein R is as defined herein and as described in embodiments herein. In some embodiments, R² is —N(R)₂, wherein each R is independently as defined herein and as described in embodiments herein. In some embodiments, R² is —C(O)—R, wherein R is as defined herein and as described in embodiments herein.

In some embodiments, each R² is independently —F, —OCH₃, or —OCD₃.

In some embodiments, R² is selected from those depicted in Table 1, below.

In some embodiments,

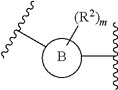

is

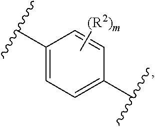

which is selected from

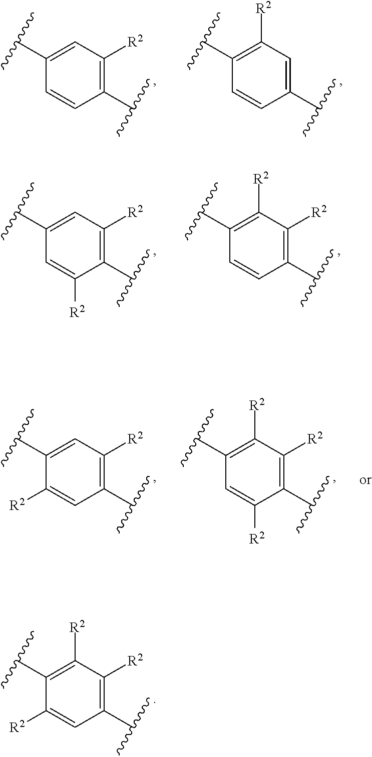

In some embodiments,

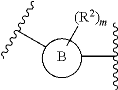

is

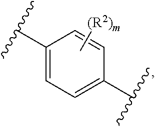

which is selected from

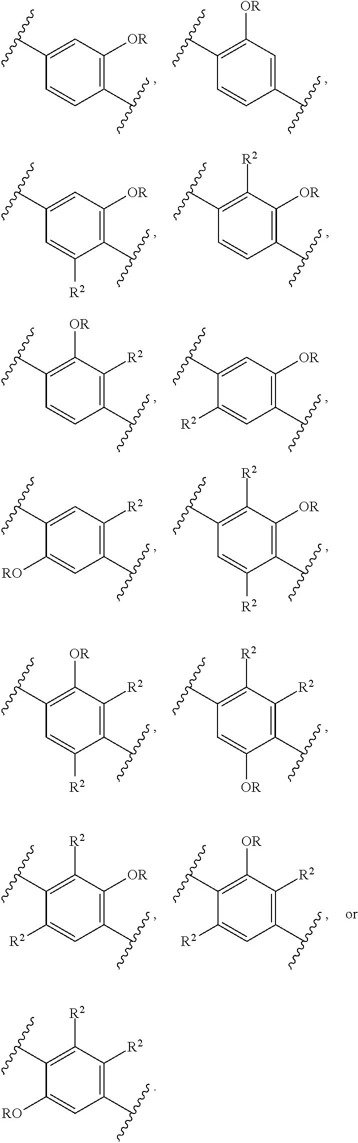

In some embodiments,

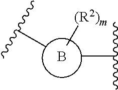

is selected from

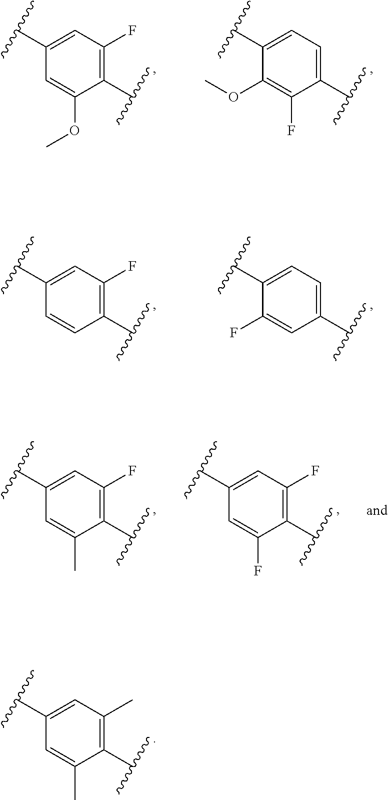

In some embodiments,

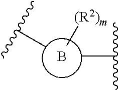

is

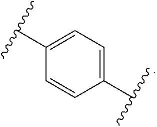 [SMILES: *c1ccc(*)cc1]

In some embodiments,

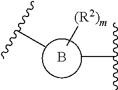

is

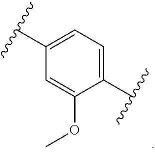 [SMILES: *c1ccc(*)c(OC)c1]

As defined generally above, R³ is optionally substituted phenyl, or optionally substituted 5-6 membered heteroaryl having 1-4 heteroatoms selected from N, O, and S.

In some embodiments, R³ is optionally substituted phenyl.

In some embodiments, R³ is optionally substituted 5 or 6 membered heteroaryl having 1, 2, 3, or 4 heteroatoms selected from N, O, and S. In some embodiments, R³ is optionally substituted 5 membered heteroaryl having 1, 2, 3, or 4 heteroatoms selected from N, O, and S. In some embodiments, R³ is optionally substituted 6 membered heteroaryl having 1, 2, 3, or 4 heteroatoms selected from N, O, and S. In some embodiments, R³ is optionally substituted 5 or 6 membered heteroaryl having 1, 2, 3, or 4 heteroatom N.

In some embodiments, R³ is optionally substituted

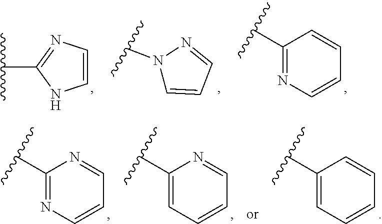

In some embodiments, R³ is

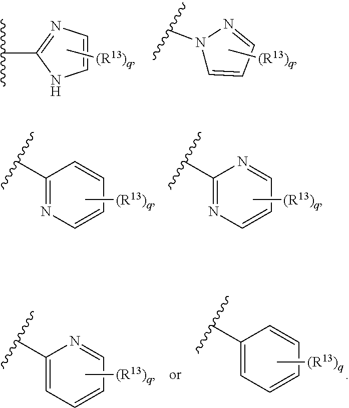

wherein each R¹³ is independently halogen, R, or —OR, and q is 0, 1, 2, or 3.

In some embodiments, R¹³ is halogen. In some embodiments, R¹³ is R, wherein R is as defined herein and as described in embodiments herein. In some embodiments, R¹³ is —OR, wherein R is as defined herein and as described in embodiments herein.

In some embodiments, R¹³ is —F, —CH₃, —CD₃, —CF₃, —CHF₂, —OCH₃, —OCD₃,

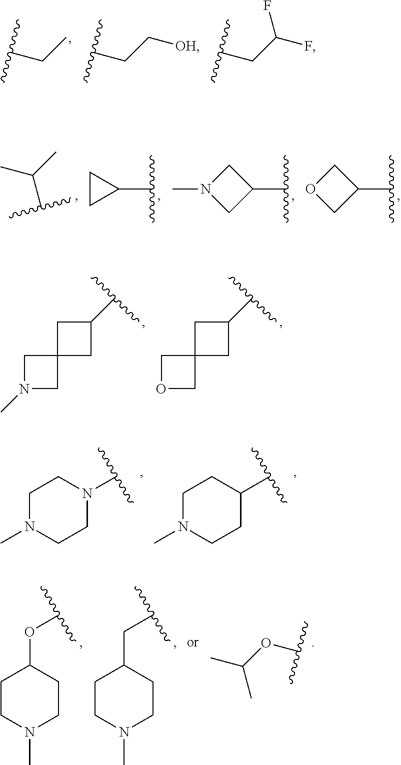

In some embodiments, q is 0. In some embodiments, q is 1. In some embodiments, q is 2. In some embodiments, q is 3.

In some embodiments, R³ is

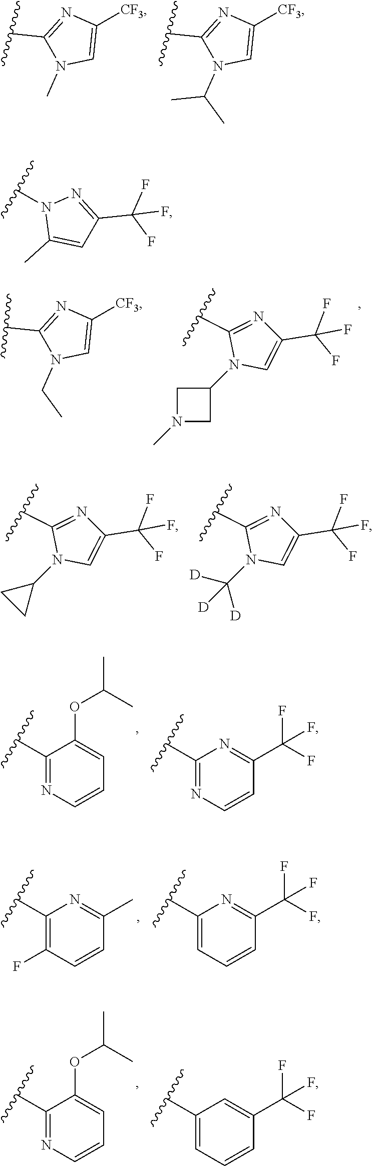

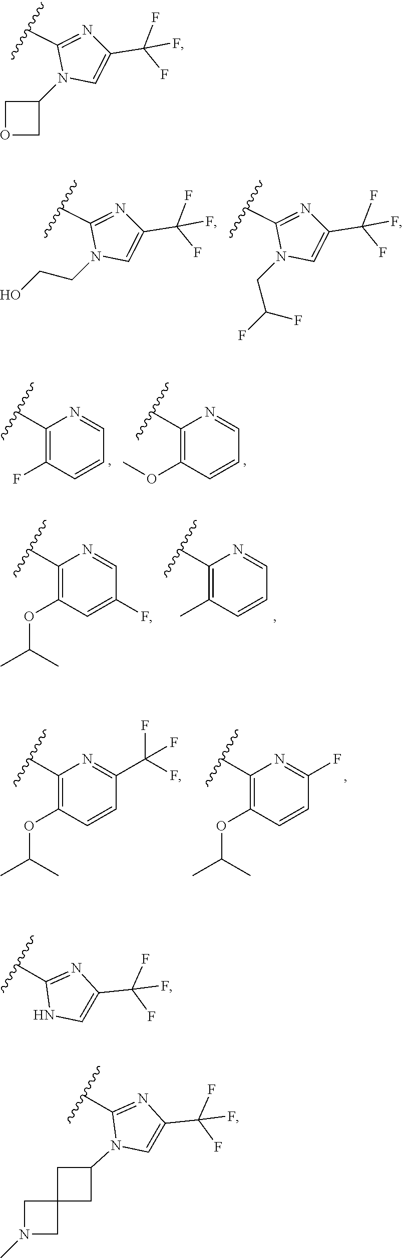

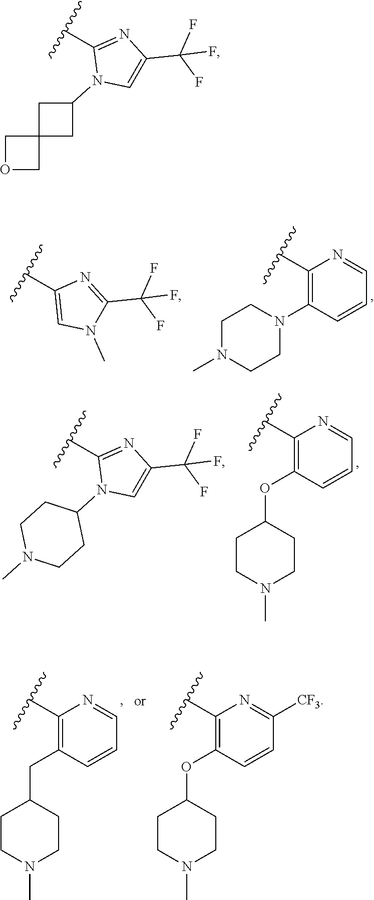

In some embodiments, R³ is

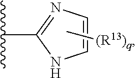

which is

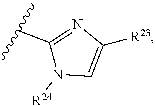

wherein R²³ is optionally substituted C₁₋₆ aliphatic, and R²⁴ is optionally substituted C₃-C₇ carbocyclic ring. In some embodiments, R²³ is —CH₃, —C₃, —CF₃, —CHF₂,

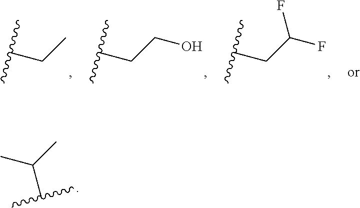

In some embodiments, R²⁴ is

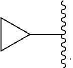 [SMILES: *C1CC1]

In some embodiments, R³ is selected from those depicted in Table 1, below.

As defined generally above, each of R⁴ and R⁵ is independently hydrogen, deuterium, halogen, R, —OR, —NHR, or —N(R)₂; or R⁴ and R⁵, together with the atom to which they attach, form an optionally substituted ring selected from a C₃-C₇ carbocyclic ring or a 3-7 membered heterocyclic ring having 1-4 heteroatoms selected from N, O, and S, wherein each R is independently as defined herein and as described in embodiments herein.

In some embodiments, R⁴ and R⁵ is independently hydrogen, deuterium, halogen, R, —OR, —NHR, or —N(R)₂, wherein each R is independently as defined herein and as described in embodiments herein.

In some embodiments, R⁴ is hydrogen. In some embodiments, R⁴ is deuterium. In some embodiments, R⁴ is halogen. In some embodiments, R⁴ is R, wherein R is as defined herein and as described in embodiments herein. In some embodiments, R⁴ is —OR, wherein R is as defined herein and as described in embodiments herein. In some embodiments, R⁴ is —NHR, wherein R is as defined herein and as described in embodiments herein. In some embodiments, R⁴ is —N(R)₂, wherein each R is independently as defined herein and as described in embodiments herein.

In some embodiments, R⁵ is hydrogen. In some embodiments, R⁵ is deuterium. In some embodiments, R⁵ is halogen. In some embodiments, R⁵ is R, wherein R is as defined herein and as described in embodiments herein. In some embodiments, R⁵ is —OR, wherein R is as defined herein and as described in embodiments herein. In some embodiments, R⁵ is —NHR, wherein R is as defined herein and as described in embodiments herein. In some embodiments, R⁵ is —N(R)₂, wherein each R is independently as defined herein and as described in embodiments herein.

In some embodiments, R⁴ and R⁵, together with the atom to which they attach, form an optionally substituted ring selected from a C₃-C₇ carbocyclic ring or a 3-7 membered heterocyclic ring having 1-4 heteroatoms selected from N, O, and S. In some embodiments, R⁴ and R⁵, together with the atom to which they attach, form an optionally substituted C₃-C₇ carbocyclic ring. In some embodiments, R⁴ and R⁵, together with the atom to which they attach, form an optionally substituted 3, 4, 5, 6, or 7 membered heterocyclic ring having 1, 2, 3, or 4 heteroatoms selected from N, O, and S. In some embodiments, R⁴ and R⁵, together with the atom to which they attach, form an optionally substituted 3, 4, 5, 6, or 7 membered heterocyclic ring having 1, 2, 3, or 4 heteroatoms selected from N and O. In some embodiments, R⁴ and R⁵, together with the atom to which they attach, form an optionally substituted 4, 5, or 6 membered heterocyclic ring having 1, 2, or 3 heteroatoms selected from N, O, and S. In some embodiments, R⁴ and R⁵, together with the atom to which they attach, form an optionally substituted 3, 4, 5, 6, or 7 membered heterocyclic ring having 1, 2, 3, or 4 heteroatom N. In some embodiments, R⁴ and R⁵, together with the atom to which they attach, form an optionally substituted 4, 5, or 6 membered heterocyclic ring having 1, 2, or 3 heteroatom N.

In some embodiments, each of R⁴ and R⁵ is independently selected from those depicted in Table 1, below.

As defined generally above, each R is independently optionally substituted C₁₋₆ aliphatic, or an optionally substituted ring selected from a phenyl ring, a 5-6 membered heteroaromatic ring having 1-4 heteroatoms selected from N, O, and S, a C₃-C₇ carbocyclic ring, or a 3-7 membered heterocyclic ring having 1-4 heteroatoms selected from N, O, or S.

In some embodiments, R is optionally substituted C₁₋₆ aliphatic. In some embodiments, R is unsubstituted C₁₋₆ aliphatic. In some embodiments, R is C₁₋₆ aliphatic substituted 1, 2, 3, 4, 5, or 6 times by -halogen. In some embodiments, R is C₁₋₆ aliphatic substituted 1, 2, 3, 4, 5, or 6 times by —F. In some embodiments, R is —C₁₋₃ aliphatic substituted 1, 2, 3, 4, 5, or 6 times by —F. In some embodiments, R is —CH₃. In some embodiments, R is —CH₂CH₃. In some embodiments, R is —CF₃. In some embodiments, R is —CHF₂.

In some embodiments, R is an optionally substituted phenyl ring.

In some embodiments, R is an optionally substituted 5 or 6 membered heteroaromatic ring having 1, 2, 3, or 4 heteroatoms selected from N, O, and S. In some embodiments, R is an optionally substituted 5 membered heteroaromatic ring having 1, 2, 3, or 4 heteroatoms selected from N, O, and S. In some embodiments, R is an optionally substituted 6 membered heteroaromatic ring having 1, 2, 3, or 4 heteroatoms selected from N, O, and S. In some embodiments, R is an optionally substituted 5 or 6 membered heteroaromatic ring having 1, 2, 3, or 4 heteroatom N.

In some embodiments, R is an optionally substituted C₃-C₇ carbocyclic ring.

In some embodiments, R is an optionally substituted 3, 4, 5, 6, or 7 membered heterocyclic ring having 1, 2, 3, or 4 heteroatoms selected from N, O, and S. In some embodiments, R is an optionally substituted 3, 4, 5, 6, or 7 membered heterocyclic ring having 1, 2, 3, or 4 heteroatoms selected from N and O. In some embodiments, R is an optionally substituted 4, 5, or 6 membered heterocyclic ring having 1, 2, or 3 heteroatoms selected from N, O, and S. In some embodiments, R is an optionally substituted 3, 4, 5, 6, or 7 membered heterocyclic ring having 1, 2, 3, or 4 heteroatom N. In some embodiments, R is an optionally substituted 4, 5, or 6 membered heterocyclic ring having 1, 2, or 3 heteroatom N.

In some embodiments, R is unsubstituted C₁₋₆ aliphatic. In some embodiments, R is unsubstituted C₁₋₆ alkyl. In some embodiments, R is C₁₋₆ aliphatic substituted 1, 2, 3, 4, 5, or 6 times by halogen or —OH. In some embodiments, R is C₁₋₆ alkyl substituted 1, 2, 3, 4, 5, or 6 times by halogen or —OH. In some embodiments, R is —CH₃, —CD₃,

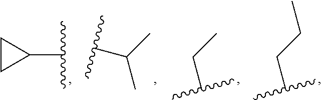

—CH₂F, —CHF₂, or —CF₃.

In some embodiments, R is optionally substituted

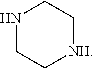

In some embodiments, R is

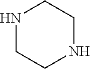

optionally substituted 1, 2, or 3 times by halogen or C₁₋₆ alkyl. In some embodiments, R is optionally substituted

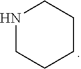

In some embodiments, R is

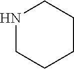

optionally substituted 1, 2, or 3 times by halogen or C₁₋₆ alkyl. In some embodiments, R is optionally substituted

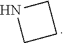

In some embodiments, R is

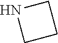

optionally substituted 1, 2, or 3 times by halogen or C₁₋₆ alkyl. In some embodiments, R is optionally substituted

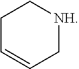

In some embodiments, R is

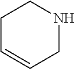

optionally substituted 1, 2, or 3 times by halogen or C₁₋₆ alkyl.

In some embodiments, R is optionally substituted

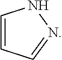

In some embodiments, R is

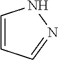

optionally substituted 1, 2, or 3 times by halogen or C₁₋₆ alkyl. In some embodiments, R is optionally substituted

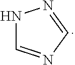

In some embodiments, R is

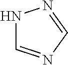

optionally substituted 1, 2, or 3 times by halogen or C₁₋₆ alkyl. In some embodiments, R is optionally substituted

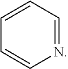

In some embodiments, R is

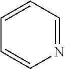

optionally substituted 1, 2, or 3 times by halogen or C₁₋₆ alkyl.

In some embodiments, each R is independently —CH₃, —CD₃, —CF₃, —CHF₂,

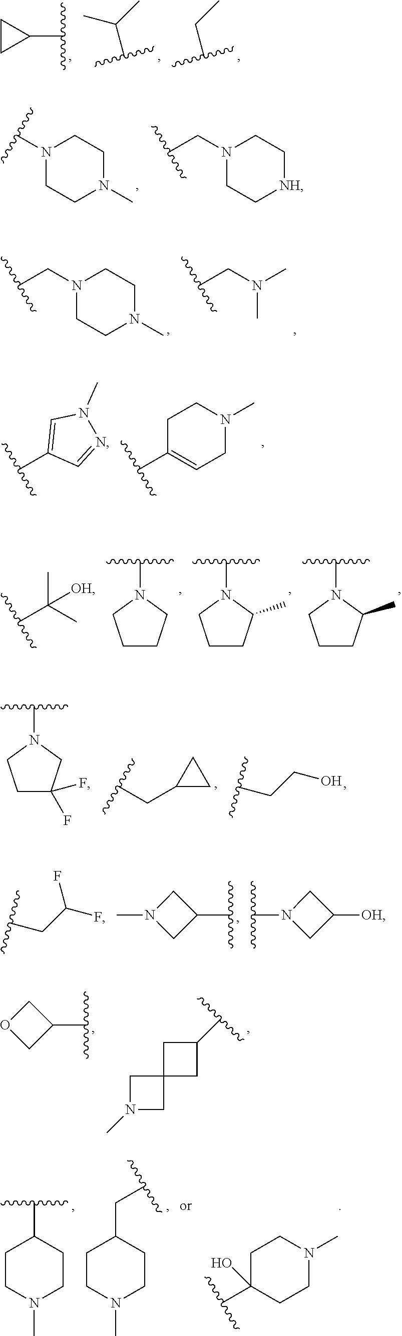

In some embodiments, R is

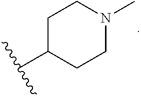 [SMILES: *C1CCN(C)CC1]

In some embodiments, R is

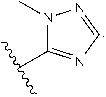 [SMILES: *c1ncnn1C]

In some embodiments, R is

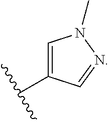 [SMILES: *c1cnn(C)c1]

In some embodiments, R is

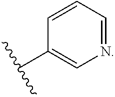 [SMILES: *c1cccnc1]

In some embodiments, R is

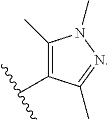 [SMILES: *c1c(C)nn(C)c1C]

In some embodiments, R is

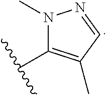 [SMILES: *c1c(C)cnn1C]

In some embodiments, R is

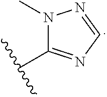 [SMILES: *c1ncnn1C]

In some embodiments, R is

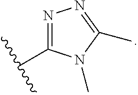 [SMILES: *c1nnc(C)n1C]

In some embodiments, each R is independently selected from those depicted in Table 1, below.

As defined generally above, n is 0, 1, 2 or 3.

In some embodiments, n is 0. In some embodiments, n is 1. In some embodiments, n is 2. In some embodiments, n is 3.

In some embodiments, n is selected from those depicted in Table 1, below.

As defined generally above, m is 0, 1, 2 or 3.

In some embodiments, m is 0. In some embodiments, m is 1. In some embodiments, m is 2. In some embodiments, m is 3.

In some embodiments, m is selected from those depicted in Table 1, below.

In some embodiments, the present invention provides a compound of Formula II or II′.

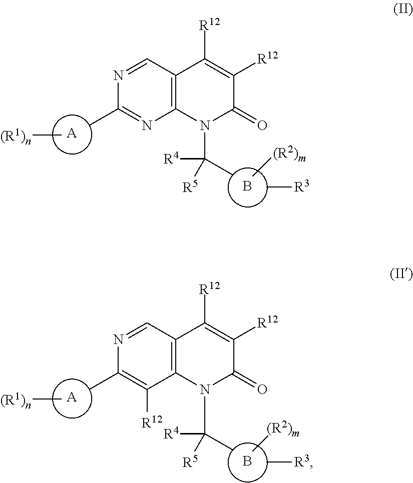

or a pharmaceutically acceptable salt thereof, wherein each variable is as defined above and as described in embodiments herein, both singly and in combination.

In some embodiments, the present invention provides a compound of Formula II-1 or II′-1:

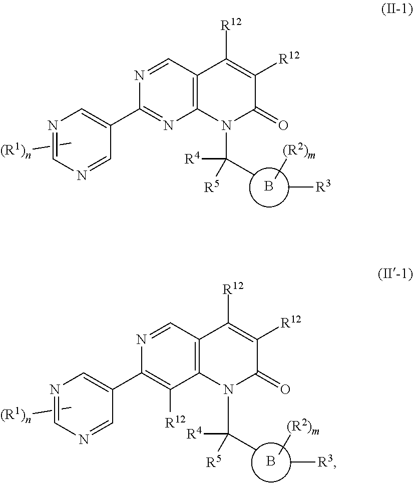

or a pharmaceutically acceptable salt thereof, wherein each variable is as defined above and as described in embodiments herein, both singly and in combination.

In some embodiments, the present invention provides a compound of Formula II-2 or II′-2.

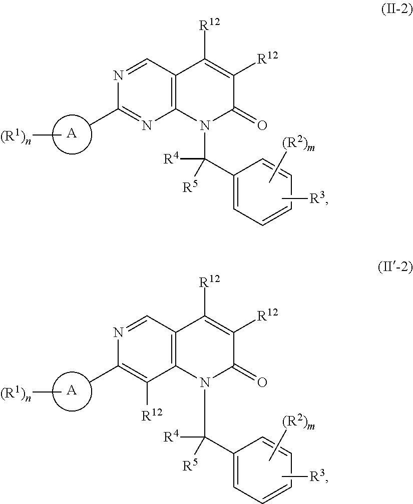

or a pharmaceutically acceptable salt thereof, wherein each variable is as defined above and as described in embodiments herein, both singly and in combination.

In some embodiments, the present invention provides a compound of Formula II-3 or II′-3:

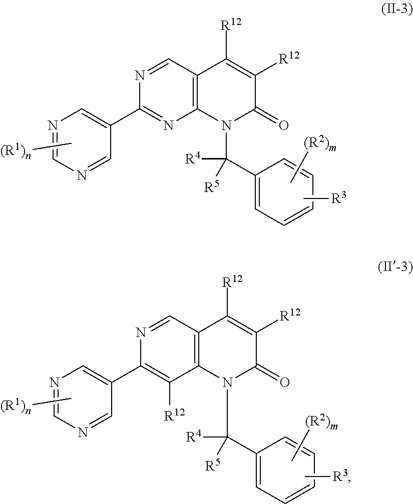

or a pharmaceutically acceptable salt thereof, wherein each variable is as defined above and as described in embodiments herein, both singly and in combination.

In some embodiments, the present invention provides a compound of Formula II-4 or II′-4:

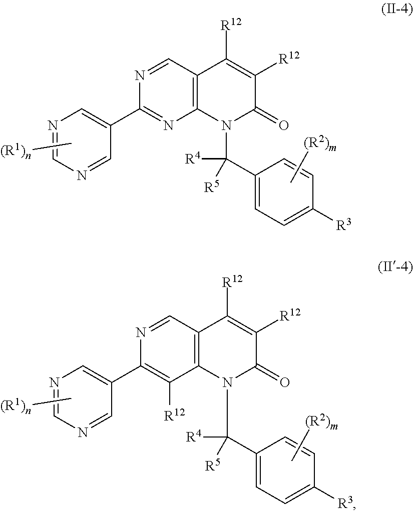

or a pharmaceutically acceptable salt thereof, wherein each variable is as defined above and as described in embodiments herein, both singly and in combination.

In some embodiments, the present invention provides a compound of Formula II-5.

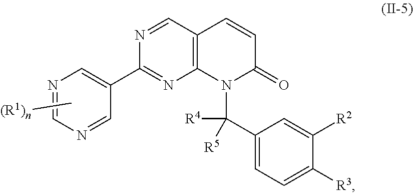

or a pharmaceutically acceptable salt thereof, wherein:
R² is —OR, wherein R is optionally substituted C₁₋₆ aliphatic; and
each remaining variable is as defined above and as described in embodiments herein, both singly and in combination.

In some embodiments, the present invention provides a compound of Formula II-6, 11-7, 11-8, or 11-9:

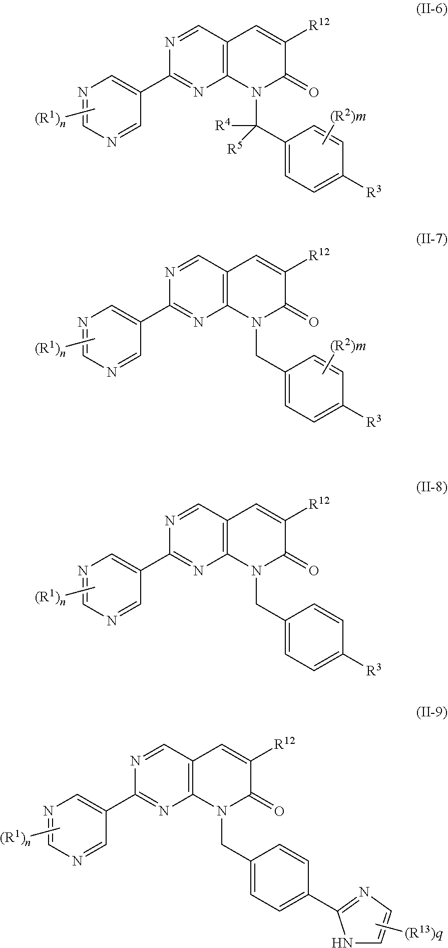

or a pharmaceutically acceptable salt thereof, wherein:
R¹² is an optionally substituted 3, 4, 5, 6, or 7 membered heterocyclic ring having 1, 2, 3, or 4 heteroatoms selected from N, O, or S, or an optionally substituted 5 or 6 membered heteroaromatic ring having 1, 2, 3, or 4 heteroatoms selected from N, O, and S; and
each remaining variable is as defined above and as described in embodiments herein, both singly and in combination.

In some embodiments, the present invention provides a compound of Formula II-6, 11-7, 11-8, or 11-9:

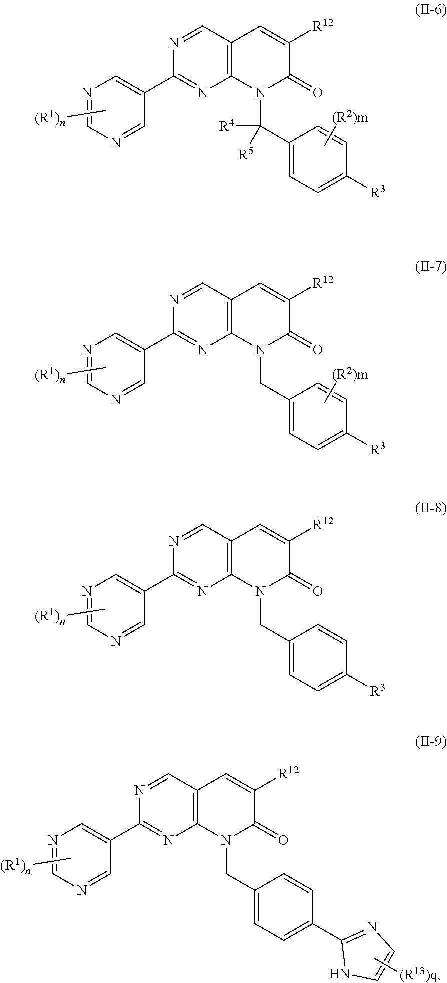

or a pharmaceutically acceptable salt thereof, wherein:
R¹² is an optionally substituted 5 or 6 membered heteroaromatic ring having 1, 2, 3, or 4 heteroatoms selected from N, O, and S; and each remaining variable is as defined above and as described in embodiments herein, both singly and in combination.

In some embodiments, the present invention provides a compound of Formula III or III′:

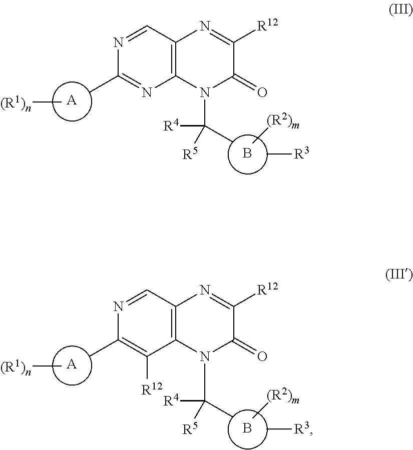

or a pharmaceutically acceptable salt thereof, wherein each variable is as defined above and as described in embodiments herein, both singly and in combination.

In some embodiments, the present invention provides a compound of Formula III-1 or III′-1:

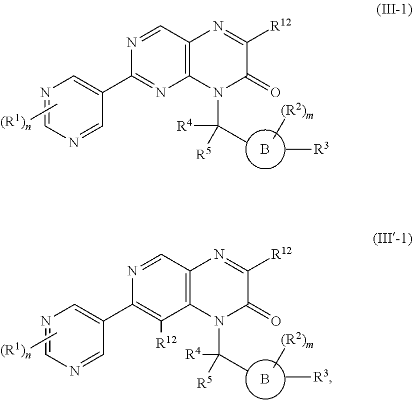

or a pharmaceutically acceptable salt thereof, wherein each variable is as defined above and as described in embodiments herein, both singly and in combination.

In some embodiments, the present invention provides a compound of Formula III-2 or III′-2:

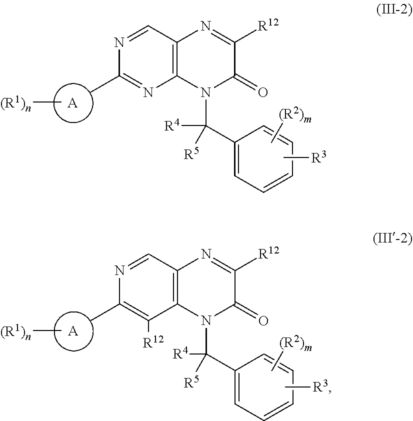

or a pharmaceutically acceptable salt thereof, wherein each variable is as defined above and as described in embodiments herein, both singly and in combination.

In some embodiments, the present invention provides a compound of Formula III-3 or III′-3:

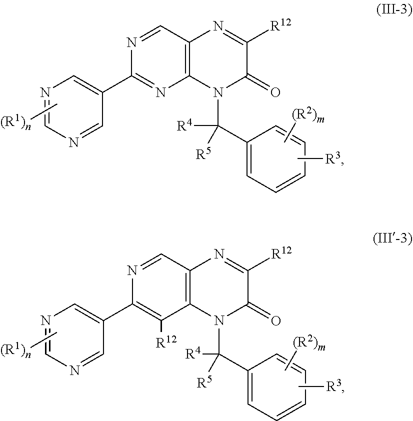

or a pharmaceutically acceptable salt thereof, wherein each variable is as defined above and as described in embodiments herein, both singly and in combination.

In some embodiments, the present invention provides a compound of Formula III-4 or III′-4:

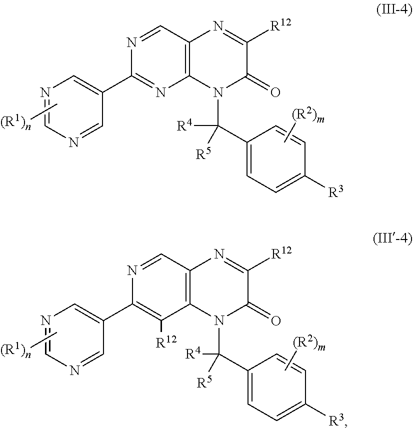

or a pharmaceutically acceptable salt thereof, wherein each variable is as defined above and as described in embodiments herein, both singly and in combination.

In some embodiments, the present invention provides a compound of Formula III-5.

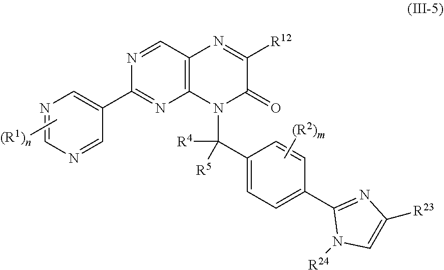

or a pharmaceutically acceptable salt thereof, wherein:
each R² is independently halogen or —OR, wherein R is optionally substituted C₁₋₆ aliphatic;
R²³ is optionally substituted C₁₋₆ aliphatic;
R²⁴ is optionally substituted C₁₋₆ aliphatic, or optionally substituted C₃-C₇ carbocyclic ring; and
each remaining variable is as defined above and as described in embodiments herein, both singly and in combination.

In some embodiments, the present invention provides a compound of Formula IV or IV′:

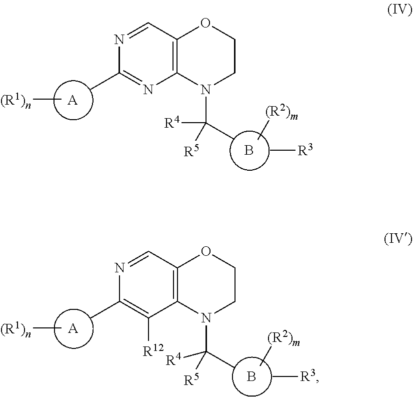

or a pharmaceutically acceptable salt thereof, wherein each variable is as defined above and as described in embodiments herein, both singly and in combination.

In some embodiments, the present invention provides a compound of Formula V or V′:

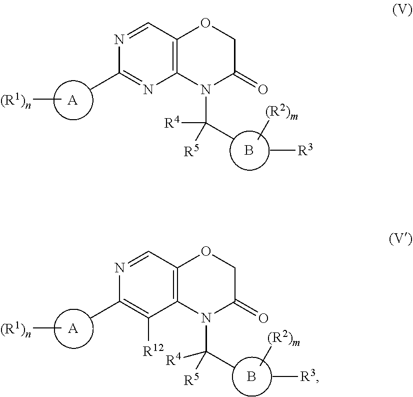

or a pharmaceutically acceptable salt thereof, wherein each variable is as defined above and as described in embodiments herein, both singly and in combination.

In some embodiments, the present invention provides a compound of Formula VI or VI′:

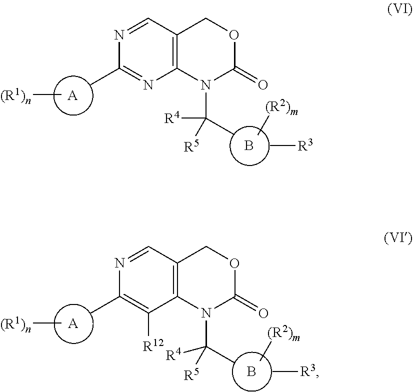

or a pharmaceutically acceptable salt thereof, wherein each variable is as defined above and as described in embodiments herein, both singly and in combination.

In some embodiments, the present invention provides a compound of Formula VII or VII′:

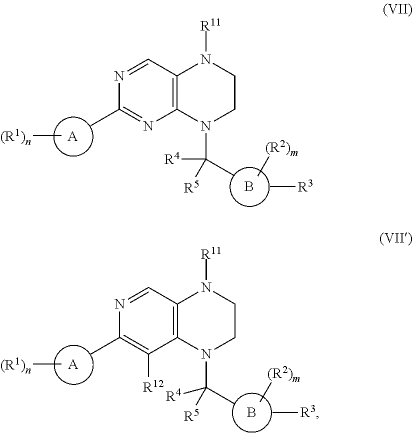

or a pharmaceutically acceptable salt thereof, wherein each variable is as defined above and as described in embodiments herein, both singly and in combination.

In some embodiments, the present invention provides a compound of Formula VIII or VIII′:

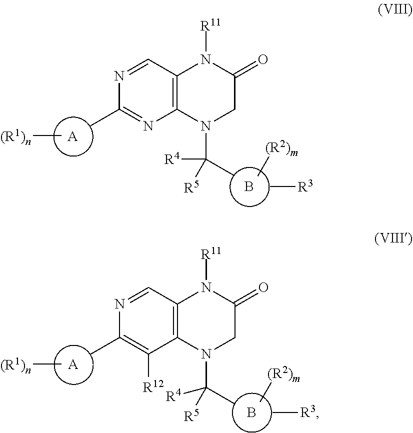

or a pharmaceutically acceptable salt thereof, wherein each variable is as defined above and as described in embodiments herein, both singly and in combination.

In some embodiments, the present invention provides a compound of Formula IX or IX′:

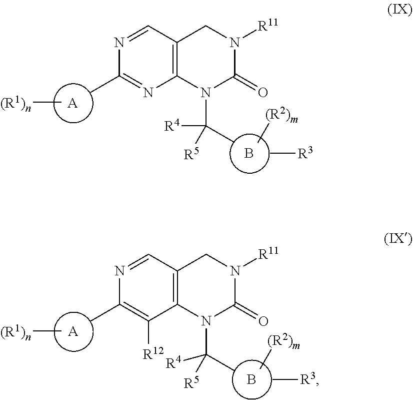

or a pharmaceutically acceptable salt thereof, wherein each variable is as defined above and as described in embodiments herein, both singly and in combination.

In some embodiments, the present invention provides a compound of Formula X or X′:

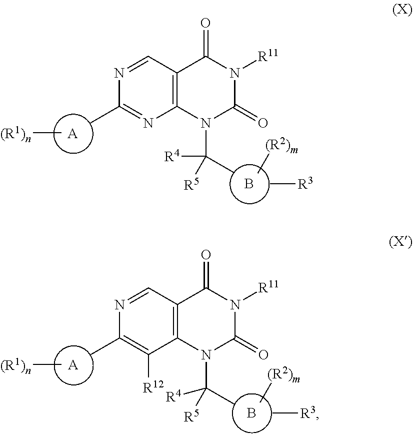

or a pharmaceutically acceptable salt thereof, wherein each variable is as defined above and as described in embodiments herein, both singly and in combination.

In some embodiments, the present invention provides a compound of Formula XI or XI′:

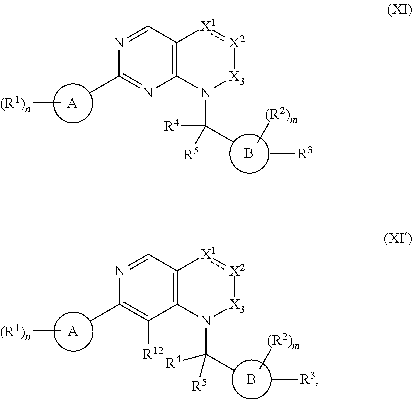

or a pharmaceutically acceptable salt thereof, wherein each variable is as defined above and as described in embodiments herein, both singly and in combination.

In some embodiments, the present invention provides a compound of Formulae XI-1 to XI-4, or XI′-1 to XI′-4:

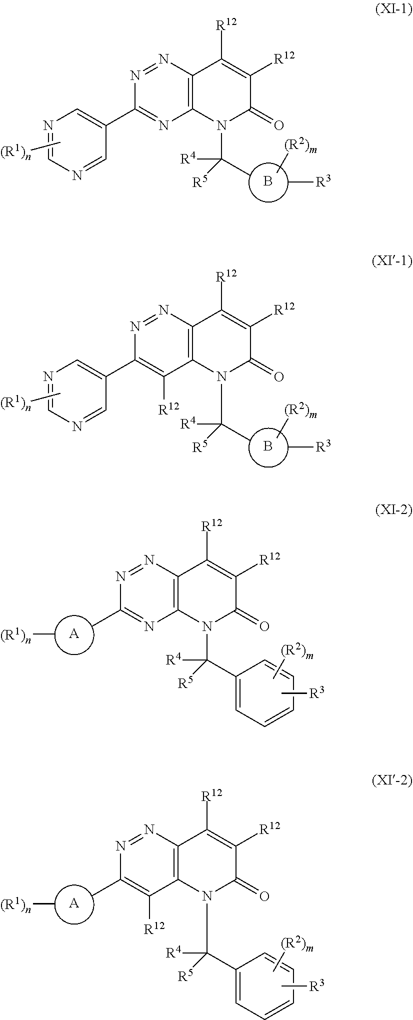

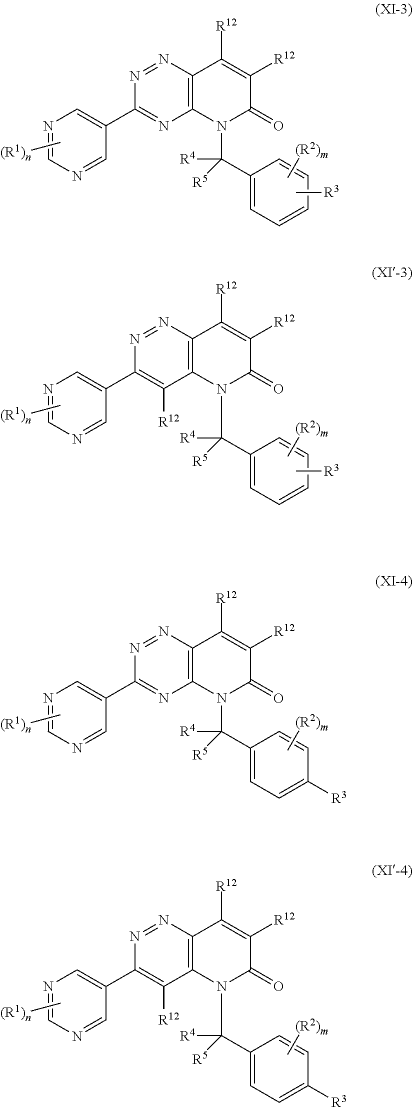

or a pharmaceutically acceptable salt thereof, wherein each variable is as defined above and as described in embodiments herein, both singly and in combination.

In some embodiments, the present invention provides a compound of Formula XII or XII′:

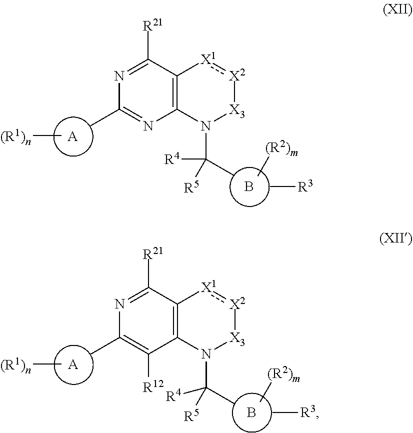

or a pharmaceutically acceptable salt thereof, wherein R²¹ is optionally substituted C₁₋₆ aliphatic, and each remaining variable is as defined above and as described in embodiments herein, both singly and in combination.

In some embodiments, R²¹ is unsubstituted C₁₋₆ aliphatic. In some embodiments, R²¹ is unsubstituted C₁₋₆ alkyl. In some embodiments, R²¹ is C₁₋₆ aliphatic substituted 1, 2, 3, 4, 5, or 6 times by halogen or —OH. In some embodiments, R²¹ is C₁₋₆ alkyl substituted 1, 2, 3, 4, 5, or 6 times by halogen or —OH. In some embodiments, R²¹ is —CH₃, —CD₃,

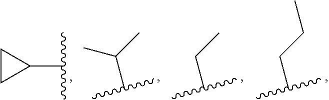

—CH₂F, —CHF₂, or —CF₃.

In some embodiments, the present invention provides a compound of Formulae XII-1 to XII-5, or XII′-1 to XII′-5:

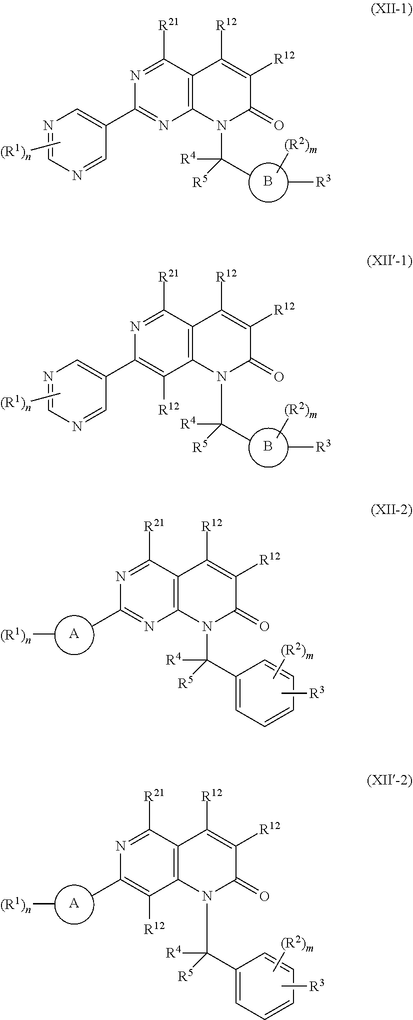

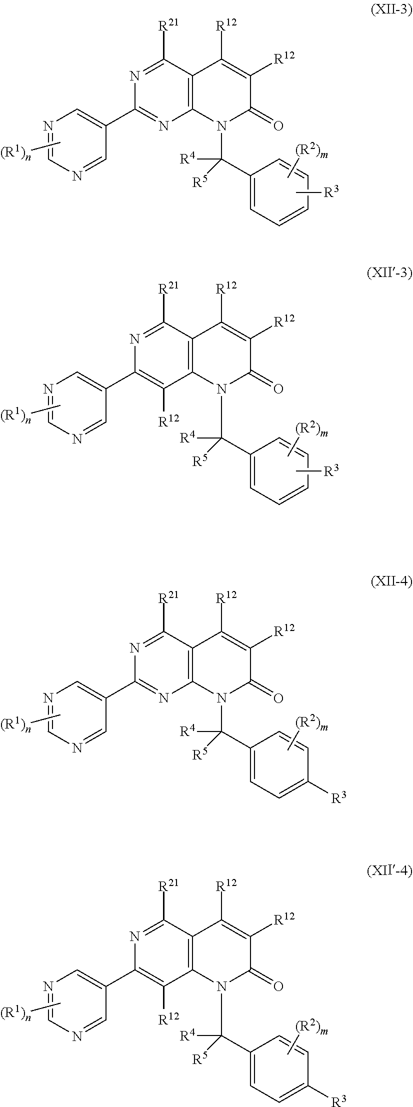

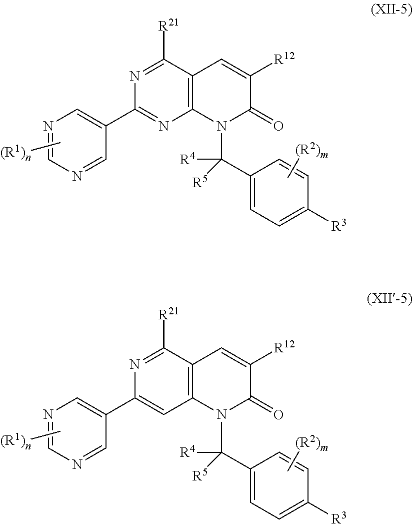

or a pharmaceutically acceptable salt thereof, wherein R²¹ is optionally substituted C₁-6 aliphatic, and each remaining variable is as defined above and as described in embodiments herein, both singly and in combination.

In some embodiments, the present invention provides a compound of Formula XII-6XII-7 or XII-8 or XII-9.

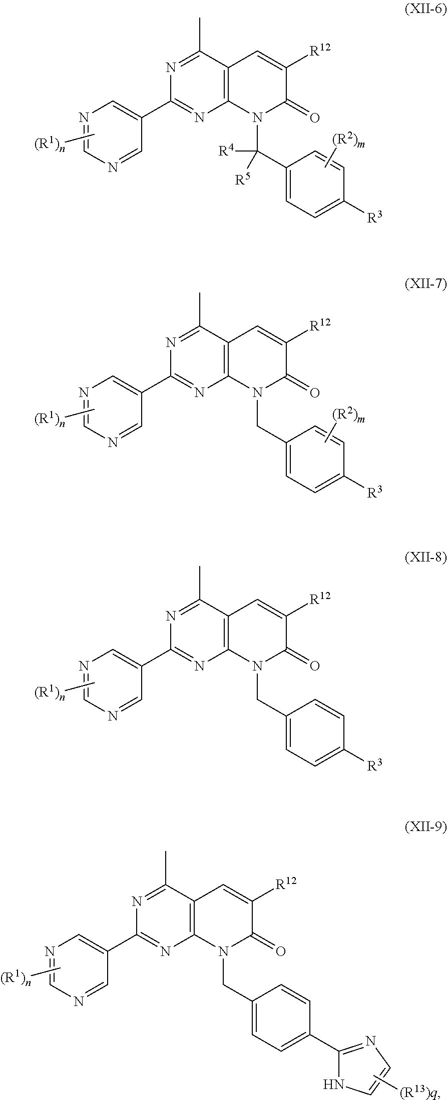

or a pharmaceutically acceptable salt thereof, wherein:
R¹² is an optionally substituted 3, 4, 5, 6, or 7 membered heterocyclic ring having 1, 2, 3, or 4 heteroatoms selected from N, O, or S, or an optionally substituted 5 or 6 membered heteroaromatic ring having 1, 2, 3, or 4 heteroatoms selected from N, O, and S; and
each remaining variable is as defined above and as described in embodiments herein, both singly and in combination.

In some embodiments, the present invention provides a compound of Formula XII-XII-11, XII-12, XII-13, XII-14, or XII-15:

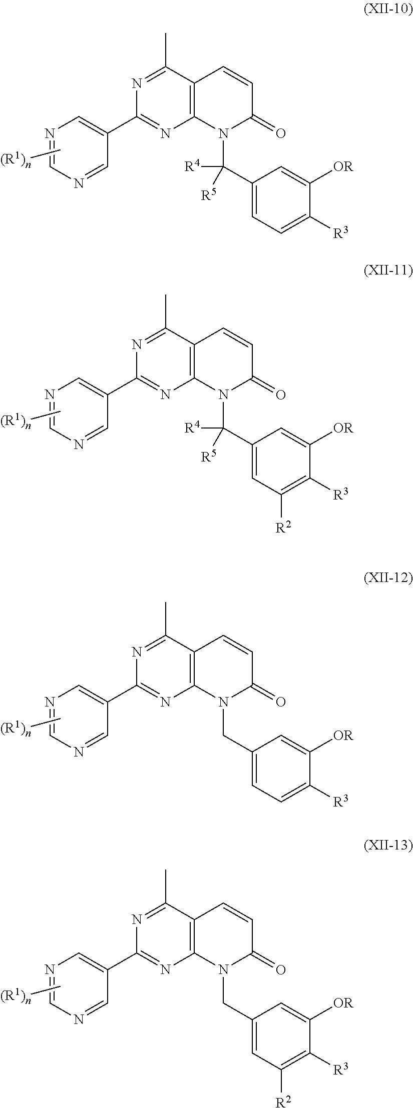

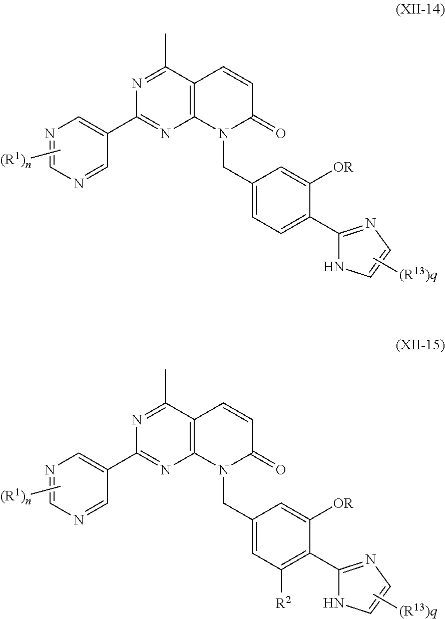

or a pharmaceutically acceptable salt thereof, wherein each variable is as defined above and as described in embodiments herein, both singly and in combination.

In some embodiments, the present invention provides a compound of Formula XIII or XIII′:

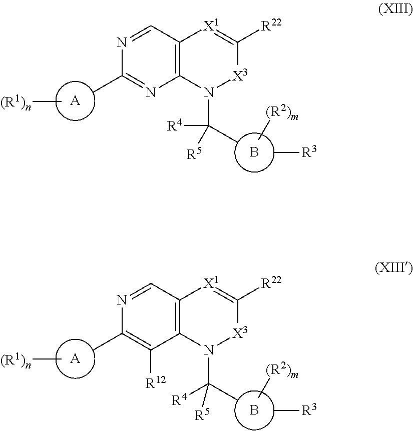

or a pharmaceutically acceptable salt thereof, wherein:
R²² is an optionally substituted ring selected from a phenyl ring and a 5-6 membered heteroaromatic ring having 1-4 heteroatoms selected from N, O, and S; and
each remaining variable is as defined above and as described in embodiments herein, both singly and in combination.

In some embodiments, the present invention provides a compound of Formulae XIII-1 to XIII-5, or XIII′-1 to XIII′-5:

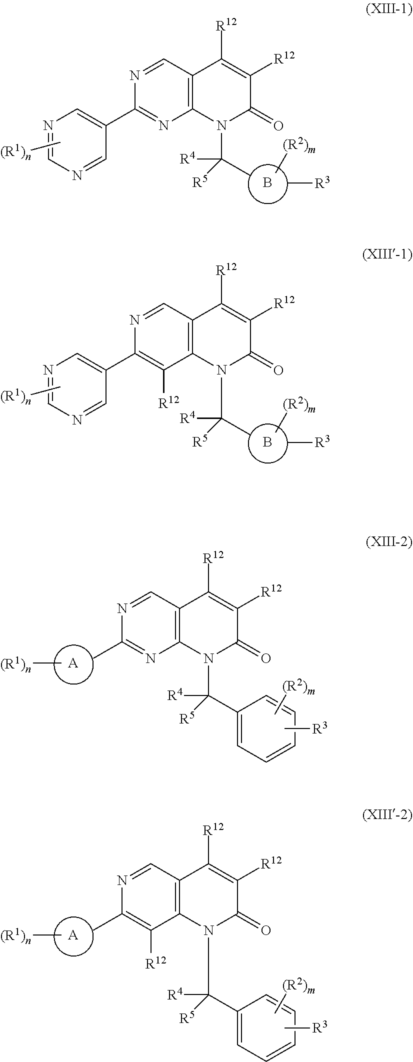

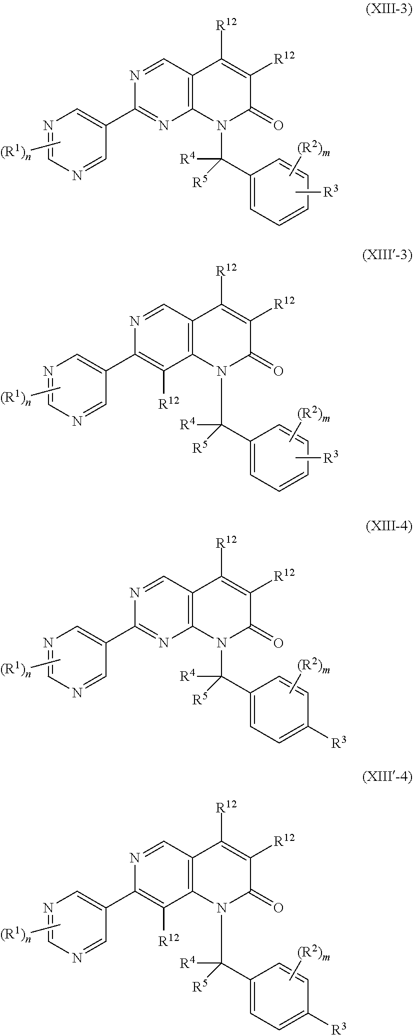

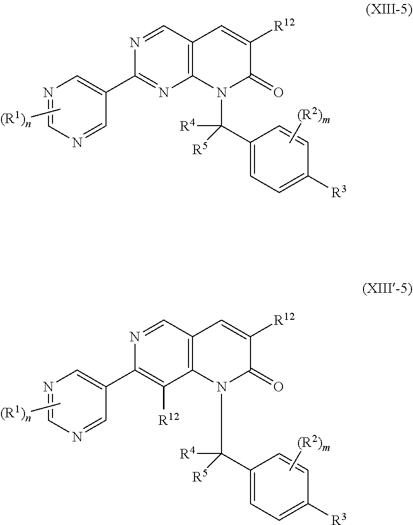

or a pharmaceutically acceptable salt thereof, wherein:
R²² is an optionally substituted ring selected from a phenyl ring and a 5-6 membered heteroaromatic ring having 1-4 heteroatoms selected from N, O, and S; and
each remaining variable is as defined above and as described in embodiments herein, both singly and in combination.

In some embodiments, R²² is optionally substituted phenyl. In some embodiments, R²² is phenyl optionally substituted 1, 2, or 3 times by halogen or C₁₋₆ alkyl. In some embodiments, R²² is optionally substituted 5 or 6 membered heteroaromatic ring having 1, 2, 3, or 4 heteroatoms selected from N, O, and S. In some embodiments, R²² is 5 or 6 membered heteroaromatic ring having 1, 2, 3, or 4 heteroatoms selected from N, O, and S, optionally substituted 1, 2, or 3 times by halogen or C₁₋₆ alkyl. In some embodiments, R²² is optionally substituted

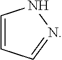

In some embodiments, R²² is

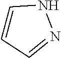

optionally substituted 1, 2, or 3 times by halogen or C₁₋₆ alkyl. In some embodiments, R²² is optionally substituted

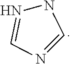

In some embodiments, R²² is

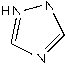

optionally substituted 1, 2, or 3 times by halogen or C₁₋₆ alkyl. In some embodiments, R²² is optionally substituted

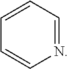

In some embodiments, R²² is

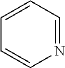

optionally substituted 1, 2, or 3 times by halogen or C₁₋₆ alkyl.

In some embodiments, R²² is

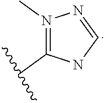

In some embodiments, R²² is

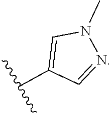 [SMILES: *c1cnn(C)c1]

In some embodiments, R²² is

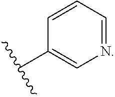 [SMILES: *c1cccnc1]

In some embodiments, R²² is

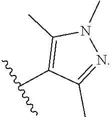 [SMILES: *c1c(C)nn(C)c1C]

In some embodiments, R²² is

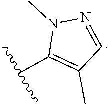 [SMILES: *c1c(C)cnn1C]

In some embodiments, R²² is

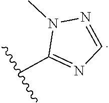 [SMILES: *c1ncnn1C]

In some embodiments, R²² is

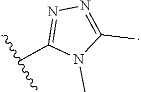 [SMILES: *c1nnc(C)n1C]

In some embodiments, the present invention provides a compound of Formula XIV or XIV′:

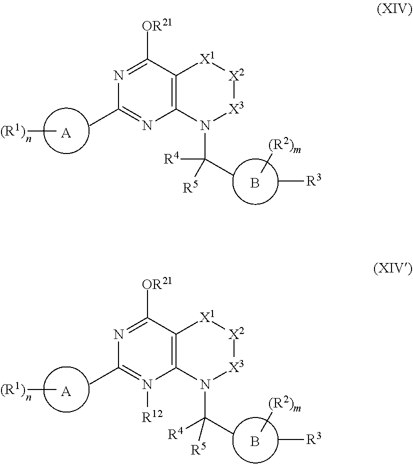

, or a pharmaceutically acceptable salt thereof, wherein R²¹ is optionally substituted C₁₋₆ aliphatic, and each remaining variable is as defined above and as described in embodiments herein, both singly and in combination.

In some embodiments, the present invention provides a compound of Formulae XIV-1 to XIV-5, or XIV′-1 to XIV′-5:

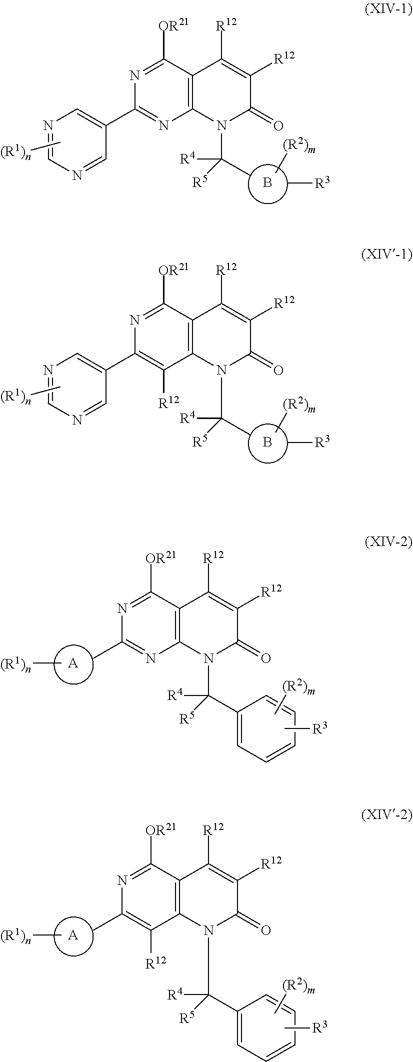

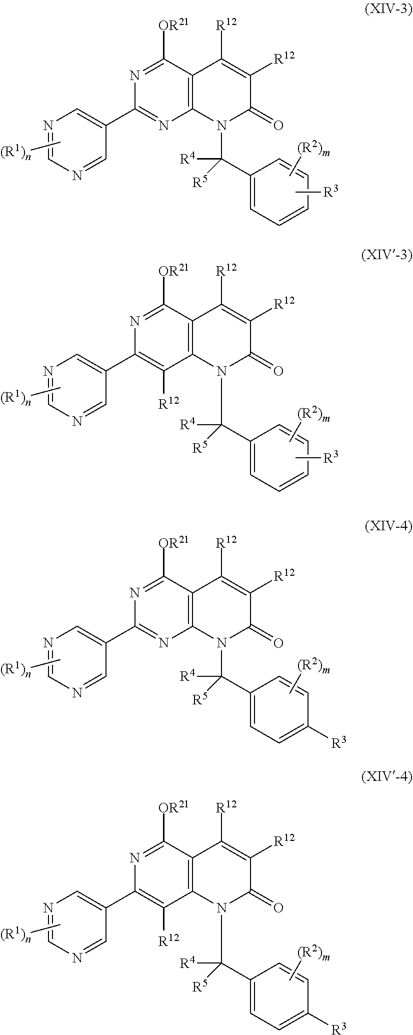

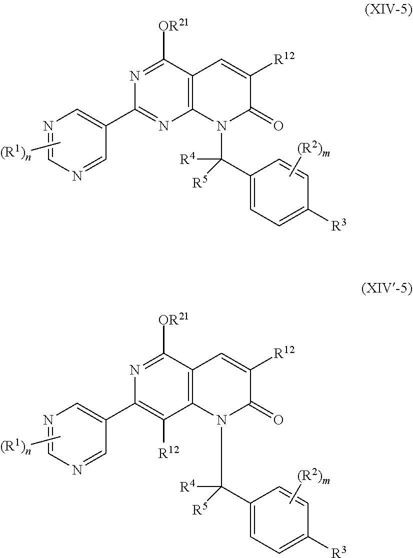

or a pharmaceutically acceptable salt thereof, wherein R²¹ is optionally substituted C₁-6 aliphatic, and each remaining variable is as defined above and as described in embodiments herein, both singly and in combination.

In some embodiments, the present invention provides a compound of Formula XIV-6:

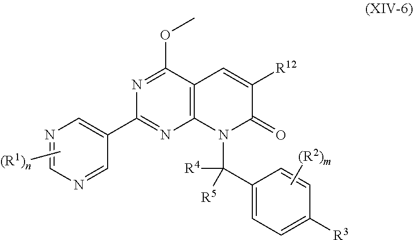

or a pharmaceutically acceptable salt thereof, wherein:
R¹² is hydrogen, an optionally substituted 3, 4, 5, 6, or 7 membered heterocyclic ring having 1, 2, 3, or 4 heteroatoms selected from N, O, or S, or an optionally substituted 5 or 6 membered heteroaromatic ring having 1, 2, 3, or 4 heteroatoms selected from N, O, and S; and
each remaining variable is as defined above and as described in embodiments herein, both singly and in combination.

Exemplary compounds of the invention are set forth in Table 1, below.

In some embodiments, the present invention provides a compound set forth in Table 1, below, or a pharmaceutically acceptable salt thereof.

TABLE 1

| | |
|---|---|
| 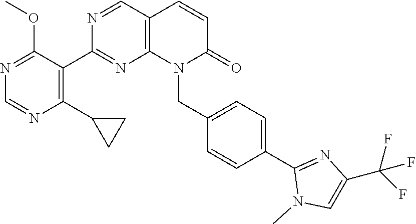 [SMILES: COc1ncnc(C2CC2)c1-c1ncc2ccc(=O)n(Cc3ccc(-c4nc(C(F)(F)F)cn4C)cc3)c2n1] | I-1 |
| 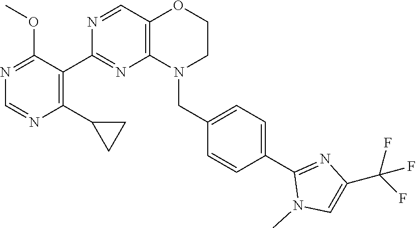 [SMILES: COc1ncnc(C2CC2)c1-c1ncc2c(n1)N(Cc1ccc(-c3nc(C(F)(F)F)cn3C)cc1)CCO2] | I-2 |
| 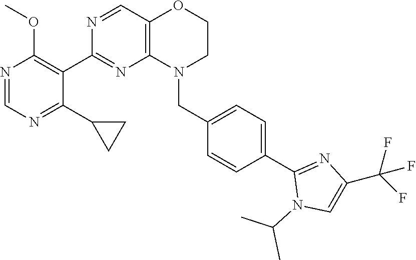 [SMILES: COc1ncnc(C2CC2)c1-c1ncc2c(n1)N(Cc1ccc(-c3nc(C(F)(F)F)cn3C(C)C)cc1)CCO2] | I-3 |
| 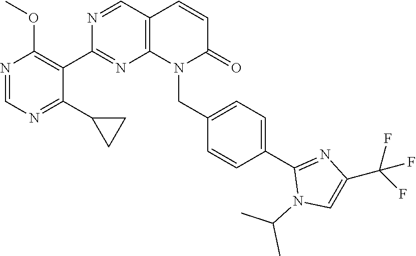 [SMILES: COc1ncnc(C2CC2)c1-c1ncc2ccc(=O)n(Cc3ccc(-c4nc(C(F)(F)F)cn4C(C)C)cc3)c2n1] | I-4 |
| 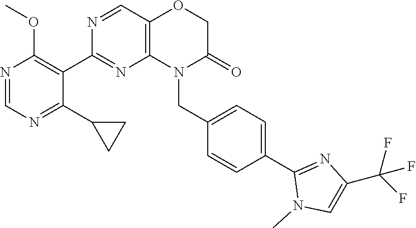 [SMILES: COc1ncnc(C2CC2)c1-c1ncc2c(n1)N(Cc1ccc(-c3nc(C(F)(F)F)cn3C)cc1)C(=O)CO2] | I-5 |
| 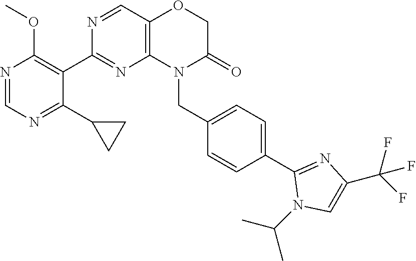 [SMILES: COc1ncnc(C2CC2)c1-c1ncc2c(n1)N(Cc1ccc(-c3nc(C(F)(F)F)cn3C(C)C)cc1)C(=O)CO2] | I-6 |
| 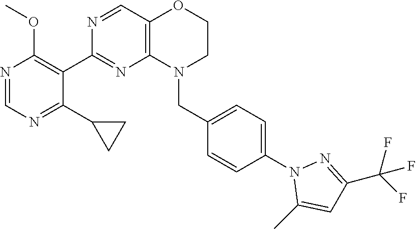 [SMILES: COc1ncnc(C2CC2)c1-c1ncc2c(n1)N(Cc1ccc(-n3nc(C(F)(F)F)cc3C)cc1)CCO2] | I-7 |
| 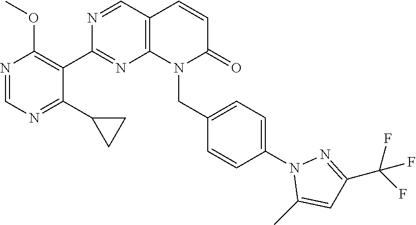 [SMILES: COc1ncnc(C2CC2)c1-c1ncc2ccc(=O)n(Cc3ccc(-n4nc(C(F)(F)F)cc4C)cc3)c2n1] | I-8 |
| 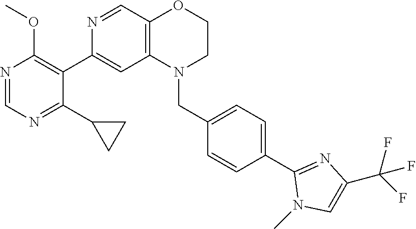 [SMILES: COc1ncnc(C2CC2)c1-c1cc2c(cn1)OCCN2Cc1ccc(-c2nc(C(F)(F)F)cn2C)cc1] | I-9 |
| 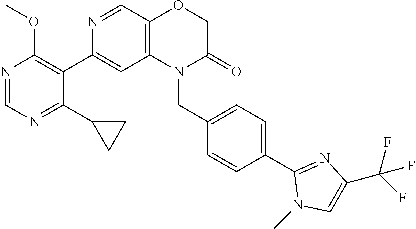 [SMILES: COc1ncnc(C2CC2)c1-c1cc2c(cn1)OCC(=O)N2Cc1ccc(-c2nc(C(F)(F)F)cn2C)cc1] | I-10 |
| 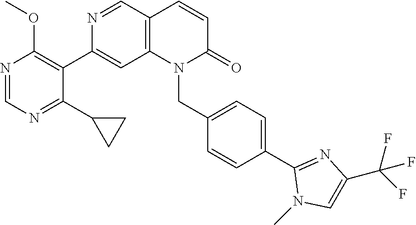 [SMILES: COc1ncnc(C2CC2)c1-c1cc2c(ccc(=O)n2Cc2ccc(-c3nc(C(F)(F)F)cn3C)cc2)cn1] | I-11 |
| 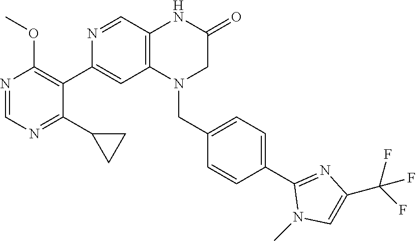 [SMILES: COc1ncnc(C2CC2)c1-c1cc2c(cn1)NC(=O)CN2Cc1ccc(-c2nc(C(F)(F)F)cn2C)cc1] | I-12 |
| 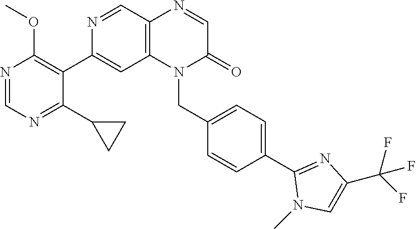 [SMILES: COc1ncnc(C2CC2)c1-c1cc2c(cn1)ncc(=O)n2Cc1ccc(-c2nc(C(F)(F)F)cn2C)cc1] | I-13 |
| 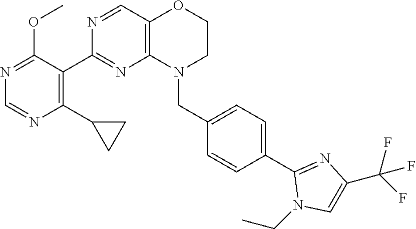 [SMILES: CCn1cc(C(F)(F)F)nc1-c1ccc(CN2CCOc3cnc(-c4c(OC)ncnc4C4CC4)nc32)cc1] | I-14 |
| 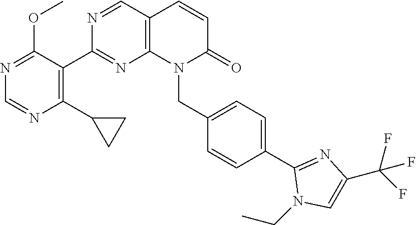 [SMILES: CCn1cc(C(F)(F)F)nc1-c1ccc(Cn2c(=O)ccc3cnc(-c4c(OC)ncnc4C4CC4)nc32)cc1] | I-15 |
| 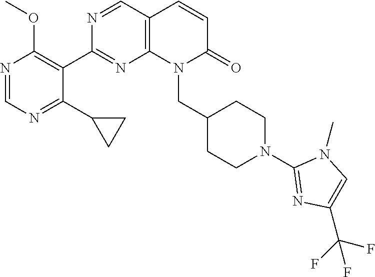 [SMILES: COc1ncnc(C2CC2)c1-c1ncc2ccc(=O)n(CC3CCN(c4nc(C(F)(F)F)cn4C)CC3)c2n1] | I-16 |
| 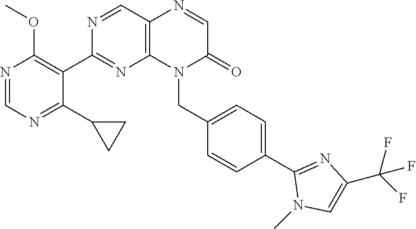 [SMILES: COc1ncnc(C2CC2)c1-c1ncc2ncc(=O)n(Cc3ccc(-c4nc(C(F)(F)F)cn4C)cc3)c2n1] | I-17 |
| 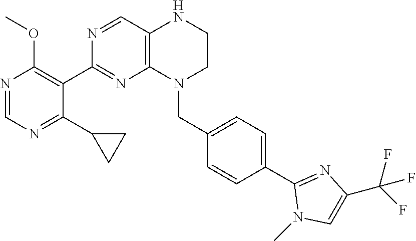 [SMILES: COc1ncnc(C2CC2)c1-c1ncc2c(n1)N(Cc1ccc(-c3nc(C(F)(F)F)cn3C)cc1)CCN2] | I-18 |
| 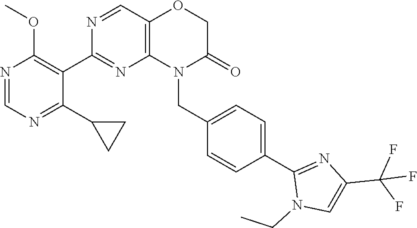 [SMILES: CCn1cc(C(F)(F)F)nc1-c1ccc(CN2C(=O)COc3cnc(-c4c(OC)ncnc4C4CC4)nc32)cc1] | I-19 |
| 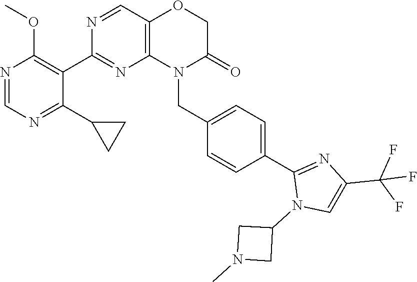 [SMILES: COc1ncnc(C2CC2)c1-c1ncc2c(n1)N(Cc1ccc(-c3nc(C(F)(F)F)cn3C3CN(C)C3)cc1)C(=O)CO2] | I-20 |
| 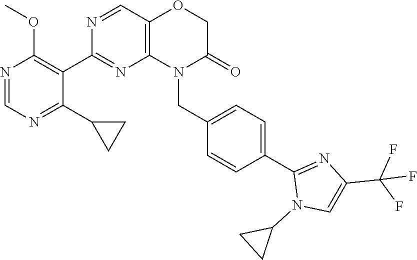 [SMILES: COc1ncnc(C2CC2)c1-c1ncc2c(n1)N(Cc1ccc(-c3nc(C(F)(F)F)cn3C3CC3)cc1)C(=O)CO2] | I-21 |
| 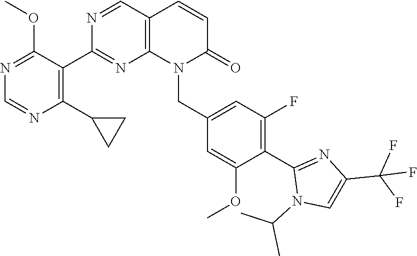 [SMILES: COc1cc(Cn2c(=O)ccc3cnc(-c4c(OC)ncnc4C4CC4)nc32)cc(F)c1-c1nc(C(F)(F)F)cn1C(C)C] | I-22 |
| 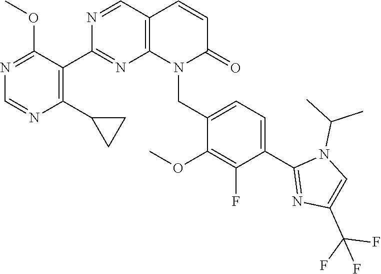 [SMILES: COc1ncnc(C2CC2)c1-c1ncc2ccc(=O)n(Cc3ccc(-c4nc(C(F)(F)F)cn4C(C)C)c(F)c3OC)c2n1] | I-23 |
| 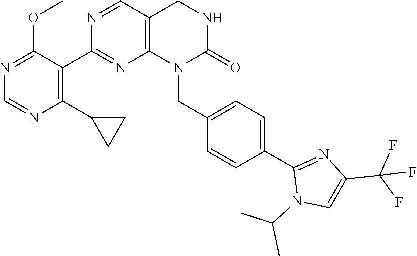 [SMILES: COc1ncnc(C2CC2)c1-c1ncc2c(n1)N(Cc1ccc(-c3nc(C(F)(F)F)cn3C(C)C)cc1)C(=O)NC2] | I-24 |
| 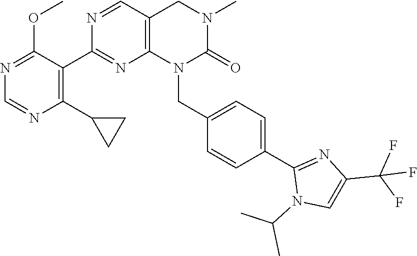 [SMILES: COc1ncnc(C2CC2)c1-c1ncc2c(n1)N(Cc1ccc(-c3nc(C(F)(F)F)cn3C(C)C)cc1)C(=O)N(C)C2] | I-25 |
| 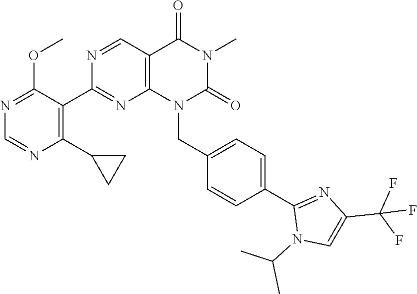 [SMILES: COc1ncnc(C2CC2)c1-c1ncc2c(=O)n(C)c(=O)n(Cc3ccc(-c4nc(C(F)(F)F)cn4C(C)C)cc3)c2n1] | I-26 |
| 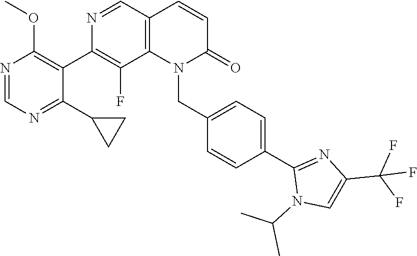 [SMILES: COc1ncnc(C2CC2)c1-c1ncc2ccc(=O)n(Cc3ccc(-c4nc(C(F)(F)F)cn4C(C)C)cc3)c2c1F] | I-27 |
| 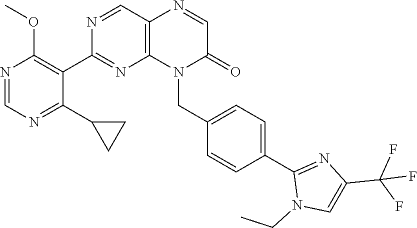 [SMILES: CCn1cc(C(F)(F)F)nc1-c1ccc(Cn2c(=O)cnc3cnc(-c4c(OC)ncnc4C4CC4)nc32)cc1] | I-28 |
| 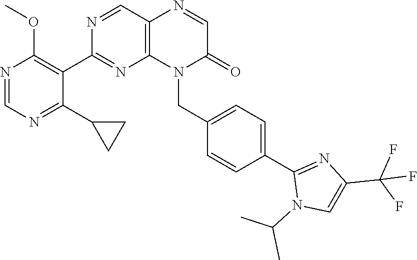 [SMILES: COc1ncnc(C2CC2)c1-c1ncc2ncc(=O)n(Cc3ccc(-c4nc(C(F)(F)F)cn4C(C)C)cc3)c2n1] | I-29 |
| 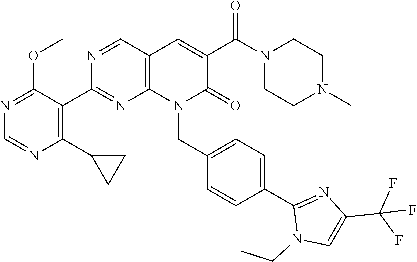 [SMILES: CCn1cc(C(F)(F)F)nc1-c1ccc(Cn2c(=O)c(C(=O)N3CCN(C)CC3)cc3cnc(-c4c(OC)ncnc4C4CC4)nc32)cc1] | I-30 |
| 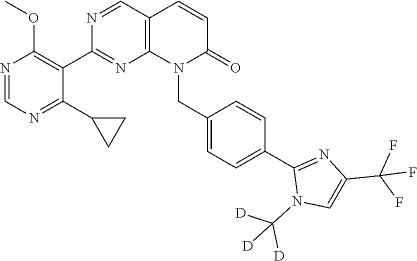 [SMILES: [2H]C([2H])([2H])n1cc(C(F)(F)F)nc1-c1ccc(Cn2c(=O)ccc3cnc(-c4c(OC)ncnc4C4CC4)nc32)cc1] | I-31 |
| 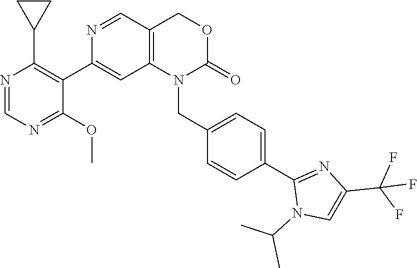 [SMILES: COc1ncnc(C2CC2)c1-c1cc2c(cn1)COC(=O)N2Cc1ccc(-c2nc(C(F)(F)F)cn2C(C)C)cc1] | I-32 |
| 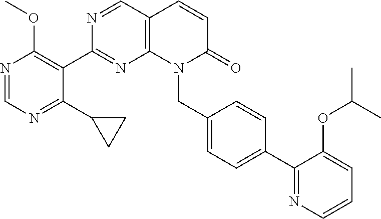 [SMILES: COc1ncnc(C2CC2)c1-c1ncc2ccc(=O)n(Cc3ccc(-c4ncccc4OC(C)C)cc3)c2n1] | I-33 |
| 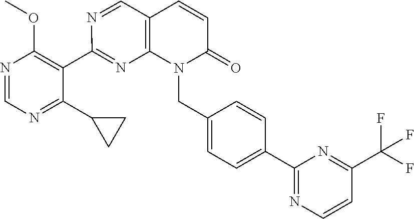 [SMILES: COc1ncnc(C2CC2)c1-c1ncc2ccc(=O)n(Cc3ccc(-c4nccc(C(F)(F)F)n4)cc3)c2n1] | I-34 |
| 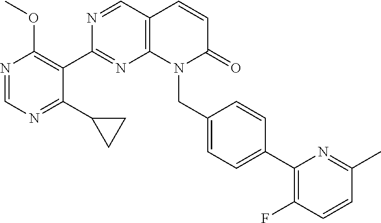 [SMILES: COc1ncnc(C2CC2)c1-c1ncc2ccc(=O)n(Cc3ccc(-c4nc(C)ccc4F)cc3)c2n1] | I-35 |
| 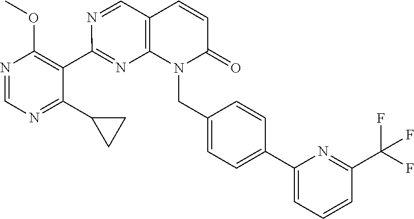 [SMILES: COc1ncnc(C2CC2)c1-c1ncc2ccc(=O)n(Cc3ccc(-c4cccc(C(F)(F)F)n4)cc3)c2n1] | I-36 |
| 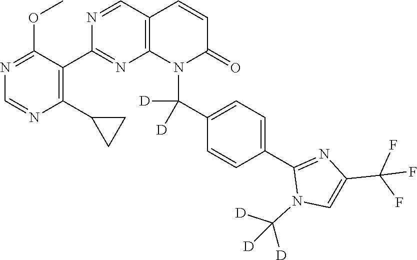 [SMILES: [2H]C([2H])([2H])n1cc(C(F)(F)F)nc1-c1ccc(C([2H])([2H])n2c(=O)ccc3cnc(-c4c(OC)ncnc4C4CC4)nc32)cc1] | I-37 |
| 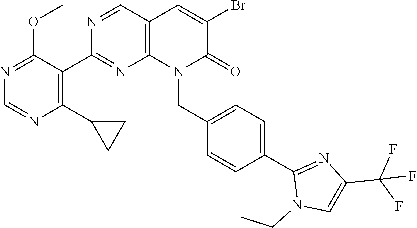 [SMILES: CCn1cc(C(F)(F)F)nc1-c1ccc(Cn2c(=O)c(Br)cc3cnc(-c4c(OC)ncnc4C4CC4)nc32)cc1] | I-38 |
| 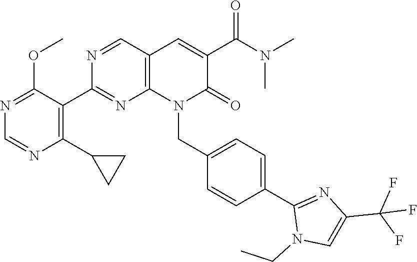 [SMILES: CCn1cc(C(F)(F)F)nc1-c1ccc(Cn2c(=O)c(C(=O)N(C)C)cc3cnc(-c4c(OC)ncnc4C4CC4)nc32)cc1] | I-39 |
| 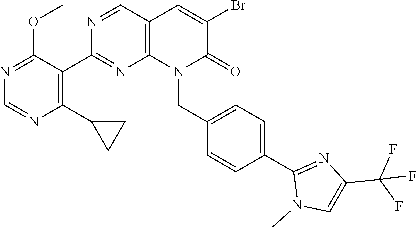 [SMILES: COc1ncnc(C2CC2)c1-c1ncc2cc(Br)c(=O)n(Cc3ccc(-c4nc(C(F)(F)F)cn4C)cc3)c2n1] | I-40 |
| 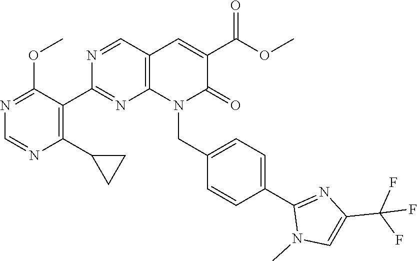 | I-41 |
| 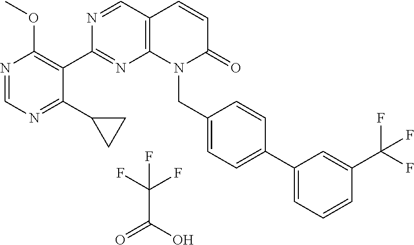 | I-42 |
| 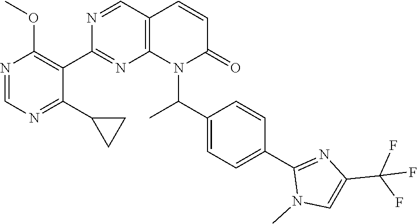 [SMILES: COc1ncnc(C2CC2)c1-c1ncc2ccc(=O)n(C(C)c3ccc(-c4nc(C(F)(F)F)cn4C)cc3)c2n1] | I-43 |
| 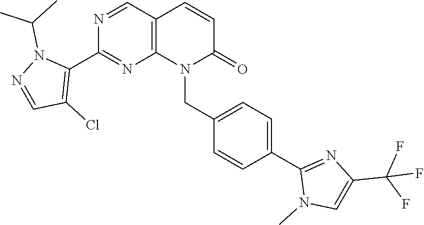 [SMILES: CC(C)n1ncc(Cl)c1-c1ncc2ccc(=O)n(Cc3ccc(-c4nc(C(F)(F)F)cn4C)cc3)c2n1] | I-44 |
| 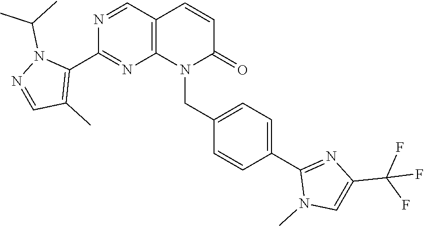 [SMILES: Cc1cnn(C(C)C)c1-c1ncc2ccc(=O)n(Cc3ccc(-c4nc(C(F)(F)F)cn4C)cc3)c2n1] | I-45 |
| 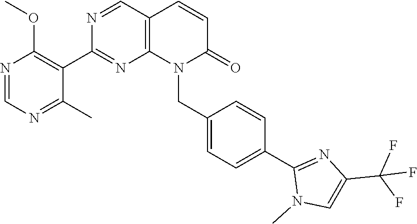 [SMILES: COc1ncnc(C)c1-c1ncc2ccc(=O)n(Cc3ccc(-c4nc(C(F)(F)F)cn4C)cc3)c2n1] | I-46 |
| 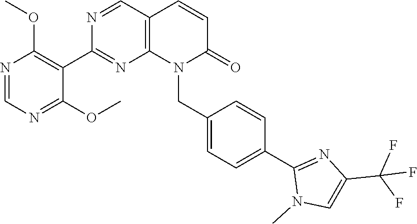 [SMILES: COc1ncnc(OC)c1-c1ncc2ccc(=O)n(Cc3ccc(-c4nc(C(F)(F)F)cn4C)cc3)c2n1] | I-47 |
| 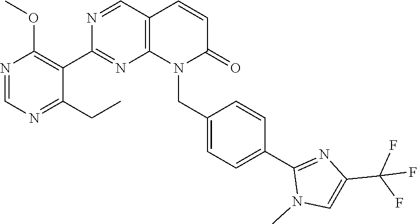 [SMILES: CCc1ncnc(OC)c1-c1ncc2ccc(=O)n(Cc3ccc(-c4nc(C(F)(F)F)cn4C)cc3)c2n1] | I-48 |
| 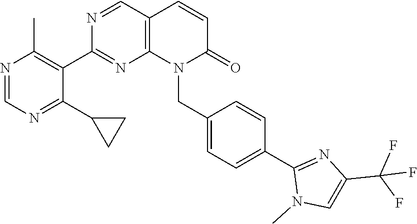 [SMILES: Cc1ncnc(C2CC2)c1-c1ncc2ccc(=O)n(Cc3ccc(-c4nc(C(F)(F)F)cn4C)cc3)c2n1] | I-49 |
| 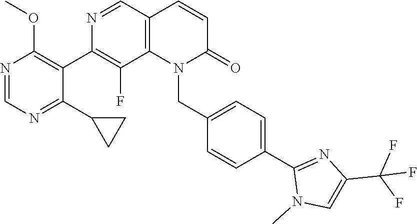 [SMILES: COc1ncnc(C2CC2)c1-c1ncc2ccc(=O)n(Cc3ccc(-c4nc(C(F)(F)F)cn4C)cc3)c2c1F] | I-50 |
| 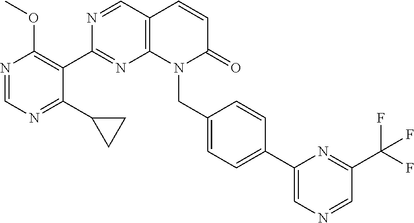 | I-51 |
| 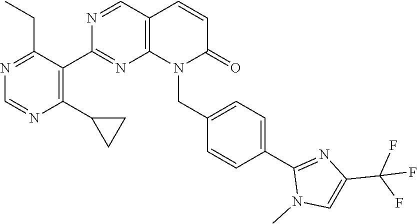 [SMILES: CCc1ncnc(C2CC2)c1-c1ncc2ccc(=O)n(Cc3ccc(-c4nc(C(F)(F)F)cn4C)cc3)c2n1] | I-52 |
| 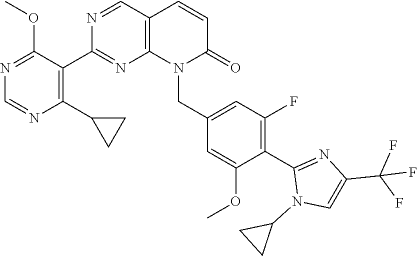 [SMILES: COc1cc(Cn2c(=O)ccc3cnc(-c4c(OC)ncnc4C4CC4)nc32)cc(F)c1-c1nc(C(F)(F)F)cn1C1CC1] | I-53 |
| 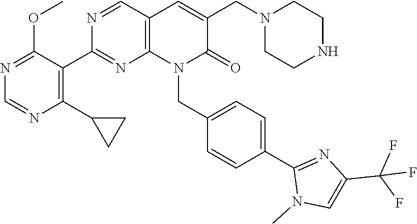 [SMILES: COc1ncnc(C2CC2)c1-c1ncc2cc(CN3CCNCC3)c(=O)n(Cc3ccc(-c4nc(C(F)(F)F)cn4C)cc3)c2n1] | I-54 |
| 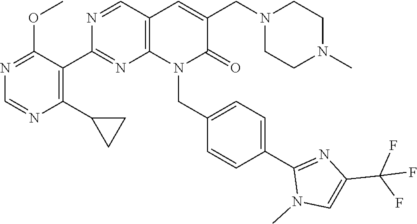 [SMILES: COc1ncnc(C2CC2)c1-c1ncc2cc(CN3CCN(C)CC3)c(=O)n(Cc3ccc(-c4nc(C(F)(F)F)cn4C)cc3)c2n1] | I-55 |
| 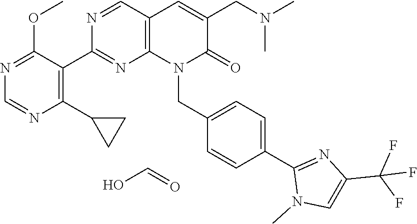 | I-56 |
| 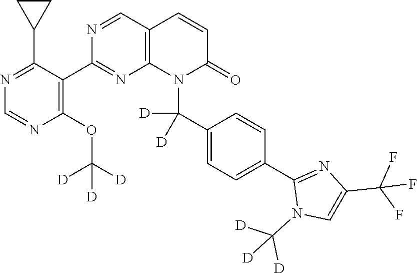 [SMILES: [2H]C([2H])([2H])Oc1ncnc(C2CC2)c1-c1ncc2ccc(=O)n(C([2H])([2H])c3ccc(-c4nc(C(F)(F)F)cn4C([2H])([2H])[2H])cc3)c2n1] | I-57 |
| 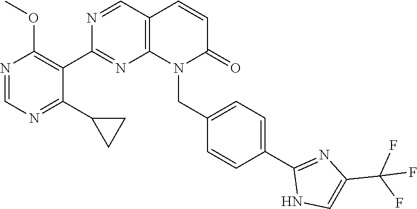 | I-58 |
| 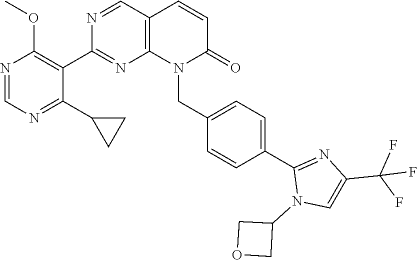 [SMILES: COc1ncnc(C2CC2)c1-c1ncc2ccc(=O)n(Cc3ccc(-c4nc(C(F)(F)F)cn4C4COC4)cc3)c2n1] | I-59 |
| 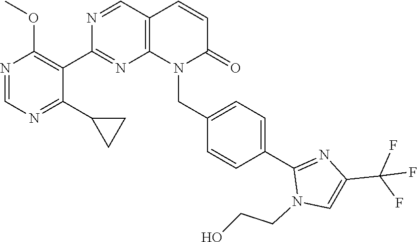 [SMILES: COc1ncnc(C2CC2)c1-c1ncc2ccc(=O)n(Cc3ccc(-c4nc(C(F)(F)F)cn4CCO)cc3)c2n1] | I-60 |
| 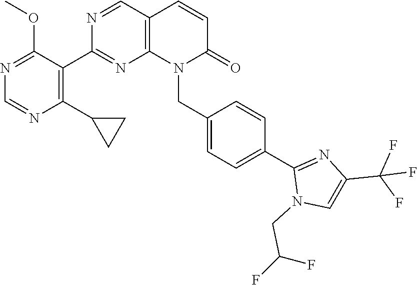 [SMILES: COc1ncnc(C2CC2)c1-c1ncc2ccc(=O)n(Cc3ccc(-c4nc(C(F)(F)F)cn4CC(F)F)cc3)c2n1] | I-61 |
| 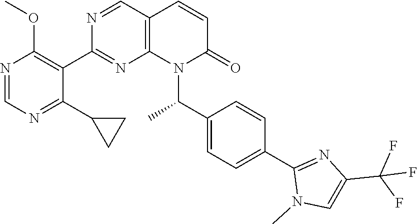 [SMILES: COc1ncnc(C2CC2)c1-c1ncc2ccc(=O)n([C@@H](C)c3ccc(-c4nc(C(F)(F)F)cn4C)cc3)c2n1] | I-62 |
| 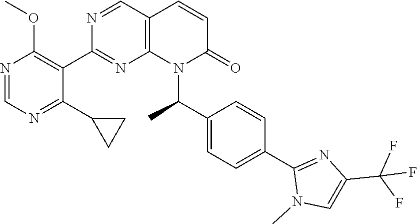 [SMILES: COc1ncnc(C2CC2)c1-c1ncc2ccc(=O)n([C@H](C)c3ccc(-c4nc(C(F)(F)F)cn4C)cc3)c2n1] | I-63 |
| 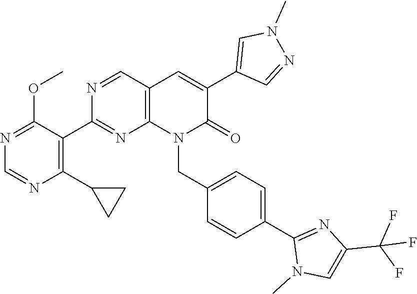 [SMILES: COc1ncnc(C2CC2)c1-c1ncc2cc(-c3cnn(C)c3)c(=O)n(Cc3ccc(-c4nc(C(F)(F)F)cn4C)cc3)c2n1] | I-64 |
| 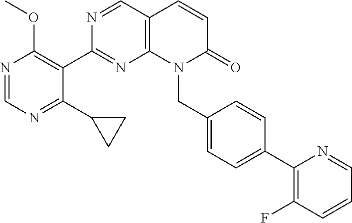 [SMILES: COc1ncnc(C2CC2)c1-c1ncc2ccc(=O)n(Cc3ccc(-c4ncccc4F)cc3)c2n1] | I-65 |
| 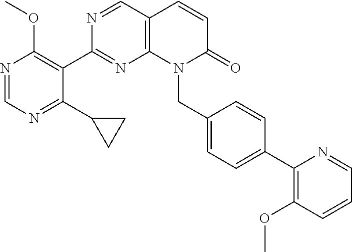 [SMILES: COc1cccnc1-c1ccc(Cn2c(=O)ccc3cnc(-c4c(OC)ncnc4C4CC4)nc32)cc1] | I-66 |
| 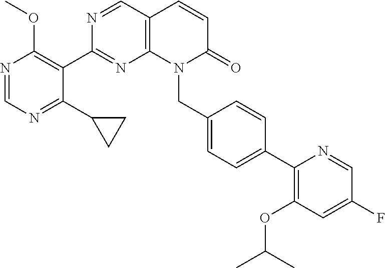 [SMILES: COc1ncnc(C2CC2)c1-c1ncc2ccc(=O)n(Cc3ccc(-c4ncc(F)cc4OC(C)C)cc3)c2n1] | I-67 |
| 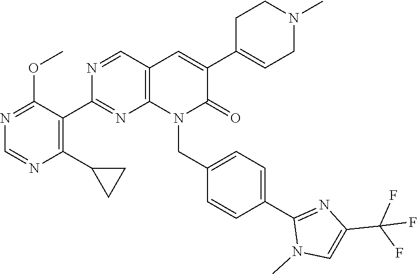 [SMILES: COc1ncnc(C2CC2)c1-c1ncc2cc(C3=CCN(C)CC3)c(=O)n(Cc3ccc(-c4nc(C(F)(F)F)cn4C)cc3)c2n1] | I-68 |
| 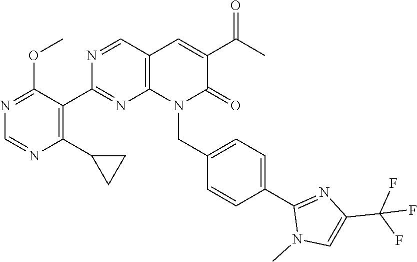 [SMILES: COc1ncnc(C2CC2)c1-c1ncc2cc(C(C)=O)c(=O)n(Cc3ccc(-c4nc(C(F)(F)F)cn4C)cc3)c2n1] | I-69 |
| 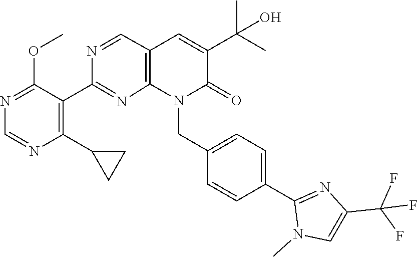 | I-70 |
| 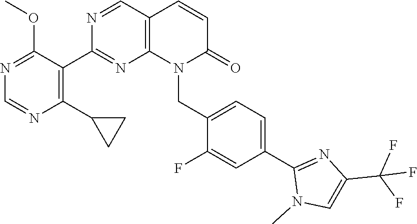 [SMILES: COc1ncnc(C2CC2)c1-c1ncc2ccc(=O)n(Cc3ccc(-c4nc(C(F)(F)F)cn4C)cc3F)c2n1] | I-71 |
| 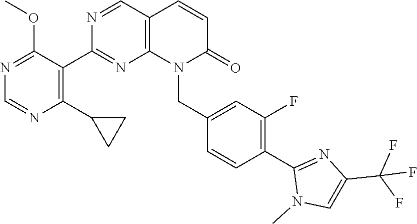 | I-72 |
| 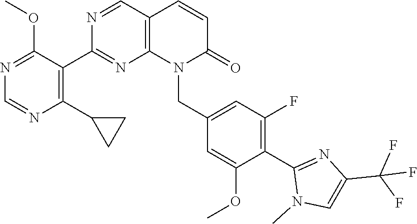 | I-73 |
| 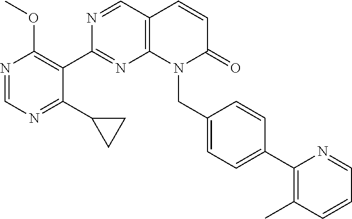 [SMILES: COc1ncnc(C2CC2)c1-c1ncc2ccc(=O)n(Cc3ccc(-c4ncccc4C)cc3)c2n1] | I-74 |
| 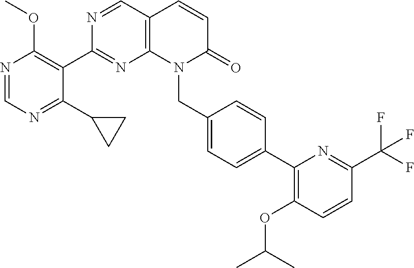 [SMILES: COc1ncnc(C2CC2)c1-c1ncc2ccc(=O)n(Cc3ccc(-c4nc(C(F)(F)F)ccc4OC(C)C)cc3)c2n1] | I-75 |
| 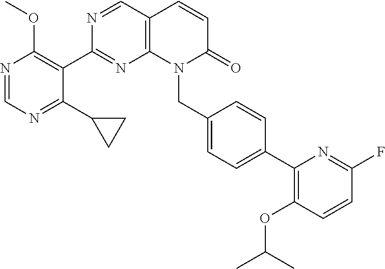 [SMILES: COc1ncnc(C2CC2)c1-c1ncc2ccc(=O)n(Cc3ccc(-c4nc(F)ccc4OC(C)C)cc3)c2n1] | I-76 |
| 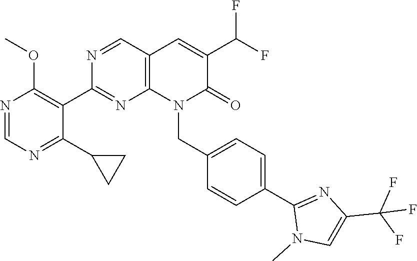 [SMILES: COc1ncnc(C2CC2)c1-c1ncc2cc(C(F)F)c(=O)n(Cc3ccc(-c4nc(C(F)(F)F)cn4C)cc3)c2n1] | I-77 |
| 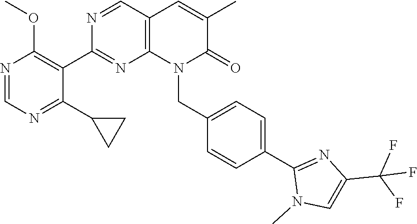 [SMILES: COc1ncnc(C2CC2)c1-c1ncc2cc(C)c(=O)n(Cc3ccc(-c4nc(C(F)(F)F)cn4C)cc3)c2n1] | I-78 |
| 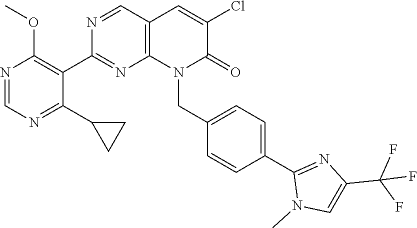 [SMILES: COc1ncnc(C2CC2)c1-c1ncc2cc(Cl)c(=O)n(Cc3ccc(-c4nc(C(F)(F)F)cn4C)cc3)c2n1] | I-79 |
| 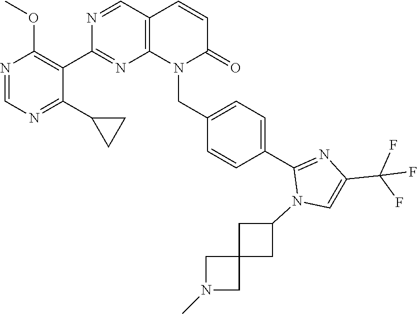 [SMILES: COc1ncnc(C2CC2)c1-c1ncc2ccc(=O)n(Cc3ccc(-c4nc(C(F)(F)F)cn4C4CC5(C4)CN(C)C5)cc3)c2n1] | I-80 |
| 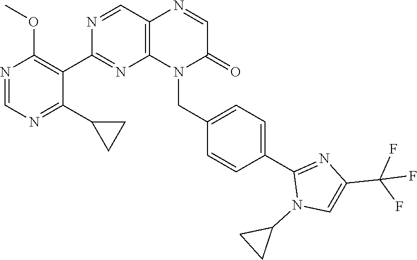 [SMILES: COc1ncnc(C2CC2)c1-c1ncc2ncc(=O)n(Cc3ccc(-c4nc(C(F)(F)F)cn4C4CC4)cc3)c2n1] | I-81 |
| 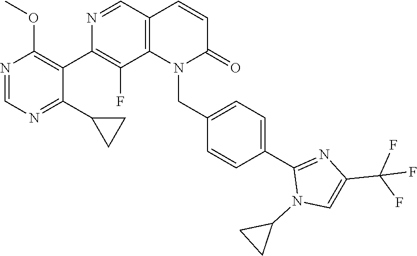 [SMILES: COc1ncnc(C2CC2)c1-c1ncc2ccc(=O)n(Cc3ccc(-c4nc(C(F)(F)F)cn4C4CC4)cc3)c2c1F] | I-82 |
| 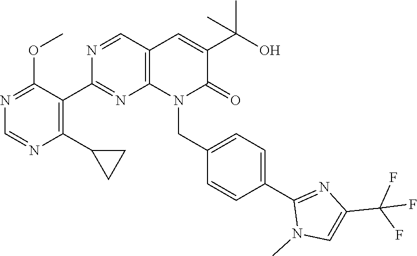 [SMILES: COc1ncnc(C2CC2)c1-c1ncc2cc(C(C)(C)O)c(=O)n(Cc3ccc(-c4nc(C(F)(F)F)cn4C)cc3)c2n1] | I-83 |
| 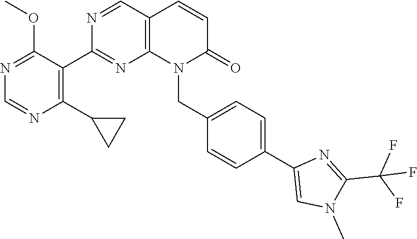 [SMILES: COc1ncnc(C2CC2)c1-c1ncc2ccc(=O)n(Cc3ccc(-c4cn(C)c(C(F)(F)F)n4)cc3)c2n1] | I-84 |
| 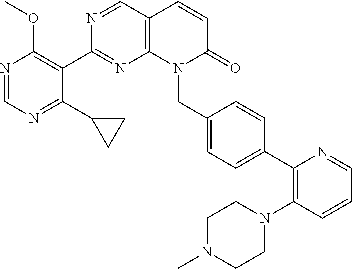 [SMILES: COc1ncnc(C2CC2)c1-c1ncc2ccc(=O)n(Cc3ccc(-c4ncccc4N4CCN(C)CC4)cc3)c2n1] | I-85 |
| 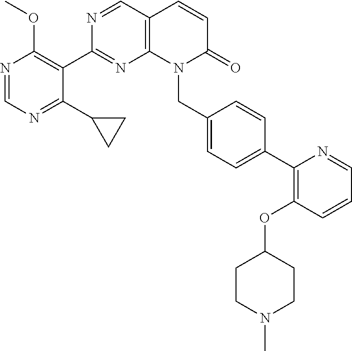 [SMILES: COc1ncnc(C2CC2)c1-c1ncc2ccc(=O)n(Cc3ccc(-c4ncccc4OC4CCN(C)CC4)cc3)c2n1] | I-86 |
| 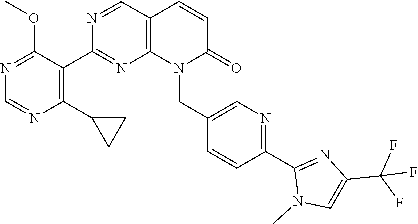 | I-87 |
| 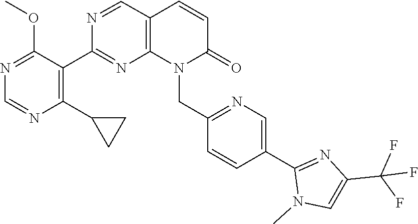 | I-88 |
| 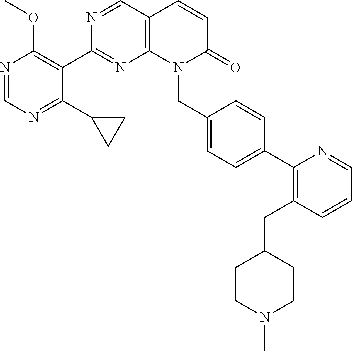 [SMILES: COc1ncnc(C2CC2)c1-c1ncc2ccc(=O)n(Cc3ccc(-c4ncccc4CC4CCN(C)CC4)cc3)c2n1] | I-89 |
| 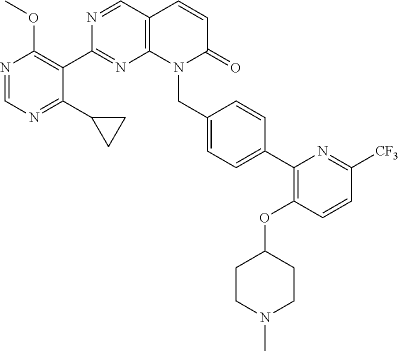 [SMILES: COc1ncnc(C2CC2)c1-c1ncc2ccc(=O)n(Cc3ccc(-c4nc(C(F)(F)F)ccc4OC4CCN(C)CC4)cc3)c2n1] | I-90 |
| 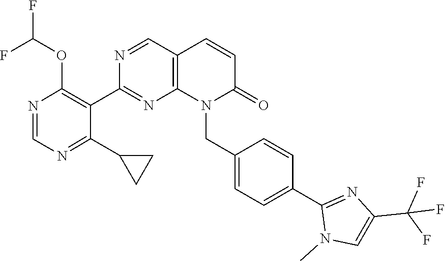 | I-91 |
| 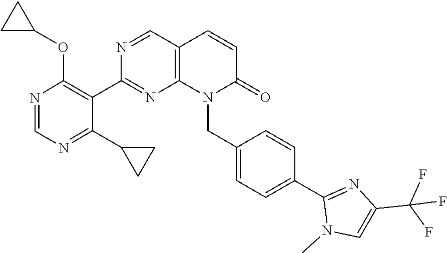 | I-92 |
| 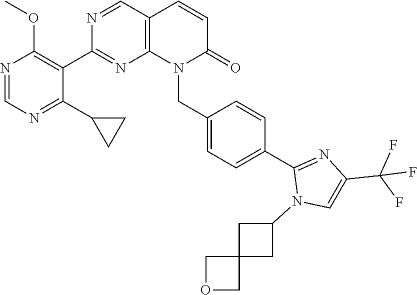 [SMILES: COc1ncnc(C2CC2)c1-c1ncc2ccc(=O)n(Cc3ccc(-c4nc(C(F)(F)F)cn4C4CC5(COC5)C4)cc3)c2n1] | I-93 |
| 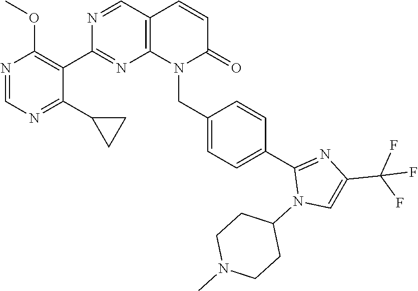 [SMILES: COc1ncnc(C2CC2)c1-c1ncc2ccc(=O)n(Cc3ccc(-c4nc(C(F)(F)F)cn4C4CCN(C)CC4)cc3)c2n1] | I-94 |
| 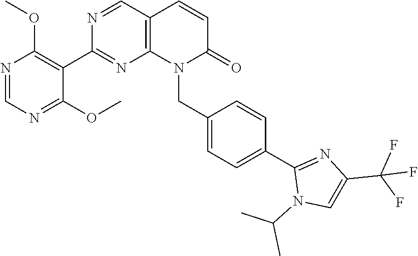 [SMILES: COc1ncnc(OC)c1-c1ncc2ccc(=O)n(Cc3ccc(-c4nc(C(F)(F)F)cn4C(C)C)cc3)c2n1] | I-95 |
| 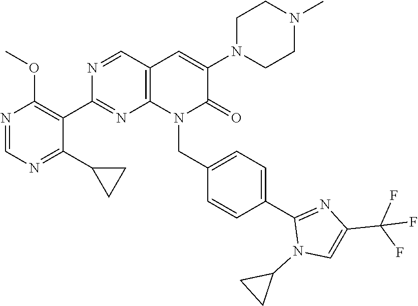 [SMILES: COc1ncnc(C2CC2)c1-c1ncc2cc(N3CCN(C)CC3)c(=O)n(Cc3ccc(-c4nc(C(F)(F)F)cn4C4CC4)cc3)c2n1] | I-96 |
| 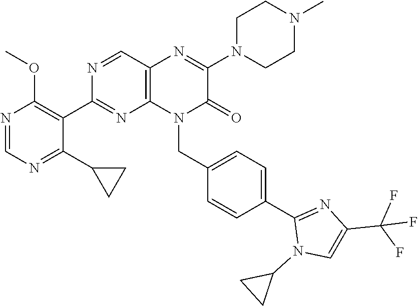 [SMILES: COc1ncnc(C2CC2)c1-c1ncc2nc(N3CCN(C)CC3)c(=O)n(Cc3ccc(-c4nc(C(F)(F)F)cn4C4CC4)cc3)c2n1] | I-97 |
| 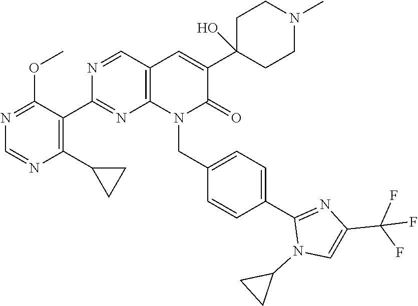 [SMILES: COc1ncnc(C2CC2)c1-c1ncc2cc(C3(O)CCN(C)CC3)c(=O)n(Cc3ccc(-c4nc(C(F)(F)F)cn4C4CC4)cc3)c2n1] | I-98 |
| 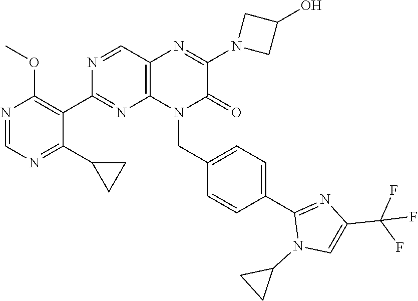 [SMILES: COc1ncnc(C2CC2)c1-c1ncc2nc(N3CC(O)C3)c(=O)n(Cc3ccc(-c4nc(C(F)(F)F)cn4C4CC4)cc3)c2n1] | I-99 |
| 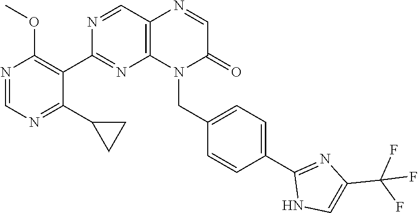 [SMILES: COc1ncnc(C2CC2)c1-c1ncc2ncc(=O)n(Cc3ccc(-c4nc(C(F)(F)F)c[nH]4)cc3)c2n1] | I-100 |
| 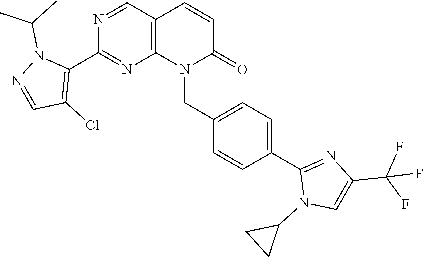 [SMILES: CC(C)n1ncc(Cl)c1-c1ncc2ccc(=O)n(Cc3ccc(-c4nc(C(F)(F)F)cn4C4CC4)cc3)c2n1] | I-101 |
| 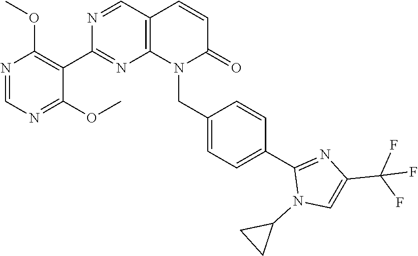 [SMILES: COc1ncnc(OC)c1-c1ncc2ccc(=O)n(Cc3ccc(-c4nc(C(F)(F)F)cn4C4CC4)cc3)c2n1] | I-102 |
| 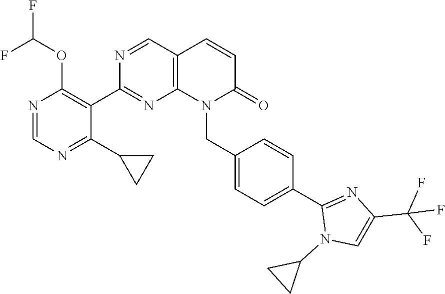 [SMILES: O=c1ccc2cnc(-c3c(OC(F)F)ncnc3C3CC3)nc2n1Cc1ccc(-c2nc(C(F)(F)F)cn2C2CC2)cc1] | I-103 |
| 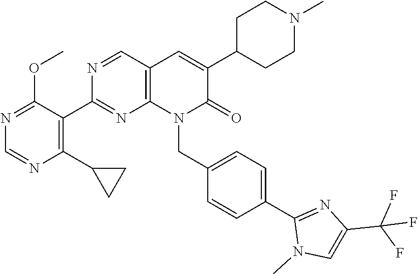 [SMILES: COc1ncnc(C2CC2)c1-c1ncc2cc(C3CCN(C)CC3)c(=O)n(Cc3ccc(-c4nc(C(F)(F)F)cn4C)cc3)c2n1] | I-104 |
| 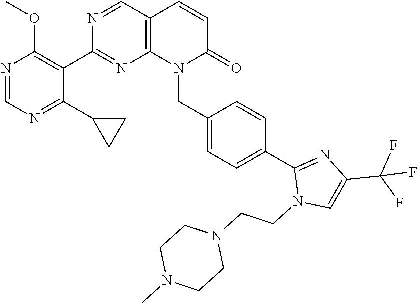 [SMILES: COc1ncnc(C2CC2)c1-c1ncc2ccc(=O)n(Cc3ccc(-c4nc(C(F)(F)F)cn4CCN4CCN(C)CC4)cc3)c2n1] | I-105 |
| 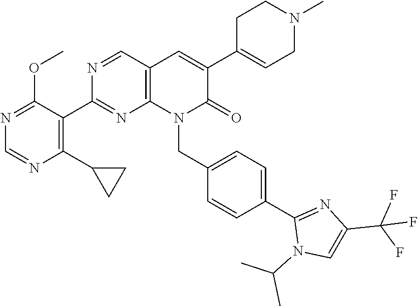 [SMILES: COc1ncnc(C2CC2)c1-c1ncc2cc(C3=CCN(C)CC3)c(=O)n(Cc3ccc(-c4nc(C(F)(F)F)cn4C(C)C)cc3)c2n1] | I-106 |
| 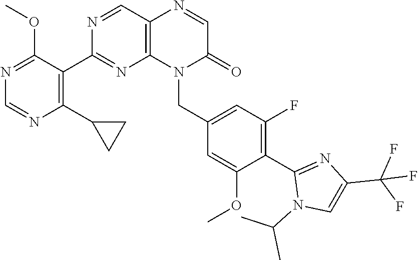 [SMILES: COc1cc(Cn2c(=O)cnc3cnc(-c4c(OC)ncnc4C4CC4)nc32)cc(F)c1-c1nc(C(F)(F)F)cn1C(C)C] | I-107 |
| 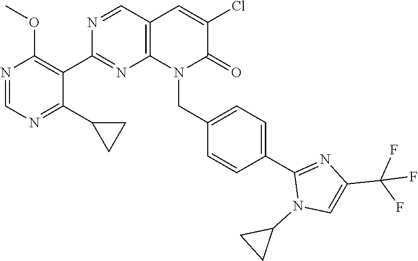 [SMILES: COc1ncnc(C2CC2)c1-c1ncc2cc(Cl)c(=O)n(Cc3ccc(-c4nc(C(F)(F)F)cn4C4CC4)cc3)c2n1] | I-108 |
| 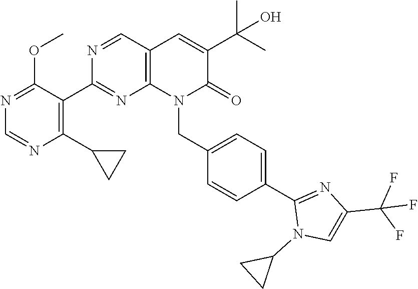 [SMILES: COc1ncnc(C2CC2)c1-c1ncc2cc(C(C)(C)O)c(=O)n(Cc3ccc(-c4nc(C(F)(F)F)cn4C4CC4)cc3)c2n1] | I-109 |
| 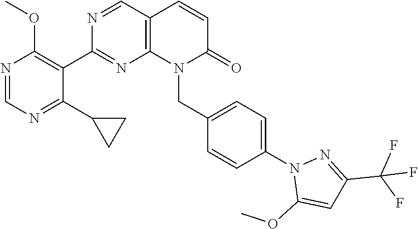 [SMILES: COc1ncnc(C2CC2)c1-c1ncc2ccc(=O)n(Cc3ccc(-n4nc(C(F)(F)F)cc4OC)cc3)c2n1] | I-110 |
| 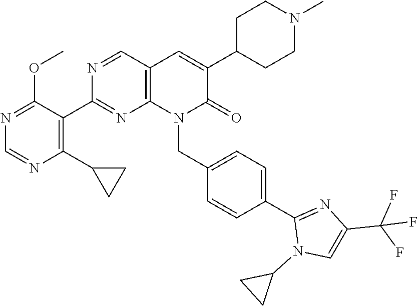 [SMILES: COc1ncnc(C2CC2)c1-c1ncc2cc(C3CCN(C)CC3)c(=O)n(Cc3ccc(-c4nc(C(F)(F)F)cn4C4CC4)cc3)c2n1] | I-111 |
| 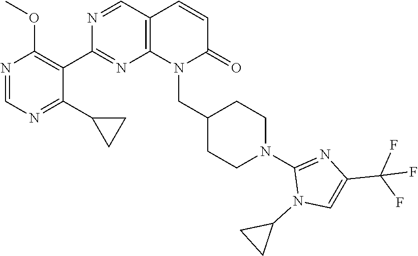 [SMILES: COc1ncnc(C2CC2)c1-c1ncc2ccc(=O)n(CC3CCN(c4nc(C(F)(F)F)cn4C4CC4)CC3)c2n1] | I-112 |
| 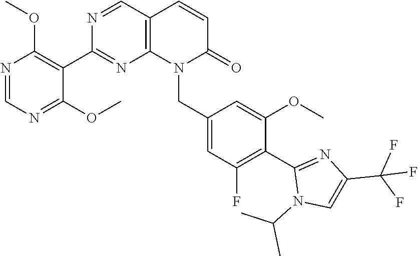 [SMILES: COc1cc(Cn2c(=O)ccc3cnc(-c4c(OC)ncnc4OC)nc32)cc(F)c1-c1nc(C(F)(F)F)cn1C(C)C] | I-113 |
| 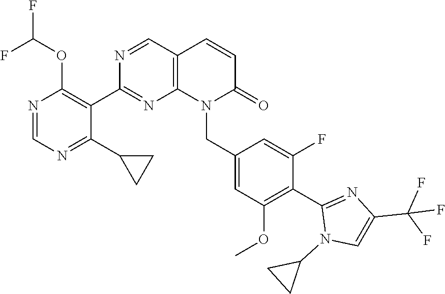 [SMILES: COc1cc(Cn2c(=O)ccc3cnc(-c4c(OC(F)F)ncnc4C4CC4)nc32)cc(F)c1-c1nc(C(F)(F)F)cn1C1CC1] | I-114 |
| 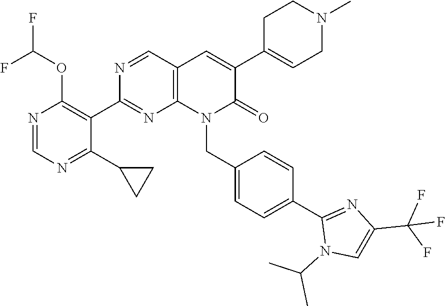 [SMILES: CC(C)n1cc(C(F)(F)F)nc1-c1ccc(Cn2c(=O)c(C3=CCN(C)CC3)cc3cnc(-c4c(OC(F)F)ncnc4C4CC4)nc32)cc1] | I-115 |
| 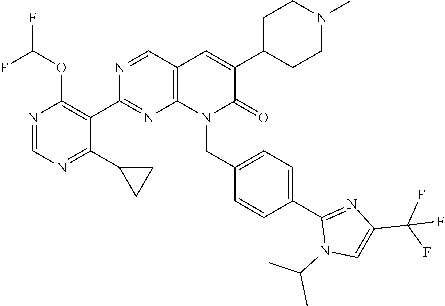 [SMILES: CC(C)n1cc(C(F)(F)F)nc1-c1ccc(Cn2c(=O)c(C3CCN(C)CC3)cc3cnc(-c4c(OC(F)F)ncnc4C4CC4)nc32)cc1] | I-116 |
| 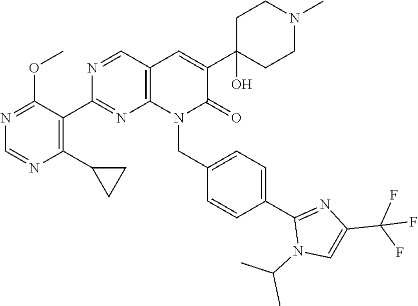 [SMILES: COc1ncnc(C2CC2)c1-c1ncc2cc(C3(O)CCN(C)CC3)c(=O)n(Cc3ccc(-c4nc(C(F)(F)F)cn4C(C)C)cc3)c2n1] | I-117 |
| 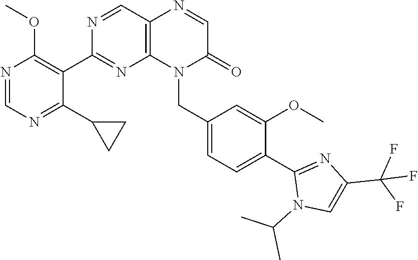 [SMILES: COc1cc(Cn2c(=O)cnc3cnc(-c4c(OC)ncnc4C4CC4)nc32)ccc1-c1nc(C(F)(F)F)cn1C(C)C] | I-118 |
| 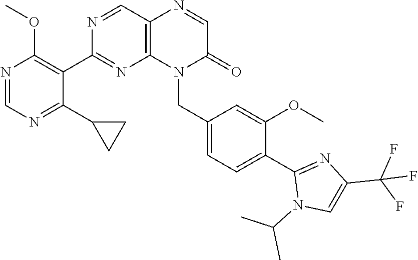 [SMILES: COc1cc(Cn2c(=O)cnc3cnc(-c4c(OC)ncnc4C4CC4)nc32)ccc1-c1nc(C(F)(F)F)cn1C(C)C] | I-119 |
| 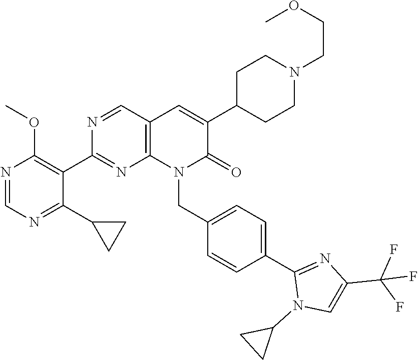 [SMILES: COCCN1CCC(c2cc3cnc(-c4c(OC)ncnc4C4CC4)nc3n(Cc3ccc(-c4nc(C(F)(F)F)cn4C4CC4)cc3)c2=O)CC1] | I-120 |
| 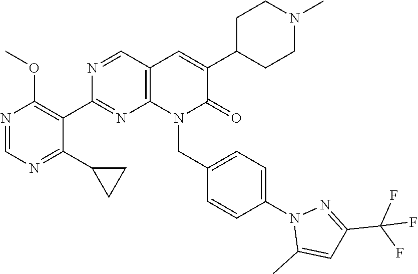 [SMILES: COc1ncnc(C2CC2)c1-c1ncc2cc(C3CCN(C)CC3)c(=O)n(Cc3ccc(-n4nc(C(F)(F)F)cc4C)cc3)c2n1] | I-121 |
| 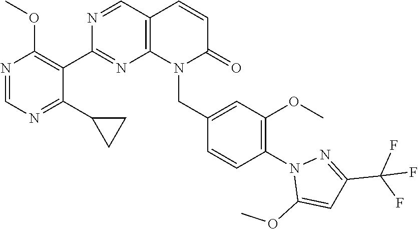 [SMILES: COc1cc(Cn2c(=O)ccc3cnc(-c4c(OC)ncnc4C4CC4)nc32)ccc1-n1nc(C(F)(F)F)cc1OC] | I-122 |
| 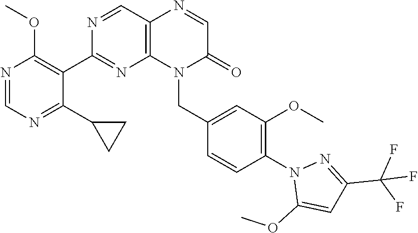 [SMILES: COc1cc(Cn2c(=O)cnc3cnc(-c4c(OC)ncnc4C4CC4)nc32)ccc1-n1nc(C(F)(F)F)cc1OC] | I-123 |
| 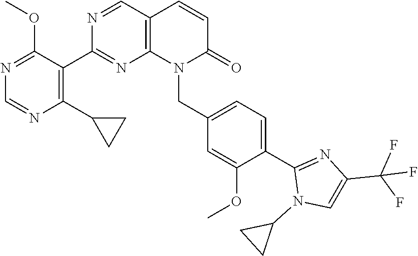 [SMILES: COc1cc(Cn2c(=O)ccc3cnc(-c4c(OC)ncnc4C4CC4)nc32)ccc1-c1nc(C(F)(F)F)cn1C1CC1] | I-124 |
| 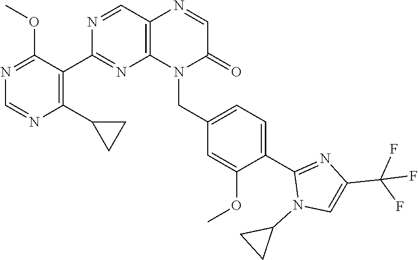 [SMILES: COc1cc(Cn2c(=O)cnc3cnc(-c4c(OC)ncnc4C4CC4)nc32)ccc1-c1nc(C(F)(F)F)cn1C1CC1] | I-125 |
| 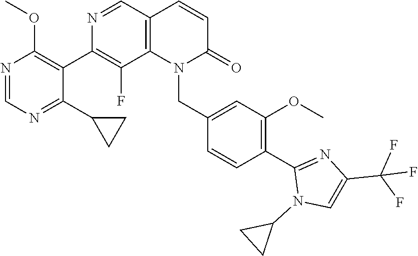 [SMILES: COc1cc(Cn2c(=O)ccc3cnc(-c4c(OC)ncnc4C4CC4)c(F)c32)ccc1-c1nc(C(F)(F)F)cn1C1CC1] | I-126 |
| 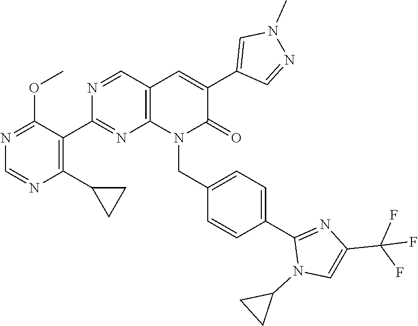 [SMILES: COc1ncnc(C2CC2)c1-c1ncc2cc(-c3cnn(C)c3)c(=O)n(Cc3ccc(-c4nc(C(F)(F)F)cn4C4CC4)cc3)c2n1] | I-127 |
| 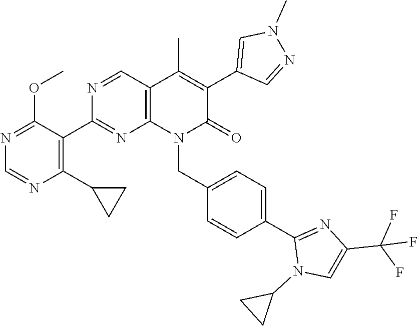 [SMILES: COc1ncnc(C2CC2)c1-c1ncc2c(C)c(-c3cnn(C)c3)c(=O)n(Cc3ccc(-c4nc(C(F)(F)F)cn4C4CC4)cc3)c2n1] | I-128 |
| 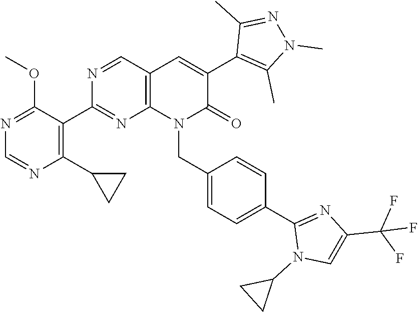 [SMILES: COc1ncnc(C2CC2)c1-c1ncc2cc(-c3c(C)nn(C)c3C)c(=O)n(Cc3ccc(-c4nc(C(F)(F)F)cn4C4CC4)cc3)c2n1] | I-129 |
| 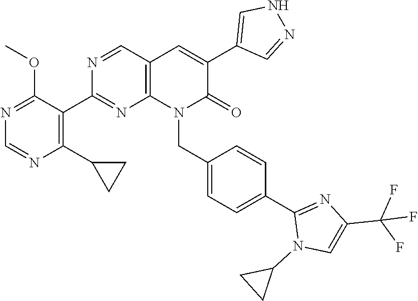 [SMILES: COc1ncnc(C2CC2)c1-c1ncc2cc(-c3cn[nH]c3)c(=O)n(Cc3ccc(-c4nc(C(F)(F)F)cn4C4CC4)cc3)c2n1] | I-130 |
| 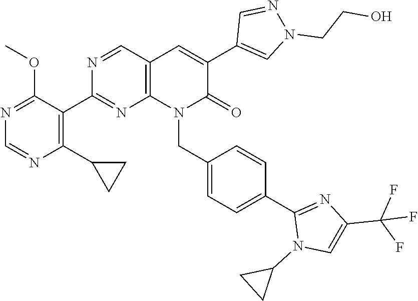 [SMILES: COc1ncnc(C2CC2)c1-c1ncc2cc(-c3cnn(CCO)c3)c(=O)n(Cc3ccc(-c4nc(C(F)(F)F)cn4C4CC4)cc3)c2n1] | I-131 |
| 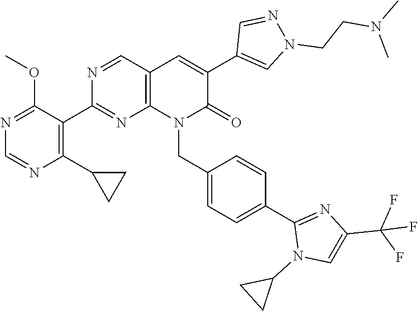 [SMILES: COc1ncnc(C2CC2)c1-c1ncc2cc(-c3cnn(CCN(C)C)c3)c(=O)n(Cc3ccc(-c4nc(C(F)(F)F)cn4C4CC4)cc3)c2n1] | I-132 |
| 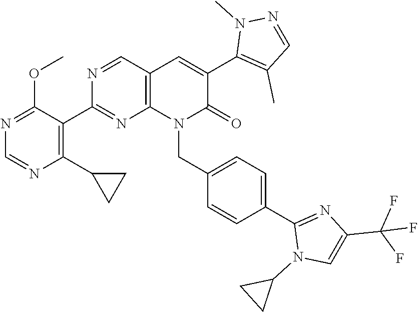 [SMILES: COc1ncnc(C2CC2)c1-c1ncc2cc(-c3c(C)cnn3C)c(=O)n(Cc3ccc(-c4nc(C(F)(F)F)cn4C4CC4)cc3)c2n1] | I-133 |
| 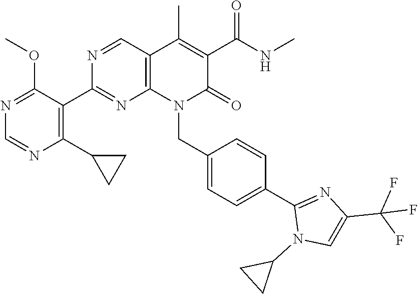 [SMILES: CNC(=O)c1c(C)c2cnc(-c3c(OC)ncnc3C3CC3)nc2n(Cc2ccc(-c3nc(C(F)(F)F)cn3C3CC3)cc2)c1=O] | I-134 |
| 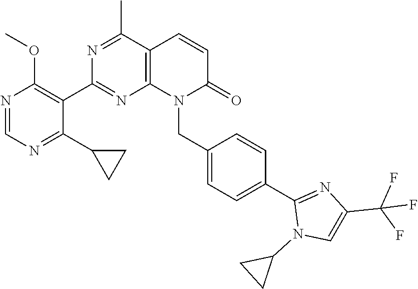 [SMILES: COc1ncnc(C2CC2)c1-c1nc(C)c2ccc(=O)n(Cc3ccc(-c4nc(C(F)(F)F)cn4C4CC4)cc3)c2n1] | I-135 |
| 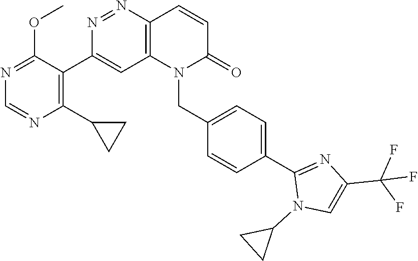 [SMILES: COc1ncnc(C2CC2)c1-c1cc2c(ccc(=O)n2Cc2ccc(-c3nc(C(F)(F)F)cn3C3CC3)cc2)nn1] | I-136 |
| 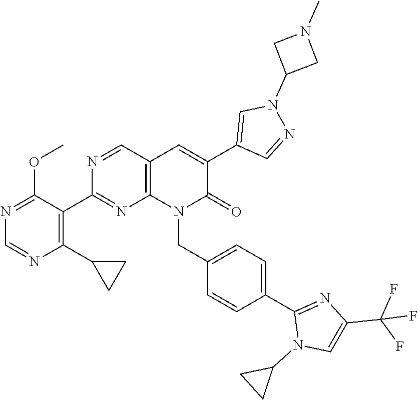 [SMILES: COc1ncnc(C2CC2)c1-c1ncc2cc(-c3cnn(C4CN(C)C4)c3)c(=O)n(Cc3ccc(-c4nc(C(F)(F)F)cn4C4CC4)cc3)c2n1] | I-137 |
| 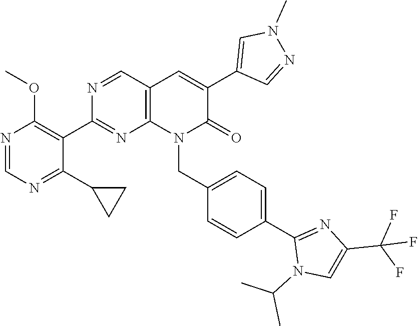 [SMILES: COc1ncnc(C2CC2)c1-c1ncc2cc(-c3cnn(C)c3)c(=O)n(Cc3ccc(-c4nc(C(F)(F)F)cn4C(C)C)cc3)c2n1] | I-138 |
| 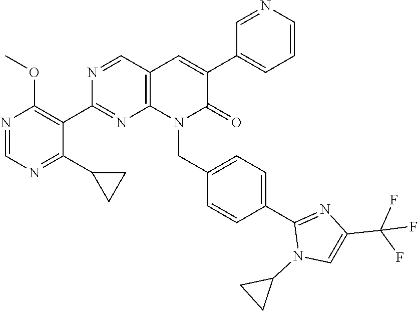 [SMILES: COc1ncnc(C2CC2)c1-c1ncc2cc(-c3cccnc3)c(=O)n(Cc3ccc(-c4nc(C(F)(F)F)cn4C4CC4)cc3)c2n1] | I-139 |
| 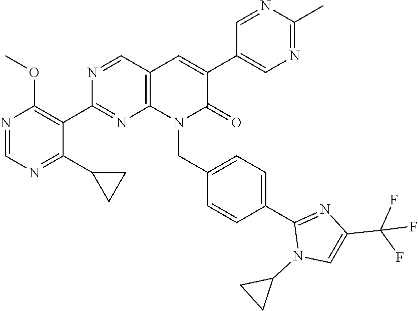 [SMILES: COc1ncnc(C2CC2)c1-c1ncc2cc(-c3cnc(C)nc3)c(=O)n(Cc3ccc(-c4nc(C(F)(F)F)cn4C4CC4)cc3)c2n1] | I-140 |
| 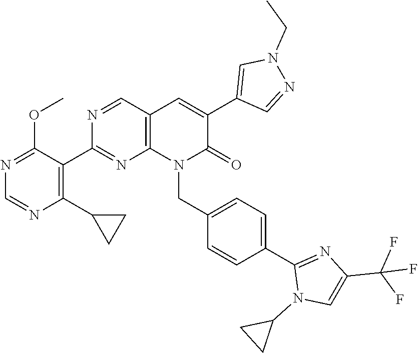 [SMILES: CCn1cc(-c2cc3cnc(-c4c(OC)ncnc4C4CC4)nc3n(Cc3ccc(-c4nc(C(F)(F)F)cn4C4CC4)cc3)c2=O)cn1] | I-141 |
| 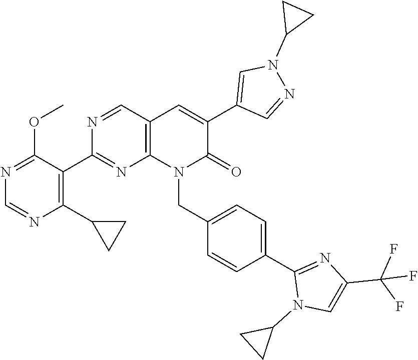 [SMILES: COc1ncnc(C2CC2)c1-c1ncc2cc(-c3cnn(C4CC4)c3)c(=O)n(Cc3ccc(-c4nc(C(F)(F)F)cn4C4CC4)cc3)c2n1] | I-142 |
| 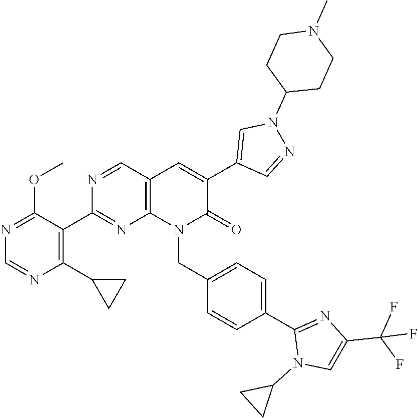 [SMILES: COc1ncnc(C2CC2)c1-c1ncc2cc(-c3cnn(C4CCN(C)CC4)c3)c(=O)n(Cc3ccc(-c4nc(C(F)(F)F)cn4C4CC4)cc3)c2n1] | I-143 |
| 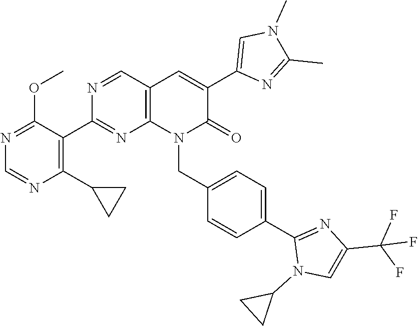 [SMILES: COc1ncnc(C2CC2)c1-c1ncc2cc(-c3cn(C)c(C)n3)c(=O)n(Cc3ccc(-c4nc(C(F)(F)F)cn4C4CC4)cc3)c2n1] | I-144 |
| 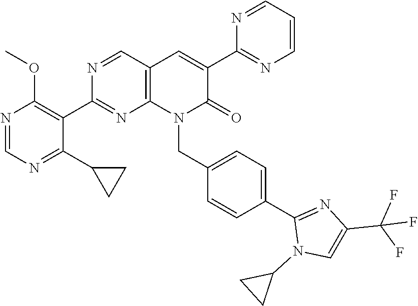 [SMILES: COc1ncnc(C2CC2)c1-c1ncc2cc(-c3ncccn3)c(=O)n(Cc3ccc(-c4nc(C(F)(F)F)cn4C4CC4)cc3)c2n1] | I-145 |
| 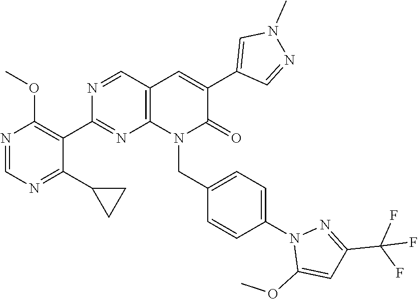 [SMILES: COc1ncnc(C2CC2)c1-c1ncc2cc(-c3cnn(C)c3)c(=O)n(Cc3ccc(-n4nc(C(F)(F)F)cc4OC)cc3)c2n1] | I-146 |
| 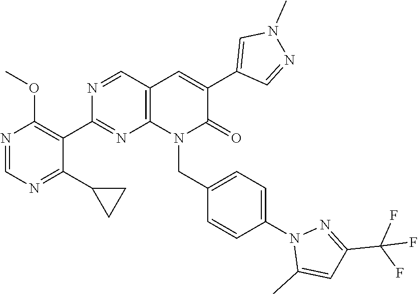 [SMILES: COc1ncnc(C2CC2)c1-c1ncc2cc(-c3cnn(C)c3)c(=O)n(Cc3ccc(-n4nc(C(F)(F)F)cc4C)cc3)c2n1] | I-147 |
| 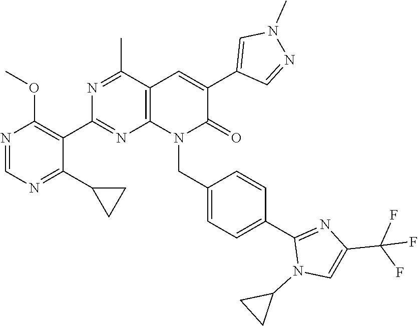 [SMILES: COc1ncnc(C2CC2)c1-c1nc(C)c2cc(-c3cnn(C)c3)c(=O)n(Cc3ccc(-c4nc(C(F)(F)F)cn4C4CC4)cc3)c2n1] | I-148 |
| 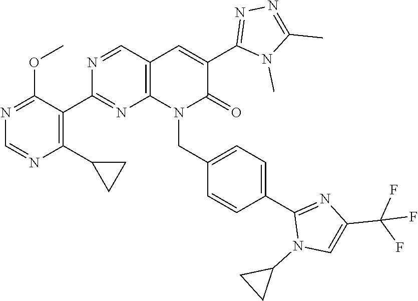 [SMILES: COc1ncnc(C2CC2)c1-c1ncc2cc(-c3nnc(C)n3C)c(=O)n(Cc3ccc(-c4nc(C(F)(F)F)cn4C4CC4)cc3)c2n1] | I-149 |
| 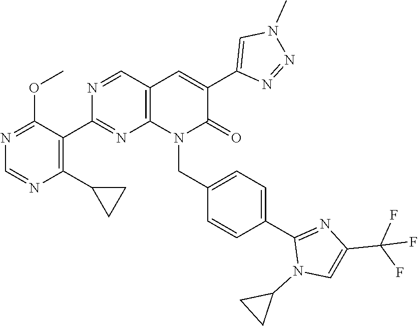 [SMILES: COc1ncnc(C2CC2)c1-c1ncc2cc(-c3cn(C)nn3)c(=O)n(Cc3ccc(-c4nc(C(F)(F)F)cn4C4CC4)cc3)c2n1] | I-150 |
| 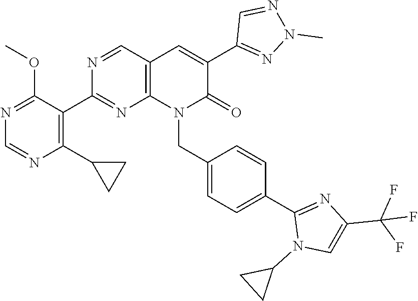 [SMILES: COc1ncnc(C2CC2)c1-c1ncc2cc(-c3cnn(C)n3)c(=O)n(Cc3ccc(-c4nc(C(F)(F)F)cn4C4CC4)cc3)c2n1] | I-151 |
| 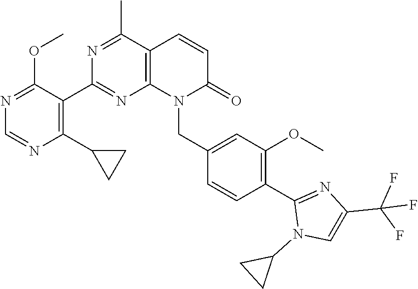 [SMILES: COc1cc(Cn2c(=O)ccc3c(C)nc(-c4c(OC)ncnc4C4CC4)nc32)ccc1-c1nc(C(F)(F)F)cn1C1CC1] | I-152 |
| 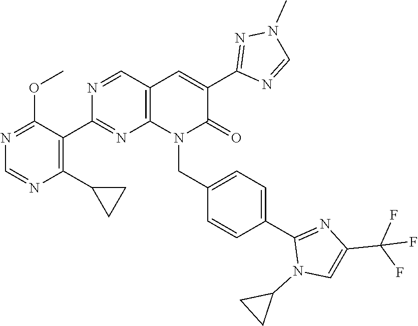 [SMILES: COc1ncnc(C2CC2)c1-c1ncc2cc(-c3ncn(C)n3)c(=O)n(Cc3ccc(-c4nc(C(F)(F)F)cn4C4CC4)cc3)c2n1] | I-153 |
| 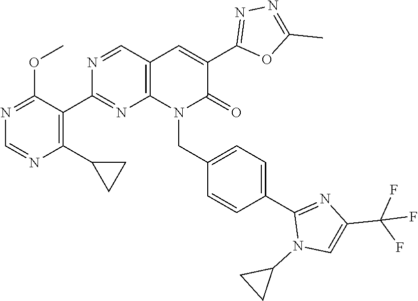 [SMILES: COc1ncnc(C2CC2)c1-c1ncc2cc(-c3nnc(C)o3)c(=O)n(Cc3ccc(-c4nc(C(F)(F)F)cn4C4CC4)cc3)c2n1] | I-154 |
| 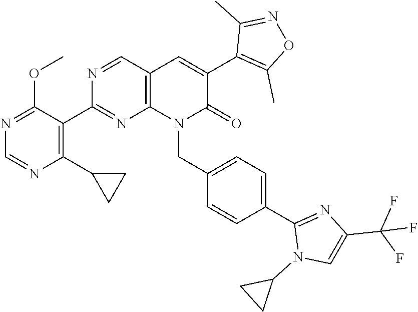 [SMILES: COc1ncnc(C2CC2)c1-c1ncc2cc(-c3c(C)noc3C)c(=O)n(Cc3ccc(-c4nc(C(F)(F)F)cn4C4CC4)cc3)c2n1] | I-155 |
| 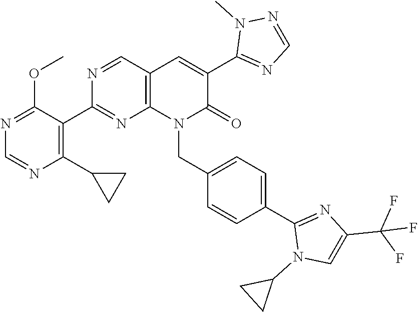 [SMILES: COc1ncnc(C2CC2)c1-c1ncc2cc(-c3ncnn3C)c(=O)n(Cc3ccc(-c4nc(C(F)(F)F)cn4C4CC4)cc3)c2n1] | I-156 |
| 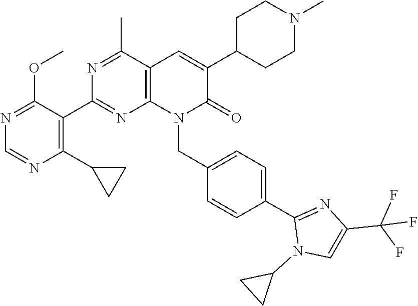 [SMILES: COc1ncnc(C2CC2)c1-c1nc(C)c2cc(C3CCN(C)CC3)c(=O)n(Cc3ccc(-c4nc(C(F)(F)F)cn4C4CC4)cc3)c2n1] | I-157 |
| 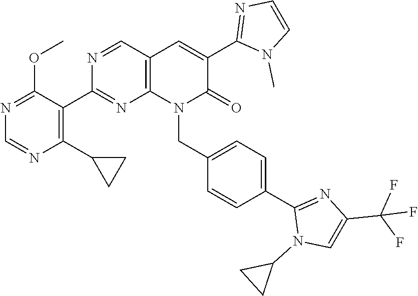 [SMILES: COc1ncnc(C2CC2)c1-c1ncc2cc(-c3nccn3C)c(=O)n(Cc3ccc(-c4nc(C(F)(F)F)cn4C4CC4)cc3)c2n1] | I-158 |
| 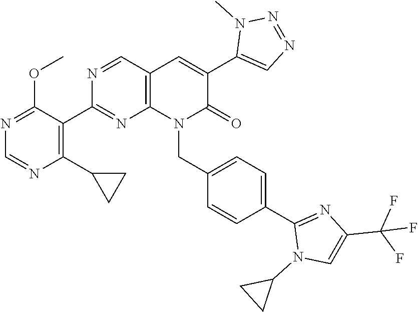 [SMILES: COc1ncnc(C2CC2)c1-c1ncc2cc(-c3cnnn3C)c(=O)n(Cc3ccc(-c4nc(C(F)(F)F)cn4C4CC4)cc3)c2n1] | I-159 |
| 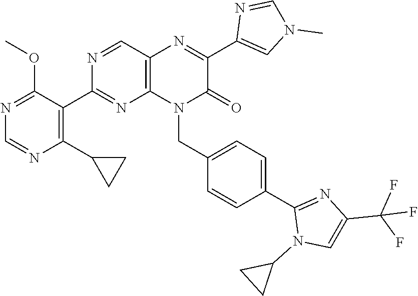 [SMILES: COc1ncnc(C2CC2)c1-c1ncc2nc(-c3cn(C)cn3)c(=O)n(Cc3ccc(-c4nc(C(F)(F)F)cn4C4CC4)cc3)c2n1] | I-160 |
| 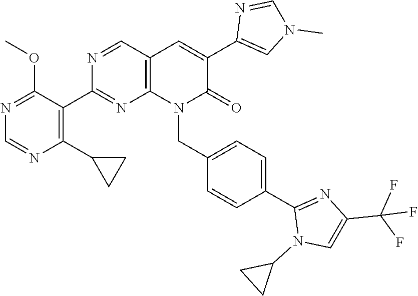 [SMILES: COc1ncnc(C2CC2)c1-c1ncc2cc(-c3cn(C)cn3)c(=O)n(Cc3ccc(-c4nc(C(F)(F)F)cn4C4CC4)cc3)c2n1] | I-161 |
| 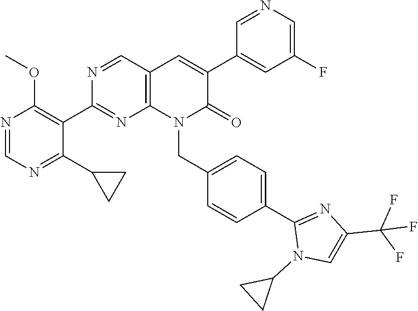 [SMILES: COc1ncnc(C2CC2)c1-c1ncc2cc(-c3cncc(F)c3)c(=O)n(Cc3ccc(-c4nc(C(F)(F)F)cn4C4CC4)cc3)c2n1] | I-162 |
| 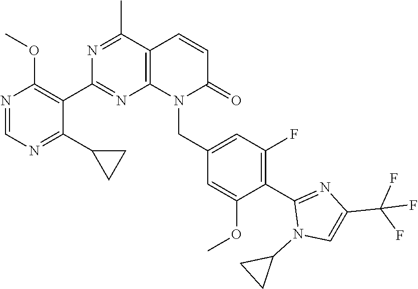 [SMILES: COc1cc(Cn2c(=O)ccc3c(C)nc(-c4c(OC)ncnc4C4CC4)nc32)cc(F)c1-c1nc(C(F)(F)F)cn1C1CC1] | I-163 |
| 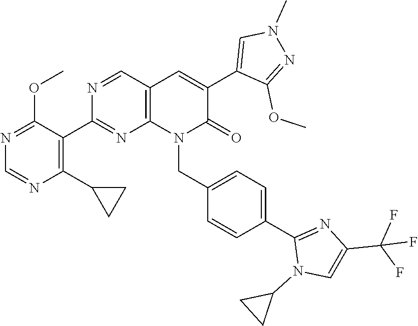 [SMILES: COc1nn(C)cc1-c1cc2cnc(-c3c(OC)ncnc3C3CC3)nc2n(Cc2ccc(-c3nc(C(F)(F)F)cn3C3CC3)cc2)c1=O] | I-164 |
| 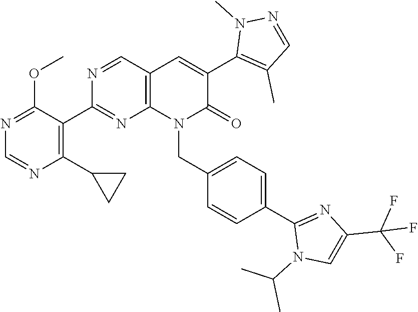 [SMILES: COc1ncnc(C2CC2)c1-c1ncc2cc(-c3c(C)cnn3C)c(=O)n(Cc3ccc(-c4nc(C(F)(F)F)cn4C(C)C)cc3)c2n1] | I-165 |
| 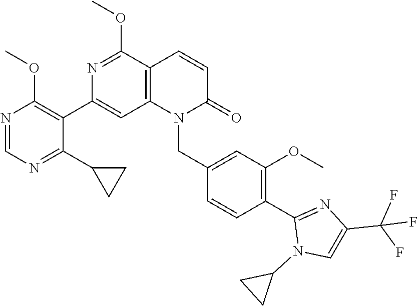 [SMILES: COc1cc(Cn2c(=O)ccc3c(OC)nc(-c4c(OC)ncnc4C4CC4)cc32)ccc1-c1nc(C(F)(F)F)cn1C1CC1] | I-166 |
| 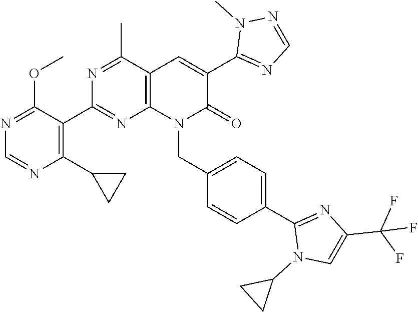 [SMILES: COc1ncnc(C2CC2)c1-c1nc(C)c2cc(-c3ncnn3C)c(=O)n(Cc3ccc(-c4nc(C(F)(F)F)cn4C4CC4)cc3)c2n1] | I-167 |
| 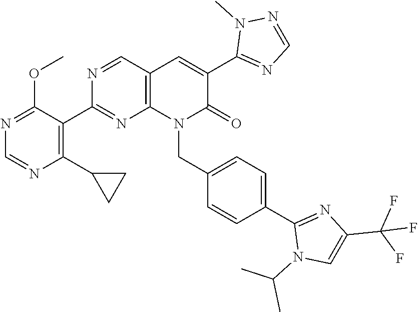 [SMILES: COc1ncnc(C2CC2)c1-c1ncc2cc(-c3ncnn3C)c(=O)n(Cc3ccc(-c4nc(C(F)(F)F)cn4C(C)C)cc3)c2n1] | I-168 |

The compounds of this invention can be prepared or isolated in general by synthetic and/or semi-synthetic methods known to those skilled in the art for analogous compounds and by methods described in detail in the Examples, herein. In some embodiments, the present invention provides an intermediate compound described in the Examples, or a salt thereof.

### 4. Uses, Formulation and Administration:

### Pharmaceutically Acceptable Compositions

According to another embodiment, the invention provides a pharmaceutical composition comprising a compound of this invention or a pharmaceutically acceptable derivative thereof and a pharmaceutically acceptable carrier, adjuvant, or vehicle. The amount of compound in compositions of this invention is such that is effective to measurably inhibit USP1, or a variant or mutant thereof, in a biological sample or in a patient. In certain embodiments, the amount of compound in compositions of this invention is such that is effective to measurably inhibit USP1, or a variant or mutant thereof, in a biological sample or in a patient. In certain embodiments, a composition of this invention is formulated for administration to a patient in need of such composition. In some embodiments, a composition of this invention is formulated for oral administration to a patient.

The terms “patient” or “subject” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of USP1, or a variant or mutant thereof.

Compositions of the present invention can be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention cancan be aqueous or oleaginous suspension. These suspensions cancan be formulated according to techniques known in the art using suitable dispersing or wetting agents and suspending agents. The sterile injectable preparation cancan also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium.

For this purpose, any bland fixed oil can be employed including synthetic mono-or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Pharmaceutically acceptable compositions of this invention can be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

Alternatively, pharmaceutically acceptable compositions of this invention can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For topical applications, provided pharmaceutically acceptable compositions can be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions can be formulated in an ointment such as petrolatum.

Pharmaceutically acceptable compositions of this invention can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations can be administered with or without food. In some embodiments, pharmaceutically acceptable compositions of this invention are administered without food. In other embodiments, pharmaceutically acceptable compositions of this invention are administered with food.

The amount of compounds of the present invention that can be combined with the carrier materials to produce a composition in a single dosage form varies depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient depends upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition also depends upon the particular compound in the composition.

### Uses of Compounds and Pharmaceutically Acceptable Compositions

According to another embodiment, the invention provides a method for treating a disease or disorder associated with USP1 in a patient, comprising administering to the patient a compound of this invention, or a pharmaceutically acceptable derivative thereof, or a pharmaceutical composition thereof. In some embodiments, the invention provides a use of a compound of this invention, or a pharmaceutically acceptable derivative thereof, or a pharmaceutical composition thereof, in the manufacture of a medicament for the treatment of a disease or disorder associated with USP1. In some embodiments, the invention provides a use of a compound of this invention, or a pharmaceutically acceptable derivative thereof, or a pharmaceutical composition thereof, for the treatment of a disease or disorder associated with USP1. In some embodiments, a disease or disorder associated with USP1 is a proliferative disease, such as cancers as described herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example, to prevent or delay their recurrence.

As used herein, a “proliferative disease” refers to a disease that occurs due to abnormal growth or extension by the multiplication of cells (Walker, Cambridge Dictionary of Biology, Cambridge University Press: Cambridge, UK, 1990). A proliferative disease can be associated with: 1) the pathological proliferation of normally quiescent cells; 2) the pathological migration of cells from their normal location (e.g., metastasis of neoplastic cells); 3) the pathological expression of proteolytic enzymes, such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

### Cancer

The cancer or proliferative disorder or tumor to be treated using the compounds and methods and uses described herein include, but are not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

In some embodiments of the methods and uses described herein, the cancer is lung cancer, thyroid cancer, ovarian cancer, colorectal cancer, prostate cancer, cancer of the pancreas, cancer of the esophagus, liver cancer, breast cancer, skin cancer, or mesothelioma. In some embodiments, the cancer is lung cancer, thyroid cancer, ovarian cancer, colorectal cancer, prostate cancer, cancer of the pancreas, cancer of the esophagus, liver cancer, breast cancer, skin cancer, or mesothelioma, sarcoma, or epithelioid hemangioendothelioma (EHE). In some embodiments, the cancer is mesothelioma, such as malignant mesothelioma. In some embodiments, the cancer is EHE.

In some embodiments, cancer includes, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g., Grade I-Pilocytic Astrocytoma, Grade II-Low-grade Astrocytoma, Grade III-Anaplastic Astrocytoma, or Grade IV-Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (VIPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also http s://clinicaltrials.gov/ct2/show/study/NCT0240886; http s://clinicaltrials.gov/ct2/show/NCT02426892)

In some embodiments, a cancer is melanoma cancer. In some embodiments, a cancer is breast cancer. In some embodiments, a cancer is lung cancer. In some embodiments, a cancer is small cell lung cancer (SCLC). In some embodiments, a cancer is non-small cell lung cancer (NSCLC).

The compounds and compositions, according to the methods of the present invention, can be administered using any amount and any route of administration effective for treating or lessening the severity of a cancer or tumor. The exact amount required varies from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention is decided by the attending physician within the scope of sound medical judgment. The specific effective dose level for any particular patient or organism depends upon a variety of factors, including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The terms “patient” or “subject”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention can be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type can also be employed as fillers in soft and hard-filled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragées, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Co-Administration with One or More Other Therapeutic Agent(s)

Depending upon the particular condition, or disease, to be treated, additional therapeutic agents that are normally administered to treat that condition, can also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention can also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides, or in addition, be administered especially for tumor therapy in combination with chemotherapy, radiotherapy, immunotherapy, phototherapy, surgical intervention, or a combination of these. Long-term therapy is equally possible, as is adjuvant therapy in the context of other treatment strategies, as described above. Other possible treatments are therapy to maintain the patient's status after tumor regression, or even chemopreventive therapy, for example in patients at risk.

One or more other therapeutic agent(s) may be administered separately from a compound or composition of the invention, as part of a multiple dosage regimen. Alternatively, one or more other therapeutic agent(s) may be part of a single dosage form, mixed together with a compound of this invention in a single composition. If administered as a multiple dosage regime, one or more other therapeutic agent(s) and a compound or composition of the invention can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent(s) and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent as described above) that may be combined with the carrier materials to produce a single dosage form will vary depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention may act synergistically. Therefore, the amount of the one or more other therapeutic agent(s) in such compositions may be less than that required in a monotherapy utilizing only that therapeutic agent. In such compositions a dosage of between 0.01-1,000 μg/kg body weight/day of the one or more other therapeutic agent(s) can be administered.

The amount of one or more other therapeutic agent present in the compositions of this invention may be no more than the amount that would normally be administered in a composition comprising that therapeutic agent as the only active agent. Preferably the amount of one or more other therapeutic agent(s) in the presently disclosed compositions will range from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Other Therapeutic Agents

In some embodiments, one or more other therapeutic agent is a Poly ADP ribose polymerase (PARP) inhibitor. In some embodiments, a PARP inhibitor is selected from olaparib (LYNPARZA®, AstraZeneca); rucaparib (RUBRACA®, Clovis Oncology); niraparib (ZEJULA®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (ZOLINZA®, Merck); romidepsin (ISTODAX®, Celgene); panobinostat (FARYDAK®, Novartis); belinostat (BELEODAQ®, Spectrum Pharmaceuticals); entinostat (SNDX-275, Syndax Pharmaceuticals) (NCT00866333); and chidamide (EPIDAZA®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (IBRANCE®, Pfizer); ribociclib (KISQALI®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (ZYDELIG®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells.

In some embodiments, a platinum-based therapeutic is selected from cisplatin (PLATINOL®, Bristol-Myers Squibb); carboplatin (PARAPLATIN®, Bristol-Myers Squibb; also, Teva; Pfizer); oxaliplatin (ELOXITIN® Sanofi-Aventis); nedaplatin (AQUPLA®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (TAXOL®, Bristol-Myers Squibb), docetaxel (TAXOTERE®, Sanofi-Aventis; DOCEFREZ®, Sun Pharmaceutical), albumin-bound paclitaxel (ABRAXANE®; Abraxis/Celgene), cabazitaxel (JEVTANA®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, YONDELIS®, Janssen Oncology), mechlorethamine (alkylating agent, VALCHLOR®, Aktelion Pharmaceuticals); vincristine (ONCOVIN®, Eli Lilly; VINCASAR®, Teva Pharmaceuticals; MARQIBO®, Talon Therapeutics); temozolomide (prodrug to alkylating agent 5-(3-methyltriazen-1-yl)-imidazole-4-carboxamide (MTIC) TEMODAR®, Merck); cytarabine injection (ara-C, antimetabolic cytidine analog, Pfizer); lomustine (alkylating agent, CEENU®, Bristol-Myers Squibb; GLEOSTINE®, NextSource Biotechnology); azacitidine (pyrimidine nucleoside analog of cytidine, VIDAZA®, Celgene); omacetaxine mepesuccinate (cephalotaxine ester) (protein synthesis inhibitor, SYNRIBO®; Teva Pharmaceuticals); asparaginase Envinia chrysanthemi (enzyme for depletion of asparagine, ELSPAR®, Lundbeck; ERWINAZE®, EUSA Pharma); eribulin mesylate (microtubule inhibitor, tubulin-based antimitotic, HALAVEN®, Eisai); cabazitaxel (microtubule inhibitor, tubulin-based antimitotic, JEVTANA®, Sanofi-Aventis); capacetrine (thymidylate synthase inhibitor, XELODA®, Genentech); bendamustine (bifunctional mechlorethamine derivative, believed to form interstrand DNA cross-links, TREANDA®, Cephalon/Teva); ixabepilone (semi-synthetic analog of epothilone B, microtubule inhibitor, tubulin-based antimitotic, IXEMPRA®, Bristol-Myers Squibb); nelarabine (prodrug of deoxyguanosine analog, nucleoside metabolic inhibitor, ARRANON®, Novartis); clorafabine (prodrug of ribonucleotide reductase inhibitor, competitive inhibitor of deoxycytidine, CLOLAR®, Sanofi-Aventis); and trifluridine and tipiracil (thymidine-based nucleoside analog and thymidine phosphorylase inhibitor, LONSURF 0, Taiho Oncology).

In some embodiments, one or more other therapeutic agent is a kinase inhibitor or VEGF-R antagonist. In some embodiments, one or more other therapeutic agent is a MEK inhibitor. As used herein, a “MEK inhibitor” refers to any inhibitor or blocker or antagonist that binds to and/or inhibits mitogen-activated protein kinase enzymes MEK1 and/or MEK2. In some embodiments, an MEK inhibitor is selected from those as described in Cheng et al., “Current Development Status of MEK Inhibitors,” Molecules 2017, 22, 1551, the contents of which are incorporated herein by reference in its entirety. In certain embodiments, the MEK inhibitor is selected from binimetinib (MEK162, ARRY-438162, ARRAY BIOPHARMA INC.), cobimetinib(COTELLIC®, Exelexis/Genentech/Roche), refametinib (BAY 86-9766, RDEA119; Bayer AG), selumetinib (AZD6244, ARRY-142886; ASTRAZENECA), trametinib(MEKINIST®, Novartis), mirdametinib(PD-0325901, Spring Works Therapeutics), pimasertib (AS703026, MSC1936369B, Merck KGaA)or a pharmaceutically acceptable salt and/or solvate of any of the foregoing. In certain embodiments, the second anti-cancer agent is binimetinib, cobimetinib, selumetinib, trametinib, mirdametinib, pimasertib, or a pharmaceutically acceptable salt and/or solvate of any of the foregoing. Other examples of MEK inhibitors for use as an other therapeutic agent in the methods and uses described herein include, but are not limited to. E6201 (Eisai Co Ltd./Strategia Theraputics), GDC-0623 (RG 7421, Genentech, Inc.), CH5126766 (R05126766, Chugai 232 Pharmaceutical Co., Roche), HL-085 (Shanghai Kechow Pharma, Inc.), SHR7390 (FEENGRUI MEDICINE), TQ-B3234 (CHIATAI TIANQING), CS-3006 (CSTONE Pharmaceuticals), FCN-159 (FosunPharmaceuticals), VS-6766 (Verastem Oncology), and EMI-1-104 (Immuneering Corp.). Other examples of MFK inhibitors for use as second anti-cancer agents in the methods and uses described herein include, but are not limited to, those described in WO2005/121142, WO2014/169843, WO2016/035008, WO2016/168704, WO2020/125747 WO2021/142144, WO2021/142345, WO2021/149776, the contents of each of which are herein incorporated by reference in their entireties.

In some embodiments, one or more other therapeutic agent is an EGER inhibitor. As used herein, an “EGER inhibitor” refers to any inhibitor or blocker or antagonist that binds to and/or inhibits epidermal growth factor receptor (EGFR). In some embodiments, an EGFR inhibitor is selected from those as described in Ayati et at., “A review on progression of epidermal growth factor receptor (EGFR) inhibitors as an efficient approach in cancer targeted therapy,” Bioorganic Chemistry 2020, 99: 103811, the contents of which are incorporated herein by reference in its entirety. In some embodiments, an EGFR inhibitor is selected from cetuximab, necitumumab, panitumumab, zalutumumab, nimotuzumab, and matuzumab. In some embodiments, an EGFR inhibitor is cetuximab. In some embodiments, an EGFR inhibitor is necitumumab. In some embodiments, an EGFR inhibitor is panitumumab. In some embodiments, an EGFR inhibitor is zalutumumab. In some embodiments, an EGFR inhibitor is nimotuzumab. In some embodiments, an EGFR inhibitor is matuzumab.

In some embodiments, an EGFR inhibitor is selected from osimertinib, gefitinib, erlotinib, lapatinib, neratinib, vandetanib, afatinib, brigatinib, dacomitinib, and icotinib. In some embodiments, an EGFR inhibitor is Osimertinib. In some embodiments, an EGFR inhibitor is gefitinib. In some embodiments, an EGFR inhibitor is erlotinib. In some embodiments, an EGFR inhibitor is lapatinib. In some embodiments, an EGFR inhibitor is neratinib. In some embodiments, an EGFR inhibitor is vandetanib. In some embodiments, an EGFR inhibitor is afatinib. In some embodiments, an EGFR inhibitor is brigatinib. In some embodiments, an EGFR inhibitor is dacomitinib. In some embodiments, an EGFR inhibitor is icotinib.

In some embodiments, an EGFR inhibitor is a “1st generation EGFR tyrosine kinase inhibitor” (1^{st} generation TKI). A 1″ generation TKI refers to reversible EGFR inhibitors, such as gefitinib and erlotinib, which are effective in first-line treatment of NSCLC harboring EGFR activating mutations such as deletions in exon 19 and exon 21 L858R mutation.

In some embodiments, an EGFR inhibitor is a “2nd generation EGFR tyrosine kinase inhibitor” (2^{nd} generation TKI). A 2n d generation TKI refers to covalent irreversible EGFR inhibitors, such as afatinib and dacomitib, which are effective in first-line treatment of NSCLC harboring EGFR activating mutations such as deletions in exon 19 and exon 21 L858R mutation.

In some embodiments, an EGFR inhibitor is a “3rd generation EGFR tyrosine kinase inhibitor” (3rd generation TKI). A 3rd generation TKI refers to covalent irreversible EGFR inhibitors, such as osimertinib and lazertinib, which are selective to the EGFR activating mutations, such as deletions in exon 19 and exon 21 L858R, alone or in combination with T790M mutation, and have lower inhibitory activity against wild-type EGFR.

In some embodiment, one or more other therapeutic agent is selected from approved VEGF inhibitors and kinase inhibitors useful in the present invention include: bevacizumab (AVASTIN®, Genentech/Roche) an anti-VEGF monoclonal antibody; ramucirumab (CYRANIZA®, Eli Lilly), an anti-VEGFR-2 antibody and ziv-aflibercept, also known as VEGF Trap (ZALTRAP®; Regeneron/Sanofi). VEGFR inhibitors, such as regorafenib (STIVARGA®, Bayer); vandetanib (CAPRELSA®, AstraZeneca); axitinib (INLYTA®, Pfizer); and lenvatinib (LENVIMA®, Eisai); Raf inhibitors, such as sorafenib (NEXAVAR®, Bayer AG and Onyx); dabrafenib (TAFINLAR®, Novartis); and vemurafenib (ZELBORAF®, Genentech/Roche); MEK inhibitors, such as cobimetanib (COTELLIC®, Exelexis/Genentech/Roche); trametinib (MEKINIST®, Novartis); Bcr-Abl tyrosine kinase inhibitors, such as imatinib (GLEEVEC®, Novartis); nilotinib (TASIGNA®, Novartis); dasatinib (SPRYCEL®, BristolMyersSquibb); bosutinib (BOSULIF®, Pfizer); and ponatinib (INCLUSIG®, Ariad Pharmaceuticals); Her2 and EGFR inhibitors, such as gefitinib (IRESSA®, AstraZeneca); erlotinib (TARCEEVA®, Genentech/Roche/Astellas); lapatinib (TYKERB®, Novartis); afatinib (GILOTRIF®, Boehringer Ingelheim); osimertinib (targeting activated EGFR, TAGRISSO®, AstraZeneca); and brigatinib (ALUNBRIG®, Ariad Pharmaceuticals); c-Met and VEGFR2 inhibitors, such as cabozanitib (COMETRIQ®, Exelexis); and multikinase inhibitors, such as sunitinib (SUTENT®, Pfizer); pazopanib (VOTRIENT®, Novartis); ALK inhibitors, such as crizotinib (XALKORI®, Pfizer); ceritinib (ZYKADIA®, Novartis); and alectinib (ALECENZa®, Genentech/Roche); Bruton's tyrosine kinase inhibitors, such as ibrutinib (IMBRUVICA®, Pharmacyclics/Janssen); and Flt3 receptor inhibitors, such as midostaurin (RYDAPT®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (SUPECT®, IY5511, Il-Yang Pharmaceuticals, S. Korea); ruxolitinib (JAKAFI®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (AFINITOR®, Novartis); temsirolimus (TORISEL®, Pfizer); and sirolimus (RAPAMUNE®, Pfizer).

In some embodiments, one or more other therapeutic agent is a proteasome inhibitor. Approved proteasome inhibitors useful in the present invention include bortezomib (VELCADE®, Takeda); carfilzomib (KYPROLIS®, Amgen); and ixazomib (NINLARO®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (LARTRUVO®; Eli Lilly). Approved EGFR antagonists which may be used in the present invention include cetuximab (ERBITUX®, Eli Lilly); necitumumab (PORTRAZZA®, Eli Lilly), panitumumab (VECTIBIX®, Amgen); and osimertinib (targeting activated EGFR, TAGRISSO®, AstraZeneca).

In some embodiments, one or more other therapeutic agent is an aromatase inhibitor. In some embodiments, an aromatase inhibitor is selected from exemestane (AROMASIN®, Pfizer); anastazole (ARIMIDEX®, AstraZeneca) and letrozole (FEMARA®, Novartis).

In some embodiments, one or more other therapeutic agent is an antagonist of the hedgehog pathway. Approved hedgehog pathway inhibitors which may be used in the present invention include sonidegib (ODOMZO®, Sun Pharmaceuticals); and vismodegib (ERIVEDGE®, Genentech), both for treatment of basal cell carcinoma.

In some embodiments, one or more other therapeutic agent is a folic acid inhibitor. Approved folic acid inhibitors useful in the present invention include pemetrexed (ALIMTA®, Eli Lilly).

In some embodiments, one or more other therapeutic agent is a CC chemokine receptor 4 (CCR4) inhibitor. CCR4 inhibitors being studied that may be useful in the present invention include mogamulizumab (POTELIGEO®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (RITUXAN®, Genentech/BiogenIdec); ofatumumab (anti-CD20, ARZERRA®, GlaxoSmithKline); obinutuzumab (anti-CD20, GAZYVA®, Genentech), ibritumomab (anti-CD20 and Yttrium-90, ZEVALIN®, Spectrum Pharmaceuticals); daratumumab (anti-CD38, DARZALEX®, Janssen Biotech), dinutuximab (anti-glycolipid GD2, UNITUXIN®, United Therapeutics); trastuzumab (anti-HER2, HERCEPTIN®, Genentech); ado-trastuzumab emtansine (anti-HER2, fused to emtansine, KADCYLA®, Genentech); and pertuzumab (anti-HER2, PERJETA®, Genentech); and brentuximab vedotin (anti-CD30-drug conjugate, ADCETRIS®, Seattle Genetics).

In some embodiments, one or more other therapeutic agent is a topoisomerase inhibitor. Approved topoisomerase inhibitors useful in the present invention include irinotecan (ONIVYDE®, Merrimack Pharmaceuticals); topotecan (HYCAMTIN®, GlaxoSmithKline). Topoisomerase inhibitors being studied which may be used in the present invention include pixantrone (PIXUVRI®, CTI Biopharma).

In some embodiments, one or more other therapeutic agent is an inhibitor of anti-apoptotic proteins, such as BCL-2. Approved anti-apoptotics which may be used in the present invention include venetoclax (VENCLEXTA®, AbbVie/Genentech); and blinatumomab (BLINCYTO®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (XTANDI®, Astellas/Medivation); approved inhibitors of androgen synthesis include abiraterone (ZYTIGA®, Centocor/Ortho); approved antagonist of gonadotropin-releasing hormone (GnRH) receptor (degaralix, FIRMAGON®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (EVISTA®, Eli Lilly).

In some embodiments, one or more other therapeutic agent is an inhibitor of bone resorption. An approved therapeutic which inhibits bone resorption is Denosumab (XGEVA®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (ZOMETA®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGF-β). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA-formerly MSB0011459X), which is a bispecific, anti-PD-L1/TGF-β trap compound (NCT02699515); and (NCT02517398). M7824 is comprised of a fully human IgG1 antibody against PD-L1 fused to the extracellular domain of human TGF-beta receptor II, which functions as a TGF-β “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agents is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (TEMODAL c)); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name AROMASIN™. Formestane is marketed under the trade name LENTARON™. Fadrozole is marketed under the trade name AFEMA™. Anastrozole is marketed under the trade name ARJIIVIIDEX™. Letrozole is marketed under the trade names FEMARA™ or FEMAr™. Aminoglutethimide is marketed under the trade name ORIMETEN™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name NOLVADEX™. Raloxifene hydrochloride is marketed under the trade name EVISTA™. Fulvestrant can be administered under the trade name FASLODEX™Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (CASODEX™). The term “gonadorelin agonist” as used herein includes, but is not limited to abarelix, goserelin, and goserelin acetate. Goserelin can be administered under the trade name ZOLADEX™

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark CAMPTOSAR™. Topotecan is marketed under the trade name HYCAMPTIN™.

The term “topoisomerase II inhibitor” as used herein includes, but is not limited to the anthracyclines such as doxorubicin (including liposomal formulation, such as CAELYX™) daunorubicin, epirubicin, idarubicin and nemorubicin, the anthraquinones mitoxantrone and losoxantrone, and the podophillotoxines etoposide and teniposide. Etoposide is marketed under the trade name ETOPOPHOS™. Teniposide is marketed under the trade name VM 26-Bristol Doxorubicin is marketed under the trade name ACRIBLASTIN™ or ADRIAMYCIN™. Epirubicin is marketed under the trade name FARIVIORUBICIN™. Idarubicin is marketed. under the trade name ZAVEDOS™. Mitoxantrone is marketed under the trade name NOVANTRON™.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name TAXOL™. Docetaxel is marketed under the trade name TAXOTERE™. Vinblastine sulfate is marketed under the trade name VINBLASTIN R.P™. Vincristine sulfate is marketed under the trade name FARMISTIN™.

The term “alkylating agent” as used herein includes, but is not limited to, cyclophosphamide, ifosfamide, melphalan or nitrosourea (BCNU or Gliadel). Cyclophosphamide is marketed under the trade name CYCLOSTIN™. Ifosfamide is marketed under the trade name HOLOXAN™

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA™. Gemcitabine is marketed under the trade name GEMZAR™.

The term “platin compound” as used herein includes, but is not limited to, carboplatin, cis-platin, cisplatinum and oxaliplatin. Carboplatin can be administered, e.g., in the form as it is marketed, e.g., under the trademark CARBOPLAT™. Oxaliplatin can be administered, e.g., in the form as it is marketed, e.g. under the trademark ELOXATIN™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P 13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (HERCEPTIN™), cetuximab (ERBITUX™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β,p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bc1-2), including but not limited to ABT-199, ABT-731, ABT-737, apogossypol, Ascenta's pan-Bcl-2 inhibitors, curcumin (and analogs thereof), dual Bc1-2/Bc1-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (THALOMID™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g., inhibitors of phosphatase 1, phosphatase 2A, or CDC25, such as okadaic acid or a derivative thereof.

Compounds which induce cell differentiation processes include, but are not limited to, retinoic acid, α-γ- or δ-tocopherol or α-γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (CELEBREX™), rofecoxib (VIOXX™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name DIDRONEL™. Clodronic acid is marketed under the trade name BONEFOS™. Tiludronic acid is marketed under the trade name Skelid™. Pamidronic acid is marketed under the trade name AREDIA™. Alendronic acid is marketed under the trade name FOSAMAX™. Ibandronic acid is marketed under the trade name BONDRANAT™. Risedronic acid is marketed under the trade name ACTONEL™. Zoledronic acid is marketed under the trade name ZOMETA™. The term “mTOR inhibitors” relates to compounds which inhibit the mammalian target of rapamycin (mTOR) and which possess antiproliferative activity such as sirolimus (RAPAMUNE®), everolimus (CERTICAN™), CC₁₋₇₇₉ and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (ZARNESTRA™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (VELCADE™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (HERCEPTIN™), Trastuzumab-DM1, erbitux, bevacizumab (AVASTIN™), rituximab (RITUXAN (9), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-di one derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; ANGIOSTATIN™; ENDOSTATIN™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (AVASTIN™).

Photodynamic therapy as used herein refers to therapy which uses certain chemicals known as photosensitizing compounds to treat or prevent cancers. Examples of photodynamic therapy include treatment with compounds, such as VISUDYNE™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

Exemplary Immuno-Oncology agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTOR, LIGHT, DcR3, HVEM, VEGUTL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIRL TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonist of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13/69264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-C SF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-de1ta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June et al.; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors& pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that can be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhlL-15). rhlL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhlL-12). In some embodiments, an IL-15 based immunotherapeutic is heterodimeric IL-15 (hetlL-15, Novartis/Admune), a fusion complex composed of a synthetic form of endogenous IL-15 complexed to the soluble IL-15 binding protein IL-15 receptor alpha chain (IL15:ₛIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhlL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+(4) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GALS, LAG3, VISTA, KIR, 2B4, CD 160 and CGEN-15049. Illustrative immune checkpoint inhibitors include, but are not limited to, Tremelimumab (CTLA-4 blocking antibody), anti-0X⁴⁰, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA0). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (KEYTRUDA0), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include 0X⁴⁰ agonists. OX⁴⁰ agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### Exemplification

The following examples are intended to illustrate the invention and are not to be construed as being limitations thereon. Unless otherwise stated, one or more tautomeric forms of compounds of the examples described hereinafter may be prepared in situ and/or isolated. All tautomeric forms of compounds of the examples described hereafter should be considered to be disclosed. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

### Example 1: Synthesis of Exemplary Compounds

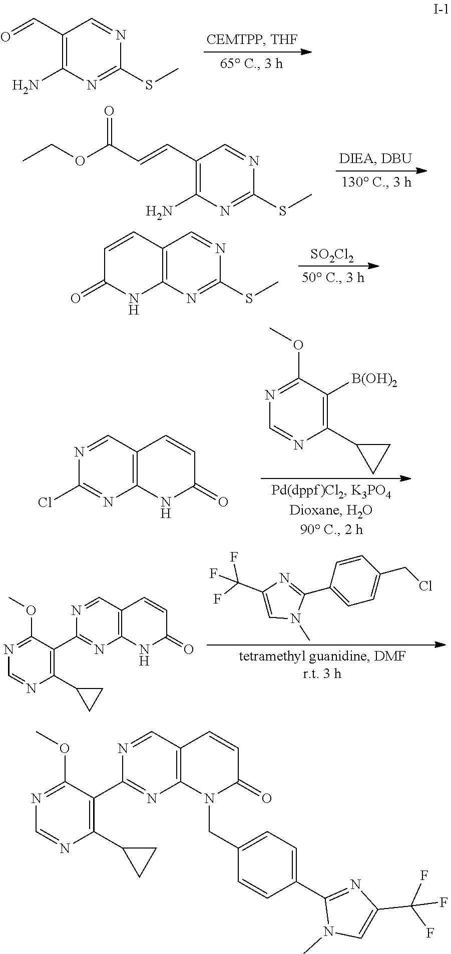

### Step 1: Ethyl (2E)-3-[4-Amino-2-(Methylsulfanyl)Pyrimidin-5-Yl]Prop-2-Enoate

To a stirred solution of 4-amino-2-(methylsulfanyl)pyrimidine-5-carbaldehyde (10 g, 59 mmol, 1 equiv) in THF (150 mL) was added ethyl 2-(triphenyl-lambda5-phosphanylidene)acetate (22.65 g, 65 mmol, 1.1 equiv) at room temperature. The resulting mixture was stirred for 3 h at 65° C. The resulting mixture was concentrated under reduced pressure. The residue was purified by reverse flash chromatography (C18 silica gel column, eluting with 0% to 100% MeCN/water (0.1% TFA) in 10 min with UV detector (254 nm)) to yield ethyl (2E)-3-[4-amino-2-(methylsulfanyl)pyrimidin-5-yl]prop-2-enoate (9.0 g, 63.6%) as a light yellow solid. LC-MS (ESI) m/z, 240.2 [M+H]

### Step 2: 2-(Methylsulfanyl)-811-Pyrido[2,3-d]Pyrimidin-7-One

To a stirred solution of ethyl (2E)-3-[4-amino-2-(methylsulfanyl)pyrimidin-5-yl]prop-2-enoate (10 g, 41.79 mmol, 1 equiv) in DIEA (100 mL) was added DBU (9.54 g, 62.68 mmol, 1.5 equiv) at room temperature. The resulting mixture was stirred for 3 h at 130° C. The resulting mixture was concentrated under reduced pressure. The residue was purified by reverse flash chromatography (C18 silica gel column, eluting with 0% to 100% MeCN/water (0.1% FA) in 15 min with UV detector (254 nm)) to yield 2-(methylsulfanyl)-8H-pyrido[2,3-d]pyrimidin-7-one (5.5 g, 68.11%) as a light yellow solid. LC-MS (ESI) m/z 194.2 [M+H]

### Step 3: 2-Chloro-811-Pyrido[2,3-d]Pyrimidin-7-One

A solution of 2-(methylsulfanyl)-8H-pyrido[2,3-d]pyrimidin-7-one (6 g, 31.053 mmol, 1 equiv) in SO₂Cl₂ (60 mL) was stirred at 50° C. for 3 h. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to yield 2-chloro-8H-pyrido[2,3-d]pyrimidin-7-one (3 g, 53.21%) as an off-white solid. LC-MS (ESI) m/z 182.5 [M+H]

### Step 4: 2-(4-Cyclopropyl-6-Methoxypyrimidin-5-Yl)-811-Pyrido[2,3-d]Pyrimidin-7-One

To a stirred solution of 2-chloro-8H-pyrido[2,3-d]pyrimidin-7-one (3 g, 16.522 mmol, 1 equiv) and 4-cyclopropyl-6-methoxypyrimidin-5-ylboronic acid (4.81 g, 24.7 mmol, 1.5 equiv) in 1,4-dioxane (60 mL) and H₂O (12 mL) was added Pd(dppf)Cl₂ (2.42 g, 3.3 mmol, 0.2 equiv) and K₃PO₄ (8.77 g, 41.305 mmol, 2.5 equiv) at room temperature. The resulting mixture was stirred for 2 h at 90° C. under nitrogen atmosphere. The resulting mixture was filtered, the filter cake was washed with 1,4-dioxane (3*50 mL). The filtrate was concentrated under reduced pressure. The residue was purified by reverse flash chromatography (C18 silica gel column, mobile phase, eluting with 0% to 100% MeCN/water (0.1% FA) in 10 min with UV detector (254 nm)) to yield 2-(4-cyclopropyl-6-methoxypyrimidin-5-yl)-8H-pyrido[2,3-d]pyrimidin-7-one (2.3 g, 47.14%) as an off-white solid. LC-MS (ESI) m/z 296.3 [M+H]

### Step 5: 2-(4-Cyclopropyl-6-Methoxypyrimidin-5-Yl)-8-({4-[1-Methyl-4-(Trifluoromethyl)Imidazol-2-Yl]Phenyl}Methyl)Pyrido[2,3-d]Pyrimidin-7-One

To a stirred solution of 2-(4-cyclopropyl-6-methoxypyrimidin-5-yl)-8H-pyrido[2,3-d]pyrimidin-7-one (1.9 g, 6.4 mmol, 1.1 equiv) and 2-[4-(chloromethyl)phenyl]-1-methyl-4-(trifluoromethyl)imidazole (1.61 g, 5.8 mmol, 1 equiv) in DMF (20 mL) was added 1,1,3,3-tetramethylguanidine (1.01 g, 8.774 mmol, 1.5 equiv) at room temperature. The resulting mixture was stirred for 3 h at room temperature under nitrogen atmosphere. The resulting mixture was extracted with EtOAc (3×100 mL). The combined organic layers were washed with water (3×100 mL), dried over anhydrous Na₂SO₄ and concentrated under reduced pressure. The residue was purified by reverse flash chromatography (C18 silica gel column, eluting with 0% to 100% MeCN/water (0.1% FA) in 20 min with UV detector (254 nm)) to yield 2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (2.16 g, 69.22%) as an off-white solid. ¹H NMR (400 MHz, DMSO-d6) δ 9.29 (s, 1H), 8.69 (s, 1H), 8.16 (d, J=9.6 Hz, 1H), 7.91 (d, J=1.4 Hz, 1H), 7.68-7.58 (m, 2H), 7.40 (d, J=8.2 Hz, 2H), 6.90 (d, J=9.5 Hz, 1H), 5.57 (s, 2H), 3.78 (d, J=36.0 Hz, 6H), 1.72 (m, 1H), 1.01 (m, 2H), 0.77 (m, 2H). LC-MS(ESI) m/z 534.0[M+H]

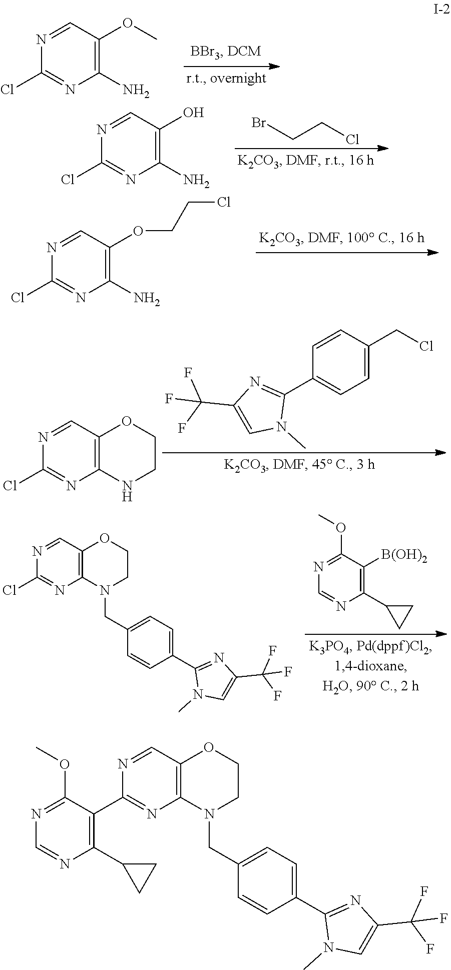

### Step 1: 4-Amino-2-Chloropyrimidin-5-Ol

To a stirred mixture of 2-chloro-5-methoxypyrimidin-4-amine (1 g, 6.27 mmol, 1 equiv) in CH₂ Cl₂ (10 mL, 157.306 mmol, 25.10 equiv) were added BBr₃ (9.1 mL, 96.26 mmol, equiv) at room temperature. The resulting mixture was stirred for overnight at room temperature. The reaction was quenched by the addition of MeOH (4 mL) at 0° C. The precipitated solids were collected by filtration and washed with MeOH (3×5 mL). The resulting solid was dried under vacuum to afford 4-amino-2-chloropyrimidin-5-ol (0.7 g, 76.74%) as a white solid. The crude product mixture was used in the next step directly without further purification. LC-MS (ESI) m/z 146 [M+H].

### Step 2: 2-Chloro-5-(2-Chloroethoxy)Pyrimidin-4-Amine

To a stirred solution of 4-amino-2-chloropyrimidin-5-ol (732 mg, 5.029 mmol, 1 equiv) and K₂CO₃ (4170.36 mg, 30.174 mmol, 6 equiv) in DMF(10 ml, 129.215 mmol, 25.69 equiv) were added 1-bromo-2-chloroethane (2163.71 mg, 15.087 mmol, 3 equiv) dropwise at room temperature under nitrogen atmosphere. The final reaction mixture was stirred for overnight at room temperature. The resulting mixture was extracted with EtOAc (3×10 mL). The combined organic layers were washed with brine (3×10 mL), dried over anhydrous Na₂SO₄, filtered and concentrated under reduced pressure to afford 2-chloro-5-(2-chloroethoxy)pyrimidin-4-amine (450 mg, 43.01%) as an off-white solid. The crude product mixture was used in the next step directly without further purification. LC-MS (ESI) m/z 208[M+H]

### Step 3: 2-Chloro-611,711,811-Pyrimido[5,4-b][1,4]Oxazine

To a stirred solution of 2-chloro-5-(2-chloroethoxy)pyrimidin-4-amine (450 mg, 2.163 mmol, 1 equiv) in DMF (5 ml, 64.608 mmol, 24.63 equiv) was added K₂CO₃ (1195.77 mg, 8.652 mmol, 4 equiv) in portions at room temperature under nitrogen atmosphere. The final reaction mixture was stirred for overnight at 100° C. The resulting mixture was extracted with EtOAc (3×20 mL). The combined organic layers were washed with brine (3×10 mL), dried over anhydrous Na₂SO₄, filtered and concentrated under reduced pressure. The residue was purified by silica gel column chromatography, (eluting with PE/EA (5:1)) to afford 2-chloro-6H,7H,8H-pyrimido[5,4-b][1,4]oxazine (300 mg, 80.83%) as an off-white solid. LC-MS(ESI) m/z 172 [M+H]

### Step 4: 2-[4-({2-Chloro-6H,7H-Pyrimido[5,4-b][1,4]Oxazin-8-Yl}Methyl)Phenyl]-1-Methyl-4-(Trifluoromethyl)Imidazole

To a stirred solution of 2-chloro-6H,7H,8H-pyrimido[5,4-b][1,4]oxazine (280 mg, 1.632 mmol, 2.5 equiv) and 2-[4-(chloromethyl)phenyl]-1-methyl-4-(trifluoromethyl)imidazole (179.29 mg, 0.653 mmol, 1 equiv) in DMF (5 ml, 64.608 mmol, 39.59 equiv) was added K₂CO3 (270.64 mg, 1.958 mmol, 3 equiv) in portions at room temperature under nitrogen atmosphere. The final reaction mixture was stirred for 3h at 45° C. The reaction was quenched with Water at 0° C. The resulting mixture was extracted with EtOAc (3×20 mL). The combined organic layers were washed with brine (1×10 mL), dried over anhydrous Na₂SO₄, filtered and concentrated under reduced pressure. The residue was purified by reverse flash chromatography (C18 silica gel column, eluting with 10% to 100% MeCN/water (10 mmol/L NH₄HCO₃) in 10 min with UV detector (254 nm)) to yield 2-[4-({2-chloro-6H,7H-pyrimido[5,4-b][1,4]oxazin-8-yl}methyl)phenyl]-1-methyl-4-(trifluoromethyl)imidazole (226.1 mg, 84.52%) as an orange solid. LC-MS (ESI) m/z 410 [M+H]

### Step 5: 4-Cyclopropyl-6-Methoxy-5-[8-({4-[1-Methyl-4-(Trifluoromethyl)Imidazo]-2-Yl]Phenyl}Methyl)-611,711-Pyrimido[5,4-b][1,4]Oxazin-2-Yl]Pyrimidine

To a stirred solution of 2-[4-({2-chloro-6H,7H-pyrimido[5,4-b][1,4]oxazin-8-yl}methyl)phenyl]-1-methyl-4-(trifluoromethyl)imidazole (160 mg, 0.390 mmol, 1 equiv) and 4-cyclopropyl-6-methoxypyrimidin-5-ylboronic acid (113.62 mg, 0.585 mmol, 1.5 equiv) in 1,4-dioxane (3 ml, 12.000 mmol, 30.74 equiv) were added K₃PO₄ (207.19 mg, 0.975 mmol, 2.5 equiv) and H₂O (0.3 mL, 16.653 mmol, 42.65 equiv) at room temperature under nitrogen atmosphere. To the above mixture was added Pd(dppf)C12 (57.14 mg, 0.078 mmol, 0.2 equiv) at room temperature. The resulting mixture was stirred for additional 2 h at 90° C. The reaction was quenched with Water at 0° C. The resulting mixture was extracted with EtOAc (3×30 mL). The combined organic layers were washed with brine (1×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% FA) with UV detector (254 nm)) to yield 4-cyclopropyl-6-methoxy-5-[8-({4-[1-methyl-4-(trifluoromethyl)imidazo]-2-yl]phenyl methyl)-6H,7H-pyrimido[5,4-b][1,4]oxazin-2-yl]pyrimidine (76.3 mg, 37.33%) as an off-white solid.

¹H NMR (400 MHz, DMSO-d6) δ 8.66 (s, 1H), 8.15 (s, 1H), 7.94 (d, J=1.4 Hz, 1H), 7.72-7.65 (m, 2H), 7.46 (d, J=8.1 Hz, 2H), 4.96 (s, 2H), 4.35 (s, 3H), 3.88 (s, 3H), 3.77 (s, 3H), 3.69 (s, 2H), 1.86 (s, 1H), 1.05-0.98 (m, 2H), 0.89-0.81 (m, 2H). LC-MS(ESI) m/z 524.20 [M+H]

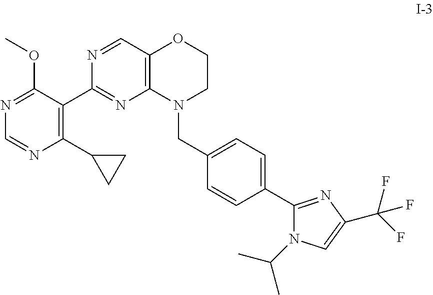 [SMILES: COc1ncnc(C2CC2)c1-c1ncc2c(n1)N(Cc1ccc(-c3nc(C(F)(F)F)cn3C(C)C)cc1)CCO2]

1-3 was synthesized in an analogous way as 1-2

¹H NMR (400 MHz, Chloroform-d) δ 8.72 (s, 1H), 8.19 (s, 1H), 7.57 (s, 2H), 7.47 (s, 1H), 7.41 (s, 2H), 5.11 (s, 2H), 4.53 (s, 1H), 4.36 (s, 2H), 4.07 (s, 3H), 3.72 (s, 2H), 1.93 (s, 1H), 1.49 (d, J=5.5 Hz, 6H), 1.34 (s, 2H), 0.95 (s, 2H). LC-MS (ESI) m/z 552.40 [M+H]

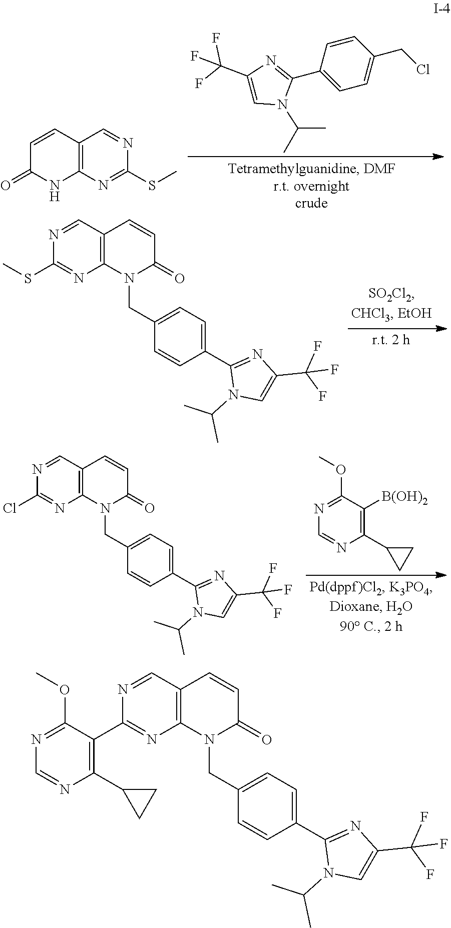

### Step 1: 8-({4-[1-Isopropyl-4-(Trifluoromethyl)Imidazol-2-Yl]Phenyl}Methyl)-2-(Methylsulfanyl)Pyrido[2,3-d]Pyrimidin-7-One

To a stirred solution of 2-(methylsulfanyl)-8H-pyrido[2,3-d]pyrimidin-7-one (200 mg, 1.035 mmol, 1 equiv) and 2-[4-(chloromethyl)phenyl]-1-isopropyl-4-(trifluoromethyl)imidazole (313.35 mg, 1.035 mmol, 1 equiv) in DMF (2 mL) was added 1,1,3,3-tetramethylguanidine (178.83 mg, 1.552 mmol, 1.5 equiv) dropwise at room temperature. The resulting mixture was stirred for overnight at room temperature under nitrogen atmosphere. The reaction was quenched with Water (10 mL) at room temperature. The resulting mixture was extracted with EtOAc (3×20 mL). The combined organic layers were washed with brine (3×10 mL), dried over anhydrous Na₂SO₄, filtered and concentrated under reduced pressure. The crude product was used in the next step directly without further purification. LC-MS (ESI) m/z: 460.1 [M+H]

### Step 2 2-Chloro-8-({4-[1-Isopropyl-4-(Trifluoromethyl)Imidazol-2-Yl]Phenyl}Methyl)Pyrido[2,3-d]Pyrimidin-7-One

To a stirred solution of 8-({4-[1-isopropyl-4-(trifluoromethyl)imidazo]-2-yl]phenyl}methyl)-2-(methylsulfanyl)pyrido[2,3-d]pyrimidin-7-one (150 mg, 0.326 mmol, 1 equiv) in CHCl₃ (1 mL) and EtOH (0.1 mL) was added SO₂Cl₂ (132.17 mg, 0.978 mmol, 3 equiv) dropwise at room temperature. The resulting mixture was stirred for 2 h at room temperature and concentrated under reduced pressure. The residue was purified by reverse flash chromatography (C18 silica gel column, eluting with 10% to 100% MeCN/water (0.1% NH₃H₂O) in 10 min with UV detector (254 nm)) to afford 2-chloro-8-({4-[1-isopropyl-4-(trifluoromethyl)imidazo]-2-yl]phenyl}methyl)pyrido[2,3-d]pyrimidin-7-one (100 mg, 68.40%) as a light brown solid. LC-MS (ESI) m/z: 448.1 [M+H]

### Step 3: 2-(4-Cyclopropyl-6-Methoxypyrimidin-5-Yl)-8-({4-[1-Isopropyl-4-(Trifluoromethyl)Imidazol-2-Yl]Phenyl}Methyl)Pyrido[2,3-d]Pyrimidin-7-One

To a stirred solution of 2-chloro-8-({4-[1-isopropyl-4-(trifluoromethyl)imidazo]-2-yl]phenyl}methyl)pyrido[2,3-d]pyrimidin-7-one (100 mg, 0.223 mmol, 1 equiv) and 4-cyclopropyl-6-methoxypyrimidin-5-ylboronic acid (64.98 mg, 0.335 mmol, 1.5 equiv) in 1,4-dioxane (0.8 mL) and water (0.2 mL) was added Pd(dppf)C12 (32.68 mg, 0.045 mmol, 0.2 equiv) and K₃PO₄ (118.49 mg, 0.557 mmol, 2.5 equiv) at room temperature. The resulting mixture was stirred for 2 h at 90° C. under nitrogen atmosphere. The resulting mixture was filtered; the filter cake was washed with 1,4-dioxane (3×15 mL). The filtrate was concentrated under reduced pressure. The crude product was purified by Prep-HPLC with the following conditions (Column: YMC-Actus Triart C18 ExRS, 30*150 mm, 51 μm; Mobile Phase A: Water(10 mmol/L NH₄HCO₃), Mobile Phase B: ACN; Flow rate: 60 mL/min; Gradient: 55% B to 65% B in 7 min, 65% B; Wave Length: 220 nm; RT 1(min): 5.03) to afford 2-(4-cyclopropyl-6-methoxypyrimidin-5-yl)-8-({4-[1-isopropyl-4-(trifluoromethyl)imidazo]-2-yl]phenyl}methyl)pyrido[2,3-d]pyrimidin-7-one (26.1 mg, 20.42%) as a brown yellow liquid. ¹H NMR (400 MHz, Chloroform-d) δ 9.02 (s, 1H), 8.69 (s, 1H), 7.77 (d, J=9.5 Hz, 1H), 7.61 (d, J=8.0 Hz, 2H), 7.47-7.36 (m, 3H), 6.87 (d, J=9.5 Hz, 1H), 5.71 (s, 2H), 4.51 (p, J=6.7 Hz, 1H), 3.93 (s, 3H), 1.70 (m, 1H), 1.42 (d, J=6.7 Hz, 6H), 1.30-1.15 (m, 2H), 0.86 (m, 2H). LC-MS (ESI) m/z 562.3 [M+H].

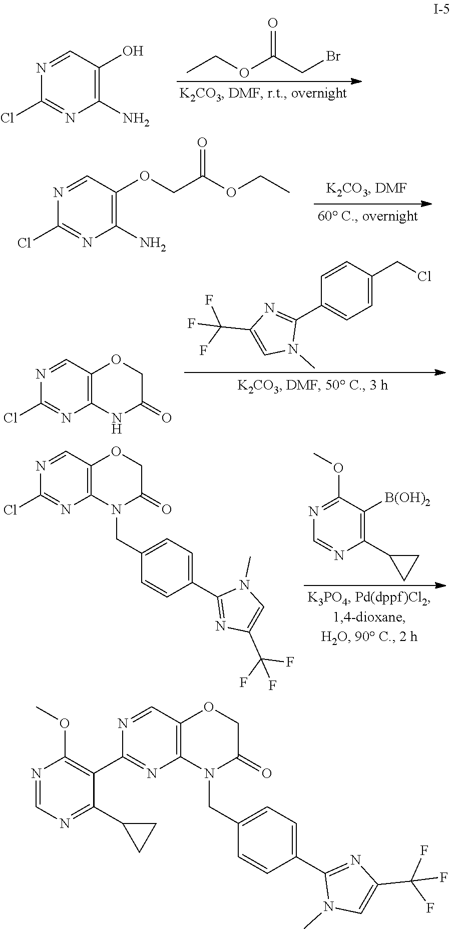

### Step 1: Synthesis of Ethyl 2-1(4-Amino-2-Chloropyrimidin-5-Yl)Oxy]Acetate

To a stirred solution of 4-amino-2-chloropyrimidin-5-ol (511 mg, 3.511 mmol, 1 equiv) and K₂CO₃ (345 mg, 2.496 mmol, 0.71 equiv) in DMF(10 mL, 73.095 mmol, 36.80 equiv) was added ethyl bromoacetate (383 mg, 2.293 mmol, 0.65 equiv) dropwise at room temperature under nitrogen atmosphere. The resulting mixture was stirred for additional overnight at room temperature. The resulting mixture was extracted with EtOAc (3×20 mL). The combined organic layers were washed with brine (3×10 mL), dried over anhydrous Na2SO4, filtered and concentrated under reduced pressure. The resulting mixture was concentrated under vacuum to afford ethyl 2-[(4-amino-2-chloropyrimidin-5-yl)oxy]acetate (461.0 mg, 56.69%) as an off-white solid. The crude product mixture was used in the next step directly without further purification. LC-MS (ESI) m/z 232 [M+H]

### Step 2: 2-Chloro-611,811-Pyrimido[5,4-b][1,4]Oxazin-7-One

To a stirred solution of ethyl 2-[(4-amino-2-chloropyrimidin-5-yl)oxy]acetate (465 mg, 2.007 mmol, 1 equiv) in DMF (8 mL, 103.372 mmol, 51.49 equiv) were added K₂CO₃ (138.72 mg, 1.004 mmol, 0.5 equiv) in portions at room temperature under nitrogen atmosphere. The resulting mixture was stirred for additional overnight at 60° C. The resulting mixture was extracted with EtOAc (3×10 mL). The combined organic layers were washed with brine (3×10 mL), dried over anhydrous Na₂SO₄, filtered and concentrated under reduced pressure. The resulting mixture was concentrated under vacuum to afford 2-chloro-6H,8H-pyrimido[5,4-b][1,4]oxazin-7-one (214.9 mg, 57.69%) as a yellow solid. The crude product mixture was used in the next step directly without further purification. LC-MS (ESI) m/z 186 [M+H]

### Step 3: 2-Chloro-8-({4-[1-Methyl-4-(Trifluoromethyl)Imidazo]-2-Yl]Phenyl}Methyl)-611-Pyrimido[5,4-b][1,4]Oxazin-7-One

To a stirred solution of 2-chloro-6H,8H-pyrimido[5,4-b][1,4]oxazin-7-one (215 mg, 1.159 mmol, 1 equiv) and K₂CO₃ (160 mg, 1.158 mmol, 1.00 equiv) in DMF (4 mL, 51.686 mmol, 44.61 equiv) was added 2-[4-(chloromethyl)phenyl]-1-methyl-4-(trifluoromethyl)imidazole (106 mg, 0.386 mmol, 0.33 equiv) dropwise at room temperature under nitrogen atmosphere. The resulting mixture was stirred for 3 h at 50° C. The resulting mixture was extracted with EtOAc (3×10 mL). The combined organic layers were washed with brine (3×10 mL), dried over anhydrous Na₂SO₄, filtered and concentrated under reduced pressure. The residue was purified by silica gel column chromatography, (eluting with PE/EA (1:1)) to afford 2-chloro-8-({4-[1-methyl-4-(trifluoromethyl)imidazo]-2-yl]phenyl}methyl)-6H-pyrimido[5,4-b][1,4]oxazin-7-one (125 mg, 25.46%) as an off-white solid. LC-MS (ESI) m/z 424 [M+H]

### Step 4: Synthesis of 2-(4-Cyclopropyl-6-Methoxypyrimidin-5-Yl)-8-({4-[1-Methyl-4-(Trifluoromethyl)Imidazo]-2-Yl]Phenyl}Methyl)-611-Pyrimido[5,4-b][1,4]Oxazin-7-One

To a stirred solution of 2-chloro-8-({4-[1-methyl-4-(trifluoromethyl)imidazol-2-yl]phenyl}methyl)-6H-pyrimido[5,4-b][1,4]oxazin-7-one (130 mg, 0.307 mmol, 1 equiv) and 4-cyclopropyl-6-methoxypyrimidin-5-ylboronic acid (89.27 mg, 0.461 mmol, 1.5 equiv) in 1,4-dioxane (2 mL, 8.000 mmol, 26.08 equiv) were added K₃PO₄ (162.79 mg, 0.767 mmol, 2.5 equiv) and H₂O (0.18 mL, 9.992 mmol, 32.57 equiv) at room temperature under nitrogen atmosphere. To the above mixture was added Pd(dppf)C12 (44.89 mg, 0.061 mmol, 0.2 equiv) at room temperature. The resulting mixture was stirred for 3 h at 90° C. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/Water (0.1% FA) with UV detector (254 nm)) to yield 2-(4-cyclopropyl-6-methoxypyrimidin-5-yl)-8-({4-[1-methyl-4-(trifluoromethyl)imidazo]-2-yl]phenyl}methyl)-6H-pyrimido[5,4-b][1,4]oxazin-7-one (80 mg, 48.52%) as an off-white solid. ¹H NMR (400 MHz, DMSO-d6) δ 8.64 (s, 1H), 8.53 (s, 1H), 7.92 (d, J=1.4 Hz, 1H), 7.68-7.61 (m, 2H), 7.46 (d, J=8.2 Hz, 2H), 5.24 (s, 2H), 5.05 (s, 2H), 3.81 (s, 3H), 3.75 (s, 3H), 1.68 (tt, J=8.2, 4.6 Hz, 1H), 1.03-0.95 (m, 2H), 0.77 (dq, J=7.0, 3.4 Hz, 2H). LC-MS (ESI) m/z 538 [M+H]

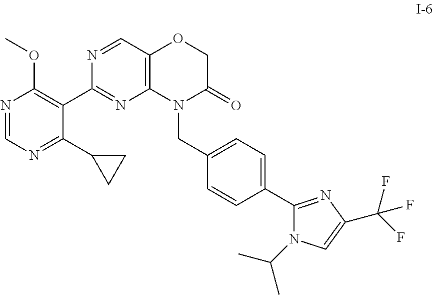 [SMILES: COc1ncnc(C2CC2)c1-c1ncc2c(n1)N(Cc1ccc(-c3nc(C(F)(F)F)cn3C(C)C)cc1)C(=O)CO2]

1-6 was made in an analogous way as 1-5

¹H NMR (400 MHz, DMSO-d6) δ 8.70-8.64 (m, 1H), 8.17 (d, J=8.0 Hz, 1H), 7.53 (q, J=8.4 Hz, 4H), 6.76 (s, 1H), 4.98 (d, J=3.6 Hz, 2H), 4.36 (s, 2H), 3.91-3.86 (m, 3H), 3.72 (s, 2H), 2.35-2.30 (m, 3H), 1.87 (s, 1H), 1.02 (s, 2H), 0.85 (dd, J=8.0, 3.4 Hz, 2H). LC-MS (ESI) m/z 566 [M+H].

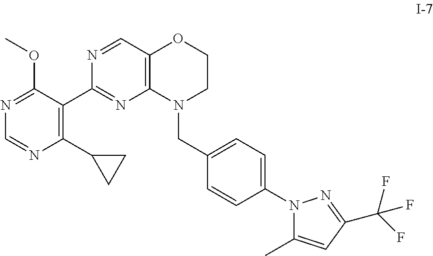 [SMILES: COc1ncnc(C2CC2)c1-c1ncc2c(n1)N(Cc1ccc(-n3nc(C(F)(F)F)cc3C)cc1)CCO2]

1-7 was synthesized in an analogous way as 1-2.

¹H NMR (400 MHz, DMSO-d6) δ 8.70-8.64 (m, 1H), 8.17 (d, J=8.0 Hz, 1H), 7.53 (q, J=8.4 Hz, 4H), 6.76 (s, 1H), 4.98 (d, J=3.6 Hz, 2H), 4.36 (s, 2H), 3.91-3.86 (m, 3H), 3.72 (s, 2H), 2.35-2.30 (m, 3H), 1.87 (s, 1H), 1.02 (s, 2H), 0.85 (dd, J=8.0, 3.4 Hz, 2H). LC-MS(ESI) m/z 524 [M+H]

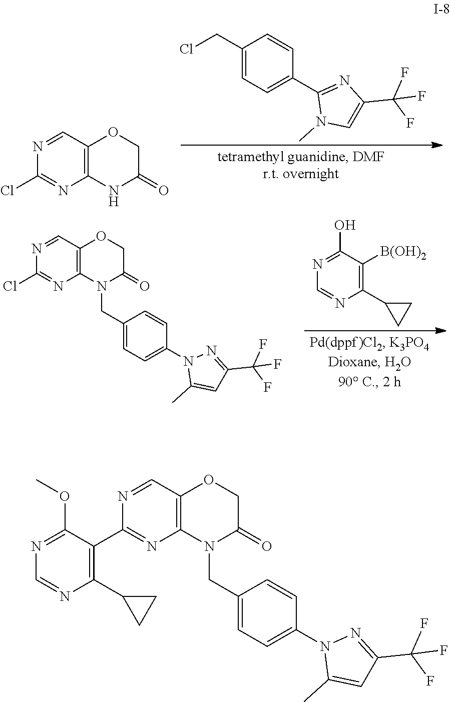 [SMILES: O=C1COc2cnc(Cl)nc2N1 ; Cn1cc(C(F)(F)F)nc1-c1ccc(CCl)cc1 ; Cc1cc(C(F)(F)F)nn1-c1ccc(CN2C(=O)COc3cnc(Cl)nc32)cc1 ; OB(O)c1c(O)ncnc1C1CC1 ; COc1ncnc(C2CC2)c1-c1ncc2c(n1)N(Cc1ccc(-n3nc(C(F)(F)F)cc3C)cc1)C(=O)CO2]

### Step 1: 2-Chloro-8-({4-[5-Methyl-3-(Trifluoromethyl) Pyrazol-1-Yl]Phenyl}Methyl)Pyrido[2,3-d]Pyrimidin-7-One

To a stirred solution of 2-chloro-8H-pyrido[2,3-d]pyrimidin-7-one (50 mg, 0.275 mmol, 1 equiv) and 1-[4-(chloromethyl)phenyl]-5-methyl-3-(trifluoromethyl)pyrazole (68.07 mg, mmol, 0.9 equiv) in DMF (1 mL) was added 1,1,3,3-tetramethylguanidine (47.57 mg, 0.413 mmol, 1.5 equiv) at room temperature. The resulting mixture was stirred overnight at room temperature under nitrogen atmosphere. The residue was purified by reverse flash chromatography (C18 silica gel column, eluting with 0% to 100% MeCN/water (0.1% TFA) in 10 min with UV detector (254 nm)) to afford 2-chloro-8-({4-[5-methyl-3-(trifluoromethyl) pyrazol-1-yl]phenyl}methyl)pyrido[2,3-d]pyrimidin-7-one (60 mg, 51.91%) as a white solid. LC-MS (ESI) m/z 420.7 [M+H].

### Step 2: 2-(4-Cyclopropyl-6-Methoxypyrimidin-5-Yl)-8-({4-[5-Methyl-3-(Trifluoromethyl)Pyrazol-1-Yl]Phenyl}Methyl)Pyrido[2,3-d]Pyrimidin-7-One

To a stirred solution of 2-chloro-8-({4-[5-methyl-3-(trifluoromethyl)pyrazol-1-yl]phenyl}methyl)pyrido[2,3-d]pyrimidin-7-one (50 mg, 0.119 mmol, 1 equiv) and 4-cyclopropyl-6-methoxypyrimidin-5-ylboronic acid (34.66 mg, 0.178 mmol, 1.5 equiv) in 1,4-dioxane (1 mL) and H₂O (0.2 mL) were added K₃PO₄ (63.21 mg, 0.297 mmol, 2.5 equiv) and Pd(dppf)C12 (17.43 mg, 0.024 mmol, 0.2 equiv) at room temperature. The resulting mixture was stirred for 2 h at 90° C. under nitrogen atmosphere. The resulting mixture was filtered, the filter cake was washed with 1,4-dioxane (3×5 mL). The filtrate was concentrated under reduced pressure. The crude product was purified by Prep-HPLC with the following conditions (Column: Xselect CSH C18 OBD Column 30*150 mm 51 μm, n; Mobile Phase A: Water(0.05% TFA), Mobile Phase B: ACN; Flow rate: 60 mL/min; Gradient: 55% B to 95% B in 7 min, 95% B; Wave Length: 254/220 nm; RT1(min): 6.02) to afford 2-(4-cyclopropyl-6-methoxypyrimidin-5-yl)-8-({4-[5-methyl-3-(trifluoromethyl)pyrazol-1-yl]phenyl}methyl)pyrido[2,3-d]pyrimidin-7-one (15.0 mg, 22.68%) as an off-white solid. ¹H NMR (300 MHz, DMSO-d6) δ 9.30 (s, 1H), 8.70 (s, 1H), 8.17 (d, J=9.5 Hz, 1H), 7.68-7.27 (m, 4H), 6.92 (d, J=9.5 Hz, 1H), 6.74 (s, 1H), 5.59 (s, 2H), 3.83 (s, 3H), 2.31 (d, J=0.7 Hz, 3H), 1.81-1.65 (m, 1H), 1.02 (d, J=4.2 Hz, 2H), 0.78 (dd, J=7.9, 3.3 Hz, 2H). LC-MS (ESI) m/z 534.1 [M+H].

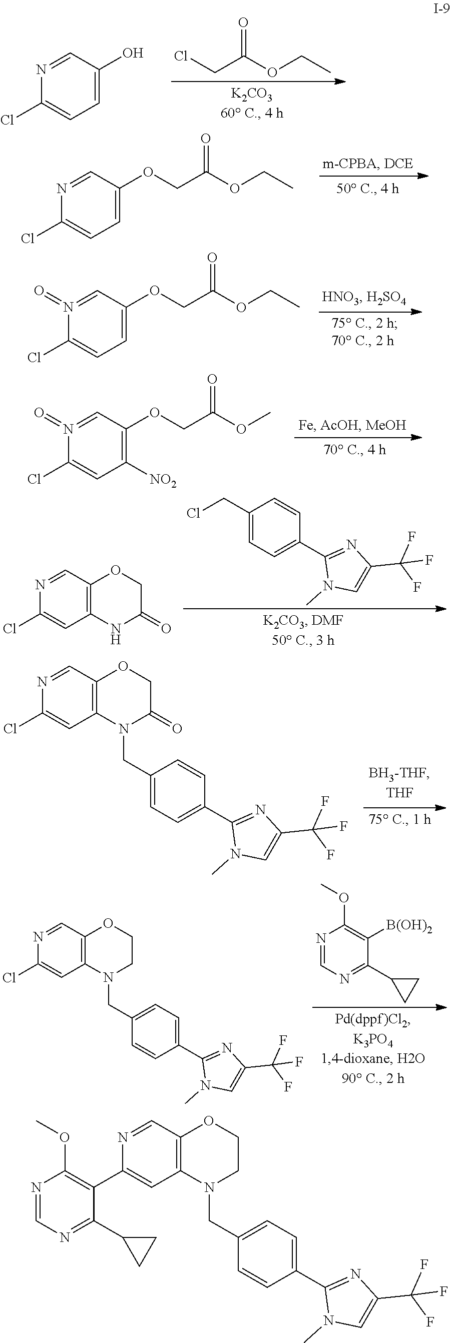

### Step 1: Ethyl 2-1(6-Chloropyridin-3-Yl)Oxy]Acetate

A solution of 6-chloropyridin-3-ol (7.7 g, 59.441 mmol, 1 equiv) in ACN (154.01 mL) was treated with potassium carbonate (23.17 g, 166.435 mmol, 2.8 equiv) and ethyl chloroacetate (9.11 g, 74.301 mmol, 1.25 equiv) for 4 h at 60° C. under nitrogen atmosphere. The mixture was allowed to cool down to room temperature. The resulting mixture was filtered, and concentrated under reduced pressure to afford ethyl 2-[(6-chloropyridin-3-yl)oxy]acetate (8 g, 62.42%) as a reddish brown solid. LC-MS (ESI) m/z 216.0 [M+H]

### Step 2: Ethyl 2-[(6-Chloro-1-Oxo-1Lambda5-Pyridin-3-Yl)Oxy]Acetate

A solution of ethyl 2-[(6-chloropyridin-3-yl)oxy]acetate (5 g, 23.188 mmol, 1 equiv) in DCE (100.00 mL, 1263.282 mmol, 54.48 equiv) was treated with m-CPBA (8.00 g, 46.376 mmol, 2.0 equiv) for 4 h at 50° C. under nitrogen atmosphere, and then stirred overnight at room temperature. The resulting mixture was concentrated under reduced pressure. The residue was purified by reverse flash chromatography (C18 silica gel column, eluting with 0% to 100% ACN/water (0.1% FA) in 10 min with UV detector (254 nm)). The resulting mixture was concentrated concentrated under reduced pressure and freeze-drie to afford ethyl 2-[(6-chloro-1-oxo-1lambda5-pyridin-3-yl)oxy]acetate (4 g, 74.47%) as a yellow solid. LC-MS (ESI) m/z 232.15 [M+H]

### Step 3 Methyl 2-[(6-Chloro-4-Nitro-1-Oxo-1Lambda5-Pyridin-3-Yl)Oxy]Acetate

A solution of ethyl 2-[(6-chloro-1-oxo-1lambda5-pyridin-3-yl)oxy]acetate (1000 mg, 4.317 mmol, 1 equiv) in H2504 (4 mL, 75.048 mmol, 17.38 equiv) was dropwise treated with HNO 3 (2 mL, 44.595 mmol, 10.33 equiv) for several minutes at 0° C. under nitrogen atmosphere. The reaction mixture was heated to 40° C. in an oil batch. The temperature was slowly raised to over 1 h and then maintained there for 2 h. The mixture was then cooled over ice, adjusted to pH 9 by the addition of 50% NaOH, dehydrated in vacuum, washed with 3×2 mL of MeOH, and concentrated under reduced pressure. The residue was dissolved in MeOH(50 ml) and treated with H2504 (1 mL) for 2 h at 70° C. under nitrogen atmosphere. The resulting mixture was concentrated under reduced pressure, diluted with water (20 ml), basified to pH 9 with NaOH, and extracted with EtOAc (3×15 mL). The combined organic layers were dried over anhydrous Na₂SO₄, filtered and concentrated under reduced pressure. The residue was purified by reverse flash chromatography (C18 silica gel column, eluting with 0% to 100% ACN/water (0.1% FA) in min with UV detector (254 nm)), concentrated under reduced pressure, and freeze-dried to afford methyl 2-[(6-chloro-4-nitro-1-oxo-1lambda5-pyridin-3-yl)oxy]acetate (200 mg, 17.64%) as a white solid. LC-MS (ESI) m/z 263.15 [M+H]

### Step 4: 7-Chloro-1H,311-Pyrido[3,4-b][1,4]Oxazin-2-One

A solution of methyl 2-[(6-chloro-4-nitro-1-oxo-1lambda5-pyri din-3-yl)oxy]acetate (210 mg, 0.800 mmol, 1 equiv) in MeOH (6 mL) was treated with Fe (312.61 mg, mmol, 7 equiv) and AcOH (0.25 mg, 0.004 mmol, 0.01 equiv) for 4 h at 70° C. under nitrogen atmosphere. The hot solution was filtered through a pad of celite and concentrated. The residue was purified by reverse flash chromatography (C18 silica gel column, eluting with 0% to 100% ACN/water (0.1% NH₃—H₂O) in 10 min with UV detector (254 nm)) to yield 7-chloro-1H,3H-pyrido[3,4-b][1,4]oxazin-2-one (140 mg, 94.85%) as a reddish brown solid. LC-MS (ESI) m/z 185.00 [M+H]

### Step 5 7-Chloro-1-({4-[1-Methyl-4-(Trifluoromethyl)Imidazol-2-Yl]Phenyl}Methyl)-311-Pyrido[3,4-b][1,4]Oxazin-2-One

A solution of 7-chloro-1H,3H-pyrido[3,4-b][1,4]oxazin-2-one (200 mg, 1.084 mmol, 1 equiv) in DMF (10.00 mL) was treated with K₂CO₃ (449.25 mg, 3.252 mmol, 3 equiv) and 2-[4-(bromomethyl)phenyl]-1-methyl-4-(trifluoromethyl)imidazole (449.52 mg, 1.409 mmol, 1.3 equiv) for 3h at 50° C. under nitrogen atmosphere. The residue was purified by reverse flash chromatography (C18 silica gel column, eluting with 0% to 100% ACN/water (0.1% NH₃—H₂O) in 10 min with UV detector (254 nm)) to afford 7-chloro-1-({4-[1-methyl-4-(trifluoromethyl)imidazol-2-yl]phenyl methyl)-3H-pyrido[3,4-b][1,4]oxazin-2-one (250 mg, 54.57%) as an off-white solid. LC-MS (ESI) m/z 423.2 [M+H]

### Step 6. 2-[4-({7-Chloro-211,311-Pyrido[3,4-b][1,4]Oxazin-1-Yl}Methyl)Phenyl]-1-Methyl-4-(Trifluoromethyl)Imidazole

A mixture of 7-chloro-1-({4-[1-methyl-4-(trifluoromethyl)imidazo]-2-yl]phenyl}methyl)-3H-pyrido[3,4-b][1,4]oxazin-2-one (100 mg, 0.237 mmol, 1 equiv) and BH₃-THF (0.59 mL, 0.593 mmol, 2.5 equiv,. 1 M in THF) in THF (1.5 mL) was stirred for 1 h at 75° C. under nitrogen atmosphere. The reaction was quenched with MeOH at room temperature. The resulting mixture was concentrated under reduced pressure. The residue was purified by reverse flash chromatography (C18 silica gel column, eluting with 0% to 100% ACN/water (0.1% FA) in min with UV detector (254 nm)) and freeze-dried to afford 2-[4-({7-chloro-2H,3H-pyrido[3,4-b][1,4]oxazin-1-yl}methyl)phenyl]-1-methyl-4-(trifluoromethyl)imidazole (60 mg, 62.05%) as an off-white solid. LC-MS (ESI) m/z 409.1 [M+H]

### Step 7. 4-Cyclopropyl-6-Methoxy-5-[1-({4-[1-Methyl-4-(Trifluoromethyl)Imidazo]-2-Yl]Phenyl}Methyl)-211,311-Pyrido[3,4-b][1,4]Oxazin-7-Yl]Pyrimidine

A solution of 2-[4-({7-chloro-2H,3H-pyrido[3,4-b][1,4]oxazin-1-yl}methyl)phenyl]-1-methyl-4-(trifluoromethyl)imidazole (60 mg, 0.147 mmol, 1 equiv) and 4-cyclopropyl-6-methoxypyrimidin-5-ylboronic acid (42.71 mg, 0.220 mmol, 1.5 equiv) in 1,4-dioxane (1.8 mL) and H₂O (0.2 mL) was treated with K₃PO₄ (77.88 mg, 0.367 mmol, 2.5 equiv) and Pd(dppf)C12 (32.22 mg, 0.044 mmol, 0.3 equiv) for 2 h at 90° C. under nitrogen atmosphere. The residue was purified by reverse flash chromatography (C18 silica gel column, eluting with 0% to 100% ACN/water (0.1% NH₃—H₂O) in 10 min with UV detector (254 nm)). The crude product was purified by Prep-HPLC with the following conditions (Column: Sunfire prep C18 column, mm, 5 μm; Mobile Phase A: Water(0.1% FA), Mobile Phase B: ACN; Flow rate: 60 mL/min; Gradient: 15% B to 45% B in 7 min, 45% B; Wave Length: 254/220 nm; RT1(min): 4.45) to afford 4-cyclopropyl-6-methoxy-5-[1-({4-[1-methyl-4-(trifluoromethyl)imidazo]-2-yl]phenyl}methyl)-2H,3H-pyrido[3,4-b][1,4]oxazin-7-yl]pyrimidine (10.9 mg, 14.21%) as an off-white solid. ¹H-NMR (400 MHz, DMSO-d6): ¹H NMR (400 MHz, DMSO-d6) δ 8.54 (s, 1H), 7.92 (d, J=4.5 Hz, 2H), 7.70 (d, J=7.9 Hz, 2H), 7.42 (d, J=7.9 Hz, 2H), 6.80 (s, 1H), 4.68 (s, 2H), 4.31 (t, J=4.3 Hz, 2H), 3.75 (d, J=11.0 Hz, 6H), 3.57 (t, J=4.4 Hz, 2H), 1.78 (dd, J=8.2, 3.9 Hz, 1H), 0.93 (p, J=3.7 Hz, 2H), 0.77 (dq, J=7.1, 3.4 Hz, 2H). LC-MS (ESI) m/z 523.25 [M+H]

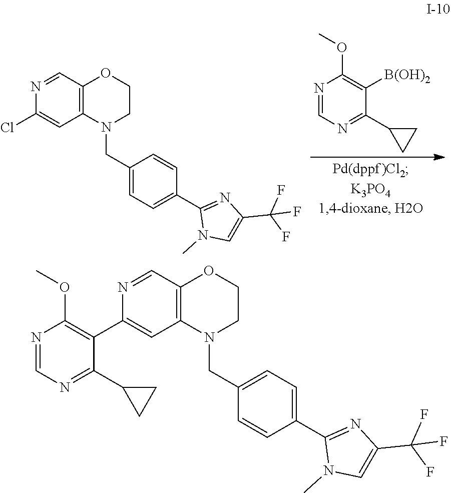

A solution of 7-chloro-1-({4-[1-methyl-4-(trifluoromethyl)imidazol-2-yl]phenyl}methyl)-3H-pyrido[3,4-b][1,4]oxazin-2-one (80 mg, 0.189 mmol, 1 equiv) and 4-cyclopropyl-6-methoxypyrimidin-5-ylboronic acid (55.06 mg, 0.283 mmol, 1.5 equiv) in 1,4-dioxane (1.8 mL) and H₂O (0.16 mL, 8.872 mmol, 46.94 equiv) was treated with K₃PO₄ (100.41 mg, 0.473 mmol, 2.5 equiv) and Pd(dppf)Cl₂ (27.69 mg, 0.038 mmol, 0.2 equiv) for 2 h at 90° C. under nitrogen atmosphere. The resulting mixture was filtered, and the filter cake was washed with 1,4-dioxane (3×10 mL). The filtrate was concentrated under reduced pressure. The residue was purified by Prep-HPLC with the following conditions (Column: XBridge Prep OBD C18 Column, mm, 51 μm; Mobile Phase A: water (0.05%NH₃—H₂O), Mobile Phase B: ACN; Flow rate: 60 mL/min; Gradient: 28% B to 60% B in 8 min, 60% B; Wave Length: 220 nm; RT1(min): 7.35) to afford 7-(4-cyclopropyl-6-methoxypyrimidin-5-yl)-1-({4-[1-methyl-4-(trifluoromethyl)imi dazol-2-yl]phenyl methyl)-3H-pyrido[3,4-b][1,4]oxazin-2-one (20.4 mg, 20.05%) as an off-white solid. ¹H-NMR(400 MHz, DMSO-d6): δ 8.59 (s, 1H), 8.35 (s, 1H), 7.92 (d, J=1.3 Hz, 1H), 7.73-7.66 (m, 2H), 7.45 (d, J=8.1 Hz, 2H), 7.29 (s, 1H), 5.26 (s, 2H), 5.01 (s, 2H), 3.76 (s, 3H), 3.69 (s, 3H), 1.71 (tt, J=8.3, 4.6 Hz, 1H), 0.95 (p, J=3.7 Hz, 2H), 0.76 (dq, J=6.9, 3.5 Hz, 2H). LC-MS (ESI) m/z 537.2 [M+H].

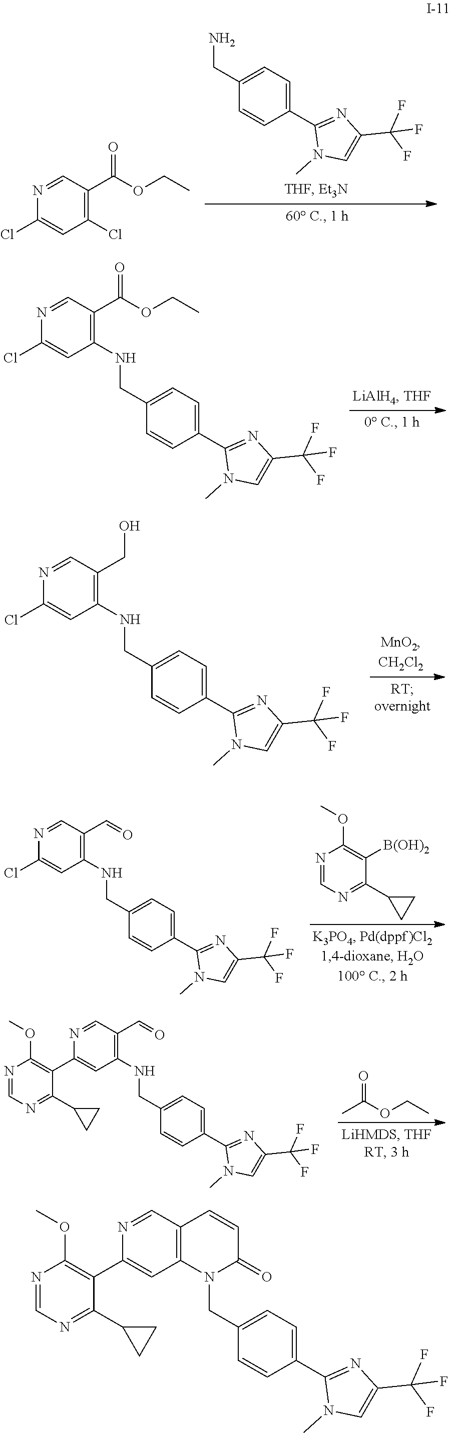

### Step 1: 1-{6-Chloro-4-1({4-[1-Methyl-4-(Trifluoromethyl)Imidazol-2-Yl]Phenyl}Methyl)Amino]Pyridin-3-Yl}-2-Methoxyethanone

To a stirred solution of ethyl 4,6-dichloropyridine-3-carboxylate (100 mg, 0.454 mmol, 1 equiv) and 1-{4-[1-methyl-4-(trifluoromethyl)imidazo]-2-yl]phenyl methanamine (127.59 mg, 0.499 mmol, 1.1 equiv) in tetrahydrofuran (2 mL, 20 equiv) was added triethylamine (91.7 mg, 0.908 mmol, 2 equiv) at room temperature under nitrogen atmosphere. The resulting mixture was stirred for 1 h at 60° C. under nitrogen atmosphere. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford 1-{6-chloro-4-[({4-[1-methyl-4-(trifluoromethyl)imidazol-2-yl]phenyl methyl)amino]pyridin-3-yl}-2-methoxyethanone (78 mg, 39.11%) as a light yellow solid. LC-MS (ESI) m/z 439.1 [M+H]

### Step 2. {6-Chloro-4-1({4-[1-Methyl-4-(Trifluoromethyl)Imidazol-2-Yl]Phenyl}Methyl)Amino]Pyridin-3-Yl}Methanol

To a stirred 1-{6-chloro-44({4-[1-methyl-4-(trifluoromethyl)imidazo]-2-yl]phenyl methyl)amino]pyridin-3-yl}-2-methoxyethanone (78 mg, 0.178 mmol, 1 equiv) in tetrahydrofuran (1.5 mL, 20 equiv) was added LiAlH₄ (20.24 mg, 0.534 mmol, 3 equiv) in portions at 0° C. under nitrogen atmosphere. The resulting mixture was stirred for 1 h at under nitrogen atmosphere. The reaction was quenched with 10H2O.Na₂SO₄(100 mg) at resulting mixture was filtered, the filter cake was washed with THF (3×5 mL). The filtrate was concentrated under reduced pressure. The residue was dissolved in THF (2 mL). The residue was purified by reverse flash chromatography (C18 silica gel column, eluting with 10% to 50% MeCN/water (0.1% FA) in 10 min with UV detector (254 nm)) to afford {6-chloro-4-[({4-[1-methyl-4-(trifluoromethyl)imidazo]-2-yl]phenyl}methyl)amino]pyri din-3-yl}methanol (23.5 mg, 33.32%) as a white solid. LC-MS (ESI) m/z 397.1 [M+H]

### Step 3. 6-Chloro-4-1({4-[1-Methyl-4-(Trifluoromethyl)Imidazol-2-Yl]Phenyl}Methyl)Amino]Pyridine-3-Carbaldehyde

To a stirred solution of {6-chloro-44({4-[1-methyl-4-(trifluoromethyl)imidazo]-2-yl]phenyl}methyl)amino]pyridin-3-yl}methanol (85 mg, 0.214 mmol, 1 equiv) in methylene chloride (4 mL, 20 equiv) was added manganese dioxide (55.87 mg, 0.642 mmol, 3.0 equiv) at room temperature under nitrogen atmosphere. The reaction mixture was kept at room temperature overnight. The resulting mixture was filtered, the filter cake was washed with CH₂Cl₂ (3×5 mL). The filtrate was concentrated under reduced pressure. The residue was dissolved in MeOH (2 mL). The residue was purified by reverse flash chromatography (C18 silica gel column, eluting with 0% to 100% MeCN/water (0.1% FA) in 10 min with UV detector (254 nm)) to afford 6-chloro-44({4-[1-methyl-4-(trifluoromethyl)imidazol-2-yl]phenyl}methyl)amino]pyri dine-3-carbaldehyde (60 mg, 70.95%) as a light yellow solid. LC-MS (ESI) m/z 395.0 [M+H].

### Step 4. 6-(4-Cyclopropyl-6-Methoxypyrimidin-5-Yl)-4-1({4-[1-Methyl-4-(Trifluoromethyl)Imidazol-2-Yl]Phenyl}Methyl)Amino]Pyridine-3-Carbaldehyde

To a stirred mixture of 6-chloro-4-[({4-[1-methyl-4-(trifluoromethyl)imidazol-2-yl]phenyl}methyl)amino]pyridine-3-carbaldehyde (20 mg, 0.051 mmol, 1 equiv) and 4-cyclopropyl-6-methoxypyrimidin-5-ylboronic acid (14.74 mg, 0.076 mmol, 1.5 equiv) in 1,4-dioxane (0.9 mL, 1.020 mmol, 20 equiv) and water (0.1 mL, 1.020 mmol, 20 equiv) were added K₃PO₄ (32.26 mg, 0.153 mmol, 3 equiv) and Pd(dppf)C12 (7.41 mg, 0.010 mmol, 0.2 equiv) at room temperature under nitrogen atmosphere. The resulting mixture was stirred for 2 h at 100° C. under nitrogen atmosphere. The resulting mixture was concentrated under reduced pressure. The residue was dissolved in THF (1 mL). The residue was purified by reverse flash chromatography (C18 silica gel column, eluting with 0% to 100% MeCN/water (0.1% FA) in 10 min with UV detector (254 nm)) to afford 6-(4-cyclopropyl-6-methoxypyrimidin-5-yl)-44({4-[1-methyl-4-(trifluoromethyl)imidazo]-2-yl]phenyl}methyl)amino]pyri dine-3-carb aldehyde (15 mg, 58.23%) as a yellow solid.LC-MS (ESI) m/z 509.1 [M+H]

### Step 5: 7-(4-Cyclopropyl-6-Methoxypyrimidin-5-Yl)-1-({4-[1-Methyl-4-(Trifluoromethyl)Imidazol-2-Yl]Phenyl}Methyl)-1,6-Naphthyridin-2-One

To a stirred solution of ethyl acetate (34.65 mg, 0.392 mmol, 4.0 equiv) in THF (2 mL, 20 equiv) was added LiHMDS (0.392 mL, 0.392 mmol, 4.0 equiv., 1 M in THF) dropwise at −78° C. under nitrogen atmosphere. The resulting mixture was stirred for 10 min at −78° C. under nitrogen atmosphere. To the above mixture was added 6-(4-cyclopropyl-6-methoxypyrimidin-5-yl)-44 {4-[1-methyl-4-(trifluoromethyl)imidazo]-2-yl]phenyl}methyl)amino]pyridine-3-carbaldehyde (50 mg, 0.098 mmol, 1 equiv) dropwise at −78° C. The resulting mixture was stirred for additional 3 h at room temperature. The reaction was quenched with water at 0° C. The resulting mixture was extracted with EtOAc (3×5 mL). The combined organic layers were washed with brine (2×2 mL), dried over anhydrous Na₂SO₄, filtered and concentrated under reduced pressure. The residue was purified by Prep-HPLC (XBridge Shield RP18 OBD Column, 30*150 mm, 51 μm; Mobile Phase A: Water(0.1% FA), Mobile Phase B: ACN; Flow rate: 60 mL/min; Gradient: 40% B to 75% B in 7 min, 75% B; Wave Length: 254/220 nm; RT1(min): 3.45;) to afford 7-(4-cyclopropyl-6-methoxypyrimidin-5-yl)-1-({4-[1-methyl-4-(trifluoromethyl)imidazo]-2-yl]phenyl}methyl)-1,6-naphthyridin-2-one (21.1 mg, 40.26%) as a white solid. ¹H NMR (300 MHz, DMSO-d6) δ 9.03 (s, 1H), 8.64 (s, 1H), 8.17 (d, J=9.6 Hz, 1H), 7.92 (d, J=1.4 Hz, 1H), 7.73-7.61 (m, 3H), 7.37 (d, J=8.1 Hz, 2H), 6.88 (d, J=9.5 Hz, 1H), 5.58 (s, 2H), 3.75 (s, 3H), 3.69 (s, 3H), 1.74-1.64 (m, 1H), 0.97 (d, J=4.4 Hz, 2H), 0.75 (dd, J=8.1, 3.3 Hz, 2H). LC-MS (ESI) m/z 533.10 [M+H]

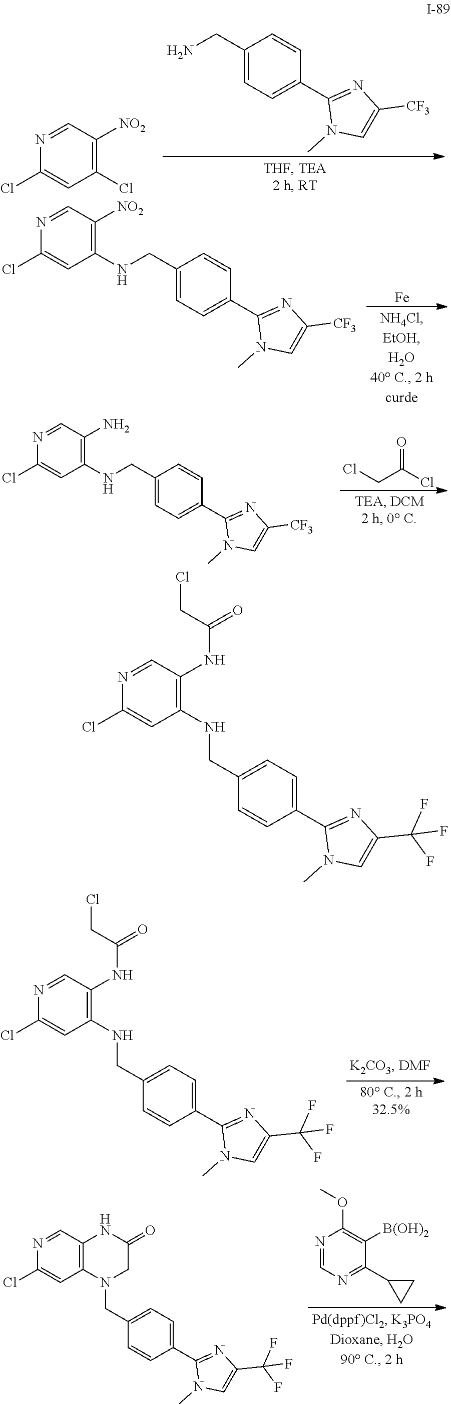

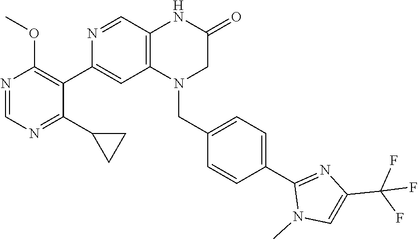 [SMILES: COc1ncnc(C2CC2)c1-c1cc2c(cn1)NC(=O)CN2Cc1ccc(-c2nc(C(F)(F)F)cn2C)cc1]

### Step 1: 2-Chloro-N-({4-[1-Methyl-4-(Trifluoromethyl)Imidazol-2-Yl]Phenyl}Methyl)-5-Nitropyridin-4-Amine

A mixture of 2,4-dichloro-5-nitropyridine (200 mg, 1.036 mmol, 1 equiv), 1-{4-[1-methyl-4-(trifluoromethyl)imidazo]-2-yl]phenyl}methanamine (310 mg, 1.215 mmol, 1.17 equiv) and TEA(65 mg, 1.5 mmol, 1.2 equiv) in THF (10 mL) was stirred for 2 h at room temperature under nitrogen atmosphere. The resulting mixture was concentrated under reduced pressure. The residue was purified by reverse flash chromatography (C18 silica gel column, eluting with 0% to 100% MeCN/water(0.1% FA) in 10 min with UV detector(254 nm)) to afford 2-chloro-N-({4-[1-methyl-4-(trifluoromethyl)imidazol-2-yl]phenyl methyl)-5-nitropyridin-4-amine (260 mg, 60.93%) as a colorless oil. LC-MS (ESI) m/z 412 [M+H].

### Step 2: 6-Chloro-N4-({4-[1-Methyl-4-(Trifluoromethyl)Imidazol-2-Yl]Phenyl}Methyl)Pyridine-3,4-Diamine

A solution of 2-chloro-N-({4-[1-methyl-4-(trifluoromethyl)imidazo]-2-yl]phenyl}methyl)-5-nitropyridin-4-amine (260 mg, 0.631 mmol, 1 equiv), iron (280 mg, 5.014 mmol, 7.94 equiv), NH 4 C1 (180 mg, 3.365 mmol, 5.33 equiv) and H₂O (2 mL, 111.019 mmol, 175.82 equiv) in EtOH (8 mL) was stirred for 2 h at 40° C. under nitrogen atmosphere. The resulting mixture was filtered, the filter cake was washed with EtOH (2×100 mL). The filtrate was concentrated under reduced pressure to afford 6-chloro-N4-({4-[1-methyl-4-(trifluoromethyl)imidazo]-2-yl]phenyl}methyl)pyridine-3,4-diamine (350 mg, 145.19%) as a brown solid. LC-MS (ESI) m/z 382 [M+H].

### Step 3: N-(5-Amino-2-Chloropyridin-4-Yl)-2-Chloro-N-({4-[1-Methyl-4-(Trifluoromethyl)Imidazol-2-Yl]Phenyl}Methyl)Acetamide

To a solution of 6-chloro-N4-({4-[1-methyl-4-(trifluoromethyl)imidazol-2-yl]phenyl methyl)pyri dine-3,4-di amine (450 mg, 1.179 mmol, 1 equiv) in DCM (10 mL) was added TEA (430 mg, 4.249 mmol, 3.61 equiv) at room temperature under nitrogen atmosphere followed by the addition of chloroacetyl chloride (170 mg, 1.505 mmol, 1.28 equiv) dropwise at 0° C. The resulting mixture was stirred for 2 h at room temperature under nitrogen atmosphere. The resulting mixture was concentrated under reduced pressure. The residue was purified by reverse flash chromatography (C18 silica gel column, eluting with 0% to 100% MeCN/water(0.1% FA) in 10 min with UV detector (254 nm)) to afford N-(5-amino-2-chloropyridin-4-yl)-2-chloro-N-({4-[1-methyl-4-(trifluoromethyl)imidazol-2-yl]phenyl methyl)acetamide (150 mg, 27.77%) as a colorless oil. LC-MS (ESI) m/z 458 [M+H].

### Step 4: 7-Chloro-1-({4-[1-Methyl-4-(Trifluoromethyl)Imidazol-2-Yl]Phenyl}Methyl)-211,411-Pyrido[3,4-b]Pyrazin-3-One

A solution of 2-chloro-N-{6-chloro-4-[({4-[1-methyl-4-(trifluoromethyl)imidazol-2-yl]phenyl}methyl)amino]pyridin-3-yl}acetamide (100 mg, 0.218 mmol, 1 equiv) and K₂CO₃ (60 mg, 0.434 mmol, 1.99 equiv) in DMF (5 mL) was stirred for 2 h at under nitrogen atmosphere. The resulting mixture was concentrated under reduced pressure. The residue was purified by reverse flash chromatography (C18 silica gel column, eluting with 0% to 100% MeCN/water(0.1% FA) in 10 min with UV detector(254 nm) to afford 7-chloro-1-({4-[1-methyl-4-(trifluoromethyl)imidazo]-2-yl]phenyl}methyl)-2H,4H-pyrido[3,4-b]pyrazin-3-one (30 mg, 32.59%) as an off-white solid. LC-MS (ESI) m/z 422 [M+H].

### Step 5. Synthesis of 4-Cyclopropyl-5-[1-({4-[1-Ethyl-4-(Trifluoromethyl)Imidazol-2-Yl]Phenyl}Methyl)Pyrazolo[4,3-c]Pyridin-6-Yl]-6-Methoxypyrimidine

A solution of 2444 {6-chloropyrazolo[4,3-c]pyridin-1-yl}methyl)phenyl]-1-ethyl-4-(trifluoromethyl)imidazole (70 mg, 0.172 mmol, 1 equiv), 4-cyclopropyl-6-methoxypyrimidin-acid (38 mg, 0.196 mmol, 1.14 equiv), Pd(dppf)Cl₂ (20 mg, 0.027 mmol, 0.16 equiv) in H₂O (1 mL) and dioxane (4 mL) was stirred for 2 h at 90° C. under nitrogen atmosphere. The resulting mixture was concentrated under reduced pressure. The residue was purified by reverse flash chromatography (C18 silica gel column, eluting with 0% to 100% MeCN/water(0.1% FA) in 10 min with UV detector(254 nm) to afford 4-cyclopropyl-5-[1-({4-[1-ethyl-4-(trifluoromethyl)imidazo]-2-yl]phenyl methyl)pyrazolo[4,3-c]pyridin-6-yl]-6-methoxypyrimidine (8 mg, 8.63%) as an off-white solid ¹H-NMR: ¹H NMR (400 MHz, DMSO-d6) δ 10.75 (s, 1H), 8.55 (s, 1H), 7.94 (d, J=8.8 Hz, 2H), 7.70 (d, J=8.0 Hz, 2H), 7.45 (d, J=7.9 Hz, 2H), 6.82 (s, 1H), 4.63 (s, 2H), 4.05 (s, 2H), 3.76 (d, J=10.6 Hz, 6H), 1.80 (s, 1H), 1.24 (s, 1H), 0.94 (s, 2H), 0.79 (d, J=7.7 Hz, 2H). LC-MS (ESI) m/z 536.15 [M+H].

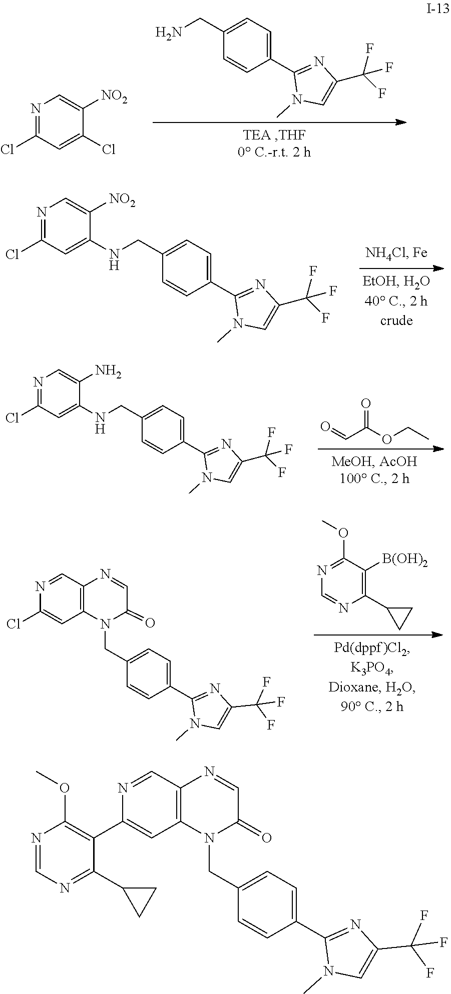

### Step 1: 2-Chloro-N-({4-[1-Methyl-4-(Trifluoromethyl)Imidazol-2-Yl]Phenyl}Methyl)-5-Nitropyridin-4-Amine

To a stirred solution of 2,4-dichloro-5-nitropyridine (415.83 mg, 2.155 mmol, 1.1 equiv) and TEA (396.46 mg, 3.918 mmol, 2 equiv) in THF (4 mL) was added 1-{4-[1-methyl-4-(trifluoromethyl)imidazo]-2-yl]phenyl}methanamine (500 mg, 1.959 mmol, 1.00 equiv) in THF(1 mL) dropwise at 0° C. The resulting mixture was stirred for 2 h at room temperature and concentrated under reduced pressure. The residue was purified by reverse flash chromatography (C18 silica gel column, eluting with 10% to 100% MeCN/water (0.1% FA) in 10 min with UV detector (254 nm)) to afford 2-chloro-N-({4-[1-methyl-4-(trifluoromethyl)imidazol-2-yl]phenyl}methyl)-5-nitropyridin-4-amine (400 mg, 49.5%) as a yellow solid. LC-MS (ESI) m/z 412.7 [M+H].

### Step 2: 6-Chloro-N4-({4-[1-Methyl-4-(Trifluoromethyl)Imidazol-2-Yl]Phenyl}Methyl)Pyridine-3,4-Diamine

To a stirred solution of 2-chloro-N-({4-[1-methyl-4-(trifluoromethyl)imidazol-2-yl]phenyl}methyl)-5-nitropyridin-4-amine (150 mg, 0.364 mmol, 1 equiv) and NH 4 C1 (97.43 mg, 1.820 mmol, 5 equiv) in ethanol (2.5 mL) and water (0.6 mL) was added Fe (122.06 mg, 2.184 mmol, 6 equiv) at room temperature. The resulting mixture was stirred for 2 h at 40° C. The resulting mixture was filtered, the filter cake was washed with ethanol (3×10 mL). The filtrate was concentrated under reduced pressure. The crude product was used in the next step directly without further purification. LC-MS (ESI) m/z 382.7 [M+H].

### Step 3: 7-Chloro-1-({4-[1-Methyl-4-(Trifluoromethyl)Imidazol-2-Yl]Phenyl}Methyl)Pyrido[3,4-13]Pyrazin-2-One

To a stirred solution of 6-chloro-N4-({4-[1-methyl-4-(trifluoromethyl)imidazo]-2-yl]phenyl}methyl)pyridine-3,4-diamine (130 mg, 0.341 mmol, 1 equiv) and ethyl glyoxylate (104.28 mg, 1.023 mmol, 3 equiv) in MeOH (2 mL) was added AcOH (0.1 mL) dropwise at room temperature. The resulting mixture was stirred for 2 h at 100° C. and concentrated under reduced pressure. The residue was purified by reverse flash chromatography (C18 silica gel column, eluting with 10% to 100% MeCN/water (0.1% FA) in 10 min with UV detector (254 nm)) to afford 7-chloro-14 {4-[1-methyl-4-(trifluoromethyl)imidazo]-2-yl]phenyl methyl)pyrido[3,4-b]pyrazin-2-one (60 mg, 41.9%) as a brown yellow solid. LC-MS (ESI) m/z 420.7 [M+H].

### Step 4: 7-(4-Cyclopropyl-6-Methoxypyrimidin-5-Yl)-1-({4-[1-Methyl-4-(Trifluoromethyl)Imidazol-2-Yl]Phenyl}Methyl)Pyrido[3,4-13]Pyrazin-2-One

To a stirred solution of 7-chloro-1-({4-[1-methyl-4-(trifluoromethyl)imidazol-2-yl]phenyl}methyl)pyrido[3,4-b]pyrazin-2-one (60 mg, 0.143 mmol, 1 equiv) and 4-cyclopropyl-6-methoxypyrimidin-5-ylboronic acid (41.59 mg, 0.214 mmol, 1.5 equiv) in 1,4-dioxane (2 mL) and H₂O (0.4 mL) was added Pd(dppf)C12 (20.92 mg, 0.029 mmol, 0.2 equiv) and K₃PO₄ (75.85 mg, 0.357 mmol, 2.50 equiv) at room temperature. The resulting mixture was stirred for 2 h at 90° C. The resulting mixture was filtered, the filter cake was washed with 1,4-dioxane (2×10 mL). The filtrate was concentrated under reduced pressure. The crude product was purified by reverse flash chromatography (C18 silica gel column, eluting with 0% to 100% MeCN/water (0.1% FA) in min with UV detector (254 nm)) to afford 7-(4-cyclopropyl-6-methoxypyrimidin-5-yl)-1-({4-[1-methyl-4-(trifluoromethyl)imidazol-2-yl]phenyl}methyl)pyrido[3,4-b]pyrazin-2-one (7.7 mg, 9.88%) as a brown yellow solid. ¹H NMR (300 MHz, DMSO-d6) δ 9.12 (s, 1H), 8.65 (s, 1H), 8.46 (s, 1H), 7.93 (s, 1H), 7.75 (s, 1H), 7.69 (d, J=8.1 Hz, 2H), 7.47 (d, J=8.1 Hz, 2H), 5.52 (s, 2H), 3.73 (d, J=12.9 Hz, 6H), 1.81-1.62 (m, 1H), 0.99 (d, J=4.1 Hz, 2H), 0.78 (dd, J=8.1, 3.3 Hz, 2H). LC-MS (ESI) m/z 534.2 [M+H].

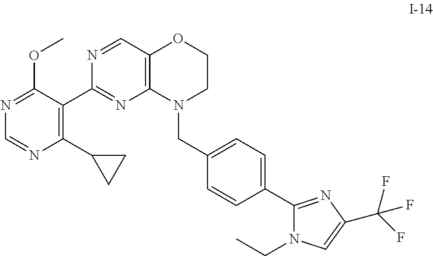 [SMILES: CCn1cc(C(F)(F)F)nc1-c1ccc(CN2CCOc3cnc(-c4c(OC)ncnc4C4CC4)nc32)cc1]

1-14 was made in an analogous way of 1-2 ¹H NMR (400 MHz, DMSO-d6) δ 8.67 (d, J=2.0 Hz, 1H), 8.18 (s, 1H), 8.03 (d, J=1.5 Hz, 1H), 7.61 (d, J=8.1 Hz, 2H), 7.47 (d, J=8.0 Hz, 2H), 4.97 (s, 2H), 4.36 (s, 2H), 4.07 (q, J=7.3 Hz, 2H), 3.89 (s, 3H), 3.71 (s, 2H), 1.87 (d, J=7.3 Hz, 1H), 1.32 (t, J=7.2 Hz, 3H), 1.01 (d, J=4.4 Hz, 2H), 0.84 (dt, J=8.0, 3.4 Hz, 2H). LC-MS(ESI) m/z 538 [M+H].

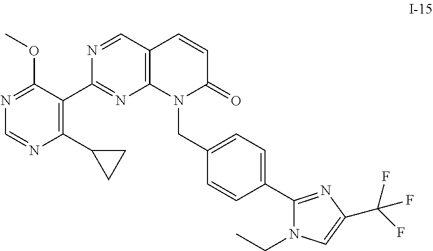 [SMILES: CCn1cc(C(F)(F)F)nc1-c1ccc(Cn2c(=O)ccc3cnc(-c4c(OC)ncnc4C4CC4)nc32)cc1]

1-15 was made in an analogous way as 1-4. ¹H NMR (400 MHz, DMSO-d6) δ 9.29 (s, 1H), 8.69 (s, 1H), 8.16 (d, J=9.5 Hz, 1H), 8.00 (d, J=1.3 Hz, 1H), 7.57-7.50 (m, 2H), 7.40 (d, J=8.3 Hz, 2H), 6.91 (d, J=9.5 Hz, 1H), 5.57 (s, 2H), 4.04 (q, J=7.2 Hz, 2H), 3.82 (s, 3H), 1.72 (m, 1H), 1.29 (t, J=7.2 Hz, 3H), 1.01 (m, 2H), 0.76 (m, 2H). LC-MS (ESI) m/z: 548.0 [M+H]

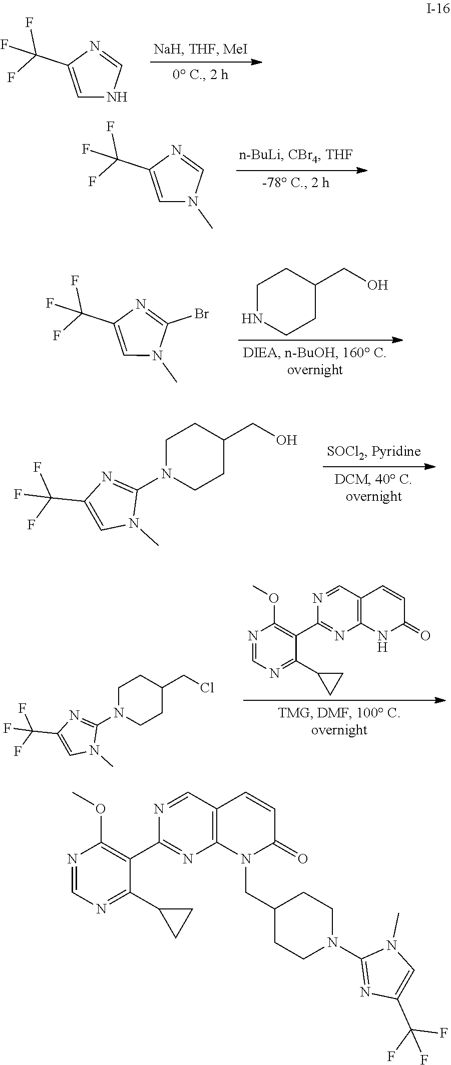

### Step 1: 1-Methyl-4-(Trifluoromethyl)Imidazole

A solution of 4-(trifluoromethyl)-1H-imidazole (3 g, 22.046 mmol, 1 equiv) in THF(30 mL) was treated with NaH (0.79 g, 33.069 mmol, 1.5 equiv) for 30 min at under nitrogen atmosphere followed by the addition of Mel (3.13 g, 22.046 mmol, 1 equiv) dropwise at 0° C. The resulting mixture was stirred for 2 h at room temperature under nitrogen atmosphere. The mixture was quenched with saturated NH₄Cl (aq.) (10 mL). The resulting mixture was extracted with EtOAc (3×100 mL). The combined organic layers were washed with water (2×50 mL), dried over anhydrous Na₂SO₄, filtered and concentrated under reduced pressure. The residue was purified by reverse flash chromatography (C18 silica gel column, eluting with 0% to 100% MeCN/water(0.1% FA) in 10 min with UV detector (254 nm)) to afford 1-methyl-4-(trifluoromethyl)imidazole (1.5 g, 45.33%) as a colorless oil LC-MS (ESI) m/z 150 [M+H].

### Step 2: 2-Bromo-1-Methyl-4-(Trifluoromethyl)Imidazole

A solution of 1-methyl-4-(trifluoromethyl)imidazole (100 mg, 0.666 mmol, 1 equiv) in THF(10 mL) was treated with n-BuLi (76.82 mg, 1.199 mmol, 1.8 equiv) for 30 min at −78° C. under nitrogen atmosphere followed by the addition of CBr₄ (441.86 mg, 1.332 mmol, 2 equiv) dropwise at −78° C. The resulting mixture was stirred for 2 h at room temperature under nitrogen atmosphere. The mixture was acidified to pH 5 with 1M HCl (5 mL). The resulting mixture was concentrated under reduced pressure. The residue was purified by reverse flash chromatography (C18 silica gel column, eluting with 0% to 100% MeCN/water(0.1% FA) in 10 min with UV detector (254 nm)) to afford 2-bromo-1-methyl-4-(trifluoromethyl)imidazole (50 mg, 32.77%) as a colorless oil. LC-MS (ESI) m/z 229 [M+H].

### Step 3: {1-[1-Methyl-4-(Trifluoromethyl)Imidazol-2-Yl]Piperidin-4-Yl}Methanol

To a stirred solution of 2-bromo-1-methyl-4-(trifluoromethyl)imidazole (50 mg, mmol, 1 equiv) and piperidin-4-ylmethanol (80.37 mg, 0.699 mmol, 3 equiv) in n-BuOH (2 mL, 21.880 mmol, 94.07 equiv) was added DIEA (90.18 mg, 0.699 mmol, 3 equiv) at room temperature under nitrogen atmosphere. The resulting mixture was stirred for overnight at 160° C. under nitrogen atmosphere. The residue was purified by reverse flash chromatography (C18 silica gel column, eluting with 0% to 100% MeCN/water(0.1% FA) in 10 min with UV detector (254 nm)) to afford {1-[1-methyl-4-(trifluoromethyl)imidazo]-2-yl]piperidin-4-yl}methanol (50 mg, 81.66%) as a yellow oil. LC-MS (ESI) m/z 263 [M+H].

### Step 4: 4-(Chloromethyl)-1-[1-Methyl-4-(Trifluoromethyl)Imidazol-2-Yl]Piperidine

To a stirred solution of {1-[1-methyl-4-(trifluoromethyl)imidazo]-2-yl]piperidin-4-yl}methanol (180 mg, 0.684 mmol, 1 equiv), Pyridine (0.1 mL, 0.001 mmol, 0.01 equiv) in DCM (3 mL) was added thionyl chloride (0.5 mL, 0.004 mmol, 0.01 equiv) at ° C. under nitrogen atmosphere. The resulting mixture was stirred for overnight at ° C. under nitrogen atmosphere. The reaction was quenched with water (20 mL) at room temperature. The residue was purified by reverse flash chromatography (C18 silica gel column, eluting with 0% to 100% MeCN/water(0.1% FA) in 10 min with UV detector (254 nm)) to afford 4-(chloromethyl)-1-[1-methyl-4-(trifluoromethyl)imidazo]-2-yl]piperidine (85 mg, 44.13%) as a yellow solid. LC-MS (ESI) m/z 282 [M+H].

### Step 5: 2-(4-Cyclopropyl-6-Methoxypyrimidin-5-Yl)-8-({1-[1-Methyl-4-(Trifluoromethyl)Imidazol-2-Yl]Piperidin-4-Yl}Methyl)Pyrido[2,3-d]Pyrimidin-7-One

A solution of 4-(chloromethyl)-1-[1-methyl-4-(trifluoromethyl)imidazol-2-yl]piperidine (70 mg, 0.248 mmol, 1 equiv), 1,1,3,3-tetramethylguanidine (42.93 mg, 0.372 mmol, 1.5 equiv) and 2-(4-cyclopropyl-6-methoxypyrimidin-5-yl)-8H-pyrido[2,3-d]pyrimidin-7-one (73.38 mg, 0.248 mmol, 1 equiv) in DMF (27.24 mg, 0.372 mmol, 1.5 equiv) was stirred for overnight at 100° C. under nitrogen atmosphere. The resulting mixture was concentrated under reduced pressure. The residue was purified by reverse flash chromatography (C18 silica gel column, eluting with 0% to 100% MeCN/water(0.1% FA) in 10 min with UV detector (254 nm)) to afford 2-(4-cyclopropyl-6-methoxypyrimidin-5-yl)-8-({1-[1-methyl-4-(trifluoromethyl)imidazol-2-yl]piperidin-4-yl}methyl)pyrido[2,3-d]pyrimidin-7-one (12.3 mg, 9.08%) as an off-white solid. ¹H-NMR ¹H NMR (400 MHz, DMSO-d6) δ 9.24 (s, 1H), 8.71 (s, 1H), 8.10 (d, J=9.5 Hz, 1H), 7.51 (s, 1H), 6.85 (d, J=9.5 Hz, 1H), 4.29 (d, J=7.2 Hz, 2H), 3.87 (s, 3H), 3.46 (s, 3H), 3.22 (d, J=12.2 Hz, 3H), 2.62 (t, J=11.8 Hz, 2H), 2.06 (d, J=16.2 Hz, 1H), 1.80 (td, J=8.3, 4.4 Hz, 1H), 1.59 (d, J=12.7 Hz, 2H), 1.53-1.36 (m, 2H), 1.13-1.07 (m, 2H), 0.91 (dq, J=7.0, 3.4 Hz, 2H). LC-MS (ESI) m/z 541.10 [M+H].

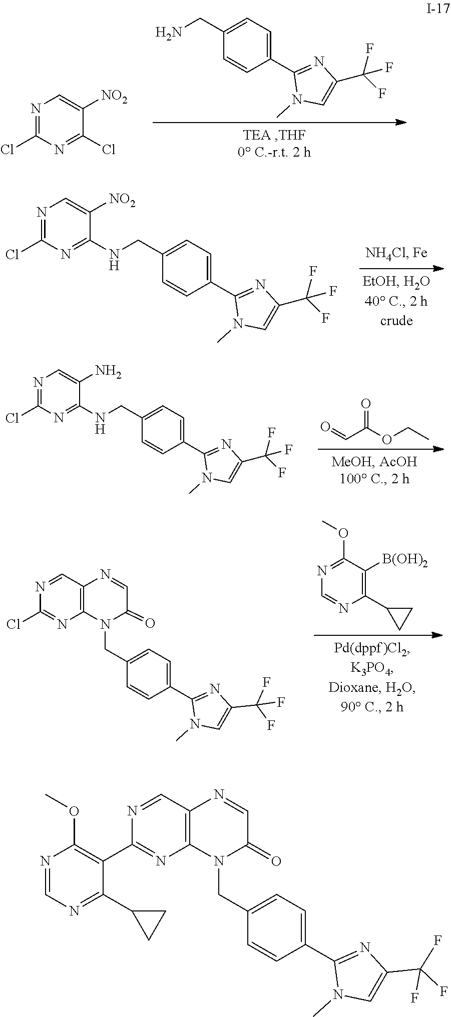

### Step 1: 2-Chloro-N-({4-[1-Methyl-4-(Trifluoromethyl)Imidazol-2-Yl]Phenyl}Methyl)-5-Nitropyrimidin-4-Amine

To a stirred solution of 2,4-dichloro-5-nitropyrimidine (400 mg, 2.062 mmol, 1 equiv) and TEA (626.03 mg, 6.186 mmol, 3 equiv) in THF (8 mL) was added (4-(1-methyl-4-(trifluoromethyl)-1H-imidazol-2-yl)phenyl)methanamine (547.17 mg, 2.268 mmol, 1.1 equiv) in THF (1 mL) dropwise at 0° C. The resulting mixture was stirred for 2 h at room temperature. The reaction was quenched by the addition of Water (20 mL) at room temperature. The resulting mixture was extracted with EtOAc (3×50 mL). The combined organic layers were washed with water (3×50 mL), dried over anhydrous Na₂SO₄, filtered and concentrated under reduced pressure. The residue was purified by reverse flash chromatography (C18 silica gel column, eluting with 0% to 100% MeCN/Water (0.1% FA) in 10 min with UV detector (254 nm)) to afford 2-chloro-N-({4-[1-methyl-4-(trifluoromethyl)imidazol-2-yl]phenyl}methyl)-5-nitropyrimidin-4-amine (160 mg, 18.80%) as a light yellow oil. LC-MS (ESI) m/z 413.7 [M+H].

### Step 2: 2-Chloro-N4-({4-[1-Methyl-4-(Trifluoromethyl)Imidazol-2-Yl]Phenyl}Methyl)Pyrimidine-4,5-Diamine

To a stirred solution of 2-chloro-N-({4-[1-methyl-4-(trifluoromethyl)imidazol-2-yl]phenyl}methyl)-5-nitropyrimidin-4-amine (50 mg, 0.121 mmol, 1 equiv) and NH 4 C1 (32.40 mg, mmol, 5 equiv) in ethanol (0.8 mL) and water (0.2 mL) was added Fe (54.12 mg, 0.968 mmol, 8 equiv) at room temperature. The resulting mixture was stirred for 2 h at 40° C. The resulting mixture was filtered, and the filter cake was washed with ethanol (3×10 mL). The filtrate was concentrated under reduced pressure. The crude product was used in the next step directly without further purification. LC-MS (ESI) m/z 383.7 [M+H].

### Step 3: 7-Chloro-1-({4-[1-Methyl-4-(Trifluoromethyl)Imidazol-2-Yl]Phenyl}Methyl)Pyrido[3,4-b]Pyrazin-2-One

To a stirred solution of 2-chloro-N4-({4-[1-methyl-4-(trifluoromethyl)imidazo]-2-yl]phenyl}methyl)pyrimidine-4,5-diamine (150 mg, 0.392 mmol, 1 equiv) and ethyl glyoxylate (120.02 mg, 1.176 mmol, 3 equiv) in MeOH (2 mL) was added AcOH (20.45 mg, 0.341 mmol, 1 equiv) dropwise at room temperature. The resulting mixture was stirred for 2 h at 100° C. and concentrated under reduced pressure. The residue was purified by reverse flash chromatography (C18 silica gel column, eluting with 0% to 100% MeCN/Water (0.1% FA) in 10 min with UV detector (254 nm)) to afford 7-chloro-1-({4-[1-methyl-4-(trifluoromethyl)imidazol-2-yl]phenyl}methyl)pyrido[3,4-b]pyrazin-2-one (40 mg, 24.3%) as a yellow oil. LC-MS (ESI) m/z 420.7 [M+H].

### Step 4: 2-(4-Cyclopropyl-6-Methoxypyrimidin-5-Yl)-8-({4-[1-Methyl-4-(Trifluoromethyl) Imidazol-2-Yl]Phenyl}Methyl) Pteridin-7-One

To a stirred solution of 2-chloro-8-({4-[1-methyl-4-(trifluoromethyl) imidazol-2-yl]phenyl}methyl)pteridin-7-one (50 mg, 0.119 mmol, 1 equiv) and 4-cyclopropyl-6-hydroxypyrimidin-5-ylboronic acid (32.08 mg, 0.178 mmol, 1.5 equiv) in 1,4-dioxane(0.4 mL) and H₂O (0.1 mL) were added K₃PO₄ (63.06 mg, 0.297 mmol, 2.5 equiv) and Pd(dppf)C12 (17.39 mg, 0.024 mmol, 0.2 equiv) at room temperature. The resulting mixture was stirred for 2 h at 90° C. under nitrogen atmosphere. The resulting mixture was filtered; the filter cake was washed with 1,4-dioxane (3×5 mL). The filtrate was concentrated under reduced pressure. The residue was purified by reverse flash chromatography (C18 silica gel column, eluting with 0% to 100% MeCN/Water (0.1% FA) in 10 min with UV detector (254 nm)) to afford 2-(4-cyclopropyl-6-methoxypyrimidin-5-yl)-8-({4-[1-methyl-4-(trifluoromethyl) imidazol-2-yl]phenyl}methyl) pteridin-7-one (16.1 mg, 23.35%) as a light yellow solid. ¹H NMR (400 MHz, DMSO-d6) δ 9.37 (d, J=1.5 Hz, 1H), 8.70 (s, 1H), 8.48 (s, 1H), 7.92 (s, 1H), 7.69-7.57 (m, 2H), 7.48 (d, J=8.0 Hz, 2H), 5.49 (s, 2H), 3.79 (dd, J=40.2, 1.4 Hz, 6H), 1.76 (m, 1H), 1.03 (m, 2H), 0.78 (m, 2H). LC-MS (ESI) m/z 535.1 [M+H].

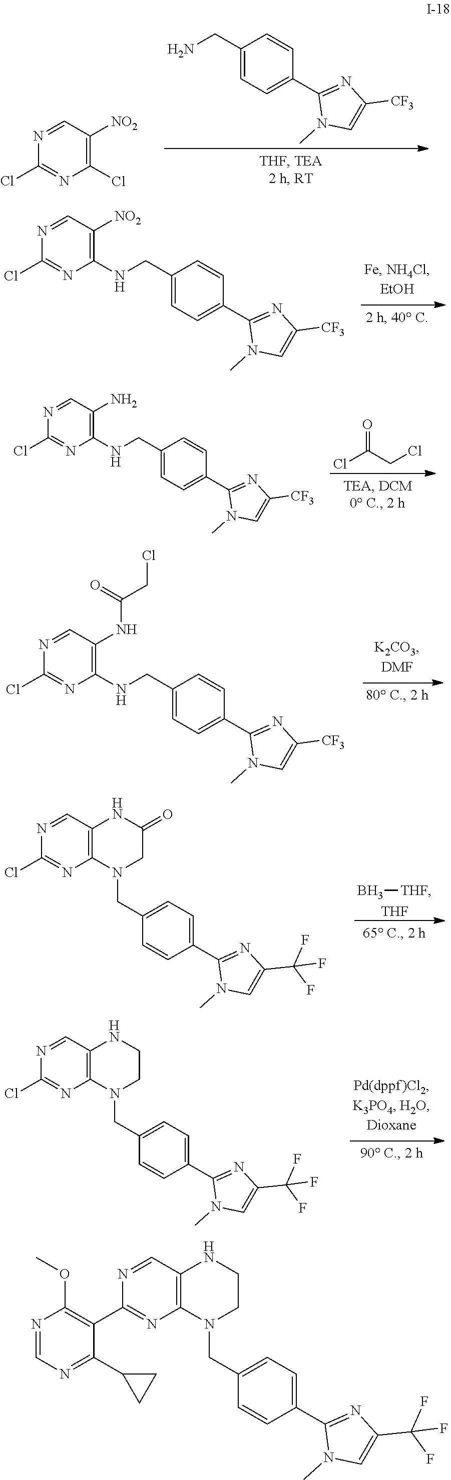

### Step 1 2-Chloro-N-({4-[1-Methyl-4-(Trifluoromethyl)Imidazol-2-Yl]Phenyl}Methyl)-5-Nitropyrimidin-4-Amine

A solution of 2,4-dichloro-5-nitropyrimidine (200 mg, 1.031 mmol, 1 equiv), TEA (310 mg, 3.063 mmol, 2.97 equiv) and 1-{4-[1-methyl-4-(trifluoromethyl)imidazo]-2-yl]phenyl}methanamine (310 mg, 1.215 mmol, 1.18 equiv) in THF(10 mL) was stirred for 2 h at room temperature under nitrogen atmosphere. The residue was purified by reverse flash chromatography (C18 silica gel column, eluting with 0% to 100% MeCN/water (0.1% FA) in 10 min with UV detector (254 nm)) to afford 2-chloro-N-({4-[1-methyl-4-(trifluoromethyl)imidazol-2-yl]phenyl}methyl)-5-nitropyrimidin-4-amine (336 mg, 78.95%) as a colorless oil. LC-MS (ESI) m/z 413 [M+H].

### Step 2: 2-Chloro-N4-({4-[1-Methyl-4-(Trifluoromethyl)Imidazol-2-Yl]Phenyl}Methyl)Pyrimidine-4,5-Diamine

A solution of 2-chloro-N-({4-[1-methyl-4-(trifluoromethyl)imidazo]-2-yl]phenyl}methyl)-5-nitropyrimidin-4-amine (375 mg, 0.909 mmol, 1 equiv), iron (400 mg, 7.163 mmol, 7.88 equiv), NH 4 C1 (240 mg, 4.487 mmol, 4.94 equiv) and H₂O (1 mL, 55.509 mmol, 61.10 equiv) in EtOH (4 mL) was stirred for 2 h at 40° C. under nitrogen atmosphere. The resulting mixture was filtered, the filter cake was washed with MeOH (2×10 mL). The filtrate was concentrated under reduced pressure. The residue was purified by silica gel column chromatography, (eluting with PE/EA (1:1)) to afford 2-chloro-N4-({4-[1-methyl-4-(trifluoromethyl)imidazo]-2-yl]phenyl methyl)pyrimidine-4,5-diamine (175 mg, 50.32%) as a yellow solid. LC-MS (ESI) m/z 383 [M+H].

### Step 3: 2-Chloro-N-{2-Chloro-4-1({4-[1-Methyl-4-(Trifluoromethyl)Imidazol-2-Yl]Phenyl}Methyl)Amino]Pyrimidin-5-Yl}Acetamide

A solution of 2-chloro-N4-({4-[1-methyl-4-(trifluoromethyl)imidazol-2-yl]phenyl methyl)pyrimidine-4,5-di amine (200 mg, 0.522 mmol, 1 equiv) in DCM (2 mL, 31.461 mmol, 60.21 equiv) was treated with TEA (1.4 mL, 10.072 mmol, 19.28 equiv) for 5 min at under nitrogen atmosphere followed by the addition of chloroacetyl chloride (150 mg, 1.328 mmol, 2.54 equiv) dropwise at 0° C. The resulting mixture was stirred for 2 h at room temperature under nitrogen atmosphere. The resulting mixture was concentrated under reduced pressure. The residue was purified by reverse flash chromatography (C18 silica gel column, eluting with 0% to 100% MeCN/water(0.1% FA) in 10 min with UV detector (254 nm)) to afford 2-chloro-N-{2-chloro-4-[({4-[1-methyl-4-(trifluoromethyl)imidazol-2-yl]phenyl methyl)amino]pyrimidin-5-yl acetamide (100 mg, 41.67%) as a colorless oil. LC-MS (ESI) m/z 459 [M+H].

### Step 4: 2-Chloro-8-({4-[1-Methyl-4-(Trifluoromethyl)Imidazol-2-Yl]Phenyl}Methyl)-5,7-Dihydropteridin-6-One

A solution of 2-chloro-N-{2-chloro-4-[({4-[1-methyl-4-(trifluoromethyl)imidazol-2-yl]phenyl methyl)amino]pyrimidin-5-yl acetamide (130 mg, 0.283 mmol, 1 equiv) and K₂CO₃ (78.24 mg, 0.566 mmol, 2 equiv) in DMF (10 mL, 129.215 mmol, 456.48 equiv) was stirred for 2h at 80° C. under nitrogen atmosphere. The mixture was allowed to cool down to room temperature. The residue was purified by reverse flash chromatography (C18 silica gel column, eluting with 0% to 100% MeCN/water(0.1% FA) in 10 min with UV detector (254 nm)) to afford 2-chloro-8-({4-[1-methyl-4-(trifluoromethyl)imidazol-2-yl]phenyl}methyl)-5,7-dihydropteridin-6-one (23 mg, 19.22%) as an off-white solid. LC-MS (ESI) m/z 423 [M+H].

### Step 5: 2-Chloro-8-({4-[1-Methyl-4-(Trifluoromethyl)Imidazol-2-Yl]Phenyl}Methyl)-6,7-Dihydro-511-Pteridine

A solution of 2-chloro-8-({4-[1-methyl-4-(trifluoromethyl)imidazo]-2-yl]phenyl}methyl)-5,7-dihydropteridin-6-one (20 mg, 0.047 mmol, 1 equiv) and BH₃-THF (1.4 mL, 14.629 mmol, 309.25 equiv) in THF (2 mL, 24.686 mmol, 521.85 equiv) was stirred for 2h at 65° C. under nitrogen atmosphere. The reaction was quenched by the addition of MeOH (4 mL) at 0° C. The resulting mixture was concentrated under reduced pressure. The residue was purified by reverse flash chromatography (C18 silica gel column, eluting with 0% to 100% MeCN/water(0.1% FA) in 10 min with UV detector (254 nm)) to afford 2-chloro-8-({4-[1-methyl-4-(trifluoromethyl)imidazo]-2-yl]phenyl methyl)-6,7-dihydro-5H-pteridine (10 mg, 51.71%) as a colorless solid. LC-MS (ESI) m/z 409 [M+H].

### Step 6: 2-(4-Cyclopropyl-6-Methoxypyrimidin-5-Yl)-8-({4-[1-Methyl-4-(Trifluoromethyl)Imidazol-2-Yl]Phenyl}Methyl)-6,7-Dihydro-511-Pteridine

A solution of 2-chloro-8-({4-[1-methyl-4-(trifluoromethyl)imidazo]-2-yl]phenyl}methyl)-6,7-dihydro-5H-pteridine (50 mg, 0.122 mmol, 1 equiv), 4-cyclopropyl-6-methoxypyrimidin-5-ylboronic acid (28.47 mg, 0.146 mmol, 1.2 equiv), Pd(dppf)Cl₂ (17.90 mg, mmol, 0.2 equiv), H₂O (1 mL, 55.509 mmol, 453.86 equiv) and K₃PO₄ (38.94 mg, 0.183 mmol, 1.5 equiv) in dioxane (4 mL) was stirred for 2 h at 90° C. under nitrogen atmosphere. The mixture was allowed to cool down to room temperature. The residue was purified by reverse flash chromatography (C18 silica gel column, eluting with 0% to 100% MeCN/water(0.1% FA) in 10 min with UV detector (254 nm)) to afford 2-(4-cyclopropyl-6-methoxypyrimidin-5-yl)-8-({4-[1-methyl-4-(trifluoromethyl)imidazol-2-yl]phenyl}methyl)-6,7-dihydro-5H-pteridine (1.7 mg, 2.47%) as an off-white solid. LC-MS (ESI) m/z 523 [M+H]

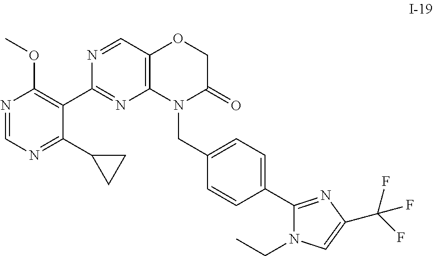 [SMILES: CCn1cc(C(F)(F)F)nc1-c1ccc(CN2C(=O)COc3cnc(-c4c(OC)ncnc4C4CC4)nc32)cc1]

1-19 was made in an analogous way as 1-5. ¹H-NMR (400 MHz, DMSO-d6) δ 8.64 (s, 1H), 8.53 (s, 1H), 8.02 (d, J=1.4 Hz, 1H), 7.61-7.53 (m, 2H), 7.47 (d, J=8.1 Hz, 2H), 5.24 (s, 2H), 5.05 (s, 2H), 4.05 (q, J=7.3 Hz, 2H), 3.81 (s, 3H), 1.68 (tt, J=8.2, 4.6 Hz, 1H), 1.30 (t, J=7.3 Hz, 3H), 0.98 (q, J=3.4 Hz, 2H), 0.80-0.72 (m, 2H). LC-MS (ESI) m/z 552 [M+H]. 1-20

1-20 was synthesized in an analogous was as I-1

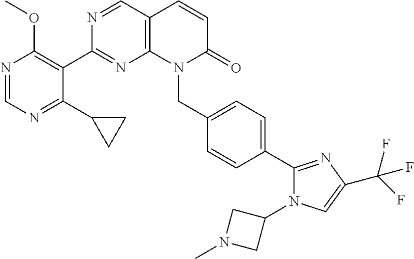 [SMILES: COc1ncnc(C2CC2)c1-c1ncc2ccc(=O)n(Cc3ccc(-c4nc(C(F)(F)F)cn4C4CN(C)C4)cc3)c2n1]

¹H NMR: (300 MHz, DMSO-d6) δ 9.31 (s, 1H), 8.70 (s, 1H), 8.48 (s, 1H), 8.22-8.12 (m, 1H), 7.45 (q, J=8.3 Hz, 4H), 6.91 (d, J=9.5 Hz, 1H), 5.58 (s, 2H), 5.05 (t, J=7.3 Hz, 1H), 4.03 (d, J=8.5 Hz, 2H), 3.94 (s, 1H), 3.84 (s, 3H), 2.63 (s, 3H), 1.73 (td, J=7.9, 4.1 Hz, 1H), 1.02 (q, J=3.3 Hz, 2H), 0.83-0.72 (m, 2H). LC-MS (ESI) m/z 589.05 [M+H].

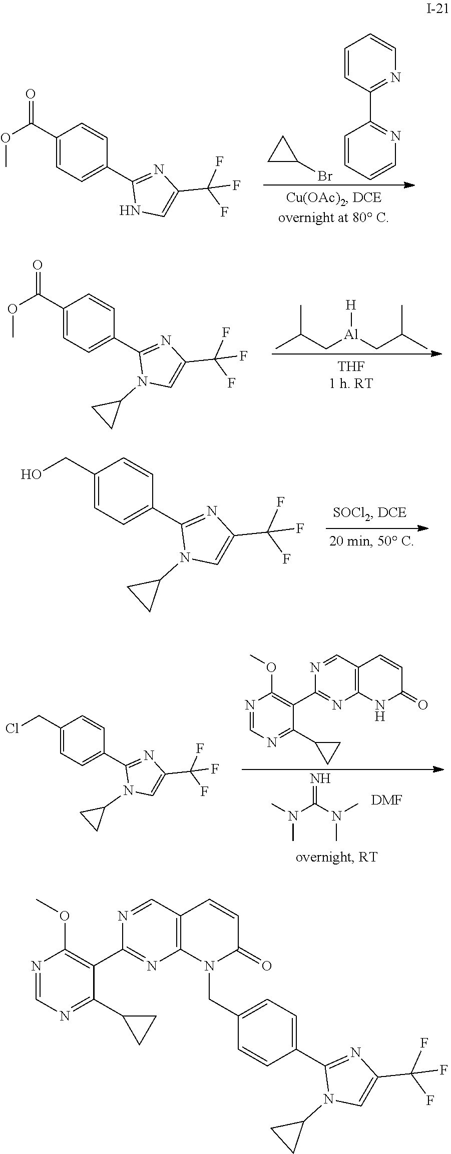

### Step 1: 4-[1-Cyclopropyl-4-(Trifluoromethyl)Imidazol-2-Yl]Benzoate

A mixture of methyl 4-[4-(trifluoromethyl)-1H-imidazol-2-yl]benzoate (5 g, 18.504 mmol, 1 equiv), bipyridyl (8670.34 mg, 55.512 mmol, 3 equiv), bromocyclopropane (6715.70 mg, 55.512 mmol, 3 equiv) and Cu(OAc) 2 (10. 08 g, 55.512 mmol, 3 equiv) in DCE (50 mL) was stirred for overnight at 80° C. under oxygen atmosphere. The resulting mixture was diluted with DCM (100 mL). The resulting mixture was washed with 3×100 mL of water. The resulting organic phase was concentrated under reduced pressure. The residue was purified by reverse flash chromatography (C18 silica gel column, eluting with 0% to 100% MeCN/water (10 mmol/L NH₄HCO₃) in 10 min with UV detector (254 nm)) to afford methyl 4-[1-cyclopropyl-4-(trifluoromethyl)imidazol-2-yl]benzoate (430 mg, 7.49%) as a brown solid. LC-MS (ESI) m/z 311.10 [M+H].

### Step 2: {4-[1-Cyclopropyl-4-(Trifluoromethyl)Imidazol-2-Yl]Phenyl}Methanol

To a stirred solution of methyl 4-[1-cyclopropyl-4-(trifluoromethyl)imidazo]-2-yl]benzoate (160 mg, 0.516 mmol, 1 equiv) in THF (5 mL) was added diisobutylaluminium hydride (2.58 mL, 2.580 mmol, 5 equiv., 1 M in hexanes) dropwise at room temperature under nitrogen atmosphere. The resulting mixture was stirred for 1 h at room temperature under nitrogen atmosphere. The reaction was quenched with sat. NH₄Cl (aq.) at room temperature. The resulting mixture was filtered, the filter cake was washed with water (3×10 mL). The aqueous layer was extracted with EtOAc (3×10 mL). The combined organic layers were dried over anhydrous Na₂SO₄, filtered and concentrated under reduced pressure. The residue was purified by reverse flash chromatography (C18 silica gel column, eluting with 10% to 50% MeCN/water (10 mmol/L NH₄HCO₃) in 10 min with UV detector (254 nm)) to afford {4-[1-cyclopropyl-4-(trifluoromethyl)imidazol-2-yl]phenyl}methanol (120 mg, 82.44%) as a brown oil. LC-MS (ESI) m/z 283.10 [M+H].

### Step 3: 2-[4-(Chloromethyl)Phenyl]-1-Cyclopropyl-4-(Trifluoromethyl)Imidazole

To a stirred solution of {4-[1-cyclopropyl-4-(trifluoromethyl)imidazo]-2-yl]phenyl}methanol (120 mg, 0.425 mmol, 1 equiv) in DCE (5 mL) was added thionyl chloride (252.87 mg, 2.125 mmol, 5 equiv) dropwise at room temperature. The resulting mixture was stirred for 20 min at 50° C. under nitrogen atmosphere. The resulting mixture was diluted with DCM (10 mL). The resulting mixture washed with water (3×10 mL), dried over anhydrous Na₂SO₄, filtered and concentrated under reduced pressure to afford 2-[4-(chloromethyl)phenyl]-1-cyclopropyl-4-(trifluoromethyl)imidazole (110 mg, 86.04%) as a brown oil. LC-MS (ESI) m/z 301.06 [M+H].

### Step 4: Synthesis of 8-({4-[1-Cyclopropyl-4-(Trifluoromethyl)Imidazol-2-Yl]Phenyl}Methyl)-2-(4-Cyclopropyl-6-Methoxypyrimidin-5-Yl)Pyrido[2,3-d]Pyrimidin-7-One

A mixture of 2-[4-(chloromethyl)phenyl]-1-cyclopropyl-4-(trifluoromethyl)imidazole (28 mg, 0.093 mmol, 1 equiv), 2-(4-cyclopropyl-6-methoxypyrimidin-(27.50 mg, 0.093 mmol, 1 equiv) and 1,1,3,3-tetramethylguanidine (16.09 mg, 0.140 mmol, 1.5 equiv) in DMF (2 mL) was stirred for overnight at room temperature under nitrogen atmosphere. The crude product was purified by reverse flash chromatography (C18 silica gel column, eluting with 10% to 50% MeCN/water (10 mmol/L NH₄HCO₃) in 10 min with UV detector (254 nm)) afford 8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (10 mg, 18.48%) as an off-white solid. ¹H NMR: (400 MHz, DMSO-d6) δ 9.29 (d, J=2.6 Hz, 1H), 8.69 (s, 1H), 8.16 (dd, J=9.5, 2.6 Hz, 1H), 7.91 (d, J=1.4 Hz, 1H), 7.84-7.78 (m, 2H), 7.39 (d, J=8.0 Hz, 2H), 6.91 (dd, J=9.5, 3.5 Hz, 1H), 5.57 (s, 2H), 3.85-3.79 (m, 3H), 3.70 (d, J=4.5 Hz, 1H), 1.73 (dd, J=8.2, 4.0 Hz, 1H), 1.07-0.83 (m, 6H), (dt, J=7.1, 3.5 Hz, 2H). LC-MS (ESI) m/z 560.10 [M+H].

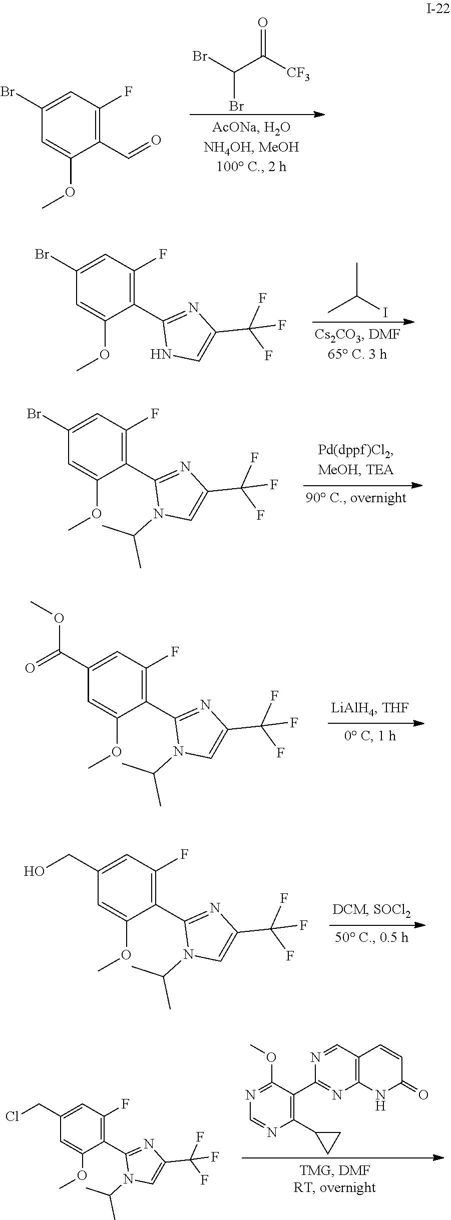

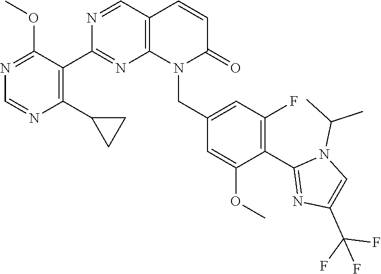 [SMILES: COc1cc(Cn2c(=O)ccc3cnc(-c4c(OC)ncnc4C4CC4)nc32)cc(F)c1-c1nc(C(F)(F)F)cn1C(C)C]

### Step 1: 2-(4-Bromo-2-Fluoro-6-Methoxyphenyl)-4-(Trifluoromethyl)-1H-Imidazole

A mixture of 3,3-dibromo-1,1,1-trifluoroprop an-2-one (259.38 mg, 0.961 mmol, 1.12 equiv) and AcONa (78.85 mg, 0.961 mmol, 1.12 equiv) in H₂O (0.4 mL, 2 equiv) was stirred for 1 h at 100° C. under nitrogen atmosphere. The mixture was cooled down to room temperature. To the above mixture were added 4-bromo-2-fluoro-6-methoxybenzaldehyde (200 mg, 0.858 mmol, 1 equiv), MeOH (3 mL, 15.2 equiv) and NH 4 OH (0.7 mL, 17.976 mmol, 20.95 equiv) at room temperature. The resulting mixture was stirred for additional 40 min at room temperature. Then the resulting mixture was stirred for 2 h at 100° C. under nitrogen atmosphere. The reaction was quenched with water at 0° C. The resulting mixture was extracted with EtOAc (3×10 mL). The combined organic layers were washed with brine (2×5 mL), dried over anhydrous Na₂SO₄, filtered and concentrated under reduced pressure. The residue was dissolved in MeOH (3 mL). The residue was purified by reverse flash chromatography (C18 silica gel column, eluting with 0% to 100% MeCN/Water (0.1% FA) in 10 min with UV detector (254 nm)) to afford 2-(4-bromo-2-fluoro-6-methoxyphenyl)-4-(trifluoromethyl)-1H-imidazole (90 mg, as a light yellow solid. LC-MS (ESI) m/z 338.9 [M+H]

### Step 2: 2-(4-Bromo-2-Fluoro-6-Methoxyphenyl)-1-Isopropyl-4-(Trifluoromethyl)Imidazole

To a stirred 2-(4-bromo-2-fluoro-6-methoxyphenyl)-4-(trifluoromethyl)-1H-imidazole (90 mg, 0.265 mmol, 1 equiv) in dimethylformamide (1 mL, 20 equiv) were added caesio methaneperoxoate caesium (173.49 mg, 0.530 mmol, 2 equiv) and 2-iodopropane (135.36 mg, 0.795 mmol, 3 equiv) at room temperature under nitrogen atmosphere. The resulting mixture was stirred for 3 h at 65° C. under nitrogen atmosphere. The resulting mixture was filtered, the filter cake was washed with THF (3×5 mL). The filtrate was concentrated under reduced pressure. The residue was dissolved in THF (2 mL). The residue was purified by reverse flash chromatography (C18 silica gel column, eluting with 0% to 100% MeCN/Water (0.1% FA) in 10 min with UV detector (254 nm)) to afford 2-(4-bromo-2-fluoro-6-methoxyphenyl)-1-isopropyl-4-(trifluoromethyl)imidazole (70 mg, 69.19%) as a yellow solid. LC-MS (ESI) m/z 383.1 [M+H]

### Step 3: Methyl 3-Fluoro-4-[1-Isopropyl-4-(Trifluoromethyl)Imidazol-2-Yl]-5-Methoxybenzoate

To a stirred 2-(4-bromo-2-fluoro-6-methoxyphenyl)-1-isopropyl-4-(trifluoromethyl)imidazole (100 mg, 0.262 mmol, 1 equiv) in MeOH (8 mL, 197.591 mmol) were added Pd(dppf)Cl₂ (35 mg, 0.048 mmol, 0.18 equiv) and TEA (1 mL, 7.194 mmol, 27.42 equiv) at room temperature under carbon monoxide atmosphere. The resulting mixture was stirred for overnight at 90° C. under carbon monoxide atmosphere. The resulting mixture was concentrated under reduced pressure. The residue was dissolved in THF (2 mL). The residue was purified by reverse flash chromatography (C18 silica gel column, eluting with 0% to 100% MeCN/Water (0.1% FA) in 10 min with UV detector (254 nm)) to afford methyl 3-fluoro-4-[1-isopropyl-4-(trifluoromethyl)imidazol-2-yl]-5-methoxybenzoate (70 mg, 74.05%) as a yellow solid. LC-MS (ESI) m/z 361.1 [M+H]

### Step 4: {3-Fluoro-4-[1-Isopropyl-4-(Trifluoromethyl)Imidazol-2-Yl]-5-Methoxyphenyl}Methanol

To a stirred mixture of methyl 3-fluoro-4-(1-isopropyl-4-(trifluoromethyl)-1H-imidazol-2-yl)-5-methoxybenzoate (70 mg, 0.202 mmol, 1 equiv) in THF (3 mL, 37.028 mmol, 190.59 equiv) was added LiAlH₄ (22.12 mg, 0.582 mmol, 3.0 equiv) in portions at under nitrogen atmosphere. The resulting mixture was stirred for 1 h at under nitrogen atmosphere. The reaction was quenched with 10H2O.Na₂SO₄ at 0° C. The resulting mixture was filtered, the filter cake was washed with THF (2×2 mL). The filtrate was concentrated under reduced pressure. The residue was purified by reverse flash chromatography (C18 silica gel column, eluting with 0% to 100% MeCN/Water (0.1% FA) in 10 min with UV detector (254 nm)) to afford {3-fluoro-4-[1-isopropyl-4-(trifluoromethyl)imidazol-2-yl]-5-methoxyphenyl}methanol (50 mg, 77.45%) as a brown solid. LC-MS (ESI) m/z 333.1 [M+H]

### Step 5: 2-[4-(Chloromethyl)-2-Fluoro-6-Methoxyphenyl]-1-Methyl-4-(Trifluoromethyl)Imidazole

To a stirred {3-fluoro-4-[1-isopropyl-4-(trifluoromethyl)imidazol-2-yl]-5-methoxyphenyl}methanol (100 mg, 0.301 mmol, 1 equiv) in DCM (2 mL, 0.024 mmol) was added SOCl₂ (107.5 mg, 0.904 mmol, 3.00 equiv) at 50° C. under nitrogen atmosphere. The resulting mixture was stirred for 30 min at 50° C. under nitrogen atmosphere. The reaction was quenched with Water/Ice at 0° C. The resulting mixture was extracted with CH₂Cl₂ (3×5 mL). The combined organic layers were washed with brine (3×5 mL), dried over anhydrous Na₂SO₄, filtered and concentrated under reduced pressure to afford 2-[4-(chloromethyl)-2-fluoro-6-methoxyphenyl]-1-methyl-4-(trifluoromethyl)imidazole (110 mg, 113.28%) as a brown solid. The crude product was used in the next step directly without further purification. LC-MS (ESI) m/z 351.1 [M+H]

### Step 6: 2-(4-Cyclopropyl-6-Methoxypyrimidin-5-Yl)-8-({3-Fluoro-4-[1-Isopropyl-4-(Trifluoromethyl)Imidazol-2-Yl]-5-Methoxyphenyl}Methyl)Pyrido[2,3-d]Pyrimidin-7-One

A solution of 2-[4-(chloromethyl)-2-fluoro-6-methoxyphenyl]-1-isopropyl-4-(trifluoromethyl)imidazole (21.3 mg, 0.061 mmol, 1 equiv), 2-(4-cyclopropyl-6-methoxypyrimidin-5-yl)-8H-pyrido[2,3-d]pyrimidin-7-one (17.93 mg, 0.061 mmol, 1 equiv) and 1,1,3,3-tetramethylguanidine (10.49 mg, 0.091 mmol, 1.5 equiv) in DMF (2 mL, 25,845 mmol, 425.55 equiv) was stirred for overnight at room temperature under nitrogen atmosphere. The crude product was purified by Prep-HPLC with the following conditions (Column: Xselect CSH F-Phenyl OBD column, 19*250 mm, 51 μm; Mobile Phase A: Water(0.1% FA), Mobile Phase B: MeOH-HPLC; Flow rate: 25 mL/min; Gradient: 60% B to 80% B in 8 min, 80% B; Wave Length: 220 nm; RT 1(min): 7.56;) to afford 2-(4-cyclopropyl-6-methoxypyrimidin-5-yl)-8-({3-fluoro-4-[1-isopropyl-4-(trifluoromethyl)imidazol-2-yl]-5-methoxyphenyl}methyl)pyrido[2,3-d]pyrimidin-7-one (9 mg, 23.41%) as an off-white solid. ¹H NMR (400 MHz, DMSO-d6) δ 9.30 (s, 1H), 8.69 (s, 1H), 8.18 (t, J=4.8 Hz, 2H), 7.04 (s, 1H), 6.92 (d, J=9.5 Hz, 1H), 6.72 (d, J=9.6 Hz, 1H), 5.56 (s, 2H), 3.91 (p, J=6.9 Hz, 1H), 3.82 (s, 3H), 3.69 (s, 3H), 1.75 (dq, J=8.7, 5.2, 4.4 Hz, 1H), 1.35 (d, J=6.7 Hz, 3H), 1.23 (d, J=6.7 Hz, 3H), 1.02 (d, J=4.6 Hz, 2H), 0.77 (d, J=7.5 Hz, 2H). LC-MS (ESI) m/z 610.05 [M+H].

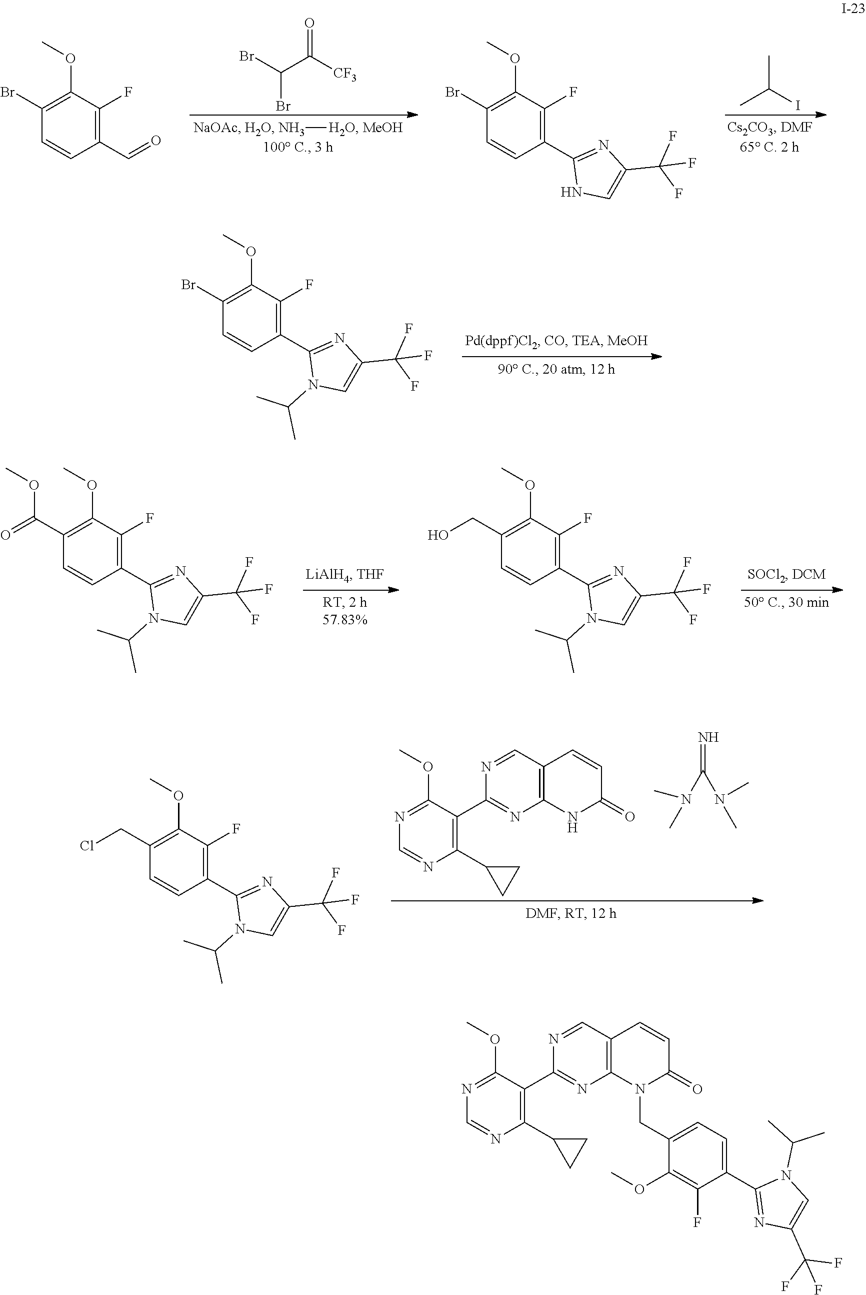

### Step 1: 2-(4-Bromo-2-Fluoro-3-Methoxyphenyl)-4-(Trifluoromethyl)-1H-Imidazole

A mixture of 3,3-dibromo-1,1,1-trifluoropropan-2-one (1.30 g, 4.806 mmol, 1.12 equiv) and NaOAc (0.97 g, 11.879 mmol, 1.12 equiv) in H₂O (2.00 mL, 111.008 mmol, 25.87 equiv) was stirred for 1 h at 100° C. under nitrogen atmosphere. To the above mixture was added 4-bromo-2-fluoro-3-methoxybenzaldehyde (1 g, 4.291 mmol, 1 equiv) and MeOH (30 mL, 15.2 equiv) and NH₃—H₂O (3.50 mL, 89.896 mmol, 20.95 equiv) at room temperature. The resulting mixture was stirred for additional 40 min at room temperature. The resulting mixture was stirred for 2 h at 100° C. under nitrogen atmosphere. The reaction was quenched with Water at room temperature. The resulting mixture was extracted with EtOAc (3×10 mL). The combined organic layers were washed with water (3×5 mL), dried over anhydrous Na₂SO₄.After filtration, the filtrate was concentrated under reduced pressure. The residue was purified by reverse flash chromatography (C18 silica gel column, eluting with 0% to 100% MeCN/water (10 mmol/L NH₄HCO₃) in 10 min with UV detector (254 nm)) to afford 2-(4-bromo-2-fluoro-3-methoxyphenyl)-4-(trifluoromethyl)-1H-imidazole (880 mg, 60.48%) as a yellow solid. LC-MS (ESI) m/z 339 [M+H]

### Step 2: 2-(4-Bromo-2-Fluoro-3-Methoxycyclohexyl)-1-Isopropyl-4-(Trifluoromethyl)Imidazolidine

To a stirred mixture of 2-(4-bromo-2-fluoro-3-methoxycyclohexyl)-4-(trifluoromethyl)imidazolidine (880 mg, 2.520 mmol, 1 equiv) and Cs₂CO₃ (1642.31 mg, 5.040 mmol, 2 equiv) in DMF(4 ml) was added 2-iodopropane (1285.29 mg, 7.560 mmol, 3 equiv) at room temperature under nitrogen atmosphere. The resulting mixture was stirred for 2 h at resulting mixture was filtered, the filter cake was washed with MeCN (1×1 mL). The filtrate was concentrated under reduced pressure. The residue was purified by reverse flash chromatography (C18 silica gel column, eluting with 10% to 50% MeCN/water (10 mmol/L NH₄HCO₃) in 10 min with UV detector (254 nm)) to afford 2-(4-bromo-2-fluoro-3-methoxycyclohexyl)-1-isopropyl-4-(trifluoromethyl)imidazolidine (880 mg, 89.24%) as a yellow solid. LC-MS (ESI) m/z 381 [M+H]

### Step 3: Methyl 3-Fluoro-4-[1-Isopropyl-4-(Trifluoromethyl)Imidazol-2-Yl]-2-Methoxybenzoate

To a stirred mixture of 2-(4-chloro-2-fluoro-3-methoxyphenyl)-1-isopropyl-4-(trifluoromethyl)imidazole (860 mg, 2.554 mmol, 1 equiv) and TEA (1550.76 mg, 15.324 mmol, 6 equiv) in MeOH (16.00 mL, 395.181 mmol, 154.72 equiv) was added Pd(dppf)Cl₂ (186.89 mg, mmol, 0.1 equiv) at room temperature under carbon monoxide atmosphere. The resulting mixture was stirred for additional 12 h at 90° C. The resulting mixture was filtered, the filter cake was washed with MeOH (3×4 mL). The filtrate was concentrated under reduced pressure. The residue was purified by reverse flash chromatography (C18 silica gel column, eluting with 10% to 50% MeCN/water (10 mmol/L NH₄HCO₃) in 10 min with UV detector (254 nm)) to afford methyl 3-fluoro-4-[1-isopropyl-4-(trifluoromethyl)imidazol-2-yl]-2-methoxybenzoate (600 mg, as a yellow oil. LC-MS (ESI) m/z 361 [M+H]

### Step 4: {3-Fluoro-4-[1-Isopropyl-4-(Trifluoromethyl)Imidazol-2-Yl]-2-Methoxyphenyl}Methanol

To a stirred mixture of methyl 3-fluoro-4-[1-isopropyl-4-(trifluoromethyl)imidazol-2-yl]-2-methoxybenzoate (600 mg, 1.665 mmol, 1 equiv) in THF (8.82 mL, 108.891 mmol, 65.40 equiv) was added LiAlH₄ (189.59 mg, 4.995 mmol, 3 equiv) in portions at room temperature under nitrogen atmosphere. The resulting mixture was stirred for 2 h at room temperature. The reaction was quenched with 10H2O.Na₂SO₄ at 0° C. The resulting mixture was filtered, the filter cake was washed with THF (2×2 mL). The filtrate was concentrated under reduced pressure. The residue was purified by silica gel column chromatography, (eluting with PE/EA (1:1)) to afford {3-fluoro-4-[1-isopropyl-4-(trifluoromethyl)imidazol-2-yl]-2-methoxyphenyl}methanol (320 mg, 57.83%) as a white oil. LC-MS (ESI) m/z 333 [M+H]Step 5: 2-[4-(chloromethyl)-2-fluoro-3-methoxyphenyl]-1-isopropyl-4-(trifluoromethyl)imidazole

To a stirred mixture of {3-fluoro-4-[1-isopropyl-4-(trifluoromethyl)imidazo]-2-yl]-2-methoxyphenyl}methanol (30 mg, 0.090 mmol, 1 equiv) in DCM (1 mL, 15.731 mmol, 174.24 equiv) was added SOCl₂ (0.02 mL, 0.270 mmol, 3 equiv) at room temperature under nitrogen atmosphere. The reaction was quenched with saturated NaHCO₃(aq.) and extracted with CH₂Cl₂ (3×mL). The combined organic layers were washed with water (2×2 mL), dried over anhydrous Na₂SO₄, filtered and concentrated under reduced pressure. The crude was used in the next step directly without further purification. LC-MS (ESI) m/z 351 [M+H]

### Step 6: 2-(4-Cyclopropyl-6-Methoxypyrimidin-5-Yl)-8-({3-Fluoro-4-[1-Isopropyl-4-(Trifluoromethyl)Imidazol-2-Yl]-2-Methoxyphenyl}Methyl)Pyrido[2,3-d]Pyrimidin-7-One

To a stirred mixture of 2-[4-(chloromethyl)-2-fluoro-3-methoxyphenyl]-1-isopropyl-4-(trifluoromethyl)imidazole (40 mg, 0.114 mmol, 0.9 equiv) and 2-(4-cyclopropyl-6-methoxypyrimidin-5-yl)-8H-pyrido[2,3-d]pyrimidin-7-one (37.42 mg, 0.127 mmol, 1 equiv) in DMF (4.44 mL, 57.407 mmol, 453.21 equiv) was added 1,1,3,3-tetramethylguanidine (21.89 mg, 0.190 mmol, 1.5 equiv) at room temperature under nitrogen atmosphere. The resulting mixture was stirred for 2 h at room temperature. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({3-fluoro-4-[1-isopropyl-4-(trifluoromethyl)imidazol-2-yl]-2-methoxyphenyl}methyl)pyrido[2,3-d]pyrimidin-7-one (20 mg, as a white solid. ¹H NMR (400 MHz, DMSO-d6) δ 9.31 (s, 1H), 8.66 (s, 1H), 8.25-8.16 (m, 2H), 7.08 (t, J=7.3 Hz, 1H), 6.93 (d, J=9.6 Hz, 1H), 6.70 (d, J=8.1 Hz, 1H), 5.59 (s, 2H), 4.17-4.09 (m, 1H), 3.89 (s, 3H), 3.78 (s, 3H), 1.74-1.67 (m, 1H), 1.35 (d, J=6.6 Hz, 6H), 1.01-0.95 (m, 2H), 0.70 (dd, J=7.9, 3.4 Hz, 2H). LC-MS (ESI) m/z 610.25 [M+H]

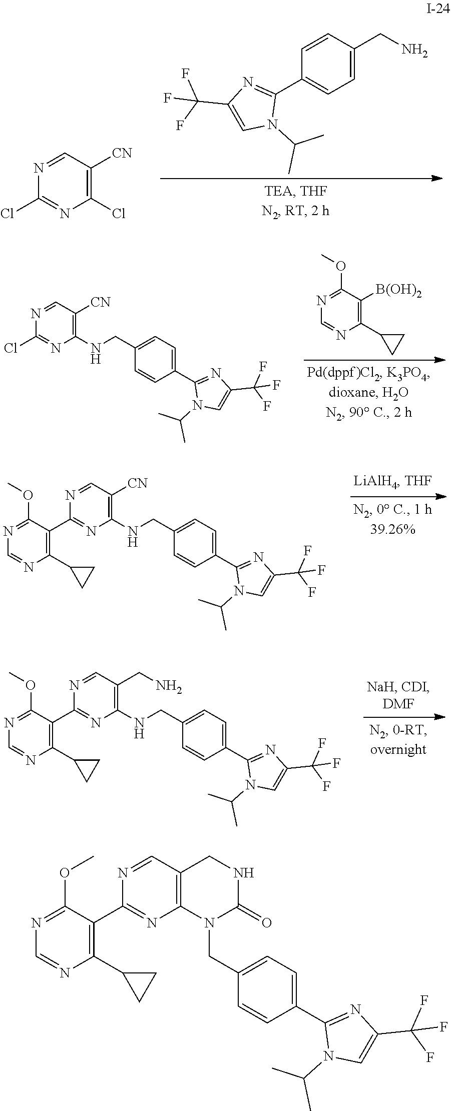

### Step 1: 2-Chloro-4-1({4-[1-Isopropyl-4-(Trifluoromethyl)Imidazol-2-Yl]Phenyl}Methyl)Amino]Pyrimidine-5-Carbonitrile

To a stirred solution of 2,4-dichloropyrimidine-5-carbonitrile (1.7 g, 9.771 mmol, 1 equiv) and 1-{4-[1-isopropyl-4-(trifluoromethyl)imidazo]-2-yl]phenyl}methanamine (2.77 g, 9.771 mmol, 1 equiv) in THF (20 mL) was added TEA (1.98 g, 19.542 mmol, 2 equiv) dropwise at room temperature. The resulting mixture was stirred for 2 h at room temperature under nitrogen atmosphere. The residue was purified by reverse flash chromatography (C18 silica gel column, eluting with 0% to 100% MeCN/water (0.1% FA) in 10 min with UV detector (254 nm)) to afford 2-chloro-44({4-[1-isopropyl-4-(trifluoromethyl)imidazol-2-yl]phenyl}methyl)amino]pyrimidine-5-carbonitrile (900 mg, 21.89%) as an off-white solid. LC-MS (ESI) m/z 421.8 [M+H]

### Step 2: 4′-Cyclopropyl-4-1({4-[1-Isopropyl-4-(Trifluoromethyl)Imidazol-2-Yl]Phenyl}Methyl)Amino]-6′-Methoxy-12,5′-Bipyrimidine]-5-Carbonitrile

To a stirred solution of 2-chloro-44({4-[1-isopropyl-4-(trifluoromethyl)imidazo]-2-yl]phenyl}methyl)amino]pyrimidine-5-carbonitrile (438 mg, 1.041 mmol, 1 equiv) and 4-cyclopropyl-6-methoxypyrimidin-5-ylboronic acid (302.88 mg, 1.561 mmol, 1.5 equiv) in H₂O (0.5 mL) and dioxane (5 mL) were added K₃PO₄ (552.33 mg, 2.603 mmol, 2.5 equiv) and Pd(dppf)Cl₂ (152.32 mg, 0.208 mmol, 0.2 equiv) at room temperature under nitrogen atmosphere. The resulting mixture was stirred for 2 h at 90° C. under nitrogen atmosphere. The resulting mixture was filtered, the filter cake was washed with THF (3×5 mL) and concentrated under reduced pressure. The residue was purified by reverse flash chromatography (C18 silica gel column, eluting with 0% to 100% MeCN/water (0.1% FA) in 10 min with UV detector (254 nm)) to afford 4′-cyclopropyl-44({4-[1-isopropyl-4-(trifluoromethyl)imidazol-2-yl]phenyl}methyl)amino]-6′-methoxy-[2,5′-bipyrimidine]-5-carbonitrile (422.4 mg, 75.92%) as a light yellow solid. LC-MS (ESI) m/z 535.5 [M+H]

### Step 3 5-(Aminomethyl)-4′-Cyclopropyl-N-({4-[4-(Difluoromethyl)-1-Isopropylimidazol-2-Yl]Phenyl}Methyl)-6′-Methoxy-12,5′-Bipyrimidin]-4-Amine

To a stirred solution of 4′-cyclopropyl-44({4-[1-isopropyl-4-(trifluoromethyl)imidazol-2-yl]phenyl}methyl)amino]-6′-methoxy-[2,5′-bipyrimidine]-5-carbonitrile (422.4 mg, 0.790 mmol, 1 equiv) in THF (5 mL) was added LiAlH₄ (89.96 mg, 2.370 mmol, 3 equiv) at 0° C. under nitrogen atmosphere. The resulting mixture was stirred for 1 h at 0° C. under nitrogen atmosphere. The reaction was quenched with water at room temperature. The resulting mixture was extracted with EA (3×10 mL), dried over anhydrous Na₂SO₄ and concentrated under reduced pressure. The residue was purified by reverse flash chromatography (C18 silica gel column, eluting with 10% to 50% MeCN/water (0.1% FA) in 10 min with UV detector (254 nm)) to afford 5-(aminomethyl)-4′-cyclopropyl-N-({4-[4-(difluoromethyl)-1-isopropylimidazol-2-yl]phenyl}methyl)-6′-methoxy-[2,5′-bipyrimidin]-4-amine (161.5 mg, 39.26%) as a colorless oil. LC-MS (ESI) m/z 539.5 [M+H]

### Step 4: 7-(4-Cyclopropyl-6-Methoxypyrimidin-5-Yl)-1-({4-[1-Isopropyl-4-(Trifluoromethyl)Imidazol-2-Yl]Phenyl}Methyl)-311,411-Pyrimido[4,5-d][1,3]Diazin-2-One

To a stirred solution of 5-(aminomethyl)-4′-cyclopropyl-N-({4-[1-isopropyl-4-(trifluoromethyl)imidazo]-2-yl]phenyl}methyl)-6′-methoxy-[2,5′-bipyrimidin]-4-amine (187 mg, mmol, 1 equiv) in DMF (2 mL) was added NaH (16.66 mg, 0.694 mmol, 2 equiv) at room temperature under nitrogen atmosphere. The resulting mixture was stirred for 5 min at room temperature under nitrogen atmosphere. To the above mixture was added CDI (112.60 mg, 0.694 mmol, 2 equiv) at room temperature. The resulting mixture was stirred for additional overnight at room temperature. The reaction was quenched with water at room temperature. The resulting mixture was extracted with EA (3×10 mL), dried over anhydrous Na₂SO₄ and concentrated under reduced pressure. The residue was purified by reverse flash chromatography (C18 silica gel column, eluting with 0% to 100% MeCN/water (0.1% FA) in 10 min with UV detector (254 nm)) to afford 7-(4-cyclopropyl-6-methoxypyrimidin-5-yl)-14 {441-isopropyl-4-(trifluoromethyl)imidazol-2-yl]phenyl}methyl)-3H,4H-pyrimido[4,5-d][1,3]diazin-2-one (11.7 mg, 5.89%) as an off-white solid. ¹H NMR: (400 MHz, DMSO-d6) δ 8.70 (s, 1H), 8.62 (s, 1H), 8.23 (s, 1H), 7.78 (s, 1H), 7.54 (d, J=7.9 Hz, 2H), 7.46 (d, J=7.9 Hz, 2H), 5.24 (s, 2H), 4.60 (s, 2H), 4.56-4.45 (m, 1H), 3.87 (s, 3H), 1.71 (s, 1H), 1.46 (d, J=6.6 Hz, 6H), 1.02 (d, J=4.5 Hz, 2H), 0.79 (dd, J=8.0, 3.8 Hz, 2H). LC-MS (ESI) m/z 565.10 [M+H]1-25

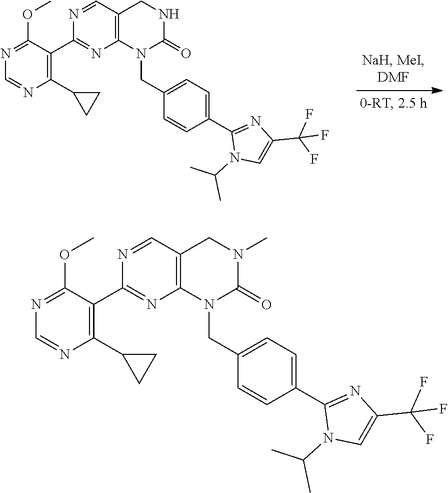

To a stirred solution of 7-(4-cyclopropyl-6-methoxypyrimidin-5-yl)-1-({4-[1-isopropyl-4-(trifluoromethyl)imidazo]-2-yl]phenyl}methyl)-3H,4H-pyrimido[4,5-d][1,3]diazin-2-one (16.7 mg, 0.030 mmol, 1 equiv) in DMF (2 mL) was added NaH (1.42 mg, 0.060 mmol, 2 equiv) at 0° C. under nitrogen atmosphere. The resulting mixture was stirred for 30 min at 0° C. under nitrogen atmosphere. To the above mixture was added MeI (12.60 mg, 0.090 mmol, 3 equiv) dropwise at 0° C. The resulting mixture was stirred for additional 2 h at room temperature. The reaction was quenched with water at room temperature. The resulting mixture was extracted with EA (2×10 mL), dried over anhydrous Na₂SO₄, filtered and concentrated under reduced pressure. The residue was purified by reverse flash chromatography (C18 silica gel column, eluting with 10% to 50% MeCN/water (0.1% FA) in 10 min with UV detector (254 nm)) to afford 7-(4-cyclopropyl-6-methoxypyrimidin-5-yl)-1-({4-[1-isopropyl-4-(trifluoromethyl)imidazol-2-yl]phenyl methyl)-3-methyl-4H-pyrimido[4,5-d][1,3]diazin-2-one (7.1 mg, 37.71%) as a off-white solid. ¹H NMR (400 MHz, DMSO-d6) δ 8.60 (d, J=25.0 Hz, 2H), 8.15 (s, 1H), 7.52-7.34 (m, 4H), 5.20 (s, 2H), 4.62 (s, 2H), 4.43 (p, J=6.8 Hz, 1H), 3.80 (s, 3H), 2.99 (s, 3H), 1.64 (p, J=7.5, 5.9 Hz, 1H), 1.39 (d, J=6.6 Hz, 6H), 0.96 (s, 2H), 0.78-0.67 (m, 2H). LC-MS (ESI) m/z 579.05 [M+H]

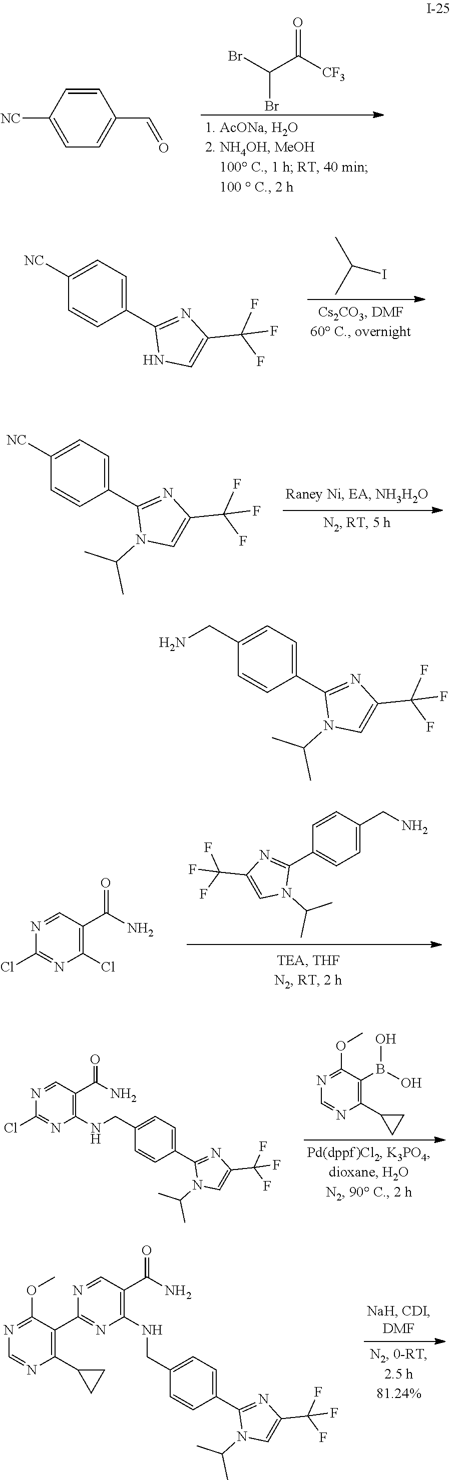

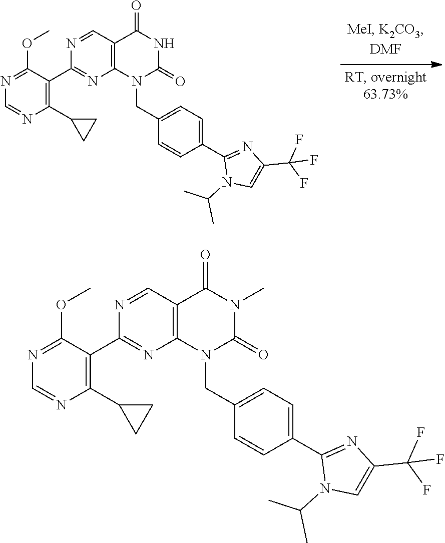

### Step 1: 4-[4-(Trifluoromethyl)-1H-Imidazol-2-Yl]Benzonitrile

To a stirred solution of 3,3-dibromo-1,1,1-trifluoropropan-2-one (23.05 g, 85.409 mmol, 1.12 equiv) in H₂O (20 mL) was added AcONa (7.01 g, 85.409 mmol, 1.12 equiv) at room temperature under nitrogen atmosphere. The resulting mixture was stirred for 1 h at 100° C. under nitrogen atmosphere, then cooled to room temperature. To the above mixture was added benzonitrile, 4-formyl-(10 g, 76.258 mmol, 1 equiv) in MeOH (152 mL) and NH₃H₂O (35.6 mL). The resulting mixture was stirred for 40 min at room temperature. Then the reaction was warmed to 100° C. and stirred for 2 h. The reaction was quenched by the addition of Water/Ice (50 mL) at room temperature. The resulting mixture was extracted with EA (2×60 mL), dried over anhydrous Na₂SO₄ and concentrated under reduced pressure. The residue was purified by silica gel column chromatography, (eluting with PE/EA (5:1)) to afford 4-[4-(trifluoromethyl)-1H-imidazol-2-yl]benzonitrile (13.8 g, 76.30%) as a yellow solid. LC-MS (ESI) m/z 238.1 [M+H]

### Step 2: 4-[1-Isopropyl-4-(Trifluoromethyl)Imidazol-2-Yl]Benzonitrile

To a stirred solution of 4-[4-(trifluoromethyl)-1H-imidazol-2-yl]benzonitrile (5 g, 21.081 mmol, 1 equiv) in DMF (80 mL) was added Cs₂CO₃ (13.74 g, 42.162 mmol, 2 equiv) at room temperature under nitrogen atmosphere. The resulting mixture was stirred for 2 min at room temperature under nitrogen atmosphere. To the above mixture was added 2-iodopropane (10.75 g, 63.243 mmol, 3 equiv) dropwise at room temperature. The resulting mixture was stirred for additional overnight at 60° C. The reaction was quenched with water at room temperature. The resulting mixture was extracted with EA (3×60 mL). The combined organic layers were washed with brine (3×60 mL), dried over anhydrous Na₂SO₄ and concentrated under reduced pressure. The residue was purified by silica gel column chromatography(eluting with PE/EA (5:1)) to afford 4-[1-isopropyl-4-(trifluoromethyl)imidazol-2-yl]benzonitrile (3.3 g, 56.05%) as a yellow solid. LC-MS (ESI) m/z 280.2 [M+H]

### Step 3: 1-{4-[1-Isopropyl-4-(Trifluoromethyl)Imidazol-2-Yl]Phenyl}Methanamine

To a stirred solution of 4-[1-isopropyl-4-(trifluoromethyl)imidazol-2-yl]benzonitrile (3.5 g, 12.533 mmol, 1 equiv) in EA (40 mL) and NH₃H₂O (20 mL) was added Raney Ni (3.96 g, 46.264 mmol, 3.4 equiv) at room temperature under air atmosphere. The resulting mixture was stirred for 5 h at room temperature under hydrogen atmosphere. The resulting mixture was filtered, the filter cake was washed with EA (3×20 mL) and concentrated under reduced pressure. The residue was purified by reverse flash chromatography (C18 silica gel column, eluting with 0% to 100% MeCN/water (0.1% FA) in 10 min with UV detector (254 nm)) to afford 1-{4-[1-isopropyl-4-(trifluoromethyl)imidazol-2-yl]phenyl}methanamine (3.3 g, as an off-white solid. ¹H NMR (400 MHz, DMSO-d6) δ 8.44 (s, 1H), 8.19 (d, J=1.4 Hz, 1H), 7.59 (s, 5H), 4.47 (p, J=6.6 Hz, 1H), 4.02 (s, 2H), 1.41 (d, J=6.7 Hz, 6H). LC-MS (ESI) m/z 284.2 [M+H]

### Step 4: 2-Chloro-4-l({4-[1-Isopropyl-4-(Trifluoromethyl)Imidazol-2-Yl]Phenyl}Methyl)Aminol Pyrimidine-5-Carboxamide

To a stirred solution of 2,4-dichloropyrimidine-5-carboxamide (638 mg, 3.323 mmol, 1 equiv) and 1-{4-[1-isopropyl-4-(trifluoromethyl)imidazo]-2-yl]phenyl}methanamine (941.38 mg, 3.323 mmol, 1 equiv) in THF (5 mL) was added TEA (672.51 mg, 6.646 mmol, 2 equiv) dropwise at room temperature under nitrogen atmosphere. The resulting mixture was stirred for 2 h at room temperature under nitrogen atmosphere. The residue was purified by reverse flash chromatography (C18 silica gel column, eluting with 0% to 100% MeCN/water (0.1% FA) in 10 min with UV detector (254 nm)) to afford 2-chloro-4-[({4-[1-isopropyl-4-(trifluoromethyl)imidazol-2-yl]phenyl methyl)amino]pyrimidine-5-carboxamide (780 mg, 53.49%) as an off-white solid. LC-MS (ESI) m/z 439.8 [M+H]

### Step 5: 4′-Cyclopropyl-4-1({4-[1-Isopropyl-4-(Trifluoromethyl)Imidazol-2-Yl]Phenyl}Methyl)Amino]-6′-Methoxy-12,5′-Bipyrimidine]-5-Carboxamide

To a stirred solution of 2-chloro-44({4-[1-isopropyl-4-(trifluoromethyl)imidazo]-2-yl]phenyl}methyl)amino]pyrimidine-5-carboxamide (400 mg, 0.911 mmol, 1 equiv) and 4-cyclopropyl-6-methoxypyrimidin-5-ylboronic acid (265.24 mg, 1.367 mmol, 1.5 equiv) in dioxane (4 mL) and H₂O (0.5 mL) were added K₃PO₄ (483.70 mg, 2.277 mmol, 2.5 equiv) and Pd(dppf)C12 (133.39 mg, 0.182 mmol, 0.2 equiv) at room temperature under nitrogen atmosphere. The resulting mixture was stirred for 2 h at 90° C. under nitrogen atmosphere. The residue was purified by reverse flash chromatography (C18 silica gel column, eluting with 10% to 50% MeCN/water (0.1% FA) in 10 min with UV detector (254 nm)) to afford 4′-cyclopropyl-44({4-[1-isopropyl-4-(trifluoromethyl)imidazo]-2-yl]phenyl methyl)amino]-6′-methoxy-[2,5′-bipyrimidine]-5-carboxamide (485 mg, 96.30%) as a light yellow solid. LC-MS (ESI) m/z 553.5 [M+H]

### Step 6: 7-(4-Cyclopropyl-6-Methoxypyrimidin-5-Yl)-1-({4-[1-Isopropyl-4-(Trifluoromethyl)Imidazol-2-Yl]Phenyl}Methyl)-311-Pyrimido[4,5-d][1,3]Diazine-2,4-Dione

To a stirred solution of 4′-cyclopropyl-44({4-[1-isopropyl-4-(trifluoromethyl)imidazo]-2-yl]phenyl methyl)amino]-6′-methoxy-[2,5′-bipyrimidine]-5-carboxamide (435 mg, 0.787 mmol, 1 equiv) in DMF (5 mL) was added NaH (37.78 mg, 1.574 mmol, 2 equiv) at 0° C. under nitrogen atmosphere. The resulting mixture was stirred for 5 min at room temperature under nitrogen atmosphere. To the above mixture was added CDI (255.31 mg, 1.574 mmol, 2 equiv) at room temperature. The resulting mixture was stirred for additional 2 h at room temperature. The reaction was quenched with Water at room temperature. The resulting mixture was extracted with EA (3×15 mL), dried over anhydrous Na₂SO₄ and concentrated under reduced pressure. The residue was purified by reverse flash chromatography (C18 silica gel column, eluting with 10% to 50% MeCN/water (0.1% FA) in 10 min with UV detector (254 nm)) to afford 7-(4-cyclopropyl-6-methoxypyrimidin-5-yl)-14 {441-isopropyl-4-(trifluoromethyl)imidazol-2-yl]phenyl}methyl)-3H-pyrimido[4,5-d][1,3]diazine-2,4-dione (370 mg, 81.24%) as an off-white solid. LC-MS (ESI) m/z 579.5 [M+H]

### Step 7: 7-(4-Cyclopropyl-6-Methoxypyrimidin-5-Yl)-1-({4-[1-Isopropyl-4-(Trifluoromethyl)Imidazol-2-Yl]Phenyl}Methyl)-3-Methylpyrimido[4,5-d][1,3]Diazine-2,4-Dione

To a stirred solution of 7-(4-cyclopropyl-6-methoxypyrimidin-5-yl)-1-({4-[1-isopropyl-4-(trifluoromethyl)imidazol-2-yl]phenyl}methyl)-3H-pyrimido[4,5-d][1,3]diazine-2,4-dione (30 mg, 0.052 mmol, 1 equiv) in DMF (1 mL) were added K₂CO₃ (14.33 mg, 0.104 mmol, 2 equiv) and Mel (14.72 mg, 0.104 mmol, 2 equiv) at room temperature under nitrogen atmosphere. The resulting mixture was stirred for overnight at room temperature under nitrogen atmosphere. The residue was purified by reverse flash chromatography (C18 silica gel column, eluting with 10% to 50% MeCN/water (0.1% FA) in 10 min with UV detector (254 nm)) to afford 7-(4-cyclopropyl-6-methoxypyrimidin-5-yl)-1-({4-[1-isopropyl-4-(trifluoromethyl)imidazol-2-yl]phenyl}methyl)-3-methylpyrimido[4,5-d][1,3]diazine-2,4-dione (19.7 mg, 63.73%) as an off-white solid. ¹H NMR: (400 MHz, DMSO-d6) δ 9.37 (s, 1H), 8.69 (s, 1H), 8.17 (s, 1H), 7.50 (t, J=6.4 Hz, 4H), 5.44 (s, 2H), 4.42 (p, J=6.7 Hz, 1H), 3.83 (s, 3H), 3.34 (s, 3H), 1.72 (tt, J=8.5, 4.7 Hz, 1H), 1.39 (d, J=6.6 Hz, 6H), 1.01 (p, J=3.5 Hz, 2H), 0.75 (dq, J=7.3, 3.6 Hz, 2H). LC-MS (ESI) m/z 593.10 [M+H].

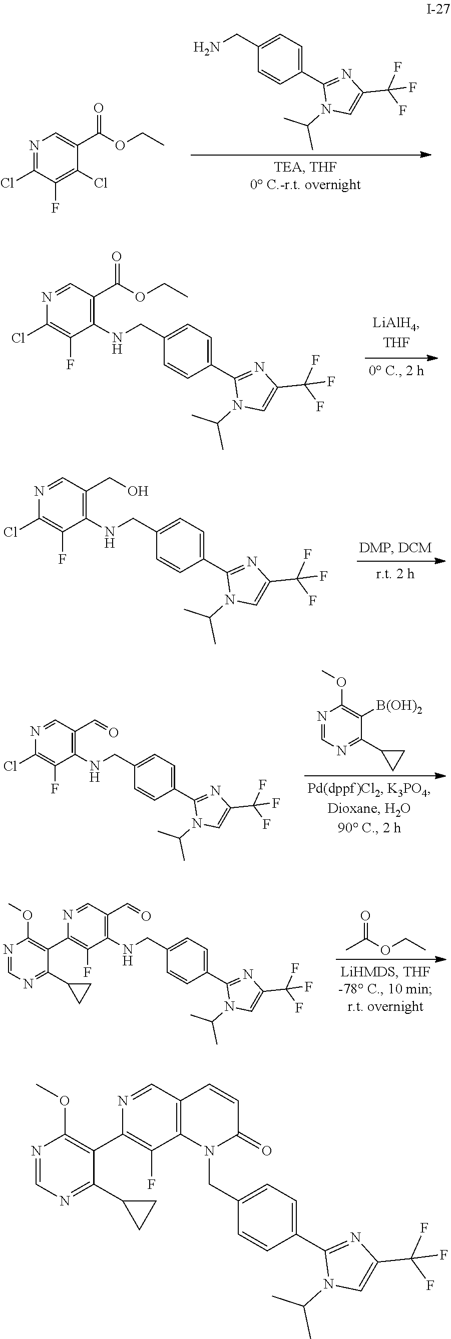

### Step 1: Ethyl 6-Chloro-5-Fluoro-4-1({4-[1-Isopropyl-4-(Trifluoromethyl)Imidazol-2-Yl]Phenyl}Methyl)Amino]Pyridine-3-Carboxylate

To a stirred solution of ethyl 4,6-dichloro-5-fluoropyridine-3-carboxylate (900 mg, 3.781 mmol, 1 equiv) and TEA (1147.79 mg, 11.343 mmol, 3 equiv) in THF (10 mL) was added 1-{4-[1-isopropyl-4-(trifluoromethyl)imidazol-2-yl]phenyl methanamine (1071.11 mg, 3.781 mmol, 1 equiv) dropwise at 0° C. The resulting mixture was stirred for overnight at room temperature and concentrated under reduced pressure. The residue was purified by reverse flash chromatography (C18 silica gel column, eluting with 0% to 100% MeCN/water (0.1% FA) in 10 min with UV detector (254 nm)) to afford ethyl 6-chloro-5-fluoro-44({4-[1-isopropyl-4-(trifluoromethyl)imidazol-2-yl]phenyl}methyl)amino]pyridine-3-carboxylate (450 mg, 24.55%) as a yellow solid. LC-MS (ESI) m/z 485.8 [M+H].

### Step 2: {6-Chloro-5-Fluoro-4-1({4-[1-Isopropyl-4-(Trifluoromethyl)Imidazol-2-Yl]Phenyl}Methyl)Amino]Pyridin-3-Yl}Methanol

To a stirred solution of ethyl 6-chloro-5-fluoro-4-[({4-[1-isopropyl-4-(trifluoromethyl)imidazo]-2-yl]phenyl}methyl)amino]pyridine-3-carboxylate (400 mg, 0.825 mmol, 1 equiv) in THF (5 mL) was added LiAlH₄ (40.70 mg, 1.073 mmol, 1.3 equiv) in portions at 0° C. The resulting mixture was stirred for 2 h at 0° C. under nitrogen atmosphere. The resulting mixture was filtered, and the filter cake was washed with THF (3×10 mL). The filtrate was concentrated under reduced pressure. The residue was purified by reverse flash chromatography (C18 silica gel column, eluting with 10% to 50% MeCN/water (0.1% FA) in 10 min with UV detector (254 nm)) to afford {6-chloro-5-fluoro-4-[({4-[1-isopropyl-4-(trifluoromethyl)imidazol-2-yl]phenyl methyl)amino]pyri din-3-yl}methanol (200 mg, 54.7%) as a brown solid. LC-MS (ESI) m/z 443.8 [M+H].

### Step 3: 6-Chloro-5-Fluoro-4-1({4-[1-Isopropyl-4-(Trifluoromethyl)Imidazol-2-Yl]Phenyl}Methyl)Amino]Pyridine-3-Carbaldehyde

To a stirred solution of {6-chloro-5-fluoro-44({4-[1-isopropyl-4-(trifluoromethyl)imidazo]-2-yl]phenyl methyl)amino]pyridin-3-yl}methanol (200 mg, 0.452 mmol, 1 equiv) in DCM (2 mL) was added DMP (383.11 mg, 0.904 mmol, 2 equiv) at room temperature. The resulting mixture was stirred for 2 h at room temperature, and concentrated under reduced pressure. The residue was purified by reverse flash chromatography (C18 silica gel column, eluting with 10% to 100% MeCN/water (0.1% FA) in 10 min with UV detector (254 nm)) to 6-chloro-5-fluoro-44({4-[1-isopropyl-4-(trifluoromethyl)imidazol-2-yl]phenyl methyl)amino]pyridine-3-carb aldehyde (150 mg, 75.34%) as a light yellow solid. LC-MS (ESI) m/z: 441.1 [M+H]

### Step 4: 6-(4-Cyclopropyl-6-Methoxypyrimidin-5-Yl)-5-Fluoro-4-1({4-[1-Isopropyl-4-(Trifluoromethyl)Imidazol-2-Yl]Phenyl}Methyl)Amino]Pyridine-3-Carbaldehyde

To a stirred solution of 6-chloro-5-fluoro-4-[({4-[1-isopropyl-4-(trifluoromethyl)imidazol-2-yl]phenyl}methyl)amino]pyridine-3-carbaldehyde (50 mg, 0.113 mmol, 1 equiv) and 4-cyclopropyl-6-methoxypyrimidin-5-ylboronic acid (33.01 mg, 0.170 mmol, 1.5 equiv) in 1,4-dioxane (1 mL) and H₂O (0.2 mL) was added Pd(dppf)C12 (16.60 mg, 0.023 mmol, 0.2 equiv) and K₃PO₄ (60.19 mg, 0.283 mmol, 2.5 equiv) at room temperature. The resulting mixture was stirred for 2 h at 90° C. under nitrogen atmosphere. The resulting mixture was filtered, and the filter cake was washed with 1,4-dioxane (3×10 mL). The filtrate was concentrated under reduced pressure. The residue was purified by reverse flash chromatography (C18 silica gel column, eluting with 10% to 50% MeCN/water (0.1% TFA) in 10 min with UV detector (254 nm)) to afford 6-(4-cyclopropyl-6-methoxypyrimidin-5-yl)-5-fluoro-44({4-[1-isopropyl-4-(trifluoromethyl)imidazol-2-yl]phenyl}methyl)amino]pyridine-3-carbaldehyde (35 mg, 55.6%) as an off-white solid. LC-MS (ESI) m/z 555.5 [M+H].

### Step 5: 7-(4-Cyclopropyl-6-Methoxypyrimidin-5-Yl)-8-Fluoro-1-({4-[1-Isopropyl-4-(Trifluoromethyl)Imidazol-2-Yl]Phenyl}Methyl)-1,6-Naphthyridin-2-One

To a stirred solution of ethyl acetate (79.44 mg, 0.900 mmol, 10 equiv) in THF (2 mL) was added LiHMDS (0.90 mL, 0.900 mmol, 10 equiv,. 1M in THF) dropwise at −78° C. under nitrogen atmosphere. The resulting mixture was stirred for 10 min at −78° C. under nitrogen atmosphere. Next, the solution of 6-(4-cyclopropyl-6-methoxypyrimidin-5-yl)-5-fluoro-44({4-[1-isopropyl-4-(trifluoromethyl)imidazo]-2-yl]phenyl methyl)amino]pyridine-3-carb aldehyde (50 mg, 0.090 mmol, 1 equiv) in THF(1 mL) was added to the reaction mixture at −78° C. The resulting mixture was stirred overnight at room temperature under nitrogen atmosphere and concentrated under reduced pressure. The residue was purified by reverse flash chromatography (C18 silica gel column, eluting with 10% to 100% MeCN/water (0.1% FA) in 15 min with UV detector (254 nm)) to afford 7-(4-cyclopropyl-6-methoxypyrimidin-5-yl)-8-fluoro-1-({4-[1-isopropyl-4-(trifluoromethyl)imidazol-2-yl]phenyl}methyl)-1,6-naphthyridin-2-one (16.6 mg, 31.6%) as an off-white solid. ¹H NMR (400 MHz, DMSO-d6) δ 8.92 (s, 1H), 8.66 (s, 1H), 8.26-8.19 (m, 1H), 8.15 (s, 1H), 7.51 (d, J=8.0 Hz, 2H), 7.27 (d, J=8.0 Hz, 2H), 6.96 (d, J=9.5 Hz, 1H), 5.69-(m, 2H), 4.43 (p, J=6.6 Hz, 1H), 3.73 (s, 3H), 1.60 (s, 1H), 1.38 (d, J=6.6 Hz, 6H), 1.11-1.03 (m, 1H), 0.92-0.81 (m, 2H), 0.70 (s, 1H). LC-MS (ESI) m/z 579.1 [M+H]

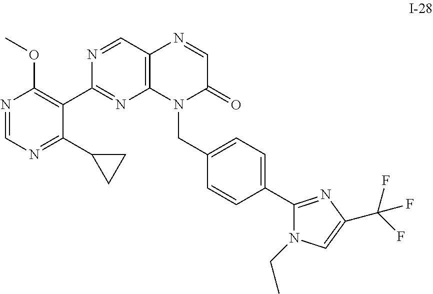 [SMILES: CCn1cc(C(F)(F)F)nc1-c1ccc(Cn2c(=O)cnc3cnc(-c4c(OC)ncnc4C4CC4)nc32)cc1]

1-28 was made in an analogous way as 1-17. ¹H NMR (400 MHz, DMSO-d6) δ 9.38 (d, J=1.1 Hz, 1H), 8.70 (s, 1H), 8.48 (s, 1H), 8.02 (s, 1H), 7.56 (d, J=7.9 Hz, 2H), 7.49 (d, J=8.0 Hz, 2H), 5.49 (s, 2H), 4.04 (q, J=7.2 Hz, 2H), 3.91-3.80 (m, 3H), 1.76 (s, 1H), 1.29 (t, J=7.2 Hz, 3H), 1.03 (s, 2H), 0.78 (s, 2H). LC-MS (ESI) m/z 549.0 [M+H]

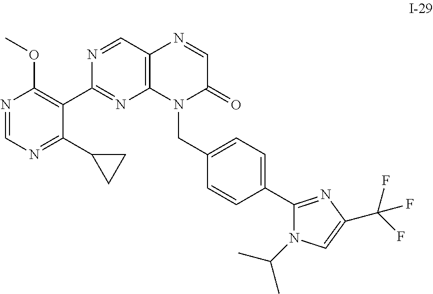 [SMILES: COc1ncnc(C2CC2)c1-c1ncc2ncc(=O)n(Cc3ccc(-c4nc(C(F)(F)F)cn4C(C)C)cc3)c2n1]

1-29 was made in an analogous way as 1-17. ¹H NMR (300 MHz, DMSO-d6) δ 9.38 (s, 1H), 8.71 (s, 1H), 8.49 (s, 1H), 8.18 (d, J=1.4 Hz, 1H), 7.50 (s, 4H), 5.51 (s, 2H), 4.41 (p, J=6.7 Hz, 1H), 3.85 (s, 3H), 1.75 (m, 1H), 1.39 (d, J=6.6 Hz, 6H), 1.10-0.99 (m, 2H), 0.82-0.70 (m, 2H). LC-MS (ESI) m/z 563.1 [M+H].

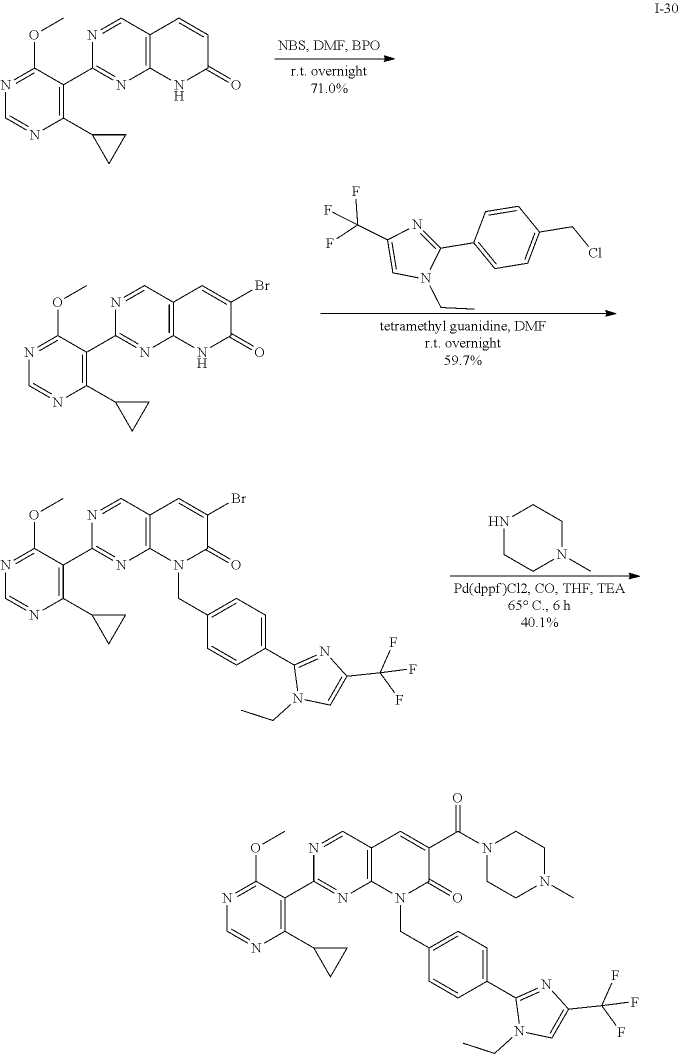

### Step 1: 6-Bromo-2-(4-Cyclopropyl-6-Methoxypyrimidin-5-Yl)-811-Pyrido[2,3-d]Pyrimidin-7-One

To a stirred solution of 2-(4-cyclopropyl-6-methoxypyrimidin-5-yl)-8H-pyrido[2,3-d]pyrimidin-7-one (1 g, 3.386 mmol, 1 equiv) and NB S (1.81 g, 10.158 mmol, 3 equiv) in DMF (15 mL) was added BPO (0.17 g, 0.677 mmol, 0.2 equiv) at room temperature. The resulting mixture was stirred for overnight at room temperature under nitrogen atmosphere and concentrated under reduced pressure. The residue was purified by reverse flash chromatography (C18 silica gel column, eluting with 0% to 100% MeCN/water (0.1% TFA) in 10 min with UV detector (254 nm)) to afford 6-bromo-2-(4-cyclopropyl-6-methoxypyrimidin-5-yl)-8H-pyrido[2,3-d]pyrimidin-7-one (900 mg, 71.0%) as a yellow solid. LC-MS (ESI) m/z 374.2 [M+H].

### Step 2: 6-Bromo-2-(4-Cyclopropyl-6-Methoxypyrimidin-5-Yl)-8-({4-[1-Ethyl-4-(Trifluoromethyl)Imidazol-2-Yl]Phenyl}Methyl)Pyrido[2,3-d]Pyrimidin-7-One

To a stirred solution of 6-bromo-2-(4-cyclopropyl-6-methoxypyrimidin-5-yl)-8H-pyrido[2,3-d]pyrimidin-7-one (150 mg, 0.401 mmol, 1 equiv) and 2-[4-(chloromethyl)phenyl]-1-ethyl-4-(trifluoromethyl)imidazole (104.15 mg, 0.361 mmol, 0.9 equiv) in DMF (2 mL) was added 1,1,3,3-tetramethylguanidine (69.25 mg, 0.602 mmol, 1.5 equiv) dropwise at room temperature. The resulting mixture was stirred for overnight at room temperature under nitrogen atmosphere. The residue was purified by reverse flash chromatography (C18 silica gel column, eluting with 0% to 100% MeCN/water (0.1% FA) in 10 min with UV detector (254 nm)) to afford 6-bromo-2-(4-cyclopropyl-6-methoxypyrimidin-5-yl)-8-({4-[1-ethyl-4-(trifluoromethyl)imidazo]-2-yl]phenyl}methyl)pyrido[2,3-d]pyrimidin-7-one (150 mg, 59.7%) as a yellow solid. LC-MS (ESI) m/z 626.4 [M+H].

### Step 3: 2-(4-Cyclopropyl-6-Methoxypyrimidin-5-Yl)-8-({4-[1-Ethyl-4-(Trifluoromethyl)Imidazol-2-Yl]Phenyl}Methyl)-6-(4-Methylpiperazine-1-Carbonyl)Pyrido[2,3-d]Pyrimidin-7-One

To a stirred solution of 6-bromo-2-(4-cyclopropyl-6-methoxypyrimidin-5-yl)-8-({4-[1-ethyl-4-(trifluoromethyl)imidazo]-2-yl]phenyl}methyl)pyrido[2,3-d]pyrimidin-7-one (50 mg, 0.080 mmol, 1 equiv) and piperazine, 1-methyl-(31.98 mg, 0.320 mmol, 4 equiv) in THF (4 mL) was added TEA (40.38 mg, 0.400 mmol, 5 equiv) and Pd(dppf)Cl₂ (11.68 mg, 0.016 mmol, equiv) at room temperature. The resulting mixture was stirred for 6 h at 65° C. under carbon monoxide atmosphere. The resulting mixture was filtered, the filter cake was washed with 1,4-dioxane (3×20 mL). The filtrate was concentrated under reduced pressure. The crude product was purified by Prep-HPLC with the following conditions (Column: YMC-Actus Triart C18, 30*150 mm, 51 μm; Mobile Phase A: Water(0.05% TFA), Mobile Phase B: ACN; Flow rate: 60 mL/min; Gradient: 25% B to 45% B in 7 min, 45% B; Wave Length: 254/220 nm; RT1(min): 6.18) to afford 2-(4-cyclopropyl-6-methoxypyrimidin-5-yl)-8-({4-[1-ethyl-4-(trifluoromethyl)imidazo]-2-yl]phenyl}methyl)-6-(4-methylpiperazine-1-carbonyl)pyrido[2,3-d]pyrimidin-7-one (22.3 mg, as an off-white solid. ¹H NMR (400 MHz, DMSO-d6) δ 9.36 (s, 1H), 8.71 (s, 1H), 8.30 (s, 1H), 8.02 (d, J=1.4 Hz, 1H), 7.55 (d, J=8.2 Hz, 2H), 7.44 (d, J=8.1 Hz, 2H), 5.60 (s, 2H), 4.56 (s, 1H), 4.05 (q, J=7.2 Hz, 2H), 3.84 (s, 4H), 3.54 (s, 1H), 3.37 (s, 2H), 3.10 (d, J=42.9 Hz, 3H), 2.86 (s, 3H), 1.72 (m, 1H), 1.29 (t, J=7.3 Hz, 3H), 1.03 (t, J=4.0 Hz, 2H), 0.77 (m, 2H). LC-MS (ESI) m/z: 674.3 [M+H]

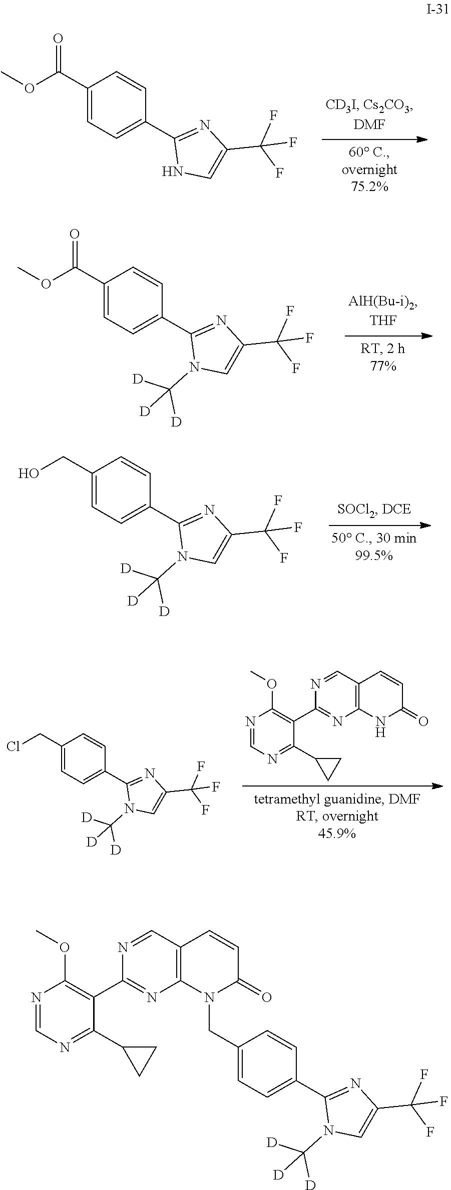 [SMILES: COC(=O)c1ccc(-c2nc(C(F)(F)F)c[nH]2)cc1 ; [2H]C([2H])([2H])n1cc(C(F)(F)F)nc1-c1ccc(C(=O)OC)cc1 ; [2H]C([2H])([2H])n1cc(C(F)(F)F)nc1-c1ccc(CO)cc1 ; [2H]C([2H])([2H])n1cc(C(F)(F)F)nc1-c1ccc(CCl)cc1 ; COc1ncnc(C2CC2)c1-c1ncc2ccc(=O)[nH]c2n1 ; [2H]C([2H])([2H])n1cc(C(F)(F)F)nc1-c1ccc(Cn2c(=O)ccc3cnc(-c4c(OC)ncnc4C4CC4)nc32)cc1]

### Step 1: 4-[1-(2113)Methyl-4-(Trifluoromethyl)Imidazol-2-Yl]Benzoate

To a stirred mixture of methyl 4-[4-(trifluoromethyl)-1H-imidazol-2-yl]benzoate (2 g, 7.402 mmol, 1 equiv) and Cs₂CO₃ (4.82 g, 14.804 mmol, 2 equiv) in DMF (40 mL, 516.862 mmol, 69.83 equiv) was added CD 3 I (1.07 g, 7.402 mmol, 1 equiv) dropwise at room temperature under nitrogen atmosphere. The resulting mixture was stirred for overnight at under nitrogen atmosphere. The resulting mixture was extracted with EtOAc (3×100 mL). The combined organic layers were washed with brine (3×10 mL), dried over anhydro us Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford methyl 4-[1-(2H3)methyl-4-(trifluoromethyl)imidazol-2-yl]benzoate (1.6 g, 75.25%) as a yellow solid. LC-MS (ESI) m/z 288.1 [M+H]

### Step 2: {4-[1-(2H3)Methyl-4-(Trifluoromethyl)Imidazol-2-Yl]Phenyl}Methanol

To a stirred mixture of methyl 4-[1-(2H3)methyl-4-(trifluoromethyl)imidazol-2-yl]benzoate (2.3 g, 8.007 mmol, 1 equiv) in THF (45 mL, 555.425 mmol, 69.37 equiv) was added A1H(Bu-i) 2 (24.02 mL, 24.021 mmol, 3 equiv) dropwise at 0° C. under nitrogen atmosphere. The resulting mixture was stirred for 2 h at room temperature under nitrogen atmosphere. The reaction was quenched with sat. NH₄Cl (aq.) at 0° C. The resulting mixture was extracted with EtOAc (3×100 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford {4-[1-(2H3)methyl-4-(trifluoromethyl)imidazol-2-yl]phenyl}methanol (1.6 g, 77.08%) as an off-white solid. LC-MS (ESI) m/z 260.1 [M+H]

### Step 3: 2-[4-(Chloromethyl)Phenyl]-1-(2H3)Methyl-4-(Trifluoromethyl)Imidazole

To a stirred mixture of {4-[1-(2H3)methyl-4-(trifluoromethyl)imidazol-2-yl]phenyl}methanol (1.5 g, 5.786 mmol, 1 equiv) in DCE (30 mL, 378.979 mmol, 65.50 equiv) was added thionyl chloride (2.06 g, 17.358 mmol, 3 equiv) dropwise at room temperature under nitrogen atmosphere. The resulting mixture was stirred for 30 min at under nitrogen atmosphere. The resulting mixture was extracted with EtOAc (3×100 mL). The combined organic layers were washed with brine (3×10 mL), dried over anhydrous Na₂SO₄, filtered and concentrated under reduced pressure to afford 2-[4-(chloromethyl)phenyl]-1-(2H3)methyl-4-(trifluoromethyl)imidazole (1.6 g, 99.58%) as an off-white solid. LC-MS(ESI) m/z 254.06 [M+H]

### Step 4: 2-(4-Cyclopropyl-6-Methoxypyrimidin-5-Yl)-8-({4-[1-(2H3)Methyl-4-(Trifluoromethyl)Imidazol-2-Yl]Phenyl}Methyl)Pyrido[2,3-d]Pyrimidin-7-One

A mixture of 2-[4-(chloromethyl)phenyl]-1-(2H3)methyl-4-(trifluoromethyl)imidazole (36.39 mg, 0.131 mmol, 0.9 equiv),1,1,3,3-tetramethylguanidine (25.16 mg, 0.219 mmol, 1.5 equiv) and 2-(4-cyclopropyl-6-methoxypyrimidin-5-yl)-8H-pyrido[2,3-d]pyrimidin-7-one (43 mg, 0.146 mmol, 1.00 equiv) in DMF (1 mL, 12.922 mmol, 88.74 equiv) was stirred for overnight at room temperature under nitrogen atmosphere. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[1-(2H3)methyl-4-(trifluoromethyl)imidazol-2-yl]phenyl methyl)pyrido[2,3-d]pyrimidin-7-one (36.3 mg, 45.91%) as an off-white solid.′H NMR (400 MHz, DMSO-d6) δ 9.29 (s, 1H), 8.69 (s, 1H), 8.16 (d, J=9.6 Hz, 1H), 7.91 (d, J=1.4 Hz, 1H), 7.66-7.58 (m, 2H), 7.40 (d, J=8.1 Hz, 2H), 6.90 (d, J=9.5 Hz, 1H), 5.57 (s, 2H), 3.83 (s, 3H), 1.73 (dq, J=8.1, 4.1, 3.6 Hz, 1H), 1.01 (p, J=3.8 Hz, 2H), 0.76 (dq, J=6.9, 3.5 Hz, 2H). LC-MS (ESI) m/z 537.2 [M+H]

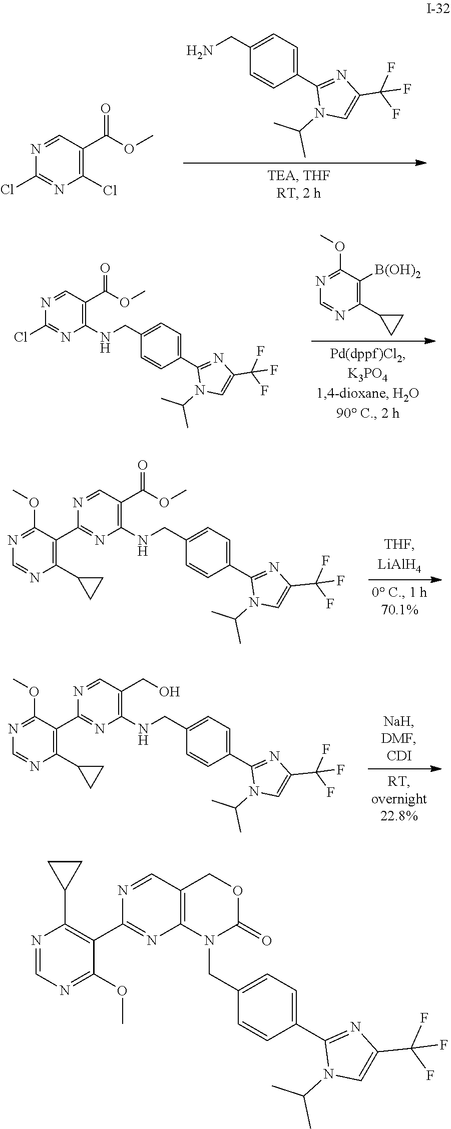

### Step 1 2-Chloro-4-1({4-[1-Isopropyl-4-(Trifluoromethyl)Imidazol-2-Yl]Phenyl}Methyl)Amino]Pyrimidine-5-Carboxylate

To a stirred mixture of methyl 2,4-dichloropyrimidine-5-carboxylate (900 mg, 4.348 mmol, 1 equiv) and 1-{4-[1-isopropyl-4-(trifluoromethyl)imidazo]-2-yl]phenyl}methanamine (1231.67 mg, 4.348 mmol, 1.0 equiv) in THF (10 mL) was added TEA (1319.84 mg, 13.044 mmol, 3.0 equiv) at room temperature under nitrogen atmosphere. The resulting mixture was stirred for 2 h at room temperature under nitrogen atmosphere. The resulting mixture was extracted with EtOAc (3×10 mL). The combined organic layers were washed with brine (2×10 mL), dried over anhydrous Na₂SO₄,filtered and concentrated under reduced pressure. The residue was purified by reverse flash chromatography (C18 silica gel column, eluting with 0% to 100% MeCN/water (0.1% FA) in 10 min with UV detector (254 nm)) to afford methyl 2-chloro-44({4-[1-isopropyl-4-(trifluoromethyl)imidazol-2-yl]phenyl}methyl)amino]pyrimidine-5-carboxylate (820 mg, 41.56%) as a white solid. LC-MS (ESI) m/z 454.1 [M+H]

### Step 2: Methyl 4′-Cyclopropyl-44({4-[1-Isopropyl-4-(Trifluoromethyl)Imidazol-2-Yl]Phenyl}Methyl)Amino]-6′-Methoxy-12,5′-Bipyrimidine]-5-Carboxylate

To a stirred mixture of methyl 2-chloro-44({4-[1-isopropyl-4-(trifluoromethyl)imidazol-2-yl]phenyl}methyl)amino]pyrimidine-5-carboxylate (130 mg, 0.286 mmol, 1 equiv) and 4-cyclopropyl-6-methoxypyrimidin-5-ylboronic acid (83.35 mg, 0.429 mmol, 1.5 equiv) in 1,4-dioxane (2.7 mL, 20 equiv) and H₂O (0.3 mL, 20 equiv) were added pd(dppf)C12 (20.96 mg, 0.029 mmol, 0.1 equiv) and K₃PO₄ (182.40 mg, 0.858 mmol, 3.0 equiv) at room temperature under nitrogen atmosphere. The resulting mixture was stirred for 2 h at 90° C. under nitrogen atmosphere. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford methyl 4′-cyclopropyl-4-[({4-[1-isopropyl-4-(trifluoromethyl)imidazol-2-yl]phenyl}methyl)amino]-6′-methoxy-[2,5′-bipyrimidine]-5-carboxylate (150 mg, 92.27%) as a yellow solid. LC-MS (ESI) m/z 568.3 [M+H]

### Step 3: {4′-Cyclopropyl-4-1({4-[1-Isopropyl-4-(Trifluoromethyl)Imidazol-2-Yl]Phenyl}Methyl)Amino]-6′-Methoxy-12,5′-Bipyrimidin]-5-Yl]Methanol

To a stirred methyl 4′-cyclopropyl-44({4-[1-isopropyl-4-(trifluoromethyl)imidazol-2-yl]phenyl}methyl)amino]-6′-methoxy-[2,5′-bipyrimidine]-5-carboxylate (75 mg, 0.132 mmol, 1 equiv) in tetrahydrofuran (1.5 mL, 20 equiv) was added LiAlH₄ (15.04 mg, 0.396 mmol, 3.0 equiv) in portions at 0° C. under nitrogen atmosphere. The resulting mixture was stirred for 1 h at 0° C. under nitrogen atmosphere. The precipitated solids were collected by filtration and washed with THF (3×5 mL). The residue was purified by reverse flash chromatography (C18 silica gel column, eluting with 0% to 100% MeCN/water (0.1% FA) in 10 min with UV detector (254 nm)) to afford {4′-cyclopropyl-44({4-[1-isopropyl-4-(trifluoromethyl)imidazo]-2-yl]phenyl}methyl)amino]-6′-methoxy-[2,5′-bipyrimidin]-5-yl}methanol (50 mg, 70.13%) as a yellow solid. LC-MS (ESI) m/z 540.3 [M+H]

### Step 4: 7-(4-Cyclopropyl-6-Methoxypyrimidin-5-Yl)-1-({4-[1-Isopropyl-4-(Trifluoromethyl)Imidazol-2-Yl]Phenyl}Methyl)-411-Pyrimido[4,5-d][1,3]Oxazin-2-One

To a stirred mixture of {4′-cyclopropyl-44({4-[1-isopropyl-4-(trifluoromethyl)imidazo]-2-yl]phenyl methyl)amino]-6′-methoxy-[2,5′-bipyrimidin]-5-yl}methanol (50 mg, 0.093 mmol, 1 equiv) and NaH (6.67 mg, 0.279 mmol, 3.0 equiv) in DMF (1 mL, 20 equiv) was added CDI (18.03 mg, 0.112 mmol, 1.2 equiv) in portions at room temperature under nitrogen atmosphere. The resulting mixture was stirred for overnight at room temperature under nitrogen atmosphere. The reaction was quenched with water at 0° C. The resulting mixture was concentrated under reduced pressure. The crude product (50 mg) was purified by Prep-HPLC with the following conditions (Column: YMC-Actus Triart C18, 30*150 mm, 51 μm; Mobile Phase A: Water(0.1% FA), Mobile Phase B: ACN; Flow rate: 60 mL/min; Gradient: 47% B to 67% B in 7 min, 67% B; Wave Length: 254/220 nm; RT1(min): 6.53; Number Of Runs: 0) to afford 7-(4-cyclopropyl-6-methoxypyrimidin-5-yl)-1-({4-[1-isopropyl-4-(trifluoromethyl)imidazo]-2-yl]phenyl methyl)-4H-pyrimido[4,5-d][1,3]oxazin-2-one (12 mg, 22.85%) as a white solid. ¹H NMR (400 MHz, DMSO-d6) δ 8.71 (d, J=1.1 Hz, 1H), 8.65 (s, 1H), 8.17 (d, J=1.5 Hz, 1H), 7.47 (q, J=8.3 Hz, 4H), 5.58 (s, 2H), 5.22 (s, 2H), 4.42 (q, J=6.5 Hz, 1H), 3.81 (s, 3H), 1.67 (dq, J=8.5, 4.8, 4.4 Hz, 1H), 1.39 (d, J=6.6 Hz, 6H), 0.98 (d, J=4.6 Hz, 2H), 0.76 (dd, J=8.0, 3.3 Hz, 2H). LC-MS (ESI) m/z 566.15 [M+H].

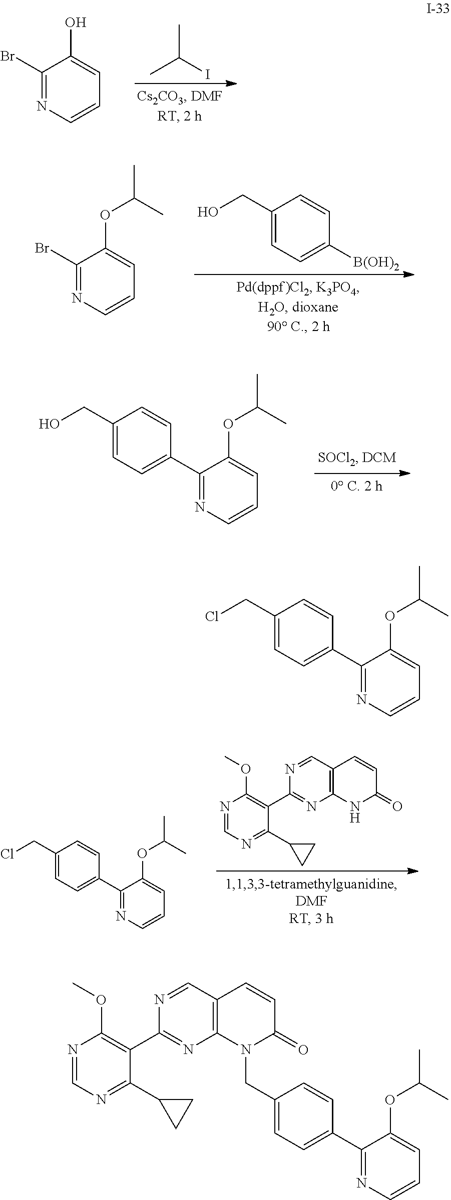

### Step 1: 2-Bromo-3-Isopropoxypyridine

To a stirred solution of 3-pyridinol, 2-bromo-(1 g, 5.747 mmol, 1 equiv) and 2-iodopropane (1.17 g, 6.896 mmol, 1.2 equiv) in DMF (10 mL) was added Cs₂CO₃ (3.75 g, 11.494 mmol, 2 equiv) at room temperature under air atmosphere. The resulting mixture was stirred for 2 h at room temperature under nitrogen atmosphere. The resulting mixture was concentrated under reduced pressure. The residue was purified by reverse flash chromatography (C18 silica gel column, eluting with 10% to 100% MeCN/water (0.1% FA) in 10 min with UV detector (254 nm)) to afford 2-bromo-3-isopropoxypyridine (540 mg, 43.48%) as a brown solid. LC-MS (ESI) m/z 216 [M+H].

### Step 2: 14-(3-Isopropoxypyridin-2-Yl)Phenyl]Methanol

To a stirred solution of 2-bromo-3-isopropoxypyridine (540 mg, 2.499 mmol, 1 equiv) and 4-(hydroxymethyl)phenylboronic acid (455.72 mg, 2.999 mmol, 1.2 equiv) in dioxane (10 mL) was added Pd(dppf)Cl₂ (182.86 mg, 0.250 mmol, 0.1 equiv), K₃PO₄ (1326.18 mg, 6.248 mmol, 2.5 equiv) and H₂O (2.5 mL) at room temperature under nitrogen atmosphere. The resulting mixture was stirred for 2 h at 90° C. under nitrogen atmosphere. The resulting mixture was concentrated under reduced pressure. The residue was purified by reverse flash chromatography (C18 silica gel column, eluting with 10% to 100% MeCN/water (0.1% FA) in 10 min with UV detector (254 nm)) to afford [4-(3-isopropoxypyridin-2-yl)phenyl]methanol (600 mg, 98.68%) as a yellow oil. LC-MS (ESI) m/z 244 [M+H].

### Step 3: 2-[4-(Chloromethyl)Phenyl]-3-Isopropoxypyridine

To a stirred solution of [4-(3-isopropoxypyridin-2-yl)phenyl]methanol (500 mg, 2.055 mmol, 1 equiv) in DCM (10 mL, 131.088 mmol, 63.79 equiv) was added thionyl chloride (2.5 mL) dropwise at 0° C. under air atmosphere. The resulting mixture was stirred for 2 h at 0° C. under nitrogen atmosphere. The residue was purified by reverse flash chromatography (C18 silica gel column, eluting with 10% to 100% MeCN/water (0.1% FA) in 10 min with UV detector (254 nm)) to afford 2-[4-(chloromethyl)phenyl]-3-isopropoxypyridine (500 mg, 92.95%) as an off-white solid. LC-MS (ESI) m/z 262 [M+H].

### Step 4: 2-(4-Cyclopropyl-6-Methoxypyrimidin-5-Yl)-8-{14-(3-Isopropoxypyridin-2-Yl)Phenyl]Methyl}Pyrido[2,3-d]Pyrimidin-7-One

To a stirred solution of 2-[4-(chloromethyl)phenyl]-4-(trifluoromethyl)pyrimidine (50 mg, 0.183 mmol, 1 equiv) and 2-(4-cyclopropyl-6-methoxypyrimidin-5-yl)-8H-pyrido[2,3-d]pyrimidin-7-one (64.98 mg, 0.220 mmol, 1.2 equiv) in DMF (1 mL) was added 1,1,3,3-tetramethylguanidine (31.68 mg, 0.274 mmol, 1.5 equiv) at room temperature under nitrogen atmosphere. The resulting mixture was stirred for 3 h at room temperature under nitrogen atmosphere. The residue was purified by reverse flash chromatography (C18 silica gel column, eluting with 10% to 100% MeCN/water (0.1% FA) in 10 min with UV detector (254 nm)) to afford 2-(4-cyclopropyl-6-methoxypyrimidin-5-yl)-8-{[4-(3-isopropoxypyridin-2-yl)phenyl]methyl pyrido[2,3-d]pyrimidin-7-one (63 mg, 63.35%) as a white solid. ¹H NMR (400 MHz, DMSO-d6) δ 9.29 (s, 1H), 8.69 (s, 1H), 8.22-8.11 (m, 2H), 7.87-7.80 (m, 2H), 7.53 (dd, J=8.6, 1.3 Hz, 1H), 7.35-7.26 (m, 3H), 6.91 (d, J=9.5 Hz, 1H), 5.55 (s, 2H), 4.70 (h, J=6.1 Hz, 1H), 3.82 (s, 3H), 1.73 (tt, J=8.2, 4.6 Hz, 1H), 1.25 (d, J=6.0 Hz, 6H), 1.00 (dq, J=6.2, 3.5 Hz, 2H), 0.75 (dq, J=7.1, 3.4 Hz, 2H). LC-MS (ESI) m/z 521.10 [M+H].

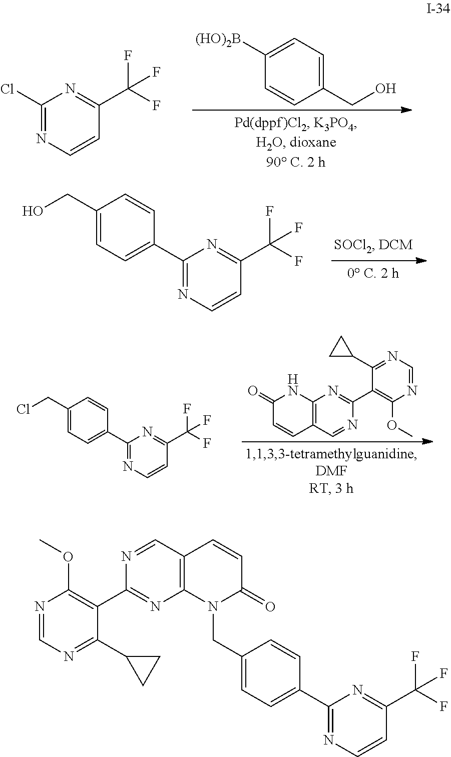

### Step 1: {4-[4-(Trifluoromethyl)Pyrimidin-2-Yl]Phenyl}Methanol

To a stirred solution of 2-chloro-4-(trifluoromethyl)pyrimidine (500 mg, 2.739 mmol, 1 equiv) and 4-(hydroxymethyl)phenylboronic acid (499.51 mg, 3.287 mmol, 1.2 equiv) in dioxane (10 mL) was added Pd(dppf)C12 (200.44 mg, 0.274 mmol, 0.1 equiv), K₃PO₄ (1453.63 mg, 6.848 mmol, 2.5 equiv) and H₂O (2 mL) at room temperature under nitrogen atmosphere. The resulting mixture was stirred for 2 h at 90° C. under nitrogen atmosphere. The resulting mixture was concentrated under reduced pressure. The residue was purified by reverse flash chromatography (C18 silica gel column, eluting with 10% to 100% MeCN/water (0.1% FA) in 10 min with UV detector (254 nm)) to afford {4-[4-(trifluoromethyl)pyrimidin-2-yl]phenyl}methanol (670 mg, 96.22%) as a brown solid. LC-MS (ESI) m/z 255 [M+H].

### Step 2: 2-[4-(Chloromethyl)Phenyl]-4-(Trifluoromethyl)Pyrimidine

To a stirred solution of {4-[4-(trifluoromethyl)pyrimidin-2-yl]phenyl}methanol (570 mg, 2.242 mmol, 1 equiv) in DCM (10 mL) was added thionyl chloride (2.5 mL, 0.004 mmol) dropwise at 0° C. under nitrogen atmosphere. The resulting mixture was stirred for 2 h at 0° C. under nitrogen atmosphere. The resulting mixture was concentrated under reduced pressure. The residue was purified by reverse flash chromatography (C18 silica gel column, eluting with 10% to 100% MeCN/water (0.1% FA) in 10 min with UV detector (254 nm)) to afford 2-[4-(chloromethyl)phenyl]-4-(trifluoromethyl)pyrimidine (440 mg, 71.97%) as a beige solid. LC-MS (ESI) m/z 273 [M+H].

### Step 3: 2-(4-Cyclopropyl-6-Methoxy-1,3-Diazinan-5-Yl)-8-({4-[4-(Trifluoromethyl)-1,3-Diazinan-2-Yl]Cyclohexyl}Methyl)-Octahydro-1H-Pyrido[2,3-d]Pyrimidin-7-Ol

To a stirred solution of 2-[4-(chloromethyl)phenyl]-4-(trifluoromethyl)pyrimidine (50 mg, 0.183 mmol, 1 equiv) and 2-(4-cyclopropyl-6-methoxypyrimidin-5-yl)-8H-pyrido[2,3-d]pyrimidin-7-one (64.98 mg, 0.220 mmol, 1.2 equiv) in DMF (1 mL) was added 1,1,3,3-tetramethylguanidine (31.68 mg, 0.274 mmol, 1.5 equiv) at room temperature under nitrogen atmosphere. The resulting mixture was stirred for 3 h at room temperature under nitrogen atmosphere. The residue was purified by reverse flash chromatography (C18 silica gel column, eluting with 10% to 100% MeCN/water (0.1% FA) in 10 min with UV detector (254 nm)) to afford 2-(4-cyclopropyl-6-m eth oxy-1,3-diazinan-5-yl)-8-({4-[4-(trifluoromethyl)-1,3-diazinan-2-yl]cyclohexyl}methyl)-octahydro-1H-pyrido[2,3-d]pyrimidin-7-ol (74 mg, 72.10%) as a white solid. ¹H NMR (300 MHz, DMSO-d6) δ 9.30 (s, 1H), 9.25 (d, J=5.0 Hz, 1H), 8.68 (s, 1H), 8.35-8.26 (m, 2H), 8.17 (d, J=9.5 Hz, 1H), 7.94 (d, J=5.0 Hz, 1H), 7.49-7.39 (m, 2H), 6.92 (d, J=9.5 Hz, 1H), 5.59 (s, 2H), 3.80 (s, 3H), 1.69 (tt, J=8.2, 4.5 Hz, 1H), 0.98 (p, J=3.4 Hz, 2H), (dq, J=6.9, 3.4 Hz, 2H). LC-MS (ESI) m/z 532.05 [M+H].

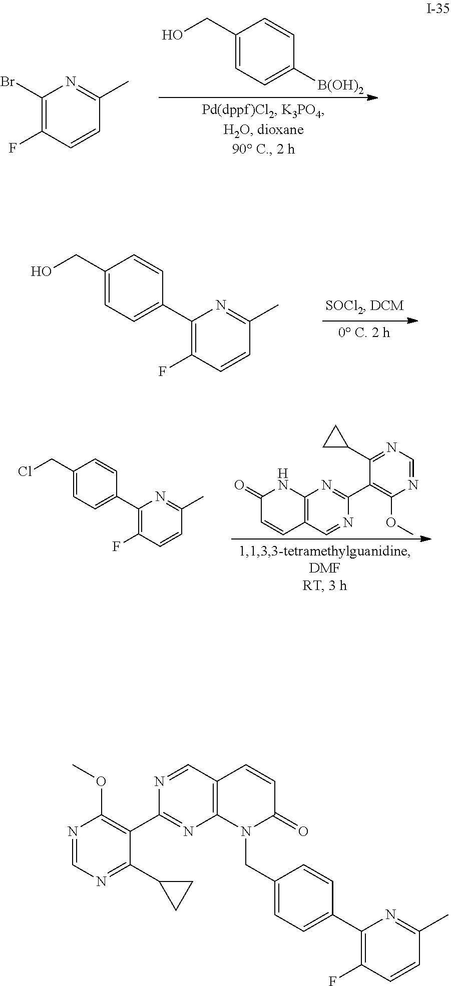

### Step 1: 14-(3-Fluoro-6-Methylpyridin-2-Yl)Phenyl]Methanol

To a stirred solution of 2-bromo-3-fluoro-6-methylpyridine (500 mg, 2.631 mmol, 1 equiv) and 4-(hydroxymethyl)phenylboronic acid (479.84 mg, 3.157 mmol, 1.2 equiv) in dioxane (10 mL) was added Pd(dppf)Cl₂ (192.54 mg, 0.263 mmol, 0.1 equiv), K₃PO₄ (1396.37 mg, 6.577 mmol, 2.5 equiv) and H₂O (2 mL) at room temperature under nitrogen atmosphere. The resulting mixture was stirred for 2 h at 90° C. under nitrogen atmosphere. The resulting mixture was concentrated under reduced pressure. The residue was purified by reverse flash chromatography (C18 silica gel column, eluting with 10% to 100% MeCN/water (0.1% FA) in 10 min with UV detector (254 nm)) to afford [4-(3-fluoro-6-methylpyridin-2-yl)phenyl]methanol (550 mg, 96.21%) as a brown solid. LC-MS (ESI) m/z 218 [M+H].

### Step 2: 2-[4-(Chloromethyl)Phenyl]-3-Fluoro-6-Methylpyridine

To a stirred solution of [4-(3-fluoro-6-methylpyridin-2-yl)phenyl]methanol (450 mg, 2.071 mmol, 1 equiv) in DCM (9 mL) was added thionyl chloride (2.5 mL, 0.004 mmol) dropwise at 0° C. under nitrogen atmosphere. The resulting mixture was stirred for 2 h at 0° C. under nitrogen atmosphere. The residue was purified by reverse flash chromatography (C18 silica gel column, eluting with 10% to 100% MeCN/water (0.1% FA) in 10 min with UV detector (254 nm)) to afford 2-[4-(chloromethyl)phenyl]-3-fluoro-6-methylpyridine (480 mg, 98.32%) as a beige solid. LC-MS (ESI) m/z 236 [M+H].

### Step 3: 2-(4-Cyclopropyl-6-Methoxypyrimidin-5-Yl)-8-{14-(3-Fluoro-6-Methylpyridin-2-Yl)Phenyl]Methyl}Pyrido[2,3-d]Pyrimidin-7-One

To a stirred solution of 2-[4-(chloromethyl)phenyl]-3-fluoro-6-methylpyridine (50 mg, 0.212 mmol, 1 equiv) and 2-(4-cyclopropyl-6-methoxypyrimidin-5-yl)-8H-pyrido[2,3-d]pyrimidin-7-one (75.18 mg, 0.254 mmol, 1.2 equiv) in DMF (1 mL) was added 1,1,3,3-tetramethylguanidine (36.65 mg, 0.318 mmol, 1.5 equiv) at room temperature under nitrogen atmosphere. The resulting mixture was stirred for 3 h at room temperature under nitrogen atmosphere. The residue was purified by reverse flash chromatography (C18 silica gel column, eluting with 10% to 100% MeCN/water (0.1% FA) in 10 min with UV detector (254 nm)) to afford 2-(4-cyclopropyl-6-methoxypyrimidin-5-yl)-8-{[4-(3-fluoro-6-methylpyridin-2-yl)phenyl]methyl pyrido[2,3-d]pyrimidin-7-one (69 mg, 65.77%) as a white solid. ¹H NMR (300 MHz, Chloroform-d) δ 9.01 (s, 1H), 8.72 (s, 1H), 7.85 (d, J=7.8 Hz, 2H), 7.77 (d, J=9.5 Hz, 1H), 7.62 (d, J=7.9 Hz, 2H), 7.37 (dd, J=10.7, 8.3 Hz, 1H), 7.10 (dd, J=8.4, 3.3 Hz, 1H), 6.89 (d, J=9.5 Hz, 1H), 5.73 (s, 2H), 3.95 (s, 3H), 2.60 (s, 3H), 1.69 (dq, J=8.2, 4.1 Hz, 1H), 1.26 (dd, J=4.5, 2.8 Hz, 2H), 0.89 (dq, J=7.0, 3.9 Hz, 2H). LC-MS (ESI) m/z 495.10 [M+H].

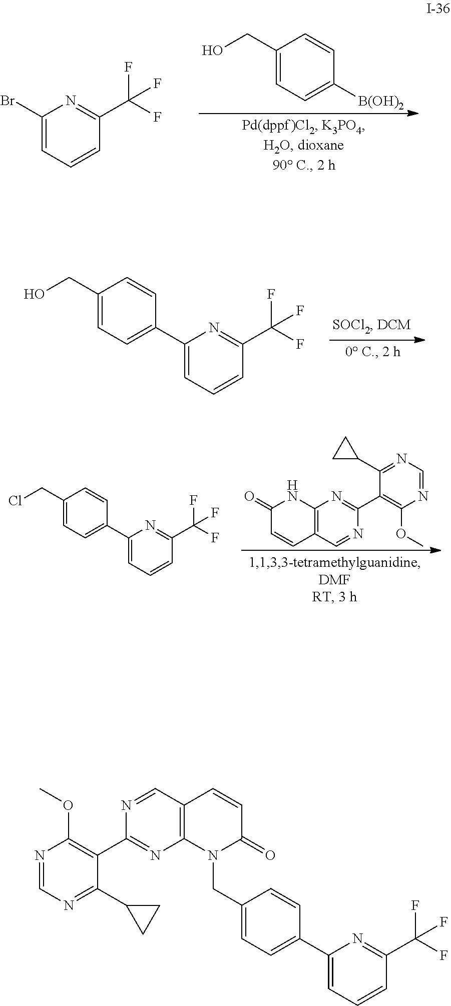

### Step 1: {4-[6-(Trifluoromethyl)Pyridin-2-Yl]Phenyl}Methanol

To a stirred solution of 2-bromo-6-(trifluoromethyl)pyridine (500 mg, 2.212 mmol, 1 equiv) and 4-(hydroxymethyl)phenylboronic acid (403.44 mg, 2.654 mmol, 1.2 equiv) in dioxane (10 mL) was added Pd(dppf)Cl₂ (161.89 mg, 0.221 mmol, 0.1 equiv), K₃PO₄ (1174.05 mg, 5.530 mmol, 2.5 equiv) and H₂O (2 mL) at room temperature under nitrogen atmosphere. The resulting mixture was stirred for 2 h at 90° C. under nitrogen atmosphere. The resulting mixture was concentrated under reduced pressure. The residue was purified by reverse flash chromatography (C18 silica gel column, eluting with 10% to 100% MeCN/water (0.1% FA) in 30 min with UV detector (254 nm)) to afford {4-[6-(trifluoromethyl)pyridin-2-yl]phenyl}methanol (550 mg, 98.17%) as a brown solid. LC-MS (ESI) m/z 254 [M+H].

### Step 2: 2-[4-(Chloromethyl)Phenyl]-6-(Trifluoromethyl)Pyridine

To a stirred solution of {4-[6-(trifluoromethyl)pyridin-2-yl]phenyl}methanol (450 mg, 1.777 mmol, 1 equiv) in DCM (9 mL) was added thionyl chloride (2.5 mL) dropwise at 0° C. under nitrogen atmosphere. The resulting mixture was stirred for 2 h at 0° C. under air atmosphere. The residue was purified by reverse flash chromatography (C18 silica gel column, eluting with 10% to 100% MeCN/water (0.1% FA) in 10 min with UV detector (254 nm)) to afford 2-[4-(chloromethyl)phenyl]-6-(trifluoromethyl)pyridine (450 mg, 93.21%) as a beige solid. LC-MS (ESI) m/z 272 [M+H].

### Step 3: 2-(4-Cyclopropyl-6-Methoxypyrimidin-5-Yl)-8-({4-[6-(Trifluoromethyl)Pyridin-2-Yl]Phenyl}Methyl)Pyrido[2,3-d]Pyrimidin-7-One

To a stirred solution of 2-[4-(chloromethyl)phenyl]-6-(trifluoromethyl)pyridine (50 mg, 0.184 mmol, 1 equiv) and 2-(4-cyclopropyl-6-methoxypyrimidin-5-yl)-8H-pyrido[2,3-d]pyrimidin-7-one (65.22 mg, 0.221 mmol, 1.2 equiv) in DMF (1 mL) was added 1,1,3,3-tetramethylguanidine (31.80 mg, 0.276 mmol, 1.5 equiv) at room temperature under nitrogen atmosphere. The resulting mixture was stirred for 3 h at room temperature under nitrogen atmosphere. The residue was purified by reverse flash chromatography (C18 silica gel column, eluting with 10% to 100% MeCN/water (0.1% FA) in 10 min with UV detector (254 nm)) to afford 2-(4-cyclopropyl-6-methoxypyrimidin-5-yl)-8-({4-[6-(trifluoromethyl)pyridin-2-yl]phenyl}methyl)pyrido[2,3-d]pyrimidin-7-one (66 mg, 67.60%) as a white solid. ¹H NMR (300 MHz, Chloroform-d) δ 9.02 (s, 1H), 8.74 (s, 1H), 7.99-7.85 (m, 4H), 7.79 (d, J=9.5 Hz, 1H), 7.61 (dd, J=7.6, 3.4 Hz, 3H), 6.91 (d, J=9.5 Hz, 1H), 5.74 (s, 2H), 3.96 (s, 3H), 1.70 (s, 1H), 1.30 (s, 2H), 0.89 (d, J=7.7 Hz, 2H). LC-MS (ESI) m/z 531.05 [M+H].

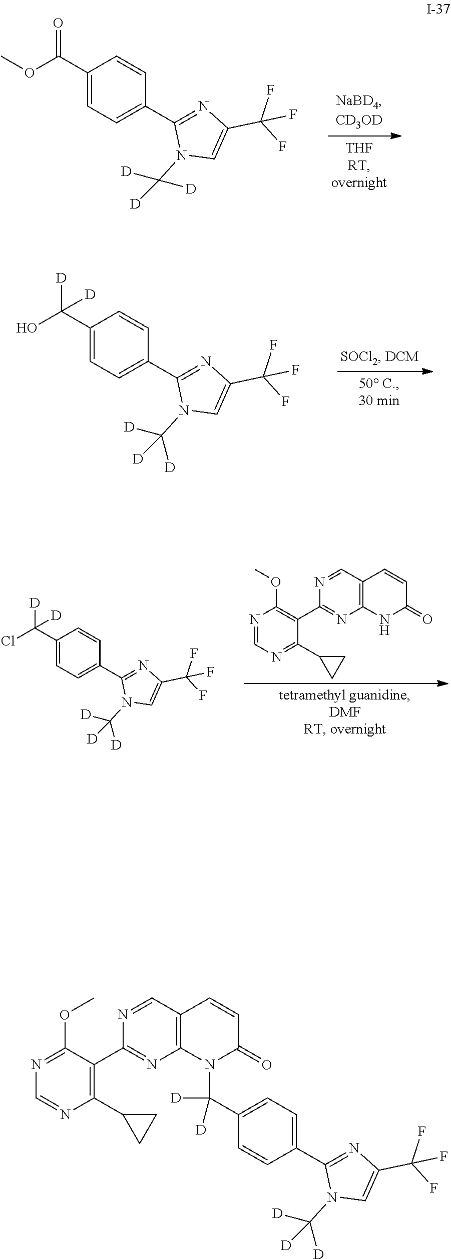 [SMILES: [2H]C([2H])([2H])n1cc(C(F)(F)F)nc1-c1ccc(C(=O)OC)cc1 ; [2H]C([2H])(O)c1ccc(-c2nc(C(F)(F)F)cn2C([2H])([2H])[2H])cc1 ; [2H]C([2H])(Cl)c1ccc(-c2nc(C(F)(F)F)cn2C([2H])([2H])[2H])cc1 ; COc1ncnc(C2CC2)c1-c1ncc2ccc(=O)[nH]c2n1 ; [2H]C([2H])([2H])n1cc(C(F)(F)F)nc1-c1ccc(C([2H])([2H])n2c(=O)ccc3cnc(-c4c(OC)ncnc4C4CC4)nc32)cc1]

### Step 1: {4-[1-(2H3)Methyl-4-(Trifluoromethyl)Imidazol-2-Yl]Phenyl}(2H2)Methanol

To a stirred solution of methyl 4-[1-(2H3)methyl-4-(trifluoromethyl)imidazol-2-yl]benzoate (300 mg, 1.044 mmol, 1 equiv) in THF (6 mL, 74.057 mmol, 70.91 equiv) and CD 3 OD (0.6 mL, 13.475 mmol, 12.90 equiv) was added sodium (2H4)boranuide (174.86 mg, 4.176 mmol, 4 equiv) in portions at 0° C. under nitrogen atmosphere. The resulting mixture was stirred for overnight at room temperature under nitrogen atmosphere. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford {4-[1-(2H3)methyl-4-(trifluoromethyl)imidazol-2-yl]phenyl}(2H2)methanol (257 mg, 94.19%) as a yellow solid. LC-MS (ESI) m/z 262.1 [M+H]

### Step 2: 2-{4-[Chloro(2H2)Methyl]Phenyl]-1-(2H3)Methyl-4-(Trifluoromethyl)Imidazole

To a stirred mixture of {4-[1-(2H3)methyl-4-(trifluoromethyl)imidazol-2-yl]phenyl}(2H2)methanol (50 mg, 0.191 mmol, 1 equiv) in DCE (1 mL, 12.633 mmol, 66.01 equiv) was added SOCl₂ (68.30 mg, 0.573 mmol, 3 equiv) dropwise at room temperature under nitrogen atmosphere. The resulting mixture was stirred for 30 min at under nitrogen atmosphere. The resulting mixture was extracted with EtOAc (3×5 mL). The combined organic layers were washed with brine (3×5 mL), dried over anhydrous Na₂SO₄, filtered and concentrated under reduced pressure to afford 2-{4-[chloro(2H2)methyl]phenyl}-1-(2H3)methyl-4-(trifluoromethyl)imidazole (50 mg, 93.41%) as a yellow solid. LC-MS (ESI) m/z 280.1 [M+H]

### Step 3: 2-(4-Cyclopropyl-6-Methoxypyrimidin-5-Yl)-8-({4-[1-(2H3)Methyl-4-(Trifluoromethyl)Imidazol-2-Yl]Phenyl}(2H2)Methyl)Pyrido[2,3-d]Pyrimidin-7-One

To a stirred mixture of 2-{4-[chloro(2H2)methyl]phenyl}-1-(2H3)methyl-4-(trifluoromethyl)imidazole (34.65 mg, 0.124 mmol, 0.9 equiv) and 2-(4-cyclopropyl-6-methoxypyrimidin-5-yl)-8H-pyrido[2,3-d]pyrimidin-7-one (40.65 mg, 0.138 mmol, 1.00 equiv) in DMF (0.8 mL, 10.337 mmol, 75.09 equiv) was added 1,1,3,3-tetramethylguanidine (23.78 mg, 0.207 mmol, 1.5 equiv) dropwise at room temperature under nitrogen atmosphere. The resulting mixture was stirred for overnight at room temperature under nitrogen atmosphere. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[1-(2H3)methyl-4-(trifluoromethyl)imidazol-2-yl]phenyl (2H2)methyl)pyrido[2,3-d]pyrimidin-7-one (57.9 mg, 77.48%) as an off-white solid.

¹H NMR (400 MHz, DMSO-d6) δ 9.29 (s, 1H), 8.69 (s, 1H), 8.16 (d, J=9.5 Hz, 1H), 7.91 (d, J=1.5 Hz, 1H), 7.66-7.60 (m, 2H), 7.43-7.38 (m, 2H), 6.90 (d, J=9.5 Hz, 1H), 3.83 (s, 3H), 1.71 (dt, J=8.1, 4.5 Hz, 1H), 1.05-0.98 (m, 2H), 0.80-0.72 (m, 2H). LC-MS (ESI) m/z 539.2 [M+H].

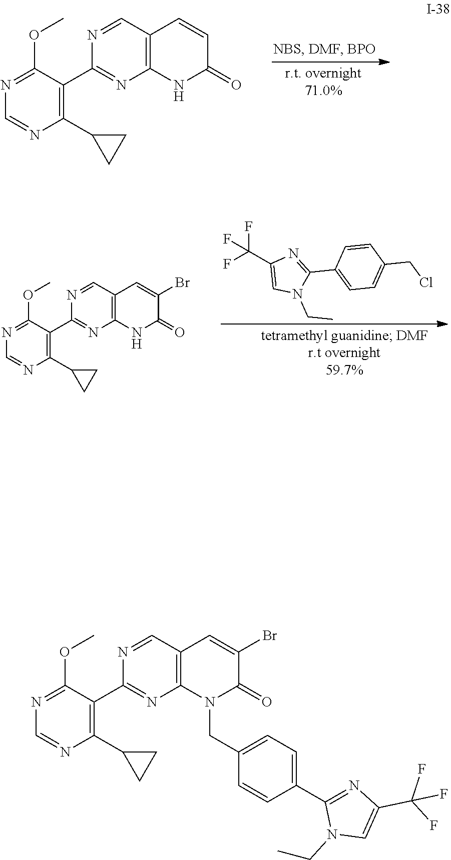 [SMILES: COc1ncnc(C2CC2)c1-c1ncc2ccc(=O)[nH]c2n1 ; COc1ncnc(C2CC2)c1-c1ncc2cc(Br)c(=O)[nH]c2n1 ; CCn1cc(C(F)(F)F)nc1-c1ccc(CCl)cc1 ; CCn1cc(C(F)(F)F)nc1-c1ccc(Cn2c(=O)c(Br)cc3cnc(-c4c(OC)ncnc4C4CC4)nc32)cc1]

### Step 1: 6-Bromo-2-(4-Cyclopropyl-6-Methoxypyrimidin-5-Yl)-811-Pyrido[2,3-d]Pyrimidin-7-One

To a stirred solution of 2-(4-cyclopropyl-6-methoxypyrimidin-5-yl)-8H-pyrido[2,3-d]pyrimidin-7-one (1 g, 3.386 mmol, 1 equiv) and NBS (1.81 g, 10.158 mmol, 3 equiv) in DMF (15 mL) was added BPO (0.17 g, 0.677 mmol, 0.2 equiv) at room temperature. The resulting mixture was stirred for overnight at room temperature under nitrogen atmosphere and concentrated under reduced pressure. The residue was purified by reverse flash chromatography (C18 silica gel column, eluting with 0% to 100% MeCN/water (0.1% TFA) in 10 min with UV detector (254 nm)) to afford 6-bromo-2-(4-cyclopropyl-6-methoxypyrimidin-5-yl)-8H-pyrido[2,3-d]pyrimidin-7-one (900 mg, 71.0%) as a yellow solid. LC-MS (ESI) m/z 374.2 [M+H].

### Step2: 6-Bromo-2-(4-Cyclopropyl-6-Methoxypyrimidin-5-Yl)-8-({4-[1-Ethyl-4-(Trifluoromethyl)Imidazol-2-Yl]Phenyl}Methyl)Pyrido[2,3-d]Pyrimidin-7-One

To a stirred solution of 6-bromo-2-(4-cyclopropyl-6-methoxypyrimidin-5-yl)-8H-pyrido[2,3-d]pyrimidin-7-one (150 mg, 0.401 mmol, 1 equiv) and 2-[4-(chloromethyl)phenyl]-1-ethyl-4-(trifluoromethyl)imidazole (104.15 mg, 0.361 mmol, 0.9 equiv) in DMF (2 mL) was added 1,1,3,3-tetramethylguanidine (69.25 mg, 0.602 mmol, 1.5 equiv) dropwise at room temperature. The resulting mixture was stirred for overnight at room temperature under nitrogen atmosphere. The residue was purified by reverse flash chromatography (C18 silica gel column, eluting with 0% to 100% MeCN/water (0.1% FA) in 10 min with UV detector (254 nm)) to afford 6-bromo-2-(4-cyclopropyl-6-methoxypyrimidin-5-yl)-8-({4-[1-ethyl-4-(trifluoromethyl)imidazo]-2-yl]phenyl}methyl)pyrido[2,3-d]pyrimidin-7-one (150 mg, 59.7%) as a yellow solid. ¹-HNMR (300 MHz, DMSO-d6) δ 9.29 (s, 1H), 8.78 (s, 1H), 8.70 (s, 1H), 8.02 (d, J=1.4 Hz, 1H), 7.56 (d, J=8.2 Hz, 2H), 7.43 (d, J=8.1 Hz, 2H), 5.64 (s, 2H), 4.05 (q, J=7.2 Hz, 2H), 3.84 (s, 3H), 1.85-1.66 (m, 1H), 1.30 (t, J=7.2 Hz, 3H), 1.02 (m, 2H), 0.77 (m, 2H). LC-MS (ESI) m/z 627.85 [M+H].

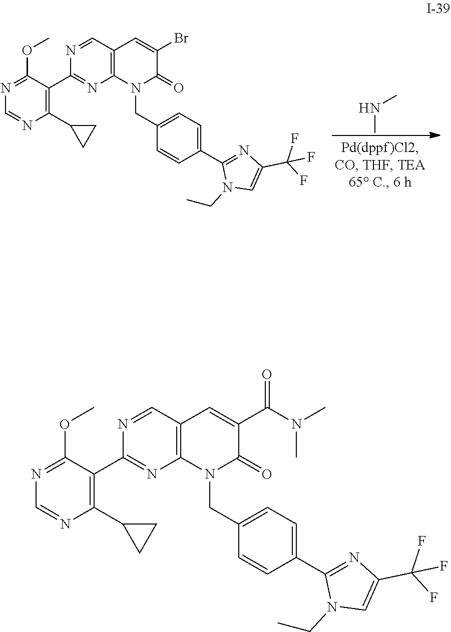

To a stirred solution of 6-bromo-2-(4-cyclopropyl-6-methoxypyrimidin-5-yl)-8-({4-[1-ethyl-4-(trifluoromethyl)imidazo]-2-yl]phenyl}methyl)pyrido[2,3-d]pyrimidin-7-one (80 mg, 0.128 mmol, 1 equiv) and Me 2 NH (2 mL, 2 M in THF) was added TEA (64.61 mg, 0.640 mmol, 5 equiv) and Pd(dppf)C12 (11.68 mg, 0.016 mmol, 0.2 equiv) at room temperature. The resulting mixture was stirred for 6 h at 65° C. under carbon monoxide atmosphere. The resulting mixture was filtered, the filter cake was washed with 1,4-dioxane (3×20 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[1-ethyl-4-(trifluoromethyl)imidazo]-2-yl]phenyl methyl)-N,N-dimethyl-7-oxopyrido[2,3-d]pyrimidine-6-carb oxamide (19.3 mg, 23.9%) as an off-white solid. ¹H NMR (400 MHz, DMSO-d6) δ 9.31 (s, 1H), 8.70 (s, 1H), 8.22 (s, 1H), 8.04-7.99 (s, 1H), 7.59-7.52 (s, 2H), 7.43 (d, J=8.1 Hz, 2H), (s, 2H), 4.04 (q, J=7.2 Hz, 2H), 3.84 (s, 3H), 2.99 (s, 3H), 2.90 (s, 3H), 1.74 (m, 1H), 1.29 (t, J=7.3 Hz, 3H), 1.02 (p, J=3.5 Hz, 2H), 0.77 (m, 2H). LC-MS (ESI) m/z: 619.0 [M+H].

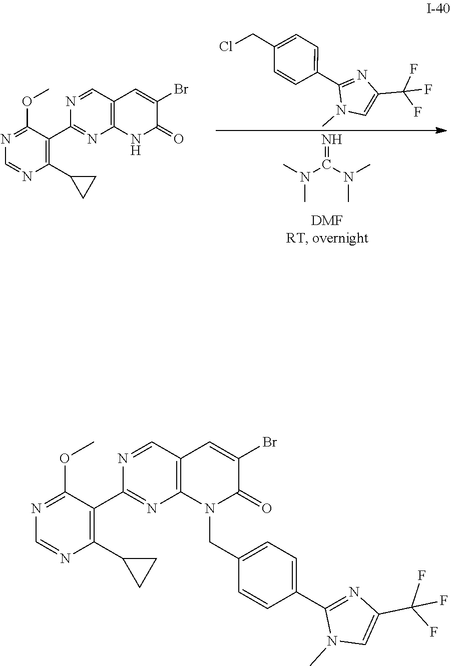

To a stirred solution of 6-bromo-2-(4-cyclopropyl-6-methoxypyrimidin-5-yl)-8H-pyrido[2,3-d]pyrimidin-7-one (50 mg, 0.134 mmol, 1 equiv) and 2-[4-(chloromethyl)phenyl]-1-methyl-4-(trifluoromethyl)imidazole (33.03 mg, 0.121 mmol, 0.9 equiv) in DMF (1 mL) was added 1,1,3,3-tetramethylguanidine (23.09 mg, 0.201 mmol, 1.5 equiv) at room temperature under nitrogen atmosphere. The resulting mixture was stirred for overnight at room temperature under nitrogen atmosphere. The mixture was purified by reverse flash chromatography (C18 silica gel column, eluting with 0% to 100% MeCN/water (0.1% FA) in 10 min with UV detector (254 nm)) to afford 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (13.7 mg, 16.61%) as an off-white solid. ¹H NMR: (400 MHz, DMSO-d6) δ 9.29 (s, 1H), 8.73 (d, J=29.9 Hz, 2H), 7.92 (s, 1H), 7.64 (d, J=7.9 Hz, 2H), 7.43 (d, J=7.8 Hz, 2H), 5.63 (s, 2H), 3.84 (s, 3H), 3.74 (s, 3H), 1.74 (td, J=8.4, 4.4 Hz, 1H), 1.07-0.97 (m, 2H), 0.77 (dd, J=7.9, 3.5 Hz, 2H). LC-MS (ESI) m/z 612.4 [M+H].

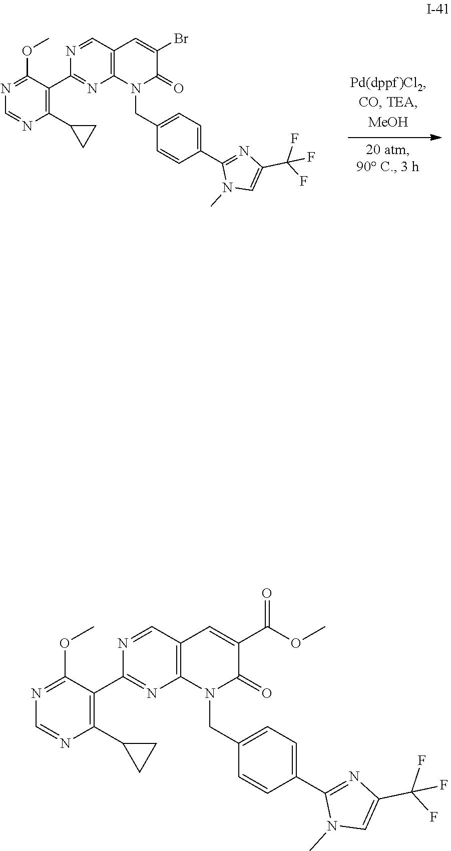 [SMILES: COc1ncnc(C2CC2)c1-c1ncc2cc(Br)c(=O)n(Cc3ccc(-c4nc(C(F)(F)F)cn4C)cc3)c2n1 ; COC(=O)c1cc2cnc(-c3c(OC)ncnc3C3CC3)nc2n(Cc2ccc(-c3nc(C(F)(F)F)cn3C)cc2)c1=O]

To a solution of 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (50 mg, mmol, 1 equiv) in 3 mL MeOH was added TEA (41.31 mg, 0.410 mmol, 5 equiv) and Pd(dppf)Cl₂ (11.95 mg, 0.016 mmol, 0.2 equiv) in a pressure tank. The mixture was purged with nitrogen for 1 min and then was pressurized to 20 atm with carbon monoxide at 90° C. for 3 h. The reaction mixture was cooled to room temperature and filtered to remove insoluble solids. The filtrate was concentrated under reduced pressure. The residue was purified by reverse flash chromatography (C18 silica gel column, eluting with 0% to 100% MeCN/water (0.1% FA) in 10 min with UV detector (254 nm)) to afford to afford methyl 2-(4-cyclopropyl-6-methoxypyrimidin-d]pyrimidine-6-carboxylate (14.2 mg, 28.55%) as an off-white solid. ¹H NMR (400 MHz, DMSO-d6) δ 9.43 (s, 1H), 8.74 (d, J=28.8 Hz, 2H), 7.95-7.85 (m, 1H), 7.64 (d, J=8.2 Hz, 2H), 7.43 (d, J=8.0 Hz, 2H), 5.58 (s, 2H), 3.85 (d, J=7.5 Hz, 6H), 3.74 (s, 3H), 1.77 (tt, J=8.3, 4.7 Hz, 1H), 1.03 (p, J=3.5 Hz, 2H), 0.78 (dq, J=6.8, 3.4 Hz, 2H). LC-MS (ESI) m/z 591.5 [M+H]

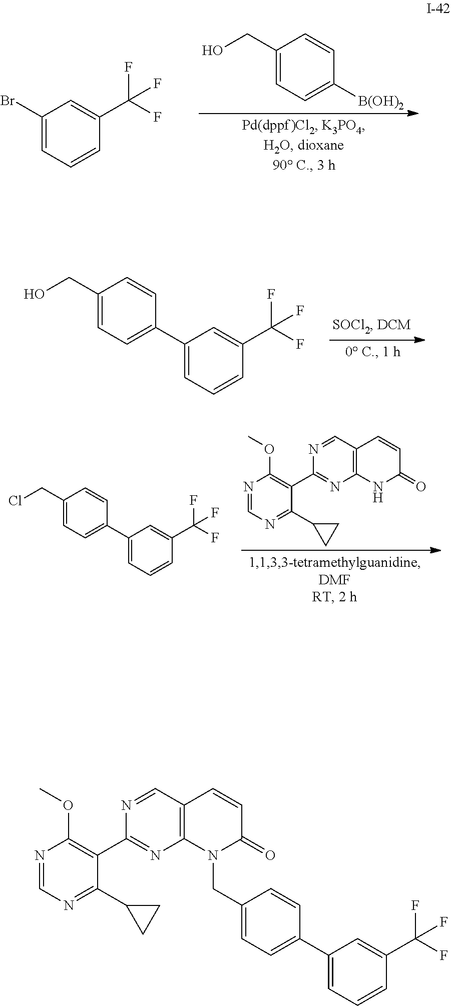

### Step 1: 13′-(Trifluoromethyl)-11,1′-Biphenyl]-4-Yl]Methanol

To a stirred solution of 1-bromo-3-(trifluoromethyl)benzene (300 mg, 1.333 mmol, 1 equiv) and 4-(hydroxymethyl)phenylboronic acid (243.13 mg, 1.600 mmol, 1.2 equiv) in dioxane (5 mL) was added Pd(dppf)Cl₂ (97.56 mg, 0.133 mmol, 0.1 equiv), K₃PO₄ (707.52 mg, 3.333 mmol, 2.5 equiv) and H₂O (1.5 mL) at room temperature under air atmosphere. The resulting mixture was stirred for 3 h at 90° C. under nitrogen atmosphere. The residue was purified by reverse flash chromatography (C18 silica gel column, eluting with 10% to 100% MeCN/water (0.1% FA) in 10 min with UV detector (254 nm)) to afford [3′-(trifluoromethyl)-[1,1′-biphenyl]-4-yl]methanol (300 mg, 89.21%) as a Brown yellow solid. LC-MS (ESI) m/z 253 [M+H].

### Step 2: 4′-(Chloromethyl)-3-(Trifluoromethyl)-1,1′-Biphenyl

To a stirred solution of [3′-(trifluoromethyl)-[1,1′-biphenyl]-4-yl]methanol (300 mg, 1.189 mmol, 1 equiv) in DCM (5 mL) was added thionyl chloride (2 mL, 0.017 mmol) dropwise at 0° C. under air atmosphere. The resulting mixture was stirred for 1 h at 0° C. under nitrogen atmosphere. The resulting mixture was concentrated under reduced pressure. The residue was purified by reverse flash chromatography (C18 silica gel column, eluting with 10% to 100% MeCN/water (0.1% FA) in 10 min with UV detector (254 nm)) to afford 4′-(chloromethyl)-3-(trifluoromethyl)-1,1′-biphenyl (280 mg, 86.97%) as a brown yellow solid. LC-MS (ESI) m/z 271 [M+H].

### Step 3: 2-(4-Cyclopropyl-6-Methoxypyrimidin-5-Yl)-8-{13′-(Trifluoromethyl)-11,1′-Biphenyl]-4-Yl]Methyl}Pyrido[2,3-d]Pyrimidin-7-One

To a stirred solution of 4′-(chloromethyl)-3-(trifluoromethyl)-1,1′-biphenyl (55 mg, mmol, 1 equiv) and 2-(4-cyclopropyl-6-methoxypyrimidin-5-yl)-8H-pyrido[2,3-d]pyrimidin-7-one (54.00 mg, 0.183 mmol, 0.9 equiv) in DMF (1 mL) was added 1,1,3,3-tetramethylguanidine (35.10 mg, 0.304 mmol, 1.5 equiv) at room temperature. The resulting mixture was stirred for 2 h at room temperature under nitrogen atmosphere. The resulting mixture was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 63% B to 77% B in 7 min, 77% B; Wave Length: 220 nm; RT1(min): 6.32;) to afford 2-(4-cyclopropyl-6-methoxypyrimidin-5-yl)-8-{[3′-(trifluoromethyl)-[1,1′-biphenyl]-4-yl]methyl pyrido[2,3-d]pyrimidin-7-one (55 mg, 51.12%) as a white solid. ¹H NMR (400 MHz, DMSO-d6) δ 9.29 (s, 1H), 8.69 (s, 1H), 8.16 (d, J=9.5 Hz, 1H), 7.99-7.87 (m, 2H), 7.74-7.61 (m, 4H), 7.39 (d, J=8.2 Hz, 2H), 6.90 (d, J=9.5 Hz, 1H), 5.56 (s, 2H), 3.83 (s, 3H), 1.71 (tt, J=8.1, 4.5 Hz, 1H), 1.01 (dq, J=6.1, 3.5 Hz, 2H), 0.75 (dq, J=6.9, 3.4 Hz, 2H). LC-MS (ESI) m/z 530.10 [M+H].

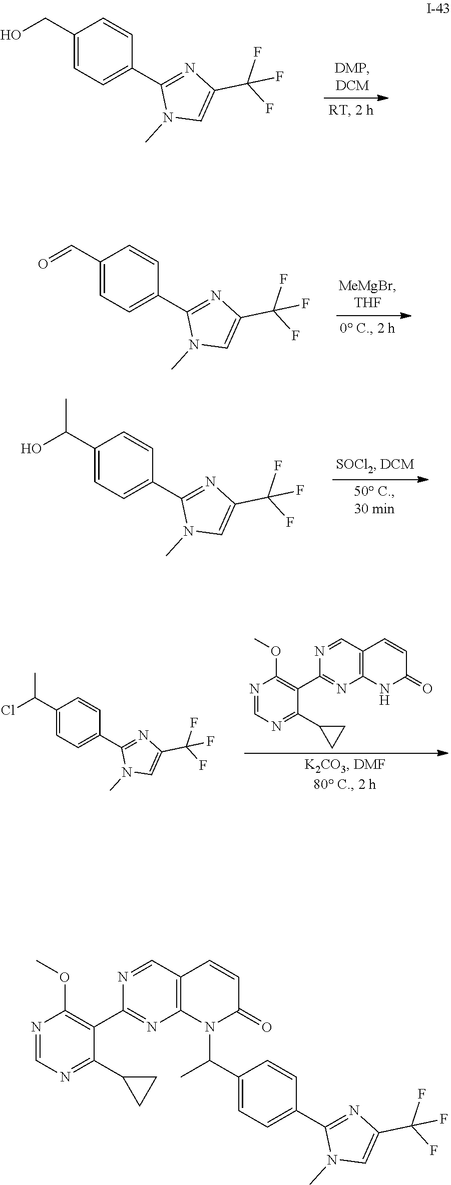 [SMILES: Cn1cc(C(F)(F)F)nc1-c1ccc(CO)cc1 ; Cn1cc(C(F)(F)F)nc1-c1ccc(C=O)cc1 ; CC(O)c1ccc(-c2nc(C(F)(F)F)cn2C)cc1 ; CC(Cl)c1ccc(-c2nc(C(F)(F)F)cn2C)cc1 ; COc1ncnc(C2CC2)c1-c1ncc2ccc(=O)[nH]c2n1 ; COc1ncnc(C2CC2)c1-c1ncc2ccc(=O)n(C(C)c3ccc(-c4nc(C(F)(F)F)cn4C)cc3)c2n1]

### Step 1: 4-[1-Methyl-4-(Trifluoromethyl)Imidazol-2-Yl]Benzaldehyde

To a stirred mixture of {4-[1-methyl-4-(trifluoromethyl)imidazol-2-yl]phenyl}methanol (1.8 g, 7.025 mmol, 1 equiv) in DCM (30.34 mL, 477.208 mmol, 67.93 equiv) was added DMP (5.96 g, 14.050 mmol, 2 equiv) in portions at room temperature under nitrogen atmosphere. The resulting mixture was stirred for 2 h at room temperature under nitrogen atmosphere. The resulting mixture was extracted with EtOAc (3×100 mL). The combined organic layers were washed with brine (3×100 mL), dried over anhydrous Na₂SO₄, filtered and concentrated under reduced pressure. The residue was purified by silica gel column chromatography, (eluting with PE/EA (4:1)) to afford 4-[1-methyl-4-(trifluoromethyl)imidazol-2-yl]benzaldehyde (1.76 g, 98.55%) as a yellow oil. LC-MS (ESI) m/z 255.1 [M+H]

### Step 2: 1-{4-[1-Methyl-4-(Trifluoromethyl)Imidazol-2-Yl]Phenyl}Ethanol

To a stirred mixture of 4-[1-methyl-4-(trifluoromethyl)imidazol-2-yl]benzaldehyde (1.76 g, 6.923 mmol, 1 equiv) in THF (35 mL, 431.997 mmol, 62.40 equiv) was added bromo(methyl)magnesium (6.92 mL, 20.769 mmol, 3 equiv) dropwise at under nitrogen atmosphere. The resulting mixture was stirred for 2h at under nitrogen atmosphere. The resulting mixture was extracted with EtOAc (3×100 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 1-{4-[1-methyl-4-(trifluoromethyl)imidazo]-2-yl]phenyl}ethanol (1.6 g, 85.51%) as a yellow oil. LC-MS (ESI) m/z 271.1 [M+H]

### Step 3: 2-[4-(1-Chloroethyl)Phenyl]-1-Methyl-4-(Trifluoromethyl)Imidazole

To a stirred mixture of 1-{4-[1-methyl-4-(trifluoromethyl)imidazol-2-yl]phenyl}ethanol (600 mg, 2.220 mmol, 1 equiv) in DCE (12 mL, 151.592 mmol, 68.28 equiv) was added thionyl chloride (792.32 mg, 6.660 mmol, 3 equiv) dropwise at room temperature under nitrogen atmosphere. The resulting mixture was stirred for 30 min at under nitrogen atmosphere. The resulting mixture was extracted with EtOAc (3×50 mL). The combined organic layers were washed with brine (3×10 mL), dried over anhydrous Na₂SO₄, filtered and concentrated under reduced pressure to afford 2-[4-(1-chloroethyl)phenyl]-1-methyl-4-(trifluoromethyl)imidazole (640 mg, 99.85%) as a yellow oil. LC-MS (ESI) m/z 289.1 [M+H]

### Step 4: 2-(4-Cyclopropyl-6-Methoxypyrimidin-5-Yl)-8-(1-{4-[1-Methyl-4-(Trifluoromethyl)Imidazol-2-Yl]Phenyl}Ethyl)Pyrido[2,3-d]Pyrimidin-7-One

A mixture of 2-[4-(1-chloroethyl)phenyl]-1-methyl-4-(trifluoromethyl)imidazole (117.32 mg, 0.407 mmol, 1.2 equiv),2-(4-cyclopropyl-6-methoxypyrimidin-5-yl)-8H-pyrido[2,3-d]pyrimidin-7-one (100 mg, 0.339 mmol, 1.00 equiv) and K₂CO₃ (140.40 mg, 1.017 mmol, 3 equiv) in DMF (2 mL, 25.843 mmol, 76.32 equiv) was stirred for 2h at under nitrogen atmosphere. The resulting mixture was extracted with EtOAc (3×10 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-(1-{4-[1-methyl-4-(trifluoromethyl)imidazol-2-yl]phenyl ethyl)pyrido[2,3-d]pyrimidin-7-one (50 mg, 26.64%) as an off-white solid. ¹H NMR (400 MHz, DMSO-d6) δ 9.27 (s, 1H), 8.69 (s, 1H), 8.12 (d, J=9.5 Hz, 1H), 7.92 (s, 1H), 7.61 (d, J=8.0 Hz, 2H), 7.38 (d, J=8.0 Hz, 2H), 6.82 (s, 2H), 3.92 (s, 3H), 3.74 (s, 3H), 1.95 (d, J=7.0 Hz, 3H), 1.77 (s, 1H), 1.02 (s, 2H), 0.83 (d, J=9.5 Hz, 2H). LC-MS (ESI) m/z 548.2 [M+H].

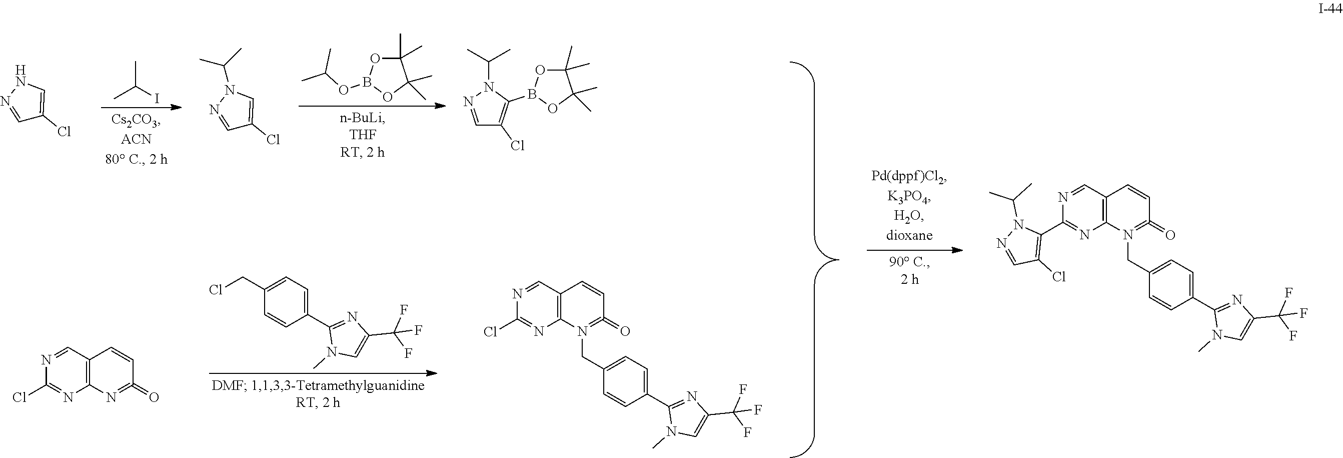

### Step 1: 4-Chloro-1-Isopropylpyrazole

A solution of 1H-pyrazole, 4-chloro-(9CI) (1 g, 9.754 mmol, 1 equiv), Cs₂CO₃ (6.36 g, 19.508 mmol, 2.0 equiv) in ACN (15 mL, 285.363 mmol, 29.26 equiv) was treated with 2-iodopropane (1.46 mL, 14.601 mmol, 1.50 equiv) for 2 h at 80° C. under nitrogen atmosphere. The mixture was allowed to cool down to room temperature. The resulting mixture was filtered, the filter cake was washed with EtOAc (3×20 mL), filtered and concentrated under reduced pressure. The residue was purified by silica gel column chromatography, (eluting with PE/EA (10:1)) to afford 4-chloro-1-isopropylpyrazole (0.79 g, 56.01%) as a green liquid. LCMS (ESI) m/z 144.05 [M+H].

### Step 2: 4-Chloro-1-Isopropyl-5-(4,4,5,5-Tetramethyl-1,3,2-Dioxaborolan-2-Yl)Pyrazole

To a stirred solution of 1-isopropyl-4-methylpyrazole (1.2 g, 9.663 mmol, 1 equiv) in THF (15 mL, 185.142 mmol, 33.89 equiv) were added n-BuLi (2.62 mL, 6.556 mmol, 1.2 equiv) dropwise at 0° C. under nitrogen atmosphere. The resulting mixture was stirred for 1 h at room temperature under nitrogen atmosphere. To the above mixture was added 2-isopropoxy-4,4,5,5-tetramethyl-1,3,2-dioxaborolane (1.22 g, 6.556 mmol, 1.2 equiv) dropwise over at −78° C. The resulting mixture was stirred for additional 2h at room temperature. The reaction was quenched by the addition of sat. NH₄Cl (aq.) (50 mL) at 0° C. The resulting mixture was extracted with EtOAc (3×20 mL). The combined organic layers were washed with sat. NaCl (aq.) (2×15 mL), dried over anhydrous Na₂SO₄, filtered and concentrated under reduced pressure to afford 4-chloro-1-isopropyl-5-(4,4,5,5-tetramethyl-1,3,2-dioxaborolan-2-yl)pyrazol e(2 g crude) as a yell ow liquid. The crude product was used in the next step directly without further purification. LCMS (ESI) m/z 270.13 [M+H].

### Step 3: 2-Chloro-8-({4-[1-Methyl-4-(Trifluoromethyl)Imidazol-2-Yl]Phenyl}Methyl)Pyrido[2,3-d]Pyrimidin-7-One

A solution of 2-chloro-8H-pyrido[2,3-d]pyrimidin-7-one (80 mg, 0.441 mmol, 1 equiv), 2-[4-(chloromethyl)phenyl]-1-methyl-4-(trifluoromethyl)imidazole (108.91 mg, 0.397 mmol, 0.9 equiv) in DMF (2 mL, 25.843 mmol, 58.66 equiv) was treated with 1,1,3,3-Tetramethylguanidine (76.12 mg, 0.661 mmol, 1.5 equiv) for overnight at room temperature under nitrogen atmosphere. The residue was purified by reverse flash chromatography (C18 silica gel column, eluting with 0% to 100% ACN/water (0.1% FA) in 10 min with UV detector (254 nm)) to afford 2-chloro-8-({4-[1-methyl-4-(trifluoromethyl)imidazo]-2-yl]phenyl}methyl)pyrido[2,3-d]pyrimidin-7-one (120 mg, 64.88%) as an off-white solid. LCMS (ESI) m/z 419.08 [M+H].

### Step 4 2-(4-Chloro-2-Isopropylpyrazol-3-Yl)-8-({4-[1-Methyl-4-(Trifluoromethyl)Imidazol-2-Yl]Phenyl}Methyl)Pyrido[2,3-d]Pyrimidin-7-One

A solution of 2-chloro-8-({4-[1-methyl-4-(trifluoromethyl)imidazol-2-yl]phenyl}methyl)pyrido[2,3-d]pyrimidin-7-one (40 mg, 0.095 mmol, 1 equiv), 4-chloro-1-isopropyl-5-(4,4,5,5-tetramethyl-1,3,2-dioxaborolan-2-yl)pyrazole (77.34 mg, 0.285 mmol, 3 equiv) in 1,4-dioxane (2 mL) and H₂O (0.40 mL, 22.137 mmol, 233.02 equiv) was treated with K₃PO₄ (50.56 mg, 0.237 mmol, 2.5 equiv), Pd(dppf)Cl₂ (13.94 mg, 0.019 mmol, 0.2 equiv) for 2 h at 90° C. under nitrogen atmosphere. The resulting mixture was filtered, the filter cake was washed with acetone (2×5 mL). The filtrate was concentrated under reduced pressure. The residue was purified by reverse flash chromatography (C18 silica gel column, eluting with 0% to 100% ACN/water (0.1% FA) in 15 min with UV detector (254 nm)) to afford to afford 2-(4-chloro-2-isopropylpyrazol-3-yl)-8-({4-[1-methyl-4-(trifluoromethyl)imidazo]-2-yl]phenyl}methyl)pyrido[2,3-d]pyrimidin-7-one (21.3 mg, 40.82%) as an orange solid. ¹H NMR (300 MHz, Methanol-d4) δ 9.22 (s, 1H), 8.12 (d, J=9.5 Hz, 1H), 7.68 (d, J=1.4 Hz, 1H), 7.60 (d, J=8.2 Hz, 3H), 7.53-7.41 (m, 2H), 6.92 (d, J=9.5 Hz, 1H), 5.84 (s, 2H), 5.46-5.37 (m, 1H), 3.75 (s, 3H), 1.41 (d, J=6.6 Hz, 6H). LCMS (ESI) m/z 528.20 [M+H].

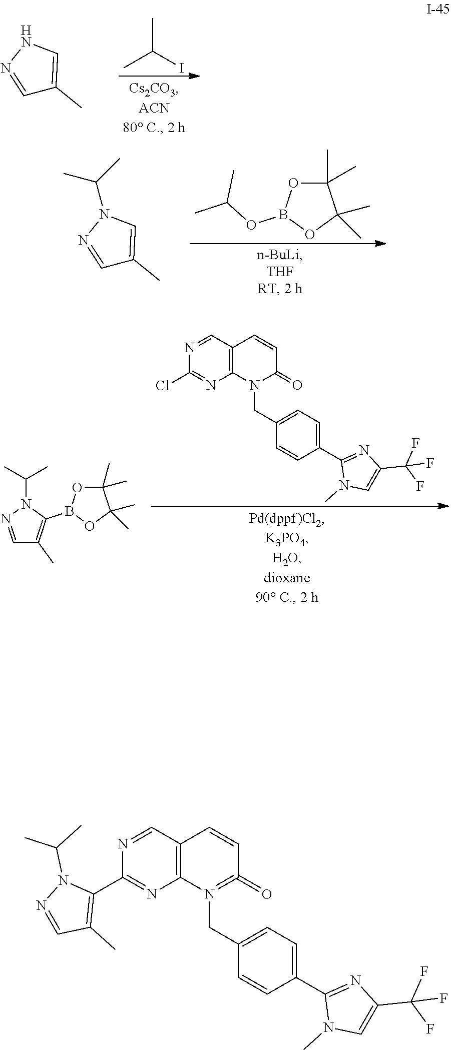

### Step 1 1-Isopropyl-4-Methylpyrazole

A solution of fomepizole (1 g, 12.179 mmol, 1 equiv), Cs₂CO₃ (7.94 g, 24.358 mmol, 2 equiv) in ACN (15 mL) was treated with 2-iodopropane (3.11 g, 18.268 mmol, 1.50 equiv) for 2 h at 80° C. under nitrogen atmosphere. The mixture was allowed to cool down to room temperature. The resulting mixture was filtered, the filter cake was washed with EtOAc (3×20 mL), filtered and concentrated under reduced pressure. The residue was purified by silica gel column chromatography, (eluting with PE/EA (5:1)) to afford 1-isopropyl-4-methylpyrazole (1.2 g, 79.34%) as a green liquid. LCMS (ESI) m/z 124.10 [M+H].

### Step 2 1-Isopropyl-4-Methyl-5-(4,4,5,5-Tetramethyl-1,3,2-Dioxaborolan-2-Yl)Pyrazole

To a stirred solution of 1-isopropyl-4-methylpyrazole (1.2 g, 9.663 mmol, 1 equiv) in THF (15 mL) were added n-BuLi (0.74 g, 11.596 mmol, 1.2 equiv., 2.5 M in hexane) dropwise at 0° C. under nitrogen atmosphere. The resulting mixture was stirred for 1 h at room temperature under nitrogen atmosphere. To the above mixture was added 2-isopropoxy-4,4,5,5-tetramethyl-1,3,2-dioxaborolane (4.64 mL, 11.596 mmol, 1.2 equiv) dropwise over at −78° C. The resulting mixture was stirred for additional 2 h at room temperature. The reaction was quenched by the addition of sat. NH₄Cl (aq.) (50 mL) at 0° C. The resulting mixture was extracted with EtOAc (3×mL). The combined organic layers were washed with sat. NaCl (aq.) (2×15 mL), dried over anhydrous Na₂SO₄, filtered and concentrated under reduced pressure to afford 1-isopropyl-4-methyl-5-(4,4,5,5-tetramethyl-1,3,2-dioxaborolan-2-yl)pyrazole (2 g crude) as a yellow liquid. The crude product was used in the next step directly without further purification. LCMS (ESI) m/z 250.19 [M+H].

### Step 3 2-(2-Isopropyl-4-Methylpyrazol-3-Yl)-8-({4-[1-Methyl-4-(Trifluoromethyl)Imidazol-2-Yl]Phenyl}Methyl)Pyrido[2,3-d]Pyrimidin-7-One

A solution of 2-chloro-8-({4-[1-methyl-4-(trifluoromethyl)imidazol-2-yl]phenyl}methyl)pyrido[2,3-d]pyrimidin-7-one (60 mg, 0.143 mmol, 1 equiv),1-isopropyl-4-methyl-5-(4,4,5,5-tetramethyl-1,3,2-dioxaborolan-2-yl)pyrazol e (107.26 mg, 0.429 mmol, 3 equiv) in 1,4-dioxane (2 mL) and H₂O (0.5 mL) was treated with K₃PO₄ (75.85 mg, 0.357 mmol, 2.5 equiv) and Pd(dppf)Cl₂ (20.92 mg, 0.029 mmol, 0.2 equiv) for 2 h at 90° C. under nitrogen atmosphere. The resulting mixture was filtered, the filter cake was washed with acetone (2×5 mL). The filtrate was concentrated under reduced pressure. The crude product (60 mg) was purified by Prep-HPLC with the following conditions (Column: XBridge Shield RP18 OBD Column, mm, 51 μm; Mobile Phase A: Water(10 mmol/L NH₄HCO₃), Mobile Phase B: ACN; Flow rate: 60 mL/min; Gradient: 45% B to 55% B in 8 min, 55% B; Wave Length: 254/220 nm; RT1(min): 6.48;) to afford 2-(2-isopropyl-4-methylpyrazol-3-yl)-8-({4-[1-methyl-4-(trifluoromethyl)imidazol-2-yl]phenyl}methyl)pyrido[2,3-d]pyrimidin-7-one (19.9 mg, 27.21%) as an off-white solid. ¹H NMR (300 MHz, DMSO-d₆) δ 9.28 (s, 1H), 8.16 (d, J=9.5 Hz, 1H), 7.91 (d, J=1.4 Hz, 1H), 7.65 (d, J=8.2 Hz, 2H), 7.41 (s, 1H), 7.30 (d, J=8.2 Hz, 2H), 6.88 (d, J=9.5 Hz, 1H), 5.65 (s, 2H), 5.44-5.35 (m, 1H), 3.73 (s, 3H), 2.17 (d, J=0.6 Hz, 3H), 1.24 (d, J=6.5 Hz, 6H). LCMS(ESI) m/z 508.30 [M+H].

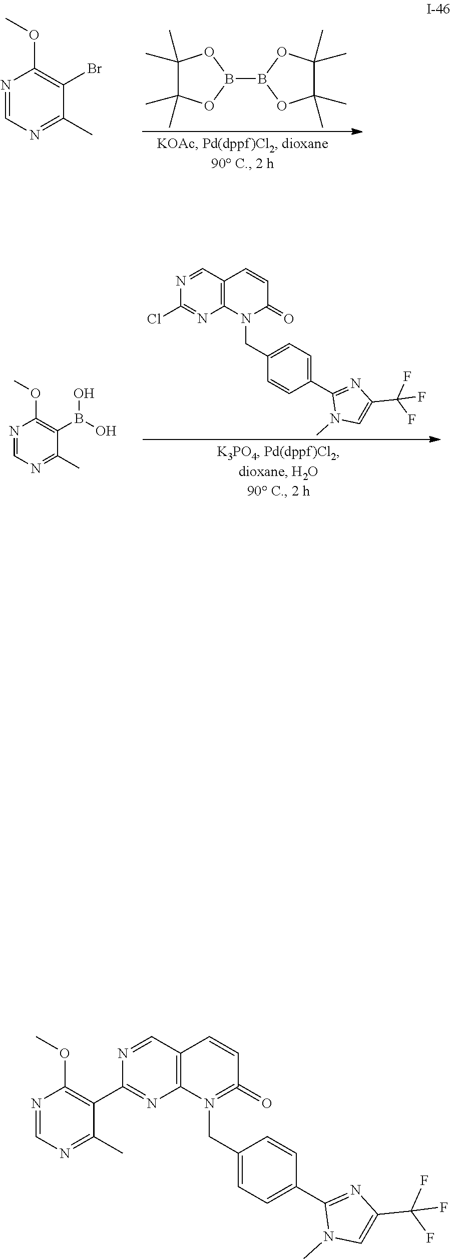

### Step 1: 4-Methoxy-6-Methylpyrimidin-5-Ylboronic Acid

To a stirred mixture of 5-bromo-4-methoxy-6-methylpyrimidine (300 mg, 1.478 mmol, 1 equiv) and bis(pinacolato)diboron (750.42 mg, 2.956 mmol, 2 equiv) in dioxane (6 mL, mmol, 47.93 equiv) were added KOAc (290.02 mg, 2.956 mmol, 2 equiv) and Pd(dppf)Cl₂ (108.11 mg, 0.148 mmol, 0.1 equiv) at room temperature under nitrogen atmosphere. The resulting mixture was stirred for 2 h at 90° C. under nitrogen atmosphere. The resulting mixture was extracted with EtOAc (3×20 mL). The combined organic layers were washed with brine (3×mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 4-methoxy-6-methylpyrimidin-5-ylboronic acid (150 mg, 60.44%) as a pink solid. LC-MS (ESI) m/z 169.1 [M+H]

### Step 2 2-(4-Methoxy-6-Methylpyrimidin-5-Yl)-8-({4-[1-Methyl-4-(Trifluoromethyl)Imidazol-2-Yl]Phenyl}Methyl)Pyrido[2,3-d]Pyrimidin-7-One

To a stirred mixture of 4-methoxy-6-methylpyrimidin-5-ylboronic acid (30.01 mg, mmol, 1.5 equiv) and 2-chloro-8-({4-[1-methyl-4-(trifluoromethyl)imidazo]-2-yl]phenyl}methyl)pyrido[2,3-d]pyrimidin-7-one (50 mg, 0.119 mmol, 1.00 equiv) in dioxane (0.8 mL, 9.443 mmol, 79.28 equiv) and H₂O (0.2 mL, 11.102 mmol, 93.21 equiv) were added K₃PO₄ (63.21 mg, 0.297 mmol, 2.5 equiv) and Pd(dppf)Cl₂ (17.43 mg, 0.024 mmol, 0.2 equiv) at room temperature under nitrogen atmosphere. The resulting mixture was stirred for 2 h at 90° C. under nitrogen atmosphere. The resulting mixture was extracted with EtOAc (3×5 mL). The combined organic layers were washed with brine (3×5 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 2-(4-methoxy-6-methylpyrimidin-5-yl)-8-({4-[1-methyl-4-(trifluoromethyl)imidazol-2-yl]phenyl]methyl)pyri do[2,3-d]pyrimidin-7-one (21.4 mg, as an off-white solid. ¹H NMR (400 MHz, DMSO-d6) δ 9.27 (s, 1H), 8.76 (s, 1H), 8.16 (d, J=9.6 Hz, 1H), 7.91 (d, J=1.4 Hz, 1H), 7.68-7.60 (m, 2H), 7.39 (d, J=8.3 Hz, 2H), 6.92 (d, J=9.5 Hz, 1H), 5.55 (s, 2H), 3.84 (s, 3H), 3.73 (s, 3H), 2.11 (s, 3H). LC-MS (ESI) m/z 508.2 [M+H].

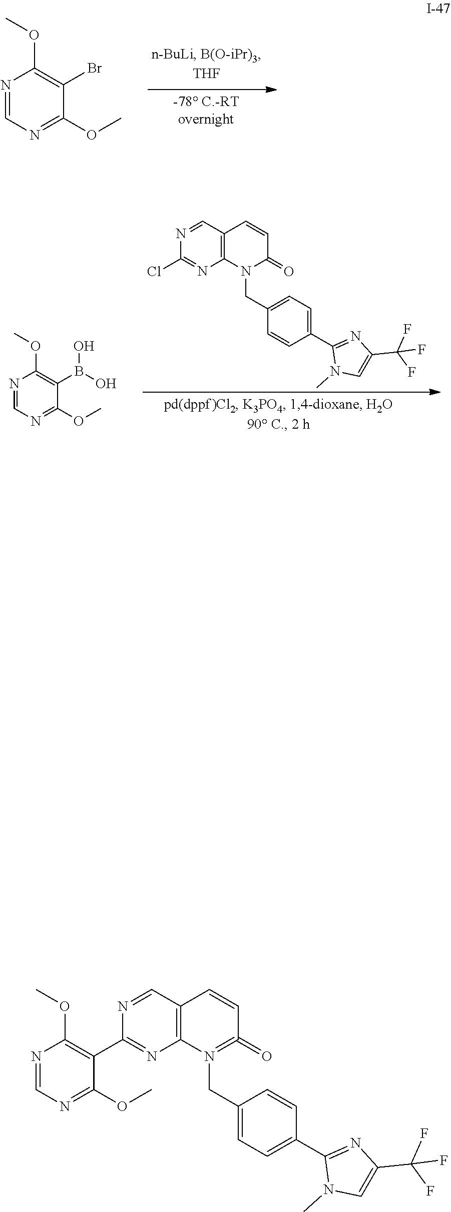

### Step 1: 4,6-Dimethoxypyrimidin-5-Ylboronic Acid

To a stirred 5-bromo-4,6-dimethoxypyrimidine (500 mg, 2.283 mmol, 1 equiv) in THF (10 mL, 45.660 mmol, 20 equiv) was added butyllithium (1.37 mL, 3.425 mmol, 1.5 equiv) dropwise at −78° C. under nitrogen atmosphere. The resulting mixture was stirred for at −78° C. under nitrogen atmosphere. To the above mixture was added triisopropyl borate (643.96 mg, 3.425 mmol, 1.5 equiv) dropwise over 1 min at −78° C. The resulting mixture was stirred for additional overnight at room temperature. The reaction was quenched with Ice/Salt at residue was basified to pH 8 with saturated NaHCO₃(aq.). The resulting mixture was extracted with EtOAc (3×5 mL). The combined organic layers were washed with brine (3×2 mL), dried over anhydrous Na₂SO₄, filtered and concentrated under reduced pressure. The residue was purified by silica gel column chromatography (eluting with PE/EA (5:1)) to afford 4,6-dimethoxypyrimidin-5-ylboronic acid (50 mg, 11.91%) as a light yellow solid. LC-MS (ESI) m/z 185.1

### Step 2 2-(4,6-Dimethoxypyrimidin-5-Yl)-8-({4-[1-Methyl-4-(Trifluoromethyl)Imidazol-2-Yl]Phenyl}Methyl)Pyrido[2,3-d]Pyrimidin-7-One

To a stirred mixture of 4,6-dimethoxypyrimidin-5-ylboronic acid (36.81 mg, 0.200 mmol, 1.5 equiv) and 2-chloro-8-({4-[1-methyl-4-(trifluoromethyl)imidazo]-2-yl]phenyl}methyl)pyrido[2,3-d]pyrimidin-7-one (56 mg, 0.133 mmol, 1.00 equiv) in 1,4-dioxane (2 mL, 40 equiv) and H₂O (0.5 mL, 10 equiv) were added K₃PO₄ (84.95 mg, 0.399 mmol, 3.0 equiv) and Pd(dppf)C12 (9.76 mg, 0.013 mmol, 0.1 equiv) at room temperature under nitrogen atmosphere. The resulting mixture was stirred for 2h at under nitrogen atmosphere. The resulting mixture was diluted with DMSO (1 mL). The crude product (50 mg) was purified by Prep-HPLC with the following conditions (Column: XBridge Shield RP18 OBD Column, 30*150 mm, 5 μm; Mobile Phase A: Water(0.1% NH₃.H₂O), Mobile Phase B: ACN; Flow rate: 60 mL/min; Gradient: 35% B to 50% B in 7 min, 50% B; Wave Length: 254/220 nm; RT1(min): 6.15;) to afford 2-(4,6-dimethoxypyrimidin-5-yl)-8-({4-[1-methyl-4-(trifluoromethyl)imidazo]-2-yl]phenyl}methyl)pyrido[2,3-d]pyrimidin-7-one (9.6 mg, 13.73%) as a white solid. ¹H NMR (400 MHz, DMSO-d6) δ 9.23 (s, 1H), 8.64 (s, 1H), 8.11 (d, J=9.5 Hz, 1H), 7.92 (d, J=1.4 Hz, 1H), 7.65 (d, J=8.2 Hz, 2H), 7.46 (d, J=8.2 Hz, 2H), 6.87 (d, J=9.5 Hz, 1H), 5.53 (s, 2H), 3.86 (s, 5H), 3.74 (s, 3H). LC-MS (ESI) m/z 524.25 [M+H]

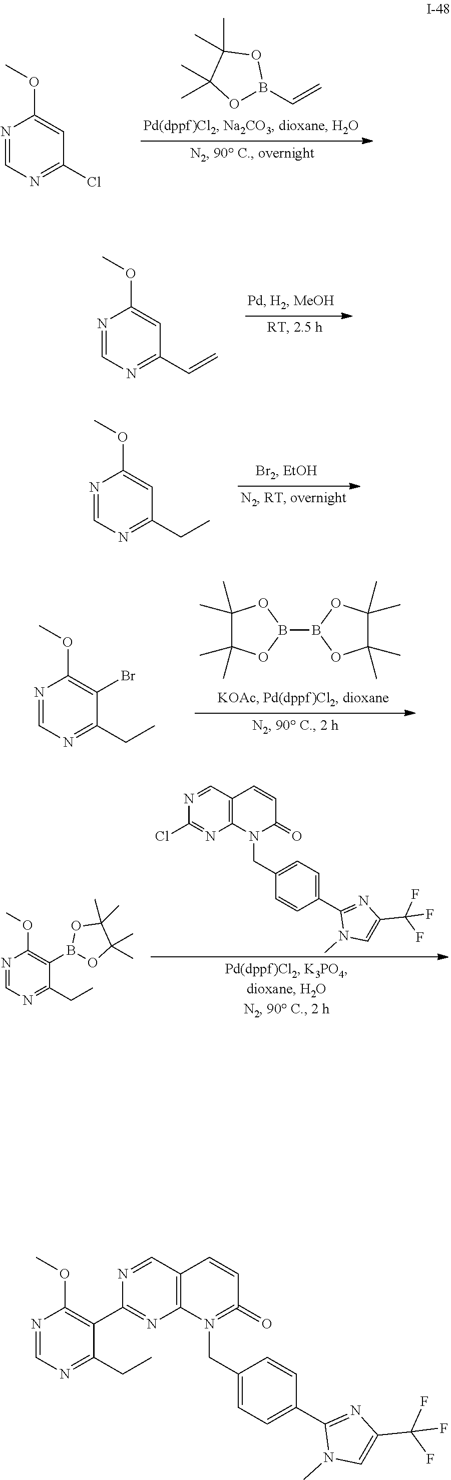

### Step 1: 4-Ethenyl-6-Methoxypyrimidine

To a stirred solution of 4-chloro-6-methoxypyrimidine (2 g, 13.835 mmol, 1 equiv) and 2-ethenyl-4,4,5,5-tetramethyl-1,3,2-dioxaborolane (3.20 g, 20.753 mmol, 1.5 equiv) in dioxane (20 mL) and H₂O (4 mL) were added Na₂CO₃ (2.93 g, 27.670 mmol, 2 equiv) and Pd(dppf)Cl₂ (2.02 g, 2.767 mmol, 0.2 equiv) at room temperature. The resulting mixture was stirred for overnight at 90° C. under nitrogen atmosphere. The resulting mixture was filtered, the filter cake was washed with THF (2×5 mL). The filtrate was concentrated under reduced pressure. The residue was purified by silica gel column chromatography, eluted with PE/EA (12:1) to afford 4-ethenyl-6-methoxypyrimidine (1.3954 g, 74.08%) as a colorless oil. LC-MS (ESI) m/z 137.1 [M+H]

### Step 2: 4-Ethyl-6-Methoxypyrimidine

To a solution of 4-ethenyl-6-methoxypyrimidine (1.39 g, 10.209 mmol, 1 equiv) in 15 mL MeOH was added Pd/C (10%, 2 g) under nitrogen atmosphere in a 50 mL round-bottom flask. The mixture was hydrogenated at room temperature for 2.5 h under hydrogen atmosphere, filtered through a Celite pad and concentrated under reduced pressure at 0° C. to afford 4-ethyl-6-methoxypyrimidine (830.9 mg, Crude) as a colorless oil. The crude product was used in the next step directly without further purification. LC-MS (ESI) m/z 139.1 [M+H]

### Step 3: 5-Bromo-4-Ethyl-6-Methoxypyrimidine

To a stirred solution of 4-ethyl-6-methoxypyrimidine (830.9 mg, 6.014 mmol, 1 equiv) in EtOH (10 mL) was added Br₂ (2.88 g, 18.042 mmol, 3 equiv) dropwise at 0° C. under nitrogen atmosphere. The resulting mixture was stirred for overnight at room temperature under nitrogen atmosphere. The resulting mixture was concentrated under reduced pressure at 0° C. The residue was purified by reversed-phase flash chromatography (C18 silica gel column, eluting with 0% to 100% MeCN/water (0.1% FA) in 10 min with UV detector (254 nm)) to afford 5-bromo-4-ethyl-6-methoxypyrimidine (406.8 mg, 31.16%) as a yellow oil. LC-MS (ESI) m/z 218.

### Step 4: 4-Ethyl-6-Methoxy-5-(4,4,5,5-Tetramethyl-1,3,2-Dioxaborolan-2-Yl)Pyrimidine

To a stirred solution of 5-bromo-4-ethyl-6-methoxypyrimidine (356 mg, 1.640 mmol, 1 equiv) and bis(pinacolato)diboron (832.95 mg, 3.280 mmol, 2 equiv) in dioxane (6 mL) were added KOAc (482.87 mg, 4.920 mmol, 3 equiv) and Pd(dppf)Cl₂ (180.01 mg, 0.246 mmol, equiv) at room temperature. The resulting mixture was stirred for 2 h at 90° C. under nitrogen atmosphere. The resulting mixture was filtered, the filter cake was washed with THF (2×5 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford 4-ethyl-6-methoxy-5-(4,4,5,5-tetramethyl-1,3,2-dioxaborolan-2-yl)pyrimidine (45.8 mg, 10.57%) as a light yellow oil. LC-MS (ESI) m/z 265.1 [M+H]

### Step 5: 2-(4-Ethyl-6-Methoxypyrimidin-5-Yl)-8-({4-[1-Methyl-4-(Trifluoromethyl)Imidazol-2-Yl]Phenyl}Methyl)Pyrido[2,3-d]Pyrimidin-7-One

To a stirred solution of 4-ethyl-6-methoxy-5-(4,4,5,5-tetramethyl-1,3,2-dioxaborolan-2-yl)pyrimidine (26.43 mg, 0.101 mmol, 1.5 equiv) and 2-chloro-8-({4-[1-methyl-4-(trifluoromethyl)imidazo]-2-yl]phenyl}methyl)pyrido[2,3-d]pyrimidin-7-one (28 mg, 0.067 mmol, 1.00 equiv) in dioxane (2 mL) and H₂O (0.2 mL) were added K₃PO₄ (35.40 mg, 0.168 mmol, 2.5 equiv) and Pd(dppf)C12 (9.76 mg, 0.013 mmol, 0.2 equiv) at room temperature. The resulting mixture was stirred for 2 h at 90° C. under nitrogen atmosphere. The resulting mixture was filtered, the filter cake was washed with THF (2×3 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford 2-(4-ethyl-6-methoxypyrimidin-5-yl)-8-({4-[1-methyl-4-(trifluoromethyl)imidazo]-2-yl]phenyl}methyl)pyrido[2,3-d]pyrimidin-7-one (5.0 mg, 14.37%) as an off-white solid. ¹H NMR (400 MHz, DMSO-d6) δ 9.28 (s, 1H), 8.81 (s, 1H), 8.16 (d, J=9.5 Hz, 1H), 7.96-7.84 (m, 1H), 7.68-7.56 (m, 2H), 7.36 (d, J=8.2 Hz, 2H), 6.92 (d, J=9.5 Hz, 1H), 5.56 (s, 2H), 3.84 (s, 3H), 3.74 (s, 3H), 2.38 (q, J=7.5 Hz, 2H), 0.95 (t, J=7.5 Hz, 3H). LC-MS (ESI) m/z 522.15 [M+H]

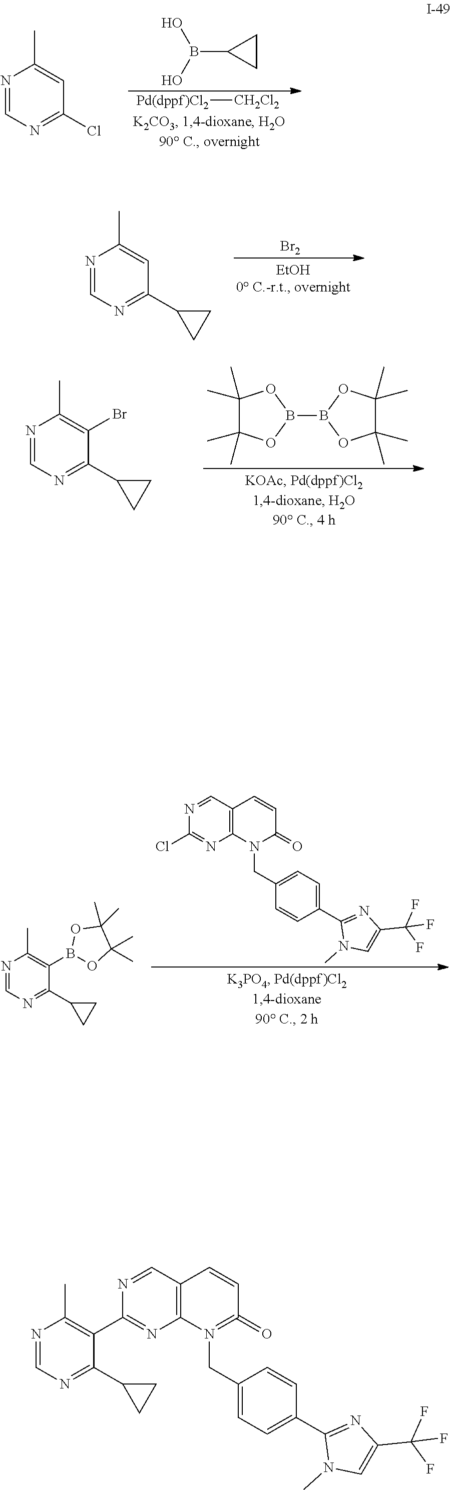

### Step 1: 4-Cyclopropyl-6-Methylpyrimidine

To a stirred solution of 4-chloro-6-methylpyrimidine (1.93 g, 15.012 mmol, 1 equiv) and cyclopropylboronic acid (3868.71 mg, 45.036 mmol, 3 equiv) in 1,4-dioxane (9 mL, 408.603 mmol, 56.48 equiv) were added H₂O (1.87 mL, 103.733 mmol, 6.91 equiv) and K₂CO₃ (4149.59 mg, 30.024 mmol, 2 equiv) at room temperature under nitrogen atmosphere. To the above mixture was added Pd(dppf)C12′CH₂Cl₂ (1834.42 mg, 2.252 mmol, 0.15 equiv) at room temperature. The resulting mixture was stirred for additional overnight at 90° C. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% FA) with UV detector (254 nm)) to yield 4-cyclopropyl-6-methylpyrimidine (840.5 mg, 41.72%) as an orange oil.LC-MS (ESI) m/z 135 [M+H]

### Step 2 5-Bromo-4-Cyclopropyl-6-Methylpyrimidine

To a stirred solution of 4-cyclopropyl-6-methylpyrimidine (840.5 mg, 6.264 mmol, 1 equiv) in EtOH (1.2 mL, 20.656 mmol, 3.30 equiv) were added Br₂ (1.03 mL, 18.792 mmol, 3 equiv) dropwise at 0° C. under nitrogen atmosphere. The resulting mixture was stirred for additional overnight at room temperature. The mixture was basified to pH 7 with saturated NaHCO₃(aq.). The resulting mixture was extracted with EtOAc (3×20 mL). The combined organic layers were washed with brine (3×10 mL), dried over anhydrous Na₂SO₄, filtered and concentrated under reduced pressure to afford 5-bromo-4-cyclopropyl-6-methylpyrimidine (682.9 mg, 51.17%) as an orange solid. The crude product mixture was used in the next step directly without further purification. LC-MS (ESI) m/z 213 [M+H]

### Step 3: 4-Cyclopropyl-6-Methyl-5-(4,4,5,5-Tetramethyl-1,3,2-Dioxaborolan-2-Yl)Pyrimidine

To a stirred solution of 5-bromo-4-cyclopropyl-6-methylpyrimidine (200 mg, 0.939 mmol, 1 equiv) and bis(pinacolato)diboron (476.71 mg, 1.878 mmol, 2 equiv) in dioxane (4 mL, 47.216 mmol, 50.30 equiv) were added KOAc (276.36 mg, 2.817 mmol, 3 equiv) and Pd(dppf)Cl₂ (103.02 mg, 0.141 mmol, 0.15 equiv) at room temperature under nitrogen atmosphere. The resulting mixture was stirred for additional 4 h at 90° C. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% TFA) with UV detector (254 nm)) to yield 4-cyclopropyl-6-methyl-5-(4,4,5,5-tetramethyl-1,3,2-dioxaborolan-2-yl)pyrimidine (113.6 mg, 46.52%) as a brown oil. LC-MS (ESI) m/z 261 [M+H]

### Step 4: 2-(4-Cyclopropyl-6-Methylpyrimidin-5-Yl)-8-({4-[1-Methyl-4-(Trifluoromethyl)Imidazol-2-Yl]Phenyl}Methyl)Pyrido[2,3-d]Pyrimidin-7-One

To a stirred solution of 4-cyclopropyl-6-methyl-5-(4,4,5,5-tetramethyl-1,3,2-dioxaborolan-2-yl)pyrimidine (113.60 mg, 0.437 mmol, 1.5 equiv) and 2-chloro-8-({4-[1-methyl-4-(trifluoromethyl)imidazo]-2-yl]phenyl}methyl)pyrido[2,3-d]pyrimidin-7-one (122.21 mg, mmol, 1.00 equiv) in dioxane (2 mL, 23.608 mmol, 54.06 equiv) were added K₃PO₄ (154.49 mg, 0.728 mmol, 2.5 equiv) and H₂O (0.4 mL, 22.204 mmol, 50.85 equiv) at room temperature under nitrogen atmosphere. To the above mixture was added Pd(dppf)Cl₂ (42.60 mg, 0.058 mmol, equiv) at room temperature. The resulting mixture was stirred for additional 2 h at 90° C. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% TFA) with UV detector (254 nm)) to yield 2-(4-cyclopropyl-6-methylpyrimidin-5-yl)-8-({4-[1-methyl-4-(trifluoromethyl)imidazo]-2-yl]phenyl}methyl)pyrido[2,3-d]pyrimidin-7-one (57 mg, 37.27%) as a yellow solid. ¹H NMR (400 MHz, DMSO-d6) δ 9.34 (s, 1H), 8.88 (s, 1H), 8.21 (d, J=9.6 Hz, 1H), 7.91 (d, J=1.5 Hz, 1H), 7.62 (d, J=8.1 Hz, 2H), 7.34 (d, J=8.1 Hz, 2H), 6.95 (d, J=9.5 Hz, 1H), 5.59 (s, 2H), 3.73 (s, 3H), 2.11 (s, 3H), 1.72 (s, 1H), 1.01 (s, 2H), 0.78 (dd, J=8.1, 3.3 Hz, 2H). LC-MS (ESI) m/z 518 [M+H].

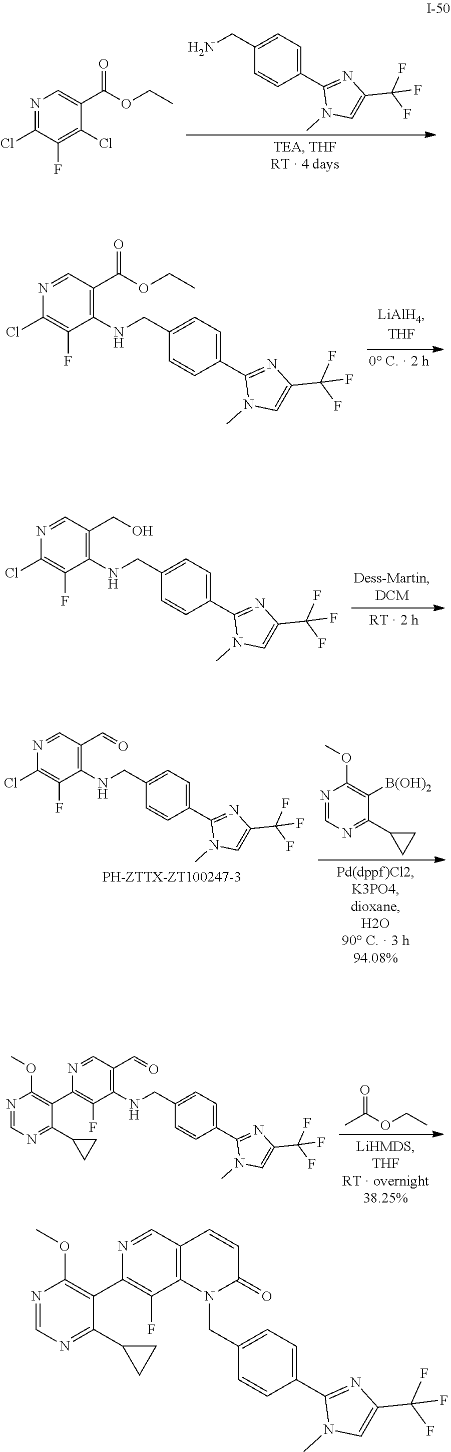

### Step 1 Ethyl 6-Chloro-5-Fluoro-4-1({4-[1-Methyl-4-(Trifluoromethyl)Imidazol-2-Yl]Phenyl}Methyl)Amino]Pyridine-3-Carboxylate

To a stirred solution of ethyl 4,6-dichloro-5-fluoropyridine-3-carboxylate (300 mg, 1.260 mmol, 1 equiv) and 1-{4-[1-methyl-4-(trifluoromethyl)imidazo]-2-yl]phenyl}methanamine (289.51 mg, 1.134 mmol, 0.9 equiv) in THF (6 mL) was added TEA (382.60 mg, 3.780 mmol, 3 equiv) at room temperature under nitrogen atmosphere. The resulting mixture was stirred for 4 days at room temperature under nitrogen atmosphere. The residue was purified by reverse flash chromatography (C18 silica gel column, eluting with 10% to 100% MeCN/water (0.1% NH₃H₂O) in 10 min with UV detector (254 nm)) to afford ethyl 6-chloro-5-fluoro-44({4-[1-methyl-4-(trifluoromethyl)imidazo]-2-yl]phenyl}methyl)amino]pyridine-3-carboxylate (270 mg, 46.90%) as a white solid. LC-MS (ESI) m/z 457 [M+H].

### Step 2 {6-Chloro-5-Fluoro-4-1({4-[1-Methyl-4-(Trifluoromethyl)Imidazol-2-Yl]Phenyl}Methyl)Amino]Pyridin-3-Yl}Methanol

To a stirred solution of ethyl 6-chloro-5-fluoro-4-[({4-[1-methyl-4-(trifluoromethyl)imidazo]-2-yl]phenyl}methyl)amino]pyridine-3-carboxylate (270 mg, 0.591 mmol, 1 equiv) in THF (5 mL) was added LiAlH₄ (24.67 mg, 0.650 mmol, 1.1 equiv) in portions at 0° C. under nitrogen atmosphere. The resulting mixture was stirred for 2 h at 0° C. under nitrogen atmosphere. The reaction was quenched by the addition of Sodium sulfate decahydrate at 0° C. The residue was purified by reverse flash chromatography (C18 silica gel column, eluting with 10% to 100% MeCN/water (0.1% NH₃H₂O) in 10 min with UV detector (254 nm)) to afford {6-chloro-5-fluoro-4-[({4-[1-methyl-4-(trifluoromethyl)imidazo]-2-yl]phenyl}methyl)amino]pyridin-3-yl}methanol (220 mg, 89.74%) as a white solid. LC-MS (ESI) m/z 415 [M+H].

### Step 3: 6-Chloro-5-Fluoro-4-1({4-[1-Methyl-4-(Trifluoromethyl)Imidazol-2-Yl]Phenyl}Methyl)Amino]Pyridine-3-Carbaldehyde

To a stirred solution of {6-chloro-5-fluoro-4-[({4-[1-methyl-4-(trifluoromethyl)imidazol-2-yl]phenyl methyl)amino]pyridin-3-yl}methanol (210 mg, 0.506 mmol, 1 equiv) in DCM (4 mL) was added 1,1-bis(acetyloxy)-3-oxo-3H-11\hat{ }[5],2-benziodaoxol-1-yl acetate (429.47 mg, 1.012 mmol, 2 equiv) at room temperature under nitrogen atmosphere. The resulting mixture was stirred for 2 h at room temperature under nitrogen atmosphere. The residue was purified by reverse flash chromatography (C18 silica gel column, eluting with 10% to 100% MeCN/water (0.1% NH₃.H₂O) in 10 min with UV detector (254 nm)) to afford 6-chloro-5-fluoro-44({4-[1-methyl-4-(trifluoromethyl)imidazo]-2-yl]phenyl methyl)amino]pyridine-3-carbaldehyde (100 mg, 47.85%) as a white solid. LC-MS (ESI) m/z 413 [M+H].

### Step 4: 6-(4-Cyclopropyl-6-Methoxypyrimidin-5-Yl)-5-Fluoro-4-1({4-[1-Methyl-4-(Trifluoromethyl)Imidazol-2-Yl]Phenyl}Methyl)Amino]Pyridine-3-Carbaldehyde

To a stirred solution of 6-chloro-5-fluoro-4-[({4-[1-methyl-4-(trifluoromethyl)imidazo]-2-yl]phenyl}methyl)amino]pyridine-3-carbaldehyde (50 mg, 0.121 mmol, 1 equiv) and 4-cyclopropyl-6-methoxypyrimidin-5-ylboronic acid (28.20 mg, 0.145 mmol, 1.2 equiv) in dioxane (1 mL) was added K₃PO₄ (64.28 mg, 0.302 mmol, 2.5 equiv) and Pd(dppf)C12 (8.86 mg, 0.012 mmol, 0.1 equiv), H₂O (0.5 mL) at room temperature under nitrogen atmosphere. The resulting mixture was stirred for 3 h at 90° C. under nitrogen atmosphere. The residue was purified by reverse flash chromatography (C18 silica gel column, eluting with 10% to 100% MeCN/water (0.1% NH₃.H₂O) in 10 min with UV detector (254 nm)) to afford 6-(4-cyclopropyl-6-methoxypyrimidin-5-yl)-5-fluoro-44({4-[1-methyl-4-(trifluoromethyl)imidazo]-2-yl]phenyl}methyl)amino]pyridine-3-carbaldehyde (60 mg, 94.08%) as a yellow oil. LC-MS (ESI) m/z 527 [M+H].
Step 5: 7-(4-cyclopropyl-6-methoxypyrimidin-5-yl)-8-fluoro-1-({4-[1-methyl-4-(trifluoromethyl)imidazol-2-yl]phenyl}methyl)-1,6-naphthyridin-2-one

A solution of ethyl acetate (117.14 mg, 1.330 mmol, 10 equiv) was treated with LiHMDS (0.9 mL, 0.950 mmol, 10 equiv) for 40 min at −78° C. under nitrogen atmosphere followed by the addition of 6-(4-cyclopropyl-6-methoxypyrimidin-5-yl)-5-fluoro-44({4-[1-methyl-4-(trifluoromethyl)imidazo]-2-yl]phenyl methyl)amino]pyri dine-3-carb aldehyde (70 mg, mmol, 1 equiv) at −78° C. The resulting mixture was stirred for overnight at room temperature under nitrogen atmosphere. The residue was purified by reverse flash chromatography (C18 silica gel column, eluting with 10% to 100% MeCN/water (0.1% NH₃.H₂O) in 10 min with UV detector (254 nm)) to afford 7-(4-cyclopropyl-6-methoxypyrimidin-5-yl)-8-fluoro-1-({4-[1-methyl-4-(trifluoromethyl)imidazol-2-yl]phenyl}methyl)-1,6-naphthyridin-2-one (28 mg, 38.25%) as a white solid. ¹H NMR (400 MHz, DMSO-d6) δ 8.92 (s, 1H), 8.67 (s, 1H), 8.23 (dd, J=9.6, 1.9 Hz, 1H), 7.94-7.89 (m, 1H), 7.69-7.63 (m, 2H), 7.26 (d, J=8.1 Hz, 2H), 6.97 (d, J=9.6 Hz, 1H), 5.65 (s, 2H), 3.74 (d, J=4.6 Hz, 6H), 1.61 (s, 1H), 1.07 (d, J=6.9 Hz, 1H), 0.88 (dt, J=13.4, 6.1 Hz, 2H), 0.72 (s, 1H). LC-MS (ESI) m/z 551.10 [M+H].

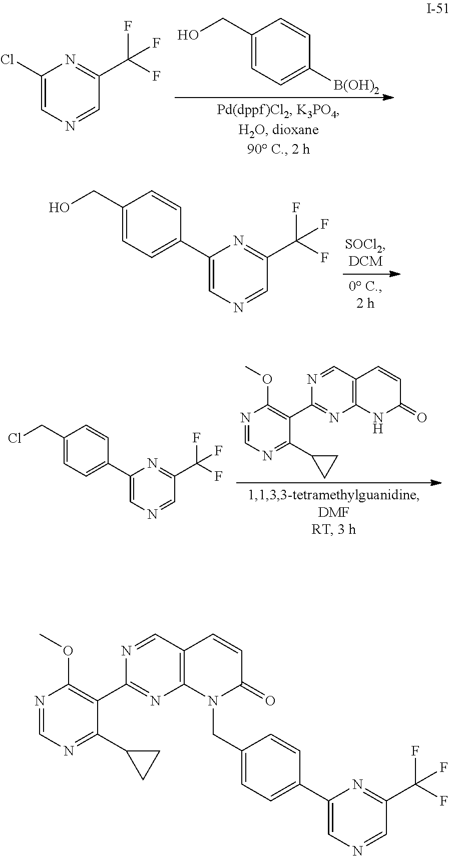

### Step 1: {4-[6-(Trifluoromethyl)Pyrazin-2-Yl]Phenyl}Methanol

To a stirred solution of 2-chloro-6-(trifluoromethyl)pyrazine (250 mg, 1.370 mmol, 1 equiv) and 4-(hydroxymethyl)phenylboronic acid (249.76 mg, 1.644 mmol, 1.2 equiv) in dioxane (3 mL) was added K₃PO₄ (726.82 mg, 3.425 mmol, 2.5 equiv), Pd(dppf)Cl₂ (100.22 mg, 0.137 mmol, 0.1 equiv) and H₂O (1 mL) at room temperature under air atmosphere. The resulting mixture was stirred for 2 h at 90° C. under nitrogen atmosphere. The residue was purified by reverse flash chromatography (C18 silica gel column, eluting with 10% to 100% MeCN/water (0.1% FA) in 10 min with UV detector (254 nm)) to afford {4-[6-(trifluoromethyl)pyrazin-2-yl]phenyl}methanol (300 mg, 86.16%) as a white solid. LC-MS (ESI) m/z 255 [M+H].

### Step 2: 2-[4-(Chloromethyl)Phenyl]-6-(Trifluoromethyl)Pyrazine

To a stirred solution of {4-[6-(trifluoromethyl)pyrazin-2-yl]phenyl}methanol (100 mg, 0.393 mmol, 1 equiv) in DCM (2 mL) was added SOCl₂ (1 mL, 13.786 mmol, 35.05 equiv) at 0° C. under air atmosphere. The resulting mixture was stirred for 2 h at 0° C. under nitrogen atmosphere. The resulting mixture was concentrated under reduced pressure. The residue was purified by reverse flash chromatography (C18 silica gel column, eluting with 10% to 100% MeCN/water (0.1% FA) in 10 min with UV detector (254 nm)) to afford 2-[4-(chloromethyl)phenyl]-6-(trifluoromethyl)pyrazine (90 mg, 83.91%) as a yellow solid. LC-MS (ESI) m/z 273 [M+H].

### Step 3: 2-(4-Cyclopropyl-6-Methoxypyrimidin-5-Yl)-8-({4-[6-(Trifluoromethyl)Pyrazin-2-Yl]Phenyl}Methyl)Pyrido[2,3-d]Pyrimidin-7-One

To a stirred solution of 2-[4-(chloromethyl)phenyl]-6-(trifluoromethyl)pyrazine (50 mg, 0.183 mmol, 1 equiv) and 2-(4-cyclopropyl-6-methoxypyrimidin-5-yl)-8H-pyrido[2,3-d]pyrimidin-7-one (64.98 mg, 0.220 mmol, 1.2 equiv) in DMF (1 mL) was added N,N,N′,N′-tetramethylguanidine (31.68 mg, 0.274 mmol, 1.5 equiv) at room temperature under air atmosphere. The resulting mixture was stirred for 3 h at room temperature under nitrogen atmosphere. The residue was purified by reverse flash chromatography (C18 silica gel column, eluting with 10% to 100% MeCN/water (0.1% FA) in 10 min with UV detector (254 nm)) to afford 2-(4-cyclopropyl-6-methoxypyrimidin-5-yl)-8-({4-[6-(trifluoromethyl)pyrazin-2-yl]phenyl}methyl)pyrido[2,3-d]pyrimidin-7-one (82 mg, 84.13%) as a white solid. ¹H NMR (400 MHz, DMSO-d6) δ 9.56 (s, 1H), 9.29 (s, 1H), 9.13 (s, 1H), 8.68 (s, 1H), 8.17 (d, J=9.5 Hz, 1H), 8.10 (d, J=8.1 Hz, 2H), 7.46 (d, J=8.1 Hz, 2H), 6.91 (d, J=9.5 Hz, 1H), 5.59 (s, 2H), 3.81 (s, 3H), 1.70 (dd, J=8.3, 4.1 Hz, 1H), 1.00 (s, 2H), 0.75 (dd, J=8.1, 3.3 Hz, 2H). LC-MS (ESI) m/z 532.20 [M+H]. 1-52

### Step 1: 4-Chloro-6-Ethenylpyrimidine

A solution of 4,6-dichloropyrimidine (500 mg, 3.356 mmol, 1 equiv) in dioxane (5 mL, 59.020 mmol, 17.58 equiv) and H₂O (0.5 mL, 27.755 mmol, 8.27 equiv) was treated with 2-ethenyl-4,4,5,5-tetramethyl-1,3,2-dioxaborolane (516.95 mg, 3.356 mmol, 1 equiv), Na₂CO₃ (2845.89 mg, 26.852 mmol, 2 equiv) and Pd(dppf)Cl₂ (245.59 mg, 0.336 mmol, 0.1 equiv) for overnight at 90° C. under nitrogen atmosphere. The resulting mixture was filtered, the filter cake was washed with EtOAc (3×10 mL). The filtrate was concentrated under reduced pressure. The residue was purified by silica gel column chromatography, (eluting with PE/EA (1:1)) to afford 4-chloro-6-ethenylpyrimidine (140 mg, 29.67%) as a yellow liquid. LCMS (ESI) m/z 141.01 [M+H].

### Step 2: 4-Cyclopropyl-6-Ethenylpyrimidine

A solution of 4-chloro-6-ethenylpyrimidine (450 mg, 3.201 mmol, 1 equiv) in dioxane (10 mL, 118.040 mmol, 36.87 equiv) and H₂O (1 mL, 55.509 mmol, 17.34 equiv) was treated with cyclopropylboronic acid (549.98 mg, 6.402 mmol, 2 equiv), K₂CO₃ (884.86 mg, 6.402 mmol, 2 equiv) and Pd(dppf)C12CH₂Cl₂ (260.78 mg, 0.320 mmol, 0.10 equiv) for overnight at under nitrogen atmosphere. The resulting mixture was filtered, the filter cake was washed with MeOH (3×10 mL). The filtrate was concentrated under reduced pressure. The residue was purified by silica gel column chromatography, (eluting with PE/EA (9:1)) to afford 4-cyclopropyl-6-ethenylpyrimidine (160 mg, 34.19%) as a yellow liquid. LCMS (ESI) m/z 147.08 [M+H].

### Step 3: 4-Cyclopropyl-6-Ethylpyrimidine

A solution of 4-cyclopropyl-6-ethenylpyrimidine (160 mg, 1.094 mmol, 1 equiv) in methanol (2 mL) was treated with Pd/C (80 mg, 0.752 mmol, 0.69 equiv) for 2 h at room temperature under hydrogen atmosphere. The resulting mixture was filtered, the filter cake was washed with MeOH (3×10 mL). The filtrate was concentrated under reduced pressure to afford 4-cyclopropyl-6-ethylpyrimidine (140 mg, crude) as an orange liquid crude. LCMS (ESI) m/z 149.10 [M+H].

### Step 4: 5-Bromo-4-Cyclopropyl-6-Ethylpyrimidine

A solution of 4-cyclopropyl-6-ethylpyrimidine (140 mg, 0.945 mmol, 1 equiv) in methanol (2 mL) was added Br2 (0.15 mL, 2.835 mmol, 3 equiv) in portions at 0° C. The resulting mixture was stirred for additional overnight at room temperature. The reaction was quenched by the addition of sat. NaHCO₃(aq.) (5 mL) at 0° C. The resulting mixture was extracted with EtOAc (3×5 mL). The combined organic layers were washed with EtOAc (2×5 mL), dried over anhydrous Na₂SO₄, filtered and concentrated under reduced pressure. The residue was purified by reversed-phase flash chromatography (C18 silica gel column, eluting with 10% to 100% MeCN/water (0.1% FA) in 10 min with UV detector (254 nm)). This resulted in 5-bromo-4-cyclopropyl-6-ethylpyrimidine (140 mg, 65.26%) as an orange liquid. LCMS (ESI) m/z 227.01 [M+H].

### Step 5: 4-Cyclopropyl-6-Ethyl-5-(4,4,5,5-Tetramethyl-1,3,2-Dioxaborolan-2-Yl)Pyrimidine

A solution of 5-bromo-4-cyclopropyl-6-ethylpyrimidine (140 mg, 0.616 mmol, 1 equiv) in dioxane (2 mL, 23.608 mmol, 38.30 equiv) was treated with bis(pinacolato)diboron (313.09 mg, 1.232 mmol, 2 equiv), KOAc (121.00 mg, 1.232 mmol, 2 equiv) and Pd(dppf)Cl₂ (67.66 mg, 0.092 mmol, 0.15 equiv) for 2 h at 90° C. under nitrogen atmosphere. The resulting mixture was filtered, the filter cake was washed with MeOH (3×5 mL). The filtrate was concentrated under reduced pressure. The residue was purified by reversed-phase flash chromatography (C18 silica gel column, eluting with 10% to 50% MeCN/water (0.1% FA) in 10 min with UV detector (254 nm)) to afford 4-cyclopropyl-6-ethyl-5-(4,4,5,5-tetramethyl-1,3,2-dioxaborolan-2-yl)pyrimidine (130 mg, 76.92%) as a yellow solid. LCMS (ESI) m/z 193.11 [M+H].

### Step 6: 2-(4-Cyclopropyl-6-Ethylpyrimidin-5-Yl)-8-({4-[1-Methyl-4-(Trifluoromethyl)Imidazol-2-Yl]Phenyl}Methyl)Pyrido[2,3-d]Pyrimidin-7-One

A solution of 4-cyclopropyl-6-ethyl-5-(4,4,5,5-tetramethyl-1,3,2-dioxaborolan-2-yl)pyrimidine (38 mg, 0.139 mmol, 1.2 equiv) in dioxane (2 mL, 23.608 mmol, 170.33 equiv) and H₂O (0.2 mg, 0.011 mmol, 0.08 equiv) was treated with 2-chloro-8-({4-[1-methyl-4-(trifluoromethyl)imidazo]-2-yl]phenyl}methyl)pyrido[2,3-d]pyrimidin-7-one (48.49 mg, 0.116 mmol, 1 equiv) and Pd(dppf)Cl₂ (33.81 mg, 0.046 mmol, 0.4 equiv) for 2 h at 90° C. under nitrogen atmosphere. The resulting mixture was filtered, the filter cake was washed with MeOH (3×5 mL). The residue was purified by reverse flash chromatography (C18 silica gel column, eluting with 10% to 50% MeCN/water (0.1% FA) in 10 min with UV detector (254 nm)) to afford 2-(4-cyclopropyl-6-ethylpyrimidin-5-yl)-8-({4-[1-methyl-4-(trifluoromethyl)imidazo]-2-yl]phenyl}methyl)pyrido[2,3-d]pyrimidin-7-one (6 mg, 9.40%) as a yellow solid. ¹H NMR (400 MHz, DMSO-d6) δ 9.33 (s, 1H), 8.94 (s, 1H), 8.21 (d, J=9.5 Hz, 1H), 7.91 (d, J=1.4 Hz, 1H), 7.61 (d, J=8.2 Hz, 2H), 7.32 (d, J=8.0 Hz, 2H), 6.94 (d, J=9.5 Hz, 1H), 5.59 (s, 2H), 3.73 (s, 3H), 2.38 (q, J=7.5 Hz, 2H), 1.69-1.64 (m, 1H), 1.02 (d, J=4.0 Hz, 2H), 0.94 (t, J=7.5 Hz, 3H), 0.79 (dd, J=7.9, 3.4 Hz, 2H). LCMS (ESI) m/z 532.30 [M+H].

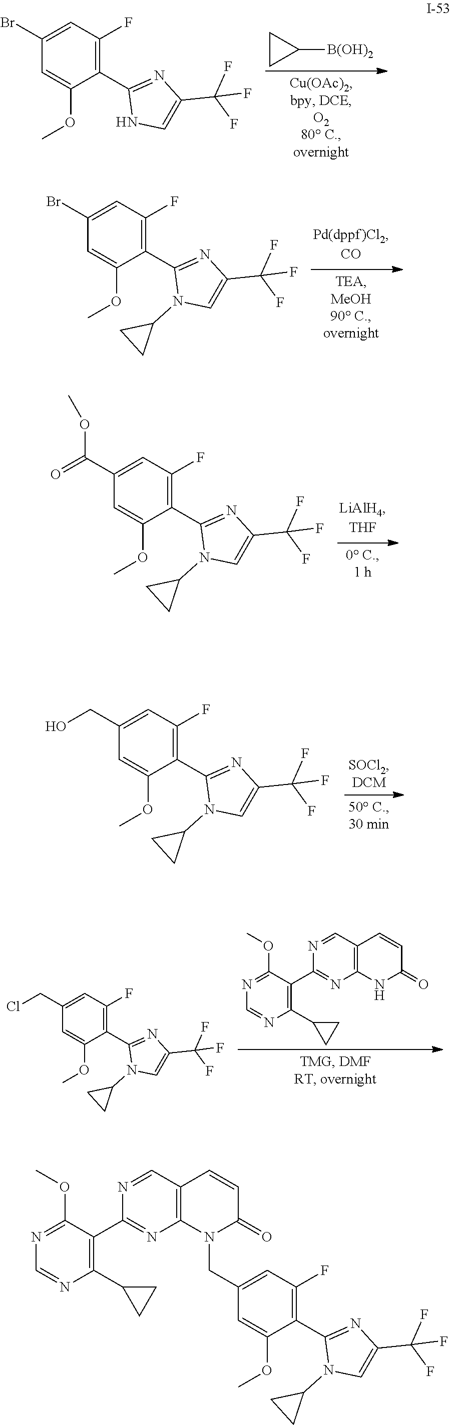

### Step 1: 2-(4-Bromo-2-Fluoro-6-Methoxyphenyl)-1-Cyclopropyl-4-(Trifluoromethyl)Imidazole

To a stirred mixture of 2-(4-bromo-2-fluoro-6-methoxyphenyl)-4-(trifluoromethyl)-1H-imidazole (200 mg, 0.590 mmol, 1 equiv) and cyclopropylboronic acid (101.33 mg, 1.180 mmol, 2 equiv) in DCE (4 mL, 20 equiv) were added bipyridyl (276.37 mg, 1.770 mmol, 3 equiv) and Cu(OAc)₂ (321.39 mg, 1.770 mmol, 3 equiv) at room temperature under hydrogen atmosphere. The resulting mixture was stirred for overnight at 80° C. under oxygen gas atmosphere. The resulting mixture was filtered, the filter cake was washed with DCM (3×5 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford 2-(4-bromo-2-fluoro-6-methoxyphenyl)-1-cyclopropyl-4-(trifluoromethyl)imidazole (30 mg, 13.41%) as a black solid. LCMS (ESI) m/z 381.0 [M+H]

### Step 2: Methyl 4-[1-Cyclopropyl-4-(Trifluoromethyl)Imidazo]-2-Yl]-3-Fluoro-5-Methoxybenzoate

To a stirred 2-(4-bromo-2-fluoro-6-methoxyphenyl)-1-cyclopropyl-4-(trifluoromethyl)imidazole (135 mg, 0.356 mmol, 1 equiv) in MeOH (2.7 mL, 66.687 mmol, 187.29 equiv) were added Pd(dppf)Cl₂ (52.11 mg, 0.071 mmol, 0.2 equiv) and TEA (108.09 mg, 1.068 mmol, 3 equiv) at room temperature under carbon monoxide atmosphere. The resulting mixture was stirred for overnight at 90° C. under carbon monoxide atmosphere. The resulting mixture was filtered; the filter cake was washed with MeOH (3×1 mL). The filtrate was concentrated under reduced pressure. The residue was purified by silica gel column chromatography, (eluting with PE/EA (5:1)) to afford methyl 4-[1-cyclopropyl-4-(trifluoromethyl)imidazo]-2-yl]-3-fluoro-5-methoxybenzoate (60 mg, 47.03%) as a colorless oil. LC-MS (ESI) m/z 359.1 [M+H].

### Step 3: {4-[1-Cyclopropyl-4-(Trifluoromethyl)Imidazo]-2-Yl]-3-Fluoro-5-Methoxyphenyl}Methanol

To a stirred methyl 4-[1-cyclopropyl-4-(trifluoromethyl)imidazo]-2-yl]-3-fluoro-5-methoxybenzoate (120 mg, 0.335 mmol, 1 equiv) in THF (2.4 mL) was added LiAlH₄ (38.13 mg, 1.005 mmol, 3.0 equiv) in portions at 0° C. under nitrogen atmosphere. The resulting mixture was stirred for 1 h at 0° C. under nitrogen atmosphere. The reaction was quenched with 10H2O.Na₂SO₄ at 0° C. The resulting mixture was filtered, the filter cake was washed with THF (3×2 mL). The filtrate was concentrated under reduced pressure. The resulting mixture was extracted with EtOAc (3×5 mL). The combined organic layers were washed with brine (2×2 mL), dried over anhydrous Na₂SO₄, filtered and concentrated under reduced pressure. The residue was purified by silica gel column chromatography, (eluting with PE/EA (1:1)) to afford {4-[1-cyclopropyl-4-(trifluoromethyl)imidazol-2-yl]-3-fluoro-5-methoxyphenyl}methanol (72 mg, 65.09%) as a colorless oil. LC-MS (ESI) m/z 331.2 [M+H]

### Step 4: 2-[4-(Chloromethyl)-2-Fluoro-6-Methoxyphenyl]-1-Cyclopropyl-4-(Trifluoromethyl)Imidazole

To a stirred {4-[1-cyclopropyl-4-(trifluoromethyl)imidazo]-2-yl]-3-fluoro-5-methoxyphenyl}methanol (70 mg, 0.212 mmol, 1 equiv) in DCM (1.4 mL, 20 equiv) was added SOCl₂ (75.64 mg, 0.636 mmol, 3 equiv) at room temperature under nitrogen atmosphere. The resulting mixture was stirred for 30 min at 50° C. under nitrogen atmosphere. The reaction was quenched with Water at 0° C. The mixture neutralized to pH 7 with saturated NaHCO₃(aq.). The resulting mixture was extracted with CH₂Cl₂ (3×2 mL). The combined organic layers were washed with brine (2×2 mL), dried over anhydrous Na₂SO₄, filtered and concentrated under reduced pressure to afford 2-[4-(chloromethyl)-2-fluoro-6-methoxyphenyl]-1-cyclopropyl-4-(trifluoromethyl)imidazole (75 mg, 101.48%) as a light yellow oil. LC-MS (ESI) m/z 349.1 [M+H].

### Step 5: 8-({4-[1-Cyclopropyl-4-(Trifluoromethyl)Imidazol-2-Yl]-3-Fluoro-5-Methoxyphenyl}Methyl)-2-(4-Cyclopropyl-6-Methoxypyrimidin-5-Yl)Pyrido[2,3-d]Pyrimidin-7-One

To a stirred solution of 2-[4-(chloromethyl)-2-fluoro-6-methoxyphenyl]-1-cyclopropyl-4-(trifluoromethyl)imidazole (79.71 mg, 0.229 mmol, 0.9 equiv) and 2-(4-cyclopropyl-6-methoxypyrimidin-5-yl)-8H-pyrido[2,3-d]pyrimidin-7-one (75 mg, 0.254 mmol, 1.00 equiv) in DMF (1.5 mL, 20 equiv) were added 1,1,3,3-tetramethylguanidine (43.88 mg, 0.381 mmol, 1.5 equiv) at room temperature under nitrogen atmosphere. The resulting mixture was stirred for overnight at room temperature under nitrogen atmosphere. The resulting mixture was filtered, the filter cake was washed with DMF (3×1 mL). The crude product (50 mg) was purified by Prep-HPLC with the following conditions (Column: Xselect CSH F-Phenyl OBD column, mm, 51 μm; Mobile Phase A: Water(10 mmol/L NH₄HCO₃), Mobile Phase B: ACN; Flow rate: 50 mL/min; Gradient: 35% B to 55% B in 8 min, 55% B; Wave Length: 254/220 nm; RT 1(min): 7.80; Number Of Runs: 0) to afford 8-({4-[1-cyclopropyl-4-(trifluoromethyl)imidazol-2-yl]-3-fluoro-5-methoxyphenyl}methyl)-2-(4-cyclopropyl-6-methoxypyrimidin-5-yl)pyrido[2,3-d]pyrimidin-7-one (42.9 mg, 27.77%) as a white solid. ¹H NMR (400 MHz, DMSO-d6) δ 9.30 (s, 1H), 8.69 (s, 1H), 8.17 (d, J=9.6 Hz, 1H), 7.91 (d, J=1.3 Hz, 1H), 7.05 (d, J=5.6 Hz, 1H), 6.92 (d, J=9.5 Hz, 1H), 6.72 (dd, J=9.9, 1.4 Hz, 1H), 5.56 (d, J=6.6 Hz, 2H), 3.82 (s, 3H), 3.71 (d, J=1.6 Hz, 3H), 3.18 (ddd, J=10.6, 7.1, 4.3 Hz, 1H), 1.76 (td, J=8.1, 4.1 Hz, 1H), 1.02 (s, 2H), 0.92 (s, 1H), 0.80 (dd, J=8.0, 3.1 Hz, 2H), 0.69 (s, 2H). LC-MS (ESI) m/z 608.3 [M+H]

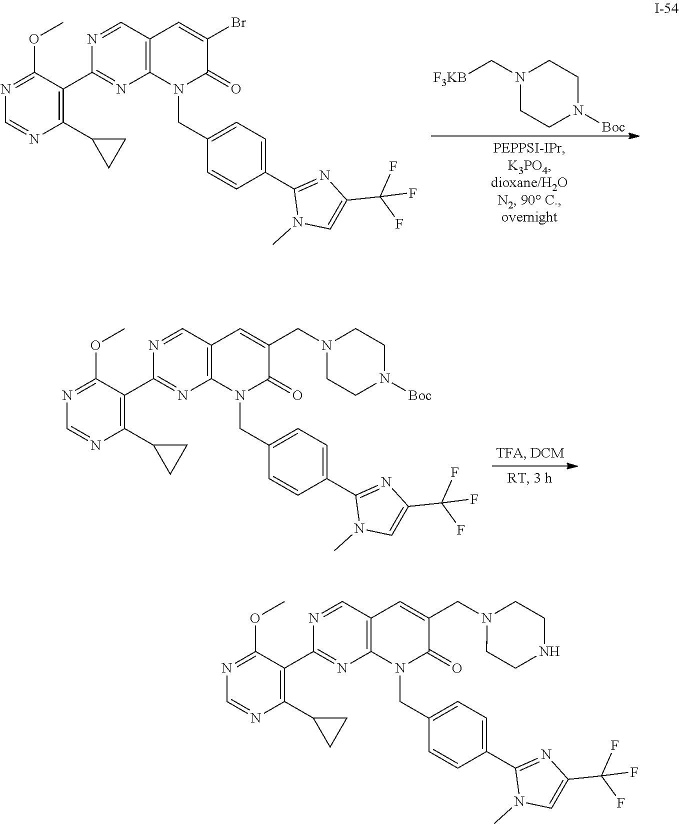

### Step 1: Tert-Butyl 4-{12-(4-Cyclopropyl-6-Methoxypyrimidin-5-Yl)-8-({4-[1-Methyl-4-(Trifluoromethyl)Imidazol-2-Yl]Phenyl}Methyl)-7-Oxopyrido[2,3-d]Pyrimidin-6-Yl]Methyl}Piperazine-1-Carboxylate

To a stirred solution of 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (130 mg, 0.212 mmol, 1 equiv) and tert-butyl 4-{[trifluoro(potassio)-lambda5-boranyl]methyl}piperazine-1-carboxylate (129.99 mg, 0.424 mmol, 2 equiv) in dioxane (2 mL) and H₂O (0.4 mL) were added K₃PO₄ (90.12 mg, 0.424 mmol, 2 equiv) and PEPPSI-IPr (28.91 mg, 0.042 mmol, 0.2 equiv) at room temperature. The resulting mixture was stirred for overnight at 90° C. under nitrogen atmosphere. The resulting mixture was filtered, the filter cake was washed with THF (2×3 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford tert-butyl 4-{[2-(4-cyclopropyl-6-methoxypyrimidin-5-yl)-8-({4-[1-methyl-4-(trifluoromethyl)imidazo]-2-yl]phenyl}methyl)-7-oxopyrido[2,3-d]pyrimidin-6-yl]methyl}piperazine-1-carboxylate (145 mg, 93.34%) as an off-white solid. LC-MS (ESI) m/z 732.7 [M+H]

### Step 2: 2-(4-cyclopropyl-6-methoxypyrimidin-5-yl)-8-({4-[1-methyl-4-(trifluoromethyl)imidazol-2-yl]phenyl}methyl)-6-(piperazin-1-ylmethyl)pyrido[2,3-d]pyrimidin-7-one

To a stirred solution of tert-butyl 44-[2-(4-cyclopropyl-6-methoxypyrimidin-5-yl)-8-({4-[1-methyl-4-(trifluoromethyl)imidazo]-2-yl]phenyl methyl)-7-oxopyrido[2,3-d]pyrimidin-6-yl]methyl}piperazine-1-carboxylate (30 mg, 0.041 mmol, 1 equiv) in DCM (1 mL) was added TFA (0.3 mL, 4.039 mmol, 98.52 equiv) at room temperature. The resulting mixture was stirred for 3 h at room temperature. The resulting mixture was concentrated under reduced pressure. The residue was purified by Prep-HPLC with the following conditions (Column: Xselect CSH C18 OBD Column 30*150 mm 51 μm, n; Mobile Phase A: Water(0.1% FA), Mobile Phase B: ACN; Flow rate: 60 mL/min; Gradient: 15% B to 35% B in 7 min, 35% B; Wave Length: 254/220 nm; RT1(min): 6.82) to afford 2-(4-cyclopropyl-6-methoxypyrimidin-5-yl)-8-({4-[1-methyl-4-(trifluoromethyl)imidazo]-2-yl]phenyl methyl)-6-(piperazin-1-ylmethyl)pyrido[2,3-d]pyrimidin-7-one (12.5 mg, 47.11%) as an off-white solid. ¹H NMR (400 MHz, DMSO-d6) δ 9.33 (s, 1H), 8.69 (s, 1H), 8.34 (s, 1H), 8.12 (s, 1H), 7.91 (d, J=1.4 Hz, 1H), 7.69-7.55 (m, 2H), 7.40 (d, J=8.1 Hz, 2H), 5.59 (s, 2H), 3.83 (s, 3H), 3.74 (s, 3H), 3.49 (s, 2H), 2.90 (d, J=5.5 Hz, 4H), 2.53 (s, 4H), 1.72 (tq, J=8.1, 4.6, 4.0 Hz, 1H), 1.02 (p, J=3.5 Hz, 2H), 0.77 (dq, J=7.0, 3.4 Hz, 2H). LC-MS (ESI) m/z 632.25 [M+H]

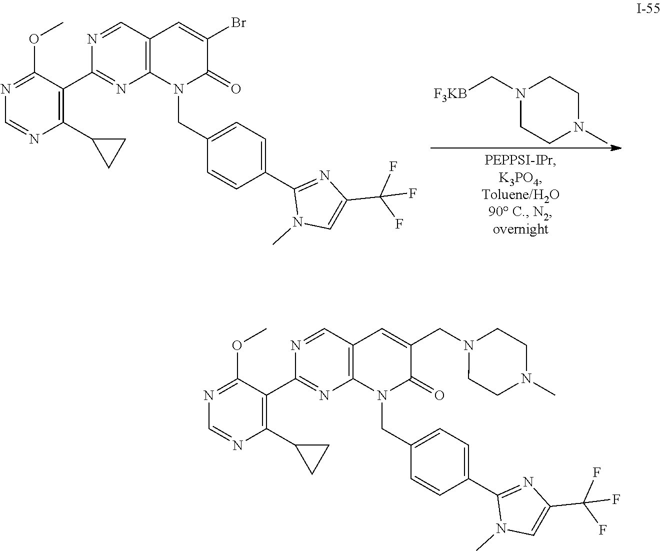

To a stirred solution of 6-bromo-2-(4-cyclopropyl-6-methoxypyrimidin-5-yl)-8-({4-[1-methyl-4-(trifluoromethyl)imidazo]-2-yl]phenyl methyl)pyrido[2,3-d]pyrimidin-7-one (30 mg, 0.049 mmol, 1 equiv) and 1-methyl-4-{[trifluoro(potassio)-lambda5-boranyl]methyl}piperazine (21.56 mg, 0.098 mmol, 2 equiv) in dioxane (1 mL) and H₂O (0.2 mL) were added K₃PO₄ (20.80 mg, 0.098 mmol, 2 equiv) and PEPPSI-IPr (6.67 mg, 0.010 mmol, 0.2 equiv) at room temperature. The resulting mixture was stirred for overnight at 90° C. under nitrogen atmosphere. The resulting mixture was concentrated under reduced pressure. The residue was purified by Prep-HPLC with the following conditions (Column: YMC-Actus Triart C18 ExRS, mm, 51 μm; Mobile Phase A: Water(10 mmol/L NH₄HCO₃), Mobile Phase B: ACN; Flow rate: 60 mL/min; Gradient: 32% B to 52% B in 7 min, 52% B; Wave Length: 254/220 nm; RT1(min): 6.70) to afford 2-(4-cyclopropyl-6-methoxypyrimidin-5-yl)-8-({4-[1-methyl-4-(trifluoromethyl)imidazo]-2-yl]phenyl methyl)-6-[(4-methylpiperazin-1-yl)methyl]pyrido[2,3-d]pyrimidin-7-one (8.5 mg, 26.55%) as an off-white solid. ¹H NMR: (400 MHz, DMSO-d6) δ 9.34 (s, 1H), 8.69 (s, 1H), 8.08 (s, 1H), 7.91 (s, 1H), 7.62 (d, J=8.0 Hz, 2H), 7.40 (d, J=8.1 Hz, 2H), (s, 2H), 3.83 (s, 3H), 3.74 (s, 3H), 3.49 (s, 2H), 2.51 (s, 2H), 2.50 (s, 2H), 2.45-2.31 (m, 4H), 2.19 (s, 3H), 1.73 (tq, J=8.2, 4.7, 3.5 Hz, 1H), 1.25-1.18 (m, 1H), 1.01 (p, J=3.6 Hz, 2H), (dq, J=7.1, 3.5 Hz, 2H). LC-MS (ESI) m/z 646.25 [M+H]

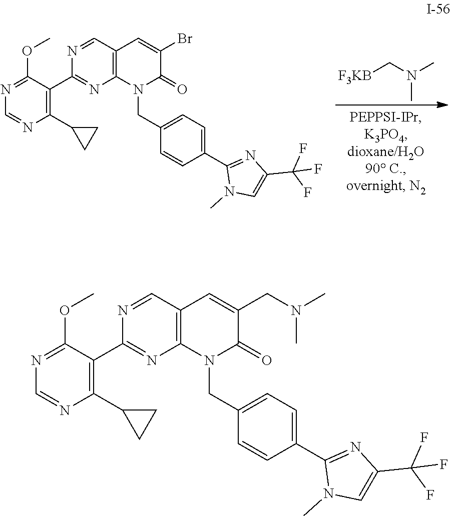

To a stirred solution of 6-bromo-2-(4-cyclopropyl-6-methoxypyrimidin-5-yl)-8-({4-[1-methyl-4-(trifluoromethyl)imidazo]-2-yl]phenyl methyl)pyrido[2,3-d]pyrimidin-7-one (30 mg, 0.049 mmol, 1 equiv) and dimethyl({[trifluoro(potassio)-lambda5-boranyl]methyl})amine (16.17 mg, 0.098 mmol, 2 equiv) in dioxane (1 mL) and H₂O (0.2 mL) were added K₃PO₄ (20.80 mg, 0.098 mmol, 2 equiv) and PEPPSI-IPr (6.67 mg, 0.010 mmol, 0.2 equiv) at room temperature. The resulting mixture was stirred for overnight at 90° C. under nitrogen atmosphere. The resulting mixture was concentrated under reduced pressure. The residue was purified by Prep-HPLC with the following conditions (Column: Sunfire prep C18 column, 30*150 mm, 51 μm; Mobile Phase A: Water(0.1% FA), Mobile Phase B: ACN; Flow rate: 60 mL/min; Gradient: 15% B to 30% B in 7 min, 30% B; Wave Length: 254/220 nm; RT1(min): 6.58) to afford 2-(4-cyclopropyl-6-methoxypyrimidin-5-yl)-6-[(dimethylamino)methyl]-8-({4-[1-methyl-4-(trifluoromethyl)imidazo]-2-yl]phenyl}methyl)pyrido[2,3-d]pyrimidin-7-one (8.1 mg, 27.74%) as an off-white solid. ¹H NMR (400 MHz, DMSO-d6) δ 9.33 (s, 1H), 8.69 (s, 1H), 8.11 (s, 1H), 7.93-7.88 (m, 1H), 7.63 (d, J=8.2 Hz, 2H), 7.41 (d, J=8.2 Hz, 2H), 5.59 (s, 2H), 3.83 (s, 3H), 3.74 (s, 3H), 3.43 (s, 2H), 2.28 (s, 6H), 1.73 (tt, J=8.2, 4.5 Hz, 1H), 1.02 (m, J=3.5 Hz, 2H), 0.77 (dq, J=7.1, 3.5 Hz, 2H). LC-MS (ESI) m/z 591.30 [M+H]

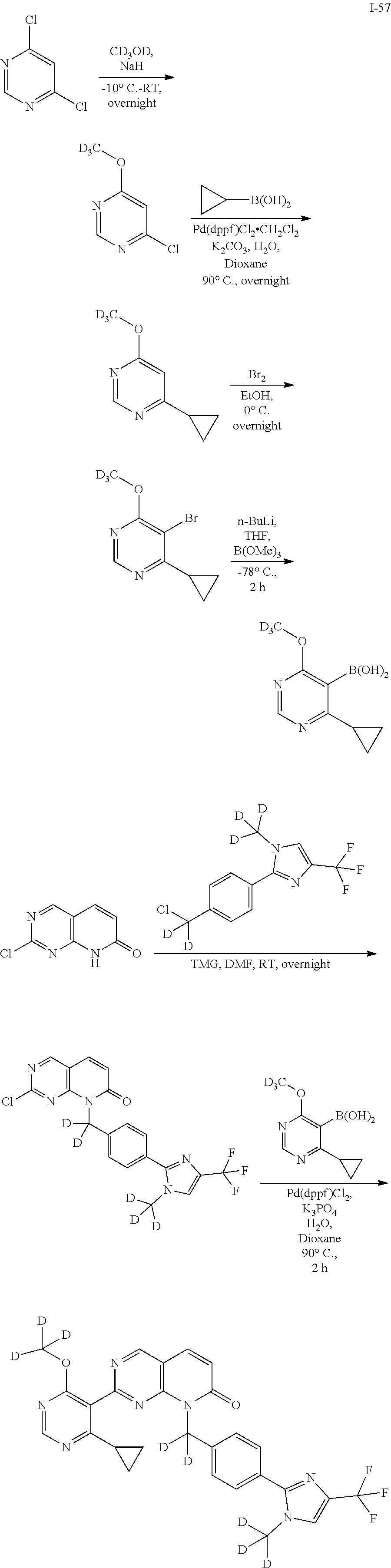

### Step 1: 4-Chloro-6-(2H3)Methoxypyrimidine

To a stirred solution of 4,6-dichloropyrimidine (1 g, 6.713 mmol, 1 equiv) in CD 3 OD (5 mL, 112.294 mmol, 16.73 equiv) was added NaH (0.28 g, 11.668 mmol, 1.74 equiv) dropwise at −10° C. under nitrogen atmosphere. The resulting mixture was stirred for overnight at room temperature under nitrogen atmosphere. The reaction was quenched by the addition of water (20 mL) at room temperature. The resulting mixture was extracted with EtOAc (3×100 mL). The combined organic layers were washed with brine (2×100 mL), dried over anhydrous Na₂SO₄, filtered and concentrated under reduced pressure. The residue was purified by silica gel column chromatography, (eluting with PE/EA (1:1)) to afford 4-chloro-6-(2H3)methoxypyrimidine (1.2 g, 90.3%) as a colorless oil. LC-MS (ESI) m/z 147 [M+H].

### Step 2: 4-Cyclopropyl-6-(2H3)Methoxypyrimidine

A solution of 4-chloro-6-(2H3)methoxypyrimidine (50 mg, 0.339 mmol, 1 equiv), cyclopropylboronic acid (87.31 mg, 1.017 mmol, 3 equiv), Pd(dppf)Cl₂.CH₂Cl₂ (41.40 mg, 0.051 mmol, 0.15 equiv), K₂CO₃ (93.65 mg, 0.678 mmol, 2 equiv) and H₂O (0.5 mL, 27.755 mmol, 81.92 equiv) in Dioxane (2 mL, 23.608 mmol, 69.68 equiv) was stirred for overnight at under nitrogen atmosphere. The mixture was allowed to cool down to room temperature. The resulting mixture was filtered, the filter cake was washed with EtOAc (3×10 mL). The filtrate was concentrated under reduced pressure. The residue was purified by reverse flash chromatography (C18 silica gel column, eluting with 0% to 100% MeCN/water (0.1% FA) in 10 min with UV detector (254 nm) to afford 4-cyclopropyl-6-(2H3)methoxypyrimidine (25 mg, 48.17%) as a yellow oil. LC-MS (ESI) m/z 154 [M+H].

### Step 3: 5-Bromo-4-Cyclopropyl-6-(2H3)Methoxypyrimidine

To a stirred solution of 4-cyclopropyl-6-(2H3)methoxypyrimidine (3.8 g, 24.804 mmol, 1 equiv) in EtOH (30 mL, 516.399 mmol, 20.82 equiv) was added Brom (11.89 g, 74.412 mmol, 3 equiv) dropwise at 0° C. under nitrogen atmosphere. The mixture was stirred for overnight at room temperature. The precipitated solids were collected by filtration and washed with EtOH (3×100 mL). The residue was dissolved in water (50 mL). The mixture was basified to pH 7 with saturated NaHCO₃(aq.). The resulting mixture was extracted with EtOAc (3×100 mL). The combined organic layers were washed with brine (2×100 mL), dried over anhydrous Na₂SO₄, filtered and concentrated under reduced pressure to afford 5-bromo-4-cyclopropyl-6-(2H3)methoxypyrimidine (2.2 g, 38.21%) as an off-white solid. LC-MS (ESI) m/z 232 [M+H].

### Step 4: 4-Cyclopropyl-6-(2113)Methoxypyrimidin-5-Ylboronic Acid

A solution of 5-bromo-4-cyclopropyl-6-(2H3)methoxypyrimidine (2 g, 8.617 mmol, 1 equiv) in THF (20 mL, 246.855 mmol, 28.65 equiv) was treated with B(OMe) 3 (1.34 g, 12.926 mmol, 1.5 equiv) for 5 min at room temperature under nitrogen atmosphere followed by the addition of n-BuLi (0.83 g, 12.926 mmol, 1.5 equiv) dropwise at −78° C. The resulting mixture was stirred for 2 h at room temperature under nitrogen atmosphere. The reaction was quenched with 1M HCl at room temperature. The mixture was neutralized to pH 7 with saturated NaHCO₃(aq.). The resulting mixture was extracted with EtOAc (3×100 mL). The combined organic layers were washed with water (2×50 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm) to afford 4-cyclopropyl-6-(2H3)methoxypyrimidin-5-ylboronic acid (770 mg, 45.35%) as an off-white solid. LC-MS (ESI) m/z 198 [M+H].

### Step 5: 2-Chloro-8-({4-[1-(2H3)Methyl-4-(Trifluoromethyl)Imidazol-2-Yl]Phenyl}(2112)Methyl)Pyrido[2,3-d]Pyrimidin-7-One

A solution of 2-chloro-8H-pyrido[2,3-d]pyrimidin-7-one (100 mg, 0.551 mmol, 1 equiv), N,N,N′,N′-tetramethylguanidine (95.15 mg, 0.827 mmol, 1.5 equiv) and 2-{4-[chloro(2H2)methyl]phenyl}-1-(2H3)methyl-4-(trifluoromethyl)imidazole (154.04 mg, 0.551 mmol, 1 equiv) in dimethylformamide (5 mL) was stirred for 2 h at room temperature under nitrogen atmosphere. The residue was purified by reverse flash chromatography (C18 silica gel column, eluting with 0% to 100% MeCN/water (0.1% FA) in 10 min with UV detector (254 nm) to afford 2-chloro-8-({4-[1-(2H3)methyl-4-(trifluoromethyl)imidazol-2-yl]phenyl}(2H2)methyl)pyrido[2,3-d]pyrimidin-7-one (89 mg, 38.04%) as an off-white solid. LC-MS (ESI) m/z 425 [M+H].

### Step 6: 2-[4-Cyclopropyl-6-(2H3)Methoxypyrimidin-5-Yl]-8-({4-[1-(2113)Methyl-4-(Trifluoromethyl)Imidazol-2-Yl]Phenyl}(2H2)Methyl)Pyrido[2,3-d]Pyrimidin-7-One

A solution of 2-chloro-8-({4-[1-(2H3)methyl-4-(trifluoromethyl)imidazol-2-yl]phenyl}(2H2)methyl)pyrido[2,3-d]pyrimidin-7-one (50 mg, 0.118 mmol, 1 equiv), 4-cyclopropyl-6-(2H3)methoxypyrimidin-5-ylboronic acid (27.83 mg, 0.142 mmol, 1.2 equiv), Pd(dppf)Cl₂ (12.92 mg, 0.018 mmol, 0.15 equiv), K₃PO₄ (49.97 mg, 0.236 mmol, 2 equiv) and H₂O (1 mL, 55.509 mmol, 471.63 equiv) in Dioxane (4 mL, 47.216 mmol, 401.16 equiv) was stirred for 2 h at 90° C. under nitrogen atmosphere. The mixture was allowed to cool down to room temperature. The residue was purified by reverse flash chromatography (C18 silica gel column, eluting with 0% to 100% MeCN/water (0.1% FA) in 10 min with UV detector (254 nm) to afford 2-[4-cyclopropyl-6-(2H3)methoxypyrimidin-5-yl]-8-({4-[1-(2H3)methyl-4-(trifluoromethyl)imidazol-2-yl]phenyl}(2H2)methyl)pyrido[2,3-d]pyrimidin-7-one (9 mg, 13.82%) as an off-white solid. ¹H NMR (300 MHz, DMSO-d6) δ 9.30 (s, 1H), 8.69 (s, 1H), 8.17 (d, J=9.6 Hz, 1H), 7.92 (q, J=1.3 Hz, 1H), 7.67-7.60 (m, 2H), 7.45-7.38 (m, 2H), 6.91 (d, J=9.5 Hz, 1H), 1.72 (dq, J=8.2, 4.5, 4.1 Hz, 1H), 1.03 (q, J=3.9 Hz, 2H), 0.77 (dd, J=8.0, 3.3 Hz, 2H). LC-MS (ESI) m/z 542 [M+H].

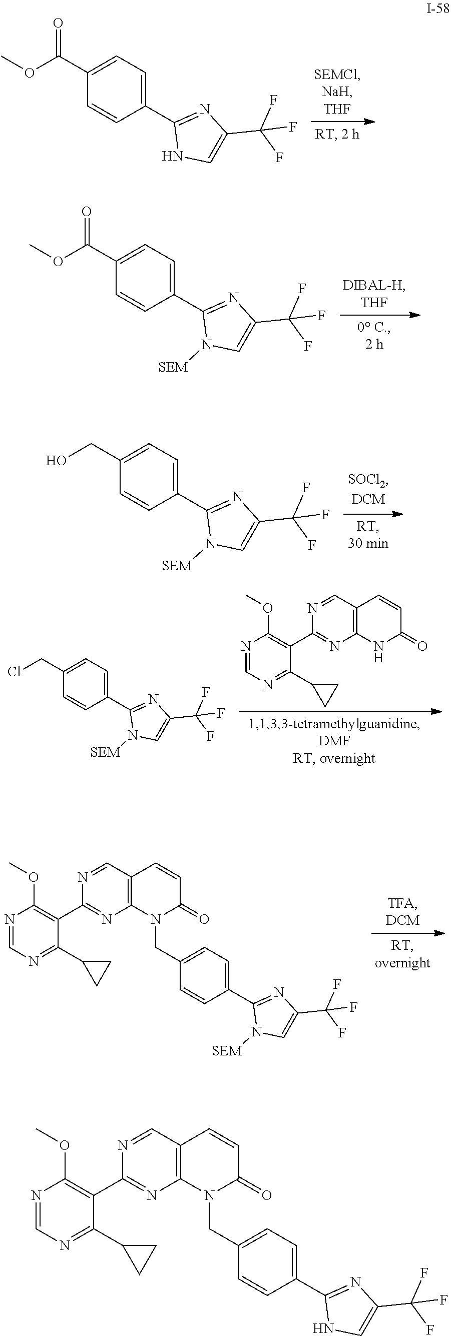

### Step 1: Methyl 4-[4-(Trifluoromethyl)-1-{[2-(Trimethylsilyl)Ethoxy]Methyl}Imidazol-2-Yl]Benzoate

A solution of methyl 4-[4-(trifluoromethyl)-1H-imidazol-2-yl]benzoate (2 g, 7.402 mmol, 1 equiv) in THF (40 mL, 493.711 mmol, 66.70 equiv) was treated with NaH (355.25 mg, 14.804 mmol, 2 equiv) for 30 min at 0° C. under nitrogen atmosphere followed by the addition of SEMCl (1.851 g, 11.103 mmol, 1.5 equiv) dropwise at 0° C. The resulting mixture was stirred for 2 h at room temperature under nitrogen atmosphere. The reaction was quenched with Water/Ice at 0° C. The resulting mixture was extracted with EtOAc (3×100 mL). The combined organic layers were washed with brine (3×100 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford methyl 4-[4-(trifluoromethyl)-1-{[2-(trimethylsilyl)ethoxy]methyl}imidazol-2-yl]benzoate (2.1 g, 70.85%) as a yellow oil. LC-MS (ESI) m/z 401.1 [M+H]

### Step 2: 4-[4-(Trifluoromethyl)-1-{[2-(Trimethylsilyl)Ethoxyl Methyl}Imidazol-2-Yl]Phenyl}Methanol

To a stirred mixture of methyl 4-[4-(trifluoromethyl)-1-{[2-(trimethylsilyl)ethoxy]methyl}imidazol-2-yl]benzoate (3 g, 7.491 mmol, 1 equiv) in THF (60 mL, 740.566 mmol, 98.86 equiv) was added DIBAL-H (29.96 mL, 29.964 mmol, 4 equiv) dropwise at 0° C. under nitrogen atmosphere. The resulting mixture was stirred for 2h at under nitrogen atmosphere. The reaction was quenched with Water/Ice at 0° C. The resulting mixture was extracted with EtOAc (3×100 mL). The combined organic layers were washed with brine (3×100 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford {4-[4-(trifluoromethyl)-1-{[2-(trimethyl silyl)ethoxy]methyl imidazol-2-yl]phenyl}methanol (2.74 g, 98.20%) as a yellow oil. LC-MS (ESI) m/z 373.1 [M+H]

### Step 3: 2-[4-(Chloromethyl)Phenyl]-4-(Trifluoromethyl)-1-{12-(Trimethylsilyl)Ethoxy]Methyl}Imidazole

To a stirred mixture of {4-[4-(trifluoromethyl)-1-{[2-(trimethyl silyl)ethoxy]methyl imidazol-2-yl]phenyl}methanol (1 g, 2.685 mmol, 1 equiv) in DCM (20 mL, 314.612 mmol, 117.18 equiv) was added thionyl chloride (0.80 g, 6.713 mmol, 2.5 equiv) dropwise at 0° C. under nitrogen atmosphere. The resulting mixture was stirred for 30 min at room temperature under nitrogen atmosphere. The reaction was quenched with Water/Ice at 0° C. The resulting mixture was extracted with EtOAc (3×100 mL). The combined organic layers were washed with brine (3×100 mL), dried over anhydrous Na₂SO₄, filtered and concentrated under reduced pressure. The residue was purified by silica gel column chromatography, (eluting with PE/EA (9:1)) to afford 2-[4-(chloromethyl)phenyl]-4-(trifluoromethyl)-1-{[2-(trimethylsilyl)ethoxy]methyl imidazole (870 mg, 82.89%) as a yellow oil. LC-MS (ESI) m/z 391.1 [M+H]

### Step 4: 2-(4-Cyclopropyl-6-Methoxypyrimidin-5-Yl)-8-({4-[4-(Trifluoromethyl)-1-{12-(Trimethylsilyl)Ethoxy]Methyl}Imidazol-2-Yl]Phenyl}Methyl)Pyrido[2,3-d]Pyrimidin-7-One

To a stirred mixture of 2-[4-(chloromethyl)phenyl]-4-(trifluoromethyl)-1-{[2-(trimethylsilyl)ethoxy]methyl}imidazole (661.88 mg, 1.693 mmol, 1 equiv) and 2-(4-cyclopropyl-6-methoxypyrimidin-5-yl)-8H-pyrido[2,3-d]pyrimidin-7-one (500 mg, 1.693 mmol, 1.00 equiv) in DMF (10 mL, 129.215 mmol, 76.32 equiv) was added 1,1,3,3-tetramethylguanidine (292.52 mg, 2.540 mmol, 1.5 equiv) dropwise at room temperature under nitrogen atmosphere. The resulting mixture was stirred for overnight at room temperature under nitrogen atmosphere. The reaction was quenched with water. The resulting mixture was extracted with EtOAc (3×50 mL). The combined organic layers were washed with brine (3×50 mL), dried over anhydrous Na₂SO₄, filtered and concentrated under reduced pressure. The residue was purified by silica gel column chromatography, (eluting with PE/EA (1:1)) to afford 2-(4-cyclopropyl-6-methoxypyrimidin-5-yl)-8-({4-[4-(trifluoromethyl)-1-{[2-(trimethyl silyl)ethoxy]methyl imidazol-2-yl]phenyl methyl)pyri do[2,3-d]pyrimidin-7-one (1 g, as a colorless oil. LC-MS (ESI) m/z 650.2 [M+H]

### Step 5: 2-(4-Cyclopropyl-6-Methoxypyrimidin-5-Yl)-8-({4-[4-(Trifluoromethyl)-1H-Imidazol-2-Yl]Phenyl}Methyl)Pyrido[2,3-d]Pyrimidin-7-One

A mixture of 2-(4-cyclopropyl-6-methoxypyrimidin-5-yl)-8-({4-[4-(trifluoromethyl)-1-{[2-(trimethylsilyl)ethoxy]methyl imidazol-2-yl]phenyl methyl)pyrido[2,3-d]pyrimidin-7-one (500 mg, 0.770 mmol, 1 equiv) in TFA(5 mL) and DCM (5 mL, 78.653 mmol, 102.21 equiv) was stirred for overnight at room temperature under nitrogen atmosphere. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[4-(trifluoromethyl)-1H-imidazol-2-yl]phenyl methyl)pyri do[2,3-d]pyrimidin-7-one (300 mg, 73.62%) as an off-white solid. ¹H NMR (400 MHz, DMSO-d6) δ 13.15 (s, 1H), 9.28 (d, J=Hz, 1H), 8.69 (s, 1H), 8.15 (dd, J=9.6, 0.8 Hz, 1H), 7.95-7.83 (m, 3H), 7.39 (d, J=8.1 Hz, 2H), 6.90 (d, J=9.5 Hz, 1H), 5.54 (s, 2H), 3.83 (d, J=0.9 Hz, 3H), 2.08 (d, J=0.9 Hz, 2H), 1.72 (tt, J=8.2, 4.6 Hz, 1H), 1.02 (p, J=3.7 Hz, 2H), 0.76 (dq, J=7.1, 3.6 Hz, 2H). LC-MS (ESI) m/z 520.2 [M+H]

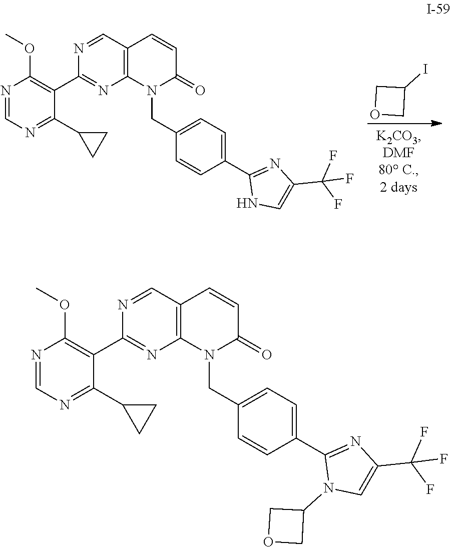 [SMILES: COc1ncnc(C2CC2)c1-c1ncc2ccc(=O)n(Cc3ccc(-c4nc(C(F)(F)F)c[nH]4)cc3)c2n1 ; IC1COC1 ; COc1ncnc(C2CC2)c1-c1ncc2ccc(=O)n(Cc3ccc(-c4nc(C(F)(F)F)cn4C4COC4)cc3)c2n1]

To a stirred mixture of 2-(4-cyclopropyl-6-methoxypyrimidin-5-yl)-8-({4-[4-(trifluoromethyl)-1H-imidazol-2-yl]phenyl}methyl)pyrido[2,3-d]pyrimidin-7-one (20 mg, 0.038 mmol, 1 equiv) and K₂CO₃ (21.28 mg, 0.152 mmol, 4 equiv) in DMF (0.5 mL, 6.461 mmol, 167.81 equiv) was added 3-iodooxetane (28.33 mg, 0.152 mmol, 4 equiv) dropwise at room temperature under nitrogen atmosphere. The resulting mixture was stirred for 2 days at under nitrogen atmosphere. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[1-(oxetan-3-yl)-4-(trifluoromethyl)imidazol-2-yl]phenyl}methyl)pyrido[2,3-d]pyrimidin-7-one (16 mg, 71.56%) as an off-white solid. ¹H NMR (400 MHz, DMSO-d6) δ 9.29 (s, 1H), 8.69 (s, 1H), 8.53-8.43 (m, 1H), 8.16 (d, J=9.5 Hz, 1H), 7.42 (q, J=8.3 Hz, 4H), 6.91 (d, J=9.5 Hz, 1H), 5.57 (s, 2H), 5.46 (p, J=7.0 Hz, 1H), 4.81 (dt, J=21.9, 7.0 Hz, 4H), 3.83 (s, 3H), 1.72 (dd, J=8.5, 4.4 Hz, 1H), 1.02 (t, J=3.9 Hz, 2H), 0.77 (dd, J=7.9, 3.3 Hz, 2H). LC-MS (ESI) m/z 576.2 [M+H]

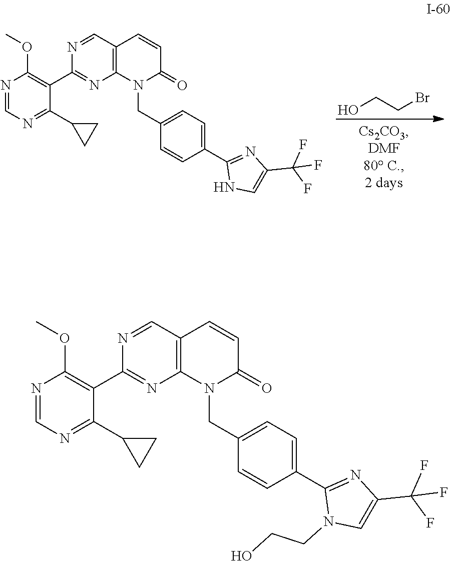

To a stirred mixture of 2-(4-cyclopropyl-6-methoxypyrimidin-5-yl)-8-({4-[4-(trifluoromethyl)-1H-imidazol-2-yl]phenyl}methyl)pyrido[2,3-d]pyrimidin-7-one (20 mg, 0.038 mmol, 1 equiv) and Cs₂CO₃ (37.63 mg, 0.114 mmol, 3 equiv) in DMF (0.5 mL, 6.461 mmol, 167.81 equiv) was added 2-bromoethanol (19.24 mg, 0.152 mmol, 4 equiv) dropwise at room temperature under nitrogen atmosphere. The resulting mixture was stirred for 2 days at under nitrogen atmosphere. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[1-(2-hy droxy ethyl)-4-(trifluoromethyl)imidazol-2-yl]phenyl}methyl)pyrido[2,3-d]pyrimidin-7-one (5.1 mg, 23.37%) as an off-white solid. ¹H NMR (400 MHz, DMSO-d6) δ 9.29 (s, 1H), 8.69 (s, 1H), 8.16 (d, J=9.6 Hz, 1H), 7.95 (d, J=1.4 Hz, 1H), 7.61 (d, J=8.1 Hz, 2H), 7.40 (d, J=8.1 Hz, 2H), 6.90 (d, J=9.6 Hz, 1H), 5.57 (s, 2H), 5.07 (s, 1H), 4.05 (t, J=5.4 Hz, 2H), 3.82 (s, 3H), 3.68 (d, J=5.2 Hz, 2H), 1.79-1.66 (m, 1H), 1.01 (s, 2H), 0.77 (dd, J=7.9, 3.4 Hz, 2H). LC-MS-(ESI) m/z 564.2 [M+H]

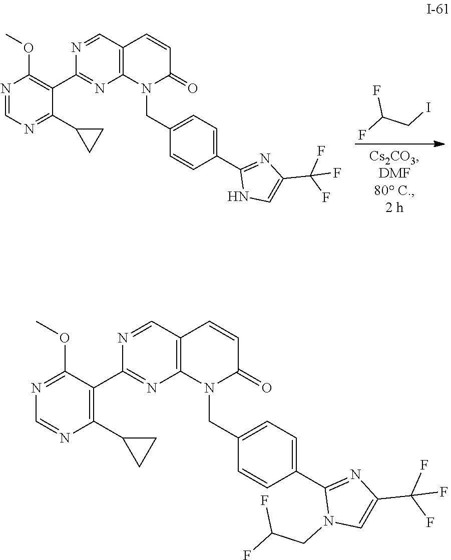 [SMILES: COc1ncnc(C2CC2)c1-c1ncc2ccc(=O)n(Cc3ccc(-c4nc(C(F)(F)F)c[nH]4)cc3)c2n1 ; FC(F)CI ; COc1ncnc(C2CC2)c1-c1ncc2ccc(=O)n(Cc3ccc(-c4nc(C(F)(F)F)cn4CC(F)F)cc3)c2n1]

To a stirred mixture of 2-(4-cyclopropyl-6-methoxypyrimidin-5-yl)-8-({4-[4-(trifluoromethyl)-1H-imidazol-2-yl]phenyl}methyl)pyrido[2,3-d]pyrimidin-7-one (50 mg, 0.096 mmol, 1 equiv) and Cs₂CO₃ (94.08 mg, 0.288 mmol, 3 equiv) in DMF (1 mL, 12.922 mmol, 134.25 equiv) was added 1,1-difluoro-2-iodoethane (73.90 mg, 0.384 mmol, 4 equiv) dropwise at room temperature under nitrogen atmosphere. The resulting mixture was stirred for 2h at under nitrogen atmosphere. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[1-(2,2-difluoroethyl)-4-(trifluoromethyl)imidazol-2-yl]phenyl}methyl)pyrido[2,3-d]pyrimidin-7-one (40.4 mg, 71.79%) as an off-white solid. ¹H NMR (400 MHz, DMSO-d6) δ 9.29 (s, 1H), 8.69 (s, 1H), 8.16 (d, J=9.5 Hz, 1H), 7.97 (s, 1H), 7.54 (d, J=8.1 Hz, 2H), 7.42 (d, J=8.0 Hz, 2H), 6.90 (d, J=9.5 Hz, 1H), 6.53-6.18 (m, 1H), 5.57 (s, 2H), 4.62-4.47 (m, 2H), 3.82 (s, 3H), 1.72 (dq, J=8.2, 4.2 Hz, 1H), 1.01 (q, J=3.6 Hz, 2H), 0.84-0.72 (m, 2H). LC-MS (ESI) m/z 584.2 [M+H].

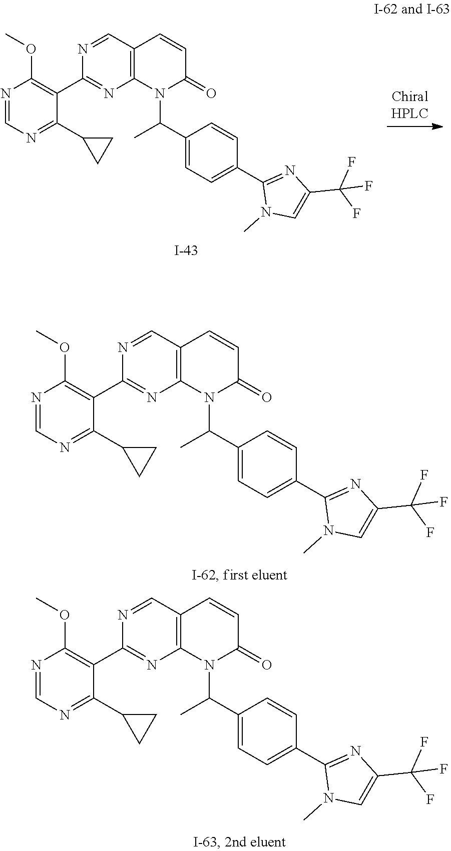

The racemic (2-(4-cyclopropyl-6-methoxypyrimidin-5-yl)-8-(1-{4-[1-methyl-4-(trifluoromethyl)imidazo]-2-yl]phenyl}ethyl)pyrido[2,3-d]pyrimidin-7-one (350 mg) was separated by Chiral-HPLC with the following conditions (Column: (R, R)-WHELK-O, 3*25 cm, 5 μm; Mobile Phase A: Hex(0.5% 2M NH₃-MeOH)-HPLC, Mobile Phase B: IPA-HPLC; Flow rate: 40 mL/min; Gradient: 50% B to 50% B in 35.5 min; Wave Length: 203/260 nm;) to afford enantiomer 1 (1-62) wiretention time 18.5 min and enantiomer 2 (1-63) with retention time 29 min.

1-62: (84.9 mg, 24.01%) as an off-white solid with ee=100% (retention time 1.385 min) Chiral-HPLC conditions: Column Name:(R,R)Whelk-01, 0.46*5 cm, 3.5 um, Mobile Phase :Hex(0.1% DEA):IPA=50:50) ¹H NMR (400 MHz, DMSO-d6) δ 9.26 (s, 1H), 8.68 (s, 1H), 8.11 (d, J=9.4 Hz, 1H), 7.91 (s, 1H), 7.59 (d, J=8.0 Hz, 2H), 7.37 (d, J=8.0 Hz, 2H), 6.81 (s, 2H), 3.82 (s, 3H), 3.73 (s, 3H), 1.94 (d, J=7.0 Hz, 3H), 1.75 (s, 1H), 1.23 (td, J=12.0, 10.9, 5.1 Hz, 2H), 1.01 (s, 2H), 0.81 (s, 2H). LC-MS (ESI) m/z 548.2 [M+H].
1-63 (66.1 mg, 18.75%) as an off-white solid with ee=100% (retention time 1.808 min) Chiral-HPLC conditions: Column Name:(R,R)Whelk-01, 0.46*5 cm, 3.5 um, Mobile Phase :Hex(0.1% DEA):IPA=50:50). ¹H NMR (400 MHz, DMSO-d6) δ 9.25 (s, 1H), 8.68 (s, 1H), 8.11 (d, J=9.4 Hz, 1H), 7.90 (s, 1H), 7.59 (d, J=8.0 Hz, 2H), 7.37 (d, J=8.0 Hz, 2H), 6.82 (s, 2H), 3.82 (s, 3H), 3.73 (s, 3H), 1.94 (d, J=7.0 Hz, 3H), 1.75 (s, 1H), 1.01 (s, 2H), 0.81 (s, 2H). LC-MS: MS (ESI) m/z 548.2 [M+H]

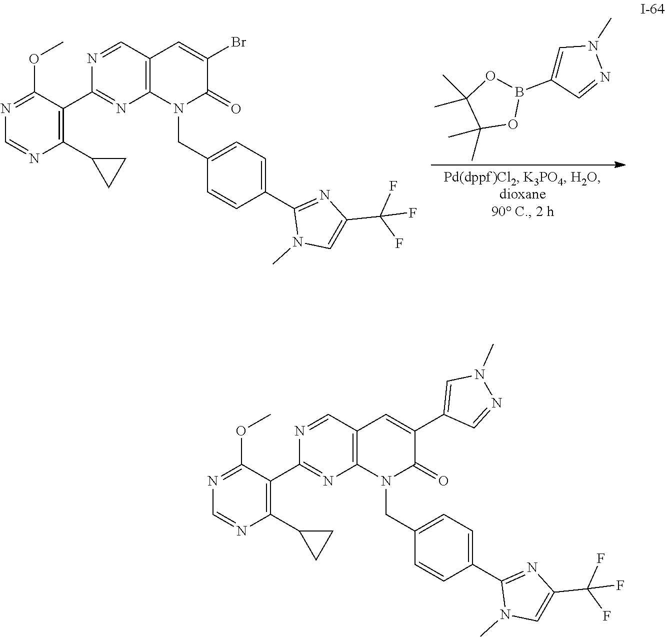

A solution of 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (50 mg, mmol, 1 equiv) in dioxane (2 mL) and H₂O (0.2 mL) was treated with 1-methyl-4-(4,4,5,5-tetramethyl-1,3,2-dioxaborolan-2-yl)pyrazole (20.39 mg, 0.098 mmol, 1.2 equiv), K₃PO₄ (43.33 mg, 0.205 mmol, 2.5 equiv) and Pd(dppf)Cl₂ (5.97 mg, 0.008 mmol, 0.1 equiv) for 2 h at 90° C. under nitrogen atmosphere. The resulting mixture was filtered; the filter cake was washed with MeOH (3×5 mL). The filtrate was concentrated under reduced pressure. The residue was purified by reverse flash chromatography (C18 silica gel column, eluting with 0% to 50% ACN/water (0.1% FA) in 15 min with UV detector (254 nm)) to afford 2-(4-cyclopropyl-6-methoxypyrimidin-yl)pyrido[2,3-d]pyrimidin-7-one (35 mg, 69.65%) as an orange solid. ¹H NMR (400 MHz, DMSO-d 6) δ 9.24 (d, J=1.2 Hz, 1H), 8.69 (d, J=1.3 Hz, 1H), 8.50 (d, J=3.4 Hz, 2H), 8.15 (s, 1H), 7.90 (d, J=1.6 Hz, 1H), 7.63 (d, J=8.0 Hz, 2H), 7.44 (d, J=8.0 Hz, 2H), 5.68 (s, 2H), 3.91 (s, 3H), 3.84 (s, 3H), 3.73 (s, 3H), 1.77 (s, 1H), 1.02 (s, 2H), 0.78 (d, J=6.8 Hz, 2H). LC MS (ESI) m/z 614.25 [M+H].

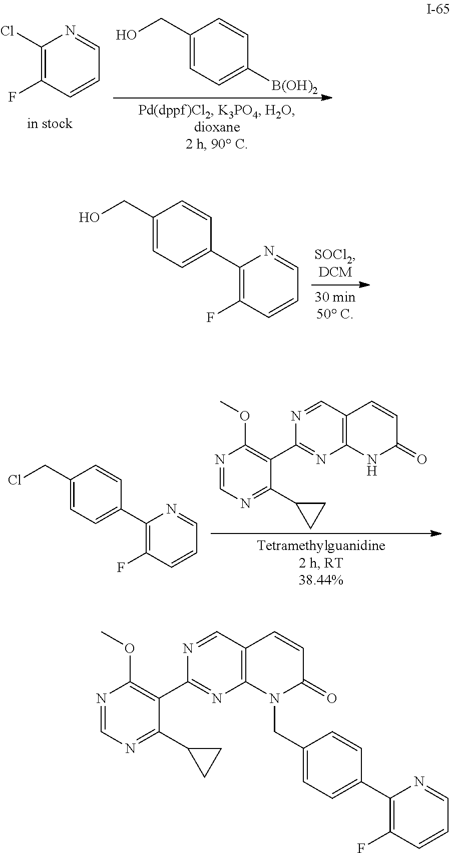

### Step 1: 14-(3-Fluoropyridin-2-Yl)Phenyl]Methanol

To a stirred mixture of 2-chloro-3-fluoropyridine (200 mg, 1.521 mmol, 1 equiv) and 4-(hydroxymethyl)phenylboronic acid (277.28 mg, 1.825 mmol, 1.2 equiv) in dioxane (4 mL, 47.216 mmol, 31.05 equiv) and H₂O (1 mL, 55.509 mmol, 36.51 equiv) were added K₃PO₄ (806.91 mg, 3.802 mmol, 2.5 equiv) and Pd(dppf)Cl₂ (222.52 mg, 0.304 mmol, 0.2 equiv) at room temperature under nitrogen atmosphere. The resulting mixture was stirred for additional 2 h at resulting mixture was filtered; the filter cake was washed with 1,4-dioxane (2×2 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)).to afford [4-(3-fluoropyridin-2-yl)phenyl]methanol (268 mg, 86.73%) as a yellow oil. LC-MS (ESI) m/z 204 [M+H]

### Step 2. 2-[4-(Chloromethyl)Phenyl]-3-Fluoropyridine

To a stirred mixture of [4-(3-fluoropyridin-2-yl)phenyl]methanol (268 mg, 1.319 mmol, 1 equiv) in DCM (4.12 mL, 64.868 mmol, 49.18 equiv) was added SOCl₂ (0.19 mL, 2.638 mmol, 2 equiv) dropwise at room temperature under nitrogen atmosphere. The resulting mixture was stirred for additional 1h at 50° C. The reaction was quenched with Water at room temperature. The mixture basified to pH 7 with saturated NaHCO₃(aq.). The resulting mixture was extracted with CH₂Cl₂ (2×5 mL). The combined organic layers were washed with brine (2×mL), dried over anhydrous Na₂SO₄, filtered and concentrated under reduced pressure. The resulting mixture was used in the next step directly without further purification. LC-MS (ESI) m/z 222 [M+H]

### Step 3: 2-(4-Cyclopropyl-6-Methoxypyrimidin-5-Yl)-8-{[4-(3-Fluoropyridin-2-Yl)Phenyl}Methyl}Pyrido[2,3-d]Pyrimidin-7-One

To a stirred mixture of 2-[4-(chloromethyl)phenyl]-3-fluoropyridine (60 mg, 0.271 mmol, 1 equiv) and 2-(4-cyclopropyl-6-methoxypyrimidin-5-yl)-8H-pyrido[2,3-d]pyrimidin-7-one (79.93 mg, 0.271 mmol, 1 equiv) in DMF (1 mL, 12.922 mmol, 47.74 equiv) was added N,N,N′,N′-tetramethylguanidine (46.76 mg, 0.407 mmol, 1.5 equiv) dropwise at room temperature under nitrogen atmosphere. The resulting mixture was stirred for additional 2 h at room temperature. The residue was purified by reversed-phase flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-{[4-(3-fluoropyridin-2-yl)phenyl]methyl}pyrido[2,3-d]pyrimidin-7-one (50 mg, 38.44%) as a white solid. ¹H NMR (400 MHz, DMSO-d6) δ 9.29 (s, 1H), 8.68 (s, 1H), 8.53 (d, J=4.6 Hz, 1H), 8.16 (d, J=9.6 Hz, 1H), 7.85-7.75 (m, 3H), 7.48 (d, J=4.2 Hz, 1H), 7.39 (d, J=8.3 Hz, 2H), 6.91 (d, J=9.5 Hz, 1H), (s, 2H), 3.81 (s, 3H), 1.70 (t, J=4.4 Hz, 1H), 1.02-0.85 (m, 2H), 0.73 (dt, J=8.1, 3.4 Hz, 2H). LC-MS (ESI) m/z 481.20 [M+H]

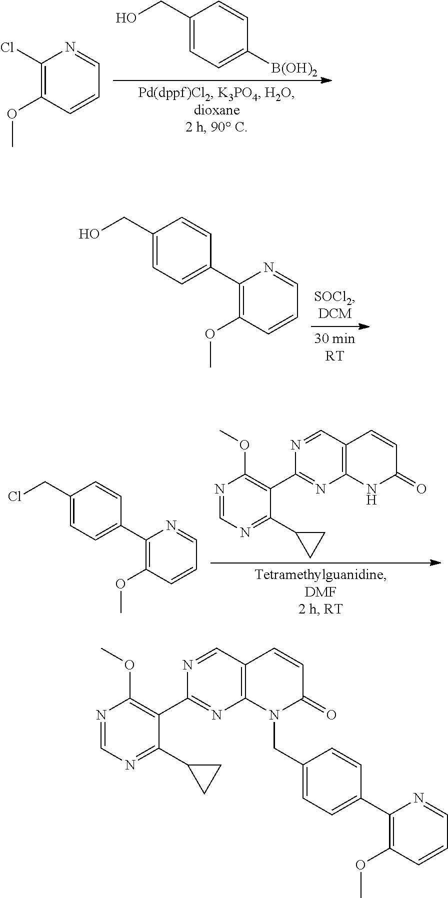

### Step 1: 14-(3-Methoxypyridin-2-Yl)Phenyl]Methanol

To a stirred mixture of 2-chloro-3-methoxypyridine (200 mg, 1.393 mmol, 1 equiv) and 4-(hydroxymethyl)phenylboronic acid (254.03 mg, 1.672 mmol, 1.2 equiv) in dioxane (4 mL, 47.216 mmol, 33.89 equiv) and H₂O (1 mL, 55.509 mmol, 39.85 equiv) were added K₃PO₄ (739.24 mg, 3.482 mmol, 2.5 equiv) and Pd(dppf)Cl₂ (203.86 mg, 0.279 mmol, 0.2 equiv) at room temperature under nitrogen atmosphere. The resulting mixture was stirred for additional 2h at resulting mixture was concentrated under reduced pressure. The residue was purified by reversed-phase flash chromatography (C18 silica gel column, eluting with 0% to 100% MeCN/water (0.1% FA) in 10 min with UV detector (254 nm)) to afford [4-(3-methoxypyridin-2-yl)phenyl]methanol (260 mg, 86.71%) as a yellow oil. LC-MS (ESI) m/z 216 [M+H]

### Step 2: 2-[4-(Chloromethyl)Phenyl]-3-Methoxypyridine

To a stirred mixture of [4-(3-methoxypyridin-2-yl)phenyl]methanol (65 mg, 0.302 mmol, 1 equiv) in DCM (1 mL, 15.731 mmol, 52.09 equiv) was added SOCl₂ (0.04 mL, 0.604 mmol, 2 equiv) dropwisesn at room temperature under nitrogen atmosphere. The reaction was quenched with Water at room temperature. The mixture basified to pH 7 with saturated NaHCO₃(aq.). The resulting mixture was extracted with CH₂Cl₂ (2×3 mL). The combined organic layers were washed with brine (2×3 mL), dried over anhydrous Na₂SO₄, filtered and concentrated under reduced pressure. The resulting mixture was used in the next step directly without further purification. LC-MS (ESI) m/z 234 [M+H]

### Step 3: 2-(4-Cyclopropyl-6-Methoxypyrimidin-5-Yl)-8-{14-(3-Methoxypyridin-2-Yl)Phenyl]Methyl}Pyrido[2,3-d]Pyrimidin-7-One

To a stirred mixture of 2-[4-(chloromethyl)phenyl]-3-methoxypyridine (60 mg, mmol, 1 equiv) and 2-(4-cyclopropyl-6-methoxypyrimidin-5-yl)-8H-pyrido[2,3-d]pyrimidin-7-one (75.82 mg, 0.257 mmol, 1 equiv) in DMF (1 mL, 12.922 mmol, 50.33 equiv) was added N,N,N′,N′-tetramethylguanidine (44.36 mg, 0.386 mmol, 1.5 equiv) dropwise at room temperature under nitrogen atmosphere. The resulting mixture was stirred for additional 2 h at room temperature. The residue was purified by reversed-phase flash chromatography (C18 silica gel column, eluting with 0% to 100% MeCN/water (0.1% FA) in 10 min with UV detector (254 nm)). to afford 2-(4-cyclopropyl-6-methoxypyrimidin-5-yl)-8-{[4-(3-methoxypyridin-2-yl)phenyl]methyl}pyrido[2,3-d]pyrimidin-7-one (60 mg, 47.45%) as a white solid. ¹H NMR (400 MHz, DMSO-d6) δ 9.28 (s, 1H), 8.69 (s, 1H), 8.22 (dd, J=4.6, 1.3 Hz, 1H), 8.15 (d, J=9.5 Hz, 1H), 7.79-7.70 (m, 2H), 7.53 (dd, J=8.5, 1.3 Hz, 1H), 7.36-7.24 (m, 3H), 6.90 (d, J=9.5 Hz, 1H), 5.55 (s, 2H), 3.81 (d, J=7.5 Hz, 6H), 1.71 (dt, J=8.0, 3.6 Hz, 1H), 1.00 (s, 2H), 0.76 (dd, J=8.0, 3.3 Hz, 2H). LC-MS (ESI) m/z 493.25 [M+H]

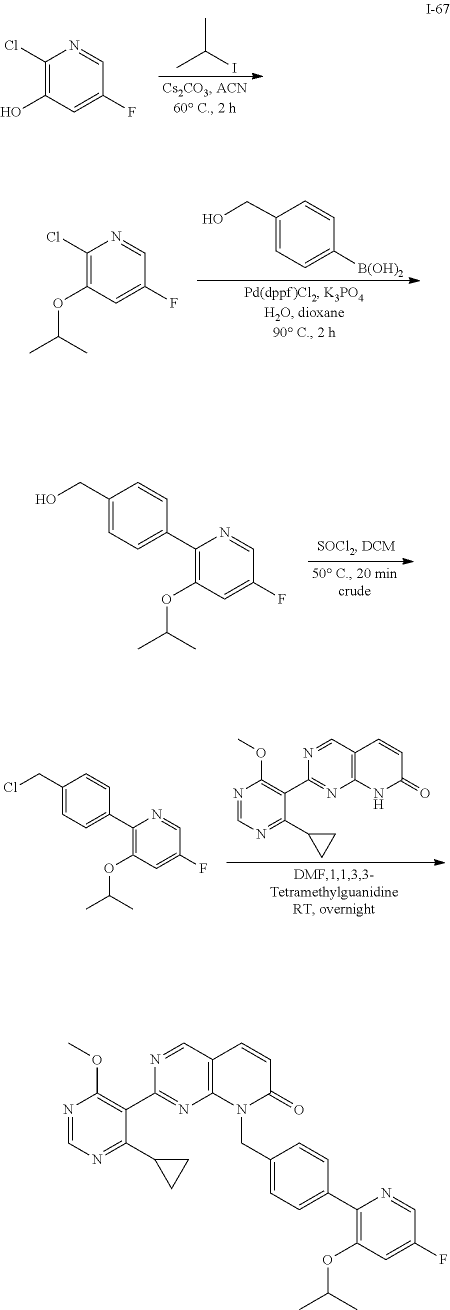

### Step 1: 2-Chloro-5-Fluoro-3-Isopropoxypyridine

A solution of 2-chloro-5-fluoropyridin-3-ol (441 mg, 2.989 mmol, 1 equiv) in ACN (6 mL, 114.145 mmol, 38.19 equiv) was treated with 2-iodopropane (762.22 mg, 4.483 mmol, 1.5 equiv) and Cs₂CO₃ (1947.89 mg, 5.978 mmol, 2 equiv) for 2 h at 60° C. under nitrogen atmosphere. The resulting mixture was filtered, the filter cake was washed with EA (3×10 mL). The filtrate was concentrated under reduced pressure. The residue was purified by silica gel column chromatography, (eluting with PE/EA (1:1)) to afford 2-chloro-5-fluoro-3-isopropoxypyridine (500 mg, 88.22%) as a light yellow liquid. LCMS (ESI) m/z 190.04 [M+H].

### Step 2: [4-(5-Fluoro-3-Isopropoxypyridin-2-Yl)Phenyl]Methanol

A solution of 2-chloro-5-fluoro-3-isopropoxypyridine (189 mg, 0.997 mmol, 1 equiv) in dioxane (0.4 mL, 4.722 mmol, 4.74 equiv) and H₂O (4 mL, 222.037 mmol, 222.75 equiv) was treated with 4-(hydroxymethyl)phenylboronic acid (181.77 mg, 1.196 mmol, 1.2 equiv), K₃PO₄ (528.96 mg, 2.493 mmol, 2.5 equiv) and Pd(dppf)Cl₂ (72.94 mg, 0.100 mmol, 0.1 equiv) for 2 h at 90° C. under nitrogen atmosphere. The resulting mixture was filtered, the filter cake was washed with MeOH (3×10 mL). The residue was purified by reversed-phase flash chromatography (C18 silica gel column, eluting with 10% to 50% MeCN/water (0.1% FA) in 10 min with UV detector (254 nm)). The resulting mixture was concentrated under reduced pressure to afford [4-(5-fluoro-3-isopropoxypyridin-2-yl)phenyl]methanol (240 mg, 92.15%) as a yellow liquid. LCMS (ESI) m/z 262.12 [M+H].

### Step 3: 2-[4-(Chloromethyl)Phenyl]-5-Fluoro-3-Isopropoxypyridine

A solution of [4-(5-fluoro-3-isopropoxypyridin-2-yl)phenyl]methanol (120 mg, mmol, 1 equiv) in DCM (2 mL) was treated with SOCl₂ (0.10 mL, 1.377 mmol, 3 equiv) for at 50° C. under nitrogen atmosphere. The reaction was quenched by the addition of Water/Ice (10 mL) at 0° C. The resulting mixture was extracted with CH₂Cl₂ (3×10 mL). The combined organic layers were washed with sat. NaCl (aq.) (3×10 mL), dried over anhydrous Na₂SO₄, filtered and concentrated under reduced pressure to afford 2-[4-(chloromethyl)phenyl]-5-fluoro-3-isopropoxypyridine (100 mg, crude) as a yellow solid. The crude product was used in the next step directly without further purification. LCMS (ESI) m/z 280.08 [M+H].

### Step 4: 2-(4-Cyclopropyl-6-Methoxypyrimidin-5-Yl)-8-{14-(5-Fluoro-3-Isopropoxypyridin-2-Yl)Phenyl]Methyl}Pyrido[2,3-d]Pyrimidin-7-One

A solution of 2-[4-(chloromethyl)phenyl]-5-fluoro-3-isopropoxypyridine (66 mg, mmol, 0.9 equiv) in DMF (2 mL) was treated with 2-(4-cyclopropyl-6-methoxypyrimidin-(77.41 mg, 0.262 mmol, 1 equiv) and 1,1,3,3-Tetramethylguanidine (45.29 mg, 0.393 mmol, 1.5 equiv) for overnight at room temperature under nitrogen atmosphere. The resulting mixture was diluted with DMF (2 mL). The residue was purified by reverse flash chromatography (C18 silica gel column, eluting with 0% to 50% ACN/water (0.1% FA) in 15 min with UV detector (254 nm)) to afford 2-(4-cyclopropyl-6-methoxypyrimidin-5-yl)-8-{[4-(5-fluoro-3-isopropoxypyridin-2-yl)phenyl]methyl}pyrido[2,3-d]pyrimidin-7-one (11.0 mg, 7.75%) as an off-white solid. ¹H NMR (400 MHz, DMSO-d6) δ 9.29 (s, 1H), 8.68 (s, 1H), 8.21 (d, J=2.3 Hz, 1H), 8.16 (d, J=9.5 Hz, 1H), 7.77 (d, J=8.2 Hz, 2H), 7.60 (dd, J=11.3, 2.3 Hz, 1H), 7.30 (d, J=8.2 Hz, 2H), 6.90 (d, J=9.4 Hz, 1H), 5.54 (s, 2H), 4.81-4.73 (m, 1H), 3.82 (s, 3H), 1.71 (d, J=4.2 Hz, 1H), 1.26 (d, J=6.0 Hz, 6H), 0.99 (s, 2H), (dd, J=8.1, 3.3 Hz, 2H). LCMS (ESI) m/z 539.30 [M+H].

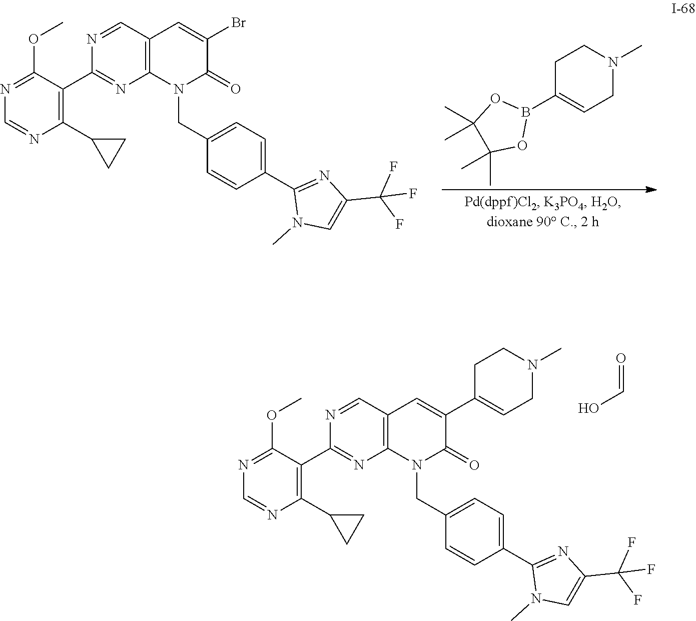

A solution of 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (50 mg, mmol, 1 equiv) in dioxane (2 mL, 23.608 mmol, 289.16 equiv) and H₂O (0.2 mL, 11.102 mmol, 135.98 equiv) was treated with 1-methyl-4-(4,4,5,5-tetramethyl-1,3,2-dioxaborolan-2-yl)-3,6-dihydro-2H-pyridine (21.86 mg, 0.098 mmol, 1.2 equiv), K₃PO₄ (43.33 mg, 0.205 mmol, 2.5 equiv) and Pd(dppf)C12 (5.97 mg, 0.008 mmol, 0.1 equiv) for 2 h at 90° C. under nitrogen atmosphere. The resulting mixture was filtered, the filter cake was washed with MeOH (3×4 mL). The filtrate was concentrated under reduced pressure. The filtrate was concentrated under reduced pressure. The residue was purified by reverse flash chromatography (C18 silica gel column, eluting with 0% to 100% ACN/water (0.1% FA) in 15 min with UV detector (254 nm)) to afford 2-(4-cyclopropyl-6-methoxypyrimidin-5-yl)-6-(1-methyl-3,6-dihydro-2H-pyridin-4-yl)-8-({4-[1-methyl-4-(trifluoromethyl)imidazo]-2-yl]phenyl}methyl)pyrido[2,3-d]pyrimidin-7-, formic acid (26.5 mg, 50.65%) as an orange solid. ¹H NMR (400 MHz, DMSO-d6) δ 9.28 (s, 1H), 8.69 (s, 1H), 8.08 (s, 1H), 7.91 (s, 1H), 7.66-7.59 (m, 2H), 7.41 (d, J=8.1 Hz, 2H), 6.77 (d, J=3.8 Hz, 1H), (s, 2H), 3.84 (s, 3H), 3.74 (s, 3H), 3.08 (s, 2H), 2.58 (d, J=5.1 Hz, 2H), 2.54 (s, 2H), 2.29 (s, 3H), 1.73 (dq, J=8.1, 4.5, 4.0 Hz, 1H), 1.01 (q, J=3.5 Hz, 2H), 0.78 (dd, J=7.9, 3.5 Hz, 2H). LCMS (ESI) m/z 629.25 [M+H].

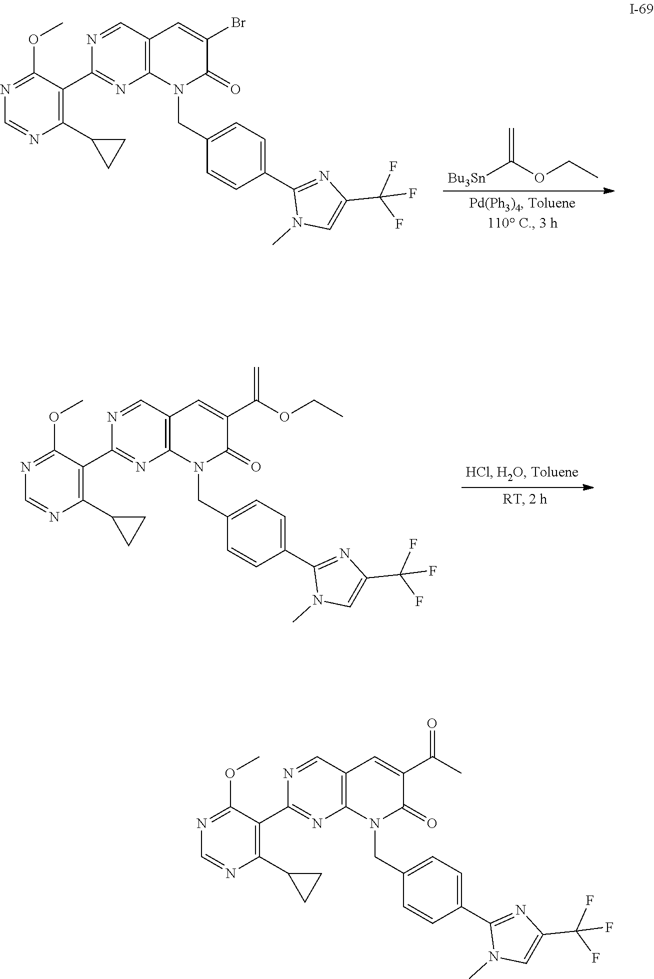

### Step 1: 2-(4-Cyclopropyl-6-Methoxypyrimidin-5-Yl)-6-(1-Ethoxyethenyl)-8-({4-[1-Methyl-4-(Trifluoromethyl)Imidazol-2-Yl]Phenyl}Methyl)Pyrido[2,3-d]Pyrimidin-7-One

To a stirred solution of 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (30 mg, 0.049 mmol, 1.00 equiv) and tributyl(1-ethoxyethenyl)stannane (21.23 mg, 0.059 mmol, 1.2 equiv) in Toluene (4.51 mL) was added Pd(PPh₃)₄ (5.66 mg, 0.005 mmol, 0.1 equiv) at room temperature. The resulting mixture was stirred for 3 h at 110° C. under nitrogen atmosphere. The resulting mixture was concentrated under reduced pressure and used in the next step directly without further purification. LC-MS-(ESI) m/z 604.6 [M+H]

### Step 2: 6-Acetyl-2-(4-Cyclopropyl-6-Methoxypyrimidin-5-Yl)-8-({4-[4-(Difluoromethyl)-1-Methylimidazol-2-Yl]Phenyl}Methyl)Pyrido[2,3-d]Pyrimidin-7-One

A solution of 2-(4-cyclopropyl-6-methoxypyrimidin-5-yl)-6-(1-ethoxyethenyl)-8-({4-[1-methyl-4-(trifluoromethyl)imidazo]-2-yl]phenyl}methyl)pyrido[2,3-d]pyrimidin-7-one (30 mg, 0.050 mmol, 1 equiv) in 2 M HCl (0.2 mL) and Toluene (1 mL) was stirred for 2 h at room temperature. The mixture was neutralized to pH 7 with saturated NaHCO₃(aq.). The resulting mixture was extracted with EA (3×5 mL). The combined organic layers were dried over anhydrous Na₂SO₄, filtered and concentrated under reduced pressure. The residue was purified by reversed-phase flash chromatography (C18 silica gel column, eluting with 0% to 100% MeCN/water (0.1% FA) in 10 min with UV detector (254 nm)) to afford 6-acetyl-2-(4-cyclopropyl-6-methoxypyrimidin-5-yl)-8-({4-[4-(difluoromethyl)-1-methylimidazol-2-yl]phenyl}methyl)pyrido[2,3-d]pyrimidin-7-one (9.1 mg, 32.71%) as an off-white solid. ¹H NMR (400 MHz, DMSO-d6) δ 9.48 (d, J=1.1 Hz, 1H), 8.68 (d, J=11.6 Hz, 2H), 7.91 (t, J=1.3 Hz, 1H), 7.64 (d, J=8.1 Hz, 2H), 7.44 (d, J=8.1 Hz, 2H), 5.61 (s, 2H), 3.84 (d, J=1.0 Hz, 3H), 3.74 (s, 3H), 2.64 (d, J=1.1 Hz, 3H), 1.76 (tt, J=8.1, 4.6 Hz, 1H), 1.07-0.99 (m, 2H), 0.78 (dq, J=3.5 Hz, 2H). LC-MS (ESI) m/z 576.05 [M+H]

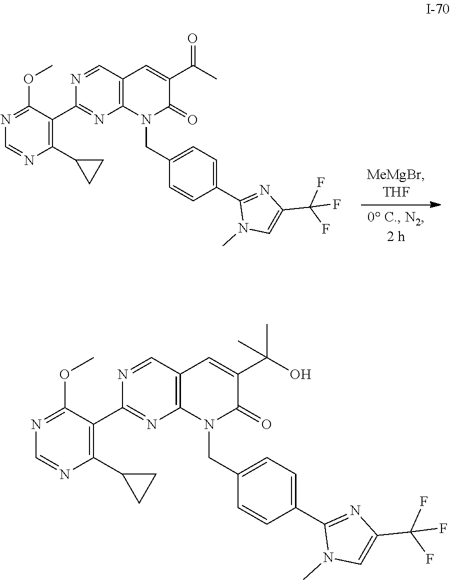 [SMILES: COc1ncnc(C2CC2)c1-c1ncc2cc(C(C)=O)c(=O)n(Cc3ccc(-c4nc(C(F)(F)F)cn4C)cc3)c2n1 ; COc1ncnc(C2CC2)c1-c1ncc2cc(C(C)(C)O)c(=O)n(Cc3ccc(-c4nc(C(F)(F)F)cn4C)cc3)c2n1]

To a stirred solution of 6-acetyl-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (80 mg, 0.139 mmol, 1 equiv) in THF (2 mL) was added MeMgBr (0.15 mL, 3.0 equiv, 3M in THF) dropwise at 0° C. under nitrogen atmosphere. The resulting mixture was stirred for 2 h at 0° C. under nitrogen atmosphere. The reaction was quenched by the addition of sat. NH₄Cl (aq.) (2 mL) at room temperature. The resulting mixture was extracted with ethyl acetate (3×10 mL). The combined organic layers were dried over anhydrous Na₂SO₄ and concentrated under reduced pressure. The crude product was purified by Prep-HPLC with the following conditions (Column: YMC-Actus Triart C18 ExRS, 30*150 mm, 51 μm; Mobile Phase A: Water(10 mmol/L NH₄HCO₃), Mobile Phase B: ACN; Flow rate: 60 mL/min; Gradient: 39% B to 55% B in 9 min, 55% B; Wave Length: 254/220 nm; RT1(min): 9.67) to afford 2-(4-cyclopropyl-6-methoxypyrimidin-5-yl)-6-(2-hydroxypropan-2-yl)-8-({4-[1-methyl-4-(trifluoromethyl)imidazo]-2-yl]phenyl}methyl)pyrido[2,3-d]pyrimidin-7-one (24.8 mg, 28.65%) as an off-white solid. ¹H NMR (400 MHz, DMSO-d6) δ 9.34 (d, J=1.2 Hz, 1H), 8.69 (d, J=1.2 Hz, 1H), 8.30 (d, J=1.3 Hz, 1H), 7.90 (t, J=1.4 Hz, 1H), 7.66-7.59 (m, 2H), 7.40 (d, J=8.2 Hz, 2H), 5.60 (s, 2H), 5.45 (s, 1H), 3.83 (d, J=1.2 Hz, 3H), 3.74 (s, 3H), 1.73 (td, J=8.8, 8.1, 4.8 Hz, 1H), 1.54 (s, 6H), 1.01 (d, J=4.2 Hz, 2H), 0.77 (dd, J=8.0, 3.3 Hz, 2H). LC-MS-(ESI) m/z 592.10 [M+H].

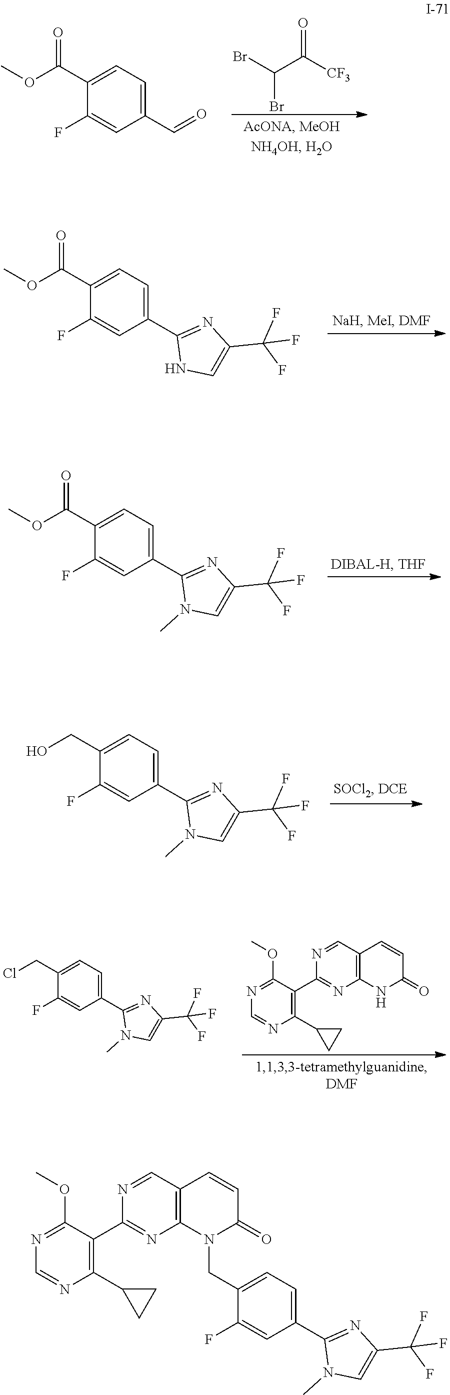

### Step 1: Methyl 2-Fluoro-4-[4-(Trifluoromethyl)-1H-Imidazol-2-Yl]Benzoate

To a stirred solution of 3,3-dibromo-1,1,1-trifluoropropan-2-one (331.84 mg, 1.230 mmol, 1.12 equiv) in H₂O (0.4 mL, 22.204 mmol, 20.22 equiv) were added AcONa (73.85 mg, 1.230 mmol, 1.12 equiv) in portions at room temperature under nitrogen atmosphere. The resulting mixture was stirred for additional 1 h at 100° C. The mixture was allowed to cool down to room temperature. To the above mixture was added MeOH (3 mL, 74.096 mmol, 67.48 equiv), NH 4 OH (0.75 mL, 19.260 mmol, 17.54 equiv) and methyl 2-fluoro-4-formylbenzoate (200 mg, 1.098 mmol, 1 equiv) in portions at room temperature. The resulting mixture was stirred for additional min at room temperature. Then the resulting mixture was stirred for 2 h at 100° C. under nitrogen atmosphere. The resulting mixture was extracted with EtOAc (3×20 mL). The combined organic layers were washed with brine (3×10 mL), dried over anhydrous Na₂SO₄, filtered and concentrated under reduced pressure. The residue was purified by silica gel column chromatography (eluting with PE/EA (1:1)) to afford methyl 2-fluoro-4-[4-(trifluoromethyl)-1H-imidazol-2-yl]benzoate (192.1 mg, 60.71%) as a yellow solid. LC-MS m/z 289 [M+H]

### Step 2: Methyl 2-Fluoro-4-[1-Methyl-4-(Trifluoromethyl)Imidazol-2-Yl]Benzoate

To a stirred solution of methyl 2-fluoro-4-[4-(trifluoromethyl)-1H-imidazol-2-yl]benzoate (192 mg, 0.666 mmol, 1 equiv) and DMF (4 mL, 51.686 mmol, 77.58 equiv) were added NaH (31.97 mg, 1.332 mmol, 2 equiv) in portions at 0° C. under nitrogen atmosphere. The resulting mixture was stirred for additional 30 min at 0° C. To the above mixture was added MeI (283.68 mg, 1.998 mmol, 3 equiv) dropwise at 0° C. The resulting mixture was stirred for additional 2 h at room temperature. The reaction was quenched with Water at 0° C. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% TFA) with UV detector (254 nm)) to yield methyl 2-fluoro-4-[1-methyl-4-(trifluoromethyl)imidazol-2-yl]benzoate (170 mg, 84.43%) as a yellow solid. LC-MS (ESI) m/z 303 [M+H].

### Step 3: 2-Fluoro-4-[1-Methyl-4-(Trifluoromethyl)Imidazol-2-Yl]Phenyl}Methanol

To a stirred solution of methyl 2-fluoro-4-[1-methyl-4-(trifluoromethyl)imidazo]-2-yl]benzoate (170 mg, 0.562 mmol, 1 equiv) and THF (3 mL, 37.028 mmol, 65.83 equiv) were added DIBAL-H (1.87 mL, 2.810 mmol, 5 equiv) dropwise at 0° C. under nitrogen atmosphere. The resulting mixture was stirred for additional 50 min at room temperature. The reaction was quenched with Water at 0° C. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% TFA) with UV detector (254 nm)) to yield {2-fluoro-4-[1-methyl-4-(trifluoromethyl)imidazo]-2-yl]phenyl}methanol (120 mg, 77.80%) as a colorless solid. LC-MS (ESI) m/z 275 [M+H]

### Step 4: 2-[4-(Chloromethyl)-3-Fluorophenyl]-1-Methyl-4-(Trifluoromethyl)Imidazole

To a stirred solution of {2-fluoro-4-[1-methyl-4-(trifluoromethyl)imidazo]-2-yl]phenyl}methanol (50 mg, 0.182 mmol, 1 equiv) and DCE (1 mL, 12.633 mmol, 69.28 equiv) were added SOCl₂ (0.04 mL, 0.546 mmol, 3 equiv) dropwise at room temperature under nitrogen atmosphere. The resulting mixture was stirred for additional 30 min at 50° C. The reaction was quenched with Water at 0° C. The mixture was neutralized to pH 7 with saturated NaHCO₃(aq.). The resulting mixture was extracted with DCM (3×20 mL). The combined organic layers were washed with brine (3×10 mL), dried over anhydrous Na₂SO₄, filtered and concentrated under reduced pressure to afford 2-[4-(chloromethyl)-3-fluorophenyl]-1-methyl-4-(trifluoromethyl)imidazole (83.2 mg, crude) as a colorless solid. The crude product mixture was used in the next step directly without further purification. LC-MS (ESI) m/z 293 [M+H]

### Step 5: 2-(4-Cyclopropyl-6-Methoxypyrimidin-5-Yl)-8-({2-Fluoro-4-[1-Methyl-4-(Trifluoromethyl)Imidazol-2-Yl]Phenyl}Methyl)Pyrido[2,3-d]Pyrimidin-7-One

To a stirred solution of 2-[4-(chloromethyl)-3-fluorophenyl]-1-methyl-4-(trifluoromethyl)imidazole (83 mg, 0.284 mmol, 1 equiv) and 2-(4-cyclopropyl-6-methoxypyrimidin-5-yl)-8H-pyrido[2,3-d]pyrimidin-7-one (83.75 mg, 0.284 mmol, 1 equiv) in DMF (1 mL, 12.922 mmol, 45.56 equiv) were added 1,1,3,3-tetramethylguanidine (49.00 mg, mmol, 1.5 equiv) dropwise at room temperature under nitrogen atmosphere. The resulting mixture was stirred for additional 3 h at room temperature. The resulting mixture was purified by reverse flash chromatography (C18 silica gel column, eluting with 0% to 100% in 10 min MeCN/water (0.1% FA) with UV detector (254 nm)) to 2-(4-cyclopropyl-6-methoxypyrimidin-5-yl)-8-({2-fluoro-4-[1-methyl-4-(trifluoromethyl)imidazo]-2-yl]phenyl}methyl)pyrido[2,3-d]pyrimidin-7-one (64.2 mg, 40.64%) as a brown yellow solid. ¹H NMR (400 MHz, DMSO-d6) δ 9.31 (s, 1H), 8.67 (s, 1H), 8.19 (d, J=9.5 Hz, 1H), 7.94 (s, 1H), 7.57 (dd, J=11.2, 1.7 Hz, 1H), 7.41 (dd, J=8.0, 1.7 Hz, 1H), 7.09 (t, J=8.0 Hz, 1H), 6.92 (d, J=9.6 Hz, 1H), 5.59 (s, 2H), 3.77 (d, J=7.5 Hz, 6H), 1.72 (tt, J=8.2, 4.6 Hz, 1H), 0.98 (p, J=3.8 Hz, 2H), 0.72 (dq, J=6.9, 3.5 Hz, 2H). LC-MS (ESI) m/z 552 [M+H].

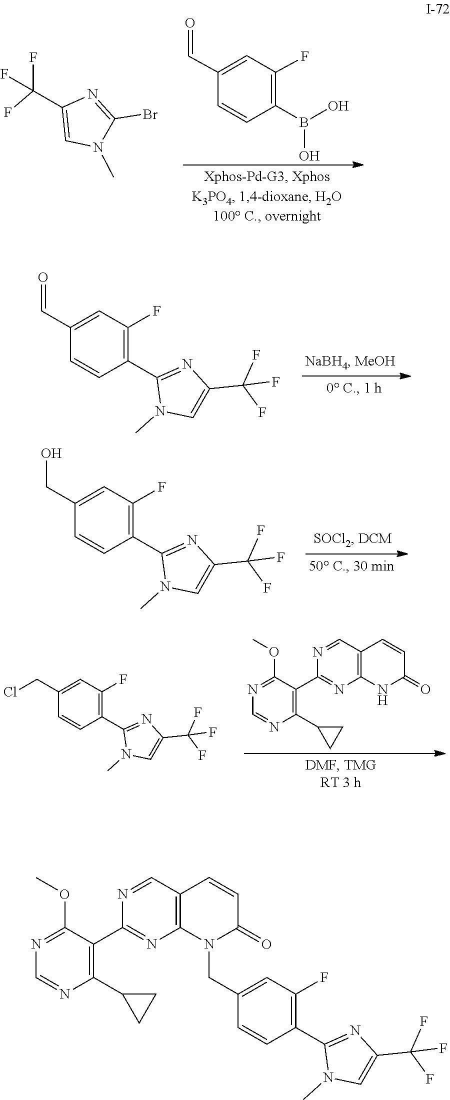

### Step 1 3-Fluoro-4-[1-Methyl-4-(Trifluoromethyl)Imidazol-2-Yl]Benzaldehyde

To a stirred mixture of 2-bromo-1-methyl-4-(trifluoromethyl)imidazole (100 mg, mmol, 1 equiv) and 2-fluoro-4-formylphenylboronic acid (88.00 mg, 0.524 mmol, 1.2 equiv) in H₂O (0.4 mL, 4 equiv) and 1,4-dioxane (1.6 mL, 16 equiv) were added xphos (83.27 mg, mmol, 0.4 equiv), K₃PO₄ (278.08 mg, 1.311 mmol, 3 equiv) and XPhos Pd G3 (36.96 mg, mmol, 0.1 equiv) at room temperature under nitrogen atmosphere. The resulting mixture was stirred for overnight at 100° C. under nitrogen atmosphere. The resulting mixture was filtered; the filter cake was washed with MeOH (3×1 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford 3-fluoro-4-[1-methyl-4-(trifluoromethyl)imidazol-2-yl]benzaldehyde (28 mg, 23.56%) as a colorless oil. LC-MS (ESI) m/z 273.1 [M+H]

### Step 2 {3-Fluoro-4-[1-Methyl-4-(Trifluoromethyl)Imidazol-2-Yl]Phenyl}Methanol

To a stirred 3-fluoro-4-[1-methyl-4-(trifluoromethyl)imidazo]-2-yl]benzaldehyde (75 mg, 0.276 mmol, 1 equiv) in MeOH (1.5 mL, 20 equiv) was added NaBH 4 (15.63 mg, 0.414 mmol, 1.5 equiv) at 0° C. under nitrogen atmosphere. The resulting mixture was stirred for 1 h at under nitrogen atmosphere. The reaction was quenched with water at 0° C. The residue was purified by reverse flash chromatography (C18 silica gel column, eluting with 0% to 100% MeCN/water (0.1% FA) in 10 min with UV detector (254 nm)) to afford {3-fluoro-4-[1-methyl-4-(trifluoromethyl)imidazo]-2-yl]phenyl}methanol (48 mg, 63.53%) as a colorless oil. LC-MS (ESI) m/z 275.1 [M+H]

### Step 3: 2-[4-(Chloromethyl)-2-Fluorophenyl]-1-Methyl-4-(Trifluoromethyl)Imidazol

To a stirred {3-fluoro-4-[1-methyl-4-(trifluoromethyl)imidazol-2-yl]phenyl}methanol (50 mg, 0.182 mmol, 1 equiv) in DCM (1 mL, 20 equiv) were added SOCl₂ (65.07 mg, 0.546 mmol, 3.0 equiv) at room temperature under nitrogen atmosphere. The resulting mixture was stirred for 30 min at 50° C. under nitrogen atmosphere. The reaction was quenched with water at 0° C. The residue was neutralized to pH 7 with saturated NaHCO₃(aq.). The resulting mixture was extracted with CH₂Cl₂ (3×3 mL). The combined organic layers were washed with brine (2×2 mL), dried over anhydrous Na₂SO₄, filtered and concentrated under reduced pressure to afford 2-[4-(chloromethyl)-2-fluorophenyl]-1-methyl-4-(trifluoromethyl)imidazole (50 mg, 93.70%) as a yellow solid. The crude resulting mixture was used in the next step directly without further purification. LC-MS(ESI) m/z 293.1 [M+H]

### Step 4 2-(4-Cyclopropyl-6-Methoxypyrimidin-5-Yl)-8-({3-Fluoro-4-[1-Methyl-4-(Trifluoromethyl)Imidazol-2-Yl]Phenyl}Methyl)Pyrido[2,3-d]Pyrimidin-7-One

To a stirred mixture of 2-[4-(chloromethyl)-2-fluorophenyl]-1-methyl-4-(trifluoromethyl)imidazole (45 mg, 0.154 mmol, 1 equiv) and 2-(4-cyclopropyl-6-methoxypyrimidin-5-yl)-8H-pyrido[2,3-d]pyrimidin-7-one (45.41 mg, 0.154 mmol, 1.0 equiv) in DMF (0.9 mL, 20 equiv) was added N,N,N′,N′-tetramethylguanidine (26.56 mg, 0.231 mmol, 1.5 equiv) at room temperature under nitrogen atmosphere. The resulting mixture was stirred for 3h at room temperature under nitrogen atmosphere. The crude product (50 mg) was purified by Prep-HPLC with the following conditions (Column: Xselect CSH C18 OBD Column 51 μm, n; Mobile Phase A: Water(0.1% FA), Mobile Phase B: ACN; Flow rate: 60 mL/min; Gradient: 36% B to 53% B in 8 min, 53% B; Wave Length: 254/220 nm; RT1(min): 7.33;) to afford 2-(4-cyclopropyl-6-methoxypyrimidin-5-yl)-8-(3-fluoro-4-[1-methyl-4-(trifluoromethyl)imidazo]-2-yl]phenyl methyl)pyrido[2,3-d]pyrimidin-7-one (33.7 mg, 39.70%) as a white solid. ¹H NMR (400 MHz, DMSO-d6) δ 9.29 (s, 1H), 8.69 (s, 1H), 8.17 (d, J=9.5 Hz, 1H), 7.98 (d, J=1.5 Hz, 1H), 7.51 (t, J=7.8 Hz, 1H), 7.31 (d, J=11.1 Hz, 1H), 7.25 (dd, J=7.8, 1.6 Hz, 1H), 6.90 (d, J=9.6 Hz, 1H), 5.57 (s, 2H), 3.82 (s, 3H), 3.56 (d, J=1.5 Hz, 3H), 1.74 (tt, J=8.3, 4.6 Hz, 1H), 1.02 (p, J=3.7 Hz, 2H), 0.78 (dq, J=6.9, 3.5 Hz, 2H). LC-MS (ESI) m/z 552.3 [M+H].

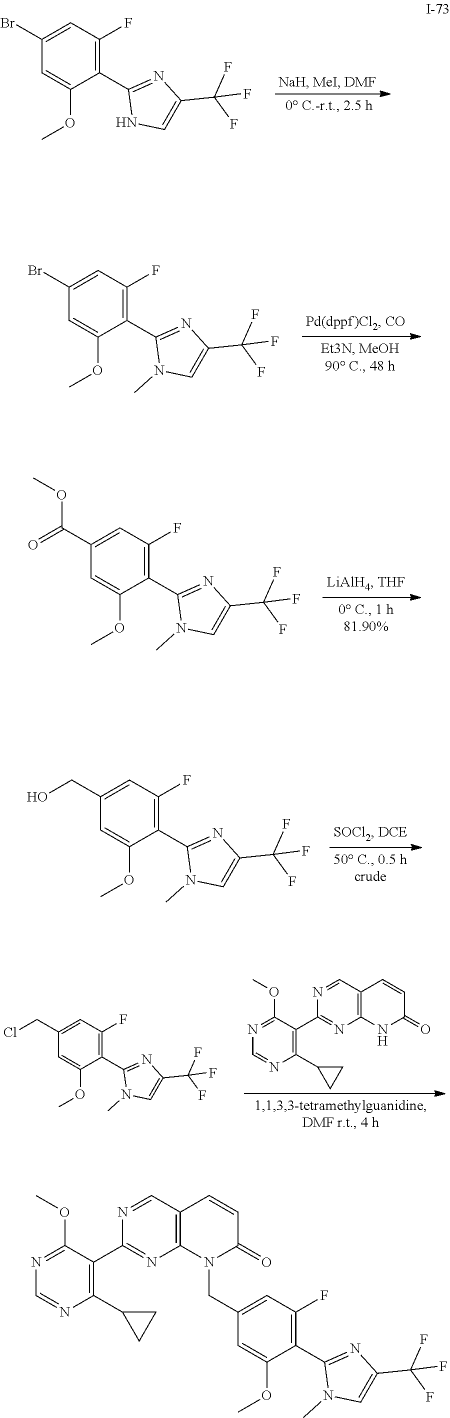

### Step 1: 2-(4-Bromo-2-Fluoro-6-Methoxyphenyl)-1-Methyl-4-(Trifluoromethyl)Imidazole

To a stirred solution of 2-(4-bromo-2-fluoro-6-methoxyphenyl)-4-(trifluoromethyl)-1H-imidazole (500 mg, 1.475 mmol, 1 equiv) in DMF (5 mL, 64.608 mmol, 43.82 equiv) were added NaH (118 mg, 4.917 mmol, 3.33 equiv) in portions at 0° C. under nitrogen atmosphere. The resulting mixture was stirred for additional 30 min at 0° C. To the above mixture was added Mel (627.89 mg, 4.425 mmol, 3 equiv) dropwise at room temperature. The resulting mixture was stirred for additional 2 h at room temperature. The reaction was quenched with Water at 0° C. The resulting mixture was extracted with EtOAc (3×10 mL). The combined organic layers were washed with brine (3×10 mL), dried over anhydrous Na₂SO₄, filtered and concentrated under reduced pressure. The residue was purified by silica gel column chromatography(eluting with PE/EA (1:1)) to afford 2-(4-bromo-2-fluoro-6-methoxyphenyl)-1-methyl-4-(trifluoromethyl)imidazole (424.4 mg, 81.51%) as a yellow oil. LC-MS-(ESI) m/z 353 [M+H]

### Step 2: Methyl 3-Fluoro-5-Methoxy-4-[1-Methyl-4-(Trifluoromethyl)Imidazol-2-Yl]Benzoate

To a stirred solution of 2-(4-bromo-2-fluoro-6-methoxyphenyl)-1-methyl-4-(trifluoromethyl)imidazole (424 mg, 1.201 mmol, 1 equiv) in MeOH (12 mL, 296.386 mmol, 246.84 equiv) were added Et 3N (364.52 mg, 3.603 mmol, 3 equiv) in portions at room temperature under carbon monoxide atmosphere. To the above mixture was added Pd(dppf)C12 (175.72 mg, mmol, 0.2 equiv) in portions at room temperature. The resulting mixture was stirred for additional 48 h at 90° C. The resulting mixture was concentrated under reduced pressure. The resulting mixture was extracted with EtOAc (3×20 mL). The combined organic layers were washed with brine (3×10 mL), dried over anhydrous Na₂SO₄, filtered and concentrated under reduced pressure. The residue was purified by silica gel column chromatography(eluting with PE/EA (3:1)) to afford methyl 3-fluoro-5-methoxy-4-[1-methyl-4-(trifluoromethyl)imidazol-2-yl]benzoate (160 mg, 40.11%) as an orange solid. LC-MS (ESI) m/z 333 [M+H]

### Step 3: {3-Fluoro-5-Methoxy-4-[1-Methyl-4-(Trifluoromethyl)Imidazol-2-Yl]Phenyl}Methanol

To a stirred solution of methyl 3-fluoro-5-methoxy-4-[1-methyl-4-(trifluoromethyl)imidazol-2-yl]benzoate (160 mg, 0.482 mmol, 1 equiv) and THF (3.2 mL, 39.497 mmol, 82.02 equiv) were added LiAlH₄ (54.83 mg, 1.446 mmol, 3 equiv) in portions at 0° C. under nitrogen atmosphere. The resulting mixture was stirred for additional 1 h at 0° C. The reaction was quenched with Water at 0° C. The resulting mixture was extracted with EtOAc (3×10 mL). The combined organic layers were washed with brine (3×10 mL), dried over anhydrous Na₂SO₄, filtered and concentrated under reduced pressure. The residue was purified by silica gel column chromatography(eluting with PE/EA (4:1)) to afford {3-fluoro-5-methoxy-4-[1-methyl-4-(trifluoromethyl)imidazol-2-yl]phenyl}methanol (120 mg, 81.90%) as an off-white solid. LC-MS (ESI) m/z 305 [M+H].

### Step 4: 2-[4-(Chloromethyl)-2-Fluoro-6-Methoxyphenyl]-1-Methyl-4-(Trifluoromethyl)Imidazole

To a stirred solution of {3-fluoro-5-m ethoxy-4-[1-methyl-4-(trifluoromethyl)imidazol-2-yl]phenyl}methanol (50 mg, 0.164 mmol, 1 equiv) and DCE (1 mL, 12.633 mmol, 76.87 equiv) were added SOCl₂ (0.04 mL, 0.492 mmol, 3 equiv) dropwise at room temperature under nitrogen atmosphere. The resulting mixture was stirred for additional 0.5 h at ° C. The reaction was quenched with Water at 0° C. The mixture was neutralized to pH 7 with saturated NaHCO₃(aq.). The resulting mixture was extracted with EtOAc (3×20 mL). The combined organic layers were washed with brine (3×10 mL), dried over anhydrous Na₂SO₄, filtered and concentrated under reduced pressure to afford 2-[4-(chloromethyl)-2-fluoro-6-methoxyphenyl]-1-methyl-4-(trifluoromethyl)imidazole (66 mg, crude) as a colorless solid. The crude product mixture was used in the next step directly without further purification. LC-MS (ESI) m/z 323 [M+H]

### Step 5: 2-(4-Cyclopropyl-6-Methoxypyrimidin-5-Yl)-8-({3-Fluoro-5-Methoxy-4-[1-Methyl-4-(Trifluoromethyl)Imidazol-2-Yl]Phenyl}Methyl)Pyrido[2,3-d]Pyrimidin-7-One

To a stirred solution of 2-[4-(chloromethyl)-2-fluoro-6-methoxyphenyl]-1-methyl-4-(trifluoromethyl)imidazole (66 mg, 0.205 mmol, 1 equiv) and DMF (1 mL, 12.922 mmol, 63.18 equiv) were added 2-(4-cyclopropyl-6-methoxypyrimidin-5-yl)-8H-pyrido[2,3-d]pyrimidin-7-one (60.40 mg, 0.205 mmol, 1 equiv) and 1,1,3,3-tetramethylguanidine (35.34 mg, 0.307 mmol, 1.5 equiv) at room temperature under nitrogen atmosphere. The resulting mixture was stirred for additional 4 h at room temperature. The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% FA) with UV detector (254 nm))2-(4-cyclopropyl-6-methoxypyrimidin-5-yl)-8-({3-fluoro-5-methoxy-4-[1-methyl-4-(trifluoromethyl)imidazo]-2-yl]phenyl methyl)pyrido[2,3-d]pyrimidin-7-one (36.3 mg, 29.79%) as an off-white solid. ¹H NMR (400 MHz, DMSO-d6) δ 9.29 (s, 1H), 8.69 (s, 1H), 8.17 (d, J=9.5 Hz, 1H), 7.96-7.91 (m, 1H), 7.04 (s, 1H), 6.91 (d, J=9.5 Hz, 1H), 6.73 (d, J=9.9 Hz, 1H), 5.56 (s, 2H), 3.82 (s, 3H), 3.70 (s, 3H), 3.43 (s, 3H), 1.75 (tt, J=8.3, 4.6 Hz, 1H), 1.06-0.99 (m, 2H), 0.83-0.74 (m, 2H). LC-MS (ESI) m/z 582[M+H]

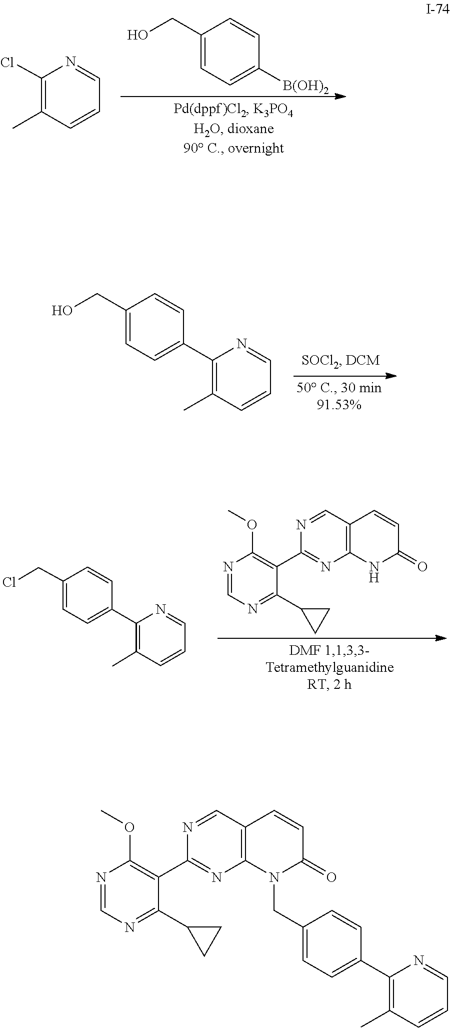

### Step 1: 14-(3-Methylpyridin-2-Yl)Phenyl]Methanol

To a stirred mixture of 2-chloro-3-methylpyridine (200 mg, 1.568 mmol, 1 equiv) and 4-(hydroxymethyl)phenylboronic acid (285.89 mg, 1.882 mmol, 1.2 equiv) in H₂O (0.4 mL) and dioxane (2 mL) was added K₃PO₄ (831.96 mg, 3.920 mmol, 2.5 equiv) and Pd(dppf)C12 (114.72 mg, 0.157 mmol, 0.1 equiv) in portions at room temperature under nitrogen atmosphere. The resulting mixture was stirred for overnight at 90° C. under nitrogen atmosphere. The mixture was allowed to cool down to room temperature. The resulting mixture was diluted with water (20 mL). The resulting mixture was extracted with EtOAc (3×20 mL). The combined organic layers were dried over anhydrous Na₂SO₄, filtered and concentrated under reduced pressure. The residue was purified by silica gel column chromatography, (eluting with PE/EA (1:7)) to afford [4-(3-methylpyridin-2-yl)phenyl]methanol (240 mg, 76.83%) as an off-white solid. LC-MS (ESI) m/z 200.2[M+H].

### Step 2: 2-[4-(Chloromethyl)Phenyl]-3-Methylpyridine

To a stirred solution of [4-(3-methylpyridin-2-yl)phenyl]methanol (100 mg, 0.502 mmol, 1 equiv) in DCE (2 mL) was added SOCl₂ (179.11 mg, 1.506 mmol, 3 equiv) dropwise at room temperature under nitrogen atmosphere. The resulting mixture was stirred for 30 min at 50° C. under nitrogen atmosphere. The mixture was allowed to cool down to room temperature. The reaction was quenched by the addition of water (20 mL) at room temperature. The mixture was neutralized to pH 7 with saturated NaHCO₃(aq.). The resulting mixture was extracted with CH₂Cl₂ (3×20 mL). The combined organic layers were dried over anhydrous Na₂SO₄, filtered and concentrated under reduced pressure to afford 2-[4-(chloromethyl)phenyl]-3-methylpyridine (100 mg, 91.53%) as a light yellow oil. LC-MS (ESI) m/z 218.1 [M+H].

### Step 3: 2-(4-Cyclopropyl-6-Methoxypyrimidin-5-Yl)-8-{14-(3-Methylpyridin-2-Yl)Phenyl]Methyl}Pyrido[2,3-d]Pyrimidin-7-One

To a stirred solution of 2-[4-(chloromethyl)phenyl]-3-methylpyridine (44 mg, mmol, 1 equiv) and 2-(4-cyclopropyl-6-methoxypyrimidin-5-yl)-8H-pyrido[2,3-d]pyrimidin-7-one (59.68 mg, 0.202 mmol, 1 equiv) in DMF (1 mL, 12.922 mmol, 63.93 equiv) was added 1,1,3,3-Tetramethylguanidine (34.92 mg, 0.303 mmol, 1.5 equiv) at room temperature under nitrogen atmosphere. The resulting mixture was stirred for 2 h at room temperature under nitrogen atmosphere. The resulting mixture was purified by reversed-phase flash chromatography(C18 silica gel column, eluting with 10% to 50% MeCN/water (0.1% FA) in 10 min with UV detector (254 nm)). The resulting mixture was concentrated under vacuum to afford 2-(4-cyclopropyl-6-methoxypyrimidin-5-yl)-8-{[4-(3-methylpyridin-2-yl)phenyl]methyl}pyrido[2,3-d]pyrimidin-7-one (34.6 mg, 35.92%) as a white solid. ¹H NMR (400 MHz, DMSO-d6) δ 9.29 (s, 1H), 8.69 (s, 1H), 8.45 (d, J=4.6 Hz, 1H), 8.16 (d, J=9.5 Hz, 1H), 7.69 (d, J=7.4 Hz, 1H), 7.46 (s, 2H), 7.34 (d, J=8.0 Hz, 2H), 7.28-7.22 (m, 1H), 6.91 (d, J=9.5 Hz, 1H), 5.57 (s, 2H), 3.83 (s, 3H), 2.28 (s, 3H), 1.72 (s, 1H), 1.01 (s, 2H), 0.76 (d, J=4.9 Hz, 2H). LC-MS (ESI) m/z 477.25 [M+H].

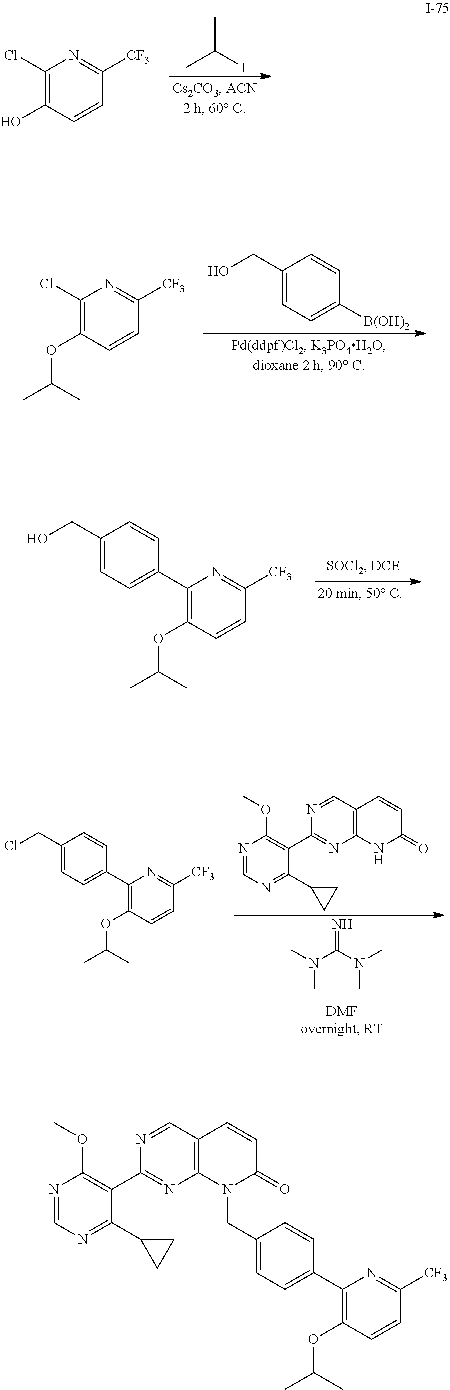

### Step 1: 2-Chloro-3-Isopropoxy-6-(Trifluoromethyl)Pyridine

A mixture of 2-chloro-6-(trifluoromethyl)pyridin-3-ol (250 mg, 1.266 mmol, 1 equiv), 2-iodopropane (322.71 mg, 1.899 mmol, 1.5 equiv) and Cs₂CO₃ (824.69 mg, 2.532 mmol, 2 equiv) in ACN (5 mL) was stirred for 2 h at 60° C. under nitrogen atmosphere. The resulting mixture was filtered, the filter cake was washed with ACN (3×5 mL). The filtrate was concentrated under reduced pressure. The residue was purified by reversed-phase flash chromatography (C18 silica gel column, eluting with 10% to 90% MeCN/water (0.1% FA) in 10 min with UV detector (254 nm)) to afford 2-chloro-3-isopropoxy-6-(trifluoromethyl)pyridine (250 mg, 82.44%) as a yellow oil. LCMS (ESI) m/z 240.0 [M+H].

### Step 2: {4-[3-Isopropoxy-6-(Trifluoromethyl)Pyridin-2-Yl]Phenyl}Methanol

A mixture of 2-chloro-3-isopropoxy-6-(trifluoromethyl)pyridine (350 mg, 1.461 mmol, 1 equiv), 4-(hydroxymethyl)phenylboronic acid (332.94 mg, 2.192 mmol, 1.5 equiv), K₃PO₄ (775.11 mg, 3.653 mmol, 2.5 equiv) and Pd(dppf)Cl₂ (213.75 mg, 0.292 mmol, 0.2 equiv) in H₂O (2 mL) and dioxane (8 mL) was stirred for 2 h at 90° C. under nitrogen atmosphere. The resulting mixture was filtered, the filter cake was washed with dioxane (3×10 mL). The filtrate was concentrated under reduced pressure. The residue was purified by silica gel column chromatography, (eluting with PE/EA (3:1)) to afford {4-[3-isopropoxy-6-(trifluoromethyl)pyridin-2-yl]phenyl}methanol (435 mg, 95.67%) as an off-white solid. LCMS (ESI) m/z 312.1 [M+H].

### Step 3: 2-[4-(Chloromethyl)Phenyl]-3-Isopropoxy-6-(Trifluoromethyl)Pyridine

A solution of {4-[3-isopropoxy-6-(trifluoromethyl)pyridin-2-yl]phenyl}methanol (435 mg, 1.397 mmol, 1 equiv) and SOCl₂ (1329.83 mg, 11.176 mmol, 8 equiv) in DCE (10 mL) was stirred for 20 min at 50° C. The resulting mixture was washed with 3×20 mL of water. The mixture was neutralized to pH 7 with saturated NaHCO₃(aq.). Then the resulting mixture was extracted with CH₂Cl₂ (3×25 mL). The combined organic layers were dried over anhydrous Na₂SO₄, filtered and the filtrate was concentrated under reduced pressure to afford 2-[4-(chloromethyl)phenyl]-3-isopropoxy-6-(trifluoromethyl)pyridine (380 mg, 82.47%) as a light yellow oil. LCMS MS (ESI) m/z 330.1 [M+H].

### Step 4: 2-(4-Cyclopropyl-6-Methoxypyrimidin-5-Yl)-8-({4-[3-Isopropoxy-6-(Trifluoromethyl)Pyridin-2-Yl]Phenyl}Methyl)Pyrido[2,3-d]Pyrimidin-7-One

A mixture of 2-[4-(chloromethyl)phenyl]-3 isopropoxy-6-(trifluoromethyl)pyri dine (50.25 mg, 0.152 mmol, 0.9 equiv), 1,1,3,3-tetramethylguanidine (29.25 mg, 0.254 mmol, 1.5 equiv) and 2-(4-cyclopropyl-6-methoxypyrimidin-5-yl)-8H-pyrido[2,3-d]pyrimidin-7-one (50 mg, 0.169 mmol, 1.00 equiv) in DMF (2 mL) was stirred for overnight at room temperature under nitrogen atmosphere. The crude product was purified by Prep-HPLC with the following conditions (Column: Sunfire prep C18 column, 30*150 mm, 51 μm; Mobile Phase A: Water(0.1% FA), Mobile Phase B: ACN; Flow rate: 60 mL/min; Gradient: 65% B to 80% B in 7 min, 80% B; Wave Length: 254/220 nm; RT1(min): 5.93;) to afford 2-(4-cyclopropyl-6-methoxypyrimidin-5-yl)-8-({4-[3 isopropoxy-6-(trifluoromethyl)pyridin-2-yl]phenyl}methyl)pyrido[2,3-d]pyrimidin-7-one (47.8 mg, 47.10%) as an off-white solid. ¹H NMR (300 MHz, DMSO-d6) δ 9.29 (s, 1H), 8.68 (s, 1H), 8.17 (d, J=9.5 Hz, 1H), 7.86-7.70 (m, 4H), 7.39-7.30 (m, 2H), 6.92 (d, J=9.5 Hz, 1H), 5.57 (s, 2H), 4.86 (hept, J=6.0 Hz, 1H), 3.82 (s, 3H), 1.70 (tt, J=8.2, 4.5 Hz, 1H), 1.30 (d, J=6.0 Hz, 6H), 0.99 (p, J=3.4 Hz, 2H), 0.75 (dt, J=8.1, 3.3 Hz, 2H). LCMS S (ESI) m/z 589.2 [M+H].

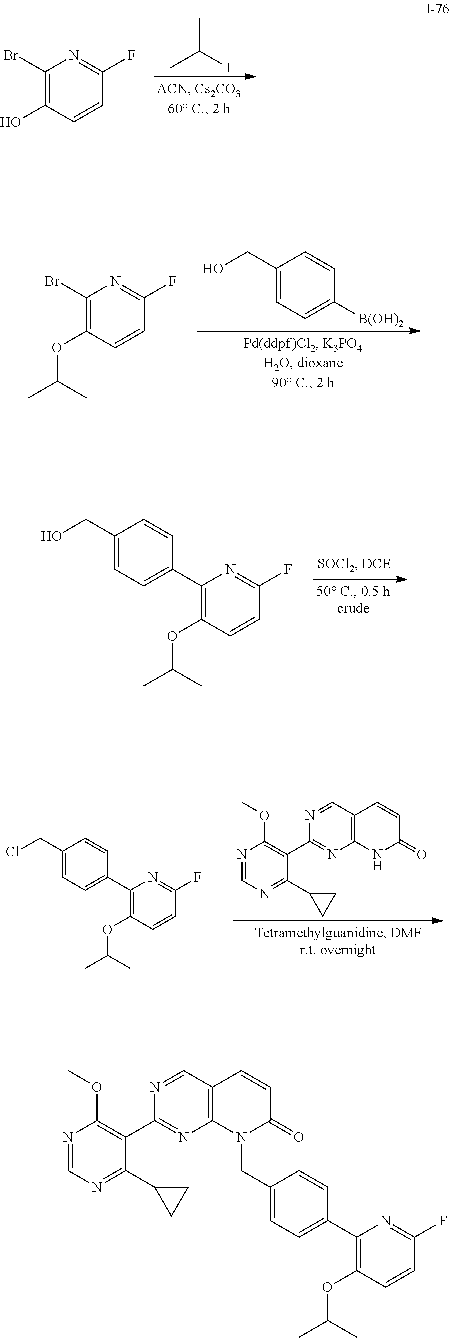

### Step1:2-Bromo-6-Fluoro-3-Isopropoxypyridine

To a stirred solution of 2-bromo-6-fluoropyridin-3-ol (200 mg, 1.042 mmol, 1 equiv) and 2-iodopropane (265.63 mg, 1.563 mmol, 1.5 equiv) in ACN (1 mL) was added Cs2CO3 (678.84 mg, 2.084 mmol, 2 equiv) at room temperature. The resulting mixture was stirred for 2 h at 60° C. under nitrogen atmosphere. The resulting mixture was filtered, the filter cake was washed with ACN (3×10 mL). The filtrate was concentrated under reduced pressure. The residue was purified by reverse flash chromatography (C18 silica gel column, eluting with 0% to 100% MeCN/water (10 mmol/L NH₄HCO₃) in 10 min with UV detector (254 nm)) to afford 2-bromo-6-fluoro-3-isopropoxypyridine (150 mg, 61.52%) as a yellow oil. LC-MS (ESI) m/z 235.0 [M+H].

### Step 2: 4-(6-Fluoro-3-Isopropoxypyridin-2-Yl)Phenyl]Methanol

To a stirred solution of 2-bromo-6-fluoro-3-isopropoxypyridine (100 mg, 0.427 mmol, 1 equiv) and 4-(hydroxymethyl)phenylboronic acid (97.38 mg, 0.640 mmol, 1.5 equiv) in dioxane (0.4 mL) and H₂O (2 mL) was added K₃PO₄ (226.71 mg, 1.067 mmol, 2.5 equiv) and Pd(dppf)Cl₂ (31.26 mg, 0.043 mmol, 0.2 equiv) at room temperature. The resulting mixture was stirred for 2 h at 90° C. under nitrogen atmosphere. The resulting mixture was filtered, the filter cake was washed with 1,4-dioxane (3×10 mL). The filtrate was concentrated under reduced pressure. The residue was purified by silica gel column chromatography, eluting with PE/EA (10:1) to afford [4-(6-fluoro-3-isopropoxypyridin-2-yl)phenyl]methanol (100 mg, 89.5%) as a yellow oil. LC-MS (ESI) m/z 262.2 [M+H].

### Step 3: 2-[4-(Chloromethyl)Phenyl]-6-Fluoro-3-Isopropoxypyridine

To a stirred solution of [4-(6-fluoro-3-isopropoxypyridin-2-yl)phenyl]methanol (100 mg, 0.383 mmol, 1 equiv) in DCE (2 mL) was added SOCl₂ (0.083 mL, 1.149 mmol, 3 equiv) dropwise at room temperature. The resulting mixture was stirred for 0.5 h at 50° C. The reaction was quenched by the addition of Water (10 mL) at room temperature. The resulting mixture was extracted with CH₂Cl₂ (3×10 mL). The combined organic layers were washed with water (3×5 mL), dried over anhydrous Na₂SO₄, filtered and concentrated under reduced pressure. The crude product was used in the next step directly without further purification. LC-MS (ESI) m/z: 280.0 [M+H]

### Step 4: 2-(4-Cyclopropyl-6-Methoxypyrimidin-5-Yl)-8-{14-(6-Fluoro-3-Isopropoxypyridin-2-Yl)Phenyl]Methyl}Pyrido[2,3-d]Pyrimidin-7-One

To a stirred solution of 2-[4-(chloromethyl)phenyl]-6-fluoro-3-isopropoxypyridine (50 mg, 0.179 mmol, 1 equiv) and 2-(4-cyclopropyl-6-methoxypyrimidin-5-yl)-8H-pyrido[2,3-d]pyrimidin-7-one (63.34 mg, 0.215 mmol, 1.2 equiv) in DMF (1 mL) was added 1,1,3,3-tetramethylguanidine (30.88 mg, 0.268 mmol, 1.5 equiv) at room temperature. The resulting mixture was stirred for overnight at room temperature under nitrogen atmosphere. The resulting mixture was extracted with EA (3×10 mL). The combined organic layers were washed with water (2×10 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-{[4-(6-fluoro-3-isopropoxypyridin-2-yl)phenyl]methyl}pyrido[2,3-d]pyrimidin-7-one (28.3 mg, 29.0%) as an off-white solid. ¹H NMR (400 MHz, DMSO-d6) δ 9.29 (s, 1H), 8.68 (s, 1H), 8.16 (d, J=9.6 Hz, 1H), 7.90-7.83 (m, 2H), 7.77 (dd, J=8.9, 6.7 Hz, 1H), 7.32 (d, J=8.3 Hz, 2H), 7.08 (dd, J=8.8, 4.0 Hz, 1H), 6.90 (d, J=9.5 Hz, 1H), 5.55 (s, 2H), 4.66 (p, J=6.0 Hz, 1H), 3.82 (s, 3H), 1.71 (tt, J=8.2, 4.6 Hz, 1H), 1.24 (d, J=6.0 Hz, 6H), 0.99 (p, J=3.5 Hz, 2H), 0.74 (m, 2H). LC-MS (ESI) m/z 539.2 [M+H].

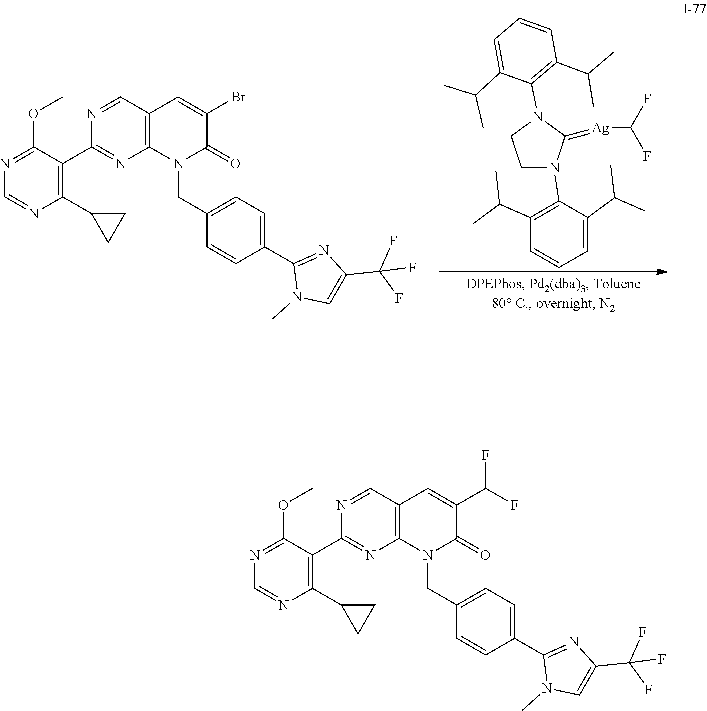

To a stirred solution of 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (30 mg, 0.049 mmol, 1 equiv) and [1,3-bis(2,6-diisopropylphenyl)imidazolidin-2-ylidene](difluoromethyl)silver (34.99 mg, 0.064 mmol, 1.3 equiv) in Toluene (1 mL) were added {242 (diphenylphosphanyl)phenoxy]phenyl}diphenylphosphane (2.64 mg, 0.005 mmol, 0.1 equiv) and Pd₂(dba)₃ (2.24 mg, 0.002 mmol, 0.05 equiv) at room temperature. The resulting mixture was stirred for overnight at 80° C. under nitrogen atmosphere. The resulting mixture was concentrated under reduced pressure. The residue was purified by Prep-HPLC with the following conditions (Column: YMC-Actus Triart C18 ExRS, 30*150 mm, 5 μm; Mobile Phase A: Water(10 mmol/L NH₄HCO₃), Mobile Phase B: ACN; Flow rate: 60 mL/min; Gradient: 37% B to 57% B in 9 min, 57% B; Wave Length: 254/220 nm; RT1(min): 10.12) to afford 2-(4-cyclopropyl-6-methoxypyrimidin-5-yl)-6-(difluoromethyl)-8-({4-[1-methyl-4-(trifluoromethyl)imidazo]-2-yl]phenyl}methyl)pyrido[2,3-d]pyrimidin-7-one (18.3 mg, 63.89%) as an off-white solid. ¹H NMR: (400 MHz, DMSO-d6) δ 9.44 (s, 1H), 8.70 (s, 1H), 8.56 (d, J=2.0 Hz, 1H), 7.91 (d, J=1.4 Hz, 1H), 7.68-7.60 (m, 2H), 7.47-7.40 (m, 2H), 7.07 (t, J=54.2 Hz, 1H), 5.59 (s, 2H), 3.84 (s, 3H), 3.74 (s, 3H), 1.76 (tt, J=8.3, 4.6 Hz, 1H), 1.03 (p, J=3.6 Hz, 2H), 0.78 (dq, J=6.8, 3.4 Hz, 2H). LC-MS (ESI) m/z 584.25 [M+H]

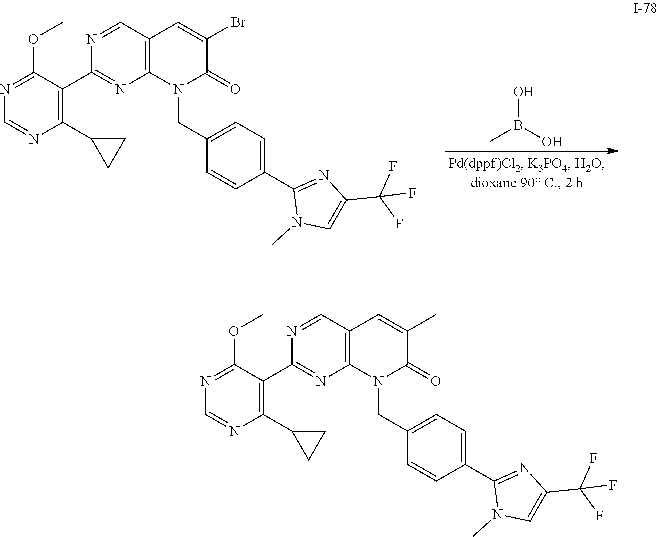

A solution of 6-bromo-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (50 mg, mmol, 1 equiv) in dioxane (2 mL) and H₂O (0.2 mg) was treated with methylboronic acid (5.86 mg, 0.098 mmol, 1.2 equiv), K₃PO₄ (43.33 mg, 0.205 mmol, 2.5 equiv) and Pd(dppf)Cl₂ (5.97 mg, 0.008 mmol, 0.1 equiv) for 2 h at 90° C. under nitrogen atmosphere. The resulting mixture was filtered, the filter cake was washed with MeOH (3×4 mL). The filtrate was concentrated under reduced pressure. The residue was purified by reverse flash chromatography (C18 silica gel column, eluting with 0% to 100% ACN/water (0.1% FA) in 15 min with UV detector (254 nm)) to afford 2-(4-cyclopropyl-6-methoxypyrimidin-5-yl)-6-methyl-8-({4-[1-methyl-4-(trifluoromethyl)imidazo]-2-yl]phenyl methyl)pyrido[2,3-d]pyrimidin-7-one (11.8 mg, 25.71%) as an off-white solid. ¹H-NMR (400 MHz, DMSO-d6): ¹H NMR (400 MHz, DMSO-d6) δ 9.21 (s, 1H), 8.68 (s, 1H), 8.02 (d, J=1.4 Hz, 1H), 7.91 (s, 1H), 7.62 (d, J=8.1 Hz, 2H), 7.40 (d, J=8.1 Hz, 2H), 5.60 (s, 2H), 3.83 (s, 3H), 3.74 (s, 3H), 2.22 (d, J=1.3 Hz, 3H), 1.71 (s, 1H), 1.01 (s, 2H), 0.80-0.72 (m, 2H). LCMS (ESI) m/z 548.25 [M+H].

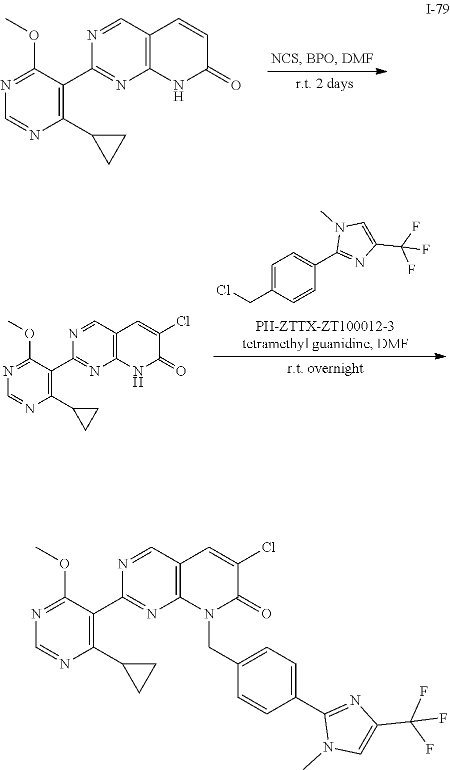 [SMILES: COc1ncnc(C2CC2)c1-c1ncc2ccc(=O)[nH]c2n1 ; Cn1cc(C(F)(F)F)nc1-c1ccc(CCl)cc1 ; COc1ncnc(C2CC2)c1-c1ncc2cc(Cl)c(=O)[nH]c2n1 ; COc1ncnc(C2CC2)c1-c1ncc2cc(Cl)c(=O)n(Cc3ccc(-c4nc(C(F)(F)F)cn4C)cc3)c2n1]

### Step 1: 6-Chloro-2-(4-Cyclopropyl-6-Methoxypyrimidin-5-Yl)-811-Pyrido[2,3-d]Pyrimidin-7-One

To a stirred solution of 2-(4-cyclopropyl-6-methoxypyrimidin-5-yl)-8H-pyrido[2,3-d]pyrimidin-7-one (50 mg, 0.169 mmol, 1 equiv) and NCS (45.22 mg, 0.338 mmol, 2 equiv) in DMF (1 mL) was added BPO (4.34 mg, 0.017 mmol, 0.1 equiv) at room temperature. The resulting mixture was stirred for 2 days at room temperature. The resulting mixture was extracted with EtOAc (3×20 mL). The combined organic layers were washed with water (2×20 mL), dried over anhydrous Na₂SO₄, filtered and was concentrated under reduced pressure. The residue was purified by reverse flash chromatography (C18 silica gel column, eluting with 0% to 100% MeCN/water (0.1% FA) in 10 min with UV detector (254 nm)) to afford 6-chloro-2-(4-cyclopropyl-6-methoxypyrimidin-5-yl)-8H-pyrido[2,3-d]pyrimidin-7-one (20 mg, 35.82%) as a yellow oil. LC-MS (ESI) m/z 330.7 [M+H].

### Step 2: 6-Chloro-2-(4-Cyclopropyl-6-Methoxypyrimidin-5-Yl)-8-({4-[1-Methyl-4-(Trifluoromethyl)Imidazol-2-Yl]Phenyl}Methyl)Pyrido[2,3-d]Pyrimidin-7-One

To a stirred mixture of 6-chloro-2-(4-cyclopropyl-6-methoxypyrimidin-5-yl)-8H-pyrido[2,3-d]pyrimidin-7-one (50 mg, 0.152 mmol, 1 equiv) and 2-[4-(chloromethyl)phenyl]-1-methyl-4-(trifluoromethyl)imidazole (41.65 mg, 0.152 mmol, 1 equiv) in DMF (1 mL) was added N,N,N′,N′-tetramethylguanidine (26.20 mg, 0.228 mmol, 1.5 equiv) at room temperature. The resulting mixture was stirred for overnight at room temperature. The residue was purified by reverse flash chromatography (C18 silica gel column, eluting with 0% to 100% MeCN/water (0.1% FA) in 10 min with UV detector (254 nm)) to afford 6-chloro-2-(4-cyclopropyl-6-methoxypyrimidin-5-yl)-8-({4-[1-methyl-4-(trifluoromethyl)imidazo]-2-yl]phenyl}methyl)pyrido[2,3-d]pyrimidin-7-one (22.2 mg, 25.62%) as an off-white solid. ¹H NMR (400 MHz, DMSO-d6) δ 9.29 (s, 1H), 8.70 (s, 1H), 8.57 (s, 1H), 7.92 (d, J=1.4 Hz, 1H), 7.67-7.60 (m, 2H), 7.43 (d, J=8.1 Hz, 2H), 5.63 (s, 2H), 3.83 (s, 3H), 3.74 (s, 3H), 1.73 (tt, J=8.1, 4.6 Hz, 1H), 1.02 (m, 2H), 0.88-0.73 (m, 2H). LC-MS (ESI) m/z 568.1 [M+H].

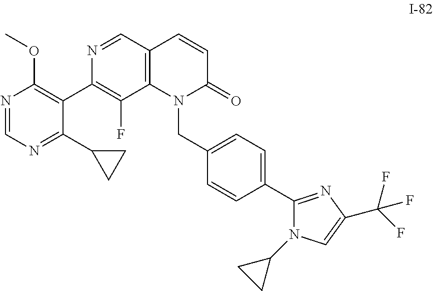 [SMILES: COc1ncnc(C2CC2)c1-c1ncc2ccc(=O)n(Cc3ccc(-c4nc(C(F)(F)F)cn4C4CC4)cc3)c2c1F]

1-82 was made in an analogous way as 1-27.

¹H NMR (400 MHz, DMSO-d6) δ 8.92 (s, 1H), 8.67 (s, 1H), 8.23 (dd, J=9.6, 1.9 Hz, 1H), 7.91 (d, J=1.4 Hz, 1H), 7.87-7.80 (m, 2H), 7.25 (d, J=8.1 Hz, 2H), 6.97 (d, J=9.6 Hz, 1H), 5.65 (s, 2H), 3.73 (s, 3H), 3.68 (m, 1H), 1.60 (m, 1H), 1.06 (d, J=6.1 Hz, 1H), 1.01-(m, 6H), 0.72 (s, 1H). LC-MS (ESI) m/z 577.2 [M+H].

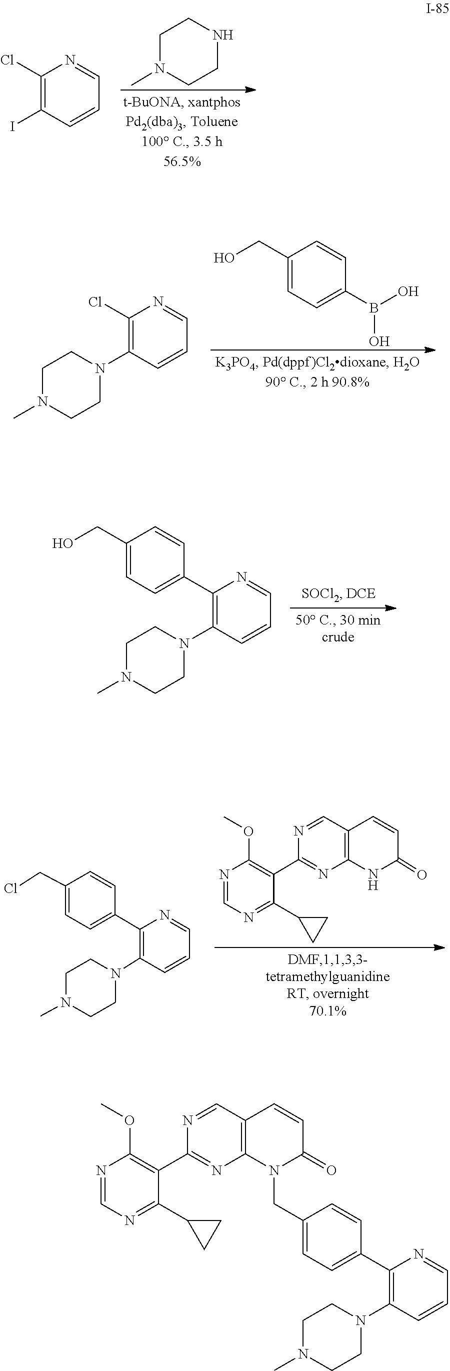

### Step 1: 1-(2-Chloropyridin-3-Yl)-4-Methylpiperazine

To a stirred mixture of 2-chloro-3-iodopyridine (500 mg, 2.088 mmol, 1 equiv) and piperazine, 1-methyl-(158.9 mg, 1.587 mmol, 0.76 equiv) in Toluene (10 mL) were added t-BuONa (230.8 mg, 2.401 mmol, 1.15 equiv), xantphos (54.4 mg, 0.094 mmol, 0.045 equiv) and Pd₂(dba)₃ (28.7 mg, 0.031 mmol, 0.015 equiv) at room temperature under nitrogen atmosphere. The resulting mixture was stirred for 3.5h at 100° C. under nitrogen atmosphere. The mixture was allowed to cool down to room temperature. The resulting mixture was diluted with water (50 mL). The resulting mixture was extracted with EtOAc (3×50 mL). The combined organic layers were washed with brine (3×50 mL), dried over anhydrous Na₂SO₄, filtered and concentrated under reduced pressure. The residue was purified by reverse flash chromatography (C18 silica gel column, eluting with 0% to 100% MeCN/water (10 mmol/L NH₄HCO₃) in 10 min with UV detector (254 nm)) to afford 1-(2-chloropyridin-3-yl)-4-methylpiperazine (250 mg, 56.55%) as a yellow oil.

LC-MS (ESI) m/z 212.1 [M+H]

### Step 2: {4-[3-(4-Methylpiperazin-1-Yl)Pyridin-2-Yl]Phenyl}Methanol

To a stirred mixture of 1-(2-chloropyridin-3-yl)-4-methylpiperazine (370 mg, 1.748 mmol, 1 equiv) and 4-(hydroxymethyl)phenylboronic acid (398.4 mg, 2.622 mmol, 1.5 equiv) in dioxane (6.00 mL, 70.829 mmol, 40.52 equiv) and H₂O (1.50 mL, 83.275 mmol, 47.64 equiv) were added K₃PO₄ (927.5 mg, 4.370 mmol, 2.5 equiv) and Pd(dppf)C12 (255.8 mg, 0.350 mmol, 0.2 equiv) at room temperature under nitrogen atmosphere. The resulting mixture was stirred for 2 h at 90° C. under nitrogen atmosphere. The mixture was allowed to cool down to room temperature. The resulting mixture was diluted with water (30 mL). The resulting mixture was extracted with EtOAc (3×20 mL). The combined organic layers were washed with brine (3×mL), dried over anhydrous Na₂SO₄, filtered and concentrated under reduced pressure. The residue was purified by reverse flash chromatography (C18 silica gel column, eluting with 0% to 100% MeCN/water (10 mmol/L NH₄HCO₃) in 10 min with UV detector (254 nm)) to afford {4-[3-(4-methylpiperazin-1-yl)pyri din-2-yl]phenyl}methanol (450 mg, 90.86%) as an off-white solid.

LC-MS (ESI) m/z 284.2 [M+H]

### Step 3: 1-{2-[4-(Chloromethyl)Phenyl]Pyridin-3-Yl}-4-Methylpiperazine

To a stirred mixture of {4-[3-(4-methylpiperazin-1-yl)pyridin-2-yl]phenyl}methanol (100 mg, 0.353 mmol, 1 equiv) in DCE (2 mL, 25.265 mmol, 71.60 equiv) was added thionyl chloride (125.9 mg, 1.059 mmol, 3 equiv) at room temperature under nitrogen atmosphere. The resulting mixture was stirred for 30 min at under nitrogen atmosphere. The mixture was allowed to cool down to room temperature. The resulting mixture was diluted with water (30 mL). The resulting mixture was extracted with EtOAc (3×30 mL). The combined organic layers were washed with brine (3×20 mL), dried over anhydrous Na₂SO₄, filtered and concentrated under reduced pressure to afford 1-{2-[4-(chloromethyl)phenyl]pyridin-3-yl}-4-methylpiperazine (105 mg, crude) as a brown solid.

LC-MS (ESI) m/z 302.1 [M+H]

### Step 4: 2-(4-Cyclopropyl-6-Methoxypyrimidin-5-Yl)-8-({4-[3-(4-Methylpiperazin-1-Yl)Pyridin-2-Yl]Phenyl}Methyl)Pyrido[2,3-d]Pyrimidin-7-One

To a stirred mixture of 1-{2-[4-(chloromethyl)phenyl]pyri din-3-yl}-4-methylpiperazine (50 mg, 0.166 mmol, 1 equiv) and 2-(4-cyclopropyl-6-methoxypyrimidin-5-yl)-8H-pyrido[2,3-d]pyrimidin-7-one (48.9 mg, 0.166 mmol, 1 equiv) in dimethylformamide (1 mL, mmol) was added 1,1,3,3-tetramethylguanidine (47.70 mg, 0.415 mmol, 2.5 equiv) at room temperature under nitrogen atmosphere. The resulting mixture was stirred for overnight at room temperature under nitrogen atmosphere. The resulting mixture was purified by Prep-HPLC with the following conditions (Column: YMC-Actus Triart C18 ExRS, 30*150 mm, 51 μm; Mobile Phase A: Water(10 mmol/L NH₄HCO₃), Mobile Phase B: MeOH-HPLC; Flow rate: 60 mL/min; Gradient: 53% B to 70% B in 9 min, 70% B; Wave Length: 254/220 nm; RT1(min): 10.70) to afford 2-(4-cyclopropyl-6-methoxypyrimidin-5-yl)-8-({4-[3-(4-methylpiperazin-1-yl)pyridin-2-yl]phenyl}methyl)pyrido[2,3-d]pyrimidin-7-one (66 mg, 70.14%) as an off-white solid.

LC-MS (ESI) m/z 561.1 [M+H]

¹H NMR (400 MHz, DMSO-d6) δ 9.29 (s, 1H), 8.69 (s, 1H), 8.28-8.11 (m, 2H), 7.87 (d, J=8.2 Hz, 2H), 7.45 (dd, J=8.2, 1.5 Hz, 1H), 7.33 (d, J=8.3 Hz, 2H), 7.25 (dd, J=8.1, 4.6 Hz, 1H), 6.90 (d, J=9.5 Hz, 1H), 5.55 (s, 2H), 3.84 (s, 3H), 2.76 (m, 4H), 2.27 (s, 4H), 2.14 (s, 3H), 1.73 (m, J=8.0, 4.5, 4.1 Hz, 1H), 1.01 (dd, J=3.4 Hz, 2H), 0.79 (dd, J=7.9, 3.4 Hz, 2H).

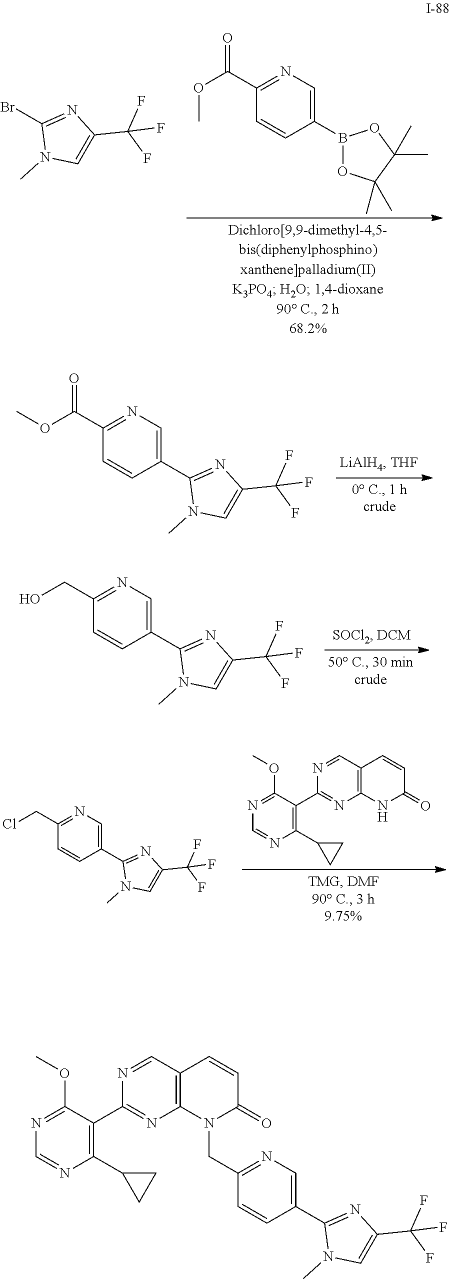

### Step1: Methyl 5-[1-Methyl-4-(Trifluoromethyl)Imidazol-2-Yl]Pyridine-2-Carboxylate

To a stirred mixture of 2-bromo-1-methyl-4-(trifluoromethyl)imidazole (200 mg, mmol, 1 equiv) and methyl 5-(4,4,5,5-tetramethyl-1,3,2-dioxaborolan-2-yl)pyri dine-2-carboxylate (344.6 mg, 1.309 mmol, 1.5 equiv) in H₂O (0.8 mL, 13.968 mmol, 4 equiv) and 1,4-dioxane (3.2 mL, 3.492 mmol, 16 equiv) were added [5-(diphenylphosphanyl)-9,9-dimethyl-9H-xanthen-4-yl]diphenylphosphane; dichloropalladium (132.0 mg, 0.175 mmol, 0.2 equiv) and K₃PO₄ (556.1 mg, 2.619 mmol, 3.0 equiv) at room temperature under nitrogen atmosphere. The resulting mixture was stirred for 2 h at 90 C under nitrogen atmosphere. The mixture was allowed to cool down to room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reverse flash chromatography (C18 silica gel column, eluting with 0% to 100% MeCN/water (0.1% FA) in 10 min with UV detector (254 nm)) to afford methyl 5-[1-methyl-4-(trifluoromethyl)imidazo]-2-yl]pyridine-2-carboxylate (170 mg, 68.24%) as a brown oil.

LC-MS (ESI) m/z 286.1 [M+H]

### Step 2: {5-[1-Methyl-4-(Trifluoromethyl)Imidazol-2-Yl]Pyridin-2-Yl}Methanol

To a stirred methyl 5-[1-methyl-4-(trifluoromethyl)imidazo]-2-yl]pyridine-2-carboxylate (200 mg, 0.701 mmol, 1 equiv) in THF (4 mL, 20 equiv) was added LiAlH₄ (79.8 mg, 2.103 mmol, 3.0 equiv) at 0° C. under nitrogen atmosphere. The resulting mixture was stirred for 1 h at 0° C. under nitrogen atmosphere. The resulting mixture was filtered, the filter cake was washed with THF (3×5 mL). The filtrate was concentrated under reduced pressure to afford{5-[1-methyl-4-(trifluoromethyl)imidazo]-2-yl]pyridin-2-yl}methanol (165 mg, crude) as a light yellow oil. The crude resulting mixture was used in the next step directly without further purification.

LC-MS (ESI) m/z 258.2 [M+H]

### Step 3: 2-(Chloromethyl)-5-[1-Methyl-4-(Trifluoromethyl)Imidazol-2-Yl]Pyridine

To a stirred {5-[1-methyl-4-(trifluoromethyl)imidazo]-2-yl]pyridin-2-yl}methanol (166 mg, 0.645 mmol, 1 equiv) in DCM (3.3 mL, 20 equiv) was added SOCl₂ (230.3 mg, 1.935 mmol, 3.0 equiv) dropwise at room temperature under nitrogen atmosphere. The resulting mixture was stirred for 30 min at 50° C. under nitrogen atmosphere. The reaction was quenched with Water at 0° C. The residue was neutralized to pH 7 with saturated NaHCO₃(aq.). The resulting mixture was extracted with EtOAc (3×2 mL). The combined organic layers were washed with brine (2×5 mL), dried over anhydrous Na₂SO₄, filtered and concentrated under reduced pressure to afford 2-(chloromethyl)-5-[1-methyl-4-(trifluoromethyl)imidazo]-2-yl]pyridine (130 mg, crude) as a brown oil. The crude resulting mixture was used in the next step directly without further purification.

LC-MS (ESI) m/z 276.1 [M+H]

### Step 4: 2-(4-Cyclopropyl-6-Methoxypyrimidin-5-Yl)-8-({5-[1-Methyl-4-(Trifluoromethyl)Imidazol-2-Yl]Pyridin-2-Yl}Methyl)Pyrido[2,3-d]Pyrimidin-7-One

To a stirred mixture of 2-(chloromethyl)-5-[1-methyl-4-(trifluoromethyl)imidazol-2-yl]pyridine (50 mg, 0.181 mmol, 1 equiv) and 2-(4-cyclopropyl-6-methoxypyrimidin-5-yl)-8H-pyrido[2,3-d]pyrimidin-7-one (53.5 mg, 0.181 mmol, 1 equiv) in DMF (1 mL) was added N,N,N′,N′-tetramethylguanidine (31.3 mg, 0.271 mmol, 1.5 equiv) at room temperature under nitrogen atmosphere. The resulting mixture was stirred for 3h at room temperature under nitrogen atmosphere. The resulting mixture was purified by Prep-HPLC with the following conditions (Column: Xselect CSH F-Phenyl OBD column, 19*250 mm, 5 μm; Mobile Phase A: Water(0.1% FA), Mobile Phase B: ACN; Flow rate: 25 mL/min; Gradient: 35% B to 50% B in 8 min; Wave Length: 254/220 nm; RT1(min): 7.40) to afford 2-(4-cyclopropyl-6-methoxypyrimidin-5-yl)-8-({5-[1-methyl-4-(trifluoromethyl)imidazo]-2-yl]pyridin-2-yl}methyl)pyrido[2,3-d]pyrimidin-7-one (9.5 mg, 9.75%) as a light yellow solid.

LC-MS (ESI) m/z 535.2[M+H]

¹H NMR (400 MHz, DMSO-d6) δ 9.29 (s, 1H), 8.69 (d, J=2.3 Hz, 1H), 8.64 (s, 1H), 8.19 (d, J=9.5 Hz, 1H), 8.08 (dd, J=8.2, 2.3 Hz, 1H), 7.98 (s, 1H), 7.45 (d, J=8.2 Hz, 1H), 6.92 (d, J=9.5 Hz, 1H), 5.71 (s, 2H), 3.75 (d, J=4.5 Hz, 6H), 1.62 (tt, J=8.2, 4.6 Hz, 1H), 0.92 (p, J=3.7 Hz, 2H), 0.62 (dq, J=7.0, 3.5 Hz, 2H).

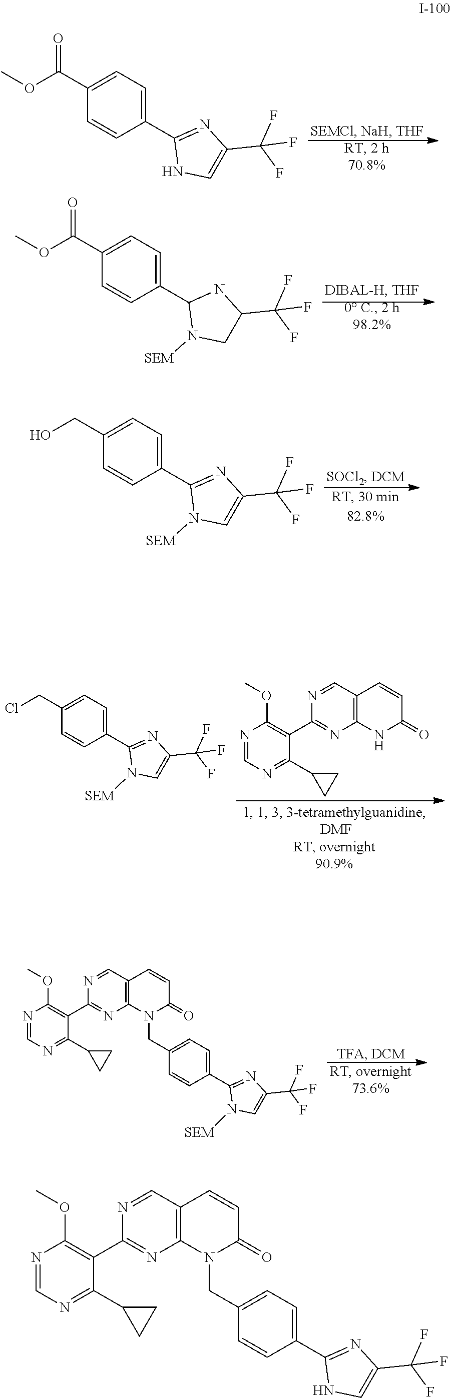

### Step 1: Methyl 4-[4-(Trifluoromethyl)-1-{12-(Trimethylsilyl)Ethoxy]Methyl}Imidazol-2-Yl]Benzoate

A solution of methyl 4-[4-(trifluoromethyl)-1H-imidazol-2-yl]benzoate (2 g, 7.402 mmol, 1 equiv) in THF (40 mL, 493.711 mmol, 66.70 equiv) was treated with NaH (355.2 mg, 14.804 mmol, 2 equiv) for 30 min at 0° C. under nitrogen atmosphere followed by the addition of SEMCl (1.851 g, 11.103 mmol, 1.5 equiv) dropwise at 0° C. The resulting mixture was stirred for 2 h at room temperature under nitrogen atmosphere. The reaction was quenched by the addition of Water/Ice (100 mL) at 0° C. The resulting mixture was extracted with EtOAc (3×100 mL). The combined organic layers were washed with brine (3×100 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford methyl 4-[4-(trifluoromethyl)-1-{[2-(trimethylsilyl)ethoxy]methyl}imidazol-2-yl]benzoate (2.1 g, 70.85%) as a yellow oil.

LC-MS (ESI) m/z 401.1 [M+H]

### Step 2: {4-[4-(Trifluoromethyl)-1-{12-(Trimethylsilyl)Ethoxy]Methyl}Imidazol-2-Yl]Phenyl}Methanol

To a stirred mixture of methyl 4-[4-(trifluoromethyl)-1-{[2-(trimethylsilyl)ethoxy]methyl}imidazol-2-yl]benzoate (3 g, 7.491 mmol, 1 equiv) in THF (60 mL, 740.566 mmol, 98.86 equiv) was added DIBAL-H (29.96 mL, 29.964 mmol, 4 equiv) dropwise at 0° C. under nitrogen atmosphere. The resulting mixture was stirred for 2 h at under nitrogen atmosphere. The reaction was quenched by the addition of Water/Ice (100 mL) at 0° C. The resulting mixture was extracted with EtOAc (3×100 mL). The combined organic layers were washed with brine (3×100 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford {444-(trifluoromethyl)-1-{[2-(trimethylsilyl)ethoxy]methyl}imidazol-2-yl]phenyl}methanol (2.74 g, 98.20%) as a yellow oil.

LC-MS (ESI) m/z 373.1 [M+H]

### Step3: 2-[4-(Chloromethyl)Phenyl]-4-(Trifluoromethyl)-1-{12-(Trimethylsilyl)Ethoxy]Methyl}Imidazole

To a stirred mixture of {4-[4-(trifluoromethyl)-1-{[2-(trimethyl silyl)ethoxy]methyl imidazol-2-yl]phenyl}methanol (1 g, 2.685 mmol, 1 equiv) in DCM (20 mL, 314.612 mmol, 117.18 equiv) was added thionyl chloride (0.80 g, 6.713 mmol, 2.5 equiv) dropwise at 0° C. under nitrogen atmosphere. The resulting mixture was stirred for 30 min at room temperature under nitrogen atmosphere. The reaction was quenched by the addition of Water/Ice (100 mL) at 0° C. The resulting mixture was extracted with EtOAc (3×100 mL). The combined organic layers were washed with brine (3×100 mL), dried over anhydrous Na₂SO₄, filtered and concentrated under reduced pressure. The residue was purified by silica gel column chromatography, (eluting with PE/EA (9:1)) to afford 2-[4-(chloromethyl)phenyl]-4-(trifluoromethyl)-1-{[2-(trimethylsilyl)ethoxy]methyl imidazole (870 mg, 82.89%) as a yellow oil.

LC-MS (ESI) m/z 391.1 [M+H]

### Step 4: 2-(4-Cyclopropyl-6-Methoxypyrimidin-5-Yl)-8-({4-[4-(Trifluoromethyl)-1-{12-(Trimethylsilyl)Ethoxy]Methyl}Imidazol-2-Yl]Phenyl}Methyl)Pyrido[2,3-d]Pyrimidin-7-One

To a stirred mixture of 2-[4-(chloromethyl)phenyl]-4-(trifluoromethyl)-1-{[2-(trimethylsilyl)ethoxy]methyl}imidazole (661.9 mg, 1.693 mmol, 1 equiv) and 2-(4-cyclopropyl-6-methoxypyrimidin-5-yl)-8H-pyrido[2,3-d]pyrimidin-7-one (500 mg, 1.693 mmol, 1.00 equiv) in DMF (10 mL, 129.215 mmol, 76.32 equiv) was added 1,1,3,3-tetramethylguanidine (292.5 mg, 2.540 mmol, 1.5 equiv) dropwise at room temperature under nitrogen atmosphere. The resulting mixture was stirred for overnight at room temperature under nitrogen atmosphere. The resulting mixture was diluted with water (50 mL). The resulting mixture was extracted with EtOAc (3×50 mL). The combined organic layers were washed with brine (3×50 mL), dried over anhydrous Na₂SO₄, filtered and concentrated under reduced pressure. The residue was purified by silica gel column chromatography, (eluting with PE/EA (1:1)) to afford 2-(4-cyclopropyl-6-methoxypyrimidin-5-yl)-8-({4-[4-(trifluoromethyl)-1-{[2-(trimethyl silyl)ethoxy]methyl imidazol-2-yl]phenyl methyl)pyri do[2,3-d]pyrimidin-7-one (1 g, as a colorless oil.

LC-MS (ESI) m/z 650.2 [M+H]

### Step 5: 2-(4-Cyclopropyl-6-Methoxypyrimidin-5-Yl)-8-({4-[4-(Trifluoromethyl)-1H-Imidazol-2-Yl]Phenyl}Methyl)Pyrido[2,3-d]Pyrimidin-7-One

A mixture of 2-(4-cyclopropyl-6-methoxypyrimidin-5-yl)-8-({4-[4-(trifluoromethyl)-1-{[2-(trimethylsilyl)ethoxy]methyl imidazol-2-yl]phenyl methyl)pyrido[2,3-d]pyrimidin-7-one (500 mg, 0.770 mmol, 1 equiv) in TFA(5 mL) and DCM (5 mL, 78.653 mmol, 102.21 equiv) was stirred for overnight at room temperature under nitrogen atmosphere. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-({4-[4-(trifluoromethyl)-1H-imidazol-2-yl]phenyl methyl)pyrido[2,3-d]pyrimidin-7-one (300 mg, 73.62%) as an off-white solid.

LC-MS (ESI) m/z 520.2 [M+H]

¹H NMR (400 MHz, DMSO-d6) δ 13.15 (s, 1H), 9.28 (s, J=0.9 Hz, 1H), 8.69 (s, 1H), 8.15 (d, J=9.6, 0.8 Hz, 1H), 7.95-7.83 (m, 3H), 7.39 (d, J=8.1 Hz, 2H), 6.90 (d, J=9.5 Hz, 1H), 5.54 (s, 2H), 3.83 (s, 3H), 1.72 (m, J=8.2, 4.6 Hz, 1H), 1.02 (m, J=3.7 Hz, 2H), 0.76 (m, J=7.1, 3.6 Hz, 2H).

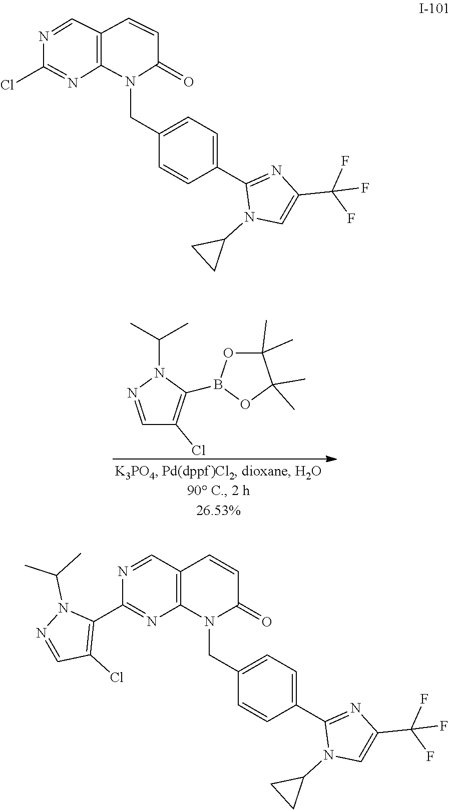 [SMILES: O=c1ccc2cnc(Cl)nc2n1Cc1ccc(-c2nc(C(F)(F)F)cn2C2CC2)cc1 ; CC(C)n1ncc(Cl)c1B1OC(C)(C)C(C)(C)O1 ; CC(C)n1ncc(Cl)c1-c1ncc2ccc(=O)n(Cc3ccc(-c4nc(C(F)(F)F)cn4C4CC4)cc3)c2n1]

To a stirred mixture of 2-chloro-8-({4-[1-cyclopropyl-4-(trifluoromethyl)imidazo]-2-yl]phenyl}methyl)pyrido[2,3-d]pyrimidin-7-one (30 mg, 0.067 mmol, 1 equiv) and 4-chloro-1-isopropyl-5-(4,4,5,5-tetramethyl-1,3,2-dioxaborolan-2-yl)pyrazole (21.9 mg, 0.080 mmol, 1.2 equiv) in dioxane (1 mL, 11.804 mmol, 175.42 equiv) and H₂O (0.8 mL, 44.407 mmol, 659.94 equiv) was added K₃PO₄ (42.9 mg, 0.201 mmol, 3 equiv) and Pd(dppf)C12 (9.9 mg, 0.013 mmol, equiv) at room temperature under nitrogen atmosphere. The resulting mixture was stirred for additional 2 h at 90° C. The mixture was allowed to cool down to room temperature. The resulting mixture was filtered, the filter cake was washed with 1,4-dioxane (2×2 mL). The filtrate was concentrated under reduced pressure. The residue was purified by reversed-phase flash chromatography(C18 silica gel column, eluting with 0% to 100% MeCN/water (0.1% FA) in 10 min with UV detector (254 nm)).to afford 2-(4-chloro-2-isopropylpyrazol-3-yl)-8-({4-[1-cyclopropyl-4-(trifluoromethyl)imidazol-2-yl]phenyl methyl)pyrido[2,3-d]pyrimidin-7-one (10 mg, 26.53%) as a white solid.

LC-MS (ESI) m/z 554.0 [M+H]

¹H NMR (400 MHz, DMSO-d6) δ 9.33 (s, 1H), 8.18 (d, J=9.5 Hz, 1H), 7.90 (d, J=1.4 Hz, 1H), 7.85-7.78 (m, 2H), 7.74 (s, 1H), 7.33 (d, J=8.1 Hz, 2H), 6.93 (d, J=9.5 Hz, 1H), (s, 2H), 5.26 (h, J=6.6 Hz, 1H), 3.69 (t, J=5.2 Hz, 1H), 1.26 (d, J=6.6 Hz, 6H), 0.96 (q, J=6.7, 6.2 Hz, 2H), 0.89 (dd, J=6.0, 3.2 Hz, 2H).

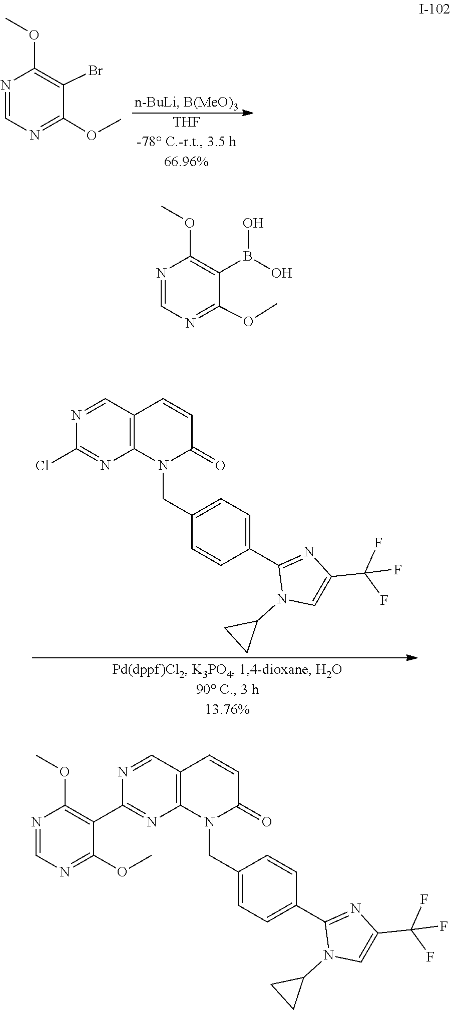 [SMILES: COc1ncnc(OC)c1Br ; COc1ncnc(OC)c1B(O)O ; O=c1ccc2cnc(Cl)nc2n1Cc1ccc(-c2nc(C(F)(F)F)cn2C2CC2)cc1 ; COc1ncnc(OC)c1-c1ncc2ccc(=O)n(Cc3ccc(-c4nc(C(F)(F)F)cn4C4CC4)cc3)c2n1]

### Step 1: 4,6-Dimethoxypyrimidin-5-Ylboronic Acid

To a stirred solution of 5-bromo-4,6-dimethoxypyrimidine (500 mg, 2.283 mmol, 1 equiv) in tetrahydrofuran (10 mL) was added n-BuLi (1.37 mL, 3.425 mmol, 1.5 equiv, 2.5M in hexane) dropwise at −78° C. under nitrogen atmosphere. The resulting mixture was stirred for additional 0.5 h at −78° C. To the above mixture was added Trimethyl borate (355.8 mg, 3.425 mmol, 1.5 equiv) dropwise at −78° C. The resulting mixture was stirred for additional 3 h at room temperature. The mixture was acidified to pH=3 with HCl (aq.). The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% FA) with UV detector (254 nm)) to yield 4,6-dimethoxypyrimidin-5-ylboronic acid (281.2 mg, 66.96%) as an off-white solid. LC-MS (ESI) m/z 185 [M+H]

### Step 2: 8-({4-[1-Cyclopropyl-4-(Trifluoromethyl)Imidazol-2-Yl]Phenyl}Methyl)-2-(4,6-Dimethoxypyrimidin-5-Yl)Pyrido[2,3-d]Pyrimidin-7-One

To a stirred solution of 4,6-dimethoxypyrimidin-5-ylboronic acid (18.5 mg, 0.101 mmol, 1.5 equiv) and 2-chloro-8-({4-[1-cyclopropyl-4-(trifluoromethyl)imidazol-2-yl]phenyl methyl)pyrido[2,3-d]pyrimidin-7-one (30 mg, 0.067 mmol, 1.00 equiv) in 1,4-dioxane (0.6 mL, 7.082 mmol, 105.25 equiv) were added K₃PO₄ (42.85 mg, 0.201 mmol, 3 equiv) and H₂O (0.1 mL, 5.551 mmol, 82.49 equiv) at room temperature under nitrogen atmosphere. To the above mixture was added Pd(dppf)C12 (9.8 mg, 0.013 mmol, 0.2 equiv) at room temperature. The resulting mixture was stirred for additional 3 h at 90° C. The resulting mixture was diluted with H₂O (10 mL). The resulting mixture was extracted with EtOAc (3×10 mL). The combined organic layers were washed with brine (3×10 mL), dried over anhydrous Na₂SO₄, filtered and concentrated under reduced pressure. The residue was purified by reverse flash chromatography

(C18 silica gel column, eluting with 0% to 100% in 10 min MeCN/water (0.1% TFA) with UV detector (254 nm)) to yield 8-({4-[1-cyclopropyl-4-(trifluoromethyl)imidazo]-2-yl]phenyl methyl)-2-(4,6-dimethoxypyrimidin-5-yl)pyrido[2,3-d]pyrimidin-7-one (5.1 mg, 13.76%) as an off-white solid

LC-MS (ESI) m/z 550 [M+H]

¹H NMR (400 MHz, DMSO-d6) δ 9.23 (s, 1H), 8.63 (s, 1H), 8.12 (d, J=9.5 Hz, 1H), 7.91 (d, J=1.4 Hz, 1H), 7.86-7.79 (m, 2H), 7.45 (d, J=8.2 Hz, 2H), 6.88 (d, J=9.5 Hz, 1H), 5.53 (s, 2H), 3.85 (s, 6H), 3.70 (tt, J=7.1, 3.8 Hz, 1H), 1.01-0.91 (m, 2H), 0.95-0.83 (m, 2H).

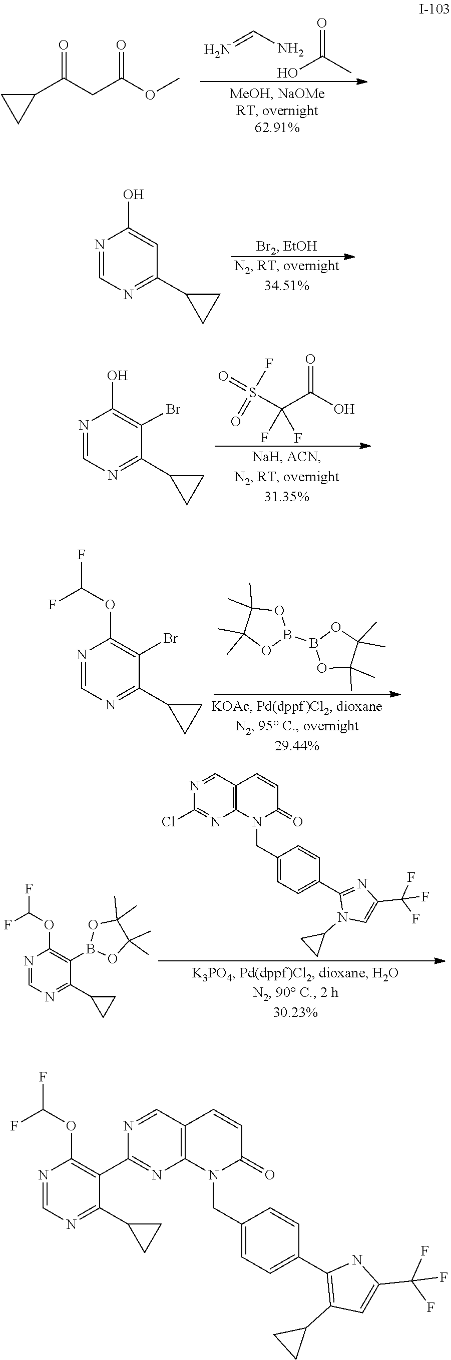

### Step 1: 6-Cyclopropylpyrimidin-4-Ol

To a stirred solution of methyl 3-cyclopropyl-3-oxopropanoate (5 g, 26.851 mmol, 1 equiv) and Formamidine acetate (7.32 g, 70.346 mmol, 2 equiv) in MeOH (100 mL) was added NaOMe (10.15 g, 187.957 mmol, 7 equiv) at room temperature. The resulting mixture was stirred for overnight at room temperature. The resulting mixture was concentrated under reduced pressure. The reaction was quenched with Water (50 mL) at room temperature. The resulting mixture was extracted with EtOAc (2×30 mL). The aqueous layer was acidified to pH 6 with HCl (aq.) and extracted with EtOAc (3×20 mL), dried over anhydrous sodium sulfate and concentrated under reduced pressure to afford 6-cyclopropylpyrimidin-4-ol (2.3 g, 62.91%) as a yellow green crude solid.

LC-MS (ESI) m/z 137.1 [M+H]

### Step 2: 5-Bromo-6-Cyclopropylpyrimidin-4-Ol

To a stirred solution of 6-cyclopropylpyrimidin-4-ol (2 g, 14.689 mmol, 1 equiv) in EtOH (20 mL) was added Br2 (2.26 mL, 44.067 mmol, 3 equiv) dropwise at 0° C. under nitrogen atmosphere. The resulting mixture was stirred for overnight at room temperature under nitrogen atmosphere. The resulting mixture was concentrated under reduced pressure. The reaction was quenched by the addition of sat. Na₂S₂O₃ (aq.) (10 mL) at room temperature. The resulting mixture was extracted with CH₂Cl₂ (3×20 mL). The combined organic layers were dried over anhydrous Na₂SO₄ and concentrated under reduced pressure. The residue was purified by silica gel column chromatography, (eluting with CH₂Cl₂/MeOH (10:1)) to afford 5-bromo-6-cyclopropylpyrimidin-4-ol (1.09 g, 34.51%) as a yellow oil.

LC-MS (ESI) m/z 216.0 [M+H]

### Step 3: 5-Bromo-4-Cyclopropyl-6-(Difluoromethoxy)Pyrimidine

To a stirred solution of 5-bromo-6-cyclopropylpyrimidin-4-ol (740 mg, 3.441 mmol, 1 equiv) in ACN (5 mL) was added NaH (412.9 mg, 10.323 mmol, 3 equiv, 60% in minreal oil) at room temperature under nitrogen atmosphere. The resulting mixture was stirred for 20 min at room temperature under nitrogen atmosphere. To the above mixture was added difluoro(sulfo)acetic acid (1.23 g, 6.882 mmol, 2 equiv) at room temperature. The resulting mixture was stirred for additional overnight at room temperature. The reaction was quenched with sat. NH₄Cl (aq.) at room temperature. The aqueous layer was extracted with EtOAc (3×15 mL). The combined organic layers were dried over anhydrous Na₂SO₄, filtered and concentrated under reduced pressure. The residue was purified by reversed-phase flash chromatography (C18 silica gel column, eluting with 0% to 100% MeCN/water (0.1% FA) in 20 min with UV detector (254 nm)) to afford 5-bromo-4-cyclopropyl-6-(difluoromethoxy)pyrimidine (285.9 mg, 31.35%) as a brown oil.

LC-MS (ESI) m/z 265.0 [M+H]

### Step 4: 4-Cyclopropyl-6-(Difluoromethoxy)-5-(4,4,5,5-Tetramethyl-1,3,2-Dioxaborolan-2-Yl)Pyrimidine

To a stirred solution of 5-bromo-4-cyclopropyl-6-(difluoromethoxy)pyrimidine (225 mg, 0.849 mmol, 1 equiv) and bis(pinacolato)diboron (431.2 mg, 1.698 mmol, 2 equiv) in dioxane (3 mL) were added KOAc (249.93 mg, 2.547 mmol, 3 equiv) and Pd(dppf)C12 (93.2 mg, mmol, 0.15 equiv) at room temperature. The resulting mixture was stirred for 2 h at 95° C. under nitrogen atmosphere. The resulting mixture was filtered, the filter cake was washed with THF (2×3 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford 4-cyclopropyl-6-(difluoromethoxy)-5-(4,4,5,5-tetramethyl-1,3,2-dioxaborolan-2-yl)pyrimidine (78 mg, 29.44%) as a brown oil.

LC-MS (ESI) m/z 312.1 [M+H]

### Step 5: 8-({4-[1-Cyclopropyl-4-(Trifluoromethyl)Imidazol-2-Yl]Phenyl}Methyl)-2-[4-Cyclopropyl-6-(Difluoromethoxy)Pyrimidin-5-Yl]Pyrido[2,3-d]Pyrimidin-7-One

To a stirred solution of 4-cyclopropyl-6-(difluoromethoxy)-5-(4,4,5,5-tetramethyl-1,3,2-dioxaborolan-2-yl)pyrimidine (30.0 mg, 0.096 mmol, 1.5 equiv) and 2-chloro-8-({4-[1-cyclopropyl-4-(trifluoromethyl)imidazo]-2-yl]phenyl}methyl)pyrido[2,3-d]pyrimidin-7-one (28.6 mg, 0.064 mmol, 1.00 equiv) in dioxane (1 mL) and H₂O (0.2 mL) were added K₃PO₄ (34.1 mg, mmol, 2.5 equiv) and Pd(dppf)Cl₂ (9.4 mg, 0.013 mmol, 0.2 equiv) at room temperature. The resulting mixture was stirred for 2 h at 90° C. under nitrogen atmosphere. The resulting mixture was filtered, the filter cake was washed with THF (2×3 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford the crude product. The crude product (26 mg) was purified by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 42% B to 60% B in 10 min; Wave Length: 220/254 nm; RT1(min): 10.98) to afford 84-{4-[1-cyclopropyl-4-(trifluoromethyl)imidazo]-2-yl]phenyl}methyl)-2-[4-cyclopropyl-6-(difluoromethoxy)pyrimidin-5-yl]pyrido[2,3-d]pyrimidin-7-one (12.3 mg, 32.23%) as an off-white solid.

LC-MS (ESI) m/z 595.95 [M+H]

¹H NMR: (400 MHz, DMSO-d6) δ 9.35 (d, J=0.9 Hz, 1H), 8.82 (s, 1H), 8.19 (dd, J=9.6, 0.9 Hz, 1H), 7.90 (s, 1H), 7.84-7.77 (m, 2H), 7.40 (d, J=8.1 Hz, 2H), 6.94 (d, J=9.6 Hz, 1H), 5.58 (s, 2H), 3.68 (td, J=6.8, 6.4, 3.5 Hz, 1H), 1.87 (tt, J=8.2, 4.6 Hz, 1H), 1.10-1.04 (m, 2H), 0.96 (dd, J=7.4, 5.2 Hz, 2H), 0.91 (t, J=4.5 Hz, 2H), 0.84 (dd, J=7.8, 3.5 Hz, 2H).

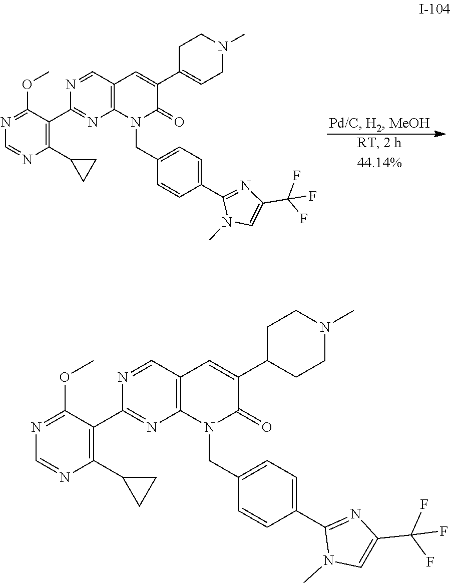

### 2-(4-Cyclopropyl-6-Methoxypyrimidin-5-Yl)-8-({4-[1-Methyl-4-(Trifluoromethyl)Imidazol-2-Yl]Phenyl}Methyl)-6-(1-Methylpiperidin-4-Yl)Pyrido[2,3-d]Pyrimidin-7-One

To a solution of 2-(4-cyclopropyl-6-methoxypyrimidin-5-yl)-6-(1-methyl-3,6-dihydro-2H-pyridin-4-yl)-8-({4-[1-methyl-4-(trifluoromethyl)imidazo]-2-yl]phenyl}methyl)pyrido[2,3-d]pyrimidin-7-one (12 mg, 0.019 mmol, 1 equiv) in MeOH (1 mL) was added Pd/C (5 mg, 10%) under nitrogen atmosphere. The mixture was hydrogenated at room temperature for 2 h under hydrogen atmosphere using a hydrogen balloon, filtered through a Celite pad and concentrated under reduced pressure. The residue was purified by reversed-phase flash chromatography (C18 silica gel column, eluting with 0% to 50% MeCN/water (0.1% FA) in 15 min with UV detector (254 nm)) to afford 2-(4-cyclopropyl-6-methoxypyrimidin-5-yl)-8-({4-[1-methyl-4-(trifluoromethyl)imidazo]-2-yl]phenyl}methyl)-6-(1-methylpiperidin-4-yl)pyrido[2,3-d]pyrimidin-7-one (5.4 mg, 44.14%) as an off-white solid.

LC-MS (ESI) m/z 631.35 [M+H].

¹H-NMR (400 MHz, DMSO-d6) δ 9.26 (s, 1H), 8.69 (s, 1H), 7.99 (s, 1H), 7.91 (d, J=1.4 Hz, 1H), 7.63 (d, J=8.0 Hz, 2H), 7.41 (d, J=7.9 Hz, 2H), 5.59 (s, 2H), 3.83 (s, 3H), 3.74 (s, 3H), 2.93-2.86 (m, 2H), 2.78 (td, J=11.8, 5.9 Hz, 1H), 2.20 (s, 3H), 1.98 (td, J=11.7, 2.3 Hz, 2H), 1.83 (d, J=12.6 Hz, 2H), 1.77-1.53 (m, 3H), 1.01 (p, J=3.5 Hz, 2H), 0.77 (dq, J=7.1, 3.5 Hz, 2H).

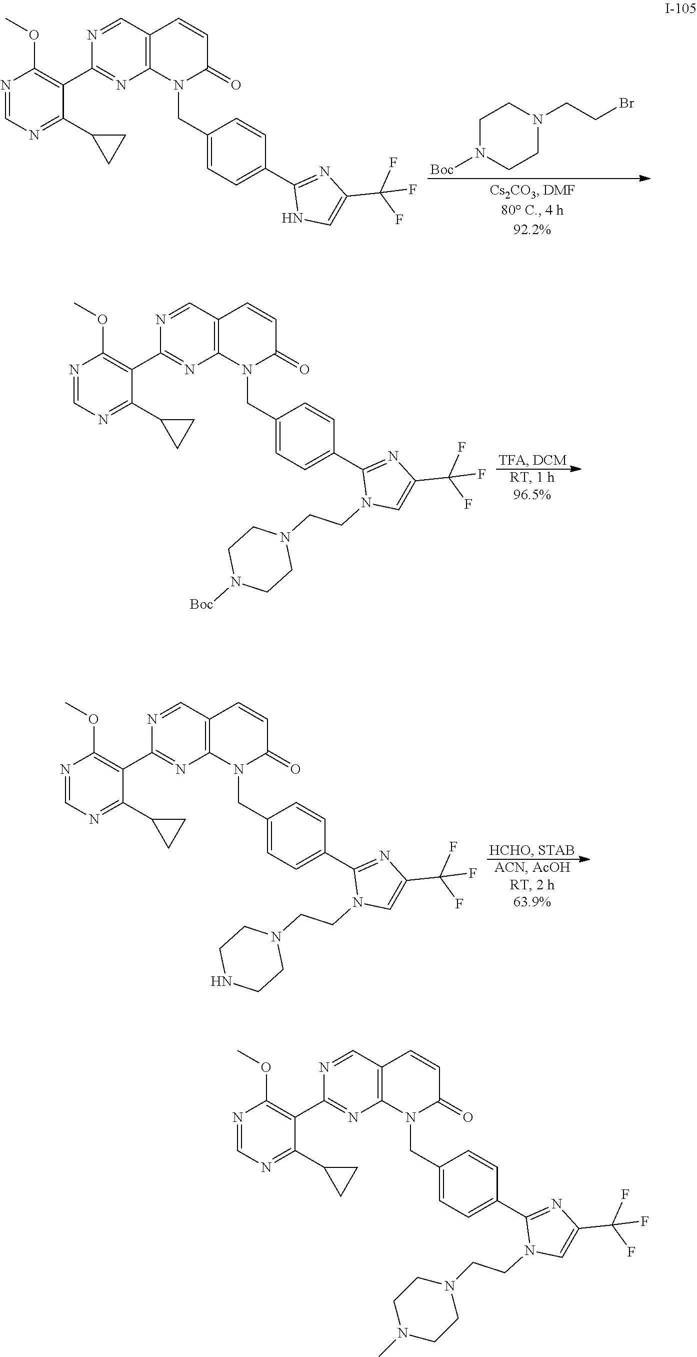

### Step 1: tert-butyl 4-{2-[2-(4-{12-(4-cyclopropyl-6-methoxypyrimidin-5-yl)-7-oxopyrido[2,3-d]pyrimidin-8-yl]methyl}phenyl)-4-(trifluoromethyl)imidazol-1-yl]ethyl}piperazine-1-carboxylate

A mixture of 2-(4-cyclopropyl-6-methoxypyrimidin-5-yl)-8-({4-[4-(trifluoromethyl)-1H-imidazol-2-yl]phenyl}methyl)pyrido[2,3-d]pyrimidin-7-one (100 mg, 0.192 mmol, 1 equiv), tert-butyl 4-(2-bromoethyl)piperazine-1-carboxylate (282.2 mg, 0.960 mmol, 5 equiv) and Cs₂CO₃ (188.16 mg, 0.576 mmol, 3 equiv) in DMF (2 mL, 25.843 mmol, 134.25 equiv) was stirred for 4 h at 80° C. under nitrogen atmosphere. The mixture was allowed to cool down to room temperature. The resulting mixture was diluted with water (2 mL). The resulting mixture was purified by reverse flash chromatography (C18 silica gel column, eluting with 0% to 100% MeCN/water (0.1% FA) in 10 min with UV detector (254 nm)) to afford tert-butyl 4-{2-[2-(4-{[2-(4-cyclopropyl-6-methoxypyrimidin-5-yl)-7-oxopyrido[2,3-d]pyrimidin-8-yl]methyl}phenyl)-4-(trifluoromethyl)imidazol-1-yl]ethyl}piperazine-1-carboxylate (130 mg, 92.29%) as a yellow solid.

LC-MS (ESI) m/z 732.3 [M+H]

### Step 2: 2-(4-Cyclopropyl-6-Methoxypyrimidin-5-Yl)-8-1(4-{1-[2-(Piperazin-1-Yl)Ethyl]-4-(Trifluoromethyl)Imidazol-2-Yl}Phenyl)Methyl]Pyrido[2,3-d]Pyrimidin-7-One

A mixture of tert-butyl 4-{24244-{[2-(4-cyclopropyl-6-m ethoxypyrimidin-5-yl)-7-oxopyrido[2,3-d]pyrimidin-8-yl]methyl}phenyl)-4-(trifluoromethyl)imidazol-1-yl]ethyl}piperazine-1-carboxylate (120 mg, 0.164 mmol, 1 equiv) in TFA (0.5 mL, 6.732 mmol, 41.05 equiv) and DCM (2 mL, 31.461 mmol, 191.86 equiv) was stirred for 1 h at room temperature under nitrogen atmosphere. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-8-[(4-{1-[2-(piperazin-1-yl)ethyl]-4-(trifluoromethyl)imidazol-2-yl}phenyl)methyl]pyrido[2,3-d]pyrimidin-7-one (100 mg, 96.54%) as a brown oil.

LC-MS (ESI) m/z 632.3 [M+H]

### Step 3: 2-(4-Cyclopropyl-6-Methoxypyrimidin-5-Yl)-8-1(4-{1-[2-(4-Methylpiperazin-1-Yl)Ethyl]-4-(Trifluoromethyl)Imidazol-2-Yl}Phenyl)Methyl]Pyrido[2,3-d]Pyrimidin-7-One

A solution of 2-(4-cyclopropyl-6-methoxypyrimidin-5-yl)-8-[(4-{1-[2-(piperazin-1-yl)ethyl]-4-(trifluoromethyl)imidazol-2-yl}phenyl)methyl]pyrido[2,3-d]pyrimidin-7-one (30 mg, 0.047 mmol, 1 equiv) and HCHO (11.5 mg, 0.141 mmol, 3 equiv, 37% in water) in ACN (0.6 mL) was treated with AcOH (28.5 mg, 0.470 mmol, 10 equiv) for 30 min at room temperature under nitrogen atmosphere followed by the addition of STAB (30.2 mg, 0.141 mmol, 3 equiv) at room temperature. The resulting mixture was stirred for 2 h at room temperature under nitrogen atmosphere. The resulting mixture was diluted with water (1 mL). The resulting mixture was purified by Prep-HPLC with the following conditions (Column: XBridge Shield RP18 OBD Column, 30*150 mm, 51 μm; Mobile Phase A: Water(10 mmol/L NH 4 HCO 3), Mobile Phase B: ACN; Flow rate: 50 mL/min; Gradient: 32% B to 44% B in 8 min, 44% B; Wave Length: 254/220 nm; RT1(min): 7.09) to afford 2-(4-cyclopropyl-6-methoxypyrimidin-5-yl)-8-[(4-{1-[2-(4-methylpiperazin-1-yl)ethyl]-4-(trifluoromethyl)imidazol-2-yl}phenyl)methyl]pyrido[2,3-d]pyrimidin-7-one (19.9 mg, 63.92%) as an off-white solid.

LC-MS (ESI) m/z 646.3 [M+H]

¹H NMR (400 MHz, DMSO-d6) δ 9.29 (s, 1H), 8.69 (s, 1H), 8.16 (d, J=9.5 Hz, 1H), 7.97 (d, J=1.4 Hz, 1H), 7.59-7.52 (m, 2H), 7.45-7.38 (m, 2H), 6.90 (d, J=9.5 Hz, 1H), (s, 2H), 4.10 (t, J=6.2 Hz, 2H), 3.83 (s, 3H), 2.54 (m, J=6.2 Hz, 2H), 2.35-1.99 (m, 10H), 1.74 (m J=8.3, 4.7 Hz, 1H), 1.02 (m, J=6.2, 3.4 Hz, 2H), 0.79 (m, J=8.1, 3.4 Hz, 2H).

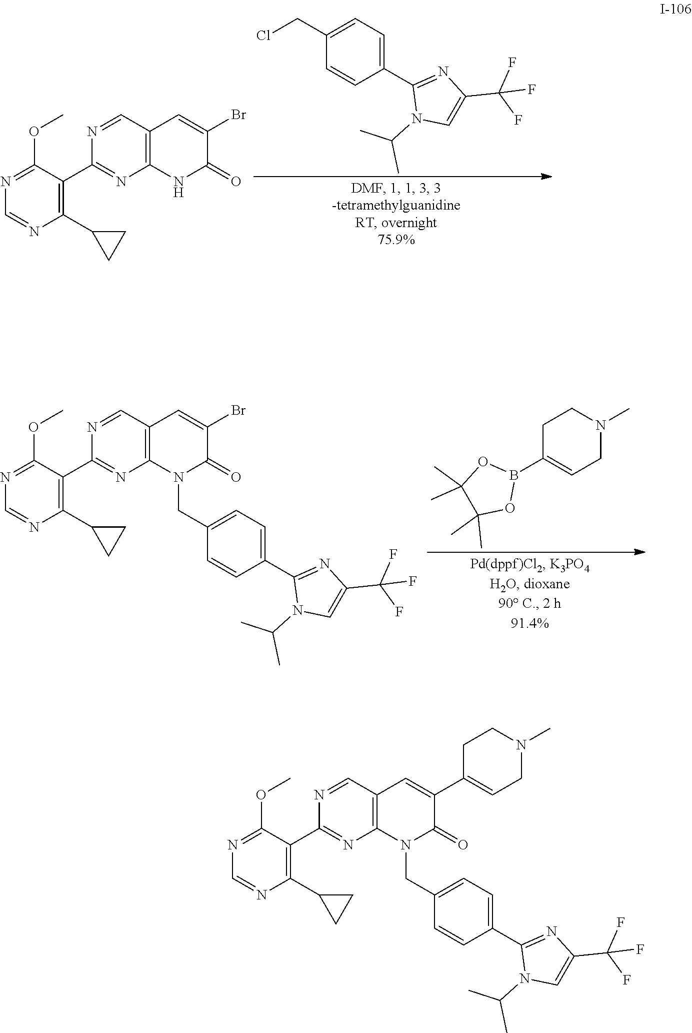

### Step 1: 6-Bromo-2-(4-Cyclopropyl-6-Methoxypyrimidin-5-Yl)-8-({4-[1-Isopropyl-4-(Trifluoromethyl)Imidazol-2-Yl]Phenyl}Methyl)Pyrido[2,3-d]Pyrimidin-7-One

To a stirred mixture of 6-bromo-2-(4-cyclopropyl-6-methoxypyrimidin-5-yl)-8H-pyrido[2,3-d]pyrimidin-7-one (100 mg, 0.267 mmol, 1 equiv) and 2-[4-(chloromethyl)phenyl]-1-isopropyl-4-(trifluoromethyl)imidazole (80.9 mg, 0.267 mmol, 1 equiv) in dimethylformamide (2 mL) was added 1,1,3,3-tetramethylguanidine (46.2 mg, 0.401 mmol, 1.5 equiv) dropwise at room temperature under nitrogen atmosphere. The resulting mixture was stirred for overnight at room temperature under nitrogen atmosphere. The resulting mixture was purified by reverse flash chromatography (C18 silica gel column, eluting with 0% to 100% MeCN/water (0.1% FA) in 10 min with UV detector (254 nm)) to afford 6-bromo-2-(4-cyclopropyl-6-methoxypyrimidin-5-yl)-8-({4-[1-isopropyl-4-(trifluoromethyl)imidazol-2-yl]phenyl}methyl)pyrido[2,3-d]pyrimidin-7-one (130 mg, 75.95%) as an off-white solid.

LC-MS (ESI) m/z 640.1 [M+H]

### Step 2: 2-(4-Cyclopropyl-6-Methoxypyrimidin-5-Yl)-8-({4-[1-Isopropyl-4-(Trifluoromethyl)Imidazol-2-Yl]Phenyl}Methyl)-6-(1-Methyl-3,6-Dihydro-211-Pyridin-4-Yl)Pyrido[2,3-d]Pyrimidin-7-One

To a stirred mixture of 6-bromo-2-(4-cyclopropyl-6-methoxypyrimidin-5-yl)-8-({4-[1-isopropyl-4-(trifluoromethyl)imidazol-2-yl]phenyl}methyl)pyri do[2,3-d]pyrimidin-7-one (30 mg, 0.047 mmol, 1 equiv) and 1-methyl-4-(4,4,5,5-tetramethyl-1,3,2-dioxaborolan-2-yl)-3,6-dihydro-2H-pyridine (15.7 mg, 0.071 mmol, 1.5 equiv) in H₂O (0.15 mL, 8.326 mmol, 177.76 equiv) and dioxane (0.6 mL, 7.082 mmol, 151.20 equiv) were added K₃PO₄ (24.9 mg, 0.117 mmol, 2.5 equiv) and Pd(dppf)C12 (6.8 mg, 0.009 mmol, 0.2 equiv) at room temperature under nitrogen atmosphere. The resulting mixture was stirred for 2h at under nitrogen atmosphere. The mixture was allowed to cool down to room temperature. The resulting mixture was filtered, the filter cake was washed with acetonitrile (3×2 mL). The filtrate was concentrated under reduced pressure. The residue was purified by reverse flash chromatography (C18 silica gel column, eluting with 0% to 100% MeCN/water (10 mmol/L NH₄HCO₃) in 10 min with UV detector (254 nm)) to afford 2-(4-cyclopropyl-6-methoxypyrimidin-5-yl)-8-({4-[1-isopropyl-4-(trifluoromethyl)imidazol-2-yl]phenyl}methyl)-6-(1-methyl-3,6-dihydro-2H-pyridin-4-yl)pyrido[2,3-d]pyrimidin-7-one (28.6 mg, 91.49%) as a yellow solid.

LC-MS (ESI) m/z 657.3 [M+H]

¹H NMR (400 MHz, DMSO-d6) δ 9.28 (s, 1H), 8.69 (s, 1H), 8.16 (s, J=1.4 Hz, 1H), 8.09 (s, 1H), 7.48 (d, J=8.2 Hz, 2H), 7.41 (d, J=8.2 Hz, 2H), 6.78 (s, 1H), 5.61 (s, 2H), 4.41 (m, J=6.4 Hz, 1H), 3.83 (s, 3H), 3.07 (s, 2H), 2.71-2.53 (m, 4H), 2.29 (s, 3H), 1.73 (m, J=8.0, 4.3 Hz, 1H), 1.37 (d, J=6.6 Hz, 6H), 1.00 (m, J=4.4 Hz, 2H), 0.75 (m, J=8.0, 3.3 Hz, 2H).

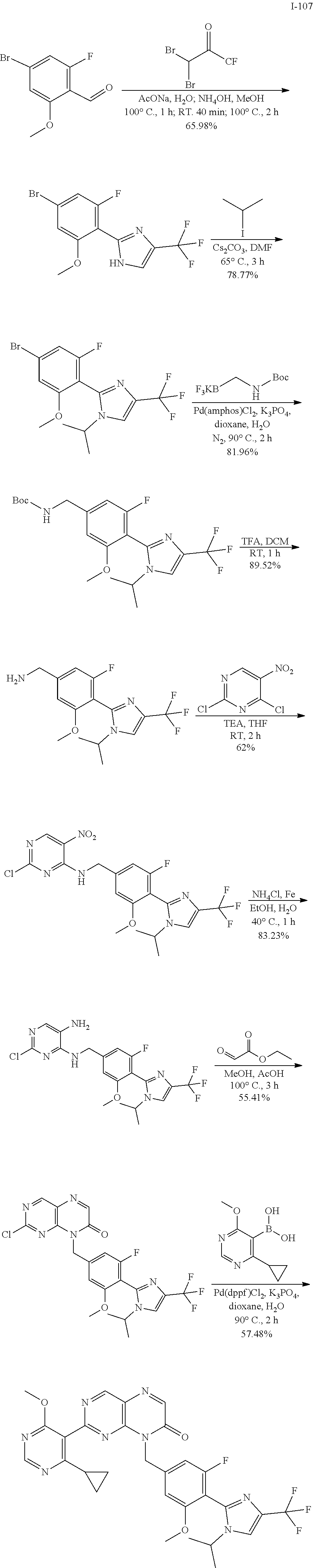

### Step 1: 2-(4-Bromo-2-Fluoro-6-Methoxyphenyl)-4-(Trifluoromethyl)-1H-Imidazole

To a stirred solution of 3,3-dibromo-1,1,1-trifluoropropan-2-one (12.97 g, 48.061 mmol, 1.12 equiv) in H₂O (20.00 mL, 1110.133 mmol, 25.87 equiv) was added AcONa(3.94 g, 48.061 mmol, 1.12 equiv) at room temperature. The resulting mixture was stirred for 1h at 100° C. under nitrogen atmosphere. The mixture was allowed to cool down to room temperature. To the above mixture was added a solution of 4-bromo-2-fluoro-6-methoxybenzaldehyde (10 g, 42.912 mmol, 1 equiv) in NH 4 OH (33.34 mL, 856.094 mmol, 19.95 equiv) and MeOH(150.01 mL, 3705.022 mmol, 86.34 equiv) at room temperature. The resulting mixture was stirred for additional 40 min at room temperature. Then, the reaction was warmed to 100° C. and stirred for 2h. After completion of reaction, the mixture was allowed to cool down to room temperature. The reaction mixture was concentrated under reduced pressure. Then the mixture was quenched by addition of water 40 mL. The aqueous layer was extracted with ethyl acetate (3×30 mL). The combined organic phase was dried over anhydrous sodium sulfate and concentrated under reduced pressure. The residue was purified by silica gel column chromatography (eluting with PE/EA (3:1)) to afford 2-(4-bromo-2-fluoro-6-methoxyphenyl)-4-(trifluoromethyl)-1H-imidazole (9.6 g, as a yellow solid.

LC-MS (ESI) m/z 340.0 [M+H]

### Step 2: 2-(4-Bromo-2-Fluoro-6-Methoxyphenyl)-1-Isopropyl-4-(Trifluoromethyl)Imidazole

To a stirred solution of 2-(4-bromo-2-fluoro-6-methoxyphenyl)-4-(trifluoromethyl)-1H-imidazole(9.6 g, 28.311 mmol, 1 equiv) in DMF(200 mL) was added Cs2CO3 (18.51 g, 56.622 mmol, 2 equiv) at room temperature. The resulting mixture was stirred for 5 min at room temperature under nitrogen atmosphere. To the above mixture was added 2-iodopropane (14.44 g, 84.933 mmol, 3 equiv) at room temperature. The resulting mixture was stirred for additional 3 h at 65° C. The mixture was allowed to cool down to room temperature. The reaction was diluted with Water (20 mL) at room temperature. The aqueous layer was extracted with EtOAc (3×20 mL). The combined organic layers were washed with brine (2×30 mL), dried over anhydrous Na₂SO₄ and concentrated under reduced pressure. The residue was purified by silica gel column chromatography (eluting with PE/EA (9:1)) to afford 2-(4-bromo-2-fluoro-6-methoxyphenyl)-1-isopropyl-4-(trifluoromethyl)imidazole (8.5 g, 78.77%) as a yellow oil.

LC-MS (ESI) m/z 382.1 [M+H]

### Step 3: Tert-Butyl N-({3-Fluoro-4-[1-Isopropyl-4-(Trifluoromethyl)Imidazol-2-Yl]-5-Methoxyphenyl}Methyl)Carbamate

To a stirred solution of 2-(4-bromo-2-fluoro-6-methoxyphenyl)-1-isopropyl-4-(trifluoromethyl)imidazole (8.3 g, 21.775 mmol, 1 equiv) and tert-butyl N-{[trifluoro(potassio)-lambda5-boranyl]methyl}carbamate (6.19 g, 26.130 mmol, 1.2 equiv) in dioxane (166.01 mL, 1959.532 mmol, 89.99 equiv) and H₂O (33.20 mL, 1842.818 mmol, 84.63 equiv) were added K₃PO₄ (9.24 g, 43.550 mmol, 2 equiv) and Pd(amphos)Cl₂ (1.54 g, 2.177 mmol, 0.1 equiv) at room temperature. The resulting mixture was stirred for 2 h at 90° C. under nitrogen atmosphere. The mixture was allowed to cool down to room temperature. The reaction was diluted with Water (20 mL) at room temperature. The resulting mixture was extracted with EtOAc (3×30 mL). The combined organic layers were washed with brine (3×20 mL), dried over anhydrous Na₂SO₄. After filtration, the filtrate was concentrated under reduced pressure. The residue was purified by silica gel column chromatography (eluting with PE/EA (5:1)) to afford tert-butyl N-({3-fluoro-4-[1-isopropyl-4-(trifluoromethyl)imidazo]-2-yl]-5-methoxyphenyl methyl)carbamate (7.7 g, 81.96%) as a yellow oil.

LC-MS (ESI) m/z 432.4 [M+H]

### Step 4: 1-{3-Fluoro-4-[1-Isopropyl-4-(Trifluoromethyl)Imidazol-2-Yl]-5-Methoxyphenyl}Methanamine

A solution of tert-butyl N-({3-fluoro-4-[1-isopropyl-4-(trifluoromethyl)imidazo]-2-yl]-5-methoxyphenyl}methyl)carbamate (3.2 g, 7.417 mmol, 1 equiv) in DCM (59.26 mL, 932.243 mmol, 125.69 equiv) was added TFA (9.60 mL, 129.278 mmol, 17.43 equiv) at room temperature under nitrogen atmosphere. The resulting mixture was stirred for additional 1 h at room temperature. The resulting mixture was concentrated under reduced pressure. The reaction was diluted with sat.NaHCO₃ (20 mL) at room temperature. The resulting mixture was extracted with DCM (3×20 mL). The combined organic layers were washed with brine (3×mL), dried over anhydrous Na₂SO₄. After filtration, the filtrate was concentrated under reduced pressure. The residue was purified by silica gel column chromatography(eluting with PE/EA (1:1)) to afford 1-{3-fluoro-4-[1-isopropyl-4-(trifluoromethyl)imidazol-2-yl]-5-methoxyphenyl methanamine(2.2 g, 89.52%) as a yellow oil.

LC-MS (ESI) m/z 332.3 [M+H]

### Step 5. 2-Chloro-N-({3-Fluoro-4-[1-Isopropyl-4-(Trifluoromethyl)Imidazol-2-Yl]-5-Methoxyphenyl}Methyl)-5-Nitropyrimidin-4-Amine

To a stirred solution of 1-{3-fluoro-4-[1-isopropyl-4-(trifluoromethyl)imidazo]-2-yl]-5-methoxyphenyl}methanamine (4.7 g, 14.186 mmol, 1 equiv) and 2,4-dichloro-5-nitropyrimidine (5.50 g, 28.372 mmol, 2 equiv) in THF (94.00 mL, 1160.273 mmol, 81.79 equiv) was added TEA (4.31 g, 42.558 mmol, 3 equiv) at room temperature. The resulting mixture was stirred for 2 h at room temperature. The resulting mixture was concentrated under reduced pressure. The reaction was diluted with Water (20 mL) at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography(eluting with PE/EA (1:1)) to afford 2-chloro-N-({3-fluoro-4-[1-isopropyl-4-(trifluoromethyl)imidazo]-2-yl]-5-methoxyphenyl}methyl)-5-nitropyrimidin-4-amine (4 g, 57.68%) as a yellow oil.

LC-MS (ESI) m/z 312.1 [M+H]

### Step 6: 2-Chloro-N4-({3-Fluoro-4-[1-Isopropyl-4-(Trifluoromethyl)Imidazol-2-Yl]-5-Methoxyphenyl}Methyl)Pyrimidine-4,5-Diamine

To a stirred solution of 2-chloro-N-({3-fluoro-4-[1-isopropyl-4-(trifluoromethyl)imidazo]-2-yl]-5-methoxyphenyl methyl)-5-nitropyrimidin-4-amine (3.2 g, 6.546 mmol, 1 equiv) in EtOH (64.00 mL, 1101.626 mmol, 168.29 equiv) and H₂O (16.00 mL, 888.096 mmol, 135.67 equiv) were added NH 4 C1 (1.75 g, 32.730 mmol, 5 equiv) and Fe (2.92 g, 52.368 mmol, 8 equiv) at room temperature. The resulting mixture was stirred for 1 h at 40° C. The resulting mixture was filtered through a Celite pad, the filter cake was washed with EtOAc (2×30 mL). The filtrate was concentrated under reduced pressure to afford 2-chloro-N4-({3-fluoro-4-[1-isopropyl-4-(trifluoromethyl)imidazo]-2-yl]-5-methoxyphenyl methyl)pyrimidine-4,5-di amine (2.5 g, 83.23%) as a dark green solid.

LC-MS (ESI) m/z 459.8 [M+H]

### Step 7. 2-Chloro-8-({3-Fluoro-4-[1-Isopropyl-4-(Trifluoromethyl)Imidazol-2-Yl]-5-Methoxyphenyl}Methyl)Pyrimidine-7-One

To a stirred solution of 2-chloro-N4-({3-fluoro-4-[isopropyl-4-(trifluoromethyl)imidazol-2-yl]-5-methoxyphenyl}methyl)pyrimidine-4,5-diamine (1.5 g, 3.269 mmol, 1 equiv) in MeOH (30 mL) was added ethyl glyoxylate (1.00 g, 9.807 mmol, 3 equiv) at room temperature. To the above mixture was added AcOH (0.5 uL) at room temperature. The resulting mixture was stirred for 3 h at 100° C. The mixture was allowed to cool down to room temperature. The resulting mixture was concentrated under reduced pressure. The residue was dissolved in water (20 mL). The resulting mixture was extracted with EtOAc (3×30 mL). The combined organic layers were washed with brine (3×10 mL), dried over anhydrous Na₂SO₄. After filtration, the filtrate was concentrated under reduced pressure. The residue was purified by silica gel column chromatography(eluting with PE/EA (3:1)) to afford 2-chloro-8-({3-fluoro-4-[1-isopropyl-4-(trifluoromethyl)imidazol-2-yl]-5-methoxyphenyl methyl)pteridin-7-one (900 mg, 55.41%) as a yellow oil.

LC-MS (ESI) m/z 497.8 [M+H]

### Step 8. 2-(4-Cyclopropyl-6-Methoxypyrimidin-5-Yl)-8-({3-Fluoro-4-[1-Isopropyl-4-(Trifluoromethyl)Imidazol-2-Yl]-5-Methoxyphenyl}Methyl)Pteridin-7-One

To a stirred solution of 2-chloro-8-({3-fluoro-4-[1-isopropyl-4-(trifluoromethyl)imidazol-2-yl]-5-methoxyphenyl}methyl)pteridin-7-one (150 mg, 0.302 mmol, 1 equiv) and 4-cyclopropyl-6-methoxypyrimidin-5-ylboronic acid (87.85 mg, 0.453 mmol, 1.5 equiv) in dioxane (3 mL) and H₂O (0.6 mL) were added Pd(dppf)C12 (44.18 mg, 0.060 mmol, 0.2 equiv) and K₃PO₄ (160.21 mg, 0.755 mmol, 2.5 equiv) at room temperature. The resulting mixture was stirred for 2 h at 90° C. under nitrogen atmosphere. The mixture was allowed to cool down to room temperature. The resulting mixture was filtered, the filter cake was washed with EtOAc (2×5 mL). The filtrate was concentrated under reduced pressure. The residue was diluted with DMSO (2 mL). The resulting mixture was purified by reversed-phase flash chromatography (C18 silica gel column, eluting with 0% to 100% MeCN/water (0.1% FA) in 10 min with UV detector (254 nm)) to afford the crude product. The crude product was purified by Prep-HPLC with the following conditions (Column: XBridge Prep OBD C18 Column, 30*150 mm, 51 μm; Mobile Phase A: Water(10 mmol/L NH₄HCO₃), Mobile Phase B: ACN; Flow rate: 60 mL/min; Gradient: 40% B to 58% B in 10 min, 58% B; Wave Length: 220/254 nm; RT1(min): 10.55) to afford 2-(4-cyclopropyl-6-methoxypyrimidin-5-yl)-8({3-fluoro-4-[1-isopropyl-4-(trifluoromethyl)imidazol-2-yl]-5-methoxyphenyl}methyl)pteridin-7-one (50.5 mg, 27.15%) as a white solid

LC-MS (ESI) m/z 611.02 [M+H]

¹H NMR: (400 MHz, DMSO-d6) δ 9.38 (s, 1H), 8.70 (s, 1H), 8.49 (s, 1H), 8.18 (q, J=1.2 Hz, 1H), 7.08 (s, 1H), 6.93-6.86 (m, 1H), 5.47 (s, 2H), 3.91 (h, J=6.6 Hz, 1H), 3.83 (s, 3H), 3.72 (s, 3H), 1.77 (tt, J=8.2, 4.6 Hz, 1H), 1.36 (d, J=6.6 Hz, 3H), 1.24 (d, J=6.7 Hz, 3H), 1.03 (d, J=3.9 Hz, 2H), 0.84-0.74 (m, 2H).

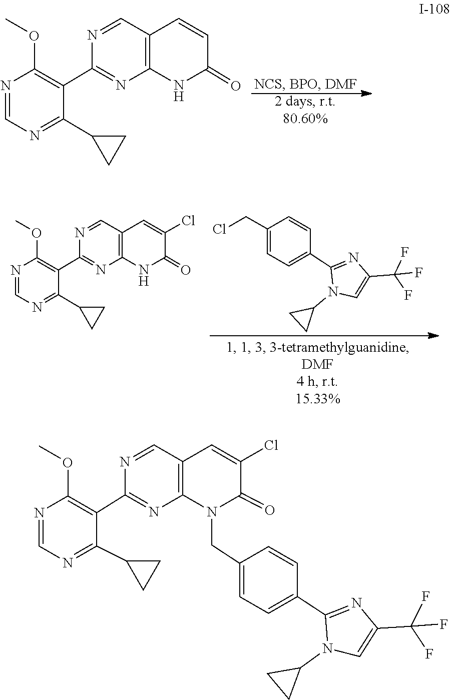

### Step 1: 6-Chloro-2-(4-Cyclopropyl-6-Methoxypyrimidin-5-Yl)-8H-Pyrido[2,3-d]Pyrimidin-7-One

To a stirred solution of 2-(4-cyclopropyl-6-methoxypyrimidin-5-yl)-8H-pyrido[2,3-d]pyrimidin-7-one (100 mg, 0.339 mmol, 1 equiv) and DMF (1 mL, 12.922 mmol, 38.16 equiv) were added NCS (90.4 mg, 0.678 mmol, 2 equiv) and BPO (8.6 mg, 0.034 mmol, 0.1 equiv) at room temperature under nitrogen atmosphere. The resulting mixture was stirred for additional 2 days at room temperature. The resulting mixture was diluted with H₂O (10 mL). The resulting mixture was extracted with EtOAc (3×10 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% FA) with UV detector (254 nm)) to afford 6-chloro-2-(4-cyclopropyl-6-methoxypyrimidin-5-yl)-8H-pyrido[2,3-d]pyrimidin-7-one (90 mg, 80.60%) as a yellow oil.

LC-MS (ESI) m/z 330 [M+H]

### Step 2: 6-Chloro-8-({4-[1-Cyclopropyl-4-(Trifluoromethyl)Imidazol-2-Yl]Phenyl}Methyl)-2-(4-Cyclopropyl-6-Methoxypyrimidin-5-Yl)Pyrido[2,3-d]Pyrimidin-7-One

To a stirred solution of 6-chloro-2-(4-cyclopropyl-6-methoxypyrimidin-5-yl)-8H-pyrido[2,3-d]pyrimidin-7-one (45 mg, 0.136 mmol, 1.2 equiv) and 2-[4-(chloromethyl)phenyl]-1-cyclopropyl-4-(trifluoromethyl)imidazole (34.20 mg, 0.113 mmol, 1 equiv) were added 1,1,3,3-tetramethylguanidine (39.30 mg, 0.340 mmol, 3 equiv) and DMF (1 mL, 12.922 mmol, 94.68 equiv) at room temperature under nitrogen atmosphere. The resulting mixture was stirred for additional 4 h at room temperature. The resulting mixture was diluted with H₂O (10 mL). The resulting mixture was extracted with EtOAc (3×20 mL). The combined organic layers were washed with brine (3×10 mL), dried over anhydrous Na₂SO₄, filtered and concentrated under reduced pressure. The residue was purified by reverse flash chromatography (C18 silica gel column, eluting with 0% to 100% in 10 min MeCN/water (0.1% FA) with UV detector (254 nm)) to afford 6-chloro-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (10.6 mg, 15.33%) as an off-white solid.

LC-MS (ESI) m/z 594 [M+H]

¹H NMR (400 MHz, DMSO-d6) δ 9.29 (s, 1H), 8.69 (s, 1H), 8.58 (s, 1H), 7.92 (d, J=1.4 Hz, 1H), 7.85-7.78 (m, 2H), 7.41 (d, J=8.3 Hz, 2H), 5.63 (s, 2H), 3.83 (s, 3H), 3.70 (tt, J=7.1, 3.9 Hz, 1H), 1.74 (tt, J=8.1, 4.6 Hz, 1H), 1.02 (q, J=3.9 Hz, 2H), 0.96 (dd, J=7.4, 4.9 Hz, 2H), 0.96-0.85 (m, 2H), 0.77 (dq, J=7.0, 3.5 Hz, 2H).

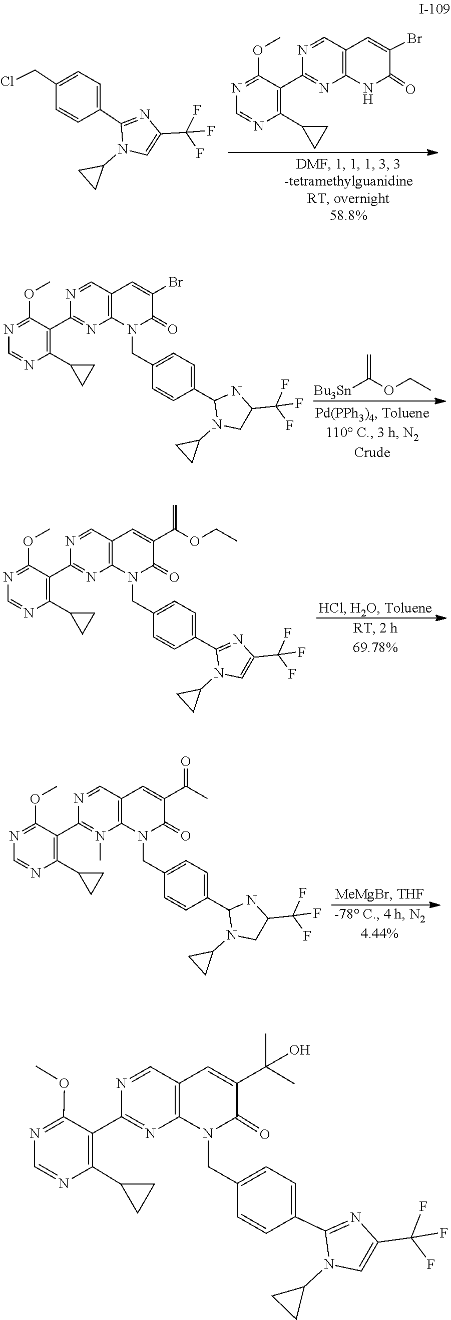

### 6-Bromo-8-({4-[1-Cyclopropyl-4-(Trifluoromethyl)Imidazol-2-Yl]Phenyl}Methyl)-2-(4-Cyclopropyl-6-Methoxypyrimidin-5-Yl)Pyrido[2,3-d]Pyrimidin-7-One

To a stirred mixture of 2-[4-(chloromethyl)phenyl]-1-cyclopropyl-4-(trifluoromethyl)imidazole (100 mg, 0.333 mmol, 1 equiv) and 6-bromo-2-(4-cyclopropyl-6-methoxypyrimidin-5-yl)-8H-pyrido[2,3-d]pyrimidin-7-one (124.4 mg, 0.333 mmol, 1 equiv) in DMF (2 mL) was added 1,1,3,3-tetramethylguanidine (57.4 mg, 0.500 mmol, 1.5 equiv) at room temperature under nitrogen atmosphere. The resulting mixture was stirred for overnight at room temperature under nitrogen atmosphere. The resulting mixture was purified by reverse flash chromatography (C18 silica gel column, eluting with 0% to 100% MeCN/water (0.1% FA) in 10 min with UV detector (254 nm)) to afford 6-bromo-8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (125 mg, 58.88%) as an off-white solid.

Step 2:

To a stirred solution of 6-bromo-8-({4-[1-cyclopropyl-4-(trifluoromethyl)imidazol-2-yl]phenyl methyl)-2-(4-cyclopropyl-6-methoxypyrimidin-5-yl)pyrido[2,3-d]pyrimidin-7-one (30 mg, 0.047 mmol, 1 equiv) and tributyl(1-ethoxyethenyl)stannane (20.4 mg, 0.056 mmol, 1.2 equiv) in Toluene (1 mL) was added Pd(PPh₃)₄ (5.5 mg, 0.005 mmol, 0.1 equiv) at room temperature. The resulting mixture was stirred for 3 h at 110° C. under nitrogen atmosphere. After completion of reaction, the resulting mixture was concentrated under reduced pressure. The crude product was used in the next step directly without further purification.

LC-MS (ESI) m/z 630.6 [M+H]

### Step 3: 6-Acetyl-8-({4-[1-Cyclopropyl-4-(Trifluoromethyl)Imidazol-2-Yl]Phenyl}Methyl)-2-(4-Cyclopropyl-6-Methoxypyrimidin-5-Yl)Pyrido[2,3-d]Pyrimidin-7-One

A solution of 8-({4-[1-cyclopropyl-4-(trifluoromethyl)imidazo]-2-yl]phenyl}methyl)-2-(4-cyclopropyl-6-methoxypyrimidin-5-yl)-6-(1-ethoxyethenyl)pyrido[2,3-d]pyrimidin-7-one (30 mg, 0.048 mmol, 1 equiv) in 2M HCl (1 mL) and Toluene (1 mL) was stirred for 2 h at room temperature. The mixture was neutralized to pH 7 with saturated NaHCO₃(aq.). The resulting mixture was extracted with EtOAc (3×15 mL). The combined organic layers were washed with brine (2×20 mL), dried over anhydrous Na₂SO₄, filtered and concentrated under reduced pressure. The residue was purified by reversed-phase flash chromatography (C18 silica gel column, eluting with 0% to 100% MeCN/water (0.1% FA) in 20 min with UV detector (254 nm)) to afford 6-acetyl-8-({4-[1-cyclopropyl-4-(trifluoromethyl)imidazo]-2-yl]phenyl methyl)-2-(4-cyclopropyl-6-methoxypyrimidin-5-yl)pyri do[2,3-d]pyrimidin-7-one (20 mg, 69.78%) as a light yellow solid.

LC-MS (ESI) m/z 602.5 [M+H]

### Step 4: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(2-Hydroxypropan-2-Yl)Pyrido[2,3-d]Pyrimidin-7-One)

To a stirred solution of 6-acetyl-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (37 mg, 0.062 mmol, 1 equiv) in THF (1 mL) was added MeMgBr (0.06 mL, 0.186 mmol, 3 equiv, 3 M in THF) dropwise at −78° C. under nitrogen atmosphere. The resulting mixture was stirred for 3 h at −78° C. under nitrogen atmosphere. The resulting mixture was stirred for 4 h at −78° C. under nitrogen atmosphere. The reaction was quenched with sat. NH₄Cl (aq.) at room temperature. The aqueous layer was extracted with EtOAc (3×15 mL). The combined organic layers were dried over anhydrous Na₂SO₄, filtered and concentrated under reduced pressure. The residue was purified by Prep-TLC (CH₂Cl₂/MeOH (20:1)) to afford the crude product. The crude product was purified by Prep-HPLC with the following conditions (Column: XBridge Shield RP18 OBD Column, 19*250 mm, 10 μm; Mobile Phase A: Water(0.05% NH₃.H₂O), Mobile Phase B: ACN; Flow rate: 50 mL/min; Gradient: 30% B to 50% B in 10 min, 50% B; Wave Length: 254/220 nm; RT1(min): 10.42) to afford 8-({4-[1-cyclopropyl-4-(trifluoromethyl)imidazo]-2-yl]phenyl methyl)-2-(4-cyclopropyl-6-methoxypyrimidin-5-yl)-6-(2-hydroxypropan-2-yl)pyrido[2,3-d]pyrimidin-7-one) (1.7 mg, 4.44%) as an off-white solid.

LC-MS (ESI) m/z 618.30 [M+H]

¹H NMR: (400 MHz, DMSO-d6) δ 9.35 (s, 1H), 8.69 (s, 1H), 8.30 (s, 1H), 7.93-7.88 (m, 1H), 7.81 (d, J=8.3 Hz, 2H), 7.39 (d, J=8.1 Hz, 2H), 5.60 (s, 2H), 5.44 (s, 1H), 3.83 (d, J=0.9 Hz, 3H), 3.70 (tt, J=7.3, 4.0 Hz, 1H), 1.74 (tt, J=8.2, 4.6 Hz, 1H), 1.54 (s, 6H), 1.01 (t, J=3.8 Hz, 2H), 0.95 (dd, J=7.6, 4.6 Hz, 2H), 0.92-0.85 (m, 2H), 0.77 (dd, J=7.9, 3.5 Hz, 2H).

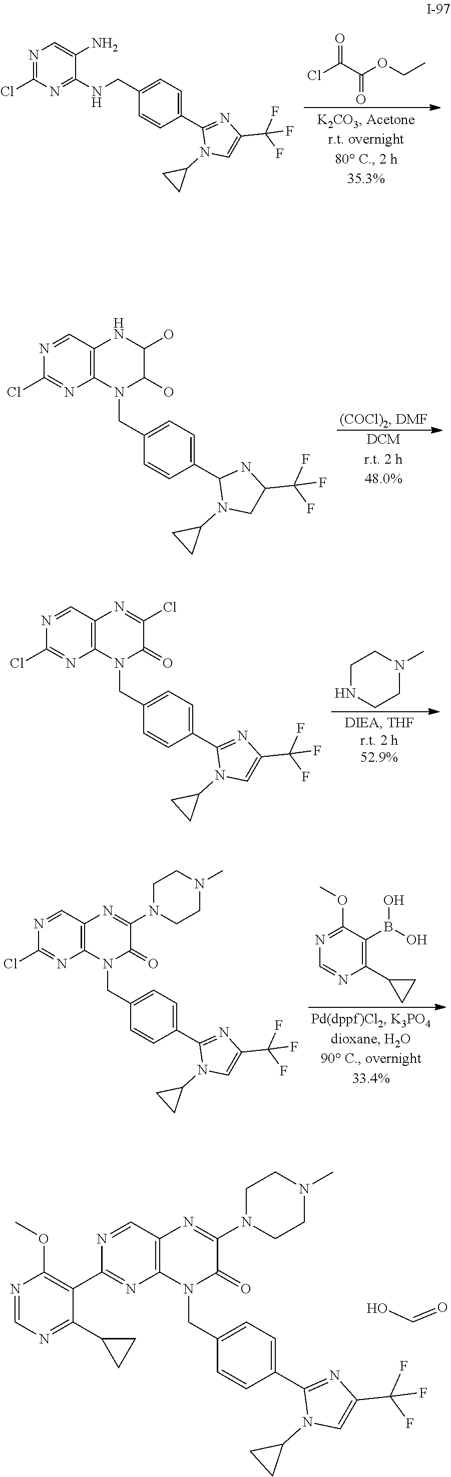

### Step 1: 2-Chloro-8-({4-[1-Cyclopropyl-4-(Trifluoromethyl)Imidazol-2-Yl]Phenyl}Methyl)-511-Pteridine-6,7-Dione

To a stirred solution of 2-chloro-N4-({4-[1-cyclopropyl-4-(trifluoromethyl)imidazol-2-yl]phenyl}methyl)pyrimidine-4,5-diamine (200 mg, 0.489 mmol, 1 equiv) and K₂CO₃ (169.0 mg, 1.222 mmol, 2.5 equiv) in Acetone (2 mL, 27.204 mmol, 55.61 equiv) was added ethyl chloroglyoxylate (133.5 mg, 0.978 mmol, 2 equiv) at room temperature. The resulting mixture was stirred overnight at room temperature and then for 2 h at 80° C. The mixture was allowed to cool down to room temperature. The resulting mixture was filtered, the filter cake was washed with acetone (3×5 mL). The filtrate was concentrated under reduced pressure. The residue was purified by reverse flash chromatography (C18 silica gel column, eluting with 0% to 100% MeCN/water (0.1% TFA) in 10 min with UV detector (254 nm)) to afford 2-chloro-84 {4-[1-cyclopropyl-4-(trifluoromethyl)imidazo]-2-yl]phenyl methyl)-5H-pteridine-6, 7-dione (80 mg, 35.33%) as an off-white solid.

LC-MS (ESI) m/z 463.8 [M+H].

### Step 2: 2,6-Dichloro-8-({4-[1-Cyclopropyl-4-(Trifluoromethyl)Imidazol-2-Yl]Phenyl}Methyl)Pteridin-7-One

To a stirred solution of 2-chloro-8-({4-[1-cyclopropyl-4-(trifluoromethyl)imidazol-2-yl]phenyl}methyl)-5H-pteridine-6,7-dione (100 mg, 0.216 mmol, 1 equiv) in DCM (1 mL) was added (COCl)₂ (54.8 mg, 0.432 mmol, 2 equiv) followed by the addition of DMF (0.1 mL) at room temperature. The resulting mixture was stirred for 2 h at room temperature. The reaction was quenched by the addition of Water (10 mL) at room temperature. The resulting mixture was extracted with CH₂Cl₂ (3×10 mL). The combined organic layers were dried over anhydrous Na₂SO₄, filtered and concentrated under reduced pressure. The residue was purified by reverse flash chromatography (C18 silica gel column, eluting with 0% to 100% MeCN/water (0.1% TFA) in 10 min with UV detector (254 nm)) to afford 2,6-dichloro-8-({4-[1-cyclopropyl-4-(trifluoromethyl)imidazo]-2-yl]phenyl}methyl)pteridin-7-one (50 mg, 48.0%) as a colorless oil.

LC-MS (ESI) m/z 481.0 [M+H].

### Step 3: 2-Chloro-8-({4-[1-Cyclopropyl-4-(Trifluoromethyl)Imidazol-2-Yl]Phenyl}Methyl)-6-(4-Methylpiperazin-1-Yl)Pteridin-7-One

To a stirred solution of 2,6-dichloro-8-({4-[1-cyclopropyl-4-(trifluoromethyl)imidazol-2-yl]phenyl}methyl)pteridin-7-one (50 mg, 0.104 mmol, 1 equiv) in THF (2 mL) was added DIEA (26.8 mg, 0.208 mmol, 2 equiv) followed by the addition of 1-methyl-piperazine (12.4 mg, 0.125 mmol, 1.2 equiv) at room temperature. The resulting mixture was stirred for 2 h at room temperature. The resulting mixture was concentrated under reduced pressure. The residue was purified by reverse flash chromatography (C18 silica gel column, eluting with 0% to 100% MeCN/water (0.1% TFA) in 10 min with UV detector (254 nm)) to afford 2-chloro-84 {4-[1-cyclopropyl-4-(trifluoromethyl)imidazo]-2-yl]phenyl}methyl)-6-(4-methylpiperazin-1-yl)pteridin-7-one (30 mg, 52.9%) as a yellow oil.

LC-MS (ESI) m/z 545.1 [M+H]

### Step 4: 8-(4-(1-Cyclopropyl-4-(Trifluoromethyl)-1H-Imidazol-2-Yl)Benzyl)-2-(4-Cyclopropyl-6-Methoxypyrimidin-5-Yl)-6-(4-Methylpiperazin-1-Yl)Pteridin-7(811)-One Formate

To a stirred solution of 2-chloro-8-({4-[1-cyclopropyl-4-(trifluoromethyl)imidazol-2-yl]phenyl}methyl)-6-(4-methylpiperazin-1-yl)pteridin-7-one (30 mg, mmol, 1 equiv) and 4-cyclopropyl-6-methoxypyrimidin-5-ylboronic acid (16.0 mg, 0.083 mmol, 1.5 equiv) in dioxane (1 mL) and H₂O (0.2 mL) were add Pd(dppf)Cl₂ (8.0 mg, 0.011 mmol, 0.2 equiv) and K₃PO₄ (29.2 mg, 0.138 mmol, 2.5 equiv) at room temperature. The resulting mixture was stirred for overnight at 90° C. under nitrogen atmosphere. The mixture was allowed to cool down to room temperature. The resulting mixture was filtered, the filter cake was washed with 1,4-dioxane (3×2 mL). The filtrate was concentrated under reduced pressure. The crude product was purified by Prep-HPLC with the following conditions (Column: Xselect CSH C18 OBD Column 30*150 mm 51 μm, n; Mobile Phase A: Water(0.1% FA), Mobile Phase B: MeOH-HPLC; Flow rate: 60 mL/min; Gradient: 40% B to 51% B in 10 min, 51% B; Wave Length: 254/220 nm; RT1(min): 8.67) to afford 8-(4-(1-cyclopropyl-4-(trifluoromethyl)-1H-imidazol-2-yl)benzyl)-2-(4-cyclopropyl-6-methoxypyrimidin-5-yl)-6-(4-methylpiperazin-1-yl)pteridin-7(8H)-one formate (13.5 mg, 33.4% yield, 98.1% purity) as an off-white solid.

LC-MS (ESI) m/z 659.3 [M+H].

¹H NMR (300 MHz, Methanol-d4) δ 8.89 (s, 1H), 8.62 (s, 1H), 7.82-7.70 (m, 3H), 7.59 (d, J=8.0 Hz, 2H), 5.67 (s, 2H), 4.27 (s, 4H), 3.93 (s, 3H), 3.64 (dd, J=7.1, 3.6 Hz, 1H), 2.98 (s, 4H), 2.62 (s, 3H), 1.83-1.70 (m, 1H), 1.14 (t, J=3.7 Hz, 2H), 1.03 (q, J=6.8, 6.3 Hz, 2H), 0.90-0.83 (m, 4H).

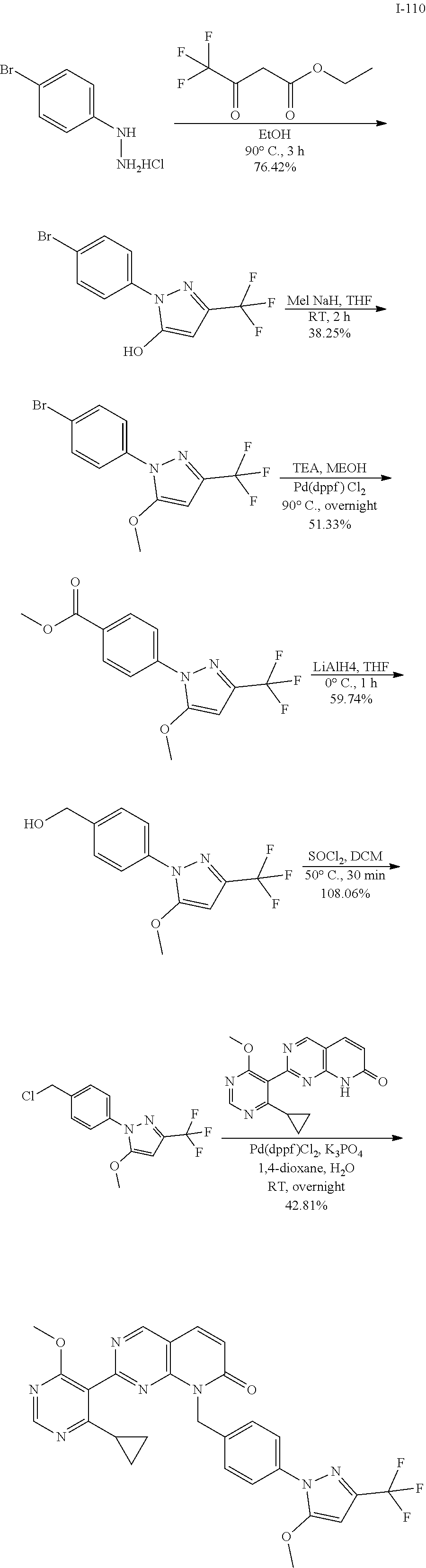

### Step 1: 2-(4-Bromophenyl)-5-(Trifluoromethyl)Pyrazol-3-Ol

To a stirred hydrazine, (4-bromophenyl)-hydrochloride (1 g, 4.474 mmol, 1 equiv) in ethyl alcohol (20 mL) was added ethyl 4,4,4-trifluoro-3-oxobutanoate (1.24 g, 6.711 mmol, 1.5 equiv) at room temperature under nitrogen atmosphere. The resulting mixture was stirred for 3h at 90° C. under nitrogen atmosphere. The mixture was allowed to cool down to room temperature. The residue was purified by reverse flash chromatography (C18 silica gel column, eluting with 0% to 100% MeCN/water (0.1% FA) in 10 min with UV detector (254 nm)) to afford 2-(4-bromophenyl)-5-(trifluoromethyl)pyrazol-3-ol (1.05 g, 76.42%) as an off-white solid.

LC-MS (ESI) m/z 307.1 [M+H]

### Step 2: 1-(4-Bromophenyl)-5-Methoxy-3-(Trifluoromethyl)Pyrazole

To a stirred 2-(4-bromophenyl)-5-(trifluoromethyl)pyrazol-3-ol (200 mg, 0.651 mmol, 1 equiv) in THF (4 mL, 20 equiv) was added NaH (23.4 mg, 0.977 mmol, 1.5 equiv, 60% in mineral oil) at 0° C. under nitrogen atmosphere. The resulting mixture was stirred for 30 min at under nitrogen atmosphere. To the above mixture was added methyl iodide (92.4 mg, 0.651 mmol, 1.0 equiv) at room temperature. The resulting mixture was stirred for additional 2h at room temperature. The reaction was quenched by the addition of Water (50 mL) at 0° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The mixture was allowed to cool down to room temperature. The residue was purified by reverse flash chromatography (C18 silica gel column, eluting with 0% to 100% MeCN/water (0.1% FA) in 10 min with UV detector (254 nm)) to afford 1-(4-bromophenyl)-5-methoxy-3-(trifluoromethyl)pyrazole (80 mg, 38.25%) as a yellow oil.

LC-MS (ESI) m/z 321.1 [M+H]

### Step 3. Methyl 4-[5-Methoxy-3-(Trifluoromethyl)Pyrazol-1-Yl]Benzoate

To a stirred 1-(4-bromophenyl)-5-methoxy-3-(trifluoromethyl)pyrazole (500 mg, 1.557 mmol, 1 equiv) in MeOH (10 mL, 20 equiv) were added Pd(dppf)Cl₂ (227.8 mg, 0.311 mmol, 0.2 equiv) and TEA (472.7 mg, 4.671 mmol, 3 equiv) at room temperature under carbon monoxide atmosphere. The resulting mixture was stirred for overnight at 90° C. under carbon monoxide atmosphere. The resulting mixture was filtered, the filter cake was washed with MeOH (3×30 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford methyl 4-[5-methoxy-3-(trifluoromethyl)pyrazol-1-yl]benzoate (240 mg, 51.33%) as a yellow oil.

LC-MS (ESI) m/z 301.1 [M+H]

### Step 4: {4-[5-Methoxy-3-(Trifluoromethyl)Pyrazol-1-Yl]Phenyl}Methanol

To a stirred methyl 4-[5-m ethoxy-3-(trifluoromethyl)pyrazol-1-yl]benzoate (240 mg, 0.799 mmol, 1 equiv) in THF (4.8 mL, 20 equiv) was added LiAlH₄ (91.0 mg, 2.397 mmol, 3.0 equiv) at 0° C. under nitrogen atmosphere. The resulting mixture was stirred for 1 h at under nitrogen atmosphere. The reaction was quenched with 10H2O.Na₂SO₄(aq) at 0° C. The precipitated solids were collected by filtration and washed with THF (3×3 mL). The resulting mixture was filtered, the filter cake was washed with THF (3×20 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford 445-m ethoxy-3-(trifluoromethyl)pyrazol-1-yl]phenyl 1methanol (130 mg, 59.74%) as a light yellow oil.

LC-MS (ESI) m/z 273.1 [M+H]

Step 5:

To a stirred {4-[5-methoxy-3-(trifluoromethyl)pyrazol-1-yl]phenyl}methanol (130 mg, 0.478 mmol, 1 equiv) in DCM (2.6 mL, 20 equiv) was added SOCl₂ (170.4 mg, 1.434 mmol, 3.0 equiv) at room temperature under nitrogen atmosphere. The resulting mixture was stirred for at 50° C. under nitrogen atmosphere. The reaction was quenched with Water at 0° C. The residue was neutralized to pH=7 with saturated NaHCO₃(aq.). The resulting mixture was extracted with CH₂Cl₂ (3×2 mL). The combined organic layers were washed with brine (2×4 mL), dried over anhydrous Na₂SO₄, filtered and concentrated under reduced pressure. The crude product was used in the next step directly without further purification.

LC-MS (ESI) m/z 291.1 [M+H]

### Step 6: 2-(4-Cyclopropyl-6-Methoxypyrimidin-5-Yl)-8-({4-[5-Methoxy-3-(Trifluoromethyl)Pyrazol-1-Yl]Phenyl}Methyl)Pyrido[2,3-d]Pyrimidin-7-One

To a stirred mixture of 1-[4-(chloromethyl)phenyl]-5-methoxy-3-(trifluoromethyl)pyrazole (50 mg, 0.172 mmol, 1 equiv) and 2-(4-cyclopropyl-6-methoxypyrimidin-5-yl)-8H-pyrido[2,3-d]pyrimidin-7-one (50.8 mg, 0.172 mmol, 1 equiv) in DMF (1 mL, 20 equiv) was added N,N,N′,N′-tetramethylguanidine (29.7 mg, 0.258 mmol, 1.5 equiv) at room temperature under nitrogen atmosphere. The resulting mixture was stirred for overnight at room temperature under nitrogen atmosphere. The resulting mixture was purified by Prep-HPLC with the following conditions (Column: Xselect CSH C18 OBD Column 51 μm, n; Mobile Phase A: Water(0.1% FA), Mobile Phase B: ACN; Flow rate: 60 mL/min; Gradient: 45% B to 57% B in 10 min; Wave Length: 254/220 nm; RT1(min): 8.83) to afford 2-(4-cyclopropyl-6-methoxypyrimidin-5-yl)-8-({4-[5-methoxy-3-(trifluoromethyl)pyrazol-1-yl]phenyl}methyl)pyrido[2,3-d]pyrimidin-7-one (42.6 mg, 42.81%, 95% purity) as a white solid.

LC-MS (ESI) m/z 550.15 [M+H]

¹H NMR (400 MHz, DMSO-d6) δ 9.29 (d, J=1.2 Hz, 1H), 8.69 (d, J=1.2 Hz, 1H), 8.16 (dd, J=9.5, 1.3 Hz, 1H), 7.57-7.50 (m, 2H), 7.41 (d, J=8.3 Hz, 2H), 6.90 (dd, J=9.5, 1.3 Hz, 1H), 6.45 (s, 1H), 5.55 (s, 2H), 3.96 (s, 3H), 3.82 (s, 3H), 1.69 (td, J=8.0, 4.0 Hz, 1H), 1.01 (dq, J=5.5, 3.5 Hz, 2H), 0.76 (dq, J=9.8, 3.4 Hz, 2H).

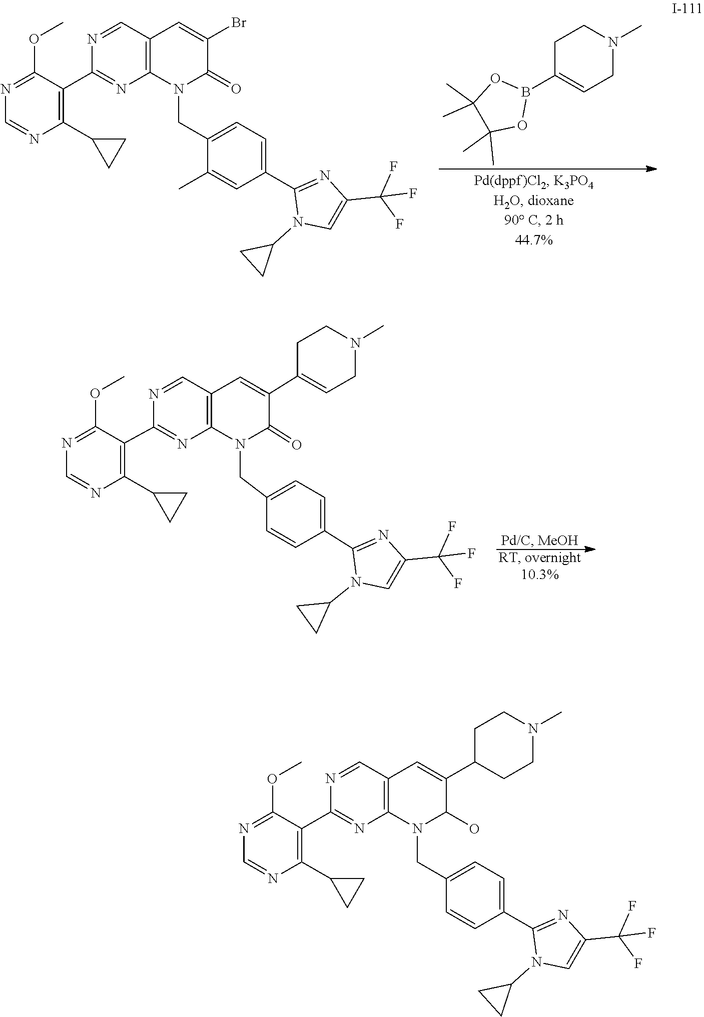

### Step 1 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(1-Methyl-3,6-Dihydro-211-Pyridin-4-Yl)Pyrido[2,3-d]Pyrimidin-7-One

To a stirred mixture of 6-bromo-8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (40 mg, 0.063 mmol, 1 equiv) and 1-methyl-4-(4,4,5,5-tetramethyl-1,3,2-dioxaborolan-2-yl)-3,6-dihydro-2H-pyridine (20.9 mg, 0.095 mmol, 1.5 equiv) in H₂O (0.2 mL, 11.102 mmol, 177.20 equiv) and dioxane (0.8 mL, 9.443 mmol, 150.72 equiv) were added K₃PO₄ (33.2 mg, 0.158 mmol, 2.5 equiv) and Pd(dppf)C12 (9.2 mg, 0.013 mmol, 0.2 equiv) at room temperature under nitrogen atmosphere. The resulting mixture was stirred for 2 h at 90° C. under nitrogen atmosphere. The mixture was allowed to cool down to room temperature. The resulting mixture was filtered, the filter cake was washed with acetonitrile (3×2 mL). The filtrate was concentrated under reduced pressure. The crude product was purified by Prep-HPLC with the following conditions (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 40% B to 58% B in 10 min, 58% B; Wave Length: 220/254 nm; RT1(min): 9.45) to afford 8-({4-[1-cyclopropyl-4-(trifluoromethyl)imidazo]-2-yl]phenyl methyl)-2-(4-cyclopropyl-6-methoxypyrimidin-5-yl)-6-(1-methyl-3,6-dihydro-2H-pyridin-4-yl)pyrido[2,3-d]pyrimidin-7-one (18.5 mg, 44.70%) as an off-white solid.

LC-MS (ESI) m/z 655.3 [M+H]

¹H NMR (400 MHz, DMSO-d6) δ 9.28 (s, 1H), 8.69 (s, 1H), 8.09 (s, 1H), 7.91 (s, J=1.4 Hz, 1H), 7.81 (d, J=8.3 Hz, 2H), 7.39 (d, J=8.2 Hz, 2H), 6.77 (s, 1H), 5.61 (s, 2H), 3.83 (s, 3H), 3.69 (m, J=7.2, 3.8 Hz, 1H), 3.06 (s, 2H), 2.57 (d, J=4.6 Hz, 2H), 2.50 (m, 2H), 2.28 (s, 3H), 1.79-1.69 (m, 1H), 1.06-0.92 (m, 4H), 0.93-0.86 (m, 2H), 0.77 (m, J=8.0, 3.4 Hz, 2H).

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(1-Methylpiperidin-4-Yl)Pyrido[2,3-d]Pyrimidin-7-One

To a solution of 8-({4-[1-cyclopropyl-4-(trifluoromethyl)imidazo]-2-yl]phenyl methyl)-2-(4-cyclopropyl-6-methoxypyrimidin-5-yl)-6-(1-methyl-3,6-dihydro-2H-pyridin-4-yl)pyrido[2,3-d]pyrimidin-7-one (50 mg, 0.076 mmol, 1 equiv) in 1 mL MeOH was added Pd/C (10%, 0.03 g) under nitrogen atmosphere. The mixture was hydrogenated at room temperature for overnight under hydrogen atmosphere, filtered through a Celite pad and concentrated under reduced pressure. The crude product was purified by Prep-HPLC with the following conditions (Column: YMC-Actus Triart C18 ExRS, 30*150 mm, 5 μm; Mobile Phase A: Water(10 mmol/L NH₄HCO₃), Mobile Phase B: ACN; Flow rate: 60 mL/min; Gradient: 43% B to 60% B in 9 min, 60% B; Wave Length: 254/220 nm; RT1(min): 8.65) to afford 8-({4-[1-cyclopropyl-4-(trifluoromethyl)imidazo]-2-yl]phenyl methyl)-2-(4-cyclopropyl-6-methoxypyrimidin-5-yl)-6-(1-methylpiperidin-4-yl)pyrido[2,3-d]pyrimidin-7-one (5.5 mg,94.6%, as an off-white solid.

LC-MS (ESI) m/z 657.3 [M+H]

¹H NMR (400 MHz, DMSO-d6) δ 9.26 (s, 1H), 8.69 (s, 1H), 7.99 (s, 1H), 7.91 (s, J=1.4 Hz, 1H), 7.80 (d, J=8.3 Hz, 2H), 7.40 (d, J=8.3 Hz, 2H), 5.60 (s, 2H), 3.83 (s, 3H), 3.73-3.64 (m, 1H), 2.94-2.85 (m, 2H), 2.83-2.72 (m, 1H), 2.20 (s, 3H), 1.98 (m, J=11.3 Hz, 2H), 1.83 (m, J=12.1 Hz, 2H), 1.72 (m, J=8.2, 4.0 Hz, 1H), 1.62 (m, J=13.9 Hz, 2H), 1.06-0.92 (m, 4H), 0.90 (m, 2H), 0.77 (m, J=8.0, 3.2 Hz, 2H).

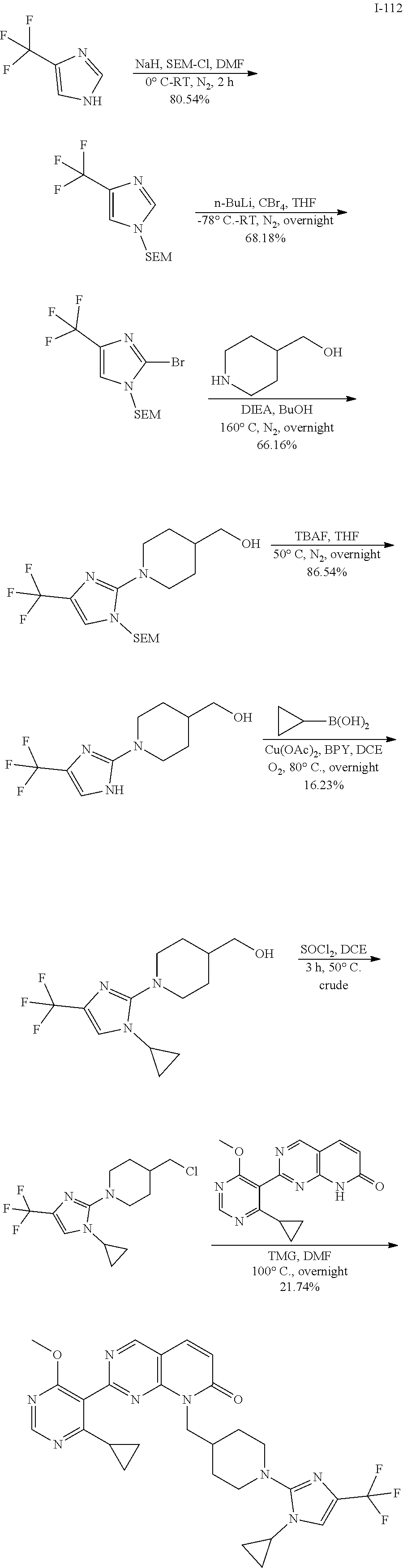

### Step 1. 4-(Trifluoromethyl)-1-{12-(Trimethylsilyl)Ethoxy]Methyl}Imidazole

To a stirred solution of 4-(trifluoromethyl)-1H-imidazole (5.5 g, 40.418 mmol, 1 equiv) in dimethylformamide (100 mL) was added NaH (3.23 g, 80.836 mmol, 2 equiv, 60% in mineral oil) in portions at 0° C. under nitrogen atmosphere. To the above mixture was added SEMCl (10.75 mL, 60.627 mmol, 1.5 equiv) dropwise at 0° C. The resulting mixture was stirred for additional 2 h at room temperature. The reaction was quenched with Water at room temperature. The aqueous layer was extracted with EtOAc (3×30 mL). The combined organic layers were washed with brine (2×50 mL), dried over anhydrous Na₂SO₄, filtered and concentrated under reduced pressure. The residue was purified by reversed-phase flash chromatography (C18 silica gel column, eluting with 0% to 100% MeCN/water (0.1% FA) in 10 min with UV detector (254 nm)) to afford 4-(trifluoromethyl)-1-{[2-(trimethylsilyl)ethoxy]methyl}imidazole (8.67 g, as a light yellow oil.

LC-MS (ESI) m/z 267.3 [M+H]

### Step 2. 2-Bromo-4-(Trifluoromethyl)-1-{12-(Trimethylsilyl)Ethoxy]Methyl}Imidazole

To a stirred solution of 4-(trifluoromethyl)-1-{[2-(trimethylsilyl)ethoxy]methyl}imidazole (4.3 g, 16.145 mmol, 1 equiv) in THF (40 mL) was added n-BuLi (11.7 mL, 124.196 mmol, 7.69 equiv) dropwise at −78° C. under nitrogen atmosphere. To the above mixture was added CBr₄ (8.03 g, 24.218 mmol, 1.5 equiv) at −78° C. The resulting mixture was stirred for additional overnight at room temperature. The reaction was quenched by the addition of Water (20 mL) at room temperature. The reaction was quenched by the addition of Water (20 mL) at room temperature. The resulting mixture was diluted with water (30 mL). The aqueous layer was extracted with EtOAc (3×40 mL). The combined organic layers were washed with brine (2×40 mL), dried over anhydrous Na₂SO₄, filtered and concentrated under reduced pressure. The residue was purified by reversed-phase flash chromatography (C18 silica gel column, eluting with 0% to 100% MeCN/water (0.1% FA) in 20 min with UV detector (254 nm)) to afford 2-bromo-4-(trifluoromethyl)-1-{[2-(tri methyl silyl)ethoxy]methyl}imidazole (3.8 g, 68.18%) as a yellow oil.

LC-MS (ESI) m/z 345.2 [M+H]

### Step 3: {1-[4-(Trifluoromethyl)-1-{12-(Trimethylsilyl)Ethoxy]Methyl}Imidazol-2-Yl]Piperidin-4-Yl}Methanol

To a stirred solution of 2-bromo-4-(trifluoromethyl)-1-{[2-(trimethylsilyl)ethoxy]methyl}imidazole (3.3 g, 9.559 mmol, 1 equiv) and piperidin-4-ylmethanol (5.50 g, 47.795 mmol, 5 equiv) in BuOH (17.47 mL, 191.180 mmol, 20 equiv) was added DIEA (3.71 g, 28.677 mmol, 3 equiv) at room temperature. The resulting mixture was stirred for overnight at 160° C. under nitrogen atmosphere. The mixture was purified by reversed-phase flash chromatography (C18 silica gel column, eluting with 0% to 100% MeCN/water (0.1% FA) in 10 min with UV detector (254 nm)) to afford {1-[4-(trifluoromethyl)-1-{[2-(trimethylsilyl)ethoxy]methyl}imidazol-2-yl]piperidin-4-yl}methanol (2.4 g, 66.16%) as a yellow oil.

LC-MS (ESI) m/z 380.5 [M+H]

### Step 4: {1-[4-(Trifluoromethyl)-1H-Imidazol-2-Yl]Piperidin-4-Yl}Methanol

To a stirred solution of {1-[4-(trifluoromethyl)-1-{[2-(trimethylsilyl)ethoxy]methyl}imidazol-2-yl]piperidin-4-yl}methanol (2.428 g, 6.398 mmol, 1 equiv) in THF (25 mL) was added TBAF (31.99 mL, 31.990 mmol, 5 equiv) dropwise at room temperature under nitrogen atmosphere. The resulting mixture was stirred for overnight at 50° C. under nitrogen atmosphere. The reaction was quenched with Water at room temperature. The resulting mixture was extracted with EtOAc (3×15 mL). The combined organic layers were washed with brine (2×20 mL), dried over anhydrous Na₂SO₄, filtered and concentrated under reduced pressure. The residue was purified by reversed-phase flash chromatography (C18 silica gel column, eluting with 0% to 100% MeCN/water (0.1% FA) in 10 min with UV detector (254 nm)) to afford {1-[4-(trifluoromethyl)-1H-imidazol-2-yl]piperidin-4-ylImethanol (1.38 g, 86.54%) as an off-white solid.

LC-MS (ESI) m/z 250.1 [M+H]

### Step 5: {1-[1-Cyclopropyl-4-(Trifluoromethyl)Imidazol-2-Yl]Piperidin-4-Yl}Methanol

To a stirred solution of {1-[4-(trifluoromethyl)-1H-imidazol-2-yl]piperidin-4-yl}methanol (1.38 g, 5.537 mmol, 1 equiv) and cyclopropylboronic acid (0.95 g, 11.074 mmol, 2 equiv) in DCE (20 mL) were added 2,2′-bipyridine (2.59 g, 16.611 mmol, 3 equiv) and Cu(OAc)₂ (3.02 g, 16.611 mmol, 3 equiv) at room temperature. The resulting mixture was stirred for overnight at 80° C. under Oxygen atmosphere. The resulting mixture was filtered, the filter cake was washed with DCM (3×10 mL). The filtrate was concentrated under reduced pressure. The residue was purified by silica gel column chromatography (eluting with PE/EA (5:1)) to afford {1-[1-cyclopropyl-4-(trifluoromethyl)imidazo]-2-yl]piperidin-4-yl}methanol (260 mg, 16.23%) as a light yellow solid.

LC-MS (ESI) m/z 290.3 [M+H]

### Step 6 4-(Chloromethyl)-1-[1-Cyclopropyl-4-(Trifluoromethyl)Imidazol-2-Yl]Piperidine

To a stirred solution of {1-[1-cyclopropyl-4-(trifluoromethyl)imidazo]-2-yl]piperidin-4-yl}methanol (173 mg, 0.598 mmol, 1 equiv) in DCE (3 mL) was added SOCl₂ (0.22 mL, 2.990 mmol, 5 equiv) at room temperature. The resulting mixture was stirred for 3 h at 50° C. under nitrogen atmosphere. The reaction was quenched with Water at room temperature. The resulting mixture was extracted with EtOAc (3×15 mL). The combined organic layers were washed with brine (2×20 mL), dried over anhydrous Na₂SO₄, filtered and concentrated under reduced pressure to afford 4-(chloromethyl)-1-[1-cyclopropyl-4-(trifluoromethyl)imidazol-2-yl]piperidine (180 mg, crude) as a yellow oil.

LC-MS (ESI) m/z 308.1 [M+H]

### Step 7: 8-({1-[1-Cyclopropyl-4-(Trifluoromethyl)Imidazol-2-Yl]Piperidin-4-Yl}Methyl)-2-(4-Cyclopropyl-6-Methoxypyrimidin-5-Yl)Pyrido[2,3-d]Pyrimidin-7-One

To a stirred solution of 4-(chloromethyl)-1-[1-cyclopropyl-4-(trifluoromethyl)imidazol-2-yl]piperidine (30 mg, 0.097 mmol, 1 equiv) and 2-(4-cyclopropyl-6-methoxypyrimidin-5-yl)-8H-pyrido[2,3-d]pyrimidin-7-one (28.8 mg, 0.097 mmol, 1 equiv) in DMF (1 mL) was added 1,1,3,3-tetramethylguanidine (16.9 mg, 0.146 mmol, 1.5 equiv) at room temperature. The resulting mixture was stirred for overnight at 100° C. under nitrogen atmosphere. The residue was purified by reversed-phase flash chromatography (C18 silica gel column, eluting with 0% to 100% MeCN/water (0.1% FA) in 10 min with UV detector (254 nm)) to the crude product. The crude product (40 mg) was purified by Prep-HPLC with the following conditions (Column: XBridge Prep C18 OBD Column, 30*100 mm, 51 μm; Mobile Phase A: Water(0.05% TFA), Mobile Phase B: ACN; Flow rate: 60 mL/min; Gradient: 38% B to 52% B in min, 52% B; Wave Length: 220/254 nm; RT1(min): 10.23) to afford 8-({1-[1-cyclopropyl-4-(trifluoromethyl)imidazo]-2-yl]piperidin-4-yl}methyl)-2-(4-cyclopropyl-6-methoxypyrimidin-5-yl)pyrido[2,3-d]pyrimidin-7-one (12.3 mg, 22.7%) as an off-white solid.

LC-MS (ESI) m/z 567.3 [M+H]

¹H NMR: (400 MHz, DMSO-d6) δ 9.24 (s, 1H), 8.71 (s, 1H), 8.10 (d, J=9.5 Hz, 1H), 7.42 (d, J=1.6 Hz, 1H), 6.85 (d, J=9.5 Hz, 1H), 4.29 (d, J=7.1 Hz, 2H), 3.86 (s, 3H), 3.51 (d, J=12.2 Hz, 2H), 3.29 (d, J=5.3 Hz, 1H), 2.70-2.59 (m, 2H), 2.05 (t, J=9.4 Hz, 1H), 1.81 (td, J=8.2, 4.2 Hz, 1H), 1.61 (d, J=12.7 Hz, 2H), 1.52-1.42 (m, 2H), 1.08 (h, J=3.5 Hz, 2H), (d, J=5.6 Hz, 4H), 0.90 (dq, J=7.0, 3.4 Hz, 2H).

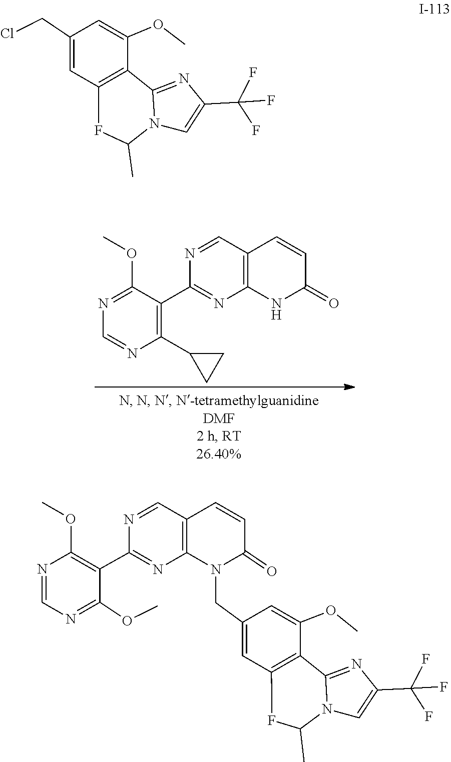

### Step 1: 2-(4,6-Dimethoxypyrimidin-5-Yl)-8-({3-Fluoro-4-[1-Isopropyl-4-(Trifluoromethyl)Imidazol-2-Yl]-5-Methoxyphenyl}Methyl)Pyrido[2,3-d]Pyrimidin-7-One

To a stirred mixture of 2-[4-(chloromethyl)-2-fluoro-6-methoxyphenyl]-1-isopropyl-4-(trifluoromethyl)imidazole (40 mg, 0.114 mmol, 1 equiv) and 2-(4,6-dimethoxypyrimidin-5-yl)-8H-pyrido[2,3-d]pyrimidin-7-one (39.0 mg, 0.137 mmol, 1.2 equiv) in DMF (1 mL, 12.922 mmol, 113.30 equiv) was added N,N,N′,N′-tetramethylguanidine (19.7 mg, 0.171 mmol, 1.50 equiv) at room temperature under nitrogen atmosphere. The resulting mixture was stirred for additional 2 h at room temperature. The resulting mixture was purified by reversed-phase flash chromatography (C18 silica gel column, eluting with 0% to 100% MeCN/water (0.1% FA) in 10 min with UV detector (254 nm)) to afford 244,6-dimethoxypyrimidin-5-yl)-8-({3-fluoro-4-[1-isopropyl-4-(trifluoromethyl)imidazol-2-yl]-5-methoxyphenyl}methyl)pyrido[2,3-d]pyrimidin-7-one (19 mg, 26.40%) as a white solid.

LC-MS (ESI) m/z 600.05 [M+H\

¹H NMR (400 MHz, DMSO-d6) δ 9.24 (s, 1H), 8.63 (s, 1H), 8.22-8.03 (m, 2H), 7.06 (s, 1H), 6.89 (d, J=9.6 Hz, 1H), 6.78 (d, J=9.6 Hz, 1H), 5.52 (s, 2H), 3.92 (h, J=6.7 Hz, 1H), 3.84 (s, 6H), 3.71 (s, 3H), 1.35 (d, J=6.6 Hz, 3H), 1.23 (d, J=6.7 Hz, 3H).

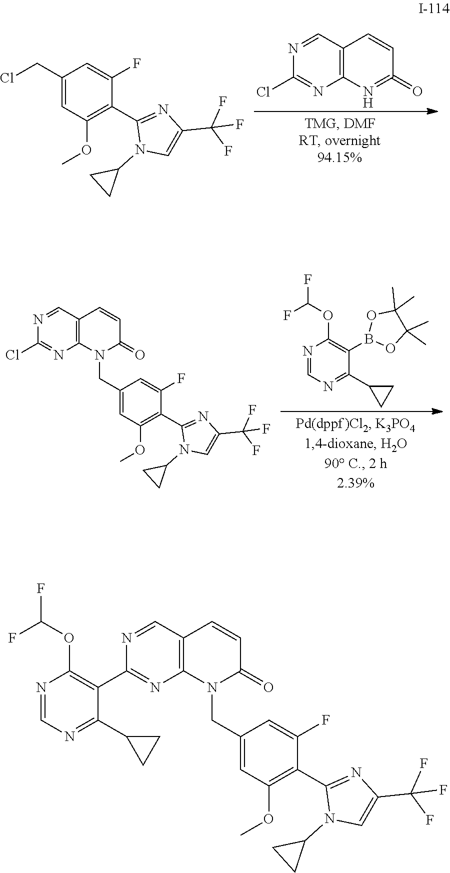

### Step 1 2-Chloro-8-({4-[1-Cyclopropyl-4-(Trifluoromethyl)Imidazol-2-Yl]-3-Fluoro-5-Methoxyphenyl}Methyl)Pyrido[2,3-d]Pyrimidin-7-One

To a stirred mixture of 2-[4-(chloromethyl)-2-fluoro-6-methoxyphenyl]-1-cyclopropyl-4-(trifluoromethyl)imidazole (60 mg, 0.172 mmol, 1 equiv) and 2-chloro-8H-pyrido[2,3-d]pyrimidin-7-one (31.2 mg, 0.172 mmol, 1 equiv) in DMF (1.2 mL, 3.440 mmol, 20 equiv) was added N,N,N′,N′-tetramethylguanidine (29.7 mg, 0.258 mmol, 1.5 equiv) at room temperature under nitrogen atmosphere. The resulting mixture was stirred for overnight at room temperature under nitrogen atmosphere. The resulting mixture was purified by reverse flash chromatography (C18 silica gel column, eluting with 0% to 100% MeCN/water (0.1% FA) in 10 min with UV detector (254 nm)) to afford 2-chloro-8-({4-[1-cyclopropyl-4-(trifluoromethyl)imidazol-2-yl]-3-fluoro-5-methoxyphenyl methyl)pyrido[2,3-d]pyrimidin-7-one (80 mg, 94.15%) as a brown oil.

LC-MS (ESI) m/z 494.10 [M+H]

### Step 2: 8-({4-[1-Cyclopropyl-4-(Trifluoromethyl)Imidazol-2-Yl]-3-Fluoro-5-Methoxyphenyl}Methyl)-2-[4-Cyclopropyl-6-(Difluoromethoxy)Pyrimidin-5-Yl]Pyrido[2,3-d]Pyrimidin-7-One

To a stirred mixture of 2-chloro-8-({4-[1-cyclopropyl-4-(trifluoromethyl)imidazo]-2-yl]-3-fluoro-5-methoxyphenyl}methyl)pyrido[2,3-d]pyrimidin-7-one (80 mg, 0.162 mmol, 1 equiv) and 4-cyclopropyl-6-(difluoromethoxy)-5-(4,4,5,5-tetramethyl-1,3,2-dioxaborolan-2-yl)pyrimidine (75.8 mg, 0.243 mmol, 1.5 equiv) in 1,4-dioxane (1.6 mL, 20 equiv) and H₂O (0.4 mL, 0.5 equiv) were added Pd(dppf)C12 (11.8 mg, 0.016 mmol, 0.1 equiv) and K₃PO₄ (103.1 mg, mmol, 3.0 equiv) at room temperature under nitrogen atmosphere. The resulting mixture was stirred for 2 h at 90° C. under nitrogen atmosphere. The mixture was allowed to cool down to room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was dissolved in DMF (1 mL) and purified by Prep-HPLC with the following conditions (Column: XBridge Prep Phenyl OBD Column, 19*250 mm, 51 μm; Mobile Phase A: Water(0.1% FA), Mobile Phase B: ACN; Flow rate: mL/min; Gradient: 47% B to 63% B in 8 min; Wave Length: 254/220 nm; RT1(min): 7.71) to afford 8-({4-[1-cyclopropyl-4-(trifluoromethyl)imidazol-2-yl]-3-fluoro-5-methoxyphenyl methyl)-2-[4-cyclopropyl-6-(difluoromethoxy)pyrimidin-5-yl]pyrido[2,3-d]pyrimidin-7-one (2.5 mg, 2.39%) as a white solid.

LC-MS (ESI) m/z 644.25 [M+H]

¹H NMR (400 MHz, DMSO-d6) δ 9.35 (s, 1H), 8.82 (s, 1H), 8.20 (d, J=9.5 Hz, 1H), 8.00-7.59 (m, 2H), 7.02 (d, J=6.7 Hz, 1H), 6.94 (d, J=9.5 Hz, 1H), 6.71 (dd, J=9.5, 5.7 Hz, 1H), 5.57 (d, J=6.6 Hz, 2H), 3.70 (s, 4H), 4.22-3.64 (m, 1H), 3.17 (p, J=6.5 Hz, 1H), 1.93 (tt, J=7.6, 4.3 Hz, 1H), 1.08 (m, 2H), 0.98-0.62 (m, 5H).

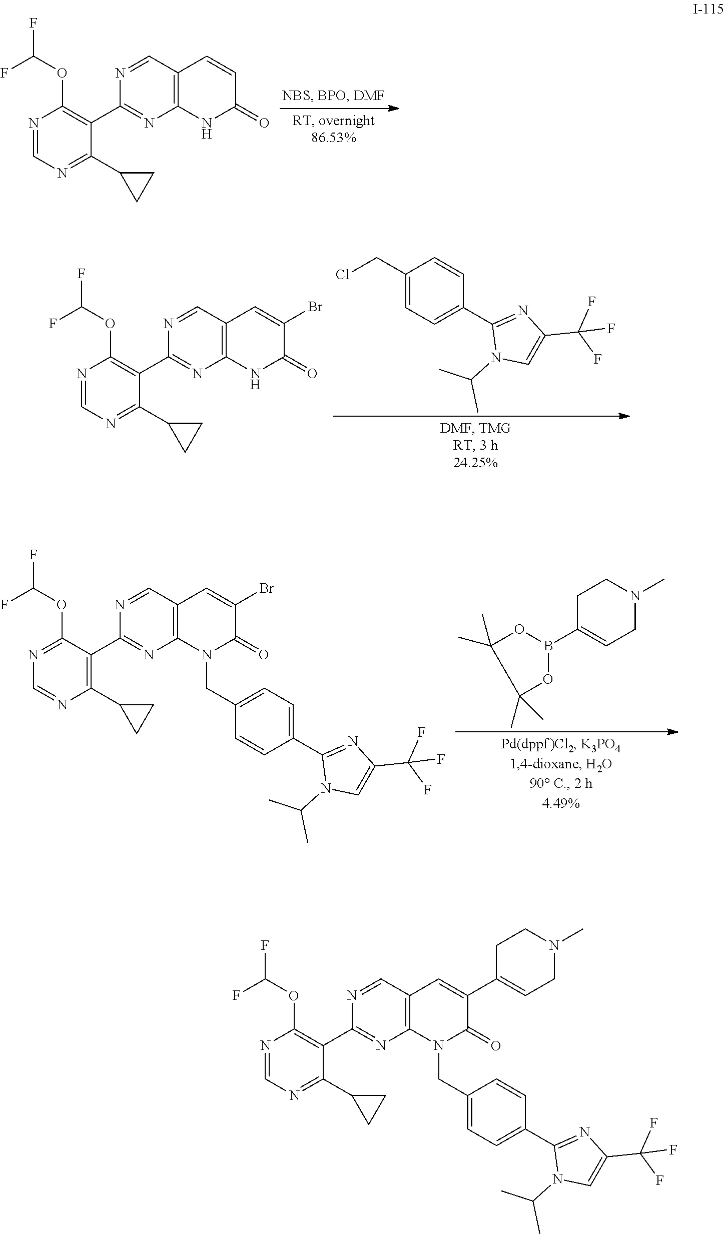

Step 1: 6-bromo-2-[4-cyclopropyl-6-(difluoromethoxy)pyrimidin-5-yl]-811-pyrido[2,3-d]pyrimidin-7-one To a stirred mixture of 2-[4-cyclopropyl-6-(difluoromethoxy)pyrimidin-5-yl]-8H-pyrido[2,3-d]pyrimidin-7-one (70 mg, 0.211 mmol, 1 equiv) in DMF (1.4 mL, 4.220 mmol, 20 equiv) were added NB S (112.8 mg, 0.633 mmol, 3.0 equiv) and BPO (5.4 mg, 0.021 mmol, 0.1 equiv) at room temperature under nitrogen atmosphere. The resulting mixture was stirred for overnight at room temperature under nitrogen atmosphere. The resulting mixture was purified by reverse flash chromatography (C18 silica gel column, eluting with 0% to 100% MeCN/water (0.1% FA) in 10 min with UV detector (254 nm)) to afford 6-bromo-2-[4-cyclopropyl-6-(difluoromethoxy)pyrimidin-5-yl]-8H-pyrido[2,3-d]pyrimidin-7-one (75 mg, 86.53%) as a yellow solid.

LC-MS (ESI) m/z 410.1 [M+H]

### Step 2: 6-Bromo-2-[4-Cyclopropyl-6-(Difluoromethoxy)Pyrimidin-5-Yl]-8-({4-[1-Isopropyl-4-(Trifluoromethyl)Imidazol-2-Yl]Phenyl}Methyl)Pyrido[2,3-d]Pyrimidin-7-One

To a stirred mixture of 6-bromo-2-[4-cyclopropyl-6-(difluoromethoxy)pyrimidin-(75 mg, 0.183 mmol, 1.00 equiv) and 2-[4-(chloromethyl)phenyl]-1-isopropyl-4-(trifluoromethyl)imidazole (44.2 mg, 0.146 mmol, 0.8 equiv) in DMF (1.5 mL, 20 equiv) was added N,N,N′,N′-tetramethylguanidine (31.5 mg, 0.274 mmol, 1.5 equiv) at room temperature under nitrogen atmosphere. The resulting mixture was stirred for 3h at room temperature under nitrogen atmosphere. The resulting mixture was purified by reverse flash chromatography (C18 silica gel column, eluting with 0% to 100% MeCN/water (0.1% FA) in 10 min with UV detector (254 nm)) to afford 6-bromo-2-[4-cyclopropyl-6-(difluoromethoxy)pyrimidin-5-yl]-8-({4-[1-isopropyl-4-(trifluoromethyl)imidazol-2-yl]phenyl}methyl)pyrido[2,3-d]pyrimidin-7-one (30 mg, 24.25%) as a light yellow solid.

LC-MS (ESI) m/z 678.2 [M+H]

### Step 3: 2-[4-Cyclopropyl-6-(Difluoromethoxy)Pyrimidin-5-Yl]-8-({4-[1-Isopropyl-4-(Trifluoromethyl)Imidazol-2-Yl]Phenyl}Methyl)-6-(1-Methyl-3,6-Dihydro-211-Pyridin-4-Yl)Pyrido[2,3-d]Pyrimidin-7-One

To a stirred mixture of 6-bromo-2-[4-cyclopropyl-6-(difluoromethoxy)pyrimidin-441-isopropyl-4-(trifluoromethyl)imidazol-2-yl]phenyl}methyl)pyrido[2,3-d]pyrimidin-7-one (30 mg, 0.044 mmol, 1 equiv) and 1-methyl-4-(4,4,5,5-tetramethyl-1,3,2-dioxaborolan-2-yl)-3,6-dihydro-2H-pyridine (14.8 mg, 0.066 mmol, 1.5 equiv) in H₂O (0.12 mL, 1.184 mmol, 4 equiv) and 1,4-dioxane (0.6 mL, 20 equiv) were added Pd(dppf)Cl₂ (3.2 mg, 0.004 mmol, 0.1 equiv) and K₃PO₄ (28.2 mg, 0.132 mmol, 3.0 equiv) at room temperature under nitrogen atmosphere. The resulting mixture was stirred for 2 h at under nitrogen atmosphere. The mixture was allowed to cool down to room temperature. The resulting mixture was extracted with EtOAc (3×3 mL). The combined organic layers were washed with brine (2×3 mL), dried over anhydrous Na₂SO₄, filtered and concentrated under reduced pressure. The crude product (30 mg) was purified by Prep-HPLC with the following conditions (Column: XBridge Shield RP18 OBD Column, 30*150 mm, 51 μm; Mobile Phase A: Water(10 mmol/L FA), Mobile Phase B: ACN; Flow rate: 60 mL/min; Gradient: 33% B to 50% B in 10 min, 50% B; Wave Length: 254/220 nm; RT1(min): 9.13) to afford 2-[4-cyclopropyl-6-(difluoromethoxy)pyrimidin-5-yl]-8-({4-[1-isopropyl-4-(trifluoromethyl)imidazol-2-yl]phenyl}methyl)-6-(1-methyl-3, 6-dihydro-2H-pyridin-4-yl)pyrido[2,3-d]pyrimidin-7-one (1.4 mg, 4.49%) as an off-white solid.

LC-MS (ESI) m/z 693.0 [M+H]

¹H NMR (400 MHz, DMSO-d6) δ 9.33 (s, 1H), 8.82 (s, 1H), 8.17-8.10 (m, 2H), 7.82 (t, J=71.5 Hz, 1H), 7.47-7.41 (m, 4H), 6.78 (m, 1H), 5.62 (s, 2H), 4.41 (h, J=6.6 Hz, 1H), 3.06 (s, 2H), 2.57 (d, J=4.2 Hz, 4H), 2.28 (s, 3H), 1.85 (m, 1H), 1.37 (d, J=6.7 Hz, 6H), 1.08 (m, 2H), 0.84 (dd, J=8.0, 3.3 Hz, 2H).

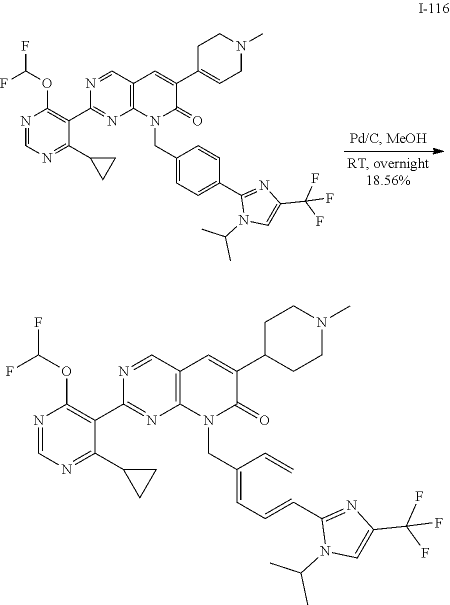 [SMILES: CC(C)n1cc(C(F)(F)F)nc1-c1ccc(Cn2c(=O)c(C3=CCN(C)CC3)cc3cnc(-c4c(OC(F)F)ncnc4C4CC4)nc32)cc1 ; C=C/C(=C\C=C\c1nc(C(F)(F)F)cn1C(C)C)Cn1c(=O)c(C2CCN(C)CC2)cc2cnc(-c3c(OC(F)F)ncnc3C3CC3)nc21]

To a stirred solution of 2-[4-cyclopropyl-6-(difluoromethoxy)pyrimidin-5-yl]-8-({4-[1-isopropyl-4-(trifluoromethyl)imidazol-2-yl]phenyl methyl)-6-(1-methyl-3,6-dihydro-2H-pyridin-4-yl)pyrido[2,3-d]pyrimidin-7-one (110 mg, 0.159 mmol, 1 equiv) in MeOH (2.2 mL, 20 equiv) was added Pd/C (55 mg, 20% wt) at room temperature under hydrogen atmosphere. The resulting mixture was stirred for overnight at room temperature under hydrogen atmosphere. The resulting mixture was filtered through a Celite pad, the filter cake was washed with MeOH (2×2 mL). The filtrate was purified by Prep-HPLC with the following conditions (Column: XBridge Shield RP18 OBD Column, 30*150 mm, 51 μm; Mobile Phase A: Water(10 mmol/L NH 4 HCO 3), Mobile Phase B: ACN; Flow rate: 60 mL/min; Gradient: 43% B to 60% B in 10 min, 60% B; Wave Length: 254/220 nm; RT1(min): 9.13) to afford 2-[4-cyclopropyl-6-(difluoromethoxy)pyrimidin-441-isopropyl-4-(trifluoromethyl)imidazol-2-yl]phenyl methyl)-6-(1-methylpiperidin-4-yl)pyrido[2,3-d]pyrimidin-7-one (21 mg, 18.56%,purity:97.5%) as an off-white solid.

LC-MS (ESI) m/z 695.45 [M+H]

¹H NMR (400 MHz, DMSO-d6) δ 9.31 (s, 1H), 8.82 (s, 1H), 8.16 (d, J=1.4 Hz, 1H), 8.02 (d, J=0.9 Hz, 1H), 7.81 (t, J=71.5 Hz, 1H), 7.49-7.39 (m, 4H), 5.61 (s, 2H), 4.41 (h, J=6.6 Hz, 1H), 2.90 (d, J=11.2 Hz, 2H), 2.78 (t, J=12.0 Hz, 1H), 2.21 (s, 3H), 2.03-1.92 (m, 2H), 1.85 (dd, J=10.1, 6.2 Hz, 3H), 1.63 (td, J=12.2, 3.6 Hz, 2H), 1.37 (d, J=6.6 Hz, 6H), 1.08 (q, J=3.8 Hz, 2H), 0.84 (dq, J=7.1, 3.6 Hz, 2H).

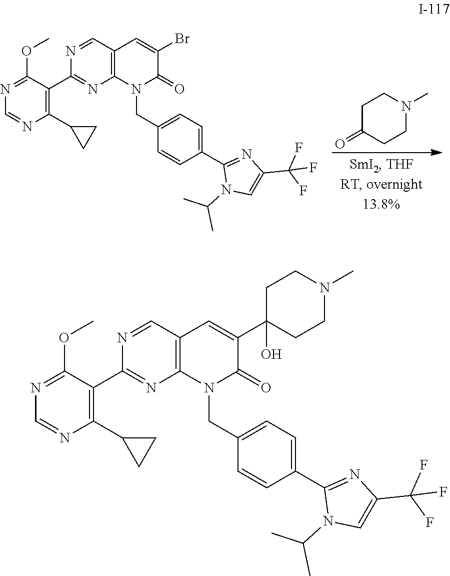

A solution of 6-bromo-2-(4-cyclopropyl-6-methoxypyrimidin-5-yl)-8-({4-[1-isopropyl-4-(trifluoromethyl)imidazo]-2-yl]phenyl methyl)pyrido[2,3-d]pyrimidin-7-one (100 mg, 0.156 mmol, 1 equiv) and 1-methylpiperidin-4-one (35.3 mg, 0.312 mmol, 2 equiv) in THF (2 mL) was treated with SmI₂ (10.93 mL, 1.092 mmol, 7 equiv) for 4 h at −78° C. under nitrogen atmosphere followed by the addition of SmI₂ (0.62 mL, 0.062 mmol, 0.4 equiv) dropwise at −78° C. The resulting mixture was stirred for overnight at room temperature under nitrogen atmosphere. The reaction was quenched by the addition of sat. NH₄Cl (aq.) (50 mL) at 0° C. The resulting mixture was extracted with EtOAc (3×50 mL). The combined organic layers were washed with brine (3×50 mL), dried over anhydrous Na₂SO₄, filtered and concentrated under reduced pressure. The crude product was purified by Prep-HPLC with the following conditions (Column: YMC-Actus Triart C18 ExRS, 30*150 mm, 5 μm; Mobile Phase A: Water(10 mmol/L NH₄HCO₃), Mobile Phase B: ACN; Flow rate: 60 mL/min; Gradient: 35% B to 52% B in 9 min, 52% B; Wave Length: 254/220 nm; RT1(min): 10.38) to afford 2-(4-cyclopropyl-6-methoxypyrimidin-5-yl)-6-(4-hydroxy-1-methylpiperidin-4-yl)-8-({4-[1-isopropyl-4-(trifluoromethyl)imidazo]-2-yl]phenyl}methyl)pyrido[2,3-d]pyrimidin-7-one (14.9 mg, 13.87%) as an off-white solid.

LC-MS (ESI) m/z 675.3 [M+H]

¹H NMR (400 MHz, DMSO-d6) δ 9.36 (s, 1H), 8.69 (s, 1H), 8.34 (s, 1H), 8.16 (s, J=1.4 Hz, 1H), 7.49 (d, J=8.2 Hz, 2H), 7.43 (d, J=8.1 Hz, 2H), 5.60 (s, 2H), 5.20 (s, 1H), 4.42 (h, J=6.6 Hz, 1H), 3.83 (s, 3H), 2.61 (m, J=11.8 Hz, 4H), 2.45 (d, J=11.9 Hz, 2H), 2.26 (s, 3H), 1.72 (m, J=8.2, 4.6 Hz, 1H), 1.47-1.31 (m, 8H), 1.01 (m, J=3.5 Hz, 2H), 0.76 (m, J=6.9, 3.4 Hz, 2H).

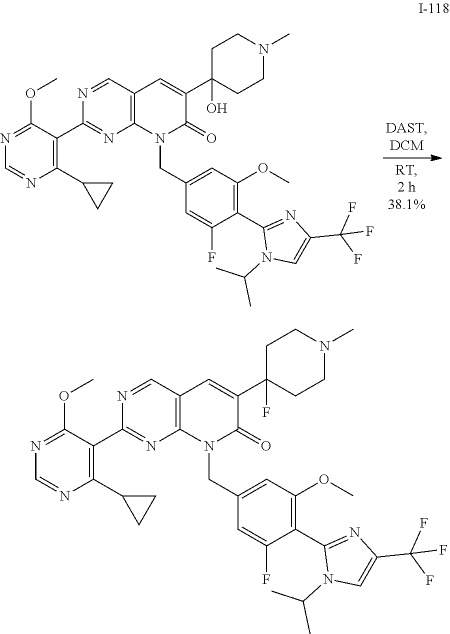

To a stirred mixture of 2-(4-cyclopropyl-6-methoxypyrimidin-5-yl)-8-({4-[1-ethyl-4-(trifluoromethyl)imidazo]-2-yl]-3-fluoro-5-methoxyphenyl}methyl)-6-(4-hydroxy-1-methylpiperidin-4-yl)pyrido[2,3-d]pyrimidin-7-one (40 mg, 0.056 mmol, 1 equiv) in DCM (1.5 mL, 23.596 mmol, 418.07 equiv) was added DAST (54.6 mg, 0.336 mmol, 6 equiv) dropwise at under nitrogen atmosphere. The resulting mixture was stirred for 2 h at room temperature under nitrogen atmosphere. The reaction was quenched by the addition of sat. NH₄Cl (aq.) (10 mL) at 0° C. The resulting mixture was extracted with CH₂Cl₂ (3×10 mL). The combined organic layers were washed with brine (3×10 mL), dried over anhydrous Na₂SO₄, filtered and concentrated under reduced pressure. The crude product (50 mg) was purified by Prep-HPLC with the following conditions (Column: XBridge Prep Phenyl OBD Column, 19*250 mm, 5 μm; Mobile Phase A: Water(10 mmol/L NH₄HCO₃), Mobile Phase B: ACN; Flow rate: 50 mL/min; Gradient: 57% B to 74% B in 8 min, 74% B; Wave Length: 254/220 nm; RT1(min): 6.41;) to afford 2-(4-cyclopropyl-6-methoxypyrimidin-5-yl)-6-(4-fluoro-1-methylpiperidin-4-yl)-8-({3-fluoro-4-[1-isopropyl-4-(trifluoromethyl)imidazol-2-yl]-5-methoxyphenyl}methyl)pyrido[2,3-d]pyrimidin-7-one (15.7 mg, 38.11%) as an off-white solid.

LC-MS (ESI) m/z 725.3 [M+H]

¹H NMR (400 MHz, DMSO-d6) δ 9.40 (s, 1H), 8.69 (s, 1H), 8.28 (s, 1H), 8.17 (d, J=1.3 Hz, 1H), 7.06 (s, 1H), 6.75 (d, J=9.7 Hz, 1H), 5.58 (m, J=15.1 Hz, 2H), 3.92 (h, J=6.6 Hz, 1H), 3.81 (s, 3H), 3.69 (s, 3H), 2.68 (m, 4H), 2.23 (m, 5H), 1.74 (m, J=8.5, 4.0 Hz, 3H), 1.35 (d, J=6.6 Hz, 3H), 1.23 (d, J=6.6 Hz, 3H), 1.08-0.96 (m, 2H), 0.78 (d, J=7.6 Hz, 2H).

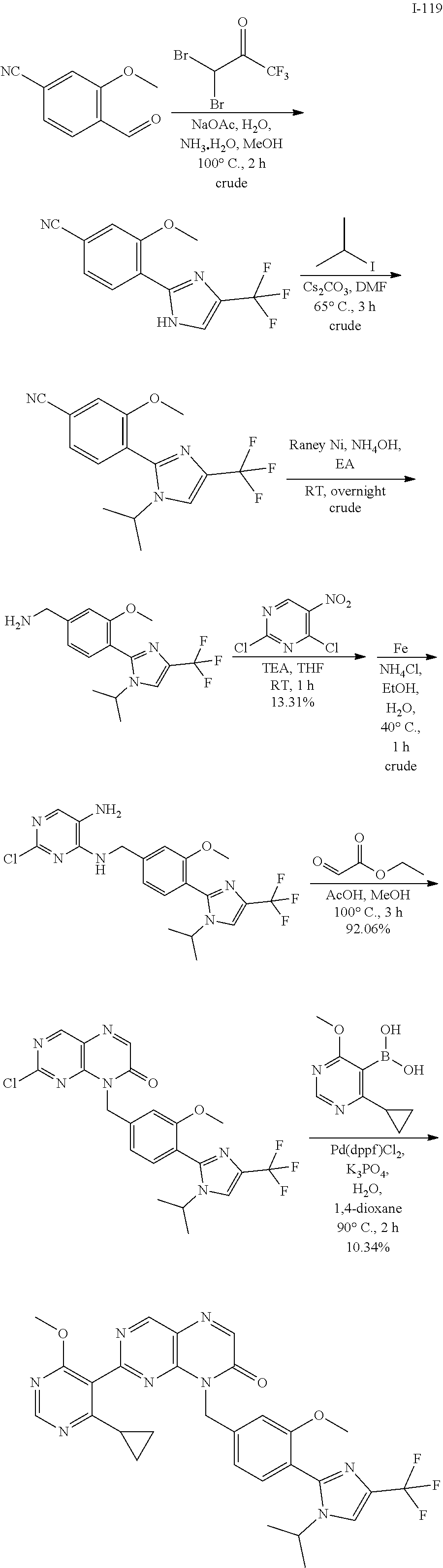

### Step 1: 3-Methoxy-4-[4-(Trifluoromethyl)-1H-Imidazol-2-Yl]Benzonitrile

A solution of 3,3-dibromo-1,1,1-trifluoropropan-2-one (3.78 g, 14.023 mmol, 1.13 equiv) and NaOAc (1.15 g, 14.023 mmol, 1.13 equiv) in H₂O (4 mL) was stirred for 1 h at 100° C. under nitrogen atmosphere. The mixture was allowed to cool down to room temperature. To the above mixture was added 4-formyl-3-methoxybenzonitrile (2 g, 12.410 mmol, 1 equiv), NH₃.H₂O (1.55 g, 44.180 mmol, 3.56 equiv) and MeOH (30.4 mL) at room temperature. The resulting mixture was stirred for additional 50 min at room temperature. The resulting mixture was stirred for 2 h at 100° C. under nitrogen atmosphere. The mixture was allowed to cool down to room temperature. The resulting mixture was diluted with water (200 mL). The resulting mixture was extracted with EtOAc (3×500 mL). The combined organic layers were washed with brine (2×500 mL), dried over anhydrous Na₂SO₄, filtered and was concentrated under reduced pressure to afford 3-methoxy-4-[4-(trifluoromethyl)-1H-imidazol-2-yl]benzonitrile (2.27 g, crude) as a yellow solid.

LC-MS (ESI) m/z 268.10[M+H]

Step 2:

To a stirred solution of 3-methoxy-4-[4-(trifluoromethyl)-1H-imidazol-2-yl]benzonitrile (200 mg, 0.748 mmol, 1 equiv) in DMF (4 mL) were added 2-iodopropane (381.71 mg, 2.244 mmol, 3.0 equiv) and Cs₂CO₃ (487.73 mg, 1.496 mmol, 2.0 equiv) at room temperature under nitrogen atmosphere. The resulting mixture was stirred for 3h at under nitrogen atmosphere. The mixture was allowed to cool down to room temperature. The reaction was quenched by the addition of Water (20 mL) at 0° C. The resulting mixture was extracted with EtOAc (3×20 mL). The combined organic layers were washed with brine (2×20 mL), dried over anhydrous Na₂SO₄, filtered and concentrated under reduced pressure to afford 4-[1-isopropyl-4-(trifluoromethyl)imidazol-2-yl]-3-methoxybenzonitrile (330 mg, crude) as a yellow oil. The crude product was used in the next step directly without further purification.

LC-MS (ESI) m/z 310.10[M+H]

Step 3.

To a stirred mixture of 4-[1-isopropyl-4-(trifluoromethyl)imidazol-2-yl]-3-methoxybenzonitrile (330 mg, 1.067 mmol, 1 equiv) in EA (4 mL) were added NH 4 OH (4 mL) and Raney Ni (500 mg) at room temperature under hydrogen atmosphere. The resulting mixture was stirred for overnight at room temperature under hydrogen atmosphere. The resulting mixture was filtered, the filter cake was washed with EtOAc (3×20 mL). The filtrate was concentrated under reduced pressure to afford 1-{4-[1-isopropyl-4-(trifluoromethyl)imidazo]-2-yl]-3-methoxyphenyl}methanamine (700 mg, crude) as a yellow solid. The crude product was used in the next step directly without further purification.

LC-MS (ESI) m/z 314.10[M+H]

### Step 4: 2-Chloro-N-({4-[1-Isopropyl-4-(Trifluoromethyl)Imidazol-2-Yl]-3-Methoxyphenyl}Methyl)-5-Nitropyrimidin-4-Amine

A solution of 1-{4-[1-isopropyl-4-(trifluoromethyl)imidazol-2-yl]-3-methoxyphenyl}methanamine (300 mg, 0.957 mmol, 1 equiv) in THF (6 mL) was treated with 2,4-dichloro-5-nitropyrimidine (371.44 mg, 1.914 mmol, 2 equiv) at room temperature under nitrogen atmosphere followed by the addition of TEA (290.67 mg, 2.871 mmol, 3 equiv) dropwise at 0° C. The resulting mixture was stirred for 1 h at room temperature under nitrogen atmosphere. The resulting mixture was diluted with water (50 mL). The resulting mixture was extracted with EtOAc (3×50 mL). The combined organic layers were washed with brine (2×50 mL), dried over anhydrous Na₂SO₄, filtered and concentrated under reduced pressure. The residue was purified by silica gel column chromatography, eluted with PE/EA (1:1) to afford 2-chloro-N-({4-[1-isopropyl-4-(trifluoromethyl)imidazol-2-yl]-3-methoxyphenyl}methyl)-5-nitropyrimidin-4-amine (70 mg, 15.53%) as a yellow oil.

LC-MS (ESI) m/z 471.10[M+H]

Step 5:

To a stirred solution of 2-chloro-N-({4-[1-isopropyl-4-(trifluoromethyl)imidazo]-2-yl]-3-methoxyphenyl}methyl)-5-nitropyrimidin-4-amine (150 mg, 0.319 mmol, 1 equiv) in EtOH (3 mL) and H₂O (1 mL) were added iron (88.96 mg, 1.595 mmol, 5 equiv) and NH₄Cl (136.33 mg, 2.552 mmol, 8 equiv) at room temperature under nitrogen atmosphere. The resulting mixture was stirred for 1 h at under nitrogen atmosphere. The mixture was allowed to cool down to room temperature. The resulting mixture was filtered, the filter cake was washed with MeOH (3×50 mL). The filtrate was concentrated under reduced pressure to afford 2-chloro-N4-({4-[1-isopropyl-4-(trifluoromethyl)imidazo]-2-yl]-3-methoxyphenyl}methyl)pyrimidine-4,5-diamine (200 mg, crude) as a yellow green solid. The crude product was used in the next step directly without further purification.

LC-MS (ESI) m/z 441.1[M+H]

### Step 6: 2-Chloro-8-({4-[1-Isopropyl-4-(Trifluoromethyl)Imidazol-2-Yl]-3-Methoxyphenyl}Methyl)Pteridin-7-One

To a stirred mixture of 2-chloro-N4-({4-[1-isopropyl-4-(trifluoromethyl)imidazo]-2-yl]-3-methoxyphenyl}methyl)pyrimidine-4,5-diamine (50 mg, 0.113 mmol, 1 equiv) in MeOH (1.5 mL) were added ethyl glyoxylate (92.63 mg, 0.452 mmol, 4 equiv, 50%) and AcOH (0.1 mL) at room temperature under nitrogen atmosphere. The resulting mixture was stirred for 3h at 100° C. under nitrogen atmosphere. The mixture was allowed to cool down to room temperature. The resulting mixture was diluted with water (10 mL). The resulting mixture was extracted with EtOAc (3×20 mL). The combined organic layers were washed with brine (2×20 mL), dried over anhydrous Na₂SO₄, filtered and concentrated under reduced pressure. The residue was purified by silica gel column chromatography, eluted with PE/EA (1:1) to afford 2-chloro-8-({4-[1-isopropyl-4-(trifluoromethyl)imidazol-2-yl]-3-methoxyphenyl}methyl)pteridin-7-one (50 mg, 92.06%) as a yellow oil.

LC-MS (ESI) m/z 479.10[M+H]

### Step 7: 2-(4-Cyclopropyl-6-Methoxypyrimidin-5-Yl)-8-({4-[1-Isopropyl-4-(Trifluoromethyl)Imidazol-2-Yl]-3-Methoxyphenyl}Methyl)Pteridin-7-One

To a stirred solution of 2-chloro-8-({4-[1-isopropyl-4-(trifluoromethyl)imidazo]-2-yl]-3-methoxyphenyl}methyl)pteridin-7-one (700 mg, 1.462 mmol, 1 equiv) and 4-cyclopropyl-6-methoxypyrimidin-5-ylboronic acid (425.39 mg, 2.193 mmol, 1.5 equiv) in H₂O (2 mL) and 1,4-dioxane (10 mL) were added Pd(dppf)C12 (106.96 mg, 0.146 mmol, 0.1 equiv) and K₃PO₄ (930.87 mg, 4.386 mmol, 3 equiv) at room temperature under nitrogen atmosphere. The resulting mixture was stirred for 2 h at 90° C. under nitrogen atmosphere. The mixture was allowed to cool down to room temperature. The resulting mixture was extracted with EtOAc (3×100 mL). The combined organic layers were washed with brine (2×100 mL), dried over anhydrous Na₂SO₄, filtered and concentrated under reduced pressure. The crude product (150 mg) was purified by Prep-HPLC with the following conditions (Column: Xselect CSH C18 OBD Column 30*150 mm 51 μm, n; Mobile Phase A: Water(0.1% FA), Mobile Phase B: ACN; Flow rate: 60 mL/min mL/min; Gradient: 45% B to 62% B in 9 min; Wave Length: 254 nm/220 nm nm; RT 1(min): 7.4) to afford 2-(4-cyclopropyl-6-methoxypyrimidin-5-yl)-8-({4-[1-isopropyl-4-(trifluoromethyl)imidazol-2-yl]-3-methoxyphenyl}methyl)pteridin-7-one (91.8 mg, 10.34%, purity:97.6%) as an off-white solid.

LC-MS (ESI) m/z 593.15[M+H]

¹H NMR (400 MHz, DMSO-d6) δ 9.38 (s, 1H), 8.70 (s, 1H), 8.50 (s, 1H), 8.11 (t, J=1.4 Hz, 1H), 7.26-7.16 (m, 2H), 6.93 (dd, J=7.8, 1.5 Hz, 1H), 5.49 (s, 2H), 3.96 (h, J=6.6 Hz, 1H), 3.82 (s, 3H), 3.69 (s, 3H), 1.75 (tt, J=8.2, 4.6 Hz, 1H), 1.29 (d, J=6.6 Hz, 6H), 1.02 (dq, J=6.3, 3.5 Hz, 2H), 0.76 (dd, J=7.9, 3.3 Hz, 2H).

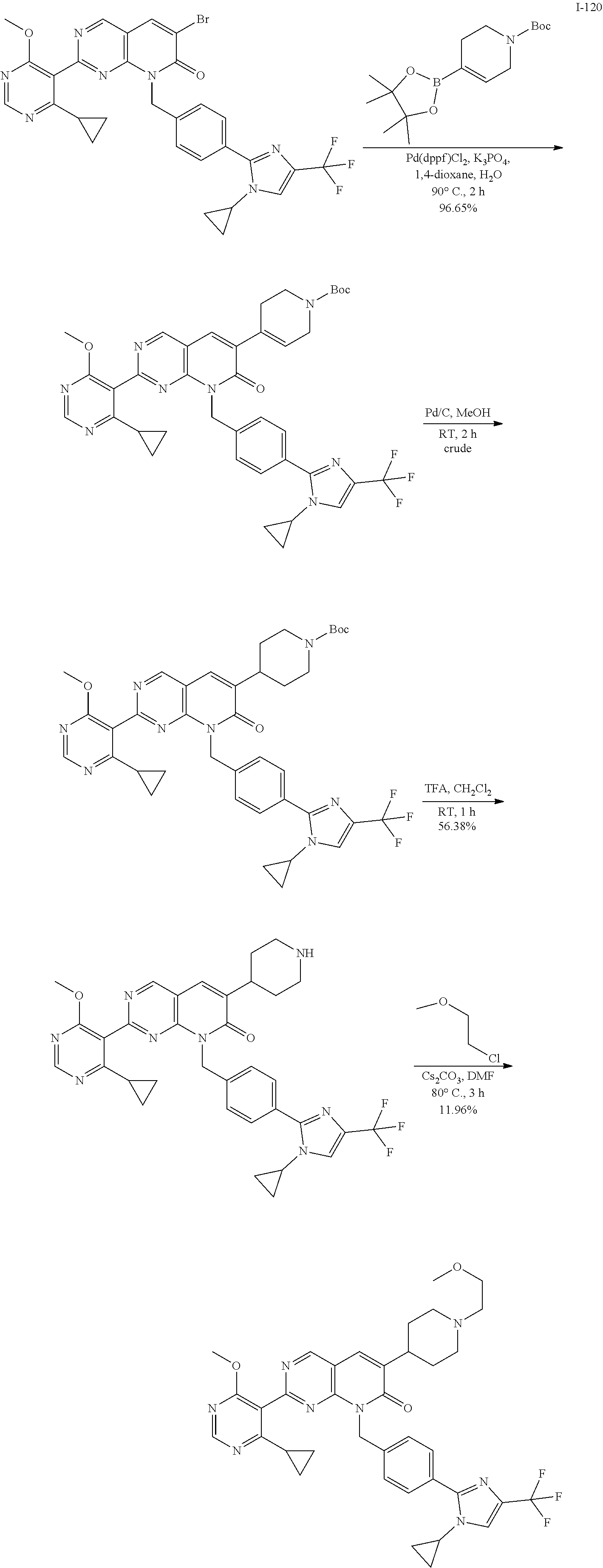

### Step 1: Tert-Butyl 4-[8-({4-[1-Cyclopropyl-4-(Trifluoromethyl)Imidazol-2-Yl]Phenyl}Methyl)-2-(4-Cyclopropyl-6-Methoxypyrimidin-5-Yl)-7-Oxopyrido[2,3-d]Pyrimidin-6-Yl]-3,6-Dihydro-211-Pyridine-1-Carboxylate

To a stirred mixture of 6-bromo-8-({4-[1-cyclopropyl-4-(trifluoromethyl)imidazol-2-yl]phenyl}methyl)-2-(4-cyclopropyl-6-methoxypyrimidin-5-yl)pyrido[2,3-d]pyrimidin-7-one (1.4 g, 2.193 mmol, 1 equiv) and tert-butyl 4-(4,4,5,5-tetramethyl-1,3,2-dioxaborolan-2-yl)-3,6-dihydro-2H-pyri di ne-1-carboxylate (1.02 g, 3.290 mmol, 1.5 equiv) in 1,4-dioxane (25 mL) and H₂O (5 mL) were added Pd(dppf)C12 (0.32 g, 0.439 mmol, 0.2 equiv) and K₃PO₄ (1.16 g, 5.482 mmol, 2.5 equiv) at room temperature under nitrogen atmosphere. The resulting mixture was stirred for 2 h at 90° C. under nitrogen atmosphere. The mixture was allowed to cool down to room temperature. The resulting mixture was diluted with water (100 mL). The resulting mixture was extracted with EtOAc (3×100 mL). The combined organic layers were washed with brine (2×100 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford tert-butyl 4-[8-({4-[1-cyclopropyl-4-(trifluoromethyl)imidazol-2-yl]phenyl}methyl)-2-(4-cyclopropyl-6-methoxypyrimidin-5-yl)-7-oxopyrido[2,3-d]pyrimidin-6-yl]-3,6-dihydro-2H-pyridine-1-carboxylate (1.57 g, 96.65%) as a brown oil.

LC-MS (ESI) m/z 741.30 [M+H]

Step 2:

To a stirred mixture of tert-butyl 4-[8-({4-[1-cyclopropyl-4-(trifluoromethyl)imidazol-2-yl]phenyl methyl)-2-(4-cyclopropyl-6-methoxypyrimidin-5-yl)-7-oxopyrido[2,3-d]pyrimidin-6-yl]-3,6-dihydro-2H-pyridine-1-carboxylate (1.57 g, 2.119 mmol, 1 equiv) in MeOH (30 mL) was added 10% Pd/C (785 mg) in portions at room temperature under hydrogen atmosphere. The resulting mixture was stirred for 2 h at room temperature under hydrogen atmosphere. The resulting mixture was filtered, the Celite pad was washed with MeOH (3×300 mL). The filtrate was concentrated under reduced pressure. This resulted in tert-butyl 4-[8-({4-[1-cyclopropyl-4-(trifluoromethyl)imidazo]-2-yl]phenyl}methyl)-2-(4-cyclopropyl-6-methoxypyrimidin-5-yl)-7-oxopyrido[2,3-d]pyrimidin-6-yl]piperidine-1-carboxylate (1.64 g, crude) as a yellow solid. The crude product was used in the next step directly without further purification.

LC-MS (ESI) m/z 743.30 [M+H]

### Step 3 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(Piperidin-4-Yl)Pyrido[2,3-d]Pyrimidin-7-One

A solution of tert-butyl 4-[8-({4-[1-cyclopropyl-4-(trifluoromethyl)imidazol-2-yl]phenyl methyl)-2-(4-cyclopropyl-6-methoxypyrimidin-5-yl)-7-oxopyrido[2,3-d]pyrimidin-6-yl]piperidine-1-carboxylate (1.64 g, 2.208 mmol, 1 equiv) in TFA (8 mL) and CH₂ Cl₂ (24 mL) was stirred for 1 h at room temperature under nitrogen atmosphere. The resulting mixture was diluted with water (100 mL). The resulting mixture was extracted with CH₂Cl₂ (3×200 mL). The combined organic layers were washed with brine (2×200 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 8-({4-[1-cyclopropyl-4-(trifluoromethyl)imidazo]-2-yl]phenyl methyl)-2-(4-cyclopropyl-6-methoxypyrimidin-5-yl)-6-(piperidin-4-yl)pyri do[2,3-d]pyrimidin-7-one (800 mg, 56.38%) as a yellow solid.

LC-MS (ESI) m/z 643.30 [M+H]

### Step 4: 8-({4-[1-cyclopropyl-4-(trifluoromethyl)imidazol-2-yl]phenyl}methyl)-2-(4-cyclopropyl-6-methoxypyrimidin-5-yl)-6-[1-(2-methoxyethyl)piperidin-4-yl]pyrido[2,3-d]pyrimidin-7-one

To a stirred solution of 8-({4-[1-cyclopropyl-4-(trifluoromethyl)imidazol-2-yl]phenyl}methyl)-2-(4-cyclopropyl-6-methoxypyrimidin-5-yl)-6-(piperidin-4-yl)pyrido[2,3-d]pyrimidin-7-one (50 mg, 0.078 mmol, 1 equiv) and ethane, 1-chloro-2-methoxy-(29.4 mg, mmol, 4 equiv) in DMF (1 mL) was added Cs₂CO₃ (76.0 mg, 0.234 mmol, 3 equiv) at room temperature under nitrogen atmosphere. The resulting mixture was stirred for 3h at under nitrogen atmosphere. The mixture was allowed to cool down to room temperature. The resulting mixture was filtered, the filter cake was washed with DMF (3×0.5 mL). The resulting mixture was purified by Prep-HPLC with the following conditions (Column: XBridge Prep Phenyl OBD Column 19*250 mm, 5 μm; Mobile Phase A: Water(10 mmol/L NH₄HCO₃), Mobile Phase B: ACN; Flow rate: 60 mL/min; Gradient: 40% B to 56 B in 8 min; Wave Length: 254 nm/220 nm; RT1(min): 8.07) to afford 8-({4-[1-cyclopropyl-4-(trifluoromethyl)imidazo]-2-yl]phenyl}methyl)-2-(4-cyclopropyl-6-methoxypyrimidin-5-yl)-6-[1-(2-methoxyethyl)piperidin-4-yl]pyrido[2,3-d]pyrimidin-7-one (6.9 mg, 11.96%) as an off-white solid.

LC-MS (ESI) m/z 701.3 [M+H]

¹H NMR: (400 MHz, DMSO-d6) δ 9.24 (s, 1H), 8.69 (s, 1H), 8.00 (s, 1H), 7.91 (d, J=1.4 Hz, 1H), 7.84-7.77 (m, 2H), 7.40 (d, J=8.3 Hz, 2H), 5.59 (s, 2H), 3.83 (s, 3H), 3.69 (tt, J=7.2, 3.9 Hz, 1H), 3.45 (t, J=5.9 Hz, 2H), 3.25 (s, 3H), 3.04-2.97 (m, 2H), 2.80 (td, J=10.4, 8.8, 6.2 Hz, 1H), 2.08 (td, J=11.8, 2.3 Hz, 2H), 1.82 (d, J=12.6 Hz, 2H), 1.73 (tt, J=8.3, 4.7 Hz, 1H), 1.60 (qd, J=12.3, 3.7 Hz, 2H), 1.05-0.96 (m, 2H), 1.00-0.90 (m, 2H), 0.93-0.83 (m, 2H), 0.77 (dq, J=10.0, 3.5 Hz, 2H).

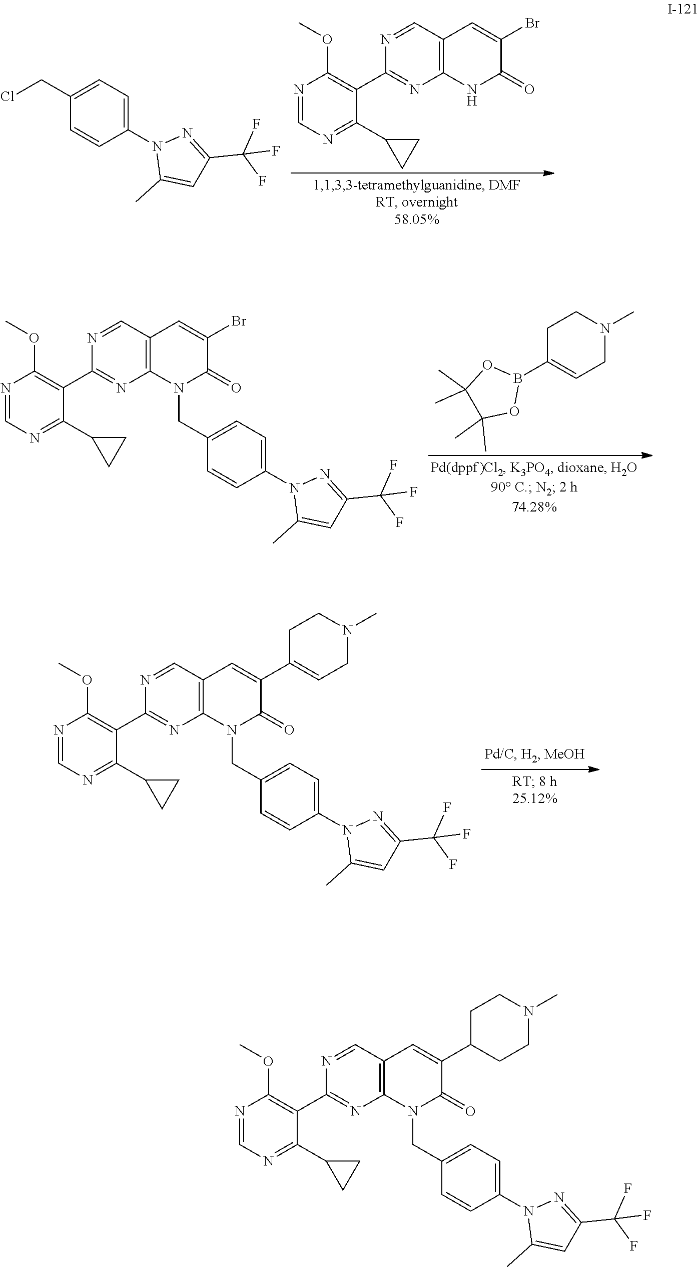

### Step 1: 6-Bromo-2-(4-Cyclopropyl-6-Methoxypyrimidin-5-Yl)-8-({4-[5-Methyl-3-(Trifluoromethyl)Pyrazol-1-Yl]Phenyl}Methyl)Pyrido[2,3-d]Pyrimidin-7-One

To a stirred solution of 1-[4-(chloromethyl)phenyl]-5-methyl-3-(trifluoromethyl)pyrazole (132.2 mg, 0.481 mmol, 0.9 equiv) and 6-bromo-2-(4-cyclopropyl-6-methoxypyrimidin-5-yl)-8H-pyrido[2,3-d]pyrimidin-7-one (200 mg, 0.534 mmol, 1.00 equiv) in DMF (3 mL) was added N,N,N′,N′-tetramethylguanidine (92.4 mg, 0.801 mmol, 1.5 equiv) at room temperature under nitrogen atmosphere. The resulting mixture was stirred for overnight at room temperature under nitrogen atmosphere. The mixture was purified by reversed-phase flash chromatography (C18 silica gel column, eluting with 0% to 100% MeCN/water (0.1% FA) in 20 min with UV detector (254 nm)) to afford 6-bromo-2-(4-cyclopropyl-6-methoxypyrimidin-5-yl)-8-({4-[5-methyl-3-(trifluoromethyl)pyrazol-1-yl]phenyl}methyl)pyri do[2,3-d]pyrimidin-7-one (190 mg, 58.05%) as a light yellow solid.

LC-MS (ESI) m/z 612.09 [M+H]

### Step 2: 2-(4-Cyclopropyl-6-Methoxypyrimidin-5-Yl)-6-(1-Methyl-3,6-Dihydro-211-Pyridin-4-Yl)-8-({4-[5-Methyl-3-(Trifluoromethyl)Pyrazol-1-Yl]Phenyl}Methyl)Pyrido[2,3-d]Pyrimidin-7-One

To a stirred solution of 6-bromo-2-(4-cyclopropyl-6-methoxypyrimidin-5-yl)-8-({4-[5-methyl-3-(trifluoromethyl)pyrazol-1-yl]phenyl}methyl)pyri do[2,3-d]pyrimidin-7-one (160 mg, 0.261 mmol, 1 equiv) and 1-methyl-4-(4,4,5,5-tetramethyl-1,3,2-dioxaborolan-2-yl)-3,6-dihydro-2H-pyridine (69.95 mg, 0.313 mmol, 1.2 equiv) in dioxane (2 mL) and H₂O (0.4 mL) were added K₃PO₄ (138.6 mg, 0.653 mmol, 2.5 equiv) and Pd(dppf)C12 (38.2 mg, 0.052 mmol, 0.2 equiv) at room temperature under nitrogen atmosphere. The resulting mixture was stirred for 2 h at 90° C. under nitrogen atmosphere. The resulting mixture was filtered, the filter cake was washed with THF (3×3 mL). The filtrate was concentrated under reduced pressure. The residue was purified by reversed-phase flash chromatography (C18 silica gel column, eluting with 0% to 100% MeCN/water (0.1% FA) in 20 min with UV detector (254 nm)) to afford 2-(4-cyclopropyl-6-methoxypyrimidin-5-yl)-6-(1-methyl-3,6-dihydro-2H-pyridin-4-yl)-8-({4-[5-methyl-3-(trifluoromethyl)pyrazol-1-yl]phenyl}methyl)pyrido[2,3-d]pyrimidin-7-one (122 mg, 74.28%) as a brown solid.

LC-MS (ESI) m/z 629.25 [M+H]

### Step 3: 2-(4-Cyclopropyl-6-Methoxypyrimidin-5-Yl)-8-({4-[5-Methyl-3-(Trifluoromethyl)Pyrazol-1-Yl]Phenyl}Methyl)-6-(1-Methylpiperidin-4-Yl)Pyrido[2,3-d]Pyrimidin-7-One

To a solution of 2-(4-cyclopropyl-6-methoxypyrimidin-5-yl)-6-(1-methyl-3,6-dihydro-2H-pyri din-4-yl)-8-({4-[5-methyl-3-(trifluoromethyl)pyrazol-1-yl]phenyl methyl)pyrido[2,3-d]pyrimidin-7-one (50 mg, 0.080 mmol, 1 equiv) in 3 mL MeOH was added 10% Pd/C (30 mg) under nitrogen atmosphere in a 50 mL round-bottom flask. The mixture was hydrogenated at room temperature for 8 h under hydrogen atmosphere using a hydrogen balloon, filtered through a Celite pad and concentrated under reduced pressure. The residue was purified by Prep-HPLC with the following conditions (Column: XBridge Prep Phenyl OBD Column 19*250 mm, 51 μm; Mobile Phase A: Water(10 mmol/L NH₄HCO₃), Mobile Phase B: ACN; Flow rate: 60 mL/min; Gradient: 39% B to 55% B in 10 min; Wave Length: 254 nm/220 nm; RT1(min): 8.58) to afford 2-(4-cyclopropyl-6-methoxypyrimidin-5-yl)-8-({4-[5-methyl-3-(trifluoromethyl)pyrazol-1-yl]phenyl methyl)-6-(1-methylpiperidin-4-yl)pyrido[2,3-d]pyrimidin-7-one (12.6 mg, 25.12%) as an off-white solid.

LC-MS (ESI) m/z 631.20 [M+H]

¹H NMR: (400 MHz, DMSO-d6) δ 9.26 (s, 1H), 8.69 (s, 1H), 7.99 (s, 1H), 7.52-7.42 (m, 4H), 6.73 (s, 1H), 5.61 (s, 2H), 3.83 (s, 3H), 2.90 (d, J=11.2 Hz, 2H), 2.78 (t, J=12.2 Hz, 1H), 2.30 (s, 3H), 2.21 (s, 3H), 2.03-1.92 (m, 2H), 1.83 (d, J=12.4 Hz, 2H), 1.72 (tt, J=8.1, 4.6 Hz, 1H), 1.67-1.55 (m, 2H), 1.02 (dq, J=6.2, 3.6 Hz, 2H), 0.78 (dq, J=10.2, 3.5 Hz, 2H).

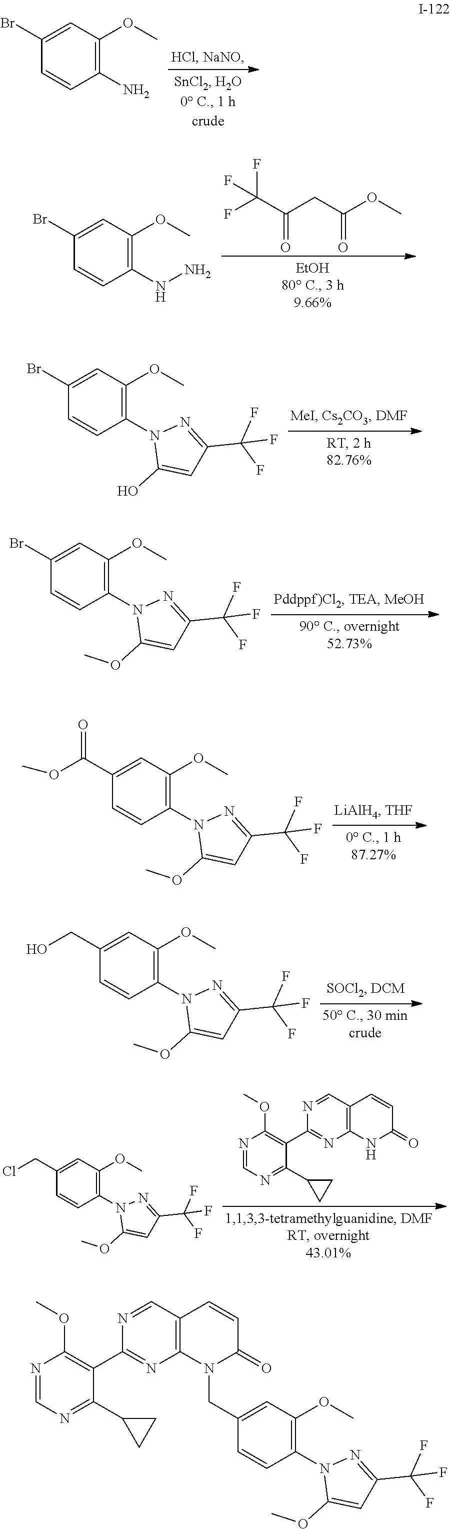

### Step 1: (4-Bromo-2-Methoxyphenyl)Hydrazine

A solution of 4-bromo-2-methoxyaniline (5 g, 24.746 mmol, 1 equiv) in 6M HCl (50 mL) was dropwise at NaNO₂ (1.71 g, 24.746 mmol, 1.0 equiv) in H₂O (2.5 ml) for 1 h at −20° C. under nitrogen atmosphere followed by the addition of SnCl₂ (16.60 g, 86.611 mmol, 3.5 equiv) in 6M HCl(50 ml) dropwise at −20° C. The resulting mixture was stirred for 1 h at under nitrogen atmosphere. The precipitated solids were collected by filtration and washed with water (3×100 mL). The resulting solid was dried under vacuum. This resulted in (4-bromo-2-methoxyphenyl)hydrazine (9.2 g, crude) as a brown solid. The crude product was used in the next step directly without further purification.

LC-MS (ESI) m/z 217.1 [M+H]

### Step 2 2-(4-Bromo-2-Methoxyphenyl)-5-(Trifluoromethyl)Pyrazol-3-0l

To a stirred solution of (4-bromo-2-methoxyphenyl)hydrazine (2 g, 9.214 mmol, 1 equiv) in EtOH (100 mL) was added ethyl 4,4,4-trifluoro-3-oxobutanoate (2.54 g, 13.821 mmol, 1.50 equiv) at room temperature under nitrogen atmosphere. The resulting mixture was stirred for 3h at 80° C. under nitrogen atmosphere. The mixture was allowed to cool down to room temperature. The resulting mixture was concentrated under reduced pressure. The residue was purified by reversed-phase flash chromatography with the following conditions: column, C18 silica gel; mobile phase, MeCN in Water (0.1% NH 3.H₂O), 0% to 100% gradient in 10 min; detector, UV 254 nm. This resulted in 2-(4-bromo-2-methoxyphenyl)-5-(trifluoromethyl)pyrazol-3-01 (300 mg, 9.66%) as a light yellow solid.

LC-MS (ESI) m/z 337.1 [M+H]

### Step 3: 1-(4-Bromo-2-Methoxyphenyl)-5-Methoxy-3-(Trifluoromethyl)Pyrazole

To a stirred solution of 2-(4-bromo-2-methoxyphenyl)-5-(trifluoromethyl)pyrazol-3-01 (1.16 g, 3.441 mmol, 1 equiv) and Cs₂CO₃ (2.24 g, 6.882 mmol, 2 equiv) in DMF (4 mL) was added MeI (0.59 g, 4.129 mmol, 1.2 equiv) dropwise at 0° C. under nitrogen atmosphere. The resulting mixture was stirred for 2 h at room temperature under nitrogen atmosphere. The reaction was quenched by the addition of Water (100 mL) at 0° C. The resulting mixture was extracted with EtOAc (3×100 mL). The combined organic layers were washed with brine (2×50 mL), dried over anhydrous Na₂SO₄, filtered and concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm. This resulted in 1-(4-bromo-2-m ethoxyphenyl)-5-m ethoxy-3-(trifluoromethyl)pyrazol e (1 g, 82.76%) as a light yellow oil.

LC-MS (ESI) m/z 351.2 [M+H]

### Step 4 Methyl 3-Methoxy-4-[5-Methoxy-3-(Trifluoromethyl)Pyrazol-1-Yl]Benzoate

To a stirred 1-(4-bromo-2-methoxyphenyl)-5-methoxy-3-(trifluoromethyl)pyrazole (500 mg, 1.424 mmol, 1 equiv) in MeOH (10 mL) were added Pd(dppf)Cl₂ (208.3 mg, 0.285 mmol, 0.2 equiv) and TEA (432.3 mg, 4.272 mmol, 3 equiv) at room temperature under carbon monoxide atmosphere. The resulting mixture was stirred for overnight at 90° C. under carbon monoxide atmosphere. The mixture was allowed to cool down to room temperature. The resulting mixture was filtered, the filter cake was washed with MeOH (3×100 mL). The filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm. This resulted in methyl 3-m ethoxy-4-[5-m ethoxy-3-(trifluoromethyl)pyrazol-1-yl]benzoate (248 mg, 52.73%) as a yellow oil.

LC-MS (ESI) m/z 331.15[M+H]

### Step 5: {3-Methoxy-4-[5-Methoxy-3-(Trifluoromethyl)Pyrazol-1-Yl]Phenyl}Methanol

To a stirred solution of methyl 3-m ethoxy-4-[5-m ethoxy-3-(trifluoromethyl)pyrazol-1-yl]benzoate (313 mg, 0.948 mmol, 1 equiv) in THF (6 mL) was added LiAlH₄ (107.9 mg, 2.843 mmol, 3.00 equiv) in portions at 0° C. under nitrogen atmosphere. The resulting mixture was stirred for 1 h at 0° C. under nitrogen atmosphere. The reaction was quenched with 10H₂O.Na₂SO₄(aq) at 0° C. The resulting mixture was filtered, the filter cake was washed with THF (3×50 mL). The filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm. This resulted in {3-m ethoxy-4-[5-m ethoxy-3-(trifluoromethyl)pyrazol-1-yl]phenyl}methanol (250 mg, 87.27%) as a light yellow oil.

LC-MS (ESI) m/z 303.1[M+H]

### Step 6: 1-[4-(Chloromethyl)-2-Methoxyphenyl]-5-Methoxy-3-(Trifluoromethyl)Pyrazole

To a stirred solution of {3-methoxy-4-[5-methoxy-3-(trifluoromethyl)pyrazol-1-yl]phenyl}methanol (250 mg, 0.827 mmol, 1 equiv) in DCM (5 mL) was added SOCl₂ (295.1 mg, 2.481 mmol, 3.00 equiv) at room temperature under nitrogen atmosphere. The resulting mixture was stirred for 30 min at 50° C. under nitrogen atmosphere. The mixture was allowed to cool down to room temperature. The reaction was quenched by the addition of Water (20 mL) at 0° C. The resulting mixture was extracted with DCM(3×50 mL). The combined organic layers were washed with brine (2×50 mL), dried over anhydrous Na₂SO₄. After filtration, the filtrate was concentrated under reduced pressure. This resulted in 1-[4-(chloromethyl)-2-methoxyphenyl]-5-methoxy-3-(trifluoromethyl)pyrazole (280 mg, crude) as a brown oil. The crude product was used in the next step directly without further purification.

LC-MS (ESI) m/z 321.1[M+H]

### Step 7: 2-(4-Cyclopropyl-6-Methoxypyrimidin-5-Yl)-8-({3-Methoxy-4-[5-Methoxy-3-(Trifluoromethyl)Pyrazol-1-Yl]Phenyl}Methyl)Pyrido[2,3-d]Pyrimidin-7-One

To a stirred solution of 1-[4-(chloromethyl)-2-methoxyphenyl]-5-methoxy-3-(trifluoromethyl)pyrazole (150 mg, 0.468 mmol, 1 equiv) and 2-(4-cyclopropyl-6-methoxypyrimidin-5-yl)-8H-pyrido[2,3-d]pyrimidin-7-one (138.1 mg, 0.468 mmol, 1 equiv) in DMF (3 mL) was added 1,1,3,3-tetramethylguanidine (80.8 mg, 0.702 mmol, 1.5 equiv) at room temperature under nitrogen atmosphere. The resulting mixture was stirred for overnight at room temperature under nitrogen atmosphere. The resulting mixture was filtered, the filter cake was washed with DMF (2×0.5 mL). The resulting mixture was purified by Prep-HPLC with the following conditions (Column: YMC-Actus Triart C18 ExRS 30*150 mm, 5 μm; Mobile Phase A: Water(0.05%NH₃H₂O), Mobile Phase B: ACN; Flow rate: 60 mL/min; Gradient: 42% B to 59% B in 10 min; Wave Length: 254 nm/220 nm; RT1(min): 11.15) to afford 2-(4-cyclopropyl-6-methoxypyrimidin-5-yl)-8-({3-methoxy-4-[5-methoxy-3-(trifluoromethyl)pyrazol-1-yl]phenyl}methyl)pyri do[2,3-d]pyrimidin-7-one (117.4 mg, 43.01%) as an off-white solid.

LC-MS (ESI) m/z 580.25[M+H]

¹H NMR: (400 MHz, DMSO-d6) δ 9.29 (s, 1H), 8.69 (s, 1H), 8.17 (d, J=9.5 Hz, 1H), 7.26-7.19 (m, 2H), 6.92 (d, J=9.5 Hz, 1H), 6.79 (dd, J=8.1, 1.7 Hz, 1H), 6.32 (s, 1H), 5.56 (s, 2H), 3.85 (s, 3H), 3.80 (s, 3H), 3.65 (s, 3H), 1.71 (tt, J=8.2, 4.6 Hz, 1H), 1.01 (dq, J=5.9, 3.6 Hz, 2H), 0.78 (dt, J=8.2, 3.4 Hz, 2H).

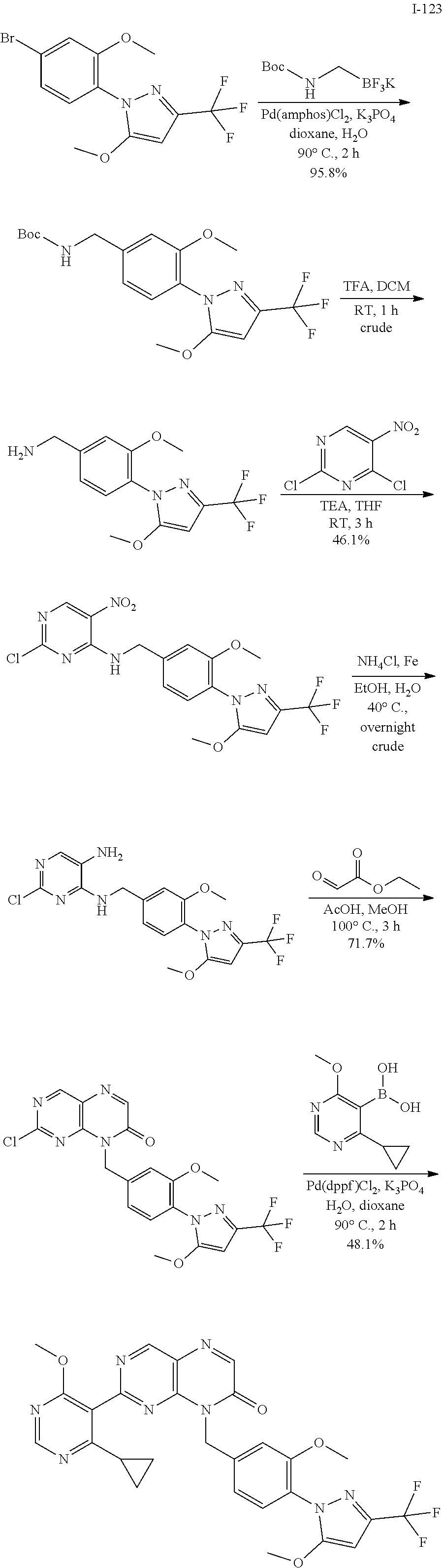

### Step 1 Tert-Butyl N-({3-Methoxy-4-[5-Methoxy-3-(Trifluoromethyl)Pyrazol-1-Yl]Phenyl}Methyl)Carbamate

To a stirred mixture of 1-(4-bromo-2-methoxyphenyl)-5-methoxy-3-(trifluoromethyl)pyrazole (420 mg, 1.196 mmol, 1 equiv) and tert-butyl N-[(trifluoro-lambda4-boranyl)methyl]carbamate potassium (425.4 mg, 1.794 mmol, 1.5 equiv) in H₂O (1.5 mL) and dioxane (7.5 mL) were added K₃PO₄ (507.8 mg, 2.392 mmol, 2 equiv) and Pd(amphos)C12 (169.4 mg, 0.239 mmol, 0.2 equiv) at room temperature under nitrogen atmosphere. The resulting mixture was stirred for 2 h at under nitrogen atmosphere. The mixture was allowed to cool down to room temperature. The resulting mixture was diluted with water (100 mL). The resulting mixture was extracted with EtOAc (3×100 mL). The combined organic layers were washed with brine (3×50 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford tert-butyl N-({3-methoxy-4-[5-methoxy-3-(trifluoromethyl)pyrazol-1-yl]phenyl}methyl)carbamate (460 mg, as a brown oil.

LC-MS (ESI) m/z 402.2 [M+H]

### Step 2: 1-{3-Methoxy-4-[5-Methoxy-3-(Trifluoromethyl)Pyrazol-1-Yl]Phenyl}Methanamine

A mixture of tert-butyl N-({3-m ethoxy-4-[5 -m ethoxy-3 -(trifluoromethyl)pyrazol-1-yl]phenyl methyl)carbamate (450 mg, 1.121 mmol, 1 equiv) in TFA (2 mL) and DCM (6 mL) was stirred for 1 h at room temperature under nitrogen atmosphere. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford 1-{3-methoxy-4-[5-methoxy-3-(trifluoromethyl)pyrazol-1-yl]phenyl}methanamine (490 mg, crude) as a yellow oil.

LC-MS (ESI) m/z 302.1 [M+H]

### Step 3: 2-Chloro-N-({3-Methoxy-4-[5-Methoxy-3-(Trifluoromethyl)Pyrazol-1-Yl]Phenyl}Methyl)-5-Nitropyrimidin-4-Amine

To a stirred mixture of 1-{3-methoxy-4-[5-methoxy-3-(trifluoromethyl)pyrazol-1-yl]phenyl methanamine (470 mg, 1.560 mmol, 1 equiv) and 2,4-dichloro-5-nitropyrimidine (605.2 mg, 3.120 mmol, 2 equiv) in THF (10 mL) was added TEA (473.6 mg, 4.680 mmol, 3 equiv) at 0° C. under nitrogen atmosphere. The resulting mixture was stirred for 3 h at room temperature under nitrogen atmosphere. The resulting mixture was diluted with water (50 mL). The resulting mixture was extracted with EtOAc (3×50 mL). The combined organic layers were washed with brine (3×50 mL), dried over anhydrous Na₂SO₄, filtered and concentrated under reduced pressure. The residue was purified by silica gel column chromatography, (eluting with PE/EA (5:1)) to afford 2-chloro-N-({3-methoxy-4-[5-m ethoxy-3-(trifluoromethyl)pyrazol-1-yl]phenyl methyl)-5-nitropyrimidin-4-amine (330 mg, 46.1%) as a yellow solid.

LC-MS (ESI) m/z 459.1 [M+H]

### Step 4: 2-Chloro-N4-({3-Methoxy-4-[5-Methoxy-3-(Trifluoromethyl)Pyrazol-1-Yl]Phenyl}Methyl)Pyrimidine-4,5-Diamine

A mixture of 2-chloro-N-({3-m ethoxy-4-[5-m ethoxy-3-(trifluoromethyl)pyrazol-1-yl]phenyl methyl)-5-nitropyrimidin-4-amine (310 mg, 0.676 mmol, 1 equiv), NH 4 C1 (180.7 mg, 3.380 mmol, 5 equiv) and Fe (377.3 mg, 6.760 mmol, 10 equiv) in EtOH (4.8 mL) and H₂O (1.2 mL) was stirred for overnight at 40° C. under nitrogen atmosphere. The mixture was allowed to cool down to room temperature. The resulting mixture was filtered, the filter cake was washed with MeOH (3×50 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford 2-chloro-N4-({3-methoxy-4-[5-methoxy-3-(trifluoromethyl)pyrazol-1-yl]phenyl}methyl)pyrimidine-4,5-di amine (300 mg, crude) as a yellow solid.

LC-MS (ESI) m/z 429.1 [M+H]

### Step 5: 2-Chloro-8-({3-Methoxy-4-[5-Methoxy-3-(Trifluoromethyl)Pyrazol-1-Yl]Phenyl}Methyl)Pteridin-7-One

A mixture of 2-chloro-N4-({3-m ethoxy-4-[5-m ethoxy-3-(trifluoromethyl)pyrazol-1-yl]phenyl}methyl)pyrimidine-4,5-diamine (320 mg, 0.746 mmol, 1 equiv),ethyl glyoxylate (228.5 mg, 2.238 mmol, 3 equiv) and AcOH (22.4 mg, 0.373 mmol, 0.5 equiv) in MeOH (6.5 mL) was stirred for 3 h at 100° C. under nitrogen atmosphere. The mixture was allowed to cool down to room temperature. The resulting mixture was diluted with water (100 mL). The resulting mixture was extracted with EtOAc (3×100 mL). The combined organic layers were washed with brine (3×50 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 2-chloro-8-({3-methoxy-4-[5-m ethoxy-3-(trifluoromethyl)pyrazol-1-yl]phenyl 1methyl)pteridin-7-one (250 mg, 71.76%) as a yellow oil.

LC-MS (ESI) m/z 467.1 [M+H]

### Step 6: 2-(4-Cyclopropyl-6-Methoxypyrimidin-5-Yl)-8-({3-Methoxy-4-[5-Methoxy-3-(Trifluoromethyl)Pyrazol-1-Yl]Phenyl}Methyl)Pteridin-7-One

To a stirred mixture of 2-chloro-8-(3-m ethoxy-4-[5-m ethoxy-3-(trifluoromethyl)pyrazol-1-yl]phenyl}methyl)pteridin-7-one (120 mg, 0.257 mmol, 1 equiv) and 4-cyclopropyl-6-methoxypyrimidin-5-ylboronic acid (74.8 mg, 0.386 mmol, 1.5 equiv) in H₂O (0.5 mL) and dioxane (2 mL) were added Pd(dppf)C12 (37.6 mg, 0.051 mmol, 0.2 equiv) and K₃PO₄ (136.4 mg, 0.643 mmol, 2.5 equiv) at room temperature under nitrogen atmosphere. The resulting mixture was stirred for 2 h at under nitrogen atmosphere. The mixture was allowed to cool down to room temperature. The resulting mixture was filtered, the filter cake was washed with THF (3×10 mL). The filtrate was concentrated under reduced pressure. The crude product (130 mg) was purified by Prep-HPLC with the following conditions (Column: Xselect CSH C18 OBD Column 30*150 mm 51 μm; Mobile Phase A: Water(0.1% FA), Mobile Phase B: ACN; Flow rate: 60 mL/min; Gradient: 43% B to 57% B in 9 min; Wave Length: 254 nm/220 nm; RT1(min): 8.48) to afford 2-(4-cyclopropyl-6-methoxypyrimidin-5-yl)-8-({3-methoxy-4-[5-m ethoxy-3-(trifluoromethyl)pyrazol-1-yl]phenyl methyl)pteridin-7-one (72.9 mg, 48.17%) as an off-white solid.

LC-MS (ESI) m/z 581.2 [M+H]

¹H NMR-(400 MHz, DMSO-d6) δ 9.37 (s, 1H), 8.70 (s, 1H), 8.49 (s, 1H), 7.28-7.21 (m, 2H), 6.94 (dd, J=8.1, 1.7 Hz, 1H), 6.33 (s, 1H), 5.47 (s, 2H), 3.83 (d, J=15.0 Hz, 6H), 3.67 (s, 3H), 1.73 (dt, J=7.9, 3.5 Hz, 1H), 1.02 (p, J=3.7 Hz, 2H), 0.79 (dq, J=6.9, 3.4 Hz, 2H).

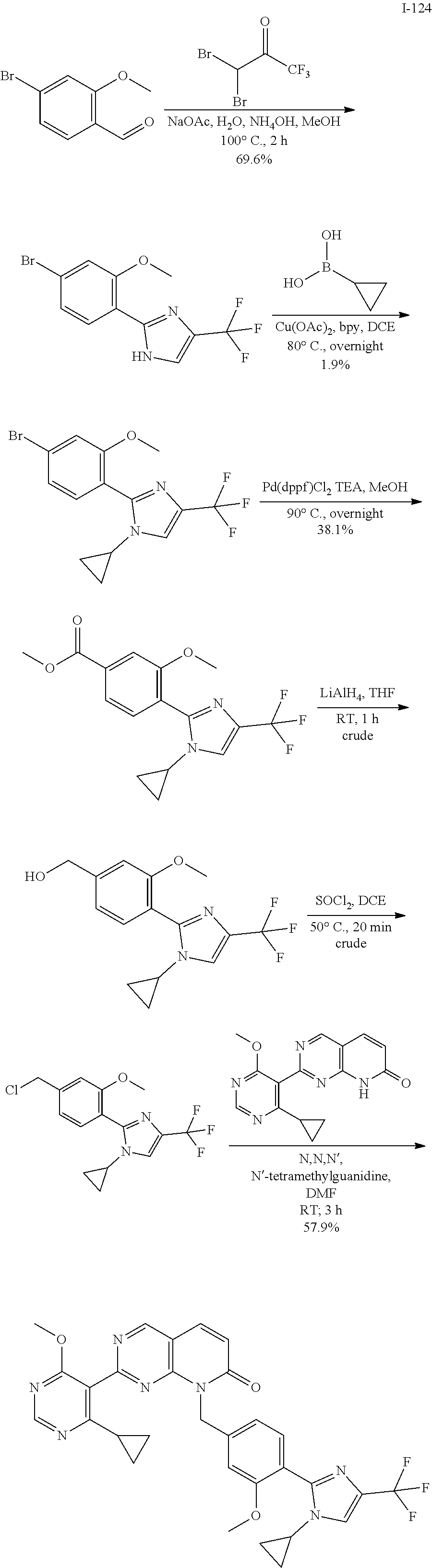

### Step 1: 2-(4-Bromo-2-Methoxyphenyl)-4-(Trifluoromethyl)-1H-Imidazole

A solution of 3,3-dibromo-1,1,1-trifluoropropan-2-one (7.1 g, 26.274 mmol, 1.13 equiv) and NaOAc (2.2 g, 26.274 mmol, 1.13 equiv) in H₂O (10 mL) was stirred for 1 h at 100° C. under nitrogen atmosphere. The mixture was allowed to cool down to room temperature. To the above mixture were added 4-bromo-2-methoxybenzaldehyde (5 g, 23.251 mmol, 1 equiv), MeOH (75 mL) and NH₄OH (18 mL) The resulting mixture was stirred for additional 40 min at room temperature. The resulting mixture was stirred for 2 h at 100° C. under nitrogen atmosphere. The mixture was allowed to cool down to room temperature. The reaction was quenched by the addition of Water (50 mL) at 0° C. The resulting mixture was extracted with EtOAc (3×100 mL). The combined organic layers were washed with brine (3×100 mL), dried over anhydrous Na₂SO₄, filtrated and concentrated under reduced pressure. The residue was purified by silica gel column chromatography, eluting with PE/EA (5:1) to afford 2-(4-bromo-2-methoxyphenyl)-4-(trifluoromethyl)-1H-imidazole (5.2 g, 69.6%) as a yellow solid.

LC-MS (ESI) m/z 322.1 [M+H]

### Step 2: 2-(4-Bromo-2-Methoxyphenyl)-1-Cyclopropyl-4-(Trifluoromethyl)Imidazole

To a stirred solution of 2-(4-bromo-2-methoxyphenyl)-4-(trifluoromethyl)-1H-imidazole (10 g, 31.143 mmol, 1 equiv) and cyclopropylboronic acid (5.4 g, 62.286 mmol, 2 equiv) in DCE (100 mL) were added bipyridyl (14.6 g, 93.429 mmol, 3 equiv) and Cu(OAc)₂ (16.9 g, 93.429 mmol, 3 equiv) at room temperature under oxygen atmosphere. The resulting mixture was stirred for overnight at 80° C. under oxygen atmosphere. The mixture was allowed to cool down to room temperature. The resulting mixture was filtered, the filter cake was washed with DCM (3×100 mL). The filtrate was concentrated under reduced pressure. The residue was purified by silica gel column chromatography, eluting with PE/EA (12:1) to afford 2-(4-bromo-2-methoxyphenyl)-1-cyclopropyl-4-(trifluoromethyl)imidazole (220 mg, 1.96%) as a yellow solid.

LC-MS (ESI) m/z 361.1 [M+H]

### Step 3: Methyl 4-[1-Cyclopropyl-4-(Trifluoromethyl)Imidazol-2-Yl]-3-Methoxybenzoate

To a stirred solution of 2-(4-bromo-2-methoxyphenyl)-1-cyclopropyl-4-(trifluoromethyl)imidazole (390 mg, 1.080 mmol, 1 equiv) and TEA (327.8 mg, 3.240 mmol, 3 equiv) in MeOH (5 mL) was added Pd(dppf)C12 158.1 mg, 0.216 mmol, 0.2 equiv) at room temperature under air atmosphere. The resulting mixture was stirred for overnight at under carbon monoxide atmosphere. The mixture was allowed to cool down to room temperature. The resulting mixture was diluted with water (50 mL). The resulting mixture was extracted with EtOAc (3×50 mL). The combined organic layers were washed with brine (3×mL), dried over anhydrous Na₂SO₄, filtrated and concentrated under reduced pressure. The residue was purified by silica gel column chromatography, eluting with PE/EA (10:1) to afford methyl 4-[1-cyclopropyl-4-(trifluoromethyl)imidazol-2-yl]-3-methoxybenzoate (140 mg, 38.1%) as a yellow solid.

LC-MS (ESI) m/z 341.3 [M+H]

### Step 4: {4-[1-Cyclopropyl-4-(Trifluoromethyl)Imidazol-2-Yl]-3-Methoxyphenyl}Methanol

To a stirred solution of methyl 4-[1-cyclopropyl-4-(trifluoromethyl)imidazo]-2-yl]-3-methoxybenzoate (100 mg, 0.294 mmol, 1 equiv) in tetrahydrofuran (2 mL) was added LiAlH₄ (33.5 mg, 0.882 mmol, 3 equiv) in portions at 0° C. under nitrogen atmosphere. The resulting mixture was stirred for 1 h at room temperature under nitrogen atmosphere. The reaction was quenched with Na₂SO₄.10H₂O at 0° C. The resulting mixture was filtered, the filter cake was washed with THF (3×10 mL). The filtrate was concentrated under reduced pressure. The residue was purified by reversed-phase flash chromatography C18 silica gel column, eluting with 10% to 50% MeCN/water (0.1% FA) in 10 min with UV detector (254 nm) to afford {4-[1-cyclopropyl-4-(trifluoromethyl)imidazo]-2-yl]-3-methoxyphenyl}methanol (93 mg, crude) as a yellow oil.

LC-MS (ESI) m/z 313.2 [M+H]

### Step 5: 2-[4-(Chloromethyl)-2-Methoxyphenyl]-1-Cyclopropyl-4-(Trifluoromethyl)Imidazole

To a stirred solution of {4-[1-cyclopropyl-4-(trifluoromethyl)imidazo]-2-yl]-3-methoxyphenyl}methanol (93 mg, 0.298 mmol, 1 equiv) in DCE (2 mL) was added thionyl chloride (106.3 mg, 0.894 mmol, 3 equiv) dropwise at room temperature under nitrogen atmosphere. The resulting mixture was stirred for 20 min at room temperature under nitrogen atmosphere. The resulting mixture was extracted with EtOAc (3×50 mL). The combined organic layers were washed with brine (3×50 mL), dried over anhydrous Na₂SO₄, filtrated and concentrated under reduced pressure to afford 2-[4-(chloromethyl)-2-m ethoxyphenyl]-1-cyclopropyl-4-(trifluoromethyl)imidazole (120 mg, crude) as a white solid.

LC-MS (ESI) m/z 331.7 [M+H]

### Step 6: 8-({4-[1-Cyclopropyl-4-(Trifluoromethyl)Imidazol-2-Yl]-3-Methoxyphenyl}Methyl)-2-(4-Cyclopropyl-6-Methoxypyrimidin-5-Yl)Pyrido[2,3-d]Pyrimidin-7-One

To a stirred solution of 2-[4-(chloromethyl)-2-methoxyphenyl]-1-cyclopropyl-4-(trifluoromethyl)imidazole (100 mg, 0.302 mmol, 1 equiv) and 2-(4-cyclopropyl-6-methoxypyrimidin-5-yl)-8H-pyrido[2,3-d]pyrimidin-7-one (178.6 mg, 0.604 mmol, 2 equiv) in DMF (1 mL) was added 1,1,3,3-tetramethylguanidine (69.7 mg, 0.604 mmol, 2 equiv) at room temperature under nitrogen atmosphere. The resulting mixture was stirred for 3h at room temperature under nitrogen atmosphere. The resulting mixture was purified by Prep-HPLC with the following conditions (Column: XBridge Prep Phenyl OBD Column 19*250 mm, 51 μm; Mobile Phase A: Water(0.1% FA), Mobile Phase B: MeOH; Flow rate: 60 mL/min; Gradient: 54% B to 70% B in 10 min; Wave Length: 254 nm/220 nm nm; RT1(min): 9.43) to afford 8-({4-[1-cyclopropyl-4-(trifluoromethyl)imidazol-2-yl]-3-methoxyphenyl methyl)-2-(4-cyclopropyl-6-methoxypyrimidin-5-yl)pyrido[2,3-d]pyrimidin-7-one (103.3 mg, 57.95%) as an off-white solid.

LC-MS (ESI) m/z 590.5 [M+H]

¹H NMR (400 MHz, DMSO-d6) δ 9.30 (s, 1H), 8.69 (s, 1H), 8.17 (d, J=9.5 Hz, 1H), 7.84 (s, 1H), 7.22 (d, J=7.8 Hz, 1H), 7.17 (s, 1H), 6.92 (d, J=9.5 Hz, 1H), 6.78 (dd, J=7.8, 1.5 Hz, 1H), 5.57 (s, 2H), 3.81 (s, 3H), 3.70 (s, 3H), 3.28 (m, 1H), 1.74 (tt, J=8.3, 4.6 Hz, 1H), 1.01 (p, J=3.7 Hz, 2H), 0.78 (dt, J=7.8, 3.4 Hz, 2H), 0.73 (d, J=5.6 Hz, 4H).

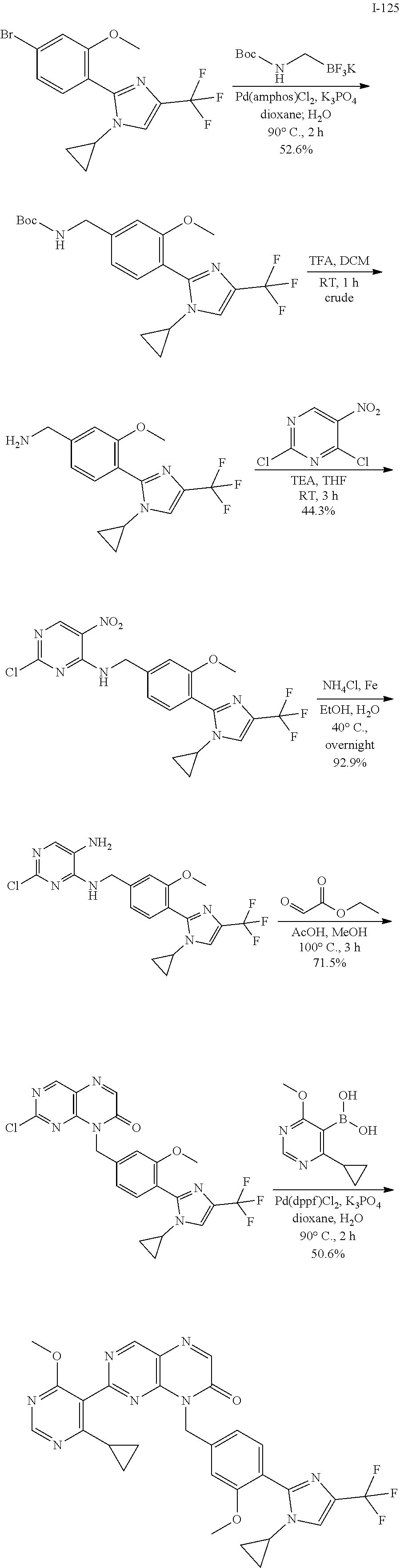

### Step 1: Tert-Butyl N-({4-[1-Cyclopropyl-4-(Trifluoromethyl)Imidazol-2-Yl]-3-Methoxyphenyl}Methyl)Carbamate

To a stirred mixture of 2-(4-bromo-2-methoxyphenyl)-1-cyclopropyl-4-(trifluoromethyl)imidazole (700 mg, 1.938 mmol, 1 equiv) and tert-butyl N-[(trifluoro-lambda4-boranyl)methyl]carbamate potassium (689.2 mg, 2.907 mmol, 1.5 equiv) in dioxane (12 mL) and H₂O (2.4 mL) were added K₃PO₄ (822.8 mg, 3.876 mmol, 2 equiv) and Pd(amphos)C12 (274.5 mg, 0.388 mmol, 0.2 equiv) at room temperature under nitrogen atmosphere. The resulting mixture was stirred for 2 h at 90° C. under nitrogen atmosphere. The mixture was allowed to cool down to room temperature. The resulting mixture was diluted with water (100 mL). The resulting mixture was extracted with EtOAc (3×100 mL). The combined organic layers were washed with brine (3×100 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford tert-butyl N-({4-[1-cyclopropyl-4-(trifluoromethyl)imidazol-2-yl]-3-methoxyphenyl}methyl)carbamate (420 mg, 52.6%) as a yellow oil.

LC-MS (ESI) m/z 412.2 [M+H]

### Step 2: 1-{4-[1-Cyclopropyl-4-(Trifluoromethyl)Imidazol-2-Yl]-3-Methoxyphenyl}Methanamine

A mixture of tert-butyl N-({4-[1-cyclopropyl-4-(trifluoromethyl)imidazo]-2-yl]-3-methoxyphenyl}methyl)carbamate (400 mg, 0.972 mmol, 1 equiv) in TFA (2 mL) and DCM (6 mL) was stirred for 1 h at room temperature under nitrogen atmosphere. The resulting mixture was concentrated under reduced pressure. The residue was purified by reverse flash chromatography (C18 silica gel column, eluting with 0% to 100% MeCN/water (0.1% FA) in 10 min with UV detector (254 nm)) to afford 1-{4-[1-cyclopropyl-4-(trifluoromethyl)imidazo]-2-yl]-3-methoxyphenyl}methanamine (390 mg, crude) as a yellow oil.

LC-MS (ESI) m/z 312.1 [M+H]

### Step 3. 2-Chloro-N-({4-[1-Cyclopropyl-4-(Trifluoromethyl)Imidazol-2-Yl]-3-Methoxyphenyl}Methyl)-5-Nitropyrimidin-4-Amine

To a stirred mixture of 1-{4-[1-cyclopropyl-4-(trifluoromethyl)imidazo]-2-yl]-3-methoxyphenyl}methanamine (374 mg, 1.201 mmol, 1 equiv) and 2,4-dichloro-5-nitropyrimidine (466.1 mg, 2.402 mmol, 2 equiv) in THF (7.5 mL) was added TEA (364.7 mg, 3.603 mmol, 3 equiv) at 0° C. under nitrogen atmosphere. The resulting mixture was stirred for 3 h at room temperature under nitrogen atmosphere. The resulting mixture was diluted with water (50 mL). The resulting mixture was extracted with EtOAc (3×50 mL). The combined organic layers were washed with brine (3×50 mL), dried over anhydrous Na₂SO₄, filtered and concentrated under reduced pressure. The residue was purified by silica gel column chromatography, (eluting with PE/EA (3:1)) to afford 2-chloro-N-({4-[1-cyclopropyl-4-(trifluoromethyl)imidazol-2-yl]-3-methoxyphenyl}methyl)-5-nitropyrimidin-4-amine (250 mg, 44.3%) as a yellow oil.

LC-MS (ESI) m/z 469.1 [M+H]

### Step 4. 2-Chloro-N4-({4-[1-Cyclopropyl-4-(Trifluoromethyl)Imidazol-2-Yl]-3-Methoxyphenyl}Methyl)Pyrimidine-4,5-Diamine

A mixture of 2-chloro-N-({4-[1-cyclopropyl-4-(trifluoromethyl)imidazol-2-yl]-3-methoxyphenyl methyl)-5-nitropyrimidin-4-amine (230 mg, 0.491 mmol, 1 equiv), NH 4 C1 (131.2 mg, 2.455 mmol, 5 equiv) and Fe (273.9 mg, 4.910 mmol, 10 equiv) in EtOH (3.6 mL) and H₂O (0.9 mL) was stirred for overnight at 40° C. under nitrogen atmosphere. The mixture was allowed to cool down to room temperature. The resulting mixture was filtered, the filter cake was washed with MeOH (3×50 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford 2-chloro-N4-({4-[1-cyclopropyl-4-(trifluoromethyl)imidazo]-2-yl]-3-methoxyphenyl}methyl)pyrimidine-4, 5-di amine (200 mg, 92.9%) as a yellow oil.

LC-MS (ESI) m/z 439.1 [M+H]

### Step 5: 2-Chloro-8-({4-[1-Cyclopropyl-4-(Trifluoromethyl)Imidazol-2-Yl]-3-Methoxyphenyl}Methyl)Pteridin-7-One

A mixture of 2-chloro-N4-({4-[1-cyclopropyl-4-(trifluoromethyl)imidazol-2-yl]-3-methoxyphenyl}methyl)pyrimidine-4,5-diamine (180 mg, 0.410 mmol, 1 equiv),AcOH (12.3 mg, 0.205 mmol, 0.5 equiv) and ethyl glyoxylate (251.2 mg, 1.230 mmol, 3 equiv, 50%) in MeOH (4 mL) was stirred for 3 h at 100° C. under nitrogen atmosphere. The mixture was allowed to cool down to room temperature. The resulting mixture was diluted with water (50 mL). The resulting mixture was extracted with EtOAc (3×50 mL). The combined organic layers were washed with brine (3×50 mL), dried over anhydrous Na₂SO₄, filtered and concentrated under reduced pressure. The residue was purified by silica gel column chromatography, (eluting with PE/EA (5:1)) to afford 2-chloro-8-({4-[1-cyclopropyl-4-(trifluoromethyl)imidazol-2-yl]-3-methoxyphenyl methyl)pteridin-7-one (140 mg, 71.5%) as an off-white solid.

LC-MS (ESI) m/z 477.1 [M+H]

### Step 6: 8-({4-[1-Cyclopropyl-4-(Trifluoromethyl)Imidazol-2-Yl]-3-Methoxyphenyl}Methyl)-2-(4-Cyclopropyl-6-Methoxypyrimidin-5-Yl)Pteridin-7-One

To a stirred mixture of 2-chloro-8-({4-[1-cyclopropyl-4-(trifluoromethyl)imidazo]-2-yl]-3-methoxyphenyl}methyl)pteridin-7-one (120 mg, 0.252 mmol, 1 equiv) and 4-cyclopropyl-6-methoxypyrimidin-5-ylboronic acid (73.2 mg, 0.378 mmol, 1.5 equiv) in H₂O (0.5 mL) and dioxane (2 mL) were added K₃PO₄ (133.5 mg, 0.630 mmol, 2.5 equiv) and Pd(dppf)C12 (36.8 mg, 0.050 mmol, 0.2 equiv) at room temperature under nitrogen atmosphere. The resulting mixture was stirred for 2 h at 90° C. under nitrogen atmosphere. The mixture was allowed to cool down to room temperature. The resulting mixture was filtered, the filter cake was washed with THF (3×5 mL). The filtrate was concentrated under reduced pressure. The crude product (130 mg) was purified by Prep-HPLC with the following conditions (Column: XBridge Shield RP18 OBD Column 30*150 mm, 51 μm; Mobile Phase A: Water(10 mmol/L NH₄HCO₃+0.1% NH₃.H₂O), Mobile Phase B: ACN; Flow rate: 60 mL/min; Gradient: 37% B to 48% B in 10 min; Wave Length: 254 nm/220 nm; RT1(min): 11.12) to afford 8-({4-[1-cyclopropyl-4-(trifluoromethyl)imidazo]-2-yl]-3-methoxyphenyl methyl)-2-(4-cyclopropyl-6-methoxypyrimidin-5-yl)pteridin-7-one (76.7 mg, 98.1% purity, 50.6%) as a red solid.

LC-MS (ESI) m/z 591.2 [M+H]

¹H NMR: (400 MHz, DMSO-d6) δ 9.38 (s, 1H), 8.70 (s, 1H), 8.50 (s, 1H), 7.84 (s, 1H), 7.24 (d, J=7.8 Hz, 1H), 7.19 (s, 1H), 6.92 (d, J=7.8 Hz, 1H), 5.49 (s, 2H), 3.83 (s, 3H), 3.71 (s, 3H), 3.45 (s, 1H), 3.32 (m, 1H), 1.80-1.73 (m, 1H), 1.06-0.99 (m, 2H), 0.80 (dt, J=7.8, 3.4 Hz, 2H), 0.73 (d, J=4.4 Hz, 4H).

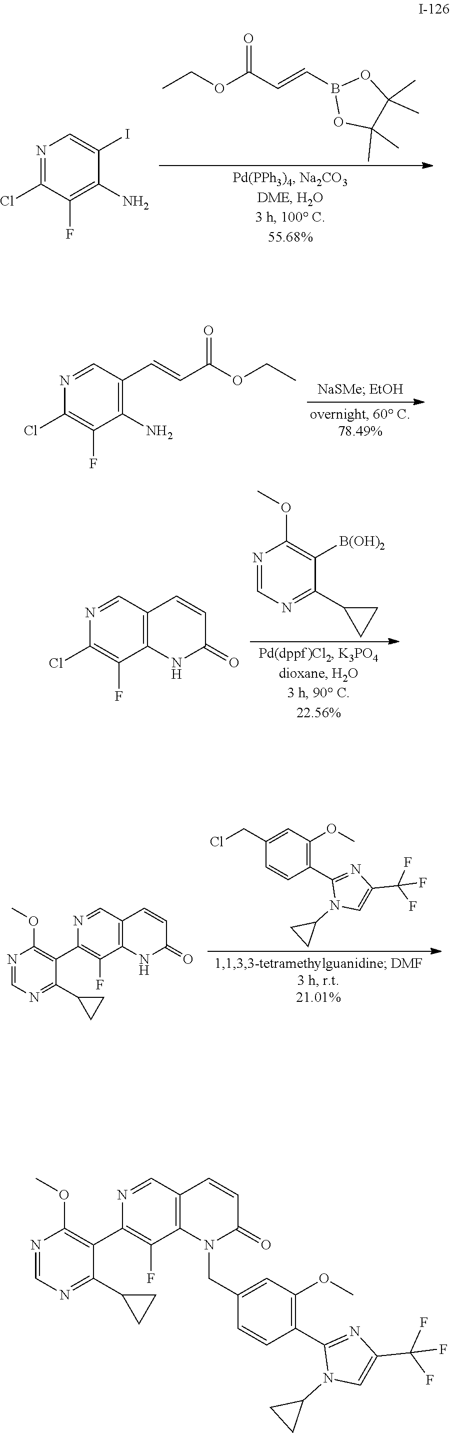

### Step 1: Ethyl (2E)-3-(4-Amino-6-Chloro-5-Fluoropyridin-3-Yl)Prop-2-Enoate

A solution of 2-chloro-3-fluoro-5-iodopyridin-4-amine (1 g, 3.670 mmol, 1 equiv), ethyl (2E)-3-(4,4,5,5-tetramethyl-1,3,2-dioxaborolan-2-yl)prop-2-enoate (1 g, 4.423 mmol, 1.21 equiv), Pd(PPh₃)₄ (0.4 g, 0.346 mmol, 0.09 equiv), Na₂CO₃ (0.8 g, 7.548 mmol, 2.06 equiv) and H₂O (3 mL, 166.528 mmol, 45.37 equiv) in DME (9 mL, 92.984 mmol, 25.33 equiv) was stirred for 3 h at 100° C. under nitrogen atmosphere. The mixture was allowed to cool down to room temperature. The residue was dissolved in water (50 mL). The resulting mixture was extracted with EtOAc (3×100 mL). The combined organic layers were washed with brine (3×50 mL), dried over anhydrous Na₂SO₄, filtered and concentrated under reduced pressure. The residue was purified by silica gel column chromatography, (eluting with PE/EA (1:1)) to afford ethyl (2E)-3-(4-amino-6-chloro-5-fluoropyridin-3-yl)prop-2-enoate (0.5 g, 55.68%) as an off-white solid.

LC-MS (ESI) m/z 245 [M+H]

### Step 2: 7-Chloro-8-Fluoro-1H-1,6-Naphthyridin-2-One

A solution of ethyl (2E)-3-(4-amino-6-chloro-5-fluoropyridin-3-yl)prop-2-enoate (273.9 mg, 1.120 mmol, 1 equiv) and Sodiummethanethiolate (196.15 mg, 2.800 mmol, 2.5 equiv) in EtOH (5 mL, 86.067 mmol, 76.88 equiv) was stirred for overnight at 60° C. under nitrogen atmosphere. The reaction was quenched by the addition of Water (10 mL) at 0° C. The mixture was neutralized to pH 7 with HCl (1M). The resulting mixture was extracted with EtOAc (3×50 mL). The combined organic layers were washed with brine (3×50 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 7-chloro-8-fluoro-1H-1,6-naphthyridin-2-one (174.5 mg, 78.49%) as an off-white solid.

LC-MS (ESI) m/z 199 [M+H]

### Step 3: 7-(4-Cyclopropyl-6-Methoxypyrimidin-5-Yl)-8-Fluoro-1H-1,6-Naphthyridin-2-One

To a stirred solution of 7-chloro-8-fluoro-1H-1,6-naphthyridin-2-one (82.6 mg, mmol, 1 equiv) and 4-cyclopropyl-6-methoxypyrimidin-5-ylboronic acid (96.8 mg, 0.499 mmol, 1.2 equiv) in dioxane (1.5 mL, 17.706 mmol, 42.57 equiv) were added K₃PO₄ (264.8 mg, 1.248 mmol, 3 equiv) and H₂O (0.3 mL, 16.653 mmol, 40.04 equiv) at room temperature under nitrogen atmosphere. To the above mixture was added Pd(dppf)C12 (60.8 mg, 0.083 mmol, 0.2 equiv) at room temperature. The resulting mixture was stirred for additional 3 h at 90° C. The resulting mixture was diluted with MeCN (5 mL). The residue was purified by reverse flash chromatography (C18 silica gel column, eluting with 0% to 100% MeCN/water (0.1% FA) in 10 min with UV detector (254 nm)) to afford 7-(4-cyclopropyl-6-methoxypyrimidin-5-yl)-8-fluoro-1H-1,6-naphthyridin-2-one (29.4 mg, 22.56%) as an orange solid.

LC-m/z 313 [M+H]

### Step 4: 1-({4-[1-Cyclopropyl-4-(Trifluoromethyl)Imidazol-2-Yl]-3-Methoxyphenyl}Methyl)-7-(4-Cyclopropyl-6-Methoxypyrimidin-5-Yl)-8-Fluoro-1,6-Naphthyridin-2-One

To a stirred solution of 7-(4-cyclopropyl-6-methoxypyrimidin-5-yl)-8-fluoro-1H-1,6-naphthyridin-2-one (29.4 mg, 0.094 mmol, 1 equiv) and 2-[4-(chloromethyl)-2-methoxyphenyl]-1-cyclopropyl-4-(trifluoromethyl)imidazole (31.1 mg, 0.094 mmol, 1 equiv) in DMF (0.6 mL, 7.753 mmol, 82.36 equiv) were added 1,1,3,3-tetramethylguanidine (32.5 mg, mmol, 3 equiv) at room temperature under nitrogen atmosphere. The resulting mixture was stirred for additional 3 h at room temperature. The resulting mixture was filtered, the filter cake was washed with MeCN (3×5 mL), filtered and concentrated under reduced pressure. The residue was purified by Prep-HPLC with the following conditions (Column: Sunfire prep C18 column mm, 51 μm; Mobile Phase A: Water(0.1% FA), Mobile Phase B: MeOH; Flow rate: 60 mL/min; Gradient: 53% B to 73% B in 10 min; Wave Length: 254 nm/220 nm; RT1(min): 10.3) to afford 1-({4-[1-cyclopropyl-4-(trifluoromethyl)imidazo]-2-yl]-3-methoxyphenyl methyl)-7-(4-cyclopropyl-6-methoxypyrimidin-5-yl)-8-fluoro-1,6-naphthyridin-2-one (12 mg, 21.01%) as an off-white solid.

LC-MS (ESI) m/z 607 [M+H]

¹H NMR (400 MHz, DMSO-d6) δ 8.92 (s, 1H), 8.67 (s, 1H), 8.23 (dd, J=9.6, 1.9 Hz, 1H), 7.84 (d, J=1.4 Hz, 1H), 7.25 (d, J=7.8 Hz, 1H), 7.05 (s, 1H), 6.96 (d, J=9.5 Hz, 1H), 6.63 (d, J=8.1 Hz, 1H), 5.66 (s, 2H), 3.75 (d, J=3.1 Hz, 6H), 1.60 (m, 1H), 1.06 (d, J=4.1 Hz, 1H), 1.00-0.82 (m, 2H), 0.75 (m, 1H), 0.72 (d, J=5.7 Hz, 4H).

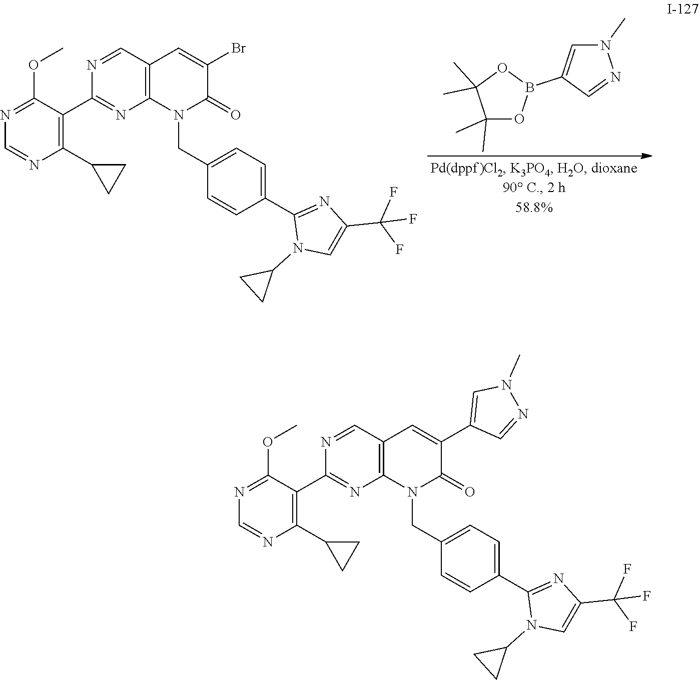

To a stirred mixture of 6-bromo-8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (100 mg, 0.157 mmol, 1 equiv) and 1-methyl-4-(4,4,5,5-tetramethyl-1,3,2-dioxaborolan-2-yl)pyrazole (48.9 mg, 0.235 mmol, 1.5 equiv) in H₂O (0.4 mL) and dioxane (1.6 mL) were added Pd(dppf)Cl₂ (22.9 mg, 0.031 mmol, 0.2 equiv) and K₃PO₄ (83.1 mg, 0.393 mmol, 2.5 equiv) at room temperature under nitrogen atmosphere. The resulting mixture was stirred for 2 h at 90° C. under nitrogen atmosphere. The mixture was allowed to cool down to room temperature. The resulting mixture was filtered, the filter cake was washed with acetonitrile (3×2 mL). The filtrate was concentrated under reduced pressure. The crude product (100 mg) was purified by Prep-HPLC with the following conditions (Column: XBridge Prep Phenyl OBD Column 19*250 mm, 51 μm; Mobile Phase A: Water(0.1% FA), Mobile Phase B: ACN; Flow rate: 60 mL/min; Gradient: 35% B to 54% B in 10 min; Wave Length: 254 nm/220 nm; RT1(min): 10.33) to afford 8-({4-[1-cyclopropyl-4-(trifluoromethyl)imidazo]-2-yl]phenyl}methyl)-2-(4-cyclopropyl-6-methoxypyrimidin-5-yl)-6-(1-methylpyrazol-4-yl)pyrido[2,3-d]pyrimidin-7-one (59.4 mg, 58.8%) as an off-white solid.

LC-MS (ESI) m/z 640.4 [M+H]

¹H NMR: (400 MHz, DMSO-d6) δ 9.25 (s, 1H), 8.69 (s, 1H), 8.50 (d, J=2.4 Hz, 2H), 8.15 (s, 1H), 7.90 (d, J=1.4 Hz, 1H), 7.84-7.78 (m, 2H), 7.42 (d, J=8.1 Hz, 2H), 5.68 (s, 2H), 3.91 (s, 3H), 3.84 (s, 3H), 3.68 (td, J=7.3, 3.7 Hz, 1H), 1.77 (td, J=8.1, 4.1 Hz, 1H), 1.05-1.00 (m, 2H), 0.95 (dd, J=7.3, 5.0 Hz, 2H), 0.94-0.85 (m, 2H), 0.78 (dq, J=6.9, 3.4 Hz, 2H).

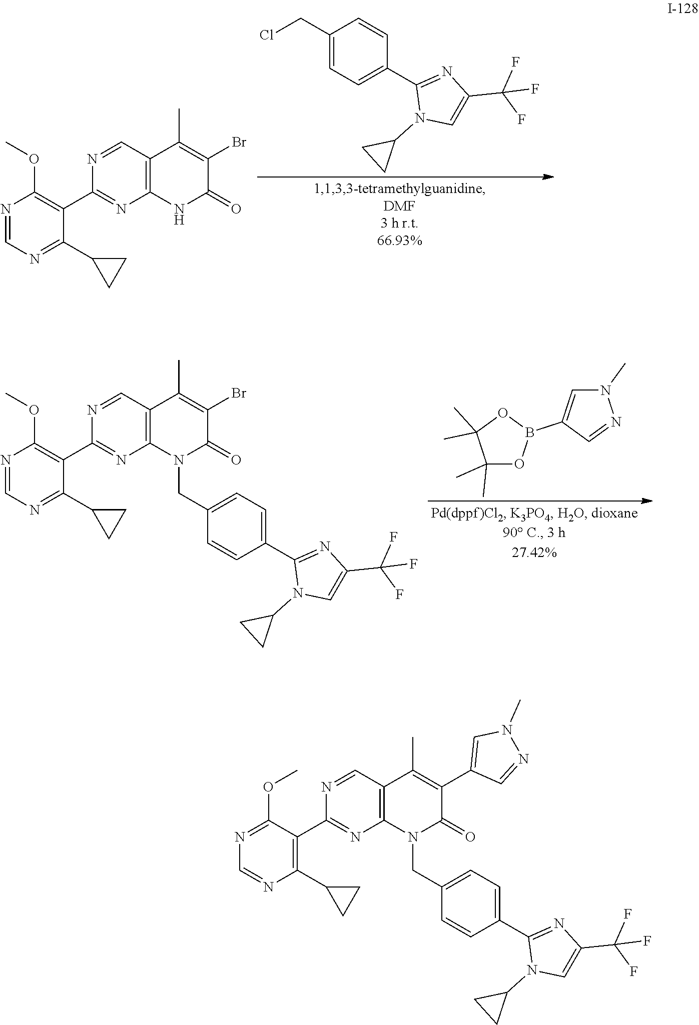

### Step 1: 6-Bromo-8-({4-[1-Cyclopropyl-4-(Trifluoromethyl)Imidazol-2-Yl]Phenyl}Methyl)-2-(4-Cyclopropyl-6-Methoxypyrimidin-5-371)-5-Methylpyrido[2,3-d]Pyrimidin-7-One

To a stirred solution of 6-bromo-2-(4-cyclopropyl-6-methoxypyrimidin-5-yl)-5-methyl-8H-pyrido[2,3-d]pyrimidin-7-one (265 mg, 0.683 mmol, 1 equiv) and 2-[4-(chloromethyl)phenyl]-1-cyclopropyl-4-(trifluoromethyl)imidazole (205.2 mg, 0.683 mmol, 1 equiv) in DMF (5 mL, 64.608 mmol, 94.65 equiv) were added 1,1,3,3-tetramethylguanidine (235.8 mg, 2.049 mmol, 3 equiv) at room temperature under nitrogen atmosphere. The resulting mixture was stirred for additional 3 h at room temperature. The resulting mixture was filtered, the filter cake was washed with MeCN (3×5 mL), filtered and concentrated under reduced pressure. The residue was purified by reverse flash chromatography (C18 silica gel column, eluting with 0% to 100% MeCN/water (0.1% FA) in 10 min with UV detector (254 nm)) to afford 6-bromo-8-({4-[1-cyclopropyl-4-(trifluoromethyl)imidazo]-2-yl]phenyl}methyl)-2-(4-cyclopropyl-6-methoxypyrimidin-5-yl)-5-methylpyrido[2,3-d]pyrimidin-7-one (298.1 mg, 66.93%) as a brown solid.

LC-MS (ESI) m/z 652[M+H]

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-5-Methyl-6-(1-Methylpyrazol-4-Yl)Pyrido[2,3-d]Pyrimidin-7-One

To a stirred solution of 6-bromo-8-({4-[1-cyclopropyl-4-(trifluoromethyl)imidazol-2-yl]phenyl}methyl)-2-(4-cyclopropyl-6-methoxypyrimidin-5-yl)-5-methylpyrido[2,3-d]pyrimidin-7-one (30 mg, 0.046 mmol, 1 equiv) and 1-methyl-4-(4,4,5,5-tetramethyl-1,3,2-dioxaborolan-2-yl)pyrazole (9.6 mg, 0.046 mmol, 1 equiv) in dioxane (0.6 mL, 7.082 mmol, 154.04 equiv) were added K₃PO₄ (29.3 mg, 0.138 mmol, 3 equiv) and H₂O (0.15 mL, 8.326 mmol, 181.09 equiv) at room temperature under nitrogen atmosphere. To the above mixture was added Pd(dppf)C12 (6.7 mg, 0.009 mmol, 0.2 equiv) at room temperature. The resulting mixture was stirred for additional 3 h at 90° C. The precipitated solids were collected by filtration and washed with MeOH (3×3 mL). The residue was purified by reverse flash chromatography (C18 silica gel column, eluting with 0% to 100% MeCN/water (0.1% FA) in 10 min with UV detector (254 nm)) to afford 8-({4-[1-cyclopropyl-4-(trifluoromethyl)imidazo]-2-yl]phenyl}methyl)-2-(4-cyclopropyl-6-methoxypyrimidin-5-yl)-5-methyl-6-(1-methylpyrazol-4-yl)pyrido[2,3-d]pyrimidin-7-one (8.3 mg, 27.42%) as an off-white solid.

LC-MS (ESI) m/z 654[M+H]

¹H NMR: (400 MHz, DMSO-d6) δ 9.42 (s, 1H), 8.69 (s, 1H), 8.06 (s, 1H), 7.90 (s, 1H), 7.80 (d, J=8.0 Hz, 2H), 7.70 (s, 1H), 7.40 (d, J=7.9 Hz, 2H), 5.63 (s, 2H), 3.91 (s, 3H), 3.84 (s, 3H), 3.68 (s, 2H), 2.65 (s, 3H), 1.74 (m, 1H), 1.02 (s, 2H), 1.03-0.92 (m, 2H), 0.89 (d, J=4.6 Hz, 2H), 0.77 (dd, J=7.9, 3.5 Hz, 2H).

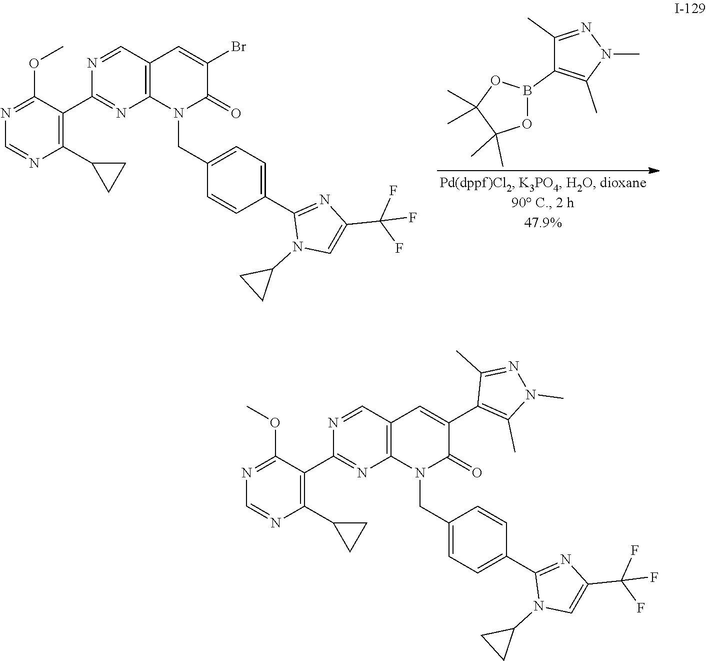

To a stirred mixture of 6-bromo-8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (70 mg, 0.110 mmol, 1 equiv) and 1,3,5-trimethyl-4-(4,4,5,5-tetramethyl-1,3,2-dioxaborolan-2-yl)pyrazole (38.8 mg, 0.165 mmol, 1.5 equiv) in H₂O (0.3 mL) and dioxane (1.2 mL) were added Pd(dppf)Cl₂ (16.0 mg, 0.022 mmol, 0.2 equiv) and K₃PO₄ (58.2 mg, 0.275 mmol, 2.5 equiv) at room temperature under nitrogen atmosphere. The resulting mixture was stirred for 2 h at 90° C. under nitrogen atmosphere. The mixture was allowed to cool down to room temperature. The resulting mixture was filtered, the filter cake was washed with acetonitrile (3×2 mL). The filtrate was concentrated under reduced pressure. The crude product (80 mg) was purified by Prep-HPLC with the following conditions (Column: XBridge Prep OBD C18 Column 30*150 mm, 51 μm; Mobile Phase A: Water(10 mmol/L NH₄HCO₃), Mobile Phase B: ACN; Flow rate: 60 mL/min; Gradient: 38% B to 56% B in 10 min; Wave Length: 254 nm/220 nm; RT1(min): 1) to afford 8-({4-[1-cyclopropyl-4-(trifluoromethyl)imidazo]-2-yl]phenyl methyl)-2-(4-cyclopropyl-6-methoxypyrimidin-5-yl)-6-(1,3,5-trimethylpyrazol-4-yl)pyrido[2,3-d]pyrimidin-7-one (35.3 mg, 47.9%) as an off-white solid.

LC-MS (ESI) m/z 668.3 [M+H]

¹H NMR (400 MHz, DMSO-d6) δ 9.28 (s, 1H), 8.70 (s, 1H), 8.01 (s, 1H), 7.91 (s, 1H), 7.81 (d, J=8.1 Hz, 2H), 7.43 (d, J=8.1 Hz, 2H), 5.64 (s, 2H), 3.84 (s, 3H), 3.71 (s, 4H), 2.17 (s, 3H), 2.08 (s, 3H), 1.76 (dd, J=8.2, 4.2 Hz, 1H), 1.03 (s, 2H), 0.98-0.92 (m, 2H), 0.89 (d, J=4.3 Hz, 1H), 0.78 (dd, J=7.8, 3.5 Hz, 2H).

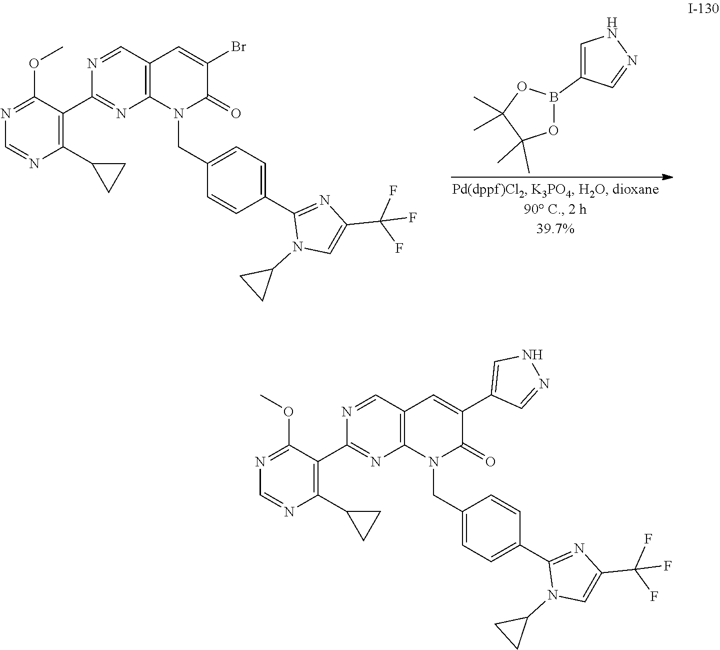 [SMILES: COc1ncnc(C2CC2)c1-c1ncc2cc(Br)c(=O)n(Cc3ccc(-c4nc(C(F)(F)F)cn4C4CC4)cc3)c2n1 ; CC1(C)OB(c2cn[nH]c2)OC1(C)C ; COc1ncnc(C2CC2)c1-c1ncc2cc(-c3cn[nH]c3)c(=O)n(Cc3ccc(-c4nc(C(F)(F)F)cn4C4CC4)cc3)c2n1]

To a stirred mixture of 6-bromo-8-({4-(1-cyclopropyl-4-yl)pyrido[2,3-d]pyrimidin-7-one (70 mg, 0.110 mmol, 1 equiv) and 4-(4,4,5,5-tetramethyl-1,3,2-dioxaborolan-2-yl)-1H-pyrazole (31.9 mg, 0.165 mmol, 1.5 equiv) in H₂O (0.3 mL) and dioxane (1.2 mL) were added Pd(dppf)C12 (16.05 mg, 0.022 mmol, 0.2 equiv) and K₃PO₄ (58.2 mg, 0.275 mmol, 2.5 equiv) at room temperature under nitrogen atmosphere. The resulting mixture was stirred for 2 h at 90° C. under nitrogen atmosphere. The mixture was allowed to cool down to room temperature. The resulting mixture was filtered, the filter cake was washed with acetonitrile (3×2 mL). The filtrate was concentrated under reduced pressure. The crude product (80 mg) was purified by Prep-HPLC with the following conditions (Column: Sunfire prep C18 column, 30*150 mm, 5 μm; Mobile Phase A: Water(0.1% FA), Mobile Phase B: ACN; Flow rate: 60 mL/min; Gradient: 36% B to 54 B in 10 min; Wave Length: 254 nm/220 nm; RT1(min): 12.02) to afford 8-({4-[1-cyclopropyl-4-(trifluoromethyl)imidazo]-2-yl]phenyl}methyl)-2-(4-cyclopropyl-6-methoxypyrimidin-5-yl)-6-(1H-pyrazol-4-yl)pyrido[2,3-d]pyrimidin-7-one (27.7 mg, 39.7%) as an off-white solid.

LC-MS (ESI) m/z 626.2 [M+H]

¹H NMR (400 MHz, DMSO-d6) δ 13.14 (s, 1H), 9.25 (s, 1H), 8.69 (s, 1H), 8.53 (s, 1H), 8.22 (s, 1H), 7.90 (d, J=1.4 Hz, 1H), 7.81 (d, J=8.2 Hz, 2H), 7.41 (d, J=8.2 Hz, 2H), 5.69 (s, 2H), 3.84 (s, 3H), 3.68 (s, 1H), 1.76 (dd, J=10.7, 6.2 Hz, 1H), 1.02 (d, J=5.3 Hz, 2H), 0.95 (dd, J=7.4, 5.0 Hz, 2H), 0.90 (s, 2H), 0.77 (dd, J=7.9, 3.3 Hz, 2H).

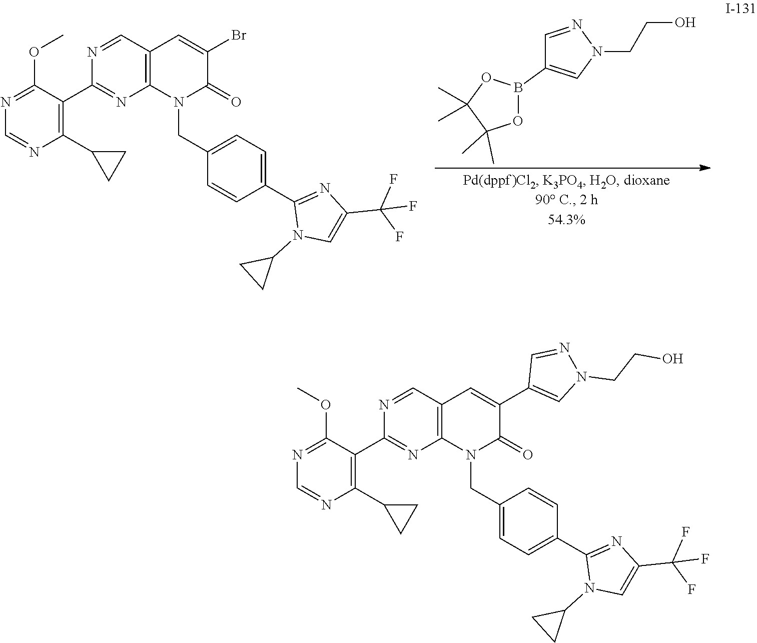 [SMILES: COc1ncnc(C2CC2)c1-c1ncc2cc(Br)c(=O)n(Cc3ccc(-c4nc(C(F)(F)F)cn4C4CC4)cc3)c2n1 ; CC1(C)OB(c2cnn(CCO)c2)OC1(C)C ; COc1ncnc(C2CC2)c1-c1ncc2cc(-c3cnn(CCO)c3)c(=O)n(Cc3ccc(-c4nc(C(F)(F)F)cn4C4CC4)cc3)c2n1]

To a stirred mixture of 6-bromo-8-({4-(1-cyclopropyl-4-yl)pyrido[2,3-d]pyrimidin-7-one (80 mg, 0.125 mmol, 1 equiv) and 2-[4-(4,4,5,5-tetramethyl-1,3,2-dioxaborolan-2-yl)pyrazol-1-yl]ethanol (44.7 mg, 0.188 mmol, 1.5 equiv) in H₂O (0.3 mL) and dioxane (1.2 mL) were added K₃PO₄ (66.5 mg, 0.313 mmol, 2.5 equiv) and Pd(dppf)C12 (18.34 mg, 0.025 mmol, 0.2 equiv) at room temperature under nitrogen atmosphere. The resulting mixture was stirred for 2 h at 90° C. under nitrogen atmosphere. The mixture was allowed to cool down to room temperature. The resulting mixture was filtered, the filter cake was washed with acetonitrile (3×2 mL). The filtrate was concentrated under reduced pressure. The crude product (90 mg) was purified by Prep-HPLC with the following conditions (Column: YMC-Actus Triart C18 ExRS 30*150 mm, 51 μm; Mobile Phase A: Water(10 mmol/L NH₄HCO₃), Mobile Phase B: ACN; Flow rate: 60 mL/min; Gradient: 37% B to 54.5% B in 10 min; Wave Length: 254 nm/220 nm; RT1(min): 10.98) to afford 8-({4-[1-cyclopropyl-4-(trifluoromethyl)imidazo]-2-yl]phenyl}methyl)-2-(4-cyclopropyl-6-methoxypyrimidin-5-yl)-6-{1-[2-(dimethylamino)ethyl]pyrazol-4-yl}pyrido[2,3-d]pyrimidin-7-one (48 mg, 54.32%) as an off-white solid.

LC-MS (ESI) m/z 670.2 [M+H]

¹H NMR: (400 MHz, DMSO-d6) δ 9.25 (s, 1H), 8.69 (s, 1H), 8.52 (d, J=3.5 Hz, 2H), 8.18 (s, 1H), 7.93-7.88 (m, 1H), 7.81 (d, J=8.2 Hz, 2H), 7.42 (d, J=8.1 Hz, 2H), 5.69 (s, 2H), 4.93 (t, J=5.3 Hz, 1H), 4.21 (t, J=5.4 Hz, 2H), 3.84 (s, 3H), 3.76 (q, J=5.5 Hz, 2H), 3.68 (s, 1H), 1.77 (dd, J=7.9, 3.4 Hz, 1H), 1.02 (s, 2H), 0.96 (d, J=7.0 Hz, 1H), 0.92 (d, J=13.8 Hz, 1H), 0.78 (dd, J=8.0, 3.6 Hz, 2H).

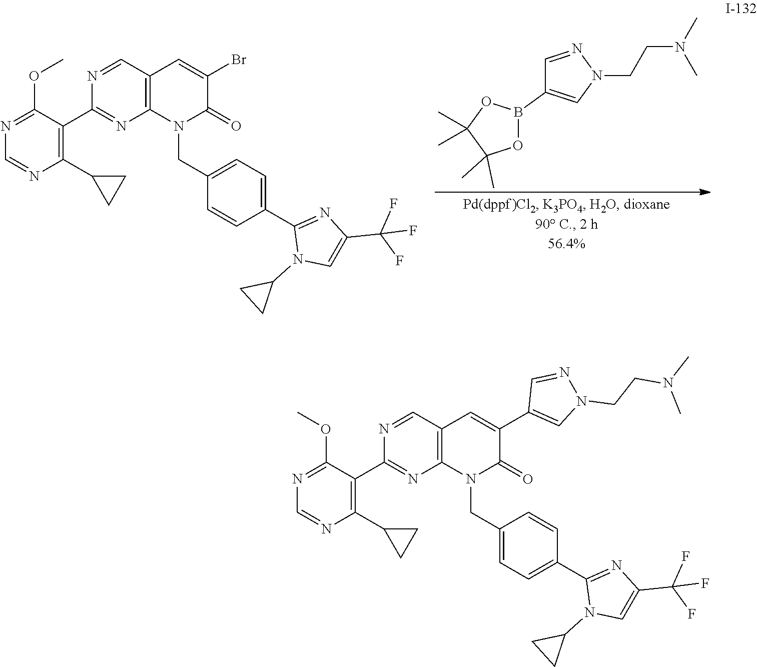

To a stirred mixture of 6-bromo-8-({4-(1-cyclopropyl-4-yl)pyrido[2,3-d]pyrimidin-7-one (70 mg, 0.110 mmol, 1 equiv) and dimethyl({2-[4-(4,4,5,5-tetramethyl-1,3,2-dioxaborolan-2-yl)pyrazol-1-yl]ethyl})amine (43.6 mg, 0.165 mmol, 1.5 equiv) in H₂O (0.3 mL) and dioxane (1.2 mL) were added Pd(dppf)C12 (16.0 mg, 0.022 mmol, 0.2 equiv) and K₃PO₄ (58.2 mg, 0.275 mmol, 2.5 equiv) at room temperature under nitrogen atmosphere. The resulting mixture was stirred for 2 h at under nitrogen atmosphere. The mixture was allowed to cool down to room temperature. The resulting mixture was filtered, the filter cake was washed with acetonitrile (3×2 mL). The filtrate was concentrated under reduced pressure. The crude product (80 mg) was purified by Prep-HPLC with the following conditions (Column: XBridge Prep Phenyl OBD Column 19*250 mm, 51 μm; Mobile Phase A: Water(10 mmol/L NH₄HCO₃+0.1% NH₃.H₂O), Mobile Phase B: ACN; Flow rate: mL/min; Gradient: 35% B to 55% B in 10 min; Wave Length: 254 nm/220 nm; RT 1 (min): 9.08) to afford 8-({4-[1-cyclopropyl-4-(trifluoromethyl)imidazo]-2-yl]phenyl methyl)-2-(4-cyclopropyl-6-methoxypyrimidin-5-yl)-6-{1-[2-(dimethylamino)ethyl]pyrazol-4-yl}pyrido[2,3-d]pyrimidin-7-one (44 mg, 56.4%) as an off-white solid.

LC-MS (ESI) m/z 697.3 [M+H]

¹H NMR (400 MHz, DMSO-d6)_{δ 9.25} (s, 1H), 8.69 (s, 1H), 8.52 (d, J=15.9 Hz, 2H), 8.16 (s, 1H), 7.90 (d, J=1.4 Hz, 1H), 7.85-7.78 (m, 2H), 7.42 (d, J=8.2 Hz, 2H), 5.69 (s, 2H), 4.26 (t, J=6.4 Hz, 2H), 3.84 (s, 3H), 3.73-3.63 (m, 1H), 2.67 (t, J=6.4 Hz, 2H), 2.17 (s, 6H), 1.77 (td, J=8.2, 4.2 Hz, 1H), 1.05-0.99 (m, 2H), 0.95 (dd, J=7.4, 5.1 Hz, 2H), 0.95-0.85 (m, 2H), 0.82-0.74 (m, 2H).

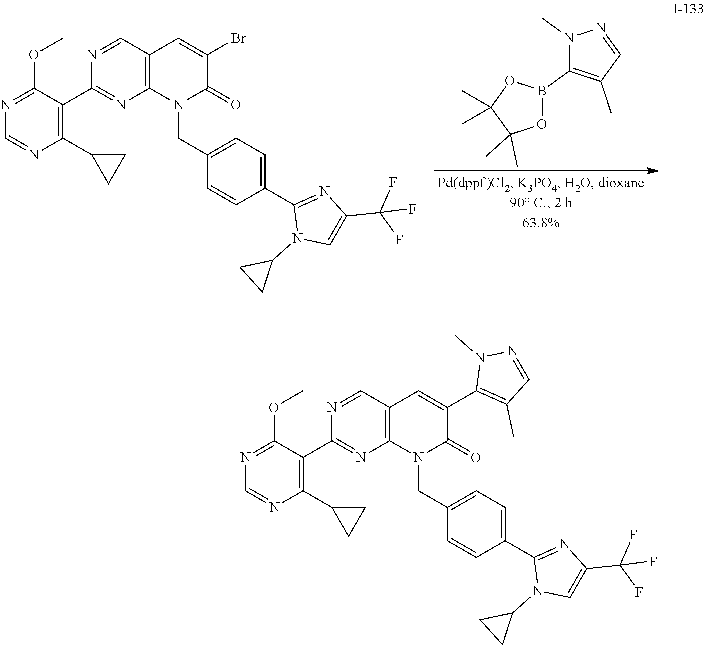 [SMILES: COc1ncnc(C2CC2)c1-c1ncc2cc(Br)c(=O)n(Cc3ccc(-c4nc(C(F)(F)F)cn4C4CC4)cc3)c2n1 ; Cc1cnn(C)c1B1OC(C)(C)C(C)(C)O1 ; COc1ncnc(C2CC2)c1-c1ncc2cc(-c3c(C)cnn3C)c(=O)n(Cc3ccc(-c4nc(C(F)(F)F)cn4C4CC4)cc3)c2n1]

To a stirred mixture of 6-bromo-8-({4-(1-cyclopropyl-4-yl)pyrido[2,3-d]pyrimidin-7-one (70 mg, 0.110 mmol, 1 equiv) and 1,4-dimethyl-5-(4,4,5,5-tetramethyl-1,3,2-dioxaborolan-2-yl)pyrazole (36.5 mg, 0.165 mmol, 1.5 equiv) in H₂O (0.3 mL) and dioxane (1.2 mL) were added Pd(dppf)Cl₂ (16.0 mg, 0.022 mmol, 0.2 equiv) and K₃PO₄ (58.2 mg, 0.275 mmol, 2.5 equiv) at room temperature under nitrogen atmosphere. The resulting mixture was stirred for 2 h at 90° C. under nitrogen atmosphere. The mixture was allowed to cool down to room temperature. The resulting mixture was filtered, the filter cake was washed with acetonitrile (3×2 mL). The filtrate was concentrated under reduced pressure. The crude product (80 mg) was purified by Prep-HPLC with the following conditions (Column: XBridge Prep Phenyl OBD Column 19*250 mm, 51 μm; Mobile Phase A: Water(0.1% FA), Mobile Phase B: ACN; Flow rate: 60 mL/min; Gradient: 35% B to 55% B in 10 min; Wave Length: 254 nm/220 nm; RT1(min): 10.49) to afford 8-({4-[1-cyclopropyl-4-(trifluoromethyl)imidazo]-2-yl]phenyl}methyl)-2-(4-cyclopropyl-6-methoxypyrimidin-5-yl)-6-(2,4-dimethylpyrazol-3-yl)pyrido[2,3-d]pyrimidin-7-one (46 mg, 63.8%) as an off-white solid.

LC-MS (ESI) m/z 654.2 [M+H]

¹H NMR (400 MHz, DMSO-d6) δ 9.35 (s, 1H), 8.71 (s, 1H), 8.29 (s, 1H), 7.91 (d, J=1.5 Hz, 1H), 7.82 (d, J=8.3 Hz, 2H), 7.45 (d, J=8.1 Hz, 2H), 7.36 (s, 1H), 5.65 (s, 2H), 3.84 (s, 3H), 3.71 (d, J=4.2 Hz, 1H), 3.69 (s, 3H), 1.99 (s, 3H), 1.77 (dd, J=8.2, 3.8 Hz, 1H), 1.04 (s, 2H), 0.97 (d, J=13.9 Hz, 2H), 0.93 (d, J=30.5 Hz, 2H), 0.79 (dd, J=7.9, 3.4 Hz, 2H).

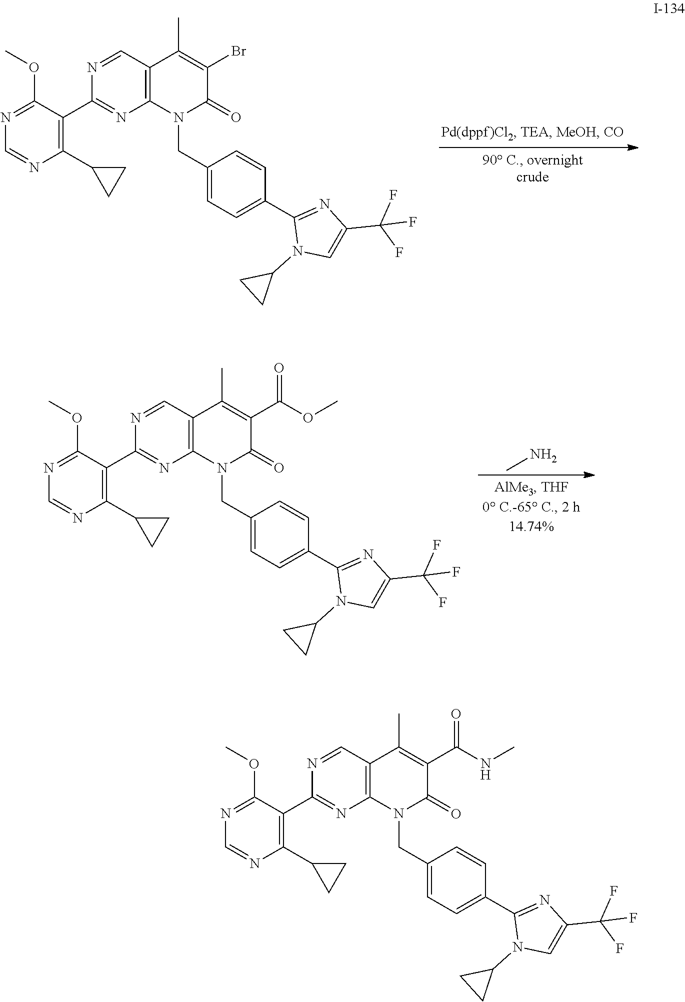

### Step 1: Methyl 8-({4-[1-Cyclopropyl-4-(Trifluoromethyl)Imidazol-2-Yl]Phenyl}Methyl)-2-(4-Cyclopropyl-6-Methoxypyrimidin-5-Yl)-5-Methyl-7-Oxopyrido[2,3-Cl]Pyrimidine-6-Carboxylate

To a stirred solution of 6-bromo-8-({4-[1-cyclopropyl-4-(trifluoromethyl)imidazol-2-yl]phenyl methyl)-2-(4-cyclopropyl-6-methoxypyrimidin-5-yl)-5-methylpyrido[2,3-d]pyrimidin-7-one (50 mg, 0.077 mmol, 1 equiv) and Pd(dppf)Cl₂ (11.2 mg, mmol, 0.2 equiv) in MeOH (5 mL, 123.494 mmol, 1611.54 equiv) were added TEA (23.2 mg, 0.231 mmol, 3 equiv) at room temperature under carbon monoxide atmosphere. The resulting mixture was stirred for additional overnight at 90° C. The resulting mixture was filtered, the filter cake was washed with MeCN (3×5 mL), the filtrate was concentrated under reduced pressure. The residue was purified by reverse flash chromatography (C18 silica gel column, eluting with 0% to 100% MeCN/water (0.1% FA) in 10 min with UV detector (254 nm)) to afford methyl 8-({4-[1-cyclopropyl-4-(trifluoromethyl)imidazol-2-yl]phenyl methyl)-2-(4-cyclopropyl-6-methoxypyrimidin-5-yl)-5-methyl-7-oxopyrido[2,3-d]pyrimidine-6-carboxylate (54.2 mg, crude) as a brown solid.

LC-MS (ESI) m/z 632[M+H]

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-N,5-Dimethyl-7-Oxopyrido[2,3-Cl]Pyrimidine-6-Carboxamide

To a stirred solution of Methylamine, 2M in THF (0.13 mL, 0.258 mmol, 3 equiv) in THF (0.6 mL, 7.406 mmol, 86.30 equiv) were added AlMe₃ (0.13 mL, 0.258 mmol, 3 equiv, 2M in THF) at 0° C. under nitrogen atmosphere. The resulting mixture was stirred for additional 0.5 h at 0° C. To the above mixture was added methyl 8-({4-[1-cyclopropyl-4-(trifluoromethyl)imidazo]-2-yl]phenyl methyl)-2-(4-cyclopropyl-6-methoxypyrimidin-5-yl)-5-methyl-7-oxopyrido[2,3-d]pyrimidine-6-carboxylate (54.2 mg, 0.086 mmol, 1 equiv) at 0° C. The resulting mixture was stirred for additional 2 h at 65° C. The reaction was quenched by the addition of Water (5 mL) at 0° C. The resulting mixture was extracted with EtOAc (3×10 mL). The combined organic layers were washed with brine (3×5 mL), dried over anhydrous Na₂SO₄, filtered and concentrated under reduced pressure. The crude product (60 mg) was purified by Prep-HPLC with the following conditions (Column: Sunfire prep C18 column, 30*150 mm, 5 μm; Mobile Phase A: Water(0.1% FA), Mobile Phase B: ACN; Flow rate: 60 mL/min; Gradient: isocratic 30% B t50% B in 10 min; Wave Length: 254 nm/220 nm; RT1(min): 11.27) to afford 8-({4-[1-cyclopropyl-4-(trifluoromethyl)imidazo]-2-yl]phenyl}methyl)-2-(4-cyclopropyl-6-methoxypyrimidin-5-yl)-N,5-dimethyl-7-oxopyrido[2,3-d]pyrimidine-6-carboxamide (8.1 mg, 14.74%) as an off-white solid.

LC-MS (ESI) m/z 631[M+H]

¹H NMR (400 MHz, DMSO-d6) δ 9.41 (s, 1H), 8.70 (s, 1H), 8.31 (s, 1H), 7.91 (s, 1H), 7.81 (d, J=8.2 Hz, 2H), 7.41 (d, J=8.3 Hz, 2H), 5.56 (s, 2H), 3.83 (s, 3H), 3.69 (m, 1H), 2.78 (d, J=4.6 Hz, 3H), 2.49 (s, 3H), 1.70 (m, 1H), 1.02 (m, 2H), 0.96 (m, 2H), 0.91 (dd, J=4.3 Hz, 2H), 0.77 (dd, J=7.9, 3.4 Hz, 2H).

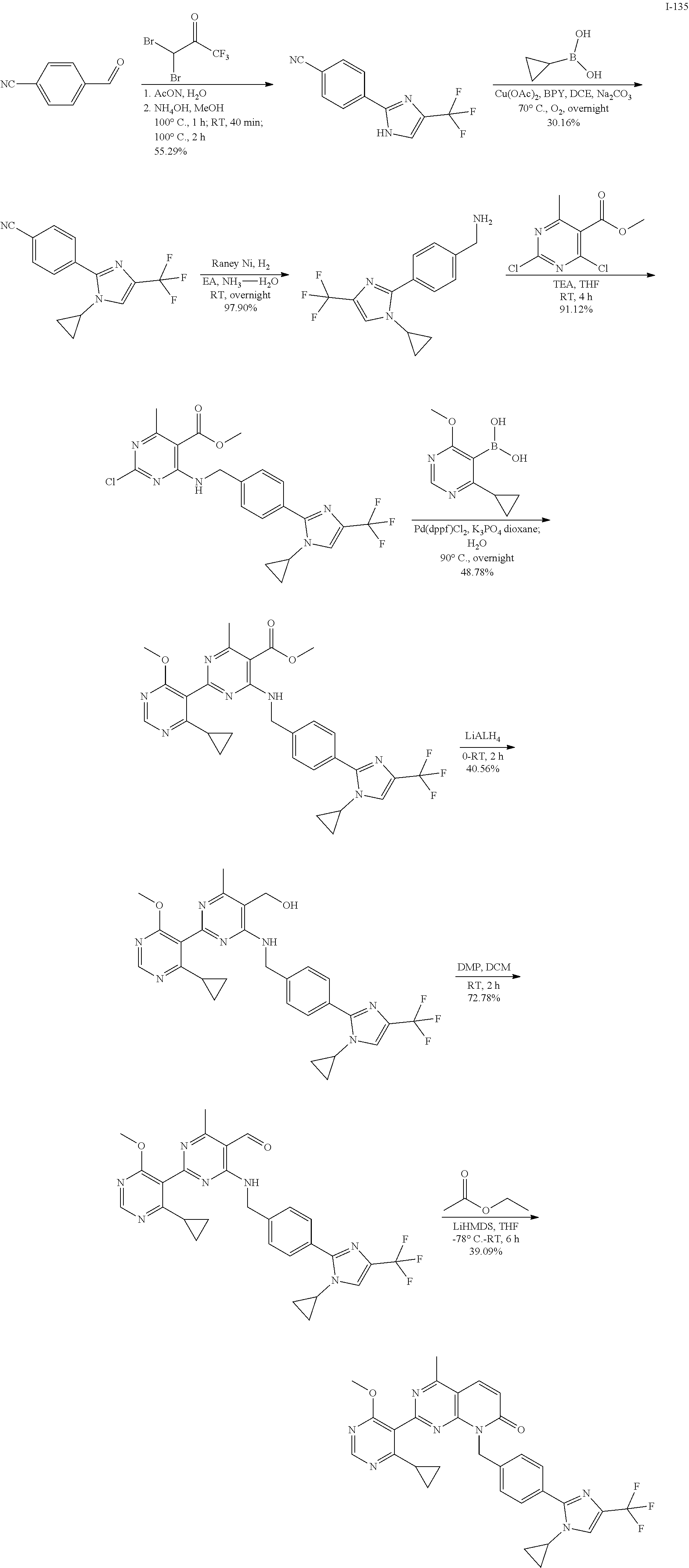

Step 1: 4-[4-(trifluoromethyl)-1H-imidazol-2-yl]benzonitrile To a stirred solution of 3,3-dibromo-1,1,1-trifluoropropan-2-one (23.05 g, 85.410 mmol, 1.12 equiv) in H₂O (20 mL) was added AcONa (7.01 g, 85.410 mmol, 1.12 equiv) at room temperature under nitrogen atmosphere. The resulting mixture was stirred for 1 h at 100° C. under nitrogen atmosphere, then cooled to room temperature. To the above mixture was added 4-formylbenzonitrile (10 g, 76.259 mmol, 1.00 equiv) in a mixture of MeOH (152 mL) and NH₄OH (36 mL). The resulting mixture was stirred for 40 min at room temperature. Then, the reaction was warmed to 100° C. and stirred for 2h. The reaction was quenched by the addition of Water/Ice (150 mL) at room temperature. The resulting mixture was extracted with EtOAc (3×100 mL). The combined organic layers were washed with water (3×150 mL), dried over anhydrous Na₂SO₄, filtered and concentrated under reduced pressure. The residue was purified by silica gel column chromatography, (eluting with PE/EA (1:1)) to afford 4-[4-(trifluoromethyl)-1H-imidazol-2-yl]benzonitrile (10 g, 55.29%) as a yellow solid.

LC-MS (ESI) m/z 238.05 [M+H]

### Step 2: 4-[1-Cyclopropyl-4-(Trifluoromethyl)Imidazol-2-Yl]Benzonitrile

To a stirred solution of 4-[4-(trifluoromethyl)-1H-imidazol-2-yl]benzonitrile (10 g, 42.161 mmol, 1 equiv) and cyclopropylboronic acid (7.24 g, 84.322 mmol, 2 equiv) in DCE (200 mL) were added Cu(OAc)₂ (3.83 g, 21.081 mmol, 0.5 equiv), Na₂CO₃ (17.87 g, 168.644 mmol, 4 equiv) and bipyridyl (6.59 g, 42.161 mmol, 1 equiv) at room temperature. The resulting mixture was stirred for overnight at 70° C. under 02 atmosphere. The resulting mixture was diluted with water (150 mL). The resulting mixture was extracted with CH₂Cl₂ (3×70 mL). The combined organic layers were washed with brine (2×150 mL), dried over anhydrous Na₂SO₄, filtered and concentrated under reduced pressure. The residue was purified by silica gel column chromatography, (eluting with PE/EA (2:1)) to afford 4-[1-cyclopropyl-4-(trifluoromethyl)imidazol-2-yl]benzonitrile (3.526 g, 30.16%) as an off-white solid.

LC-MS (ESI) m/z 278.08 [M+H]

### Step 3: 1-{4-[1-Cyclopropyl-4-(Trifluoromethyl)Imidazol-2-Yl]Phenyl}Methanamine

To a stirred mixture of 4-[1-cyclopropyl-4-(trifluoromethyl)imidazol-2-yl]benzonitrile (3.524 g, 12.711 mmol, 1 equiv) in NH 4 OH (50 mL) and EA (50 mL) was added Raney Ni (3.6 g, 42.019 mmol, 3.31 equiv) under nitrogen atmosphere in a 250 mL round-bottom flask. The mixture was hydrogenated at room temperature for overnight under hydrogen atmosphere using a hydrogen balloon, filtered through a Celite pad and concentrated under reduced pressure. The residue was purified by reversed-phase flash chromatography (C18 silica gel column, eluting with 0% to 100% MeCN/water (0.1% FA) in 20 min with UV detector (254 nm)) to afford 1-{4-[1-cyclopropyl-4-(trifluoromethyl)imidazo]-2-yl]phenyl}methanamine (3.5 g, 97.90%) as a greenish solid.

LC-MS (ESI) m/z 282.11 [M+H]

### Step 4: Methyl 2-Chloro-4-1({4-[1-Cyclopropyl-4-(Trifluoromethyl)Imidazol-2-Yl]Phenyl}Methyl)Amino]-6-Methylpyrimidine-5-Carboxylate

To a stirred solution of methyl 2,4-dichloro-6-methylpyrimidine-5-carboxylate (864.4 mg, 3.910 mmol, 2 equiv) and 1-{4-[1-cyclopropyl-4-(trifluoromethyl)imidazo]-2-yl]phenyl}methanamine (550 mg, 1.955 mmol, 1.00 equiv) in THF (16 mL) was added TEA (593.6 mg, 5.865 mmol, 3 equiv) at room temperature. The resulting mixture was stirred for 4 h at room temperature. The mixture was purified by silica gel column chromatography, (eluting with PE/EA (2:1)) to afford methyl 2-chloro-4-[({4-[1-cyclopropyl-4-(trifluoromethyl)imidazol-2-yl]phenyl}methyl)amino]-6-methylpyrimidine-5-carboxylate (830 mg, 91.12%) as a light yellow oil.

LC-MS (ESI) m/z 466.12 [M+H]

### Step 5: Methyl 4′-Cyclopropyl-4-1({4-[1-Cyclopropyl-4-(Trifluoromethyl)Imidazol-2-Yl]Phenyl}Methyl)Amino]-6′-Methoxy-6-Methyl-12,5′-Bipyrimidine]-5-Carboxylate

To a stirred solution of methyl 2-chloro-44({4-[1-cyclopropyl-4-(trifluoromethyl)imidazo]-2-yl]phenyl methyl)amino]-6-methylpyrimidine-5-carboxylate (730 mg, 1.567 mmol, 1 equiv) and 4-cyclopropyl-6-methoxypyrimidin-5-ylboronic acid (456.0 mg, 2.350 mmol, 1.5 equiv) in dioxane (12 mL) and H₂O (2.5 mL) were added K₃PO₄ (831.6 mg, 3.917 mmol, 2.5 equiv) and Pd(dppf)C12 (229.3 mg, 0.313 mmol, 0.2 equiv) at room temperature. The resulting mixture was stirred for overnight at 90° C. under nitrogen atmosphere. The resulting mixture was filtered, the filter cake was washed with THF (2×5 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford methyl 4′-cyclopropyl-44({4-[1-cyclopropyl-4-(trifluoromethyl)imidazo]-2-yl]phenyl methyl)amino]-6′-methoxy-6-methyl-[2, 5′-bipyrimidine]-5-carboxylate (443 mg, 48.78%) as a brown oil.

LC-MS (ESI) m/z 580.22 [M+H]

### Step 6: {4′-Cyclopropyl-4-1({4-[1-Cyclopropyl-4-(Trifluoromethyl)Imidazol-2-Yl]Phenyl}Methyl)Aminol-6′-Methoxy-6-Methyl-12,5′-Bipyrimidin]-5-Yl}Methanol

To a stirred solution of methyl 4′-cyclopropyl-44({4-[1-cyclopropyl-4-(trifluoromethyl)imidazo]-2-yl]phenyl methyl)amino]-6′-methoxy-6-methyl-[2,5′-bipyrimidine]-5-carboxylate (443 mg, 0.764 mmol, 1 equiv) in THF (10 mL) was added LiAlH₄ (145.03 mg, 3.820 mmol, 5 equiv) at 0° C. under nitrogen atmosphere. The resulting mixture was stirred for 2h at room temperature under nitrogen atmosphere. The reaction was quenched with Na₂SO₄.10H₂O at 0° C. The resulting mixture was filtered, the filter cake was washed with THF (3×15 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford {4′-cyclopropyl-4-[({4-[1-cyclopropyl-4-(trifluoromethyl)imidazo]-2-yl]phenyl}methyl)amino]-6′-methoxy-6-methyl-[2,5′-bipyrimidin]-(171 mg, 40.56%) as a colorless oil.

LC-MS (ESI) m/z 552.23 [M+H]

### Step 7: 4′-Cyclopropyl-44({4-[1-Cyclopropyl-4-(Trifluoromethyl)Imidazol-2-Yl]Phenyl}Methyl)Aminol-6′-Methoxy-6-Methyl-12,5′-Bipyrimidine]-5-Carbaldehyde

To a stirred solution of {4′-cyclopropyl-44({4-[1-cyclopropyl-4-(trifluoromethyl)imidazo]-2-yl]phenyl}methyl)amino]-6′-methoxy-6-methyl-[2,5′-bipyrimidin]-(171 mg, 0.310 mmol, 1 equiv) in DCM (4 mL) was added DMP (263.0 mg, 0.620 mmol, 2 equiv) at room temperature. The resulting mixture was stirred for 2 h at room temperature. The resulting mixture was filtered, the filter cake was washed with THF (3×10 mL). The filtrate was concentrated under reduced pressure. The residue was purified by silica gel column chromatography, (eluting with PE/EA (3:1)) to afford 4′-cyclopropyl-44({4-[1-cyclopropyl-4-(trifluoromethyl)imidazo]-2-yl]phenyl}methyl)amino]-6′-methoxy-6-methyl-[2,5′-bipyrimidine]-aldehyde (124 mg, 72.78%) as a light yellow solid.

LC-MS (ESI) m/z 550.21 [M+H]

### Step 8: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-4-Methylpyrido[2,3-d]Pyrimidin-7-One

To a stirred solution of ethyl acetate (80.2 mg, 0.910 mmol, 10 equiv) in THF (2 mL) was added LiHMDS (152.2 mg, 0.910 mmol, 10 equiv) at −78° C. under nitrogen atmosphere. The resulting mixture was stirred for 10 min at −78° C. under nitrogen atmosphere. To the above mixture was added a solution of 4′-cyclopropyl-4-[({4-[1-cyclopropyl-4-(trifluoromethyl)imidazol-2-yl]phenyl}methyl)amino]-6′-methoxy-6-methyl-[2,5′-bipyrimidine]-aldehyde (50 mg, 0.091 mmol, 1 equiv) in THF at −78° C. The resulting mixture was stirred for additional 6 h at room temperature. The reaction was quenched by the addition of Water (1 mL) at room temperature. The resulting mixture was concentrated under reduced pressure. The residue was purified by reversed-phase flash chromatography (C18 silica gel column, eluting with 0% to 100% MeCN/water (0.1% FA) in 10 min with UV detector (254 nm)) to the crude product. The crude product was purified by Prep-HPLC with the following conditions (Column: Sunfire prep C18 column, 30*150 mm, 5 μm; Mobile Phase A: Water(0.05% TFA), Mobile Phase B: ACN; Flow rate: 60 mL/min; Gradient: isocratic 45% B to 63% B in 10 min; Wave Length: 254 nm/220 nm; RT1(min): 9.22) to afford 8-({4-[1-cyclopropyl-4-(trifluoromethyl)imidazo]-2-yl]phenyl}methyl)-2-(4-cyclopropyl-6-methoxypyrimidin-5-yl)-4-methylpyrido[2,3-d]pyrimidin-7-one (20.4 mg, 39.09%) as an off-white solid.

LC-MS (ESI) m/z 574.30 [M+H]

¹H NMR (400 MHz, DMSO-d6) δ 8.68 (s, 1H), 8.31 (d, J=9.7 Hz, 1H), 7.90 (s, 1H), 7.80 (d, J=8.0 Hz, 2H), 7.37 (d, J=8.1 Hz, 2H), 6.88 (d, J=9.7 Hz, 1H), 5.58 (s, 2H), 3.82 (s, 3H), 3.74-3.64 (m, 1H), 2.81 (s, 3H), 1.75-1.64 (m, 1H), 1.40 (s, OH), 1.04-0.93 (m, 4H),−0.86 (m, 2H), 0.80-0.71 (m, 2H).

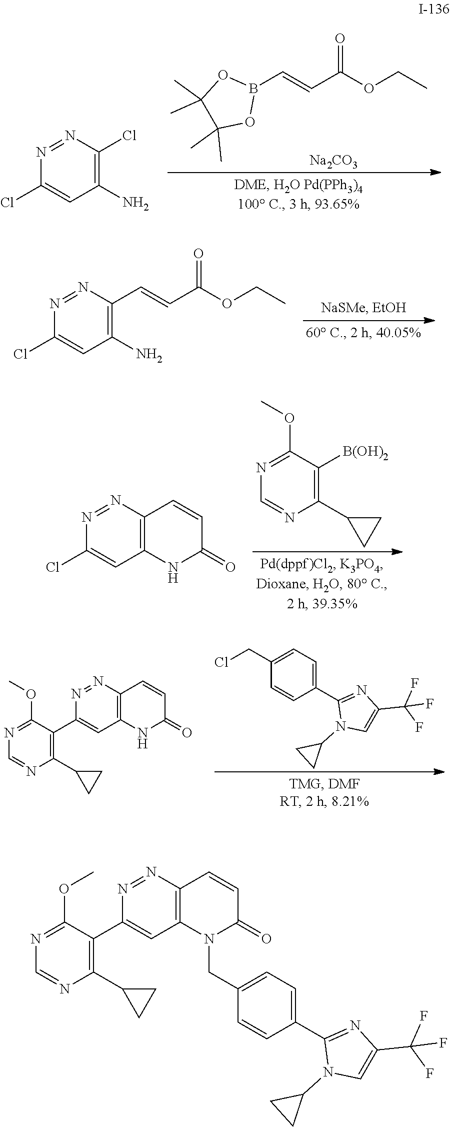

### Step 1: Ethyl (2E)-3-(4-Amino-6-Chloropyridazin-3-Yl)Prop-2-Enoate

A solution of 3,6-dichloropyridazin-4-amine (500 mg, 3.049 mmol, 1 equiv), ethyl (2E)-3-(4,4,5,5-tetramethyl-1,3,2-dioxaborolan-2-yl)prop-2-enoate (903.00 mg, 3.994 mmol, 1.31 equiv), tetrakis(triphenylphosphine)palladium(0) (352.34 mg, 0.305 mmol, 0.10 equiv), Na₂CO₃ (649.54 mg, 6.128 mmol, 2.01 equiv) and H₂O (5.00 mL, 277.550 mmol, 91.03 equiv) in DME (15 mL) was stirred for 3h at 100° C. under nitrogen atmosphere. The mixture was allowed to cool down to room temperature. The resulting mixture was concentrated under reduced pressure. The residue was purified by reverse flash chromatography (C18 silica gel column, eluting with 10% to 50% MeCN/water (0.1% FA) in 10 min with UV detector (254 nm)) to afford ethyl (2E)-3-(4-amino-6-chloropyridazin-3-yl)prop-2-enoate (650 mg, 93.65%) as a yellow solid.

LC-MS (ESI) m/z 227 [M+H].

### Step 2: 3-Chloro-511-Pyrido[3,2-c]Pyridazin-6-One

A solution of ethyl (2E)-3-(4-amino-6-chloropyridazin-3-yl)prop-2-enoate (720 mg, 3.163 mmol, 1 equiv) and sodium methanethiolate (432.21 mg, 6.168 mmol, 1.95 equiv) in EtOH (30 mL) was stirred for 2 h at 60° C. under nitrogen atmosphere. The mixture was allowed to cool down to room temperature. The resulting mixture was diluted with water (10 mL). The mixture was neutralized to pH 7 with 1M HCl. The precipitated solids were collected by filtration and washed with EtOH (2×10 mL). This resulted in 3-chloro-5H-pyrido[3,2-c]pyridazin-6-one (230 mg, 40.05%) as an off-white solid.

LC-MS (ESI) m/z 182 [M+H].

### Step 3: 3-(4-Cyclopropyl-6-Methoxypyrimidin-5-Yl)-511-Pyrido[3,2-c]Pyridazin-6-One

A solution of 3-chloro-5H-pyrido[3,2-c]pyridazin-6-one (100 mg, 0.551 mmol, 1 equiv), 4-cyclopropyl-6-methoxypyrimidin-5-ylboronic acid (119.66 mg, 0.617 mmol, 1.12 equiv), Pd(dppf)Cl₂ (80.59 mg, 0.110 mmol, 0.20 equiv), K₃PO₄ (280.56 mg, 1.322 mmol, 2.40 equiv) and H₂O (1.00 mL, 55.535 mmol, 100.79 equiv) in dioxane (4 mL) was stirred for 2h at under nitrogen atmosphere. The mixture was allowed to cool down to room temperature. The resulting mixture was diluted with water (10 mL). The resulting mixture was extracted with EtOAc (3×50 mL). The combined organic layers were washed with water (3×20 mL), dried over anhydrous Na₂SO₄, filtered and concentrated under reduced pressure. The residue was purified by reverse flash chromatography (C18 silica gel column, eluting with 10% to 50% MeCN/water (0.1% FA) in 10 min with UV detector (254 nm)). This resulted in 3-(4-cyclopropyl-6-methoxypyrimidin-5-yl)-5H-pyrido[3,2-c]pyridazin-6-one (64 mg, 39.35%) as an off-white solid.

LC-MS (ESI) m/z 296.11 [M+H].

### Step 4: 5-({4-[1-Cyclopropyl-4-(Trifluoromethyl)Imidazol-2-Yl]Phenyl}Methyl)-3-(4-Cyclopropyl-6-Methoxypyrimidin-5-Yl)Pyrido[3,2-c]Pyridazin-6-One

A solution of 3-(4-cyclopropyl-6-methoxypyrimidin-5-yl)-5H-pyri do[3,2-c]pyridazin-6-one (64 mg, 0.217 mmol, 1 equiv), 1,1,3,3-tetramethylguanidine (50 mg, 0.434 mmol, 2.00 equiv) and 2-[4-(chloromethyl)phenyl]-1-cyclopropyl-4-(trifluoromethyl)imidazole (130 mg, 0.432 mmol, 1.99 equiv) in DMF (2 mL) was stirred for 2 h at room temperature under nitrogen atmosphere. The resulting mixture was diluted with water (10 mL). The resulting mixture was extracted with EtOAc (3×50 mL). The combined organic layers were washed with water (3×20 mL), dried over anhydrous Na₂SO₄, filtered and concentrated under reduced pressure. The crude product (29 mg) was purified by Prep-HPLC with the following conditions (Column: XBridge Prep OBD C18 Column 30*150 mm, 51 μm; Mobile Phase A: Water(10 mmol/L NH₄HCO₃+0.1% NH₃.H₂O), Mobile Phase B: ACN; Flow rate: 60 mL/min; Gradient: 32% B to 50% B in 10 min; Wave Length: 254 nm/220 nm; RT1(min): 1) to afford 5-({4-[1-cyclopropyl-4-(trifluoromethyl)imidazol-2-yl]phenyl}methyl)-3-(4-cyclopropyl-6-methoxypyrimidin-5-yl)pyrido[3,2-c]pyridazin-6-one (10.2 mg, 8.21%) as an off-white solid.

LC-MS (ESI) m/z 560 [M+H].

H-NMR (400 MHz, DMSO-d6) δ 8.70 (s, 1H), 8.38 (dd, J=9.8, 0.8 Hz, 1H), 7.98 (d, J=0.9 Hz, 1H), 7.91 (d, J=1.4 Hz, 1H), 7.88-7.84 (m, 2H), 7.41-7.36 (m, 2H), 7.18 (d, J=9.8 Hz, 1H), 5.54 (s, 2H), 3.70 (s, 4H), 1.70 (tt, J=8.2, 4.6 Hz, 1H), 1.02 (dt, J=4.5, 3.0 Hz, 2H), 1.00-0.93 (m, 2H), 0.93-0.87 (m, 2H), 0.81 (dt, J=8.2, 3.3 Hz, 2H).

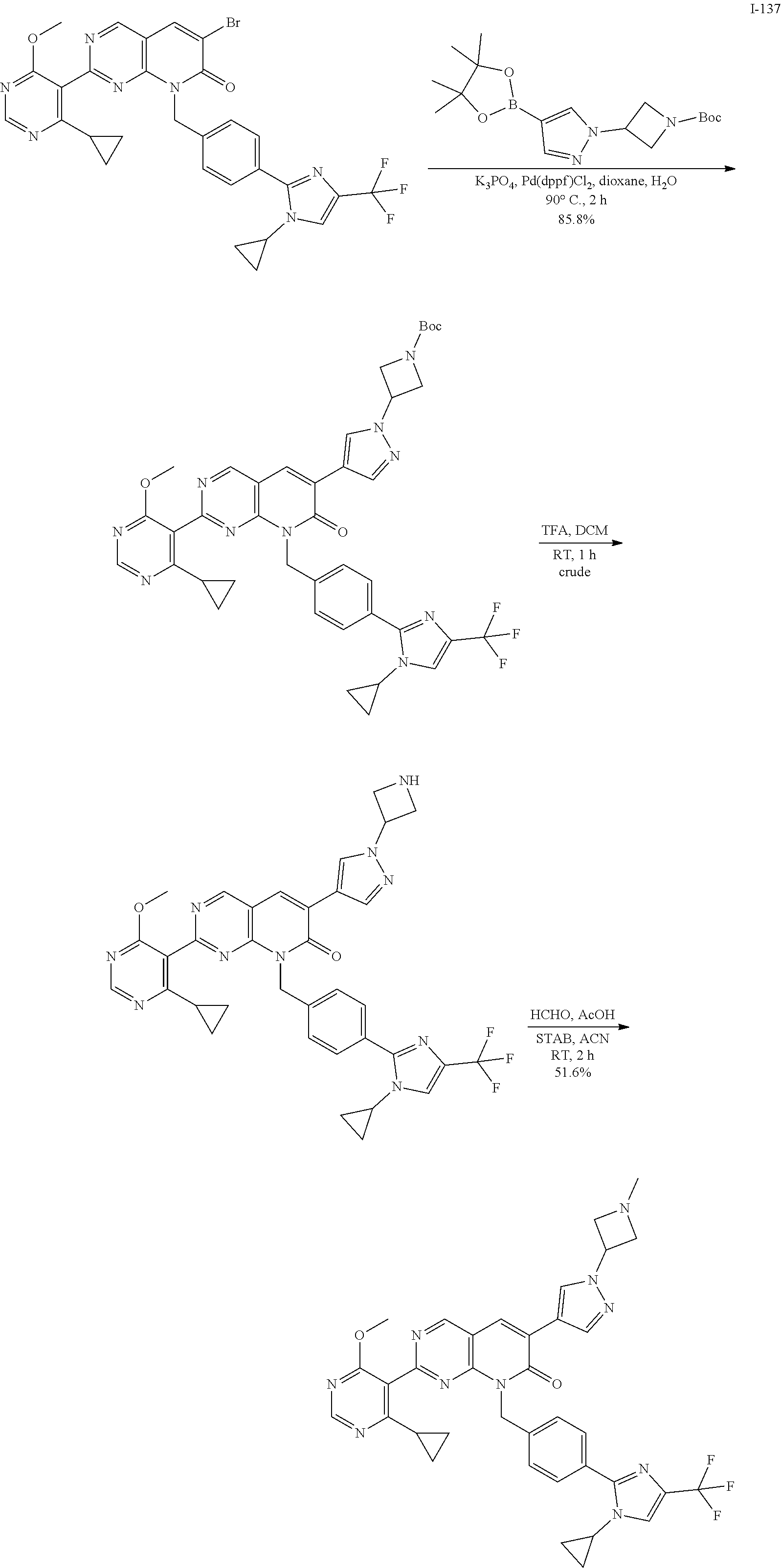 [SMILES: COc1ncnc(C2CC2)c1-c1ncc2cc(Br)c(=O)n(Cc3ccc(-c4nc(C(F)(F)F)cn4C4CC4)cc3)c2n1 ; CC(C)(C)OC(=O)N1CC(n2cc(B3OC(C)(C)C(C)(C)O3)cn2)C1 ; COc1ncnc(C2CC2)c1-c1ncc2cc(-c3cnn(C4CN(C(=O)OC(C)(C)C)C4)c3)c(=O)n(Cc3ccc(-c4nc(C(F)(F)F)cn4C4CC4)cc3)c2n1 ; COc1ncnc(C2CC2)c1-c1ncc2cc(-c3cnn(C4CNC4)c3)c(=O)n(Cc3ccc(-c4nc(C(F)(F)F)cn4C4CC4)cc3)c2n1 ; COc1ncnc(C2CC2)c1-c1ncc2cc(-c3cnn(C4CN(C)C4)c3)c(=O)n(Cc3ccc(-c4nc(C(F)(F)F)cn4C4CC4)cc3)c2n1]

### Step 1: tert-butyl 3-{4-[8-({4-[1-cyclopropyl-4-(trifluoromethyl)imidazol-2-yl]phenyl}methyl)-2-(4-cyclopropyl-6-methoxypyrimidin-5-yl)-7-oxopyrido[2,3-d]pyrimidin-6-yl]pyrazol-1-yl}azetidine-1-carboxylate

To a stirred mixture of 6-bromo-8-(14-(1-cyclopropyl-4-yl)pyrido[2,3-d]pyrimidin-7-one (200 mg, 0.313 mmol, 1 equiv) and tert-butyl 3-[4-(4,4,5,5-tetramethyl-1,3,2-dioxaborolan-2-yl)pyrazol-1-yl]azetidine-1-carboxylate (164.1 mg, 0.470 mmol, 1.5 equiv) in dioxane (2.4 mL) and H₂O (0.6 mL) were added K₃PO₄ (166.2 mg, 0.782 mmol, 2.5 equiv) and Pd(dppf)Cl₂ (45.8 mg, 0.063 mmol, 0.2 equiv) at room temperature under nitrogen atmosphere. The resulting mixture was stirred for 2 h at under nitrogen atmosphere. The mixture was allowed to cool down to room temperature. The resulting mixture was filtered, the filter cake was washed with acetonitrile (3×5 mL). The filtrate was concentrated under reduced pressure. The residue was purified by reverse flash chromatography (C18 silica gel column, eluting with 0% to 100% MeCN/water (0.1% FA) in 10 min with UV detector (254 nm)) to afford tert-butyl 3-{4-[8-({4-[1-cyclopropyl-4-(trifluoromethyl)imidazo]-2-yl]phenyl}methyl)-2-(4-cyclopropyl-6-methoxypyrimidin-5-yl)-7-oxopyrido[2,3-d]pyrimidin-6-yl]pyrazol-1-yl}azetidine-1-carboxylate (210 mg, 85.86%) as a yellow oil.

LC-MS (ESI) m/z 781.3 [M+H]

### Step 2: 6-[1-(Azetidin-3-Yl)Pyrazol-4-Yl]-8-({4-[1-Cyclopropyl-4-(Trifluoromethyl)Imidazol-2-Yl]Phenyl}Methyl)-2-(4-Cyclopropyl-6-Methoxypyrimidin-5-Yl)Pyrido[2,3-d]Pyrimidin-7-One

A mixture of tert-butyl 3-{4-[8-({4-[1-cyclopropyl-4-(trifluoromethyl)imidazo]-2-yl]phenyl}methyl)-2-(4-cyclopropyl-6-methoxypyrimidin-5-yl)-7-oxopyrido[2,3-d]pyrimidin-6-yl]pyrazol-1-yl}azetidine-1-carboxylate (180 mg, 0.231 mmol, 1 equiv) in TFA (1 mL) and DCM (3 mL) was stirred for 1 h at room temperature under nitrogen atmosphere. The residue was basified to pH 8 with saturated NaHCO₃(aq.). The resulting mixture was extracted with EtOAc (3×20 mL). The combined organic layers were washed with brine (3×20 mL), dried over anhydrous Na₂SO₄, filtered and concentrated under reduced pressure to afford 6-[1-(azetidin-3-yl)pyrazol-4-yl]-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (240 mg, crude) as a yellow oil.

LC-MS (ESI) m/z 681.3 [M+H]

### Step 3: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-[1-(1-Methylazetidin-3-Yl)Pyrazol-4-Yl]Pyrido[2,3-d]Pyrimidin-7-One

A mixture of 6-[1-(azetidin-3-yl)pyrazol-4-yl]-8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (80 mg, 0.118 mmol, 1 equiv),HCHO (28.6 mg, 0.354 mmol, 3 equiv, 37%) and AcOH (70.6 mg, 1.180 mmol, 10 equiv) in ACN (1.6 mL) was stirred for at room temperature under nitrogen atmosphere. To the above mixture was added STAB (74.7 mg, 0.354 mmol, 3 equiv) at room temperature. The resulting mixture was stirred for additional 2 h at room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (90 mg) was purified by Prep-HPLC with the following conditions (Column: XBridge Shield RP18 OBD Column 30*150 mm, 51 μm; Mobile Phase A: Water(10 mmol/L NH₄HCO₃+0.1% NH₃.H₂O), Mobile Phase B: ACN; Flow rate: 60 mL/min; Gradient: isocratic 39% B to 55% B in 10 min; Wave Length: 254 nm/220 nm; RT1(min): 7.22) to afford 8-({4-[1-cyclopropyl-4-(trifluoromethyl)imidazo]-2-yl]phenyl methyl)-2-(4-cyclopropyl-6-methoxypyrimidin-5-yl)-6-[1-(1-methylazetidin-3-yl)pyrazol-4-yl]pyrido[2,3-d]pyrimidin-7-one (42.5 mg, 51.69%) as an off-white solid.

LC-MS (ESI) m/z 695.1 [M+H]

¹H NMR (400 MHz, DMSO-d6) δ 9.26 (s, 1H), 8.68 (d, J=9.1 Hz, 2H), 8.55 (s, 1H), 8.24 (s, 1H), 7.91 (s, 1H), 7.82 (d, J=8.0 Hz, 2H), 7.42 (d, J=8.1 Hz, 2H), 5.69 (s, 2H), (p, J=6.7 Hz, 1H), 3.84 (s, 3H), 3.69 (q, J=6.3, 5.5 Hz, 3H), 3.40 (t, J=7.0 Hz, 2H), 2.33 (s, 3H), 1.78 (td, J=7.9, 4.0 Hz, 1H), 1.03 (q, J=3.8 Hz, 2H), 0.95 (dd, J=7.4, 5.1 Hz, 2H), 0.90 (t, J=4.3 Hz, 2H), 0.78 (dd, J=8.0, 3.3 Hz, 2H).

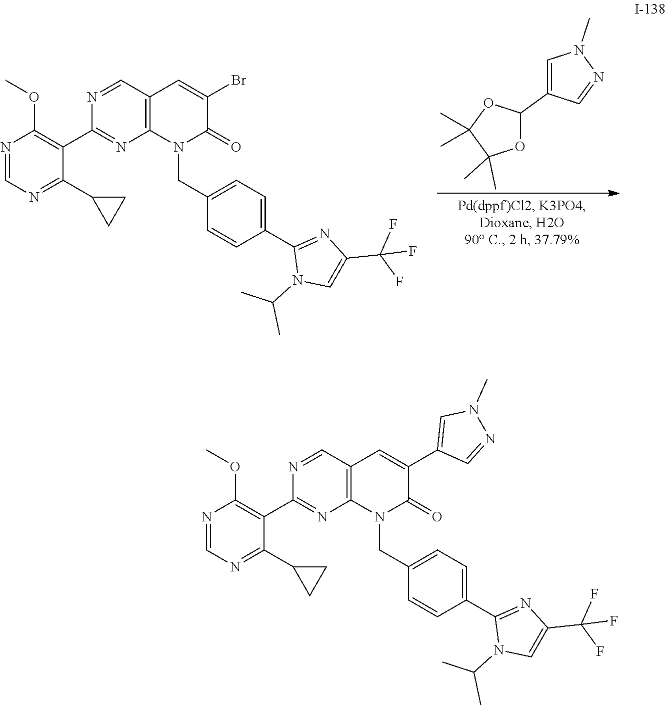 [SMILES: COc1ncnc(C2CC2)c1-c1ncc2cc(Br)c(=O)n(Cc3ccc(-c4nc(C(F)(F)F)cn4C(C)C)cc3)c2n1 ; Cn1cc(C2OC(C)(C)C(C)(C)O2)cn1 ; COc1ncnc(C2CC2)c1-c1ncc2cc(-c3cnn(C)c3)c(=O)n(Cc3ccc(-c4nc(C(F)(F)F)cn4C(C)C)cc3)c2n1]

A solution of 6-bromo-2-(4-cyclopropyl-6-methoxypyrimidin-5-yl)-8-({4-[1-isopropyl-4-(trifluoromethyl)imidazo]-2-yl]phenyl methyl)pyrido[2,3-d]pyrimidin-7-one (50 mg, mmol, 1 equiv), 1-methyl-4-(4,4,5,5-tetramethyl-1,3,2-dioxaborolan-2-yl)pyrazole (20 mg, mmol, 1.23 equiv), Pd(dppf)Cl₂ (11 mg, 0.015 mmol, 0.19 equiv), K₃PO₄ (40 mg, 0.188 mmol, 2.41 equiv) and H₂O (0.5 mL, 27.755 mmol, 355.52 equiv) in dioxane (2 mL) was stirred for 2 h at 90° C. under nitrogen atmosphere. The mixture was allowed to cool down to room temperature. The resulting mixture was extracted with EtOAc (3×50 mL). The combined organic layers were washed with water (3×20 mL), dried over anhydrous Na₂SO₄, filtered and concentrated under reduced pressure. The crude product (50 mg) was purified by Prep-HPLC with the following conditions (Column: XBridge Prep OBD C18 Column 30*150 mm, 51 μm; Mobile Phase A: Water(10 mmol/L NH₄HCO₃+0.1% NH₃.H₂O), Mobile Phase B: ACN; Flow rate: 60 mL/min; Gradient: 40% B to 58% B in 10 min; Wave Length: 254 nm/220 nm; RT1(min): 1) to afford 2-(4-cyclopropyl-6-methoxypyrimidin-5-yl)-8-({4-[1-isopropyl-4-(trifluoromethyl)imidazol-2-yl]phenyl}methyl)-6-(1-methylpyrazol-4-yl)pyrido[2,3-d]pyrimidin-7-one (19.2 mg, 37.79%) as an off-white solid.

LC-MS (ESI) miz 642.25 [M+1-1].

H-NMR (400 MHz, DMSO-d6) δ 9.25 (s, 1H), 8.69 (s, 1H), 8.50 (d, J=2.0 Hz, 2H), 8.15 (s, 2H), 7.47 (q, J=8.3 Hz, 4H), 5.69 (s, 2H), 4.41 (p, J=6.6 Hz, 1H), 3.91 (s, 3H), 3.84 (s, 3H), 1.76 (tt, J=8.1, 4.6 Hz, 1H), 1.37 (d, J=6.6 Hz, 6H), 1.02 (p, J=3.4 Hz, 2H), 0.76 (dq, J=7.0, 3.5 Hz, 2H).

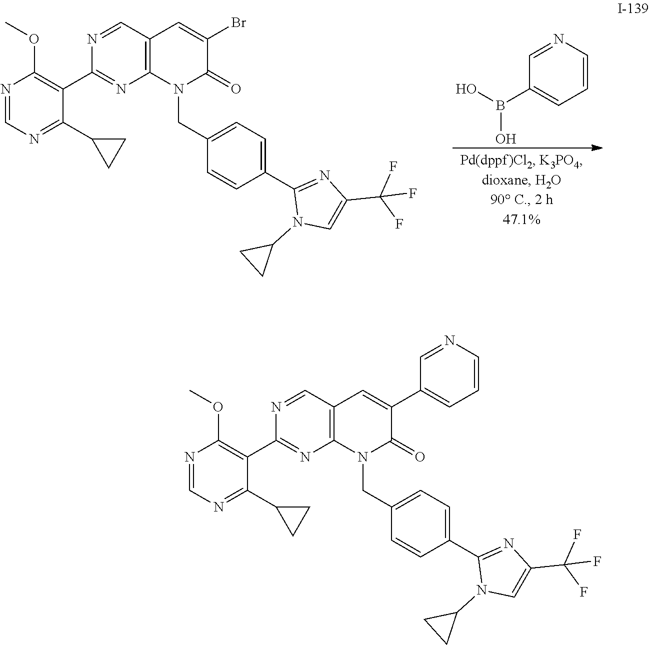 [SMILES: COc1ncnc(C2CC2)c1-c1ncc2cc(Br)c(=O)n(Cc3ccc(-c4nc(C(F)(F)F)cn4C4CC4)cc3)c2n1 ; OB(O)c1cccnc1 ; COc1ncnc(C2CC2)c1-c1ncc2cc(-c3cccnc3)c(=O)n(Cc3ccc(-c4nc(C(F)(F)F)cn4C4CC4)cc3)c2n1]

To a stirred mixture of 6-bromo-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (70 mg, 0.110 mmol, 1 equiv) and pyridin-3-ylboronic acid (20.2 mg, 0.165 mmol, 1.5 equiv) in dioxane (1.2 mL) and H₂O (0.3 mL) were added Pd(dppf)C12 (16.05 mg, 0.022 mmol, 0.2 equiv) and K₃PO₄ (58.2 mg, 0.275 mmol, 2.5 equiv) at room temperature under nitrogen atmosphere. The resulting mixture was stirred for 2 h at under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (80 mg) was purified by Prep-HPLC with the following conditions (Column: XBridge Prep OBD C18 Column 30*150 mm, 5 μm; Mobile Phase A: Water(10 mmol/L NH₄HCO₃), Mobile Phase B: ACN; Flow rate: 60 mL/min; Gradient: 37% B to 83% B in 10 min; Wave Length: 254 nm/220 nm; RT1(min): 2) to afford 8-({4-[1-cyclopropyl-4-(trifluoromethyl)imidazo]-2-yl]phenyl methyl)-2-(4-cyclopropyl-6-methoxypyrimidin-5-yl)-6-(pyridin-3-yl)pyrido[2,3-d]pyrimidin-7-one (33 mg, 47.18%) as an off-white solid.

LC-MS (ESI) m/z 637.2 [M+H]

¹H NMR (400 MHz, DMSO-d6) δ 9.35 (s, 1H), 8.92 (d, J=2.3 Hz, 1H), 8.71 (s, 1H), 8.64 (dd, J=4.8, 1.6 Hz, 1H), 8.48 (s, 1H), 8.17 (dt, J=8.2, 2.1 Hz, 1H), 7.91 (d, J=1.4 Hz, 1H), 7.82 (d, J=8.1 Hz, 2H), 7.53 (dd, J=7.9, 4.8 Hz, 1H), 7.46 (d, J=8.1 Hz, 2H), 5.67 (s, 2H), 3.85 (s, 3H), 3.69 (dq, J=7.6, 3.8 Hz, 1H), 1.77 (tt, J=8.2, 4.8 Hz, 1H), 1.03 (d, J=5.0 Hz, 2H), (dd, J=7.4, 4.9 Hz, 2H), 0.91 (d, J=4.2 Hz, 2H), 0.79 (dd, J=7.9, 3.4 Hz, 2H).

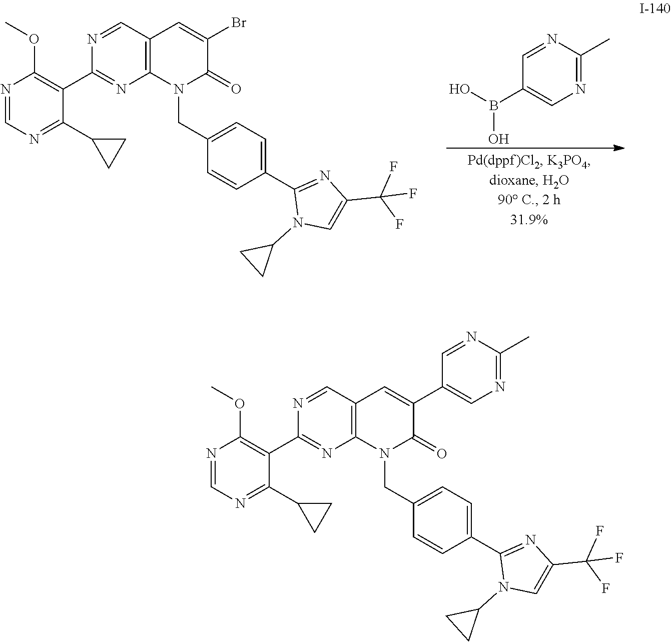

To a stirred mixture of 6-bromo-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (70 mg, 0.110 mmol, 1 equiv) and 2-methylpyrimidin-5-ylboronic acid (22.7 mg, 0.165 mmol, 1.5 equiv) in dioxane (1.2 mL) and H₂O (0.3 mL) were added Pd(dppf)Cl₂ (16.1 mg, 0.022 mmol, 0.2 equiv) and K₃PO₄ (58.2 mg, 0.275 mmol, 2.5 equiv) at room temperature under nitrogen atmosphere. The resulting mixture was stirred for 2 h at under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (80 mg) was purified by Prep-HPLC with the following conditions (Column: XBridge Prep OBD C18 Column 30*150 mm, 5 μm; Mobile Phase A: Water(10 mmol/L NH₄HCO₃), Mobile Phase B: ACN; Flow rate: 60 mL/min; Gradient: 35% B to 51% B in 10 min; Wave Length: 254 nm/220 nm; RT1(min): 10.48) to afford 8-({4-[1-cyclopropyl-4-(trifluoromethyl)imidazol-2-yl]phenyl methyl)-2-(4-cyclopropyl-6-methoxypyrimidin-5-yl)-6-(2-methylpyrimidin-5-yl)pyrido[2,3-d]pyrimidin-7-one (23.1 mg, 31.94%) as an off-white solid.

LC-MS (ESI) m/z 652.2 [M+H]

¹H NMR (400 MHz, DMSO-d6) δ 9.35 (s, 1H), 9.07 (s, 2H), 8.71 (s, 1H), 8.57 (s, 1H), 7.91 (s, 1H), 7.82 (d, J=8.1 Hz, 2H), 7.46 (d, J=8.0 Hz, 2H), 5.67 (s, 2H), 3.85 (s, 3H), 3.72-3.67 (m, 1H), 2.70 (s, 3H), 1.76 (t, J=5.7 Hz, 1H), 1.06-1.00 (m, 2H), 1.00-0.93 (m, 2H), 0.90 (t, J=4.3 Hz, 2H), 0.79 (dd, J=7.9, 3.5 Hz, 2H).

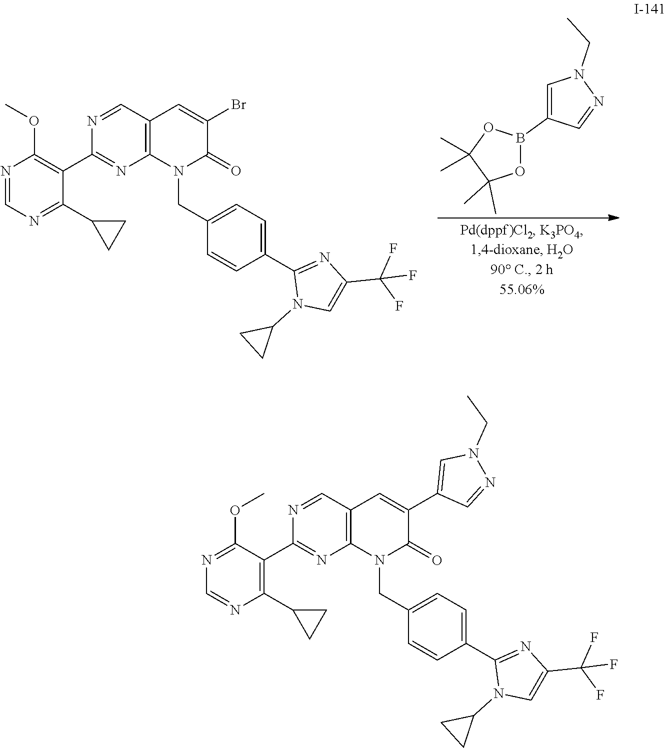 [SMILES: COc1ncnc(C2CC2)c1-c1ncc2cc(Br)c(=O)n(Cc3ccc(-c4nc(C(F)(F)F)cn4C4CC4)cc3)c2n1 ; CCn1cc(B2OC(C)(C)C(C)(C)O2)cn1 ; CCn1cc(-c2cc3cnc(-c4c(OC)ncnc4C4CC4)nc3n(Cc3ccc(-c4nc(C(F)(F)F)cn4C4CC4)cc3)c2=O)cn1]

To a stirred solution of 6-bromo-8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (70 mg, 0.110 mmol, 1 equiv) and 1-ethyl-4-(4,4,5,5-tetramethyl-1,3,2-dioxaborolan-2-yl)pyrazole (36.5 mg, 0.165 mmol, 1.5 equiv) in 1,4-dioxane (0.8 mL) and H₂O (0.2 mL) were added pd(dppf)Cl₂ (8.0 mg, 0.011 mmol, 0.1 equiv) and K₃PO₄ (69.8 mg, 0.330 mmol, 3.0 equiv) at room temperature under nitrogen atmosphere. The resulting mixture was stirred for 2 h at 90° C. under nitrogen atmosphere. The mixture was allowed to cool down to room temperature. The resulting mixture was diluted with water (20 mL). The resulting mixture was extracted with EtOAc (3×50 mL). The combined organic layers were washed with brine (2×50 mL), dried over anhydrous Na₂SO₄, filtered and concentrated under reduced pressure. The crude product (70 mg) was purified by Prep-HPLC with the following conditions (Column: Sunfire prep C18 column, 30*150 mm, 5 μm; Mobile Phase A: Water(0.05% TFA), Mobile Phase B: ACN; Flow rate: 60 mL/min; Gradient: 48% B to 65% B in 10 min; Wave Length: 254 nm/220 nm; RT1(min): 10.32) to afford 8-({4-[1-cyclopropyl-4-(trifluoromethyl)imidazo]-2-yl]phenyl}methyl)-2-(4-cyclopropyl-6-methoxypyrimidin-5-yl)-6-(1-ethylpyrazol-4-yl)pyrido[2,3-d]pyrimidin-7-one (39.5 mg, 55.06%, purity:99.9%) as an off-white solid.

LC-MS (ESI) m/z 654.25 [M+H]

¹H NMR (400 MHz, DMSO-d6) δ 9.25 (s, 1H), 8.69 (s, 1H), 8.52 (d, J=13.0 Hz, 2H), 8.17 (s, 1H), 7.91 (d, J=1.4 Hz, 1H), 7.85-7.78 (m, 2H), 7.42 (d, J=8.2 Hz, 2H), 5.68 (s, 2H), 4.21 (d, J=7.3 Hz, 2H), 3.84 (s, 3H), 3.68 (tt, J=7.4, 4.0 Hz, 1H), 1.78 (tt, J=8.2, 4.6 Hz, 1H), 1.40 (t, J=7.2 Hz, 3H), 1.02 (q, J=3.8 Hz, 2H), 1.00-0.92 (m, 2H), 0.89 (tt, J=6.8, 3.4 Hz, 2H), 0.82-0.73 (m, 2H).

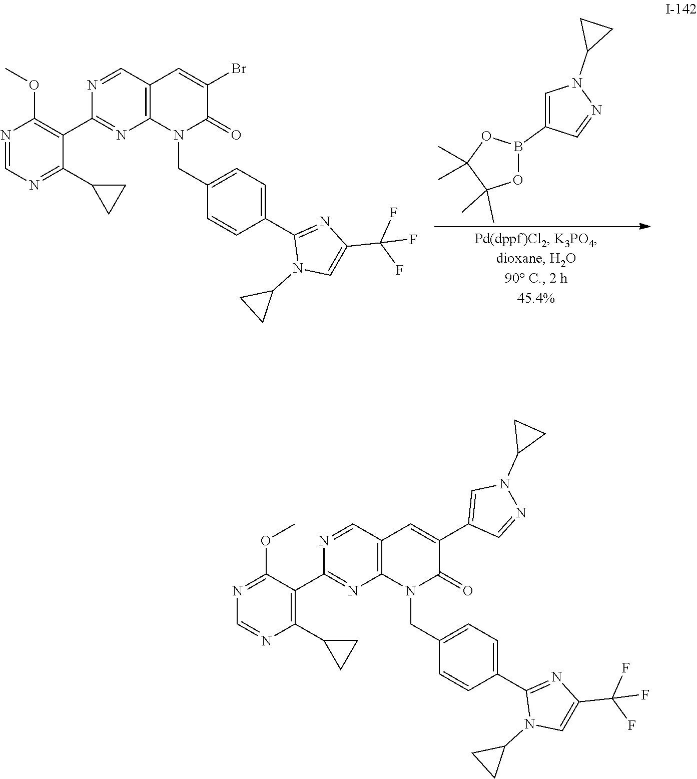 [SMILES: COc1ncnc(C2CC2)c1-c1ncc2cc(Br)c(=O)n(Cc3ccc(-c4nc(C(F)(F)F)cn4C4CC4)cc3)c2n1 ; CC1(C)OB(c2cnn(C3CC3)c2)OC1(C)C ; COc1ncnc(C2CC2)c1-c1ncc2cc(-c3cnn(C4CC4)c3)c(=O)n(Cc3ccc(-c4nc(C(F)(F)F)cn4C4CC4)cc3)c2n1]

To a stirred mixture of 6-bromo-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (70 mg, 0.110 mmol, 1 equiv) and 1-cyclopropyl-4-(4,4,5,5-tetramethyl-1,3,2-dioxaborolan-2-yl)pyrazole (38.5 mg, 0.165 mmol, 1.5 equiv) in dioxane (1.2 mL) and H₂O (0.3 mL) were added Pd(dppf)Cl₂ (16.1 mg, 0.022 mmol, 0.2 equiv) and K₃PO₄ (58.2 mg, 0.275 mmol, 2.5 equiv) at room temperature under nitrogen atmosphere. The resulting mixture was stirred for 2 h at 90° C. under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (80 mg) was purified by Prep-HPLC with the following conditions (Column: XBridge Shield RP18 OBD Column 30*150 mm, 51 μm; Mobile Phase A: Water(10 mmol/L NH₄HCO₃), Mobile Phase B: ACN; Flow rate: 60 mL/min; Gradient: 38% B to 58% B in 10 min; Wave Length: 254 nm/220 nm; RT1(min): 11.27) to afford 8-({4-[1-cyclopropyl-4-(trifluoromethyl)imidazo]-2-yl]phenyl}methyl)-2-(4-cyclopropyl-6-methoxypyrimidin-5-yl)-6-(1-cyclopropylpyrazol-4-yl)pyrido[2,3-d]pyrimidin-7-one (33.4 mg, 45.44%) as an off-white solid.

LC-MS (ESI) m/z 666.2 [M+H]

¹H NMR (400 MHz, DMSO-d6) δ 9.24 (s, 1H), 8.69 (s, 1H), 8.53 (d, J=15.0 Hz, 2H), 8.16 (s, 1H), 7.91 (d, J=1.4 Hz, 1H), 7.85-7.78 (m, 2H), 7.41 (d, J=8.2 Hz, 2H), 5.68 (s, 2H), 3.88-3.78 (m, 4H), 3.69 (tt, J=7.3, 4.0 Hz, 1H), 1.78 (tt, J=8.1, 4.6 Hz, 1H), 1.09 (hept, J=3.6 Hz, 2H), 1.01 (dt, J=6.9, 3.3 Hz, 4H), 0.95 (dd, J=7.5, 5.0 Hz, 2H), 0.91 (d, J=6.3 Hz, 2H), 0.77 (dd, J=7.8, 3.5 Hz, 2H).

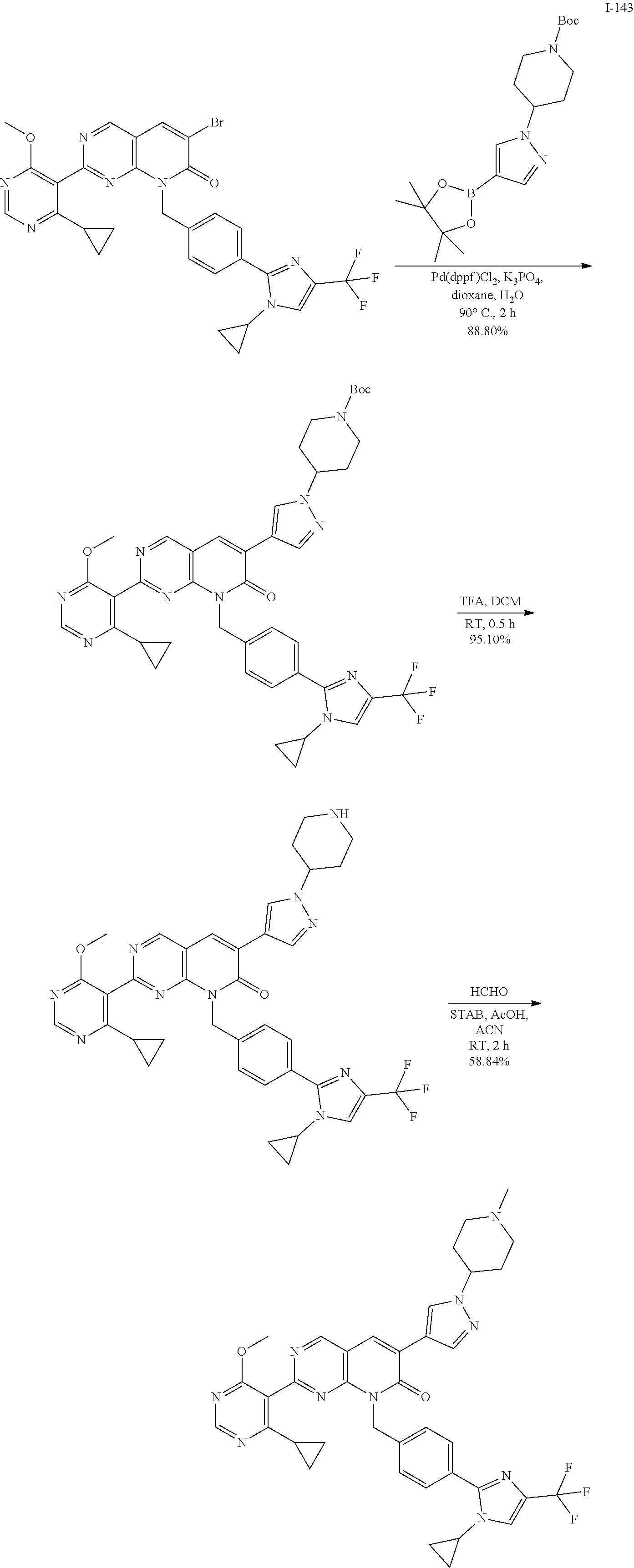 [SMILES: COc1ncnc(C2CC2)c1-c1ncc2cc(Br)c(=O)n(Cc3ccc(-c4nc(C(F)(F)F)cn4C4CC4)cc3)c2n1 ; CC(C)(C)OC(=O)N1CCC(n2cc(B3OC(C)(C)C(C)(C)O3)cn2)CC1 ; COc1ncnc(C2CC2)c1-c1ncc2cc(-c3cnn(C4CCN(C(=O)OC(C)(C)C)CC4)c3)c(=O)n(Cc3ccc(-c4nc(C(F)(F)F)cn4C4CC4)cc3)c2n1 ; COc1ncnc(C2CC2)c1-c1ncc2cc(-c3cnn(C4CCNCC4)c3)c(=O)n(Cc3ccc(-c4nc(C(F)(F)F)cn4C4CC4)cc3)c2n1 ; "HCHO" ; COc1ncnc(C2CC2)c1-c1ncc2cc(-c3cnn(C4CCN(C)CC4)c3)c(=O)n(Cc3ccc(-c4nc(C(F)(F)F)cn4C4CC4)cc3)c2n1]

### Step 1: Tert-Butyl 4-{4-[8-({4-[1-Cyclopropyl-4-(Trifluoromethyl)Imidazol-2-Yl]Phenyl}Methyl)-2-(4-Cyclopropyl-6-Methoxypyrimidin-5-Yl)-7-Oxopyrido[2,3-d]Pyrimidin-6-Yl]Pyrazol-1-Yl}Piperidine-1-Carboxylate

To a stirred solution of 6-bromo-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (80 mg, 0.125 mmol, 1 equiv) and tert-butyl 4-[4-(4,4,5,5-tetramethyl-1,3,2-dioxaborolan-2-yl)pyrazol-1-yl]piperidine-1-carboxylate (56.7 mg, 0.150 mmol, 1.20 equiv) in dioxane (2 mL) and H₂O (0.4 mL) were added K₃PO₄ (66.8 mg, 0.315 mmol, 2.51 equiv) and Pd(dppf)C12 (9.2 mg, 0.013 mmol, 0.10 equiv) at room temperature. The resulting mixture was stirred for 2 h at 90° C. under nitrogen atmosphere. The mixture was purified by reversed-phase flash chromatography (C18 silica gel column, eluting with 60% to 80% MeCN/water (0.1% FA) in 10 min with UV detector (254 nm)) to afford tert-butyl 4-{4-[8-({4-[1-cyclopropyl-4-(trifluoromethyl)imidazo]-2-yl]phenyl}methyl)-2-(4-cyclopropyl-6-methoxypyrimidin-5-yl)-7-oxopyrido[2,3-d]pyrimidin-6-yl]pyrazol-1-yl}piperidine-1-carboxylate (90 mg, 88.80%) as a light yellow solid.

LC-MS (ESI) m/z 809.3 [M+H]

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-[1-(Piperidin-4-Yl)Pyrazol-4-Yl]Pyrido[2,3-d]Pyrimidin-7-One

To a stirred solution of tert-butyl 4-{4-[8-({4-[1-cyclopropyl-4-(trifluoromethyl)imidazo]-2-yl]phenyl methyl)-2-(4-cyclopropyl-6-methoxypyrimidin-5-yl)-7-oxopyrido[2,3-d]pyrimidin-6-yl]pyrazol-1-yl}piperidine-1-carboxylate (90 mg, 0.111 mmol, 1 equiv) in DCM (3 mL) was added TFA (1 mL) at room temperature. The resulting mixture was stirred for 30 min at room temperature. The resulting mixture was concentrated under reduced pressure. The residue was purified by reversed-phase flash chromatography (C18 silica gel column, eluting with 50% to 80% MeCN/water (0.1% FA) in 10 min with UV detector (254 nm)) to afford 8-({4-[1-cyclopropyl-4-(trifluoromethyl)imidazol-2-yl]phenyl methyl)-2-(4-cyclopropyl-6-methoxypyrimidin-5-yl)-6-[1-(piperidin-4-yl)pyrazol-4-yl]pyrido[2,3-d]pyrimidin-7-one (75 mg, 95.10%) as a light yellow solid.

LC-MS (ESI) m/z 709.2 [M+H]

### Step 3: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-[1-(1-Methylpiperidin-4-Yl)Pyrazol-4-Yl]Pyrido[2,3-d]Pyrimidin-7-One

To a stirred solution of 8-({4-[1-cyclopropyl-4-(trifluoromethyl)imidazol-2-yl]phenyl methyl)-2-(4-cyclopropyl-6-methoxypyrimidin-5-yl)-6-[1-(piperidin-4-yl)pyrazol-4-yl]pyrido[2,3-d]pyrimidin-7-one (30 mg, 0.042 mmol, 1 equiv) and HCHO (4 mg, 0.133 mmol, 3.15 equiv) in ACN (2 mL) were added AcOH (25.4 mg, 0.423 mmol, 9.99 equiv) at room temperature under nitrogen atmosphere. The resulting mixture was stirred for 5 min at room temperature under nitrogen atmosphere. To the above mixture was added STAB (26.9 mg, 0.127 mmol, 3.00 equiv) at room temperature. The resulting mixture was stirred for additional 2 h at room temperature. The resulting mixture was diluted with water (0.3 mL). The mixture was purified by Prep-HPLC with the following conditions (Column: XBridge Prep Phenyl OBD Column 19*250 mm, 5 μm; Mobile Phase A: Water(10 mmol/L NH₄HCO₃+0.1% NH₃.H₂O), Mobile Phase B: ACN; Flow rate: 60 mL/min; Gradient: 39% B to 55% B in 10 min; Wave Length: 254 nm/220 nm; RT1(min): 8.79) to afford 8-({4-[1-cyclopropyl-4-(trifluoromethyl)imidazo]-2-yl]phenyl methyl)-2-(4-cyclopropyl-6-methoxypyrimidin-5-yl)-6-[1-(1-methylpiperidin-4-yl)pyrazol-4-yl]pyrido[2,3-d]pyrimidin-7-one (18 mg, 58.84%) as an off-white solid.

LC-MS (ESI) m/z 723.4 [M+H]

¹H NMR (400 MHz, DMSO-d6) δ 9.25 (s, 1H), 8.69 (s, 1H), 8.53 (d, J=14.6 Hz, 2H), 8.19 (s, 1H), 7.90 (s, 1H), 7.81 (d, J=8.1 Hz, 2H), 7.41 (d, J=8.1 Hz, 2H), 5.68 (s, 2H), 4.20 (tt, J=9.7, 5.3 Hz, 1H), 3.83 (s, 3H), 3.68 (q, J=6.1, 4.8 Hz, 1H), 2.86 (d, J=10.4 Hz, 2H), 2.21 (s, 3H), 2.10-1.93 (m, 6H), 1.76 (dt, J=8.1, 5.1 Hz, 1H), 1.24 (s, OH), 1.01 (q, J=7.7, 5.6 Hz, 2H), 0.98-0.93 (m, 2H), 0.92-0.86 (m, 2H), 0.77 (dd, J=7.9, 3.4 Hz, 2H).

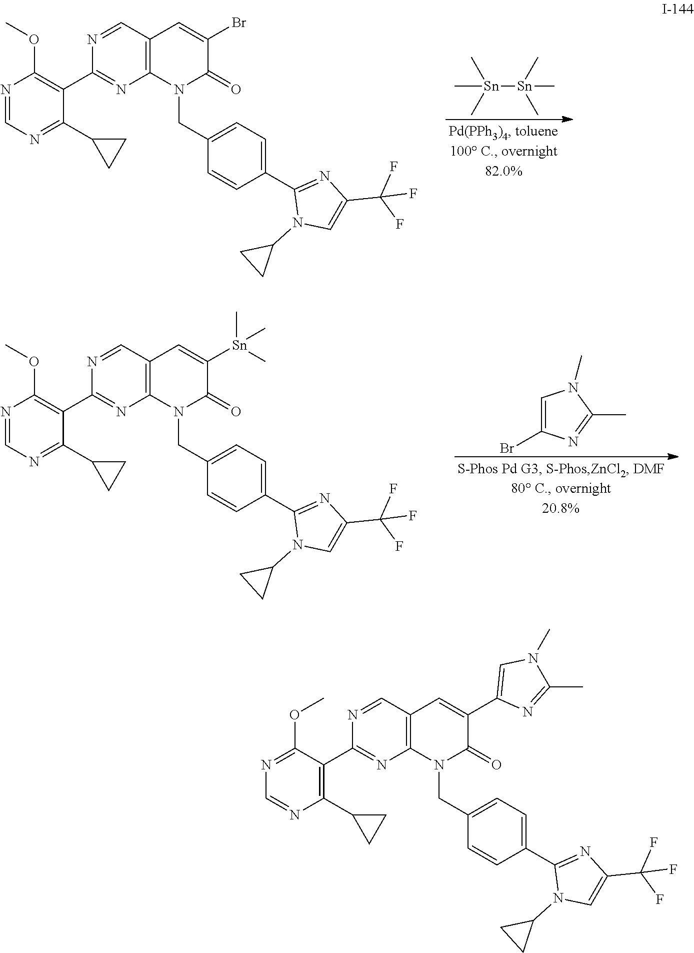

### Step 1: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(Trimethylstannyl)Pyrido[2,3-d]Pyrimidin-7-One

To a stirred mixture of 6-bromo-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (700 mg, 1.096 mmol, 1 equiv) and hexamethyldistannane (718.4 mg, 2.192 mmol, 2 equiv) in toluene (15 mL) was added Pd(PPh₃) 4 (63.3 mg, 0.055 mmol, equiv) at room temperature under nitrogen atmosphere. The resulting mixture was stirred for overnight at 100° C. under nitrogen atmosphere. The mixture was allowed to cool down to room temperature. The resulting mixture was diluted with water (200 mL). The resulting mixture was extracted with EtOAc (3×200 mL). The combined organic layers were washed with brine (3×100 mL), dried over anhydrous Na₂SO₄, filtered and concentrated under reduced pressure. The residue was purified by reverse flash chromatography (C18 silica gel column, eluting with 0% to 100% MeCN/water (0.1% FA) in 10 min with UV detector (254 nm)) to afford 8-({4-[1-cyclopropyl-4-(trifluoromethyl)imidazo]-2-yl]phenyl}methyl)-2-(4-cyclopropyl-6-methoxypyrimidin-5-yl)-6-(trimethylstannyl)pyrido[2,3-d]pyrimidin-7-one (650 mg, 82.0%) as an off-white solid.

LC-MS: MS (ESI) m/z 724.0 [M+H]

¹H NMR (400 MHz, DMSO-d6) δ 9.25 (s, 1H), 8.68 (s, 1H), 8.26 (s, 1H), 7.91 (s, 1H), 7.81 (d, J=8.2 Hz, 2H), 7.38 (d, J=8.1 Hz, 2H), 5.56 (s, 2H), 3.83 (s, 3H), 3.70 (t, J=5.4 Hz, 1H), 1.75-1.68 (m, 1H), 1.01 (s, 2H), 0.99-0.92 (m, 2H), 0.92-0.86 (m, 2H), 0.77 (dd, J=7.9, 3.4 Hz, 2H), 0.32 (t, 9H).

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(1,2-Dimethylimidazol-4-Yl)Pyrido[2,3-d]Pyrimidin-7-One

To a stirred mixture of 8-({4-[1-cyclopropyl-4-(trifluoromethyl)imidazo]-2-yl]phenyl}methyl)-2-(4-cyclopropyl-6-methoxypyrimidin-5-yl)-6-(trimethylstannyl)pyrido[2,3-d]pyrimidin-7-one (80 mg, 0.111 mmol, 1 equiv) and 4-bromo-1,2-dimethylimidazole (38.8 mg, mmol, 2 equiv) in DMF (1.7 mL) were added S-phos (8 mg, 0.019 mmol, 0.18 equiv),S-Phos Pd Gen.3 (8 mg) and ZnCl₂ (15.1 mg, 0.111 mmol, 1 equiv) at room temperature under nitrogen atmosphere. The resulting mixture was stirred for overnight at under nitrogen atmosphere. The mixture was allowed to cool down to room temperature. The resulting mixture was diluted with water (10 mL). The resulting mixture was extracted with EtOAc (3×20 mL). The combined organic layers were washed with brine (3×10 mL), dried over anhydrous Na₂SO₄, filtered and concentrated under reduced pressure. The crude product (90 mg) was purified by Prep-HPLC with the following conditions (Column: Xselect CSH C18 OBD Column 30*150 mm 51 μm; Mobile Phase A: Water(0.1% FA), Mobile Phase B: ACN; Flow rate: 60 mL/min; Gradient: 39% B to 57% B in 8 min; Wave Length: 254 nm/220 nm nm; RT1(min): 2.9) to afford 8-({4-[1-cyclopropyl-4-(trifluoromethyl)imidazo]-2-yl]phenyl}methyl)-2-(4-cyclopropyl-6-methoxypyrimidin-5-yl)-6-(1,2-dimethylimidazol-4-yl)pyrido[2,3-d]pyrimidin-7-one (15.3 mg, 20.8%) as an off-white solid.

LC-MS (ESI) m/z 654.2 [M+H]

¹H NMR (400 MHz, DMSO-d6) δ 9.41 (d, J=3.9 Hz, 1H), 8.68 (dd, J=13.3, 3.8 Hz, 2H), 7.99-7.86 (m, 2H), 7.82 (dd, J=8.2, 3.6 Hz, 2H), 7.46-7.39 (m, 2H), 5.69 (s, 2H), 3.85 (d, J=3.7 Hz, 3H), 3.71-3.66 (m, 1H), 3.63 (d, J=3.8 Hz, 3H), 2.38 (d, J=3.8 Hz, 3H), 1.81-1.74 (m, 1H), 1.05-0.99 (m, 2H), 0.95 (dd, J=7.4, 4.8 Hz, 2H), 0.90 (s, 2H), 0.79 (dt, J=7.8, 3.4 Hz, 2H).

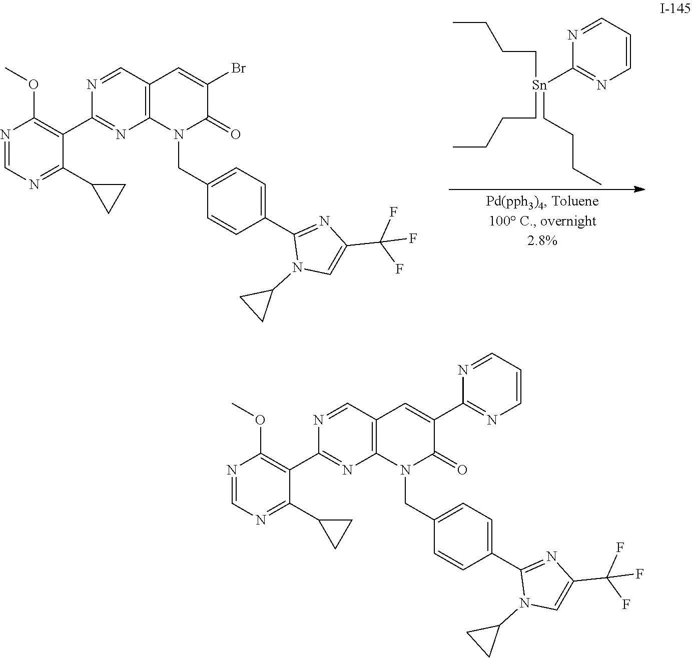 [SMILES: COc1ncnc(C2CC2)c1-c1ncc2cc(Br)c(=O)n(Cc3ccc(-c4nc(C(F)(F)F)cn4C4CC4)cc3)c2n1 ; CCC[CH2][Sn]([CH2]CCC)([CH2]CCC)[c]1ncccn1 ; COc1ncnc(C2CC2)c1-c1ncc2cc(-c3ncccn3)c(=O)n(Cc3ccc(-c4nc(C(F)(F)F)cn4C4CC4)cc3)c2n1]

To a stirred solution of 6-bromo-8-({4-(1-cyclopropyl-4-yl)pyrido[2,3-d]pyrimidin-7-one (100 mg, 0.157 mmol, 1 equiv) and 2-(tributylstannyl)pyrimidine (115.6 mg, 0.314 mmol, 2 equiv) in Toluene (2 mL) was added Pd(PPh₃) 4 (9.0 mg, 0.008 mmol, 0.05 equiv) at room temperature under nitrogen atmosphere. The resulting mixture was stirred for overnight at 100° C. under nitrogen atmosphere. The reaction was quenched by the addition of Water (10 mL) at 0° C. The resulting mixture was extracted with EtOAc (3×50 mL). The combined organic layers were washed with brine (2×20 mL), dried over anhydrous Na₂SO₄, filtered and concentrated under reduced pressure. The crude product (30 mg) was purified by Prep-HPLC with the following conditions (Column: Sunfire prep C18 column, 30*150 mm, 51 μm; Mobile Phase A: Water(0.05% TFA), Mobile Phase B: ACN; Flow rate: 60 mL/min; Gradient: 50% B to 70% B in min; Wave Length: 254 nm/220 nm; RT1(min): 10.32) to afford 8-({4-[1-cyclopropyl-4-(trifluoromethyl)imidazo]-2-yl]phenyl}methyl)-2-(4-cyclopropyl-6-methoxypyrimidin-5-yl)-6-(pyrimidin-2-yl)pyrido[2,3-d]pyrimidin-7-one (2.8 mg, 2.80%, purity:99.7%) as an off-white solid.

LC-MS (ESI) m/z 638.20 [M+H]

¹H NMR (400 MHz, DMSO-d6) δ 9.42 (s, 1H), 8.97 (d, J=4.9 Hz, 2H), 8.70 (s, 1H), 8.62 (s, 1H), 7.91 (q, J=1.3 Hz, 1H), 7.87-7.80 (m, 2H), 7.57 (t, J=4.9 Hz, 1H), 7.49-7.42 (m, 2H), 5.65 (s, 2H), 3.86 (s, 3H), 3.70 (tt, J=7.2, 3.9 Hz, 1H), 1.81 (tt, J=8.1, 4.5 Hz, 1H), 1.04 (dq, J=6.3, 3.5 Hz, 2H), 0.98 (td, J=6.8, 6.2, 2.4 Hz, 2H), 0.95-0.84 (m, 2H), 0.80 (dt, J=8.2, 3.4 Hz, 2H).

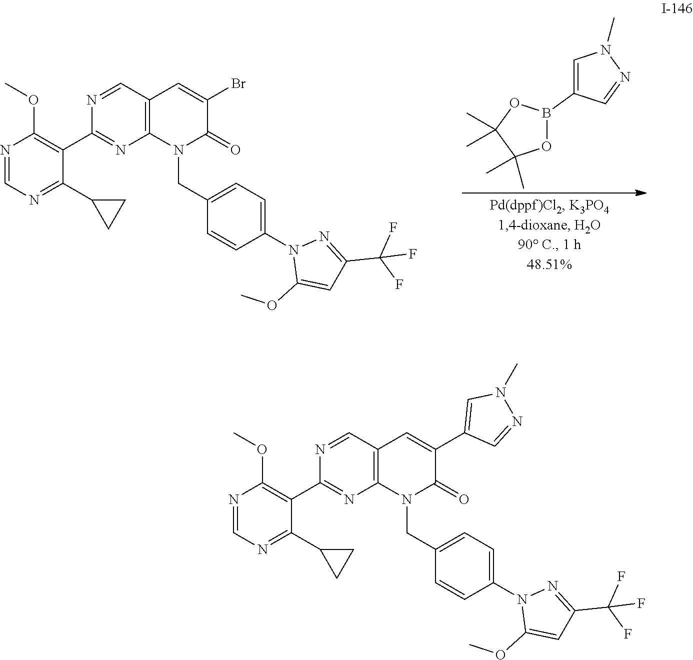 [SMILES: COc1ncnc(C2CC2)c1-c1ncc2cc(Br)c(=O)n(Cc3ccc(-n4nc(C(F)(F)F)cc4OC)cc3)c2n1 ; Cn1cc(B2OC(C)(C)C(C)(C)O2)cn1 ; COc1ncnc(C2CC2)c1-c1ncc2cc(-c3cnn(C)c3)c(=O)n(Cc3ccc(-n4nc(C(F)(F)F)cc4OC)cc3)c2n1]

To a stirred solution of 6-bromo-2-(4-cyclopropyl-6-methoxypyrimidin-5-yl)-8-({4-[5-methoxy-3-(trifluoromethyl)pyrazol-1-yl]phenyl methyl)pyri do[2,3-d]pyrimidin-7-one (80 mg, 0.127 mmol, 1 equiv) and 1-methyl-4-(4,4,5,5-tetramethyl-1,3,2-dioxaborolan-2-yl)pyrazole (39.7 mg, 0.191 mmol, 1.5 equiv) in 1,4-dioxane (0.8 mL) and H₂O (0.2 mL) were added Pd(dppf)C12 (9.3 mg, 0.013 mmol, 0.1 equiv) and K₃PO₄ (81.0 mg, 0.381 mmol, 3.0 equiv) at room temperature under nitrogen atmosphere. The resulting mixture was stirred for 2 h at room temperature under nitrogen atmosphere. The mixture was allowed to cool down to room temperature. The resulting mixture was diluted with water (50 mL). The resulting mixture was extracted with EtOAc (3×50 mL). The combined organic layers were washed with brine (2×50 mL), dried over anhydrous Na₂SO₄, filtered and The crude product (80 mg) was purified by Prep-HPLC with the following conditions (Column: XBridge Prep Phenyl OBD Column 19*250 mm, 51 μm; Mobile Phase A: Water(0.05% TFA), Mobile Phase B: ACN; Flow rate: 60 mL/min; Gradient: 34% B to 52% B in 10 min; Wave Length: 254 nm/220 nm; RT1(min): 13.94) to afford 2-(4-cyclopropyl-6-methoxypyrimidin-5-yl)-8-({4-[5-m ethoxy-3-(trifluoromethyl)pyrazol-1-yl]phenyl methyl)-6-(1-methylpyrazol-4-yl)pyrido[2,3-d]pyrimidin-7-one (39 mg, 48.51%, purity:99.7%) as an off-white solid.

LC-MS (ESI) m/z 630.20 [M+H]

¹H NMR (400 MHz, DMSO-d6) δ 9.24 (s, 1H), 8.69 (s, 1H), 8.49 (d, J=4.4 Hz, 2H), 8.15 (d, J=0.7 Hz, 1H), 7.58-7.50 (m, 2H), 7.47-7.41 (m, 2H), 6.44 (s, 1H), 5.66 (s, 2H), 3.95 (s, 3H), 3.91 (s, 3H), 3.83 (s, 3H), 1.73 (dt, J=8.2, 4.6 Hz, 1H), 1.06-0.99 (m, 2H), 0.78 (dt, J=8.2, 3.3 Hz, 2H).

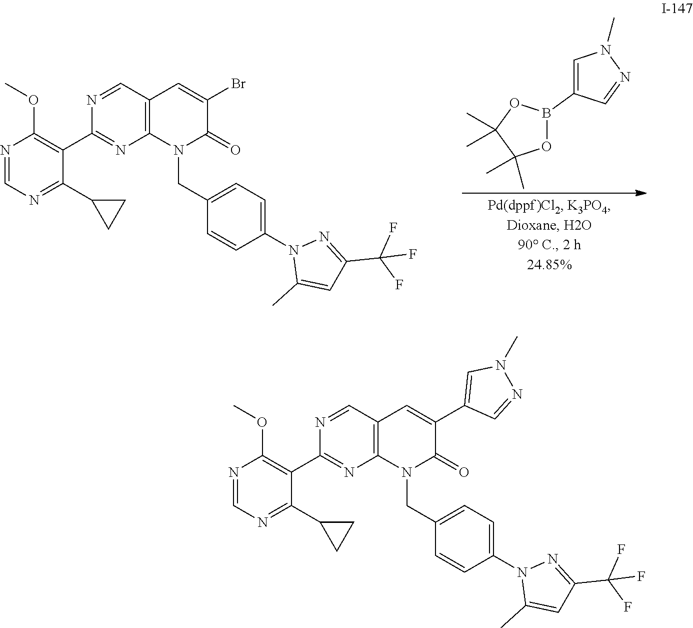 [SMILES: COc1ncnc(C2CC2)c1-c1ncc2cc(Br)c(=O)n(Cc3ccc(-n4nc(C(F)(F)F)cc4C)cc3)c2n1 ; Cn1cc(B2OC(C)(C)C(C)(C)O2)cn1 ; COc1ncnc(C2CC2)c1-c1ncc2cc(-c3cnn(C)c3)c(=O)n(Cc3ccc(-n4nc(C(F)(F)F)cc4C)cc3)c2n1]

A solution of 6-bromo-2-(4-cyclopropyl-6-methoxypyrimidin-5-yl)-8-({4-[5-methyl-3-(trifluoromethyl)pyrazol-1-yl]phenyl 1methyl)pyri do[2,3-d]pyrimidin-7-one (200 mg, mmol, 1 equiv), 1-methyl-4-(4,4,5,5-tetramethyl-1,3,2-dioxaborolan-2-yl)pyrazol e (80 mg, mmol, 1.18 equiv), Pd(dppf)Cl₂ (40 mg, 0.055 mmol, 0.17 equiv), K₃PO₄ (170 mg, 0.801 mmol, 2.45 equiv) and H₂O (2 mL, 111.019 mmol, 339.95 equiv) in dioxane (8 mL) was stirred for 2 h at 90° C. under nitrogen atmosphere. The mixture was allowed to cool down to room temperature. The resulting mixture was extracted with EtOAc (3×20 mL). The combined organic layers were washed with water (3×20 mL), dried over anhydrous Na₂SO₄, filtered and concentrated under reduced pressure. The crude product (200 mg) was purified by Prep-HPLC with the following conditions (Column: XBridge Prep OBD C18 Column 30*150 mm, 51 μm; Mobile Phase A: Water(10 mmol/L NH₄HCO₃), Mobile Phase B: ACN; Flow rate: 60 mL/min; Gradient: 45% B to 60% B in 10 min; Wave Length: 254 nm/220 nm; RT1(min): 10.83) to afford 2-(4-cyclopropyl-6-methoxypyrimidin-5-yl)-8-({4-[5-methyl-3-(trifluoromethyl)pyrazol-1-yl]phenyl}methyl)-6-(1-methylpyrazol-4-yl)pyrido[2,3-d]pyrimidin-7-one (50.6 mg, 24.85%) as an off-white solid.

LC-MS (ESI) m/z 614.5 [M+1-1].

H-NMR-¹H NMR (400 MHz, DMSO-d6) δ 9.25 (s, 1H), 8.69 (s, 1H), 8.50 (d, J=1.9 Hz, 2H), 8.15 (s, 1H), 7.49 (s, 4H), 6.73 (s, 1H), 5.69 (s, 2H), 3.88 (d, J=26.3 Hz, 6H), 2.30 (s, 3H), 1.77 (s, 1H), 1.24 (s, 1H), 1.03 (s, 2H), 0.79 (t, J=4.7 Hz, 2H).

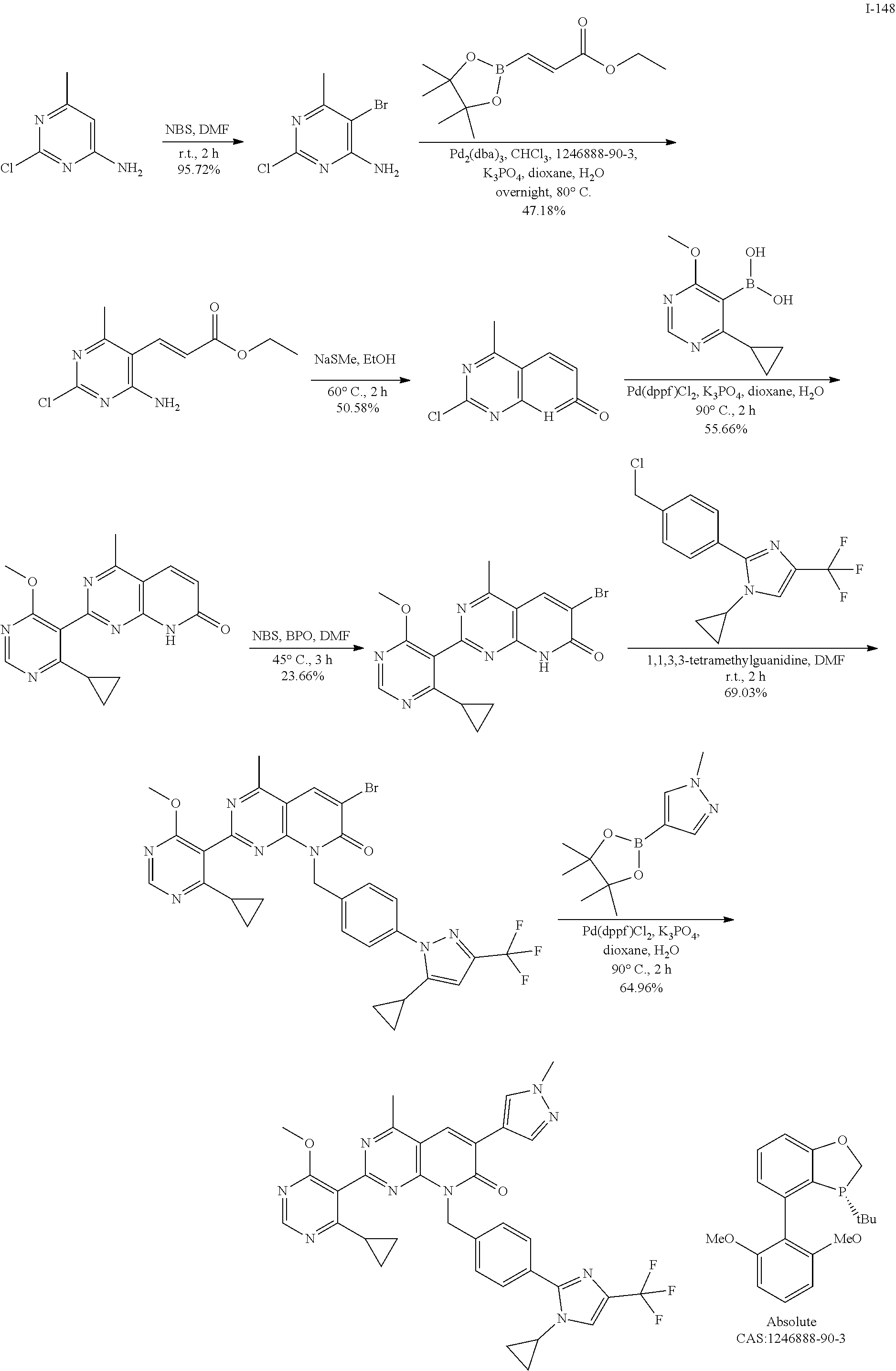

### Step 1: 5-Bromo-2-Chloro-6-Methylpyrimidin-4-Amine

A solution of 2-chloro-6-methylpyrimidin-4-amine (3 g, 20.896 mmol, 1 equiv) and NBS (5.58 g, 31.344 mmol, 1.5 equiv) in DMF (60 mL, 775.292 mmol, 37.10 equiv) was stirred for 2 h at room temperature under nitrogen atmosphere. The reaction was quenched by the addition of sat. Sodium thiosulfate (aq.) (100 mL) at 0° C. The resulting mixture was extracted with EtOAc (3×100 mL). The combined organic layers were washed with brine (3×50 mL), dried over anhydrous Na₂SO₄, filtered and concentrated under reduced pressure. The residue was purified by silica gel column chromatography, (eluting with PE/EA (1:1)) to afford 5-bromo-2-chloro-6-methylpyrimidin-4-amine (4.45 g, 95.72%) as an off-white solid.

LC-MS (ESI) m/z 222[M+H]

### Step 2: Ethyl (2E)-3-(4-Amino-2-Chloro-6-Methylpyrimidin-5-Yl)Prop-2-Enoate

To a stirred solution of 5-bromo-2-chloro-6-methylpyrimidin-4-amine (4 g, 17.980 mmol, 1 equiv) and ethyl (2E)-3-(4,4,5,5-tetramethyl-1,3,2-dioxaborolan-2-yl)prop-2-enoate (4.06 g, 17.980 mmol, 1 equiv) in dioxane (80 mL, 944.317 mmol, 52.52 equiv) were added K₃PO₄ (7.63 g, 35.960 mmol, 2 equiv) and H₂O (20 mL, 1110.186 mmol, 61.75 equiv) at room temperature under nitrogen atmosphere. To the above mixture was added 3-tert-butyl-4-(2,6-dimethoxyphenyl)-2,3-dihydro-1,3-benzoxaphosphole (0.4 g, 1.211 mmol, 0.07 equiv) and Pd₂(dba)₃. CHCl₃ (0.4 g, 0.386 mmol, 0.02 equiv) at room temperature. The resulting mixture was stirred for additional overnight at 80° C. The resulting mixture was filtered, the filter cake was washed with MeOH (3×100 mL), the filtrate was concentrated under reduced pressure. The residue was purified by reverse flash chromatography (C18 silica gel column, eluting with 0% to 100% MeCN/water (Water (10 mmol/L NH₄HCO₃)) in 10 min with UV detector (254 nm)) to afford ethyl (2E)-3-(4-amino-2-chloro-6-methylpyrimidin-5-yl)prop-2-enoate (2.05 g, 47.18%) as a yellow solid.

LC-MS (ESI) m/z 242 [M+H]

### Step 3: 2-Chloro-4-Methyl-811-Pyrido[2,3-d]Pyrimidin-7-One

A solution of ethyl (2E)-3-(4-amino-2-chloro-6-methylpyrimidin-5-yl)prop-2-enoate (1.7 g, 7.034 mmol, 1 equiv) and Sodiummethanethiolate (0.44 g, 6.331 mmol, 0.9 equiv) in EtOH (30 mL, 516.399 mmol, 73.41 equiv) was stirred for 2 h at 60° C. under nitrogen atmosphere. The resulting mixture was filtered, the filter cake was washed with EtOH (3×10 mL), the filtrate was concentrated under reduced pressure. The residue was purified by reverse flash chromatography (C18 silica gel column, eluting with 0% to 100% MeCN/water (Water (0.1% FA)) in 10 min with UV detector (254 nm)) to afford 2-chloro-4-methyl-8H-pyrido[2,3-d]pyrimidin-7-one (696 mg, 50.58%) as a yellow solid.

LC-MS (ESI) m/z 196 [M+H]

### Step 4: 2-(4-Cyclopropyl-6-Methoxypyrimidin-5-Yl)-4-Methyl-811-Pyrido[2,3-d]Pyrimidin-7-One

To a stirred solution of 2-chloro-4-methyl-8H-pyrido[2,3-d]pyrimidin-7-one (398.8 mg, 2.039 mmol, 1 equiv) and 4-cyclopropyl-6-methoxypyrimidin-5-ylboronic acid (395.5 mg, 2.039 mmol, 1 equiv) in dioxane (8 mL, 94.432 mmol, 46.32 equiv) were added K₃PO₄ (1.29 g, 6.117 mmol, 3 equiv) and H₂O (2 mL, 111.019 mmol, 54.45 equiv) at room temperature under nitrogen atmosphere. To the above mixture was added Pd(dppf)C12 (298.4 mg, 0.408 mmol, 0.2 equiv) at room temperature. The resulting mixture was stirred for additional 2 h at 90° C. The resulting mixture was filtered, the filter cake was washed with MeOH (3×15 mL), the filtrate was concentrated under reduced pressure. The residue was purified by reverse flash chromatography (C18 silica gel column, eluting with 0% to 100% MeCN/water (Water (0.1% FA)) in 10 min with UV detector (254 nm)) to afford 2-(4-cyclopropyl-6-methoxypyrimidin-5-yl)-4-methyl-8H-pyrido[2,3-d]pyrimidin-7-one (351 mg, 55.66%) as a yellow solid.

LC-MS (ESI) m/z 310 [M+H]

### Step 5: 6-Bromo-2-(4-Cyclopropyl-6-Methoxypyrimidin-5-Yl)-4-Methyl-811-Pyrido[2,3-d]Pyrimidin-7-One

To a stirred solution of 2-(4-cyclopropyl-6-methoxypyrimidin-5-yl)-4-methyl-8H-pyrido[2,3-d]pyrimidin-7-one (450 mg, 1.455 mmol, 1 equiv) and BPO (37.2 mg, 0.146 mmol, 0.1 equiv) in dimethylformamide (9 mL) were added NB S (776.7 mg, 4.365 mmol, 3 equiv) in portions at room temperature under nitrogen atmosphere. The resulting mixture was stirred for additional 3h at 45° C. The resulting mixture was concentrated under reduced pressure. The residue was purified by silica gel column chromatography, (eluting with PE/EA (1:1)) to afford 6-bromo-2-(4-cyclopropyl-6-methoxypyrimidin-5-yl)-4-methyl-8H-pyrido[2,3-d]pyrimidin-7-one (167 mg, 23.66%, purity:80%) as an off-white solid.

LC-MS (ESI) m/z 388 [M+H]

### Step 6: 6-Bromo-8-({4-[1-Cyclopropyl-4-(Trifluoromethyl)Imidazol-2-Yl]Phenyl}Methyl)-2-(4-Cyclopropyl-6-Methoxypyrimidin-5-Yl)-4-Methylpyrido[2,3-d]Pyrimidin-7-One

To a stirred solution of 6-bromo-2-(4-cyclopropyl-6-methoxypyrimidin-5-yl)-4-methyl-8H-pyrido[2,3-d]pyrimidin-7-one (167 mg, 0.344 mmol, 1 equiv, 80%) and 2-[4-(chloromethyl)phenyl]-1-cyclopropyl-4-(trifluoromethyl)imidazole (103.4 mg, 0.344 mmol, 1 equiv) in dimethylformamide (3 mL) were added 1,1,3,3-tetramethylguanidine (118.9 mg, 1.032 mmol, 3 equiv) at room temperature under nitrogen atmosphere. The resulting mixture was stirred for additional 2h at room temperature. The resulting mixture was filtered, the filter cake was washed with MeOH (3×3 mL), the filtrate was concentrated under reduced pressure. The residue was purified by reverse flash chromatography (C18 silica gel column, eluting with 0% to 100% MeCN/water (Water (0.1% FA)) in 10 min with UV detector (254 nm)) to afford 6-bromo-8-({4-[1-cyclopropyl-4-(trifluoromethyl)imidazol-2-yl]phenyl methyl)-2-(4-cyclopropyl-6-methoxypyrimidin-5-yl)-4-methylpyrido[2,3-d]pyrimidin-7-one (155 mg, 69.03%) as an orange solid.

LC-MS (ESI) m/z 652 [M+H]

### Step 7: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-4-Methyl-6-(1-Methylpyrazol-4-Yl)Pyrido[2,3-d]Pyrimidin-7-One

To a stirred solution of 6-bromo-8-({4-[1-cyclopropyl-4-(trifluoromethyl)imidazo]-2-yl]phenyl}methyl)-2-(4-cyclopropyl-6-methoxypyrimidin-5-yl)-4-methylpyrido[2,3-d]pyrimidin-7-one (155.2 mg, 0.238 mmol, 1 equiv) and 1-methyl-4-(4,4,5,5-tetramethyl-1,3,2-dioxaborolan-2-yl)pyrazole (59.4 mg, 0.286 mmol, 1.2 equiv) in dioxane (3 mL, mmol, 148.88 equiv) were added K₃PO₄ (126.2 mg, 0.595 mmol, 2.5 equiv) and H₂O (0.6 mL, 33.306 mmol, 140.02 equiv) at room temperature under nitrogen atmosphere. To the above mixture was added Pd(dppf)C12 (34.8 mg, 0.048 mmol, 0.2 equiv) at room temperature. The resulting mixture was stirred for additional 2 h at 90° C. The resulting mixture was filtered, the filter cake was washed with MeOH (3×5 mL), the filtrate was concentrated under reduced pressure. The residue was purified by reverse flash chromatography (C18 silica gel column, eluting with 0% to 100% MeCN/water (Water (0.1% FA)) in 10 min with UV detector (254 nm)) to afford 8-({4-[1-cyclopropyl-4-(trifluoromethyl)imidazo]-2-yl]phenyl}methyl)-2-(4-cyclopropyl-6-methoxypyrimidin-5-yl)-4-methyl-6-(1-methylpyrazol-4-yl)pyrido[2,3-d]pyrimidin-7-one (101 mg, 64.96%) as a brown yellow solid.

LC-MS (ESI) m/z 654 [M+H]

¹H NMR (400 MHz, DMSO-d6) δ 8.68 (s, 1H), 8.55 (s, 1H), 8.52 (s, 1H), 8.31 (s, 1H), 7.90 (s, 1H), 7.83-7.77 (m, 2H), 7.40 (m, 2H), 5.69 (s, 2H), 3.91 (s, 3H), 3.84 (s, 3H), 3.68 (dq, J=7.2, 3.6, 3.1 Hz, 1H), 2.90 (s, 3H), 1.72 (td, J=8.1, 4.2 Hz, 1H), 1.04-0.98 (m, 2H), 0.98-0.82 (m, 2H), 0.92-0.87 (m, 2H), 0.81-0.74 (m, 2H).

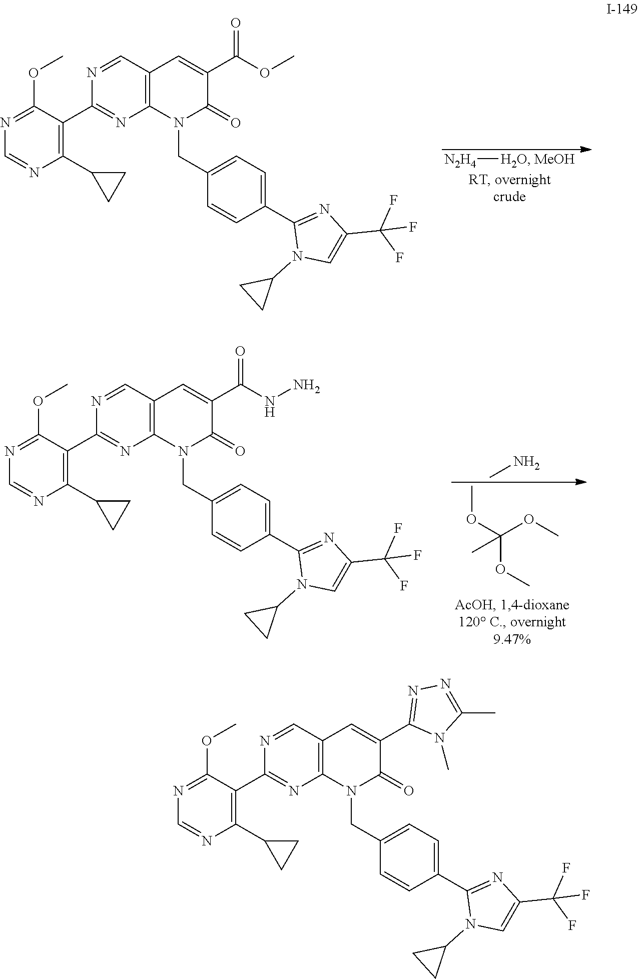

### Step 1: 8-({4-[1-cyclopropyl-4-(trifluoromethyl)imidazol-2-yl]phenyl}methyl)-2-(4-cyclopropyl-6-methoxypyrimidin-5-yl)-7-oxopyrido[2,3-d]pyrimidine-6-carbohydrazide

To a stirred solution of methyl 8-({4-[1-cyclopropyl-4-(trifluoromethyl)imidazo]-2-yl]phenyl}methyl)-2-(4-cyclopropyl-6-methoxypyrimidin-5-yl)-7-oxopyrido[2,3-d]pyrimidine-6-carboxylate (50 mg, 0.081 mmol, 1 equiv) in MeOH (1 mL) was added N₂H₄—H₂O (15 mg, 0.243 mmol, 3 equiv, 80%) at room temperature under nitrogen atmosphere. The resulting mixture was stirred for overnight at room temperature under nitrogen atmosphere. The reaction was quenched by the addition of Water (10 mL) at 0° C. The resulting mixture was extracted with EtOAc (3×mL). The combined organic layers were washed with brine (2×30 mL), dried over anhydrous Na₂SO₄. After filtration, the filtrate was concentrated under reduced pressure. This resulted in 8-({4-[1-cyclopropyl-4-(trifluoromethyl)imidazo]-2-yl]phenyl methyl)-2-(4-cyclopropyl-6-methoxypyrimidin-5-yl)-7-oxopyrido[2,3-d]pyrimidine-6-carbohydrazide (50 mg, crude) as a light yellow solid. The crude product was used in the next step directly without further purification.

LC-MS (ESI) m/z 618.20 [M+H]

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(4,5-Dimethyl-1,2,4-Triazol-3-Yl)Pyrido[2,3-d]Pyrimidin-7-One

To a stirred solution of 8-({4-[1-cyclopropyl-4-(trifluoromethyl)imidazol-2-yl]phenyl}methyl)-2-(4-cyclopropyl-6-methoxypyrimidin-5-yl)-7-oxopyrido[2,3-d]pyrimidine-6-carbohydrazide (50 mg, 0.081 mmol, 1 equiv) and 1,1,1-trimethoxyethane (14.5 mg, 0.121 mmol, 1.5 equiv) in 1,4-dioxane (1 mL) were added methylamine (0.1 mL, 0.243 mmol, 3 equiv, 2M in THF) and AcOH (0.2 mL) at room temperature under nitrogen atmosphere. The final reaction mixture was stirred for overnight at 120° C. The mixture was allowed to cool down to room temperature. The resulting mixture was diluted with water (10 mL). The resulting mixture was extracted with EtOAc (3×20 mL). The combined organic layers were washed with brine (2×mL), dried over anhydrous Na₂SO₄, filtered and concentrated under reduced pressure. The crude product (40 mg) was purified by Prep-HPLC with the following conditions (Column: Xselect CSH C18 OBD Column 30*150 mm 5 μm, n; Mobile Phase A: Water(0.1% FA), Mobile Phase B: ACN; Flow rate: 60 mL/min; Gradient: 28% B to 48% B in 8 min; Wave Length: 254 nm/220 nm; RT1(min): 8.53) to afford 8-({4-[1-cyclopropyl-4-(trifluoromethyl)imidazo]-2-yl]phenyl}methyl)-2-(4-cyclopropyl-6-methoxypyrimidin-5-yl)-6-(4, 5-dim ethyl-1,2,4-triazol-3 yl)pyrido[2,3-d]pyrimidin-7-one (5.1 mg, 9.47%, purity:98.4%) as an off-white solid.

LC-MS (ESI) m/z 655.25 [M+H]

¹H NMR (400 MHz, DMSO-d6) δ 9.44 (d, J=1.0 Hz, 1H), 8.71 (s, 1H), 8.51 (d, J=1.1 Hz, 1H), 7.91 (d, J=1.5 Hz, 1H), 7.82 (d, J=8.1 Hz, 2H), 7.45 (d, J=8.0 Hz, 2H), 5.65 (s, 2H), 3.85 (s, 3H), 3.71-3.67 (m, 1H), 3.46 (s, 3H), 2.42 (s, 3H), 1.80-1.75 (m, 1H), 1.04 (p, J=3.6 Hz, 2H), 0.95 (dd, J=7.4, 5.0 Hz, 2H), 0.89 (t, J=4.4 Hz, 2H), 0.79 (dq, J=7.5, 3.7 Hz, 2H).

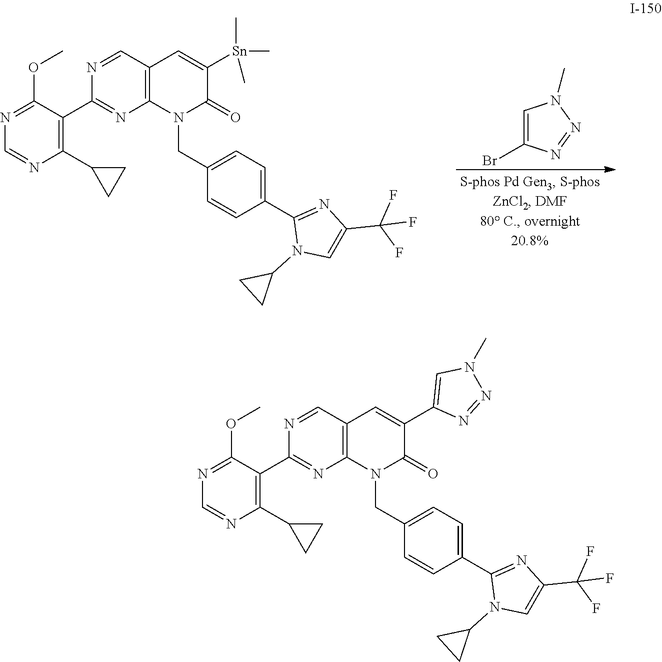

To a stirred solution of 8-({4-[1-cyclopropyl-4-(trifluoromethyl)imidazo]-2-yl]phenyl methyl)-2-(4-cyclopropyl-6-methoxypyrimidin-5-yl)-6-(trimethyl stannyl)pyrido[2,3-d]pyrimidin-7-one (100 mg, 0.138 mmol, 1 equiv) and 4-bromo-1-methyl-1,2,3-triazole (44.8 mg, mmol, 2 equiv) in DMF (2 mL) were added SPhos Pd Gen.3 (10 mg, 0.002 mmol, 0.09 equiv), S-phos (10 mg, 0.024 mmol, 0.18 equiv) and ZnCl₂ (18.8 mg, 0.138 mmol, 1 equiv) at room temperature under nitrogen atmosphere. The resulting mixture was stirred for overnight at 80° C. under nitrogen atmosphere. The mixture was allowed to cool down to room temperature. The resulting mixture was diluted with water (20 mL). The resulting mixture was extracted with EtOAc (3×50 mL). The combined organic layers were washed with brine (2×mL), dried over anhydrous Na₂SO₄, filtered and concentrated under reduced pressure. The crude product (50 mg) was purified by Prep-HPLC with the following conditions (Column: Sunfire prep C18 column, 30*150 mm, 51 μm; Mobile Phase A: Water(0.1% FA), Mobile Phase B: ACN; Flow rate: 60 mL/min; Gradient: 40% B to 60% B in 10 min; Wave Length: 254 nm/220 nm; RT1(min): 11.2) to afford 8-({4-[1-cyclopropyl-4-(trifluoromethyl)imidazol-2-yl]phenyl methyl)-2-(4-cyclopropyl-6-methoxypyrimidin-5-yl)-6-(1-methyl-1,2,3-triazol-4-yl)pyrido[2,3-d]pyrimidin-7-one (19.1 mg, 20.80%, purity:96.6%) as an off-white solid.

LC-MS (ESI) m/z 641.30 [M+H]

¹H NMR (400 MHz, DMSO-d6) δ 9.50 (s, 1H), 8.97 (s, 1H), 8.75 (s, 1H), 8.70 (s, 1H), 7.93-7.88 (m, 1H), 7.85-7.78 (m, 2H), 7.44 (d, J=8.3 Hz, 2H), 5.70 (s, 2H), 4.14 (s, 3H), 3.85 (s, 3H), 3.68 (tt, J=7.0, 3.9 Hz, 1H), 1.79 (tt, J=8.1, 4.6 Hz, 1H), 1.03 (dq, J=6.1, 3.4 Hz, 2H), 0.95 (dd, J=7.4, 5.1 Hz, 2H), 0.86 (dd, J=14.0, 7.1 Hz, 2H), 0.82-0.74 (m, 2H).

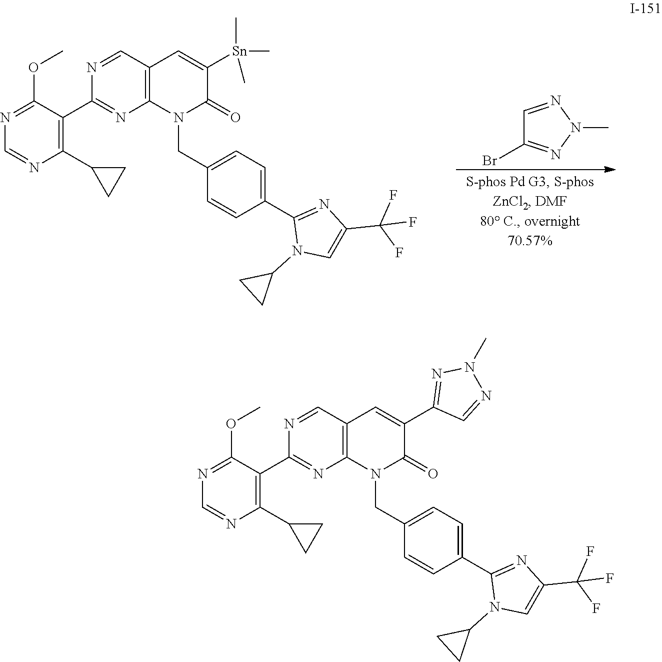

To a stirred solution of 8-({4-[1-cyclopropyl-4-(trifluoromethyl)imidazo]-2-yl]phenyl}methyl)-2-(4-cyclopropyl-6-methoxypyrimidin-5-yl)-6-(trimethylstannyl)pyrido[2,3-d]pyrimidin-7-one (50 mg, 0.069 mmol, 1 equiv) and 4-bromo-2-methyl-1,2,3-triazole (22.4 mg, mmol, 2 equiv) in DMF (1 mL) were added SPhos Pd Gen.3 (5 mg, 0.006 mmol, 0.09 equiv), S-phos (5 mg, 0.012 mmol, 0.18 equiv) and ZnCl₂ (9.4 mg, 0.069 mmol, 1 equiv) at room temperature under nitrogen atmosphere. The resulting mixture was stirred for overnight at ° C. under nitrogen atmosphere. The mixture was allowed to cool down to room temperature. The resulting mixture was diluted with water (20 mL). The resulting mixture was extracted with EtOAc (3×50 mL). The combined organic layers were washed with brine (2×20 mL), dried over anhydrous Na₂SO₄, filtered and The crude product (50 mg) was purified by Prep-HPLC with the following conditions (Column: Xselect CSH F-Phenyl OBD column 30*250 mm, 5 μm; Mobile Phase A: Water(0.1% FA), Mobile Phase B: MEOH; Flow rate: 60 mL/min; Gradient: 55% B to 73% B in 10 min; Wave Length: 254 nm/220 nm; RT1(min): 11.48) to afford 8-({4-[1-cyclopropyl-4-(trifluoromethyl)imidazo]-2-yl]phenyl}methyl)-2-(4-cyclopropyl-6-methoxypyrimidin-5-yl)-6-(2-methyl-1,2,3-triazol-4-yl)pyrido[2,3-d]pyrimidin-7-one (31.9 mg, as an off-white solid.

LC-MS (ESI) m/z 641.25 [M+H]

¹H NMR (400 MHz, DMSO-d6) δ 9.48 (s, 1H), 8.80 (s, 1H), 8.70 (s, 1H), 8.39 (s, 1H), 7.93-7.88 (m, 1H), 7.85-7.78 (m, 2H), 7.43 (d, J=8.3 Hz, 2H), 5.69 (s, 2H), 4.28 (s, 3H), 3.84 (s, 3H), 3.69 (tt, J=7.2, 4.0 Hz, 1H), 1.78 (tt, J=8.0, 4.5 Hz, 1H), 1.02 (p, J=3.6 Hz, 2H), (dd, J=7.4, 5.1 Hz, 2H), 0.93-0.83 (m, 2H), 0.78 (dq, J=6.7, 3.4 Hz, 2H).

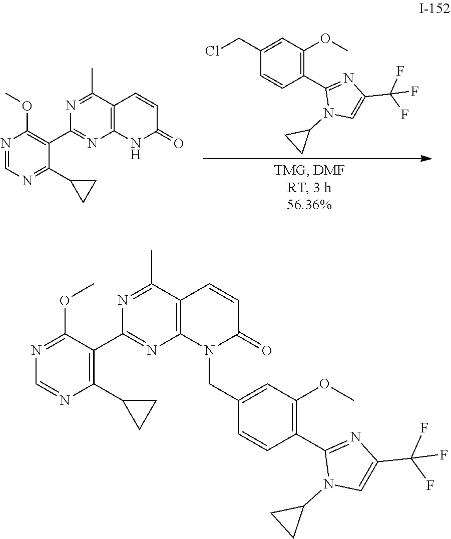 [SMILES: COc1ncnc(C2CC2)c1-c1nc(C)c2ccc(=O)[nH]c2n1 ; COc1cc(CCl)ccc1-c1nc(C(F)(F)F)cn1C1CC1 ; COc1cc(Cn2c(=O)ccc3c(C)nc(-c4c(OC)ncnc4C4CC4)nc32)ccc1-c1nc(C(F)(F)F)cn1C1CC1]

To a stirred solution of 2-(4-cyclopropyl-6-methoxypyrimidin-5-yl)-4-methyl-8H-pyrido[2,3-d]pyrimidin-7-one (110 mg, 0.356 mmol, 1 equiv) and 2-[4-(chloromethyl)-2-methoxyphenyl]-1-cyclopropyl-4-(trifluoromethyl)imidazole (105.8 mg, 0.320 mmol, 0.90 equiv) in DMF (2 mL) was added 1,1,3,3-Tetramethylguanidine (61.4 mg, 0.533 mmol, 1.50 equiv) at room temperature under nitrogen atmosphere. The resulting mixture was stirred for 3 h at room temperature under nitrogen atmosphere. The resulting mixture was filtered, the filter cake was washed with THF (3×1 mL). The resulting mixture was purified by reverse flash chromatography (C18 silica gel column, eluting with 0% to 100% MeCN/water (0.1% FA) in 10 min with UV detector (254 nm)) to afford 8-({4-[1-cyclopropyl-4-(trifluoromethyl)imidazo]-2-yl]-3-methoxyphenyl methyl)-2-(4-cyclopropyl-6-methoxypyrimidin-5-yl)-4-methylpyrido[2,3-d]pyrimidin-7-one (122.7 mg, 56.36%,purity:98.6%) as an off-white solid.

LC-MS (ESI) m/z 604.20 [M+H]

¹H NMR (400 MHz, DMSO-d6) δ 8.67 (s, 1H), 8.32 (d, J=9.8 Hz, 1H), 7.84 (d, J=1.4 Hz, 1H), 7.21 (d, J=7.8 Hz, 1H), 7.16 (d, J=1.5 Hz, 1H), 6.89 (d, J=9.8 Hz, 1H), 6.75 (dd, J=7.8, 1.5 Hz, 1H), 5.58 (s, 2H), 3.81 (s, 3H), 3.70 (s, 3H), 3.30 (dd, J=6.8, 4.5 Hz, 1H), 2.81 (s, 3H), 1.70 (tt, J=8.2, 4.6 Hz, 1H), 1.00 (dq, J=6.1, 3.4 Hz, 2H), 0.78 (dt, J=8.2, 3.4 Hz, 2H), 0.73 (d, J=5.6 Hz, 4H).

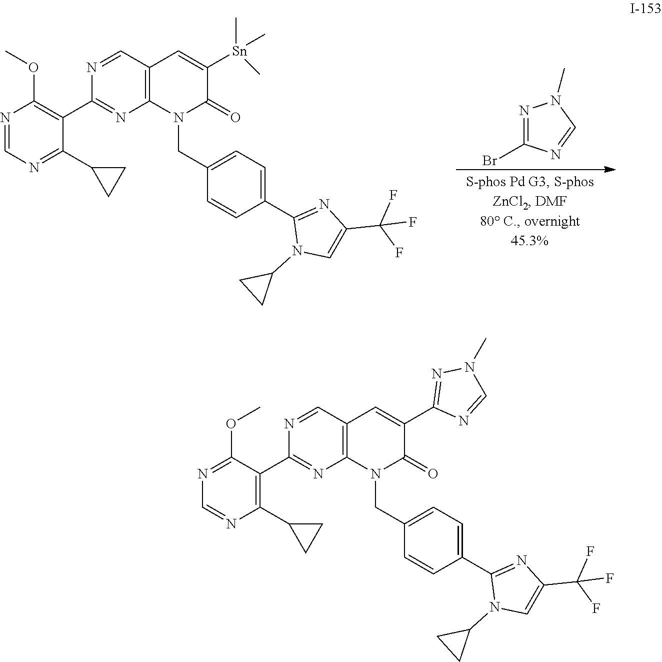

To a stirred mixture of 8-({4-[1-cyclopropyl-4-(trifluoromethyl)imidazo]-2-yl]phenyl methyl)-2-(4-cyclopropyl-6-methoxypyrimidin-5-yl)-6-(trimethyl stannyl)pyrido[2,3-d]pyrimidin-7-one (80 mg, 0.111 mmol, 1 equiv) and 3-bromo-1-methyl-1,2,4-triazole (35.9 mg, mmol, 2 equiv) in DMF (1.7 mL) were added S-phos (8 mg, 0.019 mmol, 0.18 equiv) and S-Phos Pd Gen.3 (8 mg, 0.010 mmol, 0.09 equiv) and ZnCl₂ (15.1 mg, 0.111 mmol, 1 equiv) at room temperature under nitrogen atmosphere. The resulting mixture was stirred for overnight at 80° C. under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×mL), dried over anhydrous Na₂SO₄, filtered and concentrated under reduced pressure. The crude product (85 mg) was purified by Prep-HPLC with the following conditions (Column:) (Bridge Prep Phenyl OBD Column 19*250 mm, 51 μm; Mobile Phase A: Water(0.1% FA), Mobile Phase B: ACN; Flow rate: 60 mL/min; Gradient: 31% B to 48% B in 10 min; Wave Length: 254 nm/220 nm; RT1(min): 10.38) to afford 8-({4-[1-cyclopropyl-4-(trifluoromethyl)imidazo]-2-yl]phenyl methyl)-2-(4-cyclopropyl-6-methoxypyrimidin-5-yl)-6-(1-methyl-1,2,4-triazol-3-yl)pyrido[2,3-d]pyrimidin-7-one (32.5 mg, 45.35%) as an off-white solid.

LC-MS (ESI) m/z 641.2 [M+H]

¹H NMR (400 MHz, DMSO-d6) δ 9.44 (s, 1H), 8.75 (s, 1H), 8.70 (s, 1H), 8.61 (s, 1H), 7.91 (s, 1H), 7.83 (d, J=8.1 Hz, 2H), 7.44 (d, J=8.2 Hz, 2H), 5.65 (s, 2H), 3.95 (s, 3H), 3.85 (s, 3H), 3.70 (tt, J=7.2, 4.0 Hz, 1H), 1.80 (tt, J=8.0, 4.5 Hz, 1H), 1.03 (p, J=3.6 Hz, 2H), (dd, J=7.5, 5.2 Hz, 2H), 0.91 (t, J=4.5 Hz, 2H), 0.79 (dq, J=6.9, 3.4 Hz, 2H).

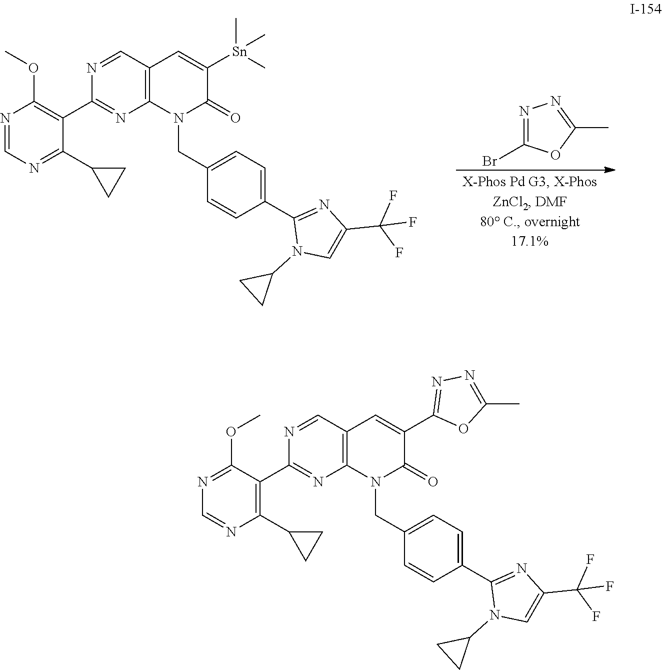

To a stirred mixture of 8-({4-[1-cyclopropyl-4-(trifluoromethyl)imidazo]-2-yl]phenyl methyl)-2-(4-cyclopropyl-6-methoxypyrimidin-5-yl)-6-(trimethyl stannyl)pyrido[2,3-d]pyrimidin-7-one (100 mg, 0.138 mmol, 1 equiv) and 2-bromo-5-methyl-1,3,4-oxadiazole (45.1 mg, 0.276 mmol, 2 equiv) in DMF (2 mL) were added X-Phos (10 mg, 0.021 mmol, 0.15 equiv), X-Phos Pd G3 (10 mg, 0.012 mmol, 0.09 equiv) and ZnCl₂ (18.87 mg, 0.138 mmol, 1 equiv) at room temperature under nitrogen atmosphere. The resulting mixture was stirred for overnight at 80° C. under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×mL), dried over anhydrous Na₂SO₄, filtered and concentrated under reduced pressure. The crude product (120 mg) was purified by Prep-HPLC with the following conditions (Column: Sunfire prep C18 column, 30*150 mm, 5 μm; Mobile Phase A: Water(0.1% FA), Mobile Phase B: ACN; Flow rate: 60 mL/min; Gradient: 37% B to 57% B in 10 min; Wave Length: 254 nm/220 nm; RT1(min): 11.53) to afford 8-({4-[1-cyclopropyl-4-(trifluoromethyl)imidazol-2-yl]phenyl methyl)-2-(4-cyclopropyl-6-methoxypyrimidin-5-yl)-6-(5-methyl-1,3,4-oxadiazol-2-yl)pyrido[2,3-d]pyrimidin-7-one (15.2 mg, 17.1%) as an off-white solid.

LC-MS (ESI) m/z 642.2 [M+H]

¹H NMR (400 MHz, DMSO-d6) δ 9.50 (s, 1H), 8.94 (s, 1H), 8.71 (s, 1H), 7.91 (s, 1H), 7.82 (d, J=8.1 Hz, 2H), 7.46 (d, J=8.1 Hz, 2H), 5.64 (s, 2H), 3.85 (s, 3H), 3.70 (s, 1H), 2.62 (s, 3H), 1.79 (d, J=7.8 Hz, 1H), 1.08-1.00 (m, 2H), 0.96 (d, J=7.2 Hz, 2H), 0.93-0.86 (m, 2H), 0.82-0.75 (m, 2H).

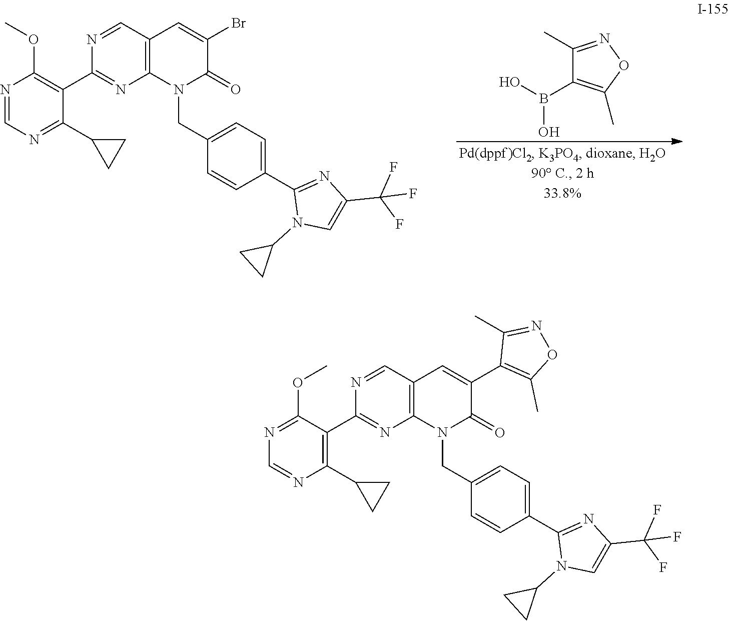

To a stirred mixture of 6-bromo-8-({4-[1-cyclopropyl-4-(trifluoromethyl)imidazo]-2-yl]phenyl methyl)-2-(4-cyclopropyl-6-methoxypyrimidin-5-yl)pyrido[2,3-d]pyrimidin-7-one (80 mg, 0.125 mmol, 1 equiv) and 3,5-dimethyl-1,2-oxazol-4-ylboronic acid (26.5 mg, 0.188 mmol, 1.5 equiv) in H₂O (0.3 mL) and dioxane (1.2 mL) were added Pd(dppf)Cl₂ (18.3 mg, 0.025 mmol, 0.2 equiv) and K₃PO₄ (66.5 mg, 0.313 mmol, 2.5 equiv) at room temperature under nitrogen atmosphere. The resulting mixture was stirred for 2 h at under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (90 mg) was purified by Prep-HPLC with the following conditions (Column: XBridge Prep OBD C18 Column 30*150 mm, 5 μm; Mobile Phase A: Water(10 mmol/L NH₄HCO₃), Mobile Phase B: ACN; Flow rate: 60 mL/min; Gradient: 43% B to 61% B in 10 min; Wave Length: 254 nm/220 nm; RT1(min): 9.43) to afford 8-({4-[1-cyclopropyl-4-(trifluoromethyl)imidazol-2-yl]phenyl methyl)-2-(4-cyclopropyl-6-methoxypyrimidin-5-yl)-6-(3,5-dimethyl-1,2-oxazol-4-yl)pyrido[2,3-d]pyrimidin-7-one (27.9 mg, 33.8%) as an off-white solid.

LC-MS (ESI) m/z 655.2 [M+H]

¹H NMR (400 MHz, DMSO-d6) δ 9.31 (s, 1H), 8.70 (s, 1H), 8.26 (s, 1H), 7.91 (d, J=1.4 Hz, 1H), 7.81 (d, J=8.2 Hz, 2H), 7.45 (d, J=8.1 Hz, 2H), 5.64 (s, 2H), 3.84 (s, 3H), 3.71 (d, J=4.1 Hz, 1H), 2.40 (s, 3H), 2.19 (s, 3H), 1.78-1.74 (m, 1H), 1.05-1.01 (m, 2H), 0.96 (d, J=6.8 Hz, 2H), 0.90 (d, J=4.4 Hz, 2H), 0.82-0.75 (m, 2H).

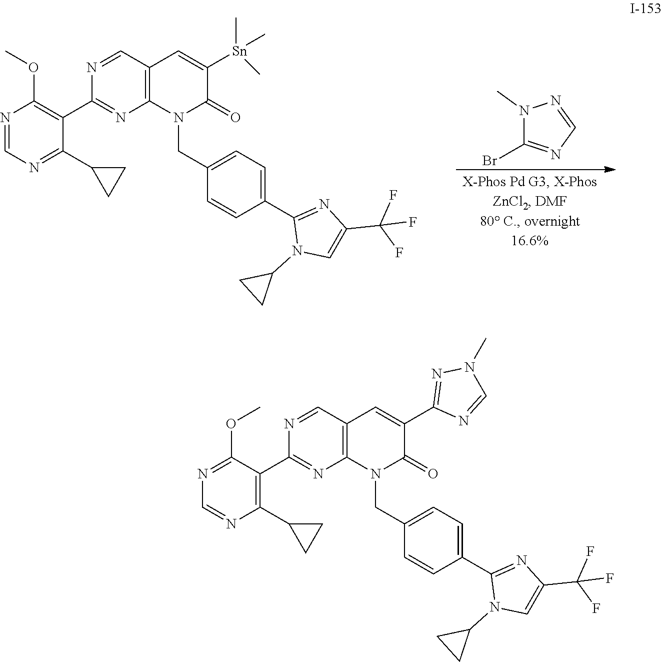

To a stirred mixture of 8-({4-[1-cyclopropyl-4-(trifluoromethyl)imidazo]-2-yl]phenyl methyl)-2-(4-cyclopropyl-6-methoxypyrimidin-5-yl)-6-(trimethyl stannyl)pyrido[2,3-d]pyrimidin-7-one (100 mg, 0.138 mmol, 1 equiv) and 5-bromo-1-methyl-1,2,4-triazole (44.8 mg, mmol, 2 equiv) in DMF (2 mL) were added X-phos Pd G3 (10 mg, 0.012 mmol, 0.09 equiv),X-phos (10 mg, 0.021 mmol, 0.15 equiv) and ZnCl₂ (18.9 mg, 0.138 mmol, 1 equiv) at room temperature under nitrogen atmosphere. The resulting mixture was stirred for overnight at 80° C. under nitrogen atmosphere. The mixture was allowed to cool down to room temperature. The resulting mixture was filtered, the filter cake was washed with THF (3×2 mL). The filtrate was concentrated under reduced pressure. The crude product (120 mg) was purified by Prep-HPLC with the following conditions (Column: XBridge Prep OBD C18 Column 30*150 mm, 51 μm; Mobile Phase A: Water(10 mmol/L NH₄HCO₃+0.1% NH₃.H₂O), Mobile Phase B: ACN; Flow rate: 60 mL/min; Gradient: 34% B to 52% B in 10 min; Wave Length: 254 nm/220 nm; RT1(min): 10.38) to afford 8-({4-[1-cyclopropyl-4-(trifluoromethyl)imidazol-2-yl]phenyl methyl)-2-(4-cyclopropyl-6-methoxypyrimidin-5-yl)-6-(2-methyl-1,2,4-triazol-3-yl)pyrido[2,3-d]pyrimidin-7-one (15.1 mg, 16.6%) as an off-white solid.

LC-MS (ESI) m/z 641.2 [M+H]

¹H NMR (400 MHz, DMSO-d6) δ 9.44 (s, 1H), 8.71 (s, 1H), 8.57 (s, 1H), 8.08 (s, 1H), 7.91 (d, J=1.4 Hz, 1H), 7.82 (d, J=8.3 Hz, 2H), 7.46 (d, J=8.2 Hz, 2H), 5.67 (s, 2H), 3.83 (d, J=13.0 Hz, 6H), 3.70 (tt, J=7.4, 4.0 Hz, 1H), 1.78 (tt, J=8.4, 4.7 Hz, 1H), 1.04 (t, J=3.8 Hz, 2H), 1.00-0.85 (m, 4H), 0.79 (dd, J=7.8, 3.4 Hz, 2H).

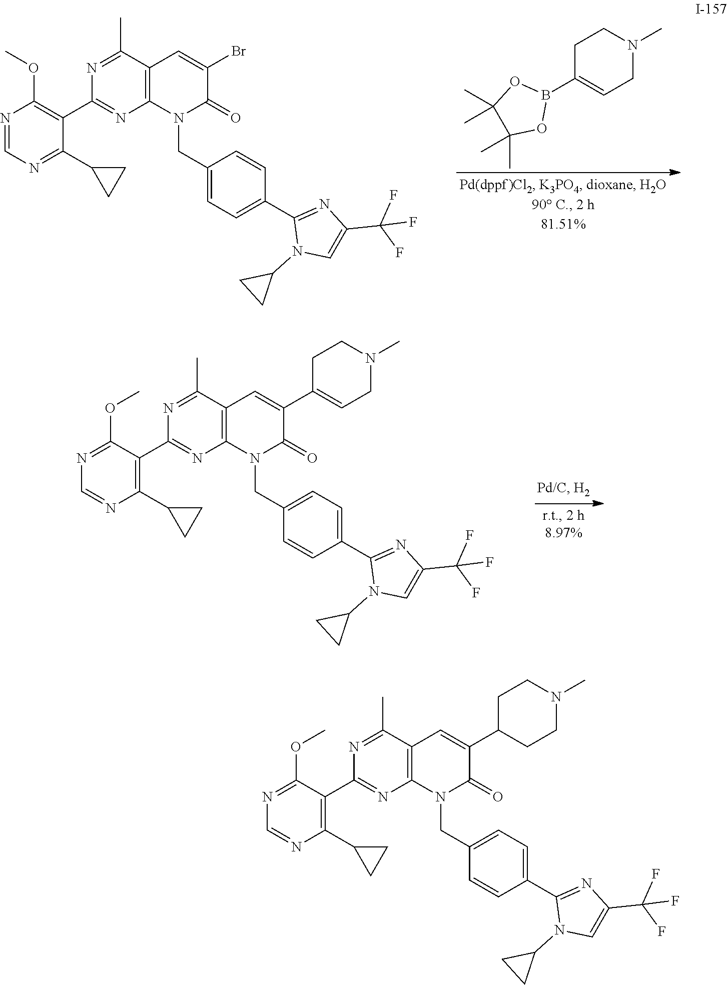

### Step 1: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-4-Methyl-6-(1-Methyl-3,6-Dihydro-211-Pyridin-4-Yl)Pyrido[2,3-d]Pyrimidin-7-One

To a stirred solution of 6-bromo-8-({4-[1-cyclopropyl-4-(trifluoromethyl)imidazo]-2-yl]phenyl}methyl)-2-(4-cyclopropyl-6-methoxypyrimidin-5-yl)-4-methylpyrido[2,3-d]pyrimidin-7-one (237 mg, 0.363 mmol, 1 equiv) and 1-methyl-4-(4,4,5,5-tetramethyl-1,3,2-dioxaborolan-2-yl)-3,6-dihydro-2H-pyridine (121.6 mg, 0.545 mmol, 1.50 equiv) in dioxane (5 mL) and H₂O (1 mL) were added K₃PO₄ (192.8 mg, 0.908 mmol, 2.50 equiv) and Pd(dppf)C12 (53.2 mg, 0.073 mmol, 0.20 equiv) at room temperature. The resulting mixture was stirred for 2 h at 90° C. under nitrogen atmosphere. The resulting mixture was filtered, the filter cake was washed with MeCN (3×5 mL), the filtrate was concentrated under reduced pressure. The residue was purified by reverse flash chromatography (C18 silica gel column, eluting with 0% to 100% MeCN/water (0.1% FA) in 10 min with UV detector (254 nm)) to afford 8-({4-[1-cyclopropyl-4-(trifluoromethyl)imidazo]-2-yl]phenyl}methyl)-2-(4-cyclopropyl-6-methoxypyrimidin-5-yl)-4-methyl-6-(1-methyl-3,6-dihydro-2H-pyridin-4-yl)pyrido[2,3-d]pyrimidin-7-one (198 mg, 81.51%) as a light yellow solid.

LC-MS (ESI) m/z 669 [M+H]

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-4-Methyl-6-(1-Methylpiperidin-4-Yl)Pyrido[2,3-d]Pyrimidin-7-One

To a solution of 8-({4-[1-cyclopropyl-4-(trifluoromethyl)imidazo]-2-yl]phenyl}methyl)-2-(4-cyclopropyl-6-methoxypyrimidin-5-yl)-4-methyl-6-(1-methyl-3,6-dihydro-2H-pyridin-4-yl)pyrido[2,3-d]pyrimidin-7-one (50 mg, 0.075 mmol, 1 equiv) in 3 mL MeOH was added 10% Pd/C (50 mg) under nitrogen atmosphere in a mL round-bottom flask. The mixture was hydrogenated at room temperature for 2 h under hydrogen atmosphere using a hydrogen balloon, filtered through a Celite pad and concentrated under reduced pressure. The crude product (50 mg) was purified by Prep-HPLC with the following conditions (Column:) (Bridge Prep Phenyl OBD Column 19*250 mm, 51 μm; Mobile Phase A: Water(0.1% FA), Mobile Phase B: ACN; Flow rate: 60 mL/min; Gradient: 29% B to 44% B in 10 min; Wave Length: 254 nm/220 nm; RT1(min): 7.06) to afford 8-({4-[1-cyclopropyl-4-(trifluoromethyl)imidazo]-2-yl]phenyl methyl)-2-(4-cyclopropyl-6-methoxypyrimidin-5-yl)-4-methyl-6-(1-methylpiperidin-4-yl)pyrido[2,3-d]pyrimidin-7-one (4.5 mg, 8.97%) as an off-white solid.

LC-MS (ESI) m/z 671[M+H]

¹H NMR (400 MHz, DMSO-d6) δ 8.67 (s, 1H), 8.30 (s, 0.819H, formate), 7.97 (s, 1H), 7.89 (s, 1H), 7.79 (d, J=8.3 Hz, 2H), 7.37 (d, J=8.2 Hz, 2H), 5.62 (s, 2H), 3.82 (s, 3H), 3.68 (dt, J=7.1, 3.5 Hz, 1H), 3.48-3.25 (m, 3H, impurities), 3.06 (m, 2H), 2.91-2.84 (m, 4H), 2.36 (s, 3H), 2.29 (m, 2H), 1.91-1.68 (m, 4H), 1.67 (td, J=8.0, 4.0 Hz, 1H), 1.04-0.90 (m, 4H), (q, J=3.8 Hz, 2H), 0.76 (dt, J=6.9, 3.5 Hz, 2H).

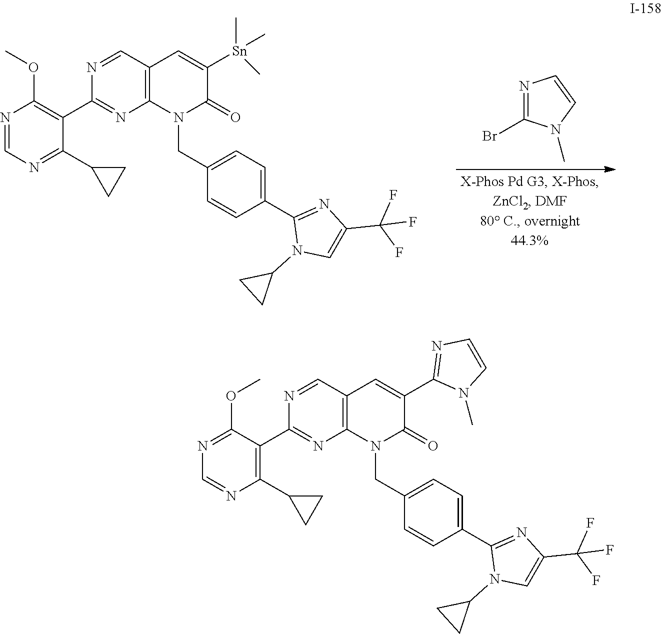

To a stirred mixture of 8-({4-[1-cyclopropyl-4-(trifluoromethyl)imidazo]-2-yl]phenyl methyl)-2-(4-cyclopropyl-6-methoxypyrimidin-5-yl)-6-(trimethyl stannyl)pyrido[2,3-d]pyrimidin-7-one (80 mg, 0.111 mmol, 1 equiv) and 2-bromo-1-methylimidazole (35.6 mg, 0.222 mmol, 2 equiv) in DMF (1.6 mL) were added X-Phos (8 mg, 0.017 mmol, 0.15 equiv), X-Phos Pd G3 (8 mg, 0.009 mmol, 0.09 equiv) and ZnCl₂ (15.1 mg, 0.111 mmol, 1 equiv) at room temperature under nitrogen atmosphere. The resulting mixture was stirred for overnight at under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (100 mg) was purified by Prep-HPLC with the following conditions (Column: Sunfire prep C18 column, 30*150 mm, 51 μm; Mobile Phase A: Water(0.1% FA), Mobile Phase B: ACN; Flow rate: mL/min; Gradient: 10% B to 30% B in 10 min; Wave Length: 254 nm/220 nm; RT1(min): 13.53) to afford 8-({4-[1-cyclopropyl-4-(trifluoromethyl)imidazol-2-yl]phenyl methyl)-2-(4-cyclopropyl-6-methoxypyrimidin-5-yl)-6-(1-methylimidazol-2-yl)pyrido[2,3-d]pyrimidin-7-one (31.5 mg, 44.3%) as an off-white solid.

LC-MS (ESI) m/z 640.2 [M+H]

¹H NMR (400 MHz, DMSO-d6) δ 9.40 (s, 1H), 8.71 (s, 1H), 8.42 (s, 1H), 7.91 (d, J=1.5 Hz, 1H), 7.82 (d, 2H), 7.44 (d, J=8.1 Hz, 2H), 7.33 (s, 1H), 7.03 (s, 1H), 5.66 (s, 2H), 3.85 (s, 3H), 3.69 (tt, J=7.5, 4.1 Hz, 1H), 3.59 (s, 3H), 1.77 (tt, J=8.4, 4.6 Hz, 1H), 1.03 (p, J=3.7 Hz, 2H), 0.95 (qd, J=7.4, 3.7 Hz, 2H), 0.88 (dq, J=7.3, 3.8 Hz, 2H), 0.79 (dq, J=7.0, 3.5 Hz, 2H).

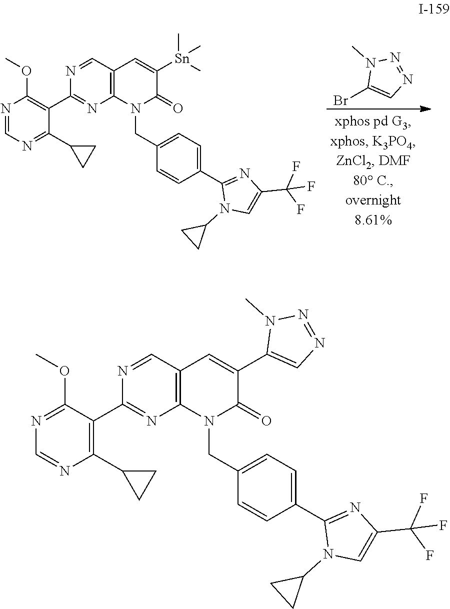

To a stirred solution of 8-({4-[1-cyclopropyl-4-(trifluoromethyl)imidazo]-2-yl]phenyl}methyl)-2-(4-cyclopropyl-6-methoxypyrimidin-5-yl)-6-(trimethylstannyl)pyrido[2,3-d]pyrimidin-7-one (90 mg, 0.125 mmol, 1 equiv) and 5-bromo-1-methyl-1,2,3-triazole (40.37 mg, mmol, 2 equiv) in DMF (2 mL) were added Xphos Pd G3 (9 mg, 0.011 mmol, 0.09 equiv), Xphos (9 mg, 0.019 mmol, 0.15 equiv) and ZnCl₂ (17 mg, 0.125 mmol, 1.00 equiv) at room temperature under nitrogen atmosphere. The resulting mixture was stirred for overnight at ° C. under nitrogen atmosphere. The mixture was allowed to cool down to room temperature. The resulting mixture was diluted with water (20 mL). The resulting mixture was extracted with EtOAc (3×20 mL). The combined organic layers were washed with brine (2×10 mL), dried over anhydrous Na₂SO₄, filtered and concentrated under reduced pressure. The crude product (40 mg) was purified by Prep-HPLC with the following conditions (Column: YMC-Actus Triart C18 ExRS 30*150 mm, 5 μm; Mobile Phase A: Water(10 mmol/L NH 4 HCO 3), Mobile Phase B: ACN; Flow rate: 60 mL/min; Gradient: 41% B to 58% B in 10 min; Wave Length: 254 nm/220 nm; RT1(min): 10.23) to afford 8-({4-[1-cyclopropyl-4-(trifluoromethyl)imidazol-2-yl]phenyl}methyl)-2-(4-cyclopropyl-6-methoxypyrimidin-5-yl)-6-(3-methyl-1,2,3-triazol-4-yl)pyrido[2,3-d]pyrimidin-7-one (6.9 mg, 8.61%, purity:99.6%) as an off-white solid.

LC-MS (ESI) m/z 641.25 [M+H]

¹H NMR (400 MHz, DMSO-d6) δ 9.38 (s, 1H), 8.71 (s, 1H), 8.49 (s, 1H), 7.99 (s, 1H), 7.91 (d, J=1.4 Hz, 1H), 7.85-7.78 (m, 2H), 7.45 (d, J=8.3 Hz, 2H), 5.65 (s, 2H), 4.06 (s, 3H), 3.84 (s, 3H), 3.69 (tq, J=9.9, 6.0, 5.0 Hz, 1H), 1.76 (tt, J=8.2, 4.7 Hz, 1H), 1.03 (t, J=3.9 Hz, 2H), 0.95 (dd, J=7.5, 4.9 Hz, 2H), 0.91-0.86 (m, 2H), 0.79 (dq, J=7.1, 3.5 Hz, 2H).

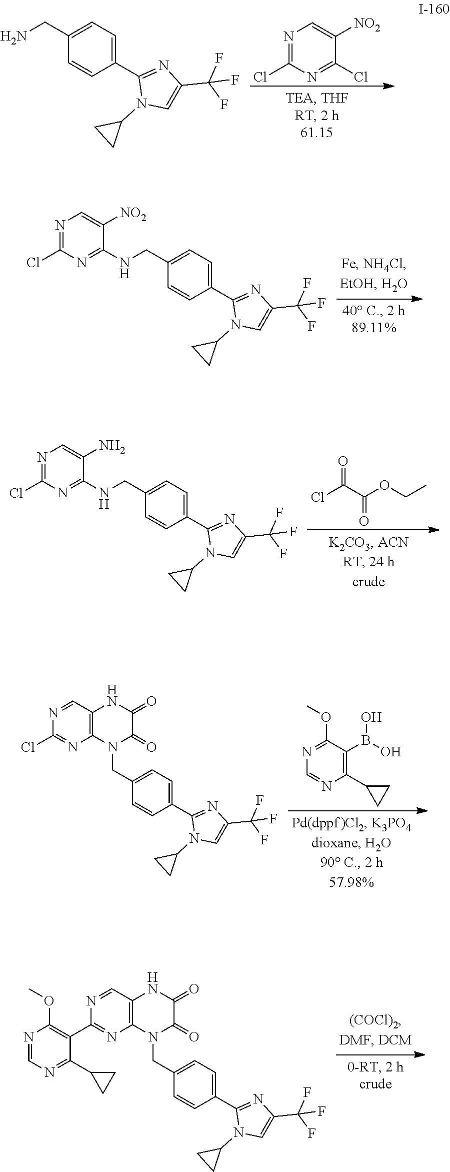

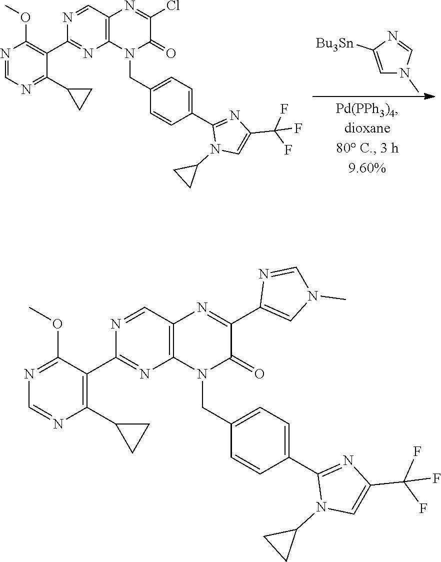

### Step 1: 2-Chloro-N-({4-[1-Cyclopropyl-4-(Trifluoromethyl)Imidazol-2-Yl]Phenyl}Methyl)-5-Nitropyrimidin-4-Amine

To a stirred solution of 1-{4-[1-cyclopropyl-4-(trifluoromethyl)imidazo]-2-yl]phenyl}methanamine (1 g, 3.555 mmol, 1 equiv) and TEA (1.08 g, 10.665 mmol, 3 equiv) in THF (20 mL) was added 2,4-dichloro-5-nitropyrimidine (1.38 g, 7.110 mmol, 2 equiv) at room temperature. The resulting mixture was stirred for 2 h at room temperature. The resulting mixture was concentrated under reduced pressure. The residue was purified by reversed-phase flash chromatography (C18 silica gel column, eluting with 50% to 80% MeCN/water (0.1% FA) in 10 min with UV detector (254 nm)) to afford 2-chloro-N-({4-[1-cyclopropyl-4-(trifluoromethyl)imidazo]-2-yl]phenyl}methyl)-5-nitropyrimidin-4-amine (954 mg, 61.15%) as a yellow oil.

LC-MS (ESI) m/z 439.0 [M+H]

### Step 2: 2-Chloro-N4-({4-[1-Cyclopropyl-4-(Trifluoromethyl)Imidazol-2-Yl]Phenyl}Methyl)Pyrimidine-4,5-Diamine

To a stirred solution of 2-chloro-N-({4-[1-cyclopropyl-4-(trifluoromethyl)imidazo]-2-yl]phenyl}methyl)-5-nitropyrimidin-4-amine (954 mg, 2.174 mmol, 1 equiv) in EtOH (20 mL) and H₂O (4 mL) were added Fe (0.97 g, 17.392 mmol, 8 equiv) and NH₄Cl (0.58 g, 10.870 mmol, 5 equiv) at room temperature. The resulting mixture was stirred for 2 h at 40° C. under nitrogen atmosphere. The resulting mixture was filtered, the filter cake was washed with THF (3×30 mL). The filtrate was concentrated under reduced pressure. The residue was purified by silica gel column chromatography (eluting with PE/EA (1:2)) to afford 2-chloro-N4-({4-[1-cyclopropyl-4-(trifluoromethyl)imidazol-2-yl]phenyl methyl)pyrimidine-4,5-diamine (792 mg, 89.11%) as a reddish brown solid.

LC-MS (ESI) m/z 409.1 [M+H]

### Step 3: 2-Chloro-8-({4-[1-Cyclopropyl-4-(Trifluoromethyl)Imidazol-2-Yl]Phenyl}Methyl)-511-Pteridine-6,7-Dione

To a stirred solution of 2-chloro-N4-({4-[1-cyclopropyl-4-(trifluoromethyl)imidazo]-2-yl]phenyl}methyl)pyrimidine-4,5-diamine (792 mg, 1.937 mmol, 1 equiv) and ethyl chloroglyoxylate (529.0 mg, 3.875 mmol, 2.00 equiv) in ACN (20 mL) was added K₂CO₃ (669.4 mg, 4.844 mmol, 2.50 equiv) at room temperature. The resulting mixture was stirred for overnight at room temperature. The resulting mixture was diluted with water (15 mL). The resulting mixture was extracted with EtOAc (3×15 mL). The combined organic layers were washed with brine (2×30 mL), dried over anhydrous Na₂SO₄, filtered and concentrated under reduced pressure to afford 2-chloro-8-({4-[1-cyclopropyl-4-(trifluoromethyl)imidazol-2-yl]phenyl}methyl)-5H-pteridine-6,7-dione (970 mg, crude) as a reddish brown solid.

LC-MS (ESI) m/z 463.0 [M+H]

### Step 4: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-511-Pteridine-6,7-Dione

To a stirred solution of 2-chloro-8-({4-[1-cyclopropyl-4-(trifluoromethyl)imidazo]-2-yl]phenyl}methyl)-5H-pteridine-6,7-dione (1.8 g, 3.889 mmol, 1 equiv) and 4-cyclopropyl-6-methoxypyrimidin-5-ylboronic acid (1.13 g, 5.833 mmol, 1.50 equiv) in dioxane (30 mL) and H₂O (6 mL) were added K₃PO₄ (2.06 g, 9.723 mmol, 2.50 equiv) and Pd(dppf)Cl₂ (0.57 g, 0.778 mmol, 0.20 equiv) at room temperature. The resulting mixture was stirred for 2 h at 90° C. under nitrogen atmosphere. The resulting mixture was filtered, the filter cake was washed with THF (3×30 mL). The filtrate was concentrated under reduced pressure. The residue was purified by reversed-phase flash chromatography (C18 silica gel column, eluting with 40% to 60% MeCN/water (0.1% FA) in 10 min with UV detector (254 nm)) to afford 8-({4-[1-cyclopropyl-4-(trifluoromethyl)imidazol-2-yl]phenyl methyl)-2-(4-cyclopropyl-6-methoxypyrimidin-5-yl)-5H-pteridine-6,7-dione (1.3 g, 57.98%) as a light yellow solid.

LC-MS (ESI) m/z 577.1 [M+H]

### Step 5: 6-Chloro-8-({4-[1-Cyclopropyl-4-(Trifluoromethyl)Imidazol-2-Yl]Phenyl}Methyl)-2-(4-Cyclopropyl-6-Methoxypyrimidin-5-Yl)Pteridin-7-One

To a stirred solution of 8-({4-[1-cyclopropyl-4-(trifluoromethyl)imidazo]-2-yl]phenyl}methyl)-2-(4-cyclopropyl-6-methoxypyrimidin-5-yl)-5H-pteridine-6,7-dione (100 mg, mmol, 1 equiv) in DCM (4 mL) were added (COCl)₂ (66.0 mg, 0.520 mmol, 3.00 equiv) at under nitrogen atmosphere. Then, to the above mixture was added DMF (12.8 mg, 0.175 mmol, 1.01 equiv) at 0° C. The resulting mixture was stirred for additional 2 h at room temperature. The resulting mixture was concentrated under reduced pressure to afford 6-chloro-8-({4-[1-cyclopropyl-4-(trifluoromethyl)imidazo]-2-yl]phenyl methyl)-2-(4-cyclopropyl-6-methoxypyrimidin-5-yl)pteridin-7-one (100 mg, crude) as a brown solid.

LC-MS (ESI) m/z 595.1 [M+H]

### Step 6: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-6-(1-Methylimidazol-4-Yl)Pteridin-7-One

To a stirred solution of 6-chloro-8-({4-[1-cyclopropyl-4-(trifluoromethyl)imidazo]-2-yl]phenyl methyl)-2-(4-cyclopropyl-6-methoxypyrimidin-5-yl)pteridin-7-one (30 mg, 0.050 mmol, 1 equiv) and 1-methyl-4-(tributylstannyl)imidazole (37.5 mg, 0.100 mmol, 2 equiv) in dioxane (2 mL) was added Pd(PPh₃)₄ (5.9 mg, 0.005 mmol, 0.1 equiv) at room temperature. The resulting mixture was stirred for 3 h at 80° C. under nitrogen atmosphere. The mixture was purified by reversed-phase flash chromatography (C18 silica gel column, eluting with 0% to 100% MeCN/water (10 mmol/L NH₄HCO₃) in 10 min with UV detector (254 nm)) to afford the crude product. The crude product was purified by Prep-HPLC with the following conditions (Column: Xselect CSH C18 OBD Column 30*150 mm 5 μm; Mobile Phase A: Water(0.1% FA), Mobile Phase B: ACN; Flow rate: 60 mL/min; Gradient: 21% B to 41% B in 8 min; Wave Length: 254 nm/220 nm; RT1(min): 9.28) to afford 8-({4-[1-cyclopropyl-4-(trifluoromethyl)imidazol-2-yl]phenyl methyl)-2-(4-cyclopropyl-6-methoxypyrimidin-5-yl)-6-(1-methylimidazol-4-yl)pteridin-7-one (3.1 mg, 9.60%) as a light yellow solid.

LC-MS (ESI) m/z 641.2 [M+H]

¹H NMR (400 MHz, DMSO-d6) δ 9.35 (s, 1H), 8.70 (s, 1H), 8.46 (s, 1H), 7.96-7.77 (m, 4H), 7.49 (d, J=8.1 Hz, 2H), 5.61 (s, 2H), 3.86 (s, 3H), 3.78 (s, 3H), 3.69 (tt, J=7.1, 3.9 Hz, 1H), 1.87-1.73 (m, 1H), 1.24 (s, OH), 1.03 (p, J=3.6 Hz, 2H), 0.95 (dd, J=7.3, 5.0 Hz, 2H), −0.84 (m, 2H), 0.80 (dq, J=7.0, 3.5 Hz, 2H).

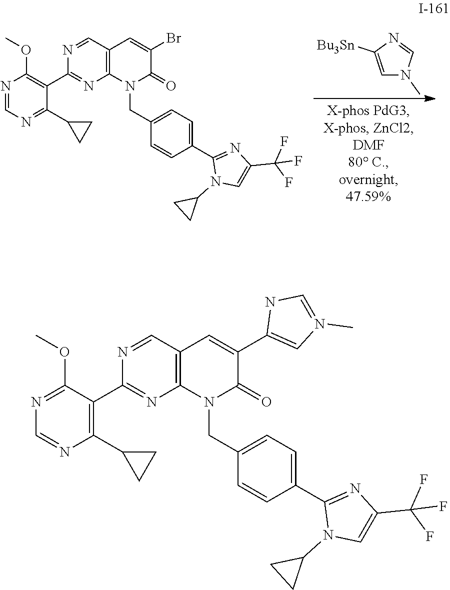

A solution of 6-bromo-8-({4-[1-cyclopropyl-4-(trifluoromethyl)imidazo]-2-yl]phenyl}methyl)-2-(4-cyclopropyl-6-methoxypyrimidin-5-yl)pyrido[2,3-d]pyrimidin-7-one (50 mg, 0.078 mmol, 1 equiv),1-methyl-4-(tributylstannyl)imidazole (50 mg, 0.135 mmol, 1.72 equiv), X-phos (5 mg, 0.010 mmol, 0.13 equiv), ZnCl₂ (10 mg, 0.073 mmol, 0.94 equiv) and XPhos Pd G3 (5 mg, 0.006 mmol, 0.08 equiv) in DMF (5.00 mL, 64.348 mmol, 824.97 equiv) was stirred for overnight at 80° C. under nitrogen atmosphere. The mixture was allowed to cool down to room temperature. The crude product (70 mg) was purified by Prep-HPLC with the following conditions (Column: Xselect CSH F-Phenyl OBD column 30*250 mm, 5 μm; Mobile Phase A: Water(0.1% FA), Mobile Phase B: ACN; Flow rate: 60 mL/min; Gradient: 21% B to 39% B in 10 min; Wave Length: 254 nm/220 nm; RT1(min): 12.75) to afford 8-({4-[1-cyclopropyl-4-(trifluoromethyl)imidazo]-2-yl]phenyl methyl)-2-(4-cyclopropyl-6-methoxypyrimidin-5-yl)-6-(1-methylimidazol-4-yl)pyrido[2,3-d]pyrimidin-7-one (24.2 mg, 47.59%) as an off-white solid.

LC-MS (ESI) m/z 640.25 [M+H].

¹H NMR (400 MHz, DMSO-d6) δ 9.42 (s, 1H), 8.70 (dd, J=7.9, 1.8 Hz, 2H), 8.04 (s, 1H), 7.90 (s, 1H), 7.86-7.77 (m, 3H), 7.42 (d, J=8.0 Hz, 2H), 5.70 (s, 2H), 3.84 (d, J=1.9 Hz, 3H), 3.74 (d, J=2.0 Hz, 3H), 3.68 (m, 1H), 1.78 (m, 1H), 1.02 (m, 2H), 0.96 (d, J=7.2 Hz, 2H), 0.90 (m, 2H), 0.84-(m, 2H).

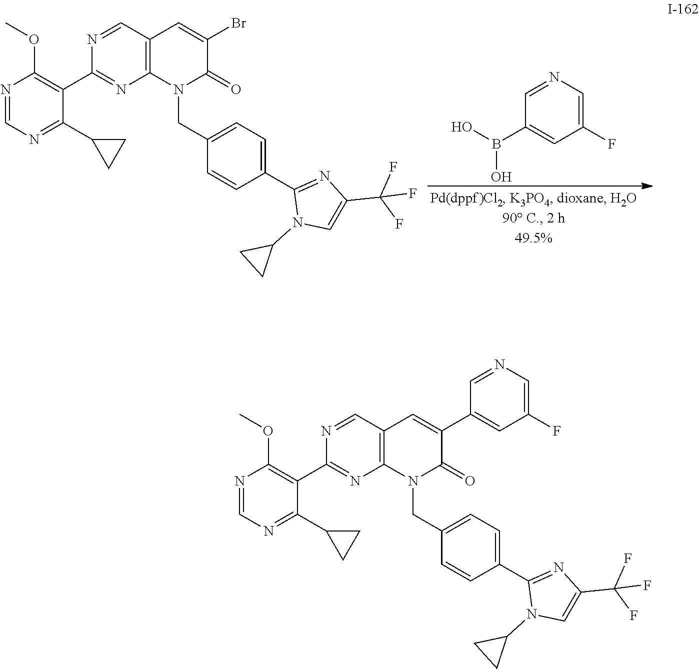 [SMILES: COc1ncnc(C2CC2)c1-c1ncc2cc(Br)c(=O)n(Cc3ccc(-c4nc(C(F)(F)F)cn4C4CC4)cc3)c2n1 ; OB(O)c1cncc(F)c1 ; COc1ncnc(C2CC2)c1-c1ncc2cc(-c3cncc(F)c3)c(=O)n(Cc3ccc(-c4nc(C(F)(F)F)cn4C4CC4)cc3)c2n1]

To a stirred mixture of 6-bromo-8-({4-(1-cyclopropyl-4-yl)pyrido[2,3-d]pyrimidin-7-one (80 mg, 0.125 mmol, 1 equiv) and 5-fluoropyridin-3-ylboronic acid (26.5 mg, 0.188 mmol, 1.5 equiv) in H₂O (0.3 mL) and dioxane (1.2 mL) were added Pd(dppf)Cl₂ (18.3 mg, 0.025 mmol, 0.2 equiv) and K₃PO₄ (66.5 mg, 0.313 mmol, 2.5 equiv) at room temperature under nitrogen atmosphere. The resulting mixture was stirred for 2h at under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (85 mg) was purified by Prep-HPLC with the following conditions (Column: Sunfire prep C18 column, 30*150 mm, 5 m; Mobile Phase A: Water(0.1% FA), Mobile Phase B: ACN; Flow rate: mL/min; Gradient: 46% B to 66% B in 10 min; Wave Length: 254 nm/220 nm; RT1(min): 10.87) to afford 8-({4-[1-cyclopropyl-4-(trifluoromethyl)imidazol-2-yl]phenyl methyl)-2-(4-cyclopropyl-6-methoxypyrimidin-5-yl)-6-(5-fluoropyridin-3-yl)pyrido[2,3-d]pyrimidin-7-one (40.7 mg, 49.5%) as an off-white solid.

LC-MS (ESI) m/z 655.2 [M+H]

¹H NMR (400 MHz, DMSO-d6) δ 9.35 (s, 1H), 8.83 (d, J=1.8 Hz, 1H), 8.71 (s, 1H), 8.67 (d, J=2.8 Hz, 1H), 8.58 (s, 1H), 8.13 (dt, J=10.2, 2.3 Hz, 1H), 7.91 (d, J=1.4 Hz, 1H), 7.82 (d, J=8.3 Hz, 2H), 7.46 (d, J=8.3 Hz, 2H), 5.67 (s, 2H), 3.85 (s, 3H), 3.69 (tt, J=7.7, 4.0 Hz, 1H), 1.77 (tt, J=8.3, 4.7 Hz, 1H), 1.04 (p, J=3.5 Hz, 2H), 0.95 (dd, J=7.5, 5.0 Hz, 2H), (d, J=4.5 Hz, 2H), 0.79 (dq, J=6.9, 3.4 Hz, 2H).

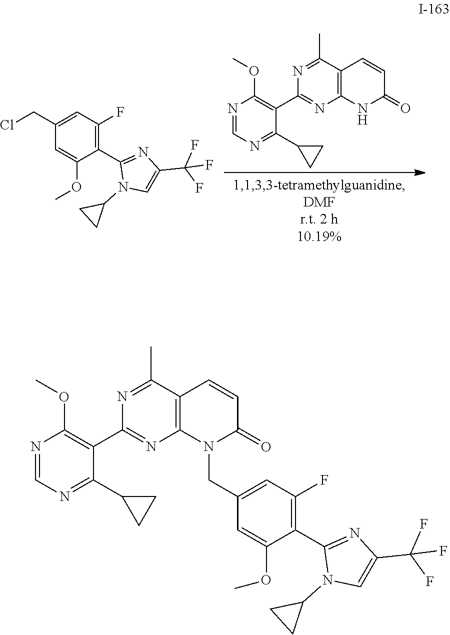

To a stirred solution of 2-[4-(chloromethyl)-2-fluoro-6-methoxyphenyl]-1-cyclopropyl-4-(trifluoromethyl)imidazole (112 mg, 0.321 mmol, 1 equiv) and 2-(4-cyclopropyl-6-methoxypyrimidin-5-yl)-4-methyl-8H-pyrido[2,3-d]pyrimidin-7-one (99.3 mg, 0.321 mmol, 1 equiv) in DMF (2 mL, 25.843 mmol, 80.47 equiv) were added 1,1,3,3-tetramethylguanidine (110.9 mg, 0.963 mmol, 3 equiv) at room temperature under nitrogen atmosphere. The resulting mixture was stirred for additional 2 h at room temperature. The resulting mixture was filtered, the filter cake was washed with MeCN (3×5 mL), filtered and concentrated under reduced pressure. The crude product (100 mg) was purified by Prep-HPLC with the following conditions (Column: Sunfire prep C18 column, 30*150 mm, 51 μm; Mobile Phase A: Water(0.1% FA), Mobile Phase B: MeOH-HPLC; Flow rate: 60 mL/min; Gradient: 53% B to 73% B in 10 min; Wave Length: 254 nm/220 nm; RT1(min): 12.22) to afford 8-({4-[1-cyclopropyl-4-(trifluoromethyl)imidazo]-2-yl]-3-fluoro-5-methoxyphenyl}methyl)-2-(4-cyclopropyl-6-methoxypyrimidin-5-yl)-4-methylpyrido[2,3-d]pyrimidin-7-one (21.2 mg, 10.19%) as an off-white solid.

LC-MS (ESI) m/z 622[M+H]

¹H NMR (400 MHz, DMSO-d6) δ 8.68 (s, 1H), 8.32 (d, J=9.7 Hz, 1H), 7.91 (s, 1H), 7.04 (s, 1H), 6.88 (d, J=9.8 Hz, 1H), 6.68 (d, J=9.9 Hz, 1H), 5.57 (d, J=7.2 Hz, 2H), 3.81 (s, 3H), 3.71 (s, 3H), 3.18 (d, J=5.2 Hz, 1H), 2.82 (s, 3H), 1.76-1.69 (m, 1H), 1.01 (m, 2H), (m, 1H), 0.80 (d, J=8.2 Hz, 3H), 0.69 (d, J=8.0 Hz, 2H).

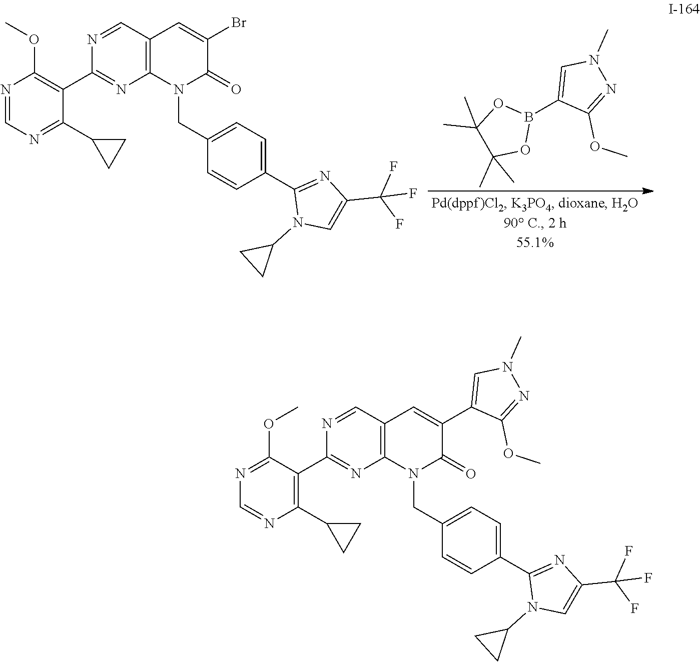

To a stirred mixture of 6-bromo-8-({4-(1-cyclopropyl-4-yl)pyrido[2,3-d]pyrimidin-7-one (80 mg, 0.125 mmol, 1 equiv) and 3-methoxy-1-methyl-4-(4,4,5,5-tetramethyl-1,3,2-dioxaborolan-2-yl)pyrazole (44.7 mg, 0.188 mmol, 1.5 equiv) in H₂O (0.3 mL) and dioxane (1.2 mL) were added K₃PO₄ (66.5 mg, 0.313 mmol, 2.5 equiv) and Pd(dppf)C12 (18.3 mg, 0.025 mmol, 0.2 equiv) at room temperature under nitrogen atmosphere. The resulting mixture was stirred for 2 h at under nitrogen atmosphere. The mixture was allowed to cool down to room temperature. The resulting mixture was diluted with water (20 mL). The resulting mixture was extracted with EtOAc (3×20 mL). The combined organic layers were washed with brine (3×20 mL), dried over anhydrous Na₂SO₄, filtered and concentrated under reduced pressure. The crude product (100 mg) was purified by Prep-HPLC with the following conditions (Column: XBridge Shield RP18 OBD Column 30*150 mm, 5 μm; Mobile Phase A: Water(10 mmol/L NH₄HCO₃), Mobile Phase B: ACN; Flow rate: 60 mL/min; Gradient: 41% B to 61% B in 10 min; Wave Length: 254 nm/200 nm; RT1(min): 11.22) to afford 8-({4-[1-cyclopropyl-4-(trifluoromethyl)imidazo]-2-yl]phenyl}methyl)-2-(4-cyclopropyl-6-methoxypyrimidin-5-yl)-6-(3-methoxy-1-methylpyrazol-4-yl)pyrido[2,3-d]pyrimidin-7-one (46.8 mg, 55.16%) as an off-white solid.

LC-MS (ESI) m/z 670.2 [M+H]

¹H NMR (400 MHz, DMSO-d6) δ 9.34 (s, 1H), 8.69 (s, 1H), 8.50 (d, J=12.1 Hz, 2H), 7.91 (s, 1H), 7.81 (d, J=8.1 Hz, 2H), 7.41 (d, J=8.1 Hz, 2H), 5.68 (s, 2H), 4.01 (s, 3H), 3.84 (s, 3H), 3.79-3.75 (m, 3H), 3.69 (d, J=4.3 Hz, 1H), 1.76 (dt, J=8.4, 4.8 Hz, 1H), 1.02 (t, J=3.8 Hz, 2H), 0.96 (q, J=6.4, 5.2 Hz, 2H), 0.90 (s, 2H), 0.78 (dd, J=7.9, 3.5 Hz, 2H).

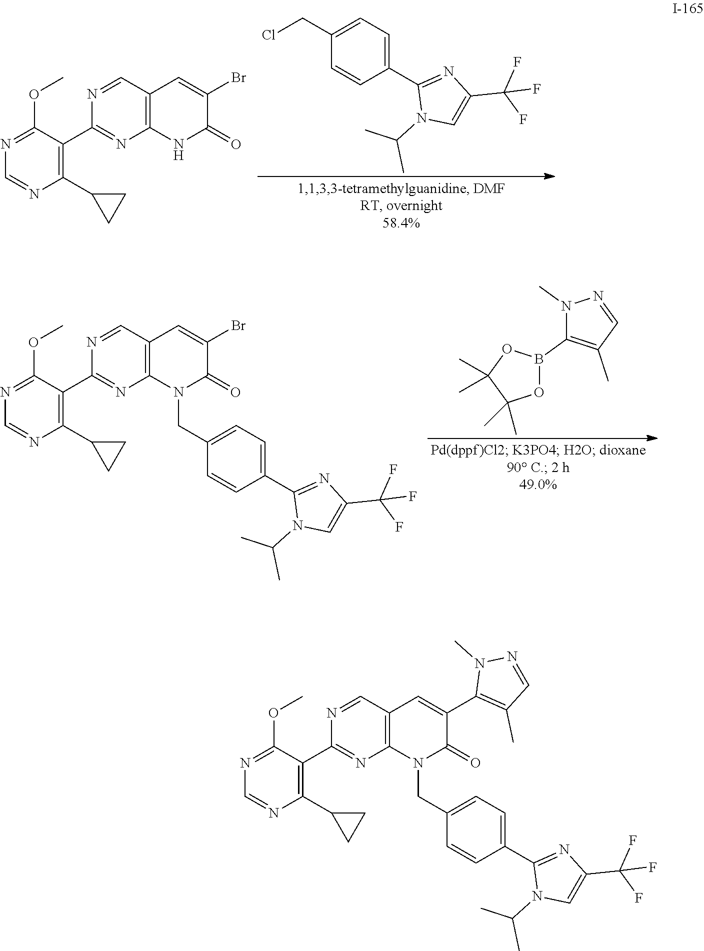 [SMILES: COc1ncnc(C2CC2)c1-c1ncc2cc(Br)c(=O)[nH]c2n1 ; CC(C)n1cc(C(F)(F)F)nc1-c1ccc(CCl)cc1 ; COc1ncnc(C2CC2)c1-c1ncc2cc(Br)c(=O)n(Cc3ccc(-c4nc(C(F)(F)F)cn4C(C)C)cc3)c2n1 ; Cc1cnn(C)c1B1OC(C)(C)C(C)(C)O1 ; COc1ncnc(C2CC2)c1-c1ncc2cc(-c3c(C)cnn3C)c(=O)n(Cc3ccc(-c4nc(C(F)(F)F)cn4C(C)C)cc3)c2n1]

### Step 1: 6-Bromo-2-(4-Cyclopropyl-6-Methoxypyrimidin-5-Yl)-8-({4-[1-Isopropyl-4-(Trifluoromethyl)Imidazol-2-Yl]Phenyl}Methyl)Pyrido[2,3-d]Pyrimidin-7-One

To a stirred solution of 6-bromo-2-(4-cyclopropyl-6-methoxypyrimidin-5-yl)-8H-pyrido[2,3-d]pyrimidin-7-one (100 mg, 0.267 mmol, 1 equiv) and 2-[4-(chloromethyl)phenyl]-1-isopropyl-4-(trifluoromethyl)imidazole (80.90 mg, 0.267 mmol, 1 equiv) in DMF (2 mL) was added 1,1,3,3-tetramethylguanidine (61.56 mg, 0.534 mmol, 2 equiv) at room temperature under nitrogen atmosphere. The resulting mixture was stirred for overnight at room temperature under nitrogen atmosphere. The residue was purified by reverse flash chromatography (C18 silica gel column, eluting with 10% to 50% MeCN/water (0.1% FA) in 10 min with UV detector (254 nm)) to afford 6-bromo-2-(4-cyclopropyl-6-methoxypyrimidin-5-yl)-8-({4-[1-isopropyl-4-(trifluoromethyl)imidazol-2-yl]phenyl}methyl)pyri do[2,3-d]pyrimidin-7-one (100 mg, 58.43%) as an white solid.

LC-MS (ESI) m/z 641.4 [M+H]

### Step 2: 2-(4-Cyclopropyl-6-Methoxypyrimidin-5-Yl)-6-(2,4-Dimethylpyrazol-3-Yl)-8-({4-[1-Isopropyl-4-(Trifluoromethyl)Imidazol-2-Yl]Phenyl}Methyl)Pyrido[2,3-d]Pyrimidin-7-One

To a stirred solution of 6-bromo-2-(4-cyclopropyl-6-methoxypyrimidin-5-yl)-8-({4-[1-isopropyl-4-(trifluoromethyl)imidazol-2-yl]phenyl}methyl)pyri do[2,3-d]pyrimidin-7-one (50 mg, 0.078 mmol, 1 equiv) and 1,4-dimethyl-5-(4,4,5,5-tetramethyl-1,3,2-dioxaborolan-2-yl)pyrazole (26.01 mg, 0.117 mmol, 1.5 equiv) in dioxane (1 mL) and H₂O (0.25 mL) were added Pd(dppf)Cl₂ (11.42 mg, 0.016 mmol, 0.2 equiv) and K₃PO₄ (41.43 mg, 0.195 mmol, 2.5 equiv) at room temperature under nitrogen atmosphere. The resulting mixture was stirred for 2h at under nitrogen atmosphere. The mixture was allowed to cool down to room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The crude product (50 mg) was purified by Prep-HPLC with the following conditions (Column: XBridge Shield RP18 OBD Column 30*150 mm, 51 μm; Mobile Phase A: Water(10 mmol/L NH₄HCO₃), Mobile Phase B: ACN; Flow rate: 60 mL/min; Gradient: 36% B to 56% B in 10 min; Wave Length: 254 nm/220 nm; RT1(min): 10.98) to afford 2-(4-cyclopropyl-6-methoxypyrimidin-5-yl)-6-(2,4-dimethylpyrazol-3-yl)-8-({4-[1-isopropyl-4-(trifluoromethyl)imidazol-2-yl]phenyl}methyl)pyrido[2,3-d]pyrimidin-7-one (25.1 mg, 49.03%) as an off-white solid.

LC-MS (ESI) m/z 655.6 [M+H]

¹H NMR (400 MHz, DMSO-d6) δ 9.35 (s, 1H), 8.70 (s, 1H), 8.29 (s, 1H), 8.15 (d, J=1.5 Hz, 1H), 7.53-7.43 (m, 4H), 7.36 (s, 1H), 5.66 (s, 2H), 4.42 (p, J=6.6 Hz, 1H), 3.84 (s, 3H), 3.69 (s, 3H), 2.00 (s, 3H), 1.75 (tt, J=8.3, 4.6 Hz, 1H), 1.38 (d, J=6.6 Hz, 6H), 1.03 (p, J=3.8 Hz, 2H), 0.76 (dq, J=6.9, 3.5 Hz, 2H).

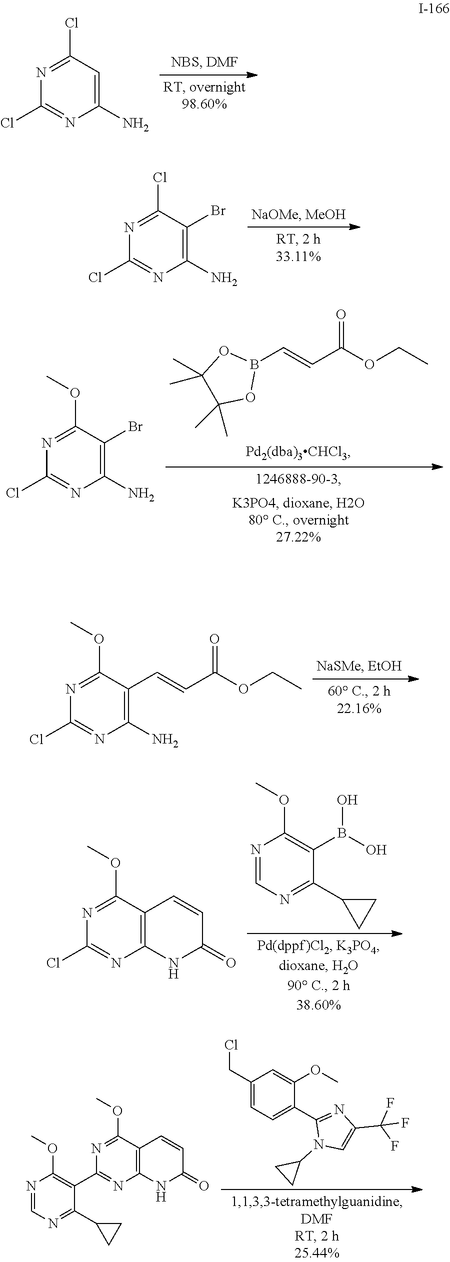

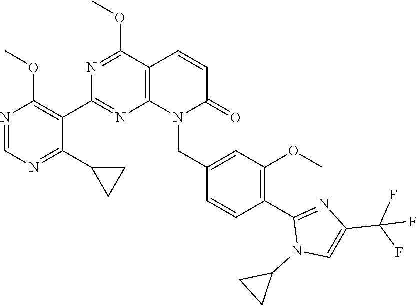 [SMILES: COc1cc(Cn2c(=O)ccc3c(OC)nc(-c4c(OC)ncnc4C4CC4)nc32)ccc1-c1nc(C(F)(F)F)cn1C1CC1]

### Step 1: 5-Bromo-2,6-Dichloropyrimidin-4-Amine

To a stirred solution of 2,6-dichloropyrimidin-4-amine (3.15 g, 19.208 mmol, 1 equiv) in DMF (30 mL) was added NB S (3.76 g, 21.129 mmol, 1.10 equiv) at room temperature. The resulting mixture was stirred for overnight at room temperature. The resulting mixture was diluted with water (60 mL). The resulting mixture was extracted with EtOAc (2×40 mL). The combined organic layers were washed with brine (2×60 mL), dried over anhydrous Na₂SO₄, filtered and concentrated under reduced pressure to afford 5-bromo-2,6-dichloropyrimidin-4-amine (4.6 g, 98.60%) as a light yellow solid.

LC-MS (ESI) m/z 243.8 [M+H]

### Step 2: 5-Bromo-2-Chloro-6-Methoxypyrimidin-4-Amine

To a stirred solution of 5-bromo-2,6-dichloropyrimidin-4-amine (4.74 g, 19.515 mmol, 1 equiv) in MeOH (80 mL) was added NaOMe (2.81 g, 15.612 mmol, 0.8 equiv, 30% in MeOH) at room temperature. The resulting mixture was stirred for 2 h at room temperature. The resulting mixture was concentrated under reduced pressure. The resulting mixture was diluted with water (60 mL). The resulting mixture was extracted with EtOAc (2×50 mL). The combined organic layers were washed with brine (2×50 mL), dried over anhydrous Na₂SO₄, filtered and concentrated under reduced pressure. The residue was purified by silica gel column chromatography, (eluting with PE/EA (10:1)) to afford 5-bromo-2-chloro-6-methoxypyrimidin-4-amine (1.541 g, 33.11%) as an off-white solid.

LC-MS (ESI) m/z 239.4 [M+H]

¹H NMR: (400 MHz, DMSO-d6) δ 8.12-6.60 (m, 2H), 3.90 (s, 3H).

### Step 3: Ethyl (2E)-3-(4-Amino-2-Chloro-6-Methoxypyrimidin-5-Yl)Prop-2-Enoate

To a stirred solution of 5-bromo-2-chloro-6-methoxypyrimidin-4-amine (1.7 g, 7.129 mmol, 1 equiv) and ethyl (2E)-3-(4,4,5,5-tetramethyl-1,3,2-dioxaborolan-2-yl)prop-2-enoate (94.80 mg, 0.419 mmol, 1 equiv) in dioxane (20 mL) and H₂O (4 mL) were added K₃PO₄ (3.03 g, 14.258 mmol, 2 equiv), 3-tert-butyl-4-(2,6-dimethoxyphenyl)-2,3-dihydro-1,3-benzoxaphosphole (170 mg, 0.515 mmol, 0.07 equiv) and Pd 2 (dba) 3.CHCl₃ (170 mg, 0.164 mmol, equiv) at room temperature. The resulting mixture was stirred for overnight at 80° C. under nitrogen atmosphere. The mixture was allowed to cool down to room temperature. The resulting mixture was diluted with water (40 mL). The resulting mixture was extracted with EtOAc (2×30 mL). The combined organic layers were washed with brine (2×30 mL), dried over anhydrous Na₂SO₄, filtered and concentrated under reduced pressure. The residue was purified by reversed-phase flash chromatography (C18 silica gel column, eluting with 0% to 100% MeCN/water (0.1% FA) in 20 min with UV detector (254 nm)) to afford ethyl (2E)-3-(4-amino-2-chloro-6-methoxypyrimidin-5-yl)prop-2-enoate (500 mg, 27.22%) as a light yellow solid.

LC-MS (ESI) m/z 258.0 [M+H]

### Step 4: 2-Chloro-4-Methoxy-811-Pyrido[2,3-d]Pyrimidin-7-One

To a stirred solution of ethyl (2E)-3-(4-amino-2-chloro-6-methoxypyrimidin-5-yl)prop-2-enoate (500 mg, 1.940 mmol, 1 equiv) in EtOH (10 mL) was added Sodiummethanethiolate (122.4 mg, 1.747 mmol, 0.90 equiv) at room temperature. The resulting mixture was stirred for 2 h at 60° C. The mixture was allowed to cool down to room temperature. The mixture was purified by reversed-phase flash chromatography (C18 silica gel column, eluting with 0% to 100% MeCN/water (0.1% FA) in 10 min with UV detector (254 nm)) to afford 2-chloro-4-methoxy-8H-pyrido[2,3-d]pyrimidin-7-one (91 mg, 22.16%) as a light yellow solid.

LC-MS (ESI) m/z 212.0 [M+H]

### Step 5: 2-(4-Cyclopropyl-6-Methoxypyrimidin-5-Yl)-4-Methoxy-811-Pyrido[2,3-d]Pyrimidin-7-One

To a stirred solution of 2-chloro-4-methoxy-8H-pyrido[2,3-d]pyrimidin-7-one (91 mg, 0.430 mmol, 1 equiv) and 4-cyclopropyl-6-methoxypyrimidin-5-ylboronic acid (125.1 mg, mmol, 1.50 equiv) in dioxane (2 mL) and H₂O (1 mL) were added K₃PO₄ (228.2 mg, 1.075 mmol, 2.50 equiv) and Pd(dppf)C12 (62.9 mg, 0.086 mmol, 0.20 equiv) at room temperature. The resulting mixture was stirred for 2 h at 90° C. under nitrogen atmosphere. The mixture was allowed to cool down to room temperature. The resulting mixture was filtered; the filter cake was washed with THF (2×5 mL). The filtrate was concentrated under reduced pressure. The residue was purified by reversed-phase flash chromatography (C18 silica gel column, eluting with 0% to 100% MeCN/water (0.1% FA) in 10 min with UV detector (254 nm)) to afford 2-(4-cyclopropyl-6-methoxypyrimidin-5-yl)-4-methoxy-8H-pyrido[2,3-d]pyrimidin-7-one (54 mg, 38.60%) as a brown solid.

LC-MS (ESI) m/z 326.1 [M+H]

### Step 6: 8-({4-[1-Cyclopropyl-4-(Trifluoromethyl)Imidazol-2-Yl]-3-Methoxyphenyl}Methyl)-2-(4-Cyclopropyl-6-Methoxypyrimidin-5-Yl)-4-Methoxypyrido[2,3-d]Pyrimidin-7-One

To a stirred solution of 2-(4-cyclopropyl-6-methoxypyrimidin-5-yl)-4-methoxy-8H-pyrido[2,3-d]pyrimidin-7-one (58 mg, 0.178 mmol, 1 equiv) and 2-[4-(chloromethyl)-2-methoxyphenyl]-1-cyclopropyl-4-(trifluoromethyl)imidazole (59 mg, 0.178 mmol, 1.00 equiv) in DMF (2 mL) was added 1,1,3,3-tetramethylguanidine (30.80 mg, 0.267 mmol, 1.5 equiv) at room temperature. The resulting mixture was stirred for 2 h at room temperature. The mixture was purified by reversed-phase flash chromatography (C18 silica gel column, eluting with 0% to 100% MeCN/water (0.1% FA) in 10 min with UV detector (254 nm)) to afford the crude product. The crude product (60 mg) was purified by Prep-HPLC with the following conditions (Column: Sunfire prep C18 column, 30*150 mm, 51 μm; Mobile Phase A: Water(0.1% FA), Mobile Phase B: MeOH-HPLC; Flow rate: 60 mL/min; Gradient: 53% B to 73% B in 10 min; Wave Length: 254 nm/220 nm; RT1(min): 10) to afford 8-({4-[1-cyclopropyl-4-(trifluoromethyl)imidazo]-2-yl]-3-methoxyphenyl methyl)-2-(4-cyclopropyl-6-methoxypyrimidin-5-yl)-4-methoxypyrido[2,3-d]pyrimidin-7-one (28.1 mg, 25.44%) as an off-white solid.

LC-MS (ESI) m/z 620.2 [M+H]

¹H NMR: (400 MHz, DMSO-d6) δ 8.67 (s, 1H), 8.10 (d, J=9.6, 1.7 Hz, 1H), 7.83 (s, 1H), 7.22 (d, J=7.8, 1.7 Hz, 1H), 7.15 (s, 1H), 6.83-6.74 (m, 2H), 5.57 (s, 2H), 4.09 (s, 3H), 3.84 (s, 3H), 3.70 (s, 3H), 3.30-3.26 (m, 1H), 1.89 (dt, J=8.0, 3.7 Hz, 1H), 1.24 (s, OH), 1.05-(m, 2H), 0.81 (dq, J=7.3, 3.4 Hz, 2H), 0.76-0.70 (m, 4H).

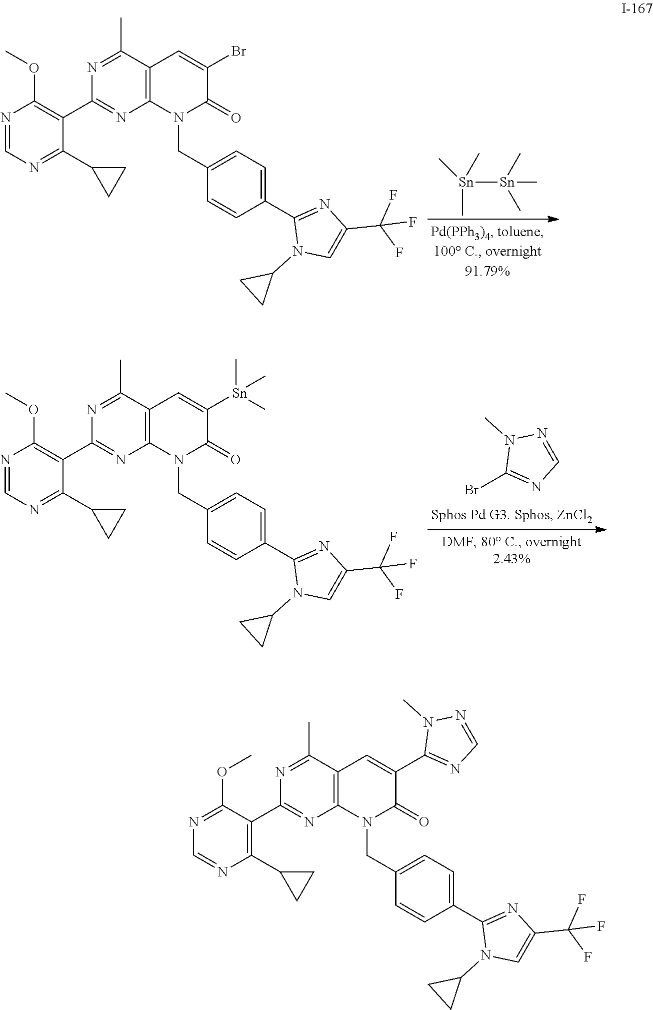

### Step 1: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-4-Methyl-6-(Trimethylstannyl)Pyrido[2,3-d]Pyrimidin-7-One

To a stirred solution of 6-bromo-8-({4-[1-cyclopropyl-4-(trifluoromethyl)imidazo]-2-yl]phenyl}methyl)-2-(4-cyclopropyl-6-methoxypyrimidin-5-yl)-4-methylpyrido[2,3-d]pyrimidin-7-one (125 mg, 0.192 mmol, 1 equiv) and hexamethyldistannane (125.5 mg, 0.384 mmol, 2 equiv) in Toluene (2 mL, 18.797 mmol, 98.12 equiv) were added Pd(PPh₃) 4 (11.1 mg, 0.010 mmol, 0.05 equiv) at room temperature under nitrogen atmosphere. The resulting mixture was stirred for additional overnight at 100° C. The mixture was allowed to cool down to room temperature. The precipitated solids were collected by filtration and washed with MeCN (3×3 mL). The residue was purified by reverse flash chromatography (C18 silica gel column, eluting with 0% to 100% MeCN/water (0.1% FA) in 10 min with UV detector (254 nm)) to afford 8-({4-[1-cyclopropyl-4-(trifluoromethyl)imidazo]-2-yl]phenyl}methyl)-2-(4-cyclopropyl-6-methoxypyrimidin-5-yl)-4-methyl-6-(trimethyl stannyl)pyrido[2,3-d]pyrimidin-7-one (129.5 mg, 91.79%) as a colorless solid.

LC-MS (ESI) m/z 738[M+H]

### Step 2: 8-({4-[1-Cyclopropyl-4-(Trifluoromethyl)Imidazol-2-Yl]Phenyl}Methyl)-2-(4-Cyclopropyl-6-Methoxypyrimidin-5-Yl)-4-Methyl-6-(2-Methyl-1,2,4-Triazol-3-Yl)Pyrido[2,3-d]Pyrimidin-7-One

To a stirred solution of 8-({4-[1-cyclopropyl-4-(trifluoromethyl)imidazo]-2-yl]phenyl}methyl)-2-(4-cyclopropyl-6-methoxypyrimidin-5-yl)-4-methyl-6-(trimethylstannyl)pyrido[2,3-d]pyrimidin-7-one (129.5 mg, 0.176 mmol, 1 equiv) and 5-bromo-1-methyl-1,2,4-triazole (56.97 mg, 0.352 mmol, 2 equiv) in DMF (3 mL, 38.765 mmol, 220.43 equiv) were added Sphos (13 mg, 0.032 mmol, 0.18 equiv) and SPhos Pd G3 (13 mg, 0.017 mmol, equiv) at room temperature under nitrogen atmosphere. To the above mixture was added ZnCl₂ (23.97 mg, 0.176 mmol, 1 equiv) at room temperature. The resulting mixture was stirred for additional overnight at 80° C. The mixture was allowed to cool down to room temperature. The precipitated solids were collected by filtration and washed with MeCN (3×3 mL). The crude product (100 mg) was purified by Prep-HPLC with the following conditions (Column: Xselect CSH C18 OBD Column 30*150 mm 51 μm; Mobile Phase A: Water(0.1% FA), Mobile Phase B: ACN; Flow rate: 60 mL/min; Gradient: 35% B to 55% B in 8 min; Wave Length: 254 nm/220 nm; RT1(min): 7.8) to afford 8-({4-[1-cyclopropyl-4-(trifluoromethyl)imidazo]-2-yl]phenyl}methyl)-2-(4-cyclopropyl-6-methoxypyrimidin-5-yl)-4-methyl-6-(2-methyl-1,2,4-triazol-3-yl)pyrido[2,3-d]pyrimidin-7-one (2.8 mg, 2.43%) as an off-white solid.

LC-MS (ESI) m/z 655[M+H]

¹H NMR (400 MHz, DMSO-d6) δ 8.70 (s, 1H), 8.63 (s, 1H), 8.08 (s, 1H), 7.90 (d, J=1.4 Hz, 1H), 7.81 (d, J=8.3 Hz, 2H), 7.44 (d, J=8.2 Hz, 2H), 5.67 (s, 2H), 3.84 (s, 3H), 3.81 (s, 3H), 3.70 (td, J=7.1, 3.5 Hz, 1H), 2.87 (s, 3H), 1.74 (dt, J=8.2, 3.8 Hz, 1H), 1.06-1.00 (m, 2H), 0.99-0.93 (m, 2H), 0.89 (m, 2H), 0.78 (dd, J=7.9, 3.3 Hz, 2H).

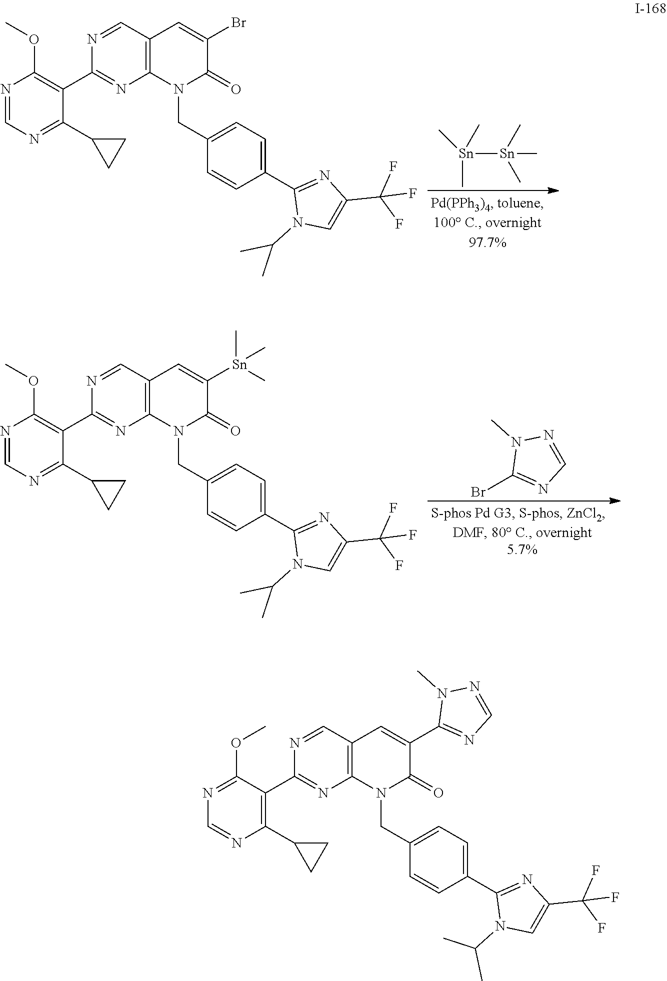

### Step 1: 2-(4-Cyclopropyl-6-Methoxypyrimidin-5-Yl)-8-({4-[1-Isopropyl-4-(Trifluoromethyl)Imidazol-2-Yl]Phenyl}Methyl)-6-(Trimethylstannyl)Pyrido[2,3-d]Pyrimidin-7-One

To a stirred solution of 6-bromo-2-(4-cyclopropyl-6-methoxypyrimidin-5-yl)-8-({4-[1-isopropyl-4-(trifluoromethyl)imidazol-2-yl]phenyl}methyl)pyri do[2,3-d]pyrimidin-7-one (200 mg, 0.312 mmol, 1 equiv) and hexamethyldistannane (204.62 mg, 0.624 mmol, 2 equiv) in toluene (3 mL) was added Pd(PPh₃)₄ (18.04 mg, 0.016 mmol, 0.05 equiv) in portions at room temperature under nitrogen atmosphere. The resulting mixture was stirred for overnight at 100° C. under nitrogen atmosphere. The mixture was allowed to cool down to room temperature. The resulting mixture was diluted with water (100 mL). The resulting mixture was extracted with EtOAc (3×10 mL). The combined organic layers were washed with brine (3×20 mL), dried over anhydrous Na₂SO₄. After filtration, the filtrate was concentrated under reduced pressure. The residue was purified by reverse flash chromatography (C18 silica gel column, eluting with 0% to 80% MeCN/water (0.1% FA) in 10 min with UV detector (254 nm)) to afford 2-(4-cyclopropyl-6-methoxypyrimidin-5-yl)-8-({4-[1-isopropyl-4-(trifluoromethyl)imidazol-2-yl]phenyl}methyl)-6-(trimethylstannyl)pyrido[2,3-d]pyrimidin-7-one (220 mg, 97.26%) as a white solid.

LC-MS (ESI) m/z 725.3 [M+H]

### Step 2: 2-(4-Cyclopropyl-6-Methoxypyrimidin-5-Yl)-8-({4-[1-Isopropyl-4-(Trifluoromethyl)Imidazol-2-Yl]Phenyl}Methyl)-6-(2-Methyl-1,2,4-Triazol-3-Yl)Pyrido[2,3-d]Pyrimidin-7-One

To a stirred mixture of 2-(4-cyclopropyl-6-methoxypyrimidin-5-yl)-8-({4-[1-isopropyl-4-(trifluoromethyl)imidazo]-2-yl]phenyl}methyl)-6-(trimethylstannyl)pyrido[2,3-d]pyrimidin-7-one (170 mg, 0.235 mmol, 1 equiv) and 5-bromo-1-methyl-1,2,4-triazole (76.03 mg, 0.470 mmol, 2 equiv) in DMF (2 mL) were added Xphos Pd G3 (17 mg, 0.020 mmol, 0.09 equiv), Xphos (17 mg, 0.036 mmol, 0.15 equiv) and ZnCl₂ (31.98 mg, 0.235 mmol, 1 equiv) at room temperature under nitrogen atmosphere. The resulting mixture was stirred for overnight at 80° C. under nitrogen atmosphere. The mixture was allowed to cool down to room temperature. The resulting mixture was filtered; the filter cake was washed with THF (3×2 mL). The filtrate was concentrated under reduced pressure. The crude product (170 mg) was purified by Prep-HPLC with the following conditions (Column: YMC-Actus Triart C18 ExRS 30*150 mm, 5 μm; Mobile Phase A: Water(10 mmol/L NH₄HCO₃+0.05%NH₃H₂O), Mobile Phase B: ACN; Flow rate: 60 mL/min; Gradient: 40% B to 57% B in 10 min; Wave Length: 254 nm/220 nm; RT1(min): 9.95) to afford 2-(4-cyclopropyl-6-methoxypyrimidin-5-yl)-8-({4-[1-isopropyl-4-(trifluoromethyl)imidazo]-2-yl]phenyl}methyl)-6-(2-methyl-1,2,4-triazol-3-yl)pyrido[2,3-d]pyrimidin-7-one (8.7 mg, 5.77%) as an off-white solid.

LC-MS (ESI) m/z 643.6 [M+H]

¹H NMR (400 MHz, DMSO-d6) δ 9.43 (s, 1H), 8.71 (s, 1H), 8.57 (s, 1H), 8.20-8.12 (m, 1H), 8.08 (s, 1H), 7.48 (s, 4H), 5.67 (s, 2H), 4.41 (p, J=6.6 Hz, 1H), 3.83 (d, J=9.9 Hz, 6H), 1.75 (dt, J=8.2, 4.6 Hz, 1H), 1.37 (d, J=6.6 Hz, 6H), 1.03 (d, J=4.1 Hz, 2H), 0.84-0.67 (m, 2H).

### Example 2. Biological Assays

### USP1 Western Blot Protocol (HCC1806 Ub-PCNA)

USP1 is a deubiquitinase that catalyze the hydrolysis of iso-peptide bond between ubiquitin and the substrate proteins. Inhibition of USP1 activity results in accumulation of mono-ubiquitinated substrate such as Ub-PCNA. A cell assay was set up to determine the cellular potency of USP1 compounds by monitoring the accumulation of Ub-PCNA. The signal intensity was detected by a specific Ub-PCNA antibody through Western-blot analysis.

The assay was initiated by seeding HCC1806 cells (ATCC, CRL-2335) into 6-well plates (Corning, 3516), with a seeding density of 3×10⁵ cells per well and 2 mL RPMI 1640 culture medium (Gibco, 11875119). The plates were incubated overnight at 37° C. and 5% CO₂. Compounds, solubilized in DMSO (Solarbio, D8371) as 1 mM stock solution, were diluted in 4-fold steps for 5-cencentration point dose response plus a no-compound DMSO control. Diluted compounds (2 μL) were transferred into assay wells (with a final DMSO concentration of −0.1%) and the resulted assay plates incubated at 37° C. and 5% CO₂ for 4 hours. After aspiration of culture medium, 100 μL of 4° C. RIPA lysis buffer (Boston BioProducts, BP-115D) supplemented with vendor recommended concentrations of protease (Roche, 4693116001) and phosphatase (Roche, 4906837001) inhibitor, was added to each well and incubated for 10 minutes on the ice. Cells were scrapped loose from the bottom of well with a scraper (Corning, 3008), transferred into a 600 μL EP tube, sonicated using Ultrasound (Qsonica, Q800R3, with 30% energy, 5-second pulse on/off cycles at 4° C., for total 2 minutes), centrifuged 13,000 rpm for 15 minutes at 4° C., and then the supernatant transferred to a new 600 μL EP tube. The total protein concentration was determined using BCA kit following vendor recommended protocol (Solarbio, PC0020-500). Loading samples were prepared by diluting the total protein concentration to 1.5 μg/μL with 5 X NuPAGE SDS loading buffer (Beyotime, P0015L) and RIPA lysis buffer, and boiling at 100° C. for 5 minutes.

15 μg protein from each sample was loaded onto NuPAGE® Novex 4-12% Bis-Tris Midi Gel, 26 W (Invitrogen, WG1403BOX) and electrophoresis was run at 80 V for 30 minutes and 120 V till the end using 1x NuPAGE® MOPS SDS Running Buffer (Novex, NP0001). The gel was then transferred to nitrocellulose membrane using the dry transfer system (iBlot 2 Transfer Stacks, nitrocellulose, D323001) with a program consisting of 20 V for 1 minute, 23 V for 4 minutes and 25 V for 2 minutes. After the membrane was blocked with 5% BSA (Solarbio, A8020) in 1X TBST (CST, 9997S) for 1 hour at room temperature, specific antibodies (Ubiquityl-PCNA antibody (CST, 13439) at 1:500 dilution, PCNA antibody (Santa Cruz, sc-56) at 1:1000 dilution, and GAPDH (CST, 5174) at 1:2000 dilution in 1 X TBST containing 1% BSA) were added and incubated at room temperature for 1.5 hours. After washing the Nitrocellulose membranes with 1 X TBST for 3 times, 10 minutes each time at room temperature, fluorescently labeled secondary antibodies (anti-rabbit IgG, Licor, 926-32211; anti-mouse IgG, LI-COR, 926-68070) at 1:5000 dilution in 1 X TBST containing 1% BSA were added and incubated for 1 hour at room temperature in the dark. The membranes were then rinsed with 1 X TBST and immersed in the RO water. Imaging of band intensity was acquired with Li—COR imaging system (Li—COR, Odyssey clx ES).

Percentage of USP1 inhibition (% Vehicle) was calculated by using following equation examining Ub-PCNA upregulation:

$$\% Vehicle = \frac{Grey value of compound}{Grey value of DMSO} * 100 \%$$

And IC₅₀ was calculated using four-parameter logistic fitting program
Y=Bottom+(Top-Bottom)/(1+10 {circumflex over (()}(LogIC50−X)*Hill Slope))
X=Log of dose or concentration

### CTG Protocol (MDA-MB-436 GI50)

Antiproliferative activity of USP1 inhibitors was determined using breast cancer cell line MDA-MB-436 that harbors BRCA1 mutation. MDA-MB-436 cells (ATCC, HTB-130) was seeded with a seeding density of 1000 cells per well and 95 μL DMEM medium (Gibco, 10566016) into 96-well cell plates (Corning, 3610), and incubated overnight at 37° C. and 5% CO₂. Compounds were solubilized in DMSO (Solarbio, D8371) as 10 mM stock solution, and then series diluted (3 folds) with DMSO for 9-point dose-response testing. The resulted series dilutions were then jump-diluted 50-fold by cell culture medium to afford the 20×stock solutions (containing 2% DMSO). Treatment was initiated by adding 5 μL of 20×stock solution to the assay plate and incubated at 37° C. and 5% CO₂. At the end of 7-day incubation, cell assay plates were removed from incubation and equilibrated to room temperature (20 min). Then 100 μL of CellTiter-Glo (Promega, G9243) reagent, prepared per manufacturer's protocol, was added to each well, and the resulted plates were centrifuged 1,000 rpm for 1 minute, incubated at room temperature in the dark for 20 minutes, sealed with Perkin Elmer sticky film, and the luminescence signal read by Envision (Perkin Elmer).

Data analysis
a) % Inhibition was calculated using following equation:

$$\% Inhibition = 100 \% \times \frac{{Signal}_{Average ( HC )} - {Signal}_{sample}}{{Signal}_{Average ( HC )} - {Signal}_{Average ( LC )}}$$

HC: DMSO; LC: 1 μM of a known USP1 inhibitor as Formula I from WO2021163530.
b) IC₅₀ was calculated by fitting data to following equation:

$$Y = Bottom + \frac{Top - Bottom}{1 + {( IC 50 / X )}^{slope}}$$

X: compound concentration; Y: % Inhibition

### Enzyme Assay

Enzymatic inhibitory potency of the compounds was assessed in a deubiquitinase fluorometric assay using ubiquitin-Rhodamine (Ub-Rho110, R&D Systems) as substrate and the recombinant USP1/UAF1 enzyme complex (Biortus). Assays were performed in a final volume of 12 μL using black 384-well plates (Corning #4514) containing 50 mM HEPES (pH 7.5, BioRoYee), 0.5 mM EDTA, 1 mM dithiothreitol, 0.1 mg/mL bovine serum albumin, 50 nM Ub-Rhol10, 0.004 nM USP1/UAF1, and series diluted compounds from 10 μM to 0.5 nM (10 dilution steps). Assay-ready plates containing 40 nL of Echo-dispensed compound in DMSO were reconstituted with 2 μL of ddH₂O, then 5 μL of USP1/UAF1 enzyme mixture was added and preincubated at room temperature for 2 hours. Enzymatic reaction was initiated by the addition of μL of Ub-Rhol10 substrate mixture and assay allowed to proceed for 1 hour at room temperature before quenching with 3 μL of 150 mM citric acid. Fluorescence signal was detected via TECAN SPARK with excitation at 485 nm and emission at 535 nm. Data analysis was done using GraphPad Prism8 and IC₅₀ values were obtained by 4-parameter logistic curve fitting.

Certain compounds were tested in the assays. The data are listed in Table 2 below. A: IC_{50<10} nM; B: 10 nM<IC50<100 nM; C: 100 nM<IC50<1000 nM; D: IC50 >1000 nM.

TABLE 2
In vitro Data of Certain Exemplary Compounds.

| | | HCC1806 | MDA-MB-436 |
|---|---|---|---|
| I# | enzyme (nM) | Ub-PCNA (nM) | GI50 (nM) |
| 1 | B | A | A |
| 2 | B | B | B |
| 3 | B | B | B |
| 4 | B | B | A |
| 5 | B | B | C |
| 6 | B | B | B |
| 7 | B | B | B |
| 8 | B | B | B |
| 9 | B | B | B |
| 10 | B | B | B |
| 11 | B | B | B |
| 12 | B | C | C |
| 13 | C | C | C |
| 14 | B | B | A |
| 15 | A | B | A |
| 16 | B | C | B |
| 17 | B | B | B |
| 18 | C | C | C |
| 19 | B | A | B |
| 20 | B | B | B |
| 21 | B | A | A |
| 22 | B | B | A |
| 23 | B | B | A |
| 24 | B | B | B |
| 25 | B | B | B |
| 26 | B | C | B |
| 27 | B | B | B |
| 28 | B | A | A |
| 29 | B | A | A |
| 30 | B | B | B |
| 31 | B | B | A |
| 32 | B | B | B |
| 33 | B | B | B |
| 34 | C | D | D |
| 35 | C | B | B |
| 36 | B | B | B |
| 37 | B | B | B |
| 38 | B | B | B |
| 39 | B | | B |
| 40 | B | | B |
| 41 | B | | C |
| 42 | C | C | C |
| 43 | B | B | B |
| 44 | B | B | B |
| 45 | B | B | B |
| 46 | C | C | C |
| 47 | B | B | B |
| 48 | B | B | B |
| 49 | B | C | C |
| 50 | B | B | B |
| 51 | C | C | C |
| 52 | B | C | C |
| 53 | B | B | A |
| 54 | B | C | B |
| 55 | B | B | B |
| 56 | B | C | B |
| 57 | B | A | A |
| 58 | | | B |
| 59 | B | B | B |
| 60 | B | B | |
| 61 | B | B | B |
| 62 | B | B | B |
| 63 | B | B | B |
| 64 | | B | B |
| 65 | B | D | C |
| 66 | B | B | B |
| 67 | | B | B |
| 68 | A | C | B |
| 69 | B | B | B |
| 70 | B | B | B |
| 71 | B | B | B |
| 72 | B | B | A |
| 73 | B | B | B |
| 74 | B | B | B |
| 75 | | B | B |
| 76 | B | B | B |
| 77 | B | B | B |
| 78 | | B | B |
| 79 | | B | B |
| 82 | B | B | B |
| 85 | B | C | B |
| 88 | C | B | D |
| 97 | A | A | B |
| 100 | B | B | B |
| 101 | C | B | B |
| 102 | B | B | B |
| 103 | B | B | A |
| 104 | A | B | B |
| 105 | B | A | B |
| 106 | A | B | A |
| 107 | B | A | B |
| 108 | B | B | B |
| 109 | C | B | B |
| 110 | B | A | A |
| 111 | A | B | A |
| 112 | B | | B |
| 113 | B | A | B |
| 114 | B | | B |
| 115 | A | B | B |
| 116 | A | B | B |
| 117 | A | B | B |
| 118 | B | B | B |
| 119 | B | B | B |
| 120 | | A | A |
| 121 | | A | B |
| 122 | | A | B |
| 123 | | B | B |
| 124 | | B | A |
| 125 | | B | A |
| 126 | | A | B |
| 127 | | A | A |
| 128 | | A | A |
| 129 | | A | A |
| 130 | | A | A |
| 131 | | A | A |
| 132 | | A | A |
| 133 | | A | A |
| 134 | | B | B |
| 135 | | A | A |
| 136 | | C | |
| 137 | | A | A |
| 138 | | A | A |
| 139 | | B | A |
| 140 | | A | A |
| 141 | | A | A |
| 142 | | A | A |
| 143 | | A | A |
| 144 | | A | B |
| 145 | | A | B |
| 146 | | B | B |
| 147 | | A | B |
| 148 | | A | A |
| 149 | | B | B |
| 150 | | A | B |
| 151 | | A | B |
| 152 | | A | A |
| 153 | | A | B |
| 154 | | B | B |
| 155 | | A | A |
| 156 | | B | B |
| 157 | | A | A |
| 158 | | B | B |
| 159 | | A | B |
| 160 | | B | B |
| 161 | | A | B |
| 162 | | A | B |
| 163 | | A | A |
| 164 | | B | B |
| 165 | | B | B |
| 166 | | B | C |
| 167 | | B | B |
| 168 | | A | B |

### Solubility in FaSSIF (Fasted State Simulated Intestinal Fluid)

Certain Compounds of the Disclosure were assessed for the apparent solubility in FaSSIF.

0.420 g of NaOH, 3.438 g of NaH₂PO₄ and 6.186 g of NaCl were dissolved into about 900 mL ultrapure water. The solution was adjusted to the pH of 6.5 with 1N NaOH or 1N HCl. The solution was then diluted with ultrapure water to 1000 mL at room temperature. The resulting solution is used as buffer solution.

2.240 g of FaSSIF Powder was added to about 500 mL of above-mentioned buffer solution. The solution was stirred until powder was completely dissolved. Then the solution was diluted with buffer A to 1000 mL at room temperature. This solution is FaSSIF to be used in the following experiment.

15 μL of stock solution (10 mM) of each sample was placed into their proper 96-well rack. 485 μL of FaSSIF was added into each vial of the cap-less solubility sample plate. One stir stick was added to each vial and the vial was sealed using a molded PTFE/Silicone plug. The solubility sample plate was transferred to the Eppendorf Thermomixer Comfort plate shaker and shaked at 25° C. at 1100 RPM for 2 hours. After completion of the 2 hours, the stir sticks using a big magnet were removed. The samples from the solubility sample plate were transferred into the filter plate. Using the vacuum manifold, all the samples are filtered. Aliquot of 5 μL was taken from filtrate followed by addition of 5 μL DMSO and 490 μL of a mixture of H₂O and acetonitrile containing internal standard (1:1).

3 μM Standards solution (STD) was prepared from the 10 mM DMSO STD plate.as following: transferring 6 μL into the remaining empty plate, adding 194 μL of DMSO to that plate to have a STD concentration of 300 μM. From the 300 μM DMSO STD plate, 5 μL solution was transferred into the remaining empty plate. Addition of 5 μL PBS and 490 μL of a mixture of H₂O and acetonitrile containing internal standard (1:1) to that plate furnished a final STD concentration of 3 μM STD solution.

The samples are analyzed and quantified against the standard of known concentration using LC/MS/MS, calculating the solubility of the test compound as following (DF means the dilution factor):

[Sample solubility in FsSSIF (04)]=Area Ratio sample×DF sample×[STD]/Area Ratio STD

The data are listed in Table 3 below. A: Solubility <10 μM; B: 10 μM≤solubility <100 μM; C: Solubility ≥100 μN.

### Liver Microsomal Stability

Certain Compounds of the Disclosure were assessed for ADME metabolic stability in human liver microsomes (HLM).

Samples were prepared by adding 222.5 μL of a master solution (100 mM phosphate buffer and 0.5 mg/mL liver microsomes (HLM)) and 25 μL of a 10 mM NADPH solution to incubation plates, which were then warmed for 10 min. Each compound was separately dissolved in DMSO to prepare 10 mM stock solutions, which were then diluted to 100 μM with acetonitrile. A reaction was started by adding 2.5 μL of the 1001.1M solution of each compound to separate incubation plates such that the final concentration of for each compound in each plate was 1 μM. Aliquots of 30 μL were taken from the reaction solution at 0.5, 5, 15, 30 and 60 minutes. The reaction was stopped by the addition of 5 volumes of cold acetonitrile with IS (100 nM alprazolam, 200 nM caffeine and 100 nM tolbutamide). Aliquot of 100 μL of the supernatant was diluted by 100 μL ultra-pure water, and the mixture was used for LC/MS/MS analysis. Peak areas were determined from extracted ion chromatograms. Peak areas are determined from extracted ion chromatograms. The percentage of remaining parent was calculated from peak area of test compound or PC. The slope value, k, is determined by linear regression of the natural logarithm of percent parent remaining vs. incubation time curve. All calculations are carried out using Microsoft Excel.

The in vitro half-life (in vitro t1/2) is determined from the slope value:

in vitro t_{1/2}=−(0.693/k)

Conversion of the in vitro t1/2 (in min) into the in vitro intrinsic clearance (in vitro CLint, (HLM Clint (μL/min/mg proteins))) is done using the following equation:

$$in vitro {CL}_{int} = \qquad ( \frac{0.693}{( {t}_{1 / 2} )} ) \qquad ⋆ \qquad ( \frac{volume of incubation ( μ L )}{amount of proteins ( mg )} )$$

The data are listed in Table 3 below. A: HLM Clint <10 μL/min/mg; B: 10 μL/min/mg≤HLM Clint <50 μL/min/mg; C: HLM Clint ≥50 μL/min/mg.

TABLE 3
In vitro Data of Certain Exemplary Compounds.

| I# | HLM Clint (μL/min/mg) | Solubility in FaSSIF(μM) |
|---|---|---|
| I-1 | B | |
| I-4 | A | A |
| I-8 | B | C |
| I-11 | B | |
| I-17 | B | |
| I-21 | B | B |
| I-22 | B | C |
| I-23 | B | A |
| I-24 | C | |
| I-25 | C | |
| I-27 | A | A |
| I-31 | B | |
| I-33 | C | |
| I-36 | B | A |
| I-37 | B | |
| I-53 | B | C |
| I-57 | B | |
| I-59 | B | |
| I-64 | A | A |
| I-102 | B | C |
| I-103 | A | A |
| I-106 | A | C |
| I-107 | A | C |
| I-109 | A | C |
| I-97 | B | C |
| I-110 | A | A |
| I-111 | A | C |
| I-113 | B | B |
| I-118 | B | C |
| I-119 | A | C |
| I-120 | A | C |
| I-121 | A | C |
| I-122 | A | B |
| I-123 | B | C |
| I-124 | A | B |
| I-125 | B | C |
| I-126 | B | B |
| I-127 | A | C |
| I-128 | A | B |
| I-129 | B | C |
| I-130 | A | C |
| I-131 | A | C |
| I-132 | B | C |
| I-133 | B | B |
| I-135 | A | C |
| I-137 | B | C |
| I-138 | A | C |
| I-139 | A | C |
| I-140 | A | C |
| I-143 | B | C |
| I-144 | B | A |
| I-145 | A | C |
| I-148 | A | C |
| I-150 | A | B |
| I-151 | A | B |
| I-152 | A | C |
| I-153 | A | C |
| I-155 | A | B |
| I-156 | A | C |
| I-157 | A | C |
| I-163 | A | B |
| I-167 | A | B |
| I-168 | A | B |

### In Vivo Efficacy

Anti-tumor efficacy of USP1 inhibitors were evaluated in mice using the MDA-MB-436 subcutaneous human breast cancer model that harbors the loss-of-function BRCA1 mutation. 6-8 weeks old female NOD SCID mice from Beijing Anikeeper Biotech Co., Ltd. were inoculated subcutaneously with 1×10⁷ MDA-MB-436 tumor cells. When tumors reached a size of approximately 130 mm 3 mice were randomized into group of 5 or 9 and dosed via oral gavage once or twice daily for 28 days with either vehicle (twice daily, 9 animals), one dose of USP1 inhibitor 1-107 at 150 mg/kg (twice daily, 5 animals), three doses of USP1 inhibitor I-111 at 10, or 60 mg/kg (once daily, 9 animals per group), or one dose of USP1 inhibitor 1-124 at 150 mg/kg (twice daily, 5 animals). Body weight and tumor volume were measured twice per week. Tumor volume (TV) was calculated as mean and standard deviation of the mean for each treatment group. The percentage tumor growth inhibition (TGI) was calculated using the formula: TGI=(RTV_{vehicle}−RTVₜᵣₑₐₜₘₑₙₜ)*100)/(RTV_{vehicle} −1), where RTV is the mean relative volumes (RTV=TV_{Day terminal}-TV_{Day 0}) of the tumors in the treatment and the vehicle groups, respectively, on a given day from Day 0-Day 28 where Day 0 is the first day of treatment.

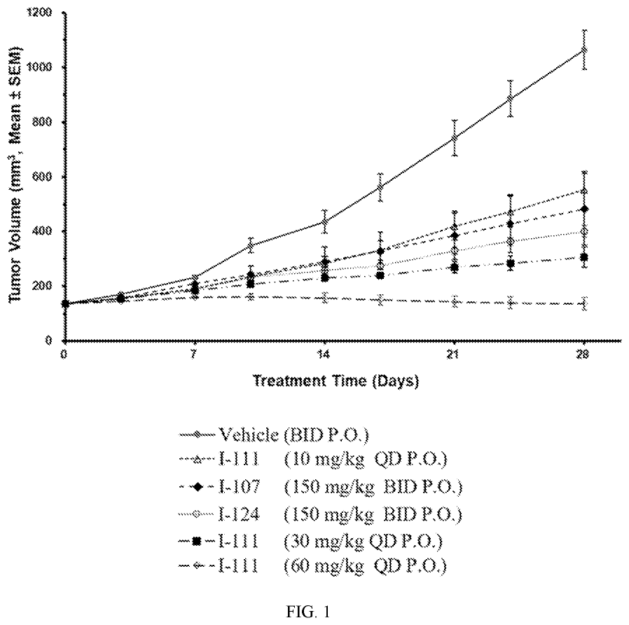

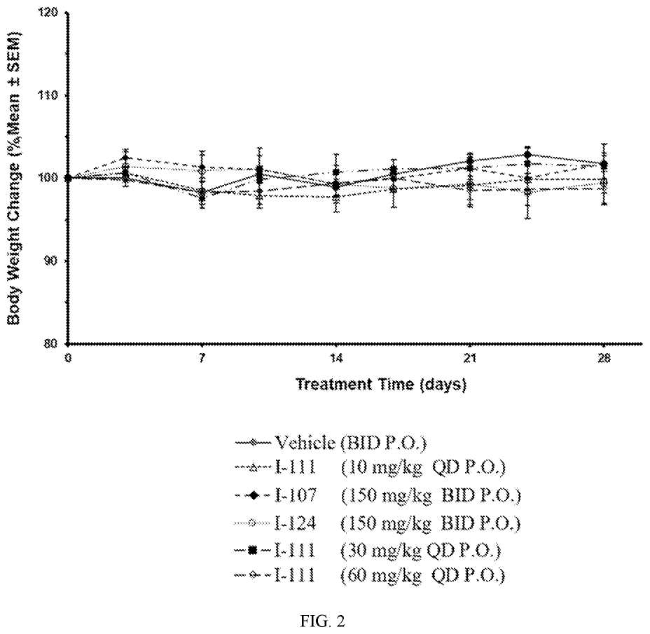

As shown in FIG. 1, slightly >100% TGI was achieved with higher QD dose of I-111 (60 mg/kg), and there were proportionally reduced TGIs of ˜55% and ˜80% with lower QD doses of I-111 at 10 and 30 mg/kg, respectively. TGI of ˜70% and ˜60% was observed with BID dose at 150 mg/kg for 1-124 and 1-107, respectively. All treatment groups were well-tolerated, with various dose levels and dosing frequency for the duration of the treatment period from day 0 to day 28 in the tumor bearing mice, as shown in FIG. 2.

While we have described a number of embodiments of this invention, it is apparent that our basic examples may be altered to provide other embodiments that utilize the compounds and methods of this invention. Therefore, it will be appreciated that the scope of this invention is to be defined by the application and appended claims rather than by the specific embodiments that have been represented by way of example.

1. A compound of formula I: 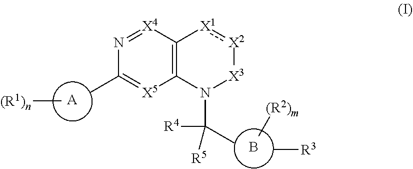
or a pharmaceutically acceptable salt thereof, wherein: [symbol]
is a single bond or a double bond;
X¹ is O, N, N(R¹¹), C(O), CR¹², or C(R¹²)₂;
X² is O, N, N(R¹¹), C(O), CR¹², or C(R¹²)₂;
X³ is N(R¹¹), C(O), or C(R¹²)₂;
X⁴ is N or CR¹²;
X⁵ is N or CR¹²;
each R¹¹ is independently hydrogen, deuterium, R, or —C(O)—R;
each R¹² is independently hydrogen, deuterium, halogen, R, —OR, —NHR, —N(R)₂, —C(O)—R, —COOR, —C(O)—NHR, or —C(O)—N(R)₂;
Ring A is a 5-6 membered heteroaromatic ring having 1-4 heteroatoms selected from N, O, and S;
each R¹ is independently halogen, R, —OR, —NHR, —N(R)₂, —C(O)—R, —C(O)—NHR, or —C(O)—N(R)₂;
Ring B is selected from a phenyl ring, a 5-6 membered heteroaromatic ring having 1-4 heteroatoms selected from N, O, and S, or a 5-6 membered heterocyclyl ring having 1-4 heteroatoms selected from N, O, and S;
each R² is independently halogen, R, —OR, —NHR, —N(R)₂, or —C(O)—R;
R³ is optionally substituted phenyl, or optionally substituted 5-6 membered heteroaryl having 1-4 heteroatoms selected from N, O, and S;
each of R⁴ and R⁵ is independently hydrogen, deuterium, halogen, R, —OR, —NHR, or —N(R)₂; or
R⁴ and R⁵, together with the atom to which they attach, form an optionally substituted ring selected from a C₃-C₇ carbocyclic ring or a 3-7 membered heterocyclic ring having 1-4 heteroatoms selected from N, O, and S;
each R is independently optionally substituted C₁₋₆ aliphatic, or an optionally substituted ring selected from a phenyl ring, a 5-6 membered heteroaromatic ring having 1-4 heteroatoms selected from N, O, and S, a C₃-C₇ carbocyclic ring, or a 3-7 membered heterocyclic ring having 1-4 heteroatoms selected from N, O, or S;
n is 0, 1, 2 or 3; or
m is 0, 1, 2 or 3.

2. The compound of claim 1, wherein the compound is of formula II or II′: 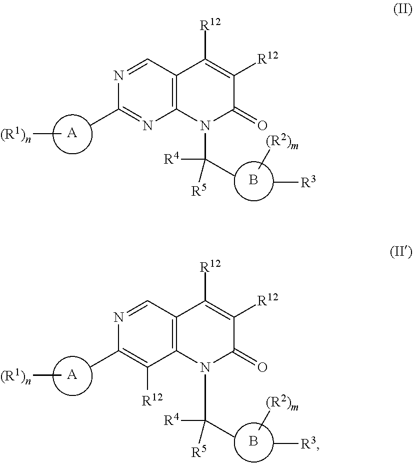
optionally wherein the compound of formula II is a compound of formulae (II-1), (II-2), (II-3), 01-4), (II-6), (II-7), (II-8), or (II-9):

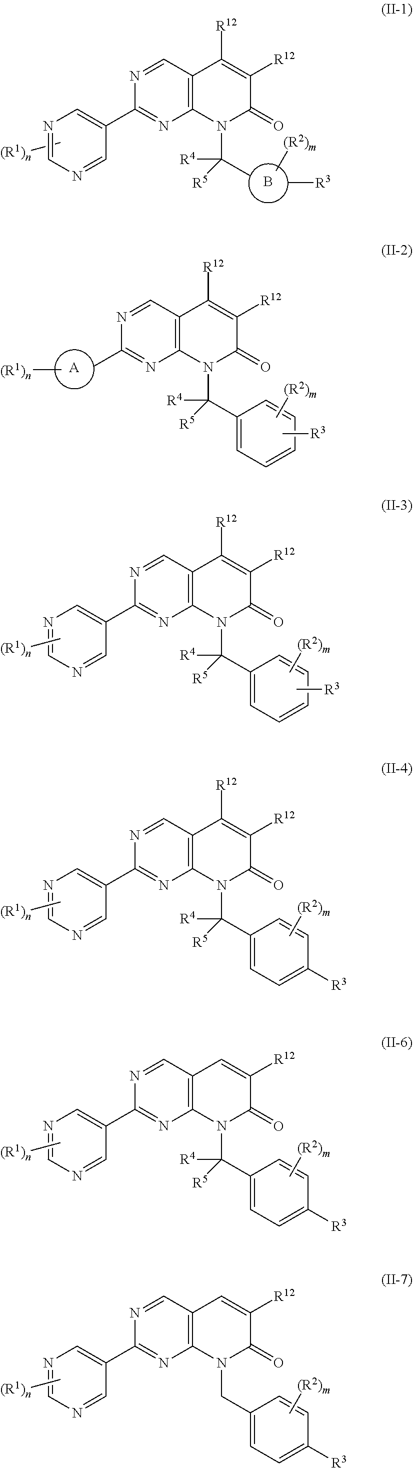

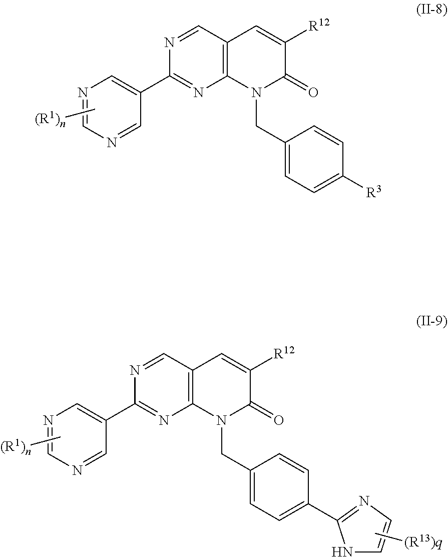

3. The compound of claim 1, wherein the compound is of formula III or III′:

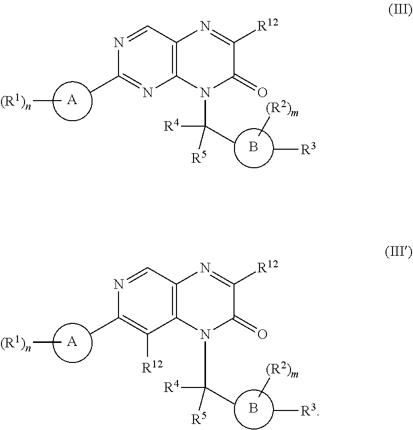

4. The compound of claim 1, wherein the compound is of formula IV or IV′: 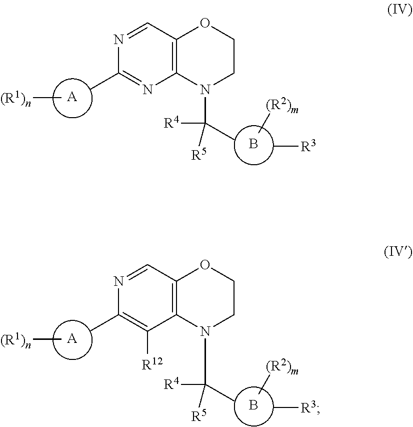
or wherein the compound is of formula V or V′: 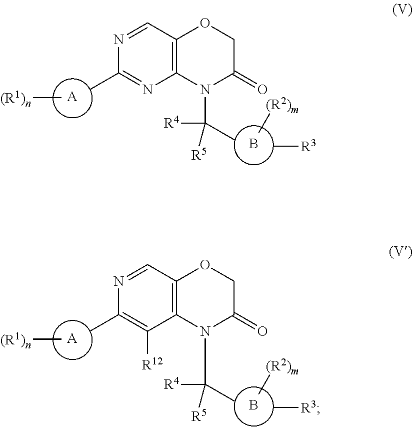
or wherein the compound is of formula VI or VI′: 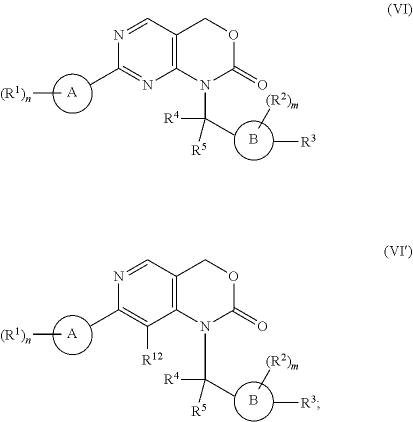
or wherein the compound is of formula VII or VII′: 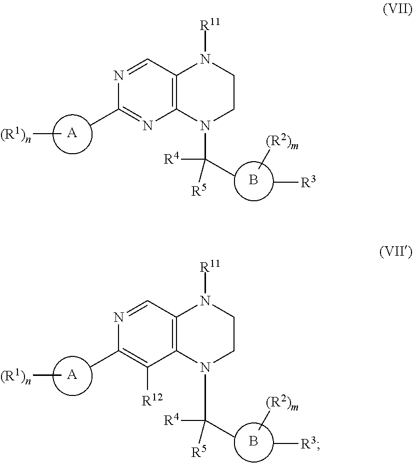
or wherein the compound is of formula VIII or VIII′: 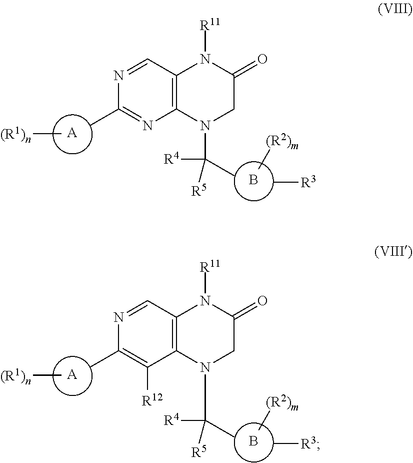
or wherein the compound is of formula IX or IX′: 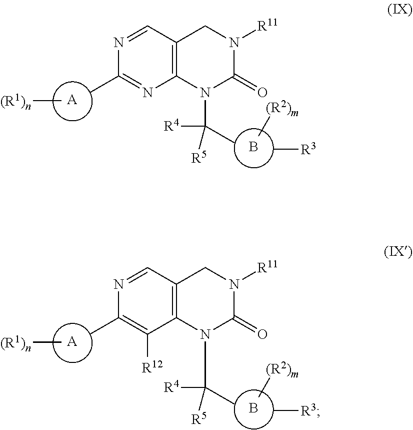
or wherein the compound is of formula X or X′: 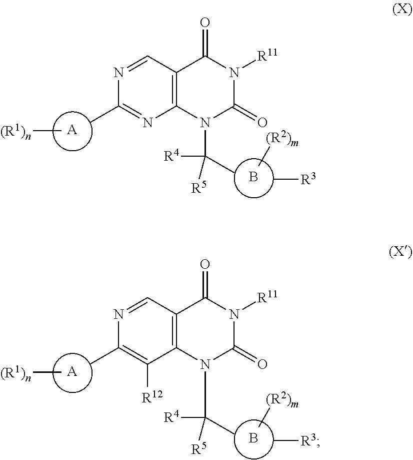
or wherein the compound is of formula XI or XI′:

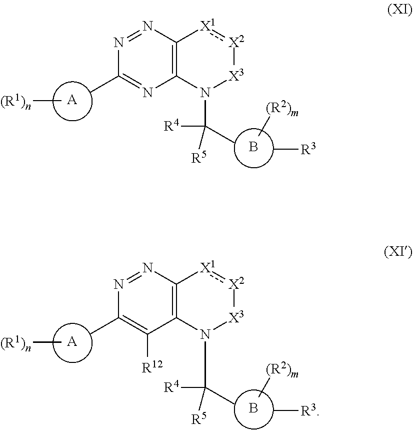

5-11. (canceled)

12. The compound of claim 1, wherein the compound is of formula XII or XII′: 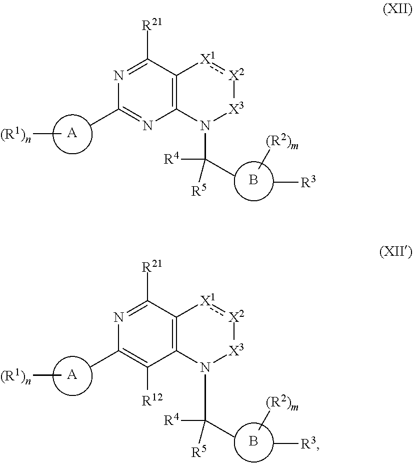
wherein R²¹ is optionally substituted C₁₋₆ aliphatic.

13. The compound of claim 1, wherein the compound is of formula XIII or XIII′: 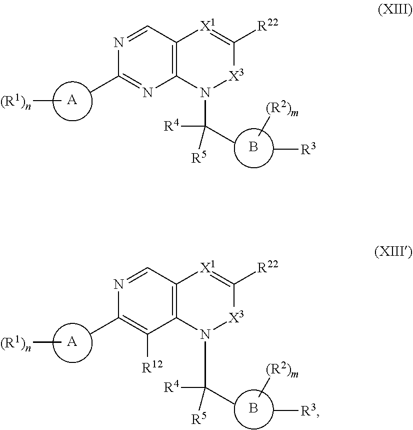
wherein R²² is an optionally substituted ring selected from a phenyl ring and a 5-6 membered heteroaromatic ring having 1-4 heteroatoms selected from N, O, and S.

14. The compound of claim 1, wherein the compound is of formula XIV or XIV′: 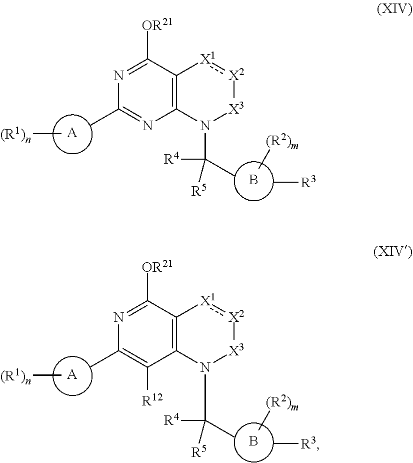
wherein R²¹ is optionally substituted C₁₋₆ aliphatic.

15. The compound of claim 1, wherein each R¹¹ is independently hydrogen or optionally substituted C₁₋₆ aliphatic.

16. The compound of claim 1, wherein each R¹² is independently hydrogen, halogen, optionally substituted C₁₋₆ aliphatic, —C(O)—R, —COOR, or —C(O)—N(R)₂.

17. The compound of claim 1, wherein Ring A is a 5-6 membered heteroaromatic ring having 1-4 heteroatom N.

18. The compound of claim 17, wherein Ring A is

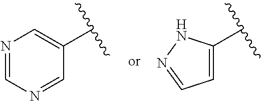 [SMILES: *c1cncnc1 ; *c1ccn[nH]1]

19. The compound of claim 1, wherein each R¹ is independently halogen, R, or —OR.

20. The compound of claim 19, wherein each R¹ is independently —Cl, —F, —OCH₃, —OCD₃ 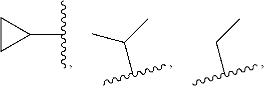
—O—CHF₂, —O—CF₃, or

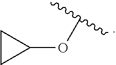 [SMILES: *OC1CC1]

21. The compound of claim 1, wherein Ring B is a phenyl ring, a 6 membered heteroaromatic ring having 1-4 heteroatom N, or a 6 membered heterocyclyl ring having 1-4 heteroatom N.

22. The compound of claim 21, wherein Ring B is

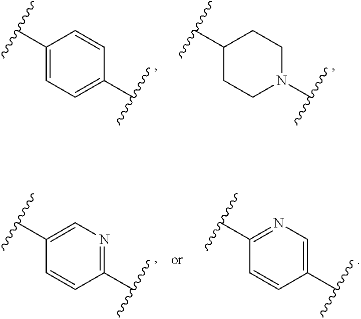 [SMILES: *c1ccc(*)cc1 ; *C1CCN(*)CC1 ; *c1ccc(*)nc1 ; *c1ccc(*)nc1]

23. The compound of claim 1, wherein each R² is independently halogen, R, —OR, or —C(O)—R.

24. The compound of claim 23, wherein each R² is independently —F, —CH₃, —CD₃, —OCH₃, or —OCD₃.

25. The compound of claim 1, wherein R³ is optionally substituted

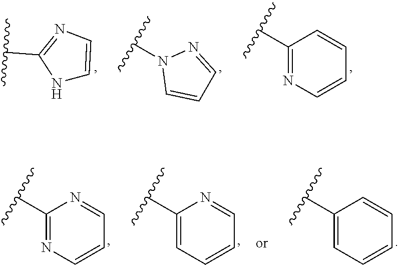

26. The compound of claim 25, wherein R³ is 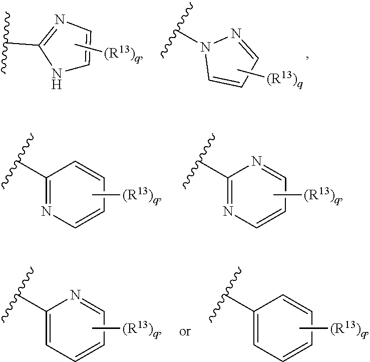
wherein each R¹³ is independently halogen, R, or —OR, and q is 0, 1, 2, or 3.

27. The compound of claim 26, wherein R¹³ is —F, —CH₃, —CD₃, —CHF₂, —CF₃, —OCH₃, —OCD₃,

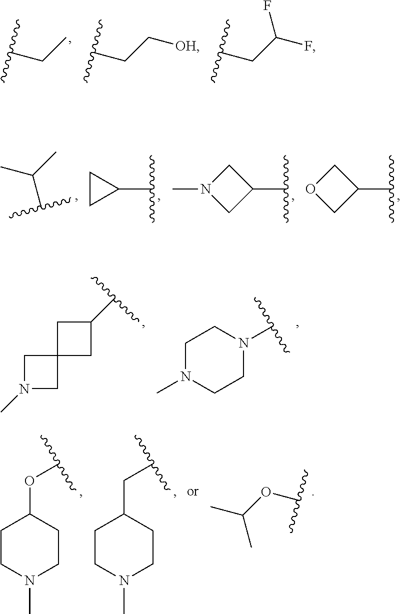

28. The compound of claim 26, wherein R³ is

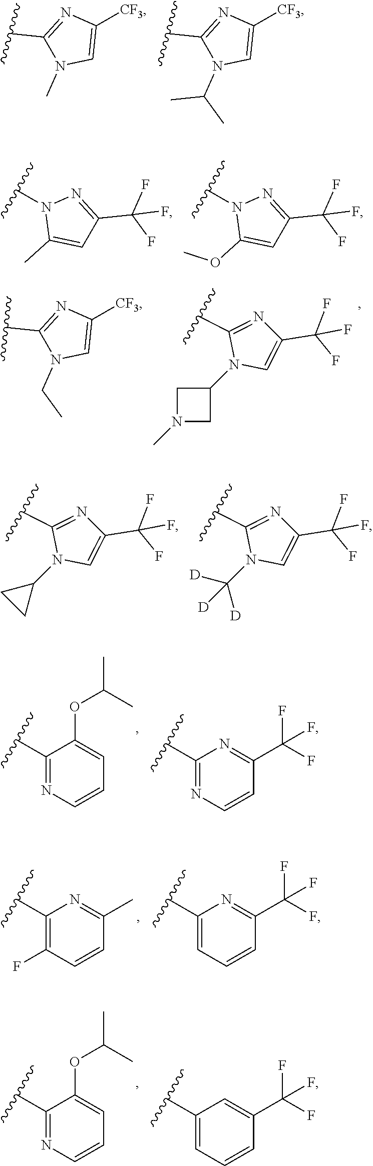

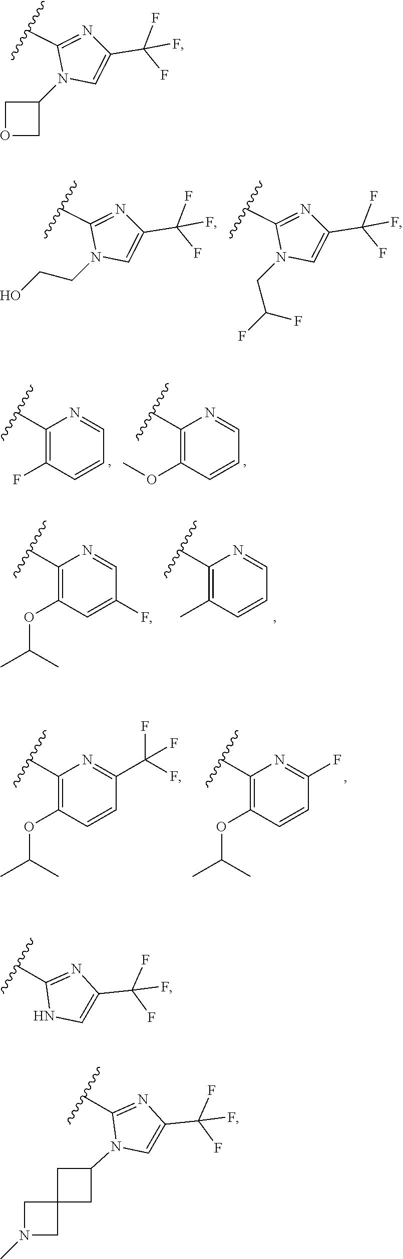

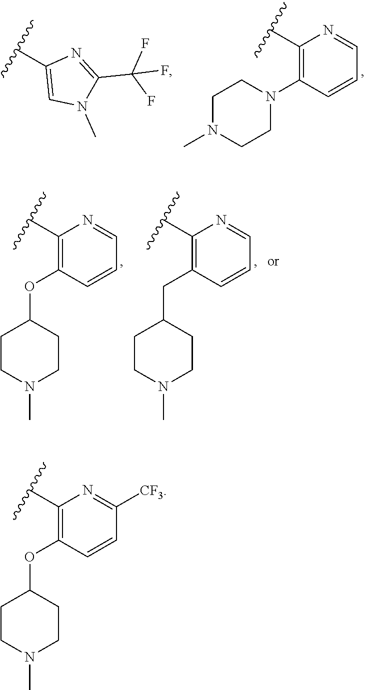 [SMILES: *c1cn(C)c(C(F)(F)F)n1 ; *c1ncccc1N1CCN(C)CC1 ; *c1ncccc1OC1CCN(C)CC1 ; *c1ncccc1CC1CCN(C)CC1 ; *c1nc(C(F)(F)F)ccc1OC1CCN(C)CC1]

29. The compound of claim 1, wherein each of R⁴ and R⁵ is independently hydrogen, deuterium, or optionally substituted C₁₋₆ aliphatic.

30. A compound selected from those as set forth in Table 1, or a pharmaceutically acceptable salt thereof.

31. A pharmaceutical composition comprising the compound of claim 1, or a pharmaceutically acceptable salt thereof, and a pharmaceutically acceptable carrier, adjuvant, or vehicle.

32. A method for treating a disease or disorder associated with ubiquitin-specific-processing Protease 1 (USP1) in a patient, the method comprising administering to the patient the compound of claim 1, or a pharmaceutically acceptable salt thereof.

33. (canceled)
